SEQUENCE LISTING (1168 sequences)

SEQ ID NO: 1 - protein, 133 aa, Homo sapiens [sequence omitted]

SEQ ID NO: 2 - protein, 133 aa, Homo sapiens
  REGION 1..133: note = misc_feature - h IL-2 (C125S), cytokine
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 3 - protein, 149 aa, Mus musculus
  REGION 1..149: note = misc_feature - m IL-2, cytokine
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 4 - protein, 149 aa, Mus musculus
  REGION 1..149: note = misc_feature - m IL-2 (C140S), cytokine
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFSQSIISTSPQ

SEQ ID NO: 5-9 - skipped (5 entries)

SEQ ID NO: 10 - protein, 219 aa, Homo sapiens
  REGION 1..219: note = misc_feature - h IL-2Ralpha
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 11 - protein, 63 aa, Homo sapiens
  REGION 1..63: note = misc_feature - h IL-2Ralpha (1-63)
ELCDDDPPEIPHATFKAMAYKEGTMLNCECKRGFRRIKSGSLYMLCTGNSSHSSWDNQCQCTS

SEQ ID NO: 12 - protein, 219 aa, Homo sapiens
  REGION 1..219: note = misc_feature - h IL-2Ralpha (M25I)
ELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 13 - protein, 219 aa, Homo sapiens
  REGION 1..219: note = misc_feature - h IL-2Ralpha (L42v)
ELCDDDPPEIPHATFKAMAYKEGTMLNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 14 - protein, 219 aa, Homo sapiens
  REGION 1..219: note = misc_feature - h IL-2Ralpha (M25I; L42v)
ELCDDDPPEIPHATFKAMAYKEGTILNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 15 - protein, 240 aa, Homo sapiens
  REGION 1..240: note = misc_feature - Human IL2Rgamma polypeptide sequence
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 16 - protein, 214 aa, Homo sapiens
  REGION 1..214: note = misc_feature - Human IL2Rbeta polypeptide sequence
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 17 - protein, 214 aa, synthetic construct
  REGION 1..214: note = Synthetic: chimeric IL-2Ralpha
ELCLYDPPEVPNATFKALSYKNGTILNCECKRGFRRLKELVYMRCLGNSWSSNCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 18 - protein, 215 aa, Mus musculus
  REGION 1..215: note = misc_feature - m IL-2Ralpha
ELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 19 - protein, 58 aa, Mus musculus
  REGION 1..58: note = misc_feature - m IL-2Ralpha (1-58)
ELCLYDPPEVPNATFKALSYKNGTILNCECKRGFRRLKELVYMRCLGNSWSSNCQCTS

SEQ ID NO: 20 - protein, 219 aa, Homo sapiens
  REGION 1..219: note = misc_feature - h IL-2Ralpha (1-219) M25I/D4L/D5Y
ELCLYDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 21 - protein, 219 aa, Homo sapiens
  REGION 1..219: note = misc_feature - h IL-2Ralpha (1-219) L42v/D4L/D5Y
ELCLYDPPEIPHATFKAMAYKEGTMLNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 22 - protein, 219 aa, Homo sapiens
  REGION 1..219: note = misc_feature - h IL-2Ralpha (1-219) M25I/L42v/D4L/D5Y
ELCLYDPPEIPHATFKAMAYKEGTILNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 23 - protein, 219 aa, Homo sapiens
  REGION 1..219: note = misc_feature - h IL-2Ralpha (1-219)D4L/D5Y
ELCLYDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 24 - protein, 218 aa, Homo sapiens
  REGION 1..218: note = misc_feature - h IL-2Ralpha (1-219) SGSL39-42ELv
ELCDDDPPEIPHATFKAMAYKEGTMLNCECKRGFRRIKELVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 25 - protein, 192 aa, Homo sapiens
  REGION 1..192: note = misc_feature - h IL-2Ralpha (1-192)
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 26 - protein, 192 aa, Homo sapiens
  REGION 1..192: note = misc_feature - h IL-2Ralpha (1-192)M25I
ELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 27 - protein, 192 aa, Homo sapiens
  REGION 1..192: note = misc_feature - h IL-2Ralpha (1-192)L42v
ELCDDDPPEIPHATFKAMAYKEGTMLNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 28 - protein, 192 aa, Homo sapiens
  REGION 1..192: note = misc_feature - h IL-2Ralpha (1-192)D4L/D5Y
ELCLYDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 29 - protein, 191 aa, Homo sapiens
  REGION 1..191: note = misc_feature - h IL-2Ralpha (1-192) SGSL39-42ELv
ELCDDDPPEIPHATFKAMAYKEGTMLNCECKRGFRRIKELVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 30 - protein, 247 aa, Homo sapiens
  REGION 1..247: note = misc_feature - scFv2
QSVLTQPPSVSGAPGQRVTISCTGTSSNIGAHYDVHWYQQFPGTAPKRLIYGNNNRPSGVPARFSGSKSGTSASLAITGLQAEDEADYYCQSYDRSLRGWVFGGGTKLTVLGEGKSSGSGSESKASEVQLVESGGGLVQPGRSLRLSCAASGFTFDDYAMHWVRQAPGKGLEWVSGISWNSGSIGYADSVKGRFTISRDNSKNTLYLQMNSLRAEDTAVYYCAKDVNWNYGYYFDYWGQGTLVTVSS

SEQ ID NO: 31 - protein, 251 aa, Homo sapiens
  REGION 1..251: note = misc_feature - scFv2 (18mer linker)
QSVLTQPPSVSGAPGQRVTISCTGTSSNIGAHYDVHWYQQFPGTAPKRLIYGNNNRPSGVPARFSGSKSGTSASLAITGLQAEDEADYYCQSYDRSLRGWVFGGGTKLTVLGGSTSGSGKPGSGEGSTKGEVQLVESGGGLVQPGRSLRLSCAASGFTFDDYAMHWVRQAPGKGLEWVSGISWNSGSIGYADSVKGRFTISRDNSKNTLYLQMNSLRAEDTAVYYCAKDVNWNYGYYFDYWGQGTLVTVSS

SEQ ID NO: 32 - protein, 112 aa, Homo sapiens
  REGION 1..112: note = misc_feature - vL region of scFv2
QSVLTQPPSVSGAPGQRVTISCTGTSSNIGAHYDVHWYQQFPGTAPKRLIYGNNNRPSGVPARFSGSKSGTSASLAITGLQAEDEADYYCQSYDRSLRGWVFGGGTKLTVLG

SEQ ID NO: 33 - protein, 121 aa, Homo sapiens
  REGION 1..121: note = misc_feature - vH region of scFv2
EVQLVESGGGLVQPGRSLRLSCAASGFTFDDYAMHWVRQAPGKGLEWVSGISWNSGSIGYADSVKGRFTISRDNSKNTLYLQMNSLRAEDTAVYYCAKDVNWNYGYYFDYWGQGTLVTVSS

SEQ ID NO: 34 - protein, 14 aa, synthetic construct
  REGION 1..14: note = Synthetic: scFv2 vL HvR1
TGTSSNIGAHYDVH

SEQ ID NO: 35 - protein, 7 aa, synthetic construct
  REGION 1..7: note = Synthetic: scFv2 vL HvR2
GNNNRPS

SEQ ID NO: 36 - protein, 11 aa, synthetic construct
  REGION 1..11: note = Synthetic: scFv2 vL HvR3
QSYDRSLRGWV

SEQ ID NO: 37 - protein, 6 aa, synthetic construct
  REGION 1..6: note = Synthetic: scFv2 vH HvR1
DDYAMH

SEQ ID NO: 38 - protein, 17 aa, synthetic construct
  REGION 1..17: note = Synthetic: scFv2 vH HvR2
GISWNSGSIGYADSVKG

SEQ ID NO: 39 - protein, 13 aa, synthetic construct
  REGION 1..13: note = Synthetic: scFv2 vH HvR3
KDVNWNYGYYFDY

SEQ ID NO: 40 - protein, 192 aa, Homo sapiens
  REGION 1..192: note = misc_feature - h IL-2Ralpha (1-192)M25I/D4L/D5Y
ELCLYDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 41 - protein, 192 aa, Homo sapiens
  REGION 1..192: note = misc_feature - h IL-2Ralpha (1-192)M25I/L42v
ELCDDDPPEIPHATFKAMAYKEGTILNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 42 - protein, 192 aa, Homo sapiens
  REGION 1..192: note = misc_feature - h IL-2Ralpha (1-192) D4L/D5Y/L42v
ELCLYDPPEIPHATFKAMAYKEGTMLNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 43 - protein, 192 aa, Homo sapiens
  REGION 1..192: note = misc_feature - h IL-2Ralpha (1-192) M25I/D4L/D5Y/L42v
ELCLYDPPEIPHATFKAMAYKEGTILNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 44 - protein, 178 aa, Homo sapiens
  REGION 1..178: note = misc_feature - h IL-2Ralpha (1-178)
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKP

SEQ ID NO: 45 - protein, 178 aa, Homo sapiens
  REGION 1..178: note = misc_feature - h IL-2Ralpha (1-178) M25I
ELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKP

SEQ ID NO: 46 - protein, 178 aa, Homo sapiens
  REGION 1..178: note = misc_feature - h IL-2Ralpha (1-178) L42v
ELCDDDPPEIPHATFKAMAYKEGTMLNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKP

SEQ ID NO: 47 - protein, 178 aa, Homo sapiens
  REGION 1..178: note = misc_feature - h IL-2Ralpha (1-178) D4L/D5Y
ELCLYDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKP

SEQ ID NO: 48 - protein, 177 aa, Homo sapiens
  REGION 1..177: note = misc_feature - h IL-2Ralpha (1-178) SGSL39-42ELv
ELCDDDPPEIPHATFKAMAYKEGTMLNCECKRGFRRIKELVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKP

SEQ ID NO: 49 - protein, 178 aa, Homo sapiens
  REGION 1..178: note = misc_feature - h IL-2Ralpha (1-178) M25I/L42v
ELCDDDPPEIPHATFKAMAYKEGTILNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKP

SEQ ID NO: 50 - protein, 178 aa, Homo sapiens
  REGION 1..178: note = misc_feature - h IL-2Ralpha (1-178) D4L/D5Y/L42v
ELCLYDPPEIPHATFKAMAYKEGTMLNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKP

SEQ ID NO: 51 - protein, 178 aa, Homo sapiens
  REGION 1..178: note = misc_feature - h IL-2Ralpha (1-178) D4L/D5Y/M25I/ L42v
ELCLYDPPEIPHATFKAMAYKEGTILNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKP

SEQ ID NO: 52-69 - skipped (18 entries)

SEQ ID NO: 70 - protein, 226 aa, Homo sapiens
  REGION 1..226: note = misc_feature - h IgG1 Fc,C-terminal K residue deleted
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 226 aa, Homo sapiens
  REGION 1..226: note = misc_feature - Human IgG1 K392D K409D Fc domain polypeptide sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 72 - protein, 591 aa, Homo sapiens
  REGION 1..591: note = misc_feature - Human serum albumin
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 73 - protein, 222 aa, Mus musculus
  REGION 1..222: note = misc_feature - m IgG1 Fc
GCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 74 - protein, 221 aa, Mus musculus
  REGION 1..221: note = misc_feature - Murine IgG1 T252M Fc domain polypeptide sequence
GCKPCICTVPEVSSVFIFPPKPKDVLMITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 75 - protein, 231 aa, Homo sapiens
  REGION 1..231: note = misc_feature - hIgG1 Fc knob L234A/L235A
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 231 aa, Homo sapiens
  REGION 1..231: note = misc_feature - hIgG1 Fc knob (L234A/L235A, H435R, Y436F)
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 77 - protein, 231 aa, Homo sapiens
  REGION 1..231: note = misc_feature - hIgG1 Fc hole L234A/L235A
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 78 - protein, 231 aa, Homo sapiens
  REGION 1..231: note = misc_feature - hIgG1 Fc hole (L234A/L235A, H435R, Y436F)
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 79 - skipped

SEQ ID NO: 80 - protein, 7 aa, synthetic construct
  REGION 1..7: note = Synthetic: MMP cleavage site polypeptide sequence
GPLGVRG

SEQ ID NO: 81 - protein, 7 aa, synthetic construct
  REGION 1..7: note = Synthetic: G112631 polypeptide sequence
GPLGVRG

SEQ ID NO: 82 - protein, 7 aa, synthetic construct
  REGION 1..7: note = Synthetic: G112632polypeptide sequence
GPLGLRG

SEQ ID NO: 83 - protein, 7 aa, synthetic construct
  REGION 1..7: note = Synthetic: G112633polypeptide sequence
GPLGLAR

SEQ ID NO: 84 - protein, 8 aa, synthetic construct
  REGION 1..8: note = Synthetic: G112634polypeptide sequence
GPAALVGA

SEQ ID NO: 85 - protein, 8 aa, synthetic construct
  REGION 1..8: note = Synthetic: G112635polypeptide sequence
GPAALIGG

SEQ ID NO: 86 - protein, 8 aa, synthetic construct
  REGION 1..8: note = Synthetic: G112636polypeptide sequence
GPLNLVGR

SEQ ID NO: 87 - protein, 8 aa, synthetic construct
  REGION 1..8: note = Synthetic: G112637polypeptide sequence
GPAGLVAD

SEQ ID NO: 88 - protein, 8 aa, synthetic construct
  REGION 1..8: note = Synthetic: G112638polypeptide sequence
GPANLVAP

SEQ ID NO: 89 - protein, 8 aa, synthetic construct
  REGION 1..8: note = Synthetic: G112639polypeptide sequence
VPLSLYSG

SEQ ID NO: 90 - protein, 10 aa, synthetic construct
  REGION 1..10: note = Synthetic: G112640polypeptide sequence
SGESPAYYTA

SEQ ID NO: 91-119 - skipped (29 entries)

SEQ ID NO: 120 - protein, 10 aa, synthetic construct
  REGION 1..10: note = Synthetic: Gly-Ser rich linker polypeptide sequence
SGGGGSGGGG

SEQ ID NO: 121-178 - skipped (58 entries)

SEQ ID NO: 179 - protein, 7 aa, synthetic construct
  REGION 1..7: note = Synthetic: denatured collagen Iv; binding to MMP degraded collagen
TLTYTWS

SEQ ID NO: 180 - protein, 5 aa, synthetic construct
  REGION 1..5: note = Synthetic: denatured collagen Iv; binding to MMP degraded collagen
CREKA

SEQ ID NO: 181 - skipped

SEQ ID NO: 182 - protein, 40 aa, synthetic construct
  REGION 1..40: note = Synthetic: denatured Collagen ; from Fibronectin Domain 1-6
GHCVTDSGVVYSVGMQWLKTQGNKQMLCTCLGNGVSCQET

SEQ ID NO: 183 - protein, 44 aa, synthetic construct
  REGION 1..44: note = Synthetic: denatured Collagen ; from Fibronectin Domain 1-7
EICTTNEGVMYRIGDQWDKQHDMGHMMRCTCVGNGRGEWTCIAY

SEQ ID NO: 184 - protein, 43 aa, synthetic construct
  REGION 1..43: note = Synthetic: denatured Collagen ; from Fibronectin Domain 1-8
DQCIVDDITYNVNDTFHKRHEEGHMLNCTCFGQGRGRWKCDPV

SEQ ID NO: 185 - protein, 44 aa, synthetic construct
  REGION 1..44: note = Synthetic: denatured Collagen ; from Fibronectin Domain 1-9
DQCQDSETGTFYQIGDSWEKYVHGVRYQCYCYGRGIGEWHCQPL

SEQ ID NO: 186 - protein, 49 aa, synthetic construct
  REGION 1..49: note = Synthetic: denatured Collagen ; from Fibronectin Domain 2-1
SNGEPCVLPFTYNGRTFYSCTTEGRQDGHLWCSTTSNYEQDQKYSFCTD

SEQ ID NO: 187 - protein, 49 aa, synthetic construct
  REGION 1..49: note = Synthetic: denatured Collagen ; from Fibronectin Domain 2-2
SNGALCHFPFLYNNHNYTDCTSEGRRDNMKWCGTTQNYDADQKFGFCPM

SEQ ID NO: 188 - protein, 9 aa, synthetic construct
  REGION 1..9: note = Synthetic: Collagen type I
LRELHLDNN

SEQ ID NO: 189 - protein, 20 aa, synthetic construct
  REGION 1..20: note = Synthetic: Collagen type I
RRANAALKAGELYKSILYGC

SEQ ID NO: 190 - protein, 20 aa, synthetic construct
  REGION 1..20: note = Synthetic: Collagen type I
RRANAALKAGELYKCILYGC

SEQ ID NO: 191 - protein, 26 aa, synthetic construct
  REGION 1..26: note = Synthetic: Collagen type I
MIVIELGTNPLKSSGIENGAFQGMKK

SEQ ID NO: 192 - protein, 9 aa, synthetic construct
  REGION 1..9: note = Synthetic: Collagen type I
LRELHLNNN

SEQ ID NO: 193 - protein, 10 aa, synthetic construct
  REGION 1..10: note = Synthetic: Collagen type I
WREPSFCALS

SEQ ID NO: 194 - protein, 7 aa, synthetic construct
  REGION 1..7: note = Synthetic: Collagen type I
TKKTLRT

SEQ ID NO: 195 - protein, 13 aa, synthetic construct
  REGION 1..13: note = Synthetic: Collagen type I
CPKESCNLFVLKD

SEQ ID NO: 196 - protein, 10 aa, synthetic construct
  REGION 1..10: note = Synthetic: Collagen type I
WREPSFCALS

SEQ ID NO: 197 - protein, 11 aa, synthetic construct
  REGION 1..11: note = Synthetic: Collagen type I, H-v-F/W-Q/ M-Q-P/A-P/K motif
HVWMQAPGGGK

SEQ ID NO: 198 - protein, 11 aa, synthetic construct
  REGION 1..11: note = Synthetic: Collagen type I
HVWMQAPGGGC

SEQ ID NO: 199 - protein, 6 aa, synthetic construct
  REGION 1..6: note = Synthetic: Collagen type II
WYRGRL

SEQ ID NO: 200 - protein, 7 aa, synthetic construct
  REGION 1..7: note = Synthetic: Collagen type Iv
KLWVLPK

SEQ ID NO: 201 - protein, 18 aa, synthetic construct
  REGION 1..18: note = Synthetic: Collagen
RRANAALKAGELYKSILY

SEQ ID NO: 202 - protein, 9 aa, synthetic construct
  REGION 1..9: note = Synthetic: Collagen
GELYKSILY

SEQ ID NO: 203 - protein, 18 aa, synthetic construct
  REGION 1..18: note = Synthetic: Collagen
RRANAALKAGELYKCILY

SEQ ID NO: 204 - protein, 9 aa, synthetic construct
  REGION 1..9: note = Synthetic: Collagen
GELYKCILY

SEQ ID NO: 205 - protein, 9 aa, synthetic construct
  REGION 1..9: note = Synthetic: Collagen
RLDGNEIKR

SEQ ID NO: 206 - protein, 13 aa, synthetic construct
  REGION 1..13: note = Synthetic: Collagen
AHEEISTTNEGVM

SEQ ID NO: 207 - protein, 28 aa, synthetic construct
  REGION 1..28: note = Synthetic: Collagen
NGVFKYRPRYFLYKHAYFYPPLKRFPVQ

SEQ ID NO: 208 - protein, 10 aa, synthetic construct
  REGION 1..10: note = Synthetic: Collagen
CQDSETRTFY

SEQ ID NO: 209 - protein, 7 aa, synthetic construct
  REGION 1..7: note = Synthetic: Collagen
TKKTLRT

SEQ ID NO: 210 - protein, 28 aa, synthetic construct
  REGION 1..28: note = Synthetic: Collagen
GLRSKSKKFRRPDIQYPDATDEDITSHM

SEQ ID NO: 211 - protein, 7 aa, synthetic construct
  REGION 1..7: note = Synthetic: Collagen
SQNPVQP

SEQ ID NO: 212 - protein, 11 aa, synthetic construct
  REGION 1..11: note = Synthetic: Collagen
SYIRIADTNIT

SEQ ID NO: 213 - protein, 8 aa, synthetic construct
  REGION 1..8: note = Synthetic: Collagen
KELNLVYT

SEQ ID NO: 214 - protein, 4 aa, synthetic construct
  REGION 1..4: note = Synthetic: Collagen
GSIT

SEQ ID NO: 215 - protein, 12 aa, synthetic construct
  REGION 1..12: note = Synthetic: Collagen
GSITTIDVPWNV

SEQ ID NO: 216 - protein, 9 aa, synthetic construct
  REGION 1..9: note = Synthetic: Collagen
GQLYKSILY

SEQ ID NO: 217 - protein, 18 aa, synthetic construct
  REGION 1..18: note = Synthetic: Collagen
RRANAALKAGQLYKSILY

SEQ ID NO: 218 - protein, 10 aa, synthetic construct
  REGION 1..10: note = Synthetic: Collagen
WREPSFCALS

SEQ ID NO: 219 - protein, 14 aa, synthetic construct
  REGION 1..14: note = Synthetic: Collagen
WHCTTKFPHHYCLY

SEQ ID NO: 220 - protein, 16 aa, synthetic construct
  REGION 1..16: note = Synthetic: Collagen
AHKCPWHLYTTHYCFT

SEQ ID NO: 221 - protein, 16 aa, synthetic construct
  REGION 1..16: note = Synthetic: Collagen
PAHKCPWHLYTHYCFT

SEQ ID NO: 222 - protein, 6 aa, synthetic construct
  REGION 1..6: note = Synthetic: Collagen
  SITE 3: note = misc_feature - X is 4-hydroxyproline
GRXGER

SEQ ID NO: 223 - protein, 6 aa, synthetic construct
  REGION 1..6: note = Synthetic: Collagen
  SITE 3: note = misc_feature - X is 4-hydroxyproline
GMXGER

SEQ ID NO: 224 - protein, 6 aa, synthetic construct
  REGION 1..6: note = Synthetic: Collagen
  SITE 3: note = misc_feature - 4-hydroxyproline
GLXGEN

SEQ ID NO: 225 - protein, 6 aa, synthetic construct
  REGION 1..6: note = Synthetic: Collagen
  SITE 3: note = misc_feature - X is 4-hydroxyproline
GLXGER

SEQ ID NO: 226 - protein, 6 aa, synthetic construct
  REGION 1..6: note = Synthetic: Collagen
GLKGEN

SEQ ID NO: 227 - protein, 15 aa, synthetic construct
  REGION 1..15: note = Synthetic: Collagen
  SITE 3: note = misc_feature - X is 4-hydroxyproline
  SITE 12: note = misc_feature - X is 4-hydroxyproline
GFXGERGVEGPXGPA

SEQ ID NO: 228 - protein, 10 aa, synthetic construct
  REGION 1..10: note = Synthetic: Collagen
WREPSFCALS

SEQ ID NO: 229 - protein, 6 aa, synthetic construct
  REGION 1..6: note = Synthetic: Collagen
WYRGRL

SEQ ID NO: 230 - protein, 12 aa, synthetic construct
  REGION 1..12: note = Synthetic: Collagen
WTCSGDEYTWHC

SEQ ID NO: 231 - protein, 12 aa, synthetic construct
  REGION 1..12: note = Synthetic: Collagen
WTCVGDHKTWKC

SEQ ID NO: 232 - protein, 16 aa, synthetic construct
  REGION 1..16: note = Synthetic: Collagen
QWHCTTRFPHHYCLYG

SEQ ID NO: 233 - protein, 17 aa, synthetic construct
  REGION 1..17: note = Synthetic: Collagen
STWTWNGSAWTWNEGGK

SEQ ID NO: 234 - protein, 17 aa, synthetic construct
  REGION 1..17: note = Synthetic: Collagen
STWTWNGTNWTRNDGGK

SEQ ID NO: 235 - protein, 8 aa, synthetic construct
  REGION 1..8: note = Synthetic: Collagen
CVWLWEQC

SEQ ID NO: 236 - protein, 8 aa, synthetic construct
  REGION 1..8: note = Synthetic: Collagen
CMTSPWRC

SEQ ID NO: 237 - protein, 18 aa, synthetic construct
  REGION 1..18: note = Synthetic: Collagen
CPGRVMHGLHLGDDEGPC

SEQ ID NO: 238 - protein, 7 aa, synthetic construct
  REGION 1..7: note = Synthetic: Collagen
KLWLLPK

SEQ ID NO: 239 - protein, 10 aa, synthetic construct
  REGION 1..10: note = Synthetic: Collagen
CQDSETRTFY

SEQ ID NO: 240 - protein, 9 aa, synthetic construct
  REGION 1..9: note = Synthetic: Collagen
LSELRLHEN

SEQ ID NO: 241 - protein, 9 aa, synthetic construct
  REGION 1..9: note = Synthetic: Collagen
LTELHLDNN

SEQ ID NO: 242 - protein, 9 aa, synthetic construct
  REGION 1..9: note = Synthetic: Collagen
LSELRLHNN

SEQ ID NO: 243 - protein, 9 aa, synthetic construct
  REGION 1..9: note = Synthetic: Collagen
LSELRLHAN

SEQ ID NO: 244 - protein, 9 aa, synthetic construct
  REGION 1..9: note = Synthetic: Collagen
LRELHLNNN

SEQ ID NO: 245 - protein, 12 aa, synthetic construct
  REGION 1..12: note = Synthetic: Collagen
RVMHGLHLGDDE

SEQ ID NO: 246 - protein, 12 aa, synthetic construct
  REGION 1..12: note = Synthetic: Collagen
RVMHGLHLGNNQ

SEQ ID NO: 247 - protein, 12 aa, synthetic construct
  REGION 1..12: note = Synthetic: Collagen
RVMHGLHLGNNQ

SEQ ID NO: 248 - protein, 12 aa, synthetic construct
  REGION 1..12: note = Synthetic: Collagen
GQLYKSILYGSG

SEQ ID NO: 249 - protein, 11 aa, synthetic construct
  REGION 1..11: note = Synthetic: Collagen
GSGQLYKSILY

SEQ ID NO: 250 - protein, 12 aa, synthetic construct
  REGION 1..12: note = Synthetic: Collagen
GSGGQLYKSILY

SEQ ID NO: 251 - protein, 8 aa, synthetic construct
  REGION 1..8: note = Synthetic: Collagen
KQLNLVYT

SEQ ID NO: 252 - protein, 8 aa, synthetic construct
  REGION 1..8: note = Synthetic: Collagen
CVWLWQQC

SEQ ID NO: 253 - protein, 10 aa, synthetic construct
  REGION 1..10: note = Synthetic: Collagen
WREPSFSALS

SEQ ID NO: 254 - protein, 28 aa, synthetic construct
  REGION 1..28: note = Synthetic: Collagen
GHRPLDKKREEAPSLRPAPPPISGGGYR

SEQ ID NO: 255 - protein, 28 aa, synthetic construct
  REGION 1..28: note = Synthetic: Collagen
GHRPLNKKRQQAPSLRPAPPPISGGGYR

SEQ ID NO: 256 - protein, 12 aa, synthetic construct
  REGION 1..12: note = Synthetic: Collagen
GELYKSILYGSG

SEQ ID NO: 257 - protein, 12 aa, synthetic construct
  REGION 1..12: note = Synthetic: Collagen
GQLYKSILYGSG

SEQ ID NO: 258 - protein, 13 aa, synthetic construct
  REGION 1..13: note = Synthetic: Collagen
RYPISRPRKRGSG

SEQ ID NO: 259 - protein, 11 aa, synthetic construct
  REGION 1..11: note = Synthetic: Collagen
GELYKSILYGC

SEQ ID NO: 260 - protein, 11 aa, synthetic construct
  REGION 1..11: note = Synthetic: Collagen
RLDGNEIKRGC

SEQ ID NO: 261 - protein, 15 aa, synthetic construct
  REGION 1..15: note = Synthetic: Collagen
AHEEISTTNEGVMGC

SEQ ID NO: 262 - protein, 12 aa, synthetic construct
  REGION 1..12: note = Synthetic: Collagen
GCGGELYKSILY

SEQ ID NO: 263 - protein, 30 aa, synthetic construct
  REGION 1..30: note = Synthetic: Collagen
NGVFKYRPRYFLYKHAYFYPPLKRFPVQGC

SEQ ID NO: 264 - protein, 12 aa, synthetic construct
  REGION 1..12: note = Synthetic: Collagen
CQDSETRTFYGC

SEQ ID NO: 265 - protein, 9 aa, synthetic construct
  REGION 1..9: note = Synthetic: Collagen
TKKTLRTGC

SEQ ID NO: 266 - protein, 30 aa, synthetic construct
  REGION 1..30: note = Synthetic: Collagen
GLRSKSKKFRRPDIQYPDATDEDITSHMGC

SEQ ID NO: 267 - protein, 9 aa, synthetic construct
  REGION 1..9: note = Synthetic: Collagen
SQNPVQPGC

SEQ ID NO: 268 - protein, 13 aa, synthetic construct
  REGION 1..13: note = Synthetic: Collagen
SYIRIADTNITGC

SEQ ID NO: 269 - protein, 10 aa, synthetic construct
  REGION 1..10: note = Synthetic: Collagen
KELNLVYTGC

SEQ ID NO: 270 - protein, 14 aa, synthetic construct
  REGION 1..14: note = Synthetic: Collagen
GSITTIDVPWNVGC

SEQ ID NO: 271 - protein, 14 aa, synthetic construct
  REGION 1..14: note = Synthetic: Collagen
GCGGELYKSILYGC

SEQ ID NO: 272 - protein, 21 aa, synthetic construct
  REGION 1..21: note = Synthetic: Collagen
RRANAALKAGELYKSILYGSG

SEQ ID NO: 273 - protein, 8 aa, synthetic construct
  REGION 1..8: note = Synthetic: Collagen
  REGION 1..8: note = misc_feature - cyclic peptide
CVWLWENC

SEQ ID NO: 274 - protein, 8 aa, synthetic construct
  REGION 1..8: note = Synthetic: Collagen
  REGION 1..8: note = misc_feature - cyclic peptide
CVWLWEQC

SEQ ID NO: 275 - protein, 12 aa, synthetic construct
  REGION 1..12: note = Synthetic: Collagen
  REGION 1..12: note = misc_feature - D-amino acids
EDDGLHLGHMVR

SEQ ID NO: 276 - protein, 12 aa, synthetic construct
  REGION 1..12: note = Synthetic: Collagen
  REGION 1..12: note = misc_feature - D-amino acids
QNNGLHLGHMVR

SEQ ID NO: 277 - protein, 93 aa, synthetic construct
  REGION 1..93: note = Synthetic: integrin; from Fibronectin Domain III-9
PPTDLRFTNIGPDTMRVTWAPPPSIDLTNFLVRYSPVKNEEDVAELSISPSDNAVVLTNLLPGTEYVVSVSSVYEQHESTPLRGRQKTGLDSP

SEQ ID NO: 278 - protein, 88 aa, synthetic construct
  REGION 1..88: note = Synthetic: integrin; from Fibronectin Domain III-10
TGIDFSDITANSFTVHWIAPRATITGYRIRHHPEHFSGRPREDRVPHSRNSITLTNLTPGTEYVVSIVALNGREESPLLIGQQSTVSD

SEQ ID NO: 279 - protein, 41 aa, synthetic construct
  REGION 1..41: note = Synthetic: integrin; from Fibronectin Domain 1-1
PGCYDNGKHYQINQQWERTYLGNALVCTCYGGSRGFNCESK

SEQ ID NO: 280 - protein, 44 aa, synthetic construct
  REGION 1..44: note = Synthetic: integrin; from Fibronectin Domain 1-2
ETCFDKYTGNTYRVGDTYERPKDSMIWDCTCIGAGRGRISCTIA

SEQ ID NO: 281 - protein, 44 aa, synthetic construct
  REGION 1..44: note = Synthetic: integrin; from Fibronectin Domain 1-3
NRCHEGGQSYKIGDTWRRPHETGGYMLECVCLGNGKGEWTCKPI

SEQ ID NO: 282 - protein, 45 aa, synthetic construct
  REGION 1..45: note = Synthetic: integrin; from Fibronectin Domain 1-4
EKCFDHAAGTSYVVGETWEKPYQGWMMVDCTCLGEGSGRITCTSR

SEQ ID NO: 283 - protein, 45 aa, synthetic construct
  REGION 1..45: note = Synthetic: integrin; from Fibronectin Domain 1-5
NRCNDQDTRTSYRIGDTWSKKDNRGNLLQCICTGNGRGEWKCERH

SEQ ID NO: 284 - protein, 40 aa, synthetic construct; identical to SEQ ID NO: 182
  REGION 1..40: note = Synthetic: denatured Collagen / integrin; from Fibronectin Domain 1-6

SEQ ID NO: 285 - protein, 44 aa, synthetic construct; identical to SEQ ID NO: 183
  REGION 1..44: note = Synthetic: denatured Collagen / integrin; from Fibronectin Domain 1-7

SEQ ID NO: 286 - protein, 43 aa, synthetic construct; identical to SEQ ID NO: 184
  REGION 1..43: note = Synthetic: denatured Collagen / integrin; from Fibronectin Domain 1-8

SEQ ID NO: 287 - protein, 44 aa, synthetic construct; identical to SEQ ID NO: 185
  REGION 1..44: note = Synthetic: denatured Collagen / integrin; from Fibronectin Domain 1-9

SEQ ID NO: 288 - protein, 94 aa, synthetic construct
  REGION 1..94: note = Synthetic: integrin; from Fibronectin Domain III-14
APTDLKFTQVTPTSLSAQWTPPNVQLTGYRVRVTPKEKTGPMKEINLAPDSSSVVVSGLMVATKYEVSVYALKDTLTSRPAQGVVTTLENVSPP

SEQ ID NO: 289 - protein, 90 aa, synthetic construct
  REGION 1..90: note = Synthetic: integrin; from Fibronectin Domain III-5
APTNLQFVNETDSTVLVRWTPPRAQITGYRLTVGLTRRGQPRQYNVGPSVSKYPLRNLQPASEYTVSLVAIKGNQESPKATGVFTTLQPG

SEQ ID NO: 290 - protein, 6 aa, synthetic construct
  REGION 1..6: note = Synthetic: integrin; Derived from Collagen I
KGHRGF

SEQ ID NO: 291 - protein, 6 aa, synthetic construct
  REGION 1..6: note = Synthetic: integrin; Derived from Collagen I
GFPGER

SEQ ID NO: 292 - protein, 15 aa, synthetic construct
  REGION 1..15: note = Synthetic: integrin; Derived from Collagen alpha1(I)
GTPGPQGIAGQRDVV

SEQ ID NO: 293 - protein, 5 aa, synthetic construct
  REGION 1..5: note = Synthetic: integrin; Derived from Collagen II
EKGPD

SEQ ID NO: 294 - protein, 6 aa, synthetic construct
  REGION 1..6: note = Synthetic: integrin; Derived from Collagen II
EKGPDP

SEQ ID NO: 295 - protein, 7 aa, synthetic construct
  REGION 1..7: note = Synthetic: integrin; Derived from Collagen II
EKGPDPL

SEQ ID NO: 296 - protein, 12 aa, synthetic construct
  REGION 1..12: note = Synthetic: integrin; Derived from Collagen Iv
TAGSCLRKFSTM

SEQ ID NO: 297 - protein, 15 aa, synthetic construct
  REGION 1..15: note = Synthetic: integrin; Derived from Collagen alpha3(Iv)-NC1
TAIPSCPEGTVPLYS

SEQ ID NO: 298 - protein, 15 aa, synthetic construct
  REGION 1..15: note = Synthetic: integrin; Derived from Collagen Iv
TDIPPCPHGWISLWK

SEQ ID NO: 299 - protein, 5 aa, synthetic construct
  REGION 1..5: note = Synthetic: integrin; Derived from Fibronectin
PHSRN

SEQ ID NO: 300 - skipped

SEQ ID NO: 301 - protein, 6 aa, synthetic construct
  REGION 1..6: note = Synthetic: integrin; Derived from Fibronectin
GRGDSP

SEQ ID NO: 302 - protein, 16 aa, synthetic construct
  REGION 1..16: note = Synthetic: integrin; Derived from Fibronectin
YRVRVTPKEKTGPMKE

SEQ ID NO: 303 - protein, 9 aa, synthetic construct
  REGION 1..9: note = Synthetic: integrin; Derived from Fibronectin
SPPRRARVT

SEQ ID NO: 304 - protein, 8 aa, synthetic construct
  REGION 1..8: note = Synthetic: integrin; Derived from Fibronectin
WQPPRARI

SEQ ID NO: 305 - protein, 15 aa, synthetic construct
  REGION 1..15: note = Synthetic: integrin; Derived from Fibronectin
KNNQKSEPLIGRKKT

SEQ ID NO: 306 - protein, 8 aa, synthetic construct
  REGION 1..8: note = Synthetic: integrin; Derived from Fibronectin
EILDVPST

SEQ ID NO: 307 - protein, 4 aa, synthetic construct
  REGION 1..4: note = Synthetic: integrin; Derived from Fibronectin
REDV

SEQ ID NO: 308 - protein, 13 aa, synthetic construct
  REGION 1..13: note = Synthetic: integrin; Derived from Laminin Alpha-1 chain
RQVFQVAYIIIKA

SEQ ID NO: 309 - protein, 12 aa, synthetic construct
  REGION 1..12: note = Synthetic: integrin; Derived from Laminin Alpha-1 chain
SINNTAVMQRLT

SEQ ID NO: 310 - protein, 5 aa, synthetic construct
  REGION 1..5: note = Synthetic: integrin; Derived from Laminin Alpha-1 chain
IKVAV

SEQ ID NO: 311 - protein, 12 aa, synthetic construct
  REGION 1..12: note = Synthetic: integrin; Derived from Laminin Alpha-1 chain
NRWHSIYITRFG

SEQ ID NO: 312 - protein, 12 aa, synthetic construct
  REGION 1..12: note = Synthetic: integrin; Derived from Laminin Alpha-1 chain
TWYKIAFQRNRK

SEQ ID NO: 313 - protein, 12 aa, synthetic construct
  REGION 1..12: note = Synthetic: integrin; Derived from Laminin Alpha-1 chain
RKRLQVQLSIRT

SEQ ID NO: 314 - protein, 12 aa, synthetic construct
  REGION 1..12: note = Synthetic: integrin; Derived from Laminin Alpha-2 chain
KNRLTIELEVRT

SEQ ID NO: 315 - protein, 12 aa, synthetic construct
  REGION 1..12: note = Synthetic: integrin; Derived from Laminin Alpha-2 chain
SYWYRIEASRTG

SEQ ID NO: 316 - protein, 19 aa, synthetic construct
  REGION 1..19: note = Synthetic: integrin; Derived from Laminin Alpha-2 chain
DFGTVQLRNGFPFFSYDLG

SEQ ID NO: 317 - protein, 13 aa, synthetic construct
  REGION 1..13: note = Synthetic: integrin; Derived from Laminin Alpha-3 chain
GQLFHVAYILIKF

SEQ ID NO: 318 - protein, 12 aa, synthetic construct
  REGION 1..12: note = Synthetic: integrin; Derived from Laminin Alpha-3 chain
KNSFMALYLSKG

SEQ ID NO: 319 - protein, 12 aa, synthetic construct
  REGION 1..12: note = Synthetic: integrin; Derived from Laminin Alpha-4 chain
TLFLAHGRLVFM

SEQ ID NO: 320 - protein, 13 aa, synthetic construct
  REGION 1..13: note = Synthetic: integrin; Derived from Laminin Alpha-5 chain
GQVFHVAYVLIKF

SEQ ID NO: 321 - protein, 6 aa, synthetic construct
  REGION 1..6: note = Synthetic: integrin; Derived from Laminin Alpha-5 chain
GIIFFL

SEQ ID NO: 322 - protein, 12 aa, synthetic construct
  REGION 1..12: note = Synthetic: integrin; Derived from Laminin Alpha-5 chain
LALFLSNGHFVA

SEQ ID NO: 323 - protein, 7 aa, synthetic construct
  REGION 1..7: note = Synthetic: integrin; Derived from Laminin Beta-1 chain
RYVVLPR

SEQ ID NO: 324 - protein, 5 aa, synthetic construct
  REGION 1..5: note = Synthetic: integrin; Derived from Laminin Beta-1 chain
PDSGR

SEQ ID NO: 325 - protein, 5 aa, synthetic construct
  REGION 1..5: note = Synthetic: integrin; Derived from Laminin Beta-1 chain
YIGSR

SEQ ID NO: 326 - protein, 12 aa, synthetic construct
  REGION 1..12: note = Synthetic: integrin; Derived from Laminin Gamma-1 chain
KAFDITYVRLKF

SEQ ID NO: 327 - protein, 10 aa, synthetic construct
  REGION 1..10: note = Synthetic: integrin; Derived from Laminin Gamma-1 chain
RNIAEIIKDI

SEQ ID NO: 328 - protein, 9 aa, synthetic construct
  REGION 1..9: note = Synthetic: integrin; Derived from vitronectin
FRHRNRKGY

SEQ ID NO: 329 - protein, 16 aa, synthetic construct
  REGION 1..16: note = Synthetic: integrin; Derived from vitronectin
KKQRFRHRNRKGYRSQ

SEQ ID NO: 330 - protein, 7 aa, synthetic construct
  REGION 1..7: note = Synthetic: integrin; Derived from Sialoprotein
FHRRIKA

SEQ ID NO: 331 - protein, 4 aa, synthetic construct
  REGION 1..4: note = Synthetic: integrin; Derived from Sialoprotein
KRSR

SEQ ID NO: 332 - protein, 6 aa, synthetic construct
  REGION 1..6: note = Synthetic: alpha-1-beta-1, alpha-2-beta-1; Derived from Collagen alpha-1(I) 7S
GLPGER

SEQ ID NO: 333 - protein, 6 aa, synthetic construct
  REGION 1..6: note = Synthetic: alpha-1-beta-1, alpha-2-beta-1; Derived from Collagen alpha1(I)
GFPGER

SEQ ID NO: 334 - protein, 6 aa, synthetic construct
  REGION 1..6: note = Synthetic: alpha-2-beta-1; Derived from Collagen alpha1(I)
GLSGER

SEQ ID NO: 335 - protein, 4 aa, synthetic construct
  REGION 1..4: note = Synthetic: alpha-2-beta-1; Derived from Collagen alpha1(I)
DGEA

SEQ ID NO: 336 - protein, 13 aa, synthetic construct
  REGION 1..13: note = Synthetic: alpha-2-beta-1; Derived from Collagen alpha1(I)
GPAGKDGEAGAQG

SEQ ID NO: 337 - protein, 12 aa, synthetic construct
  REGION 1..12: note = Synthetic: alpha-1-beta-1, alpha-3-beta-1; Derived from Collagen alpha1(I)
GPKGAAGEPGKP

SEQ ID NO: 338 - protein, 12 aa, synthetic construct
  REGION 1..12: note = Synthetic: alpha-1-beta-1, alpha-3-beta-1; Derived from Collagen alpha1(I)
GAPGPKGARGSA

SEQ ID NO: 339 - protein, 15 aa, synthetic construct
  REGION 1..15: note = Synthetic: alpha-1-beta-1; Derived from Collagen alpha1(I)
GPQGIAGQRGVVGLP

SEQ ID NO: 340 - protein, 9 aa, synthetic construct
  REGION 1..9: note = Synthetic: Poly(I); Derived from Collagen alpha1(I)
PKGQKGEKG

SEQ ID NO: 341 - protein, 6 aa, synthetic construct
  REGION 1..6: note = Synthetic: alpha-2-beta-1; Derived from Collagen alpha1(I)
GASGER

SEQ ID NO: 342 - protein, 6 aa, synthetic construct
  REGION 1..6: note = Synthetic: alpha-2-beta-1; Derived from Collagen alpha1(I)
GQRGER

SEQ ID NO: 343 - protein, 6 aa, synthetic construct
  REGION 1..6: note = Synthetic: integrin; Derived from Collagen alpha1(I)
GMPGER

SEQ ID NO: 344 - protein, 9 aa, synthetic construct
  REGION 1..9: note = Synthetic: vWF; Derived from Collagen alpha1(III)
RGQPGVMGF

SEQ ID NO: 345 - protein, 6 aa, synthetic construct
  REGION 1..6: note = Synthetic: alpha-2-beta-1; Derived from Collagen alpha1(III)
GKDGES

SEQ ID NO: 346 - protein, 6 aa, synthetic construct
  REGION 1..6: note = Synthetic: alpha-2-beta-1; Derived from Collagen alpha1(III)
GLKGEN

SEQ ID NO: 347 - protein, 6 aa, synthetic construct
  REGION 1..6: note = Synthetic: alpha-2-beta-1; Derived from Collagen alpha1(III)
GLPGEN

SEQ ID NO: 348 - protein, 6 aa, synthetic construct
  REGION 1..6: note = Synthetic: alpha-2-beta-1; Derived from Collagen alpha1(III)
GLPGEA

SEQ ID NO: 349 - protein, 12 aa, synthetic construct
  REGION 1..12: note = Synthetic: alpha-1-beta-1; Derived from Collagen alpha1(Iv)
GPPGDQGPPGIP

SEQ ID NO: 350 - protein, 12 aa, synthetic construct
  REGION 1..12: note = Synthetic: alpha-1-beta-1; Derived from Collagen alpha1(Iv)
GAKGRAGFPGLP

SEQ ID NO: 351 - protein, 16 aa, synthetic construct
  REGION 1..16: note = Synthetic: integrin; Derived from Collagen alpha1(Iv)
MFKKPTPSTLKAGELR

SEQ ID NO: 352 - protein, 12 aa, synthetic construct
  REGION 1..12: note = Synthetic: integrin; Derived from Collagen alpha1(Iv)
GFPGSRGDTGPP

SEQ ID NO: 353 - protein, 15 aa, synthetic construct
  REGION 1..15: note = Synthetic: integrin; Derived from Collagen alpha1(Iv)
GVKGDKGNPGWPGAP

SEQ ID NO: 354 - protein, 6 aa, synthetic construct
  REGION 1..6: note = Synthetic: alpha-1-beta-1, alpha-2-beta-1; Derived from Collagen alpha1(Iv)
FYFDLR

SEQ ID NO: 355 - protein, 16 aa, synthetic construct
  REGION 1..16: note = Synthetic: integrin; Derived from Collagen alpha1(Iv)
MFKKPTPSTLKAGELR

SEQ ID NO: 356 - protein, 12 aa, synthetic construct
  REGION 1..12: note = Synthetic: integrin; Derived from Collagen alpha1(Iv)
GFPGSRGDTGPP

SEQ ID NO: 357 - protein, 15 aa, synthetic construct
  REGION 1..15: note = Synthetic: integrin; Derived from Collagen alpha1(Iv)
GVKGDKGNPGWPGAP

SEQ ID NO: 358 - protein, 6 aa, synthetic construct
  REGION 1..6: note = Synthetic: alpha-1-beta-1, alpha-2-beta-1; Derived from Collagen alpha1(Iv)
FYFDLR

SEQ ID NO: 359 - protein, 15 aa, synthetic construct
  REGION 1..15: note = Synthetic: integrin; Derived from Collagen alpha2(Iv)
RGQPGVPGVPGMKGD

SEQ ID NO: 360 - protein, 15 aa, synthetic construct
  REGION 1..15: note = Synthetic: integrin; Derived from Collagen alpha3(Iv)-NC1
TDIPPCPHGWISLWK

SEQ ID NO: 361 - protein, 7 aa, synthetic construct
  REGION 1..7: note = Synthetic: integrin; Derived from Collagen alpha3(Iv)-NC1
MNYYSNS

SEQ ID NO: 362 - protein, 19 aa, synthetic construct
  REGION 1..19: note = Synthetic: integrin; Derived from Collagen alpha3(Iv)-NC1
CNYYSNSYSFWLASLNPER

SEQ ID NO: 363 - protein, 12 aa, synthetic construct
  REGION 1..12: note = Synthetic: integrin; Derived from Collagen alpha3(Iv)-NC1
ISRCQVCMKKRH

SEQ ID NO: 364 - protein, 12 aa, synthetic construct
  REGION 1..12: note = Synthetic: integrin; Derived from Collagen alpha3(Iv)-NC1
TLGSCLQRFTTM

SEQ ID NO: 365 - protein, 6 aa, synthetic construct
  REGION 1..6: note = Synthetic: integrin; Derived from Collagen alpha3(Iv)-NC1
GRRGKT

SEQ ID NO: 366 - protein, 9 aa, synthetic construct
  REGION 1..9: note = Synthetic: integrin; Derived from Collagen alpha3(Iv)-NC1
RGQPGRKGL

SEQ ID NO: 367 - protein, 12 aa, synthetic construct
  REGION 1..12: note = Synthetic: integrin; Derived from Collagen alpha3(Iv)-NC1
MFRKPIPSTVKA

SEQ ID NO: 368 - protein, 13 aa, synthetic construct
  REGION 1..13: note = Synthetic: integrin; Derived from Collagen alpha3(Iv)-NC1
IISRCQVCMKMRP

SEQ ID NO: 369 - protein, 12 aa, synthetic construct
  REGION 1..12: note = Synthetic: integrin; Derived from Collagen alpha4(Iv)-NC1
LAGSCLPVFSTL

SEQ ID NO: 370 - protein, 12 aa, synthetic construct
  REGION 1..12: note = Synthetic: integrin; Derived from Collagen alpha5(Iv)-NC1
TAGSCLRRFSTM

SEQ ID NO: 371 - protein, 6 aa, synthetic construct
  REGION 1..6: note = Synthetic: integrin; Derived from Collagen alpha5(Iv)-NC2
NKRAHG

SEQ ID NO: 372 - protein, 20 aa, synthetic construct
  REGION 1..20: note = Synthetic: alpha-5-beta-1; Derived from Fibronectin III-5
WTPPRAQITGYRLTVGLTRR

SEQ ID NO: 373 - protein, 6 aa, synthetic construct
  REGION 1..6: note = Synthetic: alpha-4-beta-1, alpha-4-beta-7; Derived from FibronectinIII-5
KLDAPT

SEQ ID NO: 374 - protein, 5 aa, synthetic construct
  REGION 1..5: note = Synthetic: alpha-5-beta-1; Derived from FibronectinIII-9
PHSRN

SEQ ID NO: 375 - skipped

SEQ ID NO: 376 - protein, 4 aa, synthetic construct
  REGION 1..4: note = Synthetic: alpha-IIb-beta-3; Derived from FibronectinIII-10
RGDS

SEQ ID NO: 377 - protein, 6 aa, synthetic construct
  REGION 1..6: note = Synthetic: alpha-5-beta-1; Derived from FibronectinIII-10
GRGDSP

SEQ ID NO: 378 - protein, 7 aa, synthetic construct
  REGION 1..7: note = Synthetic: alpha-4-beta-1, alpha-9-beta-1; Derived from Fibronectin EDA
EDGIHEL

SEQ ID NO: 379 - protein, 30 aa, synthetic construct
  REGION 1..30: note = Synthetic: integrin; Derived from FibronectinIII-14
PRARITGYIIKYEKPGSPPREVVPRPRPGV

SEQ ID NO: 380 - protein, 5 aa, synthetic construct
  REGION 1..5: note = Synthetic: alpha-4-beta-1; Derived from Fibronectin IIICS-1
IDAPS

SEQ ID NO: 381 - protein, 15 aa, synthetic construct
  REGION 1..15: note = Synthetic: alpha-4-beta-1; Derived from Fibronectin IIICS-1
VVIDASTAIDAPSNL

SEQ ID NO: 382 - protein, 5 aa, synthetic construct
  REGION 1..5: note = Synthetic: alpha-4-beta-1; Derived from Fibronectin IIICS-1
LDVPS

SEQ ID NO: 383 - protein, 4 aa, synthetic construct
  REGION 1..4: note = Synthetic: alpha-4-beta-1; Derived from Fibronectin IIICS-5
REDV

SEQ ID NO: 384 - protein, 10 aa, synthetic construct
  REGION 1..10: note = Synthetic: alpha-5-beta-1; Derived from Fibronectin III-10
PHSRNRGDSP

SEQ ID NO: 385 - protein, 10 aa, synthetic construct
  REGION 1..10: note = Synthetic: integrin; Derived from E-Cadherin EC1
PLDREAIAKY

SEQ ID NO: 386 - protein, 5 aa, synthetic construct
  REGION 1..5: note = Synthetic: integrin; Derived from E-Cadherin EC1, groove
HAVDI

SEQ ID NO: 387 - protein, 10 aa, synthetic construct
  REGION 1..10: note = Synthetic: integrin; Derived from E-Cadherin EC1, groove
LFSHAVSSNG

SEQ ID NO: 388 - protein, 6 aa, synthetic construct
  REGION 1..6: note = Synthetic: integrin; Derived from E-Cadherin EC1, bulge
ADTPPV

SEQ ID NO: 389 - protein, 10 aa, synthetic construct
  REGION 1..10: note = Synthetic: integrin; Derived from E-Cadherin EC1, bulge
QGADTPPVGV

SEQ ID NO: 390 - protein, 10 aa, synthetic construct
  REGION 1..10: note = Synthetic: integrin; Derived from E-Cadherin EC1
PLDREAIAKY

SEQ ID NO: 391 - protein, 5 aa, synthetic construct
  REGION 1..5: note = Synthetic: integrin; Derived from E-Cadherin EC1
DQNDN

SEQ ID NO: 392 - protein, 5 aa, synthetic construct
  REGION 1..5: note = Synthetic: integrin; Derived from E-Cadherin EC1
HAVDI

SEQ ID NO: 393 - protein, 10 aa, synthetic construct
  REGION 1..10: note = Synthetic: integrin; Derived from E-Cadherin EC1
LRAHAVDING

SEQ ID NO: 394 - protein, 10 aa, synthetic construct
  REGION 1..10: note = Synthetic: integrin; Derived from E-Cadherin EC1
LRAHAVDVNG

SEQ ID NO: 395 - protein, 10 aa, synthetic construct
  REGION 1..10: note = Synthetic: integrin; Derived from E-Cadherin EC2
VITVKDINDN

SEQ ID NO: 396 - protein, 10 aa, synthetic construct
  REGION 1..10: note = Synthetic: integrin; Derived from E-Cadherin EC2
GLDRESYPYY

SEQ ID NO: 397 - protein, 10 aa, synthetic construct
  REGION 1..10: note = Synthetic: integrin; Derived from E-Cadherin EC2
MKVSATDADD

SEQ ID NO: 398 - protein, 10 aa, synthetic construct
  REGION 1..10: note = Synthetic: integrin; Derived from E-Cadherin EC2, bulge
QDPELPDKNM

SEQ ID NO: 399 - protein, 10 aa, synthetic construct
  REGION 1..10: note = Synthetic: integrin; Derived from E-Cadherin EC2, groove
LVVQAADLQG

SEQ ID NO: 400 - protein, 10 aa, synthetic construct
  REGION 1..10: note = Synthetic: integrin; Derived from E-Cadherin EC3, bulge
NDDGGQFVVT

SEQ ID NO: 401 - protein, 10 aa, synthetic construct
  REGION 1..10: note = Synthetic: integrin; Derived from E-Cadherin EC2, groove
LVVQAADLQG

SEQ ID NO: 402 - protein, 10 aa, synthetic construct
  REGION 1..10: note = Synthetic: integrin; Derived from E-Cadherin EC4, bulge
TYRIWRDTAN

SEQ ID NO: 403 - protein, 10 aa, synthetic construct
  REGION 1..10: note = Synthetic: integrin; Derived from E-Cadherin EC3, groove
YILHVAVTNY

SEQ ID NO: 404 - protein, 10 aa, synthetic construct
  REGION 1..10: note = Synthetic: integrin; Derived from E-Cadherin EC4, groove
YTALIIATDN

SEQ ID NO: 405 - protein, 10 aa, synthetic construct
  REGION 1..10: note = Synthetic: integrin; Derived from E-Cadherin EC2, bulge
QDPELPDKNM

SEQ ID NO: 406 - protein, 4 aa, synthetic construct
  REGION 1..4: note = Synthetic: alpha-v-beta-3, alpha-v-beta-5; Somatomedin B
RGDV

SEQ ID NO: 407 - protein, 12 aa, synthetic construct
  REGION 1..12: note = Synthetic: alpha-v-beta-3, alpha-v-beta-5; Somatomedin B
PQVTRGDVFTMP

SEQ ID NO: 408 - protein, 10 aa, synthetic construct
  REGION 1..10: note = Synthetic: integrin; Nidogen G2
LNRQELFPFG

SEQ ID NO: 409 - protein, 11 aa, synthetic construct
  REGION 1..11: note = Synthetic: integrin; Nidogen G2
SIGFRGDGQTC

SEQ ID NO: 410 - protein, 17 aa, synthetic construct
  REGION 1..17: note = Synthetic: IgB; Perlecan Iv
TWSKVGGHLRPGIVQSG

SEQ ID NO: 411 - protein, 9 aa, synthetic construct
  REGION 1..9: note = Synthetic: alpha-9-beta-1; Tenascin-C
VAEIDGIEL

SEQ ID NO: 412 - protein, 8 aa, synthetic construct
  REGION 1..8: note = Synthetic: alpha-7-beta-1; Tenascin-C
VFDNFVLK

SEQ ID NO: 413 - protein, 6 aa, synthetic construct
  REGION 1..6: note = Synthetic: integrin; Elastin
VGVAPG

SEQ ID NO: 414 - protein, 5 aa, synthetic construct
  REGION 1..5: note = Synthetic: integrin; Elastin
PGVGV

SEQ ID NO: 415 - protein, 10 aa, synthetic construct
  REGION 1..10: note = Synthetic: alpha-6-beta-1; CCN-1
TTSWSQCSKS

SEQ ID NO: 416 - protein, 7 aa, synthetic construct
  REGION 1..7: note = Synthetic: alpha-9-beta-1; Osteopontin
SVVYGLR

SEQ ID NO: 417 - protein, 10 aa, synthetic construct
  REGION 1..10: note = Synthetic: alpha-v-beta-3; Osteopontin
DGRGDSVAYG

SEQ ID NO: 418 - protein, 10 aa, synthetic construct
  REGION 1..10: note = Synthetic: alpha-6-beta-1; Thrombospondin
LALERKDHSG

SEQ ID NO: 419 - protein, 4 aa, synthetic construct
  REGION 1..4: note = Synthetic: alpha-IIIb-beta-3; Fibrinogen
RGDF

SEQ ID NO: 420 - protein, 7 aa, synthetic construct
  REGION 1..7: note = Synthetic: alpha-M-beta-2; Fibrinogen
KRLDGSV

SEQ ID NO: 421 - protein, 12 aa, synthetic construct
  REGION 1..12: note = Synthetic: alpha-IIb-beta-3; Fibrinogen
HHLGGAKQAGDV

SEQ ID NO: 422 - protein, 19 aa, synthetic construct
  REGION 1..19: note = Synthetic: alpha-IIb-beta-3; Fibrinogen
YSMKKTTMKIIPFNRLTIG

SEQ ID NO: 423 - protein, 13 aa, synthetic construct
  REGION 1..13: note = Synthetic: alpha-M-beta-2; Fibrinogen
GVYYQGGTYSKAS

SEQ ID NO: 424 - protein, 13 aa, synthetic construct
  REGION 1..13: note = Synthetic: alpha-5-beta-1; Laminin alpha-1 LN (A3)
LWVTVRSQQRGLF

SEQ ID NO: 425 - protein, 13 aa, synthetic construct
  REGION 1..13: note = Synthetic: alpha-4-beta-1, alpha-4-beta-7; Laminin alpha-1 LN (A10)
GTNNWWQSPSIQN

SEQ ID NO: 426 - protein, 13 aa, synthetic construct
  REGION 1..13: note = Synthetic: alpha-5-beta-1; Laminin alpha-1 LN (A12)
WVTVTLDLRQVFQ

SEQ ID NO: 427 - protein, 13 aa, synthetic construct
  REGION 1..13: note = Synthetic: alpha-1-beta-1, alpha-2-beta-1; Laminin alpha-1 LN (A13)
RQVFQVAYIIIKA

SEQ ID NO: 428 - protein, 14 aa, synthetic construct
  REGION 1..14: note = Synthetic: alpha-5-beta-1; Laminin alpha-1 LN (A18)
LTRYKITPRRGPPT

SEQ ID NO: 429 - protein, 12 aa, synthetic construct
  REGION 1..12: note = Synthetic: integrin; LamininLaminin alpha-1 LN (A24)
LLEFTSARYIRL

SEQ ID NO: 430 - protein, 12 aa, synthetic construct
  REGION 1..12: note = Synthetic: integrin; Laminin alpha-1 LN (A25)
YIRLRLQRIRTL

SEQ ID NO: 431 - protein, 12 aa, synthetic construct
  REGION 1..12: note = Synthetic: integrin; Lamininalpha-1 v? (A29)
RRYYYSIKDISV

SEQ ID NO: 432 - protein, 12 aa, synthetic construct
  REGION 1..12: note = Synthetic: integrin; Laminin alpha-1 L4a (A55)
GGFLKYTVSYDI

SEQ ID NO: 433 - protein, 12 aa, synthetic construct
  REGION 1..12: note = Synthetic: integrin; Laminin alpha-1 L4a (A64)
RDQLMTVLANVT

SEQ ID NO: 434 - protein, 12 aa, synthetic construct
  REGION 1..12: note = Synthetic: alpha-5-beta-1; Laminin alpha-1 L4a (A112)
VLIKGGRARKHV

SEQ ID NO: 435 - protein, 12 aa, synthetic construct
  REGION 1..12: note = Synthetic: integrin; Laminin alpha-1 L1 (A167)
NLLLLLVKANLK

SEQ ID NO: 436 - protein, 12 aa, synthetic construct
  REGION 1..12: note = Synthetic: integrin; Laminin alpha-1 L1 (A174)
HRDELLLWARKI

SEQ ID NO: 437 - protein, 12 aa, synthetic construct
  REGION 1..12: note = Synthetic: integrin; Laminin alpha-1 L1 (A177)
KRRARDLVHRAE

SEQ ID NO: 438 - protein, 12 aa, synthetic construct
  REGION 1..12: note = Synthetic: integrin; Laminin alpha-1 L1 (A191)
SQFQESVDNITK

SEQ ID NO: 439 - protein, 12 aa, synthetic construct
  REGION 1..12: note = Synthetic: integrin; Laminin alpha-1 L1 (A194)
PGGMREKGRKAR

SEQ ID NO: 440 - protein, 12 aa, synthetic construct
  REGION 1..12: note = Synthetic: integrin; Laminin alpha-1 L1 (A203)
MEMQANLLLDRL

SEQ ID NO: 441 - protein, 12 aa, synthetic construct
  REGION 1..12: note = Synthetic: integrin; Laminin alpha-1 L1 (A206)
LSEIKLLISRAR

SEQ ID NO: 442 - protein, 5 aa, synthetic construct
  REGION 1..5: note = Synthetic: alpha-v-beta-3; Laminin alpha-1 L1 (A208)
IKVAV

SEQ ID NO: 443 - protein, 12 aa, synthetic construct
  REGION 1..12: note = Synthetic: alpha-v-beta-3; Laminin alpha-1 L1 (A208)
AASIKVAVSADR

SEQ ID NO: 444 - protein, 12 aa, synthetic construct
  REGION 1..12: note = Synthetic: alpha-6-beta-1; Laminin alpha-1 LG1 (AG10)
NRWHSIYITRFG

SEQ ID NO: 445 - protein, 12 aa, synthetic construct
  REGION 1..12: note = Synthetic: integrin; Laminin alpha-1 LG2 (AG22)
SSFHFDGSGYAM

SEQ ID NO: 446 - protein, 6 aa, synthetic construct
  REGION 1..6: note = Synthetic: alpha-6-beta-1; Laminin alpha-1 LG2 (AG32)
IAFQRN

SEQ ID NO: 447 - protein, 12 aa, synthetic construct
  REGION 1..12: note = Synthetic: alpha-6-beta-1; Laminin alpha-1 LG2 (AG32)
TWYKIAFQRNRK

SEQ ID NO: 448 - protein, 12 aa, synthetic construct
  REGION 1..12: note = Synthetic: integrin; Laminin alpha-1 LG2 (AG56)
SLVRNRRVITIQ

SEQ ID NO: 449 - protein, 19 aa, synthetic construct
  REGION 1..19: note = Synthetic: alpha-2-beta-1; Laminin EF-1
DYATLQLQEGRLHFMFDLG

SEQ ID NO: 450 - protein, 12 aa, synthetic construct
  REGION 1..12: note = Synthetic: integrin; Laminin alpha-2 LG (A2G2)
KKGSYNNIVVHV

SEQ ID NO: 451 - protein, 12 aa, synthetic construct
  REGION 1..12: note = Synthetic: integrin; Laminin alpha-2 LG (A2G4)
ADNLLFYLGSAK

SEQ ID NO: 452 - protein, 12 aa, synthetic construct
  REGION 1..12: note = Synthetic: integrin; Laminin alpha-2 LG (A2G5)
GSAKFIDFLAIE

SEQ ID NO: 453 - protein, 12 aa, synthetic construct
  REGION 1..12: note = Synthetic: integrin; Laminin alpha-2 LG (A2G7)
KVSFLWWVGSGV

SEQ ID NO: 454 - protein, 12 aa, synthetic construct
  REGION 1..12: note = Synthetic: integrin; Laminin alpha-2 LG (A2G10)
SYWYRIEASRTG

SEQ ID NO: 455 - protein, 12 aa, synthetic construct
  REGION 1..12: note = Synthetic: integrin; Laminin alpha-2 LG (A2G25)
ISTVMFKFRTFS

SEQ ID NO: 456 - protein, 13 aa, synthetic construct
  REGION 1..13: note = Synthetic: integrin; Laminin alpha-2 LG (A2G34)
KQANISIVDIDSN

SEQ ID NO: 457 - protein, 12 aa, synthetic construct
  REGION 1..12: note = Synthetic: integrin; Laminin alpha-2 LG (A2G48)
FSTRNESGIILL

SEQ ID NO: 458 - protein, 12 aa, synthetic construct
  REGION 1..12: note = Synthetic: integrin; Laminin alpha-2 LG (A2G51)
RRQTTQAYYAIF

SEQ ID NO: 459 - protein, 12 aa, synthetic construct
  REGION 1..12: note = Synthetic: integrin; Laminin alpha-2 LG (A2G52)
YAIFLNKGRLEV

SEQ ID NO: 460 - protein, 12 aa, synthetic construct
  REGION 1..12: note = Synthetic: integrin; Laminin alpha-2 LG (A2G76)
KNRLTIELEVRT

SEQ ID NO: 461 - protein, 12 aa, synthetic construct
  REGION 1..12: note = Synthetic: integrin; Laminin alpha-2 LG (A2G78)
GLLFYMARINHA

SEQ ID NO: 462 - protein, 12 aa, synthetic construct
  REGION 1..12: note = Synthetic: integrin; Laminin alpha-2 LG (A2G80)
VQLRNGFPYFSY

SEQ ID NO: 463 - protein, 12 aa, synthetic construct
  REGION 1..12: note = Synthetic: integrin; Laminin alpha-2 LG (A2G84)
HKIKIVRVKQEG

SEQ ID NO: 464 - protein, 19 aa, synthetic construct
  REGION 1..19: note = Synthetic: integrin; Laminin EF-2
DFGTVQLRNGFPFFSYDLG

SEQ ID NO: 465 - protein, 12 aa, synthetic construct
  REGION 1..12: note = Synthetic: integrin; Laminin alpha-2 LG (A2G94)
YFDGTGFAKAVG

SEQ ID NO: 466 - protein, 12 aa, synthetic construct
  REGION 1..12: note = Synthetic: integrin; Laminin alpha-2 LG (A2G103)
NGQWHKVTAKKI

SEQ ID NO: 467 - protein, 12 aa, synthetic construct
  REGION 1..12: note = Synthetic: integrin; Laminin alpha-2 LG (A2G104)
AKKIKNRLELVV

SEQ ID NO: 468 - protein, 14 aa, synthetic construct
  REGION 1..14: note = Synthetic: integrin; Laminin alpha-2 LG (A2G109)
GFPGGLNQFGLTTN

SEQ ID NO: 469 - protein, 13 aa, synthetic construct
  REGION 1..13: note = Synthetic: integrin; Laminin alpha-2 LG (A2G111)
IRSLKLTKGTGKP

SEQ ID NO: 470 - protein, 13 aa, synthetic construct
  REGION 1..13: note = Synthetic: integrin; Laminin alpha-2 LG (A2G113)
AKALELRGVQPVS

SEQ ID NO: 471 - protein, 13 aa, synthetic construct
  REGION 1..13: note = Synthetic: integrin; Laminin alpha-3 (A3-10)
GQLFHVAYILIKF

SEQ ID NO: 472 - protein, 13 aa, synthetic construct
  REGION 1..13: note = Synthetic: integrin; Laminin alpha-3 LG (MA3G13)
SQRIYQFAKLNYT

SEQ ID NO: 473 - protein, 12 aa, synthetic construct
  REGION 1..12: note = Synthetic: integrin; Laminin alpha-3 LG (MA3G22)
NVLSLYNFKTTF

SEQ ID NO: 474 - protein, 12 aa, synthetic construct
  REGION 1..12: note = Synthetic: integrin; Laminin alpha-3 LG (MA3G27)
NAPFPKLSWTIQ

SEQ ID NO: 475 - protein, 12 aa, synthetic construct
  REGION 1..12: note = Synthetic: integrin; Laminin alpha-3 LG (MA3G28)
WTIQTTVDRGLL

SEQ ID NO: 476 - protein, 13 aa, synthetic construct
  REGION 1..13: note = Synthetic: integrin; Laminin alpha-3 LG (MA3G34)
DTINNGRDHMILI

SEQ ID NO: 477 - protein, 13 aa, synthetic construct
  REGION 1..13: note = Synthetic: integrin; Laminin alpha-3 LG (MA3G35)
MILISIGKSQKRM

SEQ ID NO: 478 - protein, 12 aa, synthetic construct
  REGION 1..12: note = Synthetic: integrin; Laminin alpha-3 LG (A3GXX)
PPFLMLLKGSTR

SEQ ID NO: 479 - protein, 13 aa, synthetic construct
  REGION 1..13: note = Synthetic: integrin; Laminin alpha-3 LG (MA3G57)
NQRLASFSNAQQS

SEQ ID NO: 480 - protein, 17 aa, synthetic construct
  REGION 1..17: note = Synthetic: integrin; Laminin alpha-3 LG (MA3G59)
ISNVFVQRMSQSPEVLD

SEQ ID NO: 481 - protein, 13 aa, synthetic construct
  REGION 1..13: note = Synthetic: integrin; Laminin alpha-3 LG (MA3G63)
KARSFNVNQLLQD

SEQ ID NO: 482 - protein, 12 aa, synthetic construct
  REGION 1..12: note = Synthetic: integrin; Laminin alpha-3 LG A3G75
KNSFMALYLSKG

SEQ ID NO: 483 - protein, 19 aa, synthetic construct
  REGION 1..19: note = Synthetic: integrin; Laminin alpha-3 LG A3G756
KNSFMALYLSKGRLVFALG

SEQ ID NO: 484 - protein, 19 aa, synthetic construct
  REGION 1..19: note = Synthetic: integrin; Laminin EF-3
RDSFVALYLSEGHVIFALG

SEQ ID NO: 485 - protein, 12 aa, synthetic construct
  REGION 1..12: note = Synthetic: integrin; Laminin alpha-3 LG MA3G70
KPRLQFSLDIQT

SEQ ID NO: 486 - protein, 12 aa, synthetic construct
  REGION 1..12: note = Synthetic: integrin; Laminin alpha-3 LG MA3G97
DGQWHSVTVSIK

SEQ ID NO: 487 - protein, 12 aa, synthetic construct
  REGION 1..12: note = Synthetic: integrin; Laminin alpha-4 LG (A4G4)
FVLYLGSKNAKK

SEQ ID NO: 488 - protein, 12 aa, synthetic construct
  REGION 1..12: note = Synthetic: integrin; Laminin alpha-4 LG (A4G6)
LAIKNDNLVYVY

SEQ ID NO: 489 - protein, 12 aa, synthetic construct
  REGION 1..12: note = Synthetic: integrin; Laminin alpha-4 LG (A4G10)
AYFSIVKIERVG

SEQ ID NO: 490 - protein, 12 aa, synthetic construct
  REGION 1..12: note = Synthetic: integrin; Laminin alpha-4 LG (A4G20)
DVISLYNFKHIY

SEQ ID NO: 491 - protein, 12 aa, synthetic construct
  REGION 1..12: note = Synthetic: integrin; Laminin alpha-4 LG (A4G24)
FFDGSSYAVVRD

SEQ ID NO: 492 - protein, 12 aa, synthetic construct
  REGION 1..12: note = Synthetic: integrin; Laminin alpha-4 LG (A4G31)
LHVFYDFGFSNG

SEQ ID NO: 493 - protein, 20 aa, synthetic construct
  REGION 1..20: note = Synthetic: integrin
LKKAQINDAKYREISIIYHN

SEQ ID NO: 494 - protein, 12 aa, synthetic construct
  REGION 1..12: note = Synthetic: integrin; Laminin alpha-4 LG (A4G47)
RAYFNGQSFIAS

SEQ ID NO: 495 - protein, 12 aa, synthetic construct
  REGION 1..12: note = Synthetic: integrin; Laminin alpha-4 LG (A4G59)
SRLRGKNPTKGK

SEQ ID NO: 496 - protein, 12 aa, synthetic construct
  REGION 1..12: note = Synthetic: integrin; Laminin alpha-4 LG (A4G69)
LHKKGKNSSKPK

SEQ ID NO: 497 - protein, 12 aa, synthetic construct
  REGION 1..12: note = Synthetic: integrin
RLKTRSSHGMIF

SEQ ID NO: 498 - protein, 12 aa, synthetic construct
  REGION 1..12: note = Synthetic: integrin; Laminin alpha-4 LG (A4G78)
GEKSQFSIRLKT

SEQ ID NO: 499 - protein, 12 aa, synthetic construct
  REGION 1..12: note = Synthetic: integrin; Laminin alpha-4 LG (A4G82)
TLFLAHGRLVFM

SEQ ID NO: 500 - protein, 12 aa, synthetic construct
  REGION 1..12: note = Synthetic: integrin; Laminin alpha-4 LG (A4G83)
LVFMFNVGHKKL

SEQ ID NO: 501 - protein, 20 aa, synthetic construct
  REGION 1..20: note = Synthetic: integrin; Laminin alpha-4 LG (A4G823)
TLFLAHGRLVFMFNVGHKKL

SEQ ID NO: 502 - protein, 19 aa, synthetic construct
  REGION 1..19: note = Synthetic: integrin; Laminin EF-4
DFMTLFLAHGRLVFMFNVG

SEQ ID NO: 503 - protein, 12 aa, synthetic construct
  REGION 1..12: note = Synthetic: integrin; Laminin alpha-4 LG (A4G84)
HKKLKIRSQEKY

SEQ ID NO: 504 - protein, 12 aa, synthetic construct
  REGION 1..12: note = Synthetic: integrin; Laminin alpha-4 LG (A4G90)
GAAWKIKGPIYL

SEQ ID NO: 505 - protein, 12 aa, synthetic construct
  REGION 1..12: note = Synthetic: integrin; Laminin alpha-4 LG (A4G107)
VIRDSNVVQLDV

SEQ ID NO: 506 - protein, 13 aa, synthetic construct
  REGION 1..13: note = Synthetic: alpha-5-beta-1; Laminin Laminin alpha-5 LN (S1)
EVNVTLDLGQVFH

SEQ ID NO: 507 - protein, 13 aa, synthetic construct
  REGION 1..13: note = Synthetic: alpha-4-beta-1, alpha-4-beta-7; Laminin Laminin alpha-5 LN (S2)
GQVFHVAYVLIKF

SEQ ID NO: 508 - protein, 15 aa, synthetic construct
  REGION 1..15: note = Synthetic: alpha-5-beta-1; Laminin Laminin alpha-5 LN (S6)
RDFTKATNIRLRFLR

SEQ ID NO: 509 - protein, 12 aa, synthetic construct
  REGION 1..12: note = Synthetic: alpha-5-beta-1; Laminin Laminin alpha-5 LN (S7)
NIRLRFLRTNTL

SEQ ID NO: 510 - protein, 12 aa, synthetic construct
  REGION 1..12: note = Synthetic: alpha-5-beta-1; Laminin alpha-5 LG1 (A5G3)
GKNTGDHFVLYM

SEQ ID NO: 511 - protein, 13 aa, synthetic construct
  REGION 1..13: note = Synthetic: integrin; Laminin alpha-5 LG1 (A5G19)
VVSLYNFEQTFML

SEQ ID NO: 512 - protein, 12 aa, synthetic construct
  REGION 1..12: note = Synthetic: integrin; Laminin alpha-5 LG2 (A5G26)
RFDQELRLVSYN

SEQ ID NO: 513 - protein, 12 aa, synthetic construct
  REGION 1..12: note = Synthetic: integrin; Laminin alpha-5 LG2 (A5G33)
ASKAIQVFLLGG

SEQ ID NO: 514 - protein, 12 aa, synthetic construct
  REGION 1..12: note = Synthetic: integrin; Laminin alpha-5 LG2 (A5G36)
TVFSVDQDNMLE

SEQ ID NO: 515 - protein, 12 aa, synthetic construct
  REGION 1..12: note = Synthetic: alpha-5-beta-1; Laminin alpha-5 LG3 (A5G63)
RLRGPQRVFDLH

SEQ ID NO: 516 - protein, 12 aa, synthetic construct
  REGION 1..12: note = Synthetic: integrin; Laminin alpha-5 LG3 (A5G66)
SRATAQKVSRRS

SEQ ID NO: 517 - protein, 12 aa, synthetic construct
  REGION 1..12: note = Synthetic: integrin; Laminin alpha-5 LG4 (A5G71)
GSLSSHLEFVGI

SEQ ID NO: 518 - protein, 12 aa, synthetic construct
  REGION 1..12: note = Synthetic: integrin; Laminin alpha-5 LG4 (A5G73)
RNRLHLSMLVRP

SEQ ID NO: 519 - protein, 13 aa, synthetic construct
  REGION 1..13: note = Synthetic: integrin; Laminin alpha-5 LG4 (A5G76)
APMSGRSPSLVLK

SEQ ID NO: 520 - protein, 12 aa, synthetic construct
  REGION 1..12: note = Synthetic: integrin; Laminin alpha-5 LG4 (A5G77)
LALFLSNGHFVA

SEQ ID NO: 521 - protein, 12 aa, synthetic construct
  REGION 1..12: note = Synthetic: integrin; Laminin alpha-5 LG4 (A5G81)
PGRWHKVSVRWE

SEQ ID NO: 522 - protein, 12 aa, synthetic construct
  REGION 1..12: note = Synthetic: integrin; Laminin alpha-5 LG4 (A5G82)
VRWGMQQIQLVV

SEQ ID NO: 523 - protein, 12 aa, synthetic construct
  REGION 1..12: note = Synthetic: integrin; Laminin alpha-5 LG5 (A5G94)
KMPYVSLELEMR

SEQ ID NO: 524 - protein, 12 aa, synthetic construct
  REGION 1..12: note = Synthetic: integrin; Laminin alpha-5 LG5 (A5G99)
VLLQANDGAGEF

SEQ ID NO: 525 - protein, 12 aa, synthetic construct
  REGION 1..12: note = Synthetic: integrin; Laminin alpha-5 LG5 (A5G101)
DGRWHRVAVIMG

SEQ ID NO: 526 - protein, 12 aa, synthetic construct
  REGION 1..12: note = Synthetic: integrin; Laminin alpha-5 LG5 (A5G109)
APVNVTASVQIQ

SEQ ID NO: 527 - protein, 12 aa, synthetic construct
  REGION 1..12: note = Synthetic: integrin; Laminin alpha-5 LG5 (A5G112)
KQGKALTQRHAK

SEQ ID NO: 528 - protein, 12 aa, synthetic construct
  REGION 1..12: note = Synthetic: integrin; Laminin Laminin vI (B-7)
AFGVLALWGTRV

SEQ ID NO: 529 - protein, 12 aa, synthetic construct
  REGION 1..12: note = Synthetic: integrin; Laminin Laminin vI (B-15)
IENVVTTFAPNR

SEQ ID NO: 530 - protein, 12 aa, synthetic construct
  REGION 1..12: note = Synthetic: integrin; Laminin Laminin vI (B-19)
LEAEFHFTHLIM

SEQ ID NO: 531 - protein, 12 aa, synthetic construct
  REGION 1..12: note = Synthetic: integrin; Laminin Laminin vI (B-20)
HLIMTFKTFRPA

SEQ ID NO: 532 - protein, 12 aa, synthetic construct
  REGION 1..12: note = Synthetic: integrin; Laminin Laminin vI (B-23)
KTWGVYRYFAYD

SEQ ID NO: 533 - protein, 12 aa, synthetic construct
  REGION 1..12: note = Synthetic: integrin; Laminin Laminin vI (B-31)
TNLRIKFVKLHT

SEQ ID NO: 534 - protein, 12 aa, synthetic construct
  REGION 1..12: note = Synthetic: integrin; Laminin Laminin vI (B-34)
REKYYYAVYDMV

SEQ ID NO: 535 - protein, 8 aa, synthetic construct
  REGION 1..8: note = Synthetic: integrin; Laminin Laminin v (B-54)
KRLVTGQR

SEQ ID NO: 536 - protein, 12 aa, synthetic construct
  REGION 1..12: note = Synthetic: integrin; I (B-187)
KDISEKVAVYST

SEQ ID NO: 537 - protein, 5 aa, synthetic construct
  REGION 1..5: note = Synthetic: integrin; Laminin III (B-96)
PDSGR

SEQ ID NO: 538 - protein, 5 aa, synthetic construct
  REGION 1..5: note = Synthetic: alpha-1-beta-1, alpha-3-beta-1; Laminin III (B-98)
YIGSR

SEQ ID NO: 539 - protein, 8 aa, synthetic construct
  REGION 1..8: note = Synthetic: alpha-1-beta-1, alpha-3-beta-1; Laminin III (B-98)
DPGYIGSR

SEQ ID NO: 540 - protein, 11 aa, synthetic construct
  REGION 1..11: note = Synthetic: integrin; Laminin III (B-116)
FALWDAIIGEL

SEQ ID NO: 541 - protein, 12 aa, synthetic construct
  REGION 1..12: note = Synthetic: integrin; Laminin II (B-123)
AAEPLKNIGILF

SEQ ID NO: 542 - protein, 12 aa, synthetic construct
  REGION 1..12: note = Synthetic: integrin; Laminin II (B-133)
DSITKYFQMSLE

SEQ ID NO: 543 - protein, 12 aa, synthetic construct
  REGION 1..12: note = Synthetic: integrin; Laminin I (B-160)
VILQQSAADIAR

SEQ ID NO: 544 - protein, 14 aa, synthetic construct
  REGION 1..14: note = Synthetic: integrin; Laminin Laminin Iv(B-77)
SPYTFIDSLVLMPY

SEQ ID NO: 545 - protein, 12 aa, synthetic construct
  REGION 1..12: note = Synthetic: integrin; Laminin I (B-187)
KDISEKVAVYST

SEQ ID NO: 546 - protein, 6 aa, synthetic construct
  REGION 1..6: note = Synthetic: integrin
LGTIPG

SEQ ID NO: 547 - protein, 12 aa, synthetic construct
  REGION 1..12: note = Synthetic: integrin; Laminin vI (C-3)
LWPLLAVLAAVA

SEQ ID NO: 548 - protein, 12 aa, synthetic construct
  REGION 1..12: note = Synthetic: alpha-v-beta-3, alpha-5-beta-1; Laminin vI (C-16)
KAFDITYVRLKF

SEQ ID NO: 549 - protein, 12 aa, synthetic construct
  REGION 1..12: note = Synthetic: integrin; Laminin vI (C-25)
AFSTLEGRPSAY

SEQ ID NO: 550 - protein, 13 aa, synthetic construct
  REGION 1..13: note = Synthetic: integrin; Laminin vI (C-28)
TDIRVTLNRLNTF

SEQ ID NO: 551 - protein, 13 aa, synthetic construct
  REGION 1..13: note = Synthetic: integrin; Laminin vI (C-30)
NEPKVLKSYYYAI

SEQ ID NO: 552 - protein, 12 aa, synthetic construct
  REGION 1..12: note = Synthetic: integrin; Laminin vI (C-31)
YYAISDFAVGGR

SEQ ID NO: 553 - protein, 13 aa, synthetic construct
  REGION 1..13: note = Synthetic: integrin; Laminin vI (C-35)
LPFFNDRPWRRAT

SEQ ID NO: 554 - protein, 14 aa, synthetic construct
  REGION 1..14: note = Synthetic: integrin; Laminin v (C-38)
FDPELYRSTGHGGH

SEQ ID NO: 555 - protein, 12 aa, synthetic construct
  REGION 1..12: note = Synthetic: integrin; Laminin v (C-50)
TNAVGYSVYDIS

SEQ ID NO: 556 - protein, 13 aa, synthetic construct
  REGION 1..13: note = Synthetic: integrin; Laminin Iv(C-57)
APVKFLGNQVLSY

SEQ ID NO: 557 - protein, 12 aa, synthetic construct
  REGION 1..12: note = Synthetic: integrin; Laminin Iv(C-59)
SFSFRVDRRDTR

SEQ ID NO: 558 - protein, 13 aa, synthetic construct
  REGION 1..13: note = Synthetic: integrin; Laminin Iv(C-64)
SETTVKYIFRLHE

SEQ ID NO: 559 - protein, 12 aa, synthetic construct
  REGION 1..12: note = Synthetic: integrin; Laminin Iv(C-67)
FQKLLNNLTSIK

SEQ ID NO: 560 - protein, 12 aa, synthetic construct
  REGION 1..12: note = Synthetic: integrin; Laminin Iv(C-68)
TSIKIRGTYSER

SEQ ID NO: 561 - protein, 6 aa, synthetic construct
  REGION 1..6: note = Synthetic: integrin; Laminin III (C75)
DPETGV

SEQ ID NO: 562 - protein, 12 aa, synthetic construct
  REGION 1..12: note = Synthetic: integrin; Laminin II (C-118)
TSAEAYNLLLRT

SEQ ID NO: 563 - protein, 12 aa, synthetic construct
  REGION 1..12: note = Synthetic: integrin; Laminin II (C102)
KEAEREVTDLLR

SEQ ID NO: 564 - protein, 12 aa, synthetic construct
  REGION 1..12: note = Synthetic: integrin; Laminin II (C-155)
SLLSQLNNLLDQ

SEQ ID NO: 565 - protein, 10 aa, synthetic construct
  REGION 1..10: note = Synthetic: integrin; Laminin
RNIAEIIKDI

SEQ ID NO: 566 - protein, 10 aa, synthetic construct
  REGION 1..10: note = Synthetic: integrin; Laminin
RDIAEIIKDI

SEQ ID NO: 567 - protein, 6 aa, synthetic construct
  REGION 1..6: note = Synthetic: integrin; Derived from Collagen alpha1(I)
GAPGER

SEQ ID NO: 568 - protein, 37 aa, synthetic construct
  REGION 1..37: note = Synthetic: Fibronectin; (FAB D3: 1-37) - highest affinity
FNKHTEIIEEDTNKDKPSYQFGGHNSVDFEEDTLPKV

SEQ ID NO: 569 - protein, 20 aa, synthetic construct
  REGION 1..20: note = Synthetic: Fibronectin; (FAB D3: 16-36) - high affinity
PSYQFGGHNSVDFEEDTLPK

SEQ ID NO: 570 - protein, 16 aa, synthetic construct
  REGION 1..16: note = Synthetic: Fibronectin; (FAB D3: 17-33) - medium affinity
SYQFGGHNSVDFEEDT

SEQ ID NO: 571 - protein, 17 aa, synthetic construct
  REGION 1..17: note = Synthetic: Fibronectin; (FAB D3: 20-36) - medium affinity
QFGGHNSVDFEEDTLPK

SEQ ID NO: 572 - protein, 16 aa, synthetic construct
  REGION 1..16: note = Synthetic: Fibronectin; (FAB D3: 21-36) -low affinity
FGGHNSVDFEEDTLPK

SEQ ID NO: 573 - protein, 109 aa, synthetic construct
  REGION 1..109: note = Synthetic: Fibronectin ; Fibronectin Type III(1)
NAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVS

SEQ ID NO: 574 - protein, 31 aa, synthetic construct
  REGION 1..31: note = Synthetic: Fibronectin ; Fibronectin Type III(1) fragment
NAPQPSHISKYILRWRPKNSVGRWKEATIPG

SEQ ID NO: 575 - protein, 31 aa, synthetic construct
  REGION 1..31: note = Synthetic: Fibronectin ; Fibronectin Type III(1) fragment
EATIPGHLNSYTIKGLKPGVVYEGQLISIQQ

SEQ ID NO: 576 - protein, 31 aa, synthetic construct
  REGION 1..31: note = Synthetic: Fibronectin ; Fibronectin Type III(1) fragment
LISIQQYGHQEVTRFDFTTTSTSTPVTSNTV

SEQ ID NO: 577 - protein, 34 aa, synthetic construct
  REGION 1..34: note = Synthetic: Fibronectin ; Fibronectin Type III(1) fragment
VTSNTVTGETTPFSPLVATSESVTEITASSFVVS

SEQ ID NO: 578 - protein, 44 aa, synthetic construct
  REGION 1..44: note = Synthetic: Fibronectin ; Fibronectin Type III(1) fragment (synthetic)
RWSHDNGVNYKIGEKWDRQGENGQMMSSTSLGNGKGEFKSDPHE

SEQ ID NO: 579 - protein, 42 aa, synthetic construct
  REGION 1..42: note = Synthetic: Fibronectin ; Fibronectin Type III(1) fragment (synthetic)
ATSYDDGKTYHVGEQWQKEYLGAISSSTSFGGQRGWRSDNSR

SEQ ID NO: 580 - protein, 19 aa, synthetic construct
  REGION 1..19: note = Synthetic: Fibronectin
DKPSYQFGGHNSVDFEEDT

SEQ ID NO: 581 - protein, 20 aa, synthetic construct
  REGION 1..20: note = Synthetic: Fibronectin
DKPSYQFGGHNSVDFEEDTL

SEQ ID NO: 582 - protein, 21 aa, synthetic construct
  REGION 1..21: note = Synthetic: Fibronectin
DKPSYQFGGHNSVDFEEDTLP

SEQ ID NO: 583 - protein, 22 aa, synthetic construct
  REGION 1..22: note = Synthetic: Fibronectin
DKPSYQFGGHNSVDFEEDTLPK

SEQ ID NO: 584 - protein, 18 aa, synthetic construct
  REGION 1..18: note = Synthetic: Fibronectin
KPSYQFGGHNSVDFEEDT

SEQ ID NO: 585 - protein, 19 aa, synthetic construct
  REGION 1..19: note = Synthetic: Fibronectin
KPSYQFGGHNSVDFEEDTL

SEQ ID NO: 586 - protein, 20 aa, synthetic construct
  REGION 1..20: note = Synthetic: Fibronectin
KPSYQFGGHNSVDFEEDTLP

SEQ ID NO: 587 - protein, 21 aa, synthetic construct
  REGION 1..21: note = Synthetic: Fibronectin
KPSYQFGGHNSVDFEEDTLPK

SEQ ID NO: 588 - protein, 17 aa, synthetic construct
  REGION 1..17: note = Synthetic: Fibronectin
PSYQFGGHNSVDFEEDT

SEQ ID NO: 589 - protein, 18 aa, synthetic construct
  REGION 1..18: note = Synthetic: Fibronectin
PSYQFGGHNSVDFEEDTL

SEQ ID NO: 590 - protein, 19 aa, synthetic construct
  REGION 1..19: note = Synthetic: Fibronectin
PSYQFGGHNSVDFEEDTLP

SEQ ID NO: 591 - protein, 20 aa, synthetic construct
  REGION 1..20: note = Synthetic: Fibronectin
PSYQFGGHNSVDFEEDTLPK

SEQ ID NO: 592 - protein, 20 aa, synthetic construct
  REGION 1..20: note = Synthetic: Heparin / syndecans; Derived from Heparin Binding Domans of Laminin
PPFLMLLKGSTRFNKTKTFR

SEQ ID NO: 593 - protein, 25 aa, synthetic construct
  REGION 1..25: note = Synthetic: Heparin /syndecans; Derived from Heparin Binding Domans of Laminin
RLVFALGTDGKKLRIKSKEKCNDGK

SEQ ID NO: 594 - protein, 27 aa, synthetic construct
  REGION 1..27: note = Synthetic: Heparin /syndecans; Derived from Heparin Binding Domans of Laminin
PLFLLHKKGKNLSKPKASQNKKGGKSK

SEQ ID NO: 595 - protein, 23 aa, synthetic construct
  REGION 1..23: note = Synthetic: Heparin /syndecans; Derived from Heparin Binding Domans of Laminin
TLFLAHGRLVYMFNVGHKKLKIR

SEQ ID NO: 596 - protein, 31 aa, synthetic construct
  REGION 1..31: note = Synthetic: Heparin /syndecans; Derived from Heparin Binding Domans of Laminin
TPGLGPRGLQATARKASRRSRQPARHPACML

SEQ ID NO: 597 - protein, 20 aa, synthetic construct
  REGION 1..20: note = Synthetic: Heparin /syndecans; Derived from Heparin Binding Domans of Laminin
RQRSRPGRWHKVSVRWEKNR

SEQ ID NO: 598 - protein, 12 aa, synthetic construct
  REGION 1..12: note = Synthetic: alpha-2-beta-1, Heparin; Derived from Collagen alpha1(Iv) HepII
LAGSCLARFSTM

SEQ ID NO: 599 - protein, 6 aa, synthetic construct
  REGION 1..6: note = Synthetic: Heparin; Derived from Collagen alpha1(I)
KGHRGF

SEQ ID NO: 600 - protein, 13 aa, synthetic construct
  REGION 1..13: note = Synthetic: Heparin; Derived from Collagen alpha1(I)
GDRGIKGHRGFSG

SEQ ID NO: 601 - protein, 15 aa, synthetic construct
  REGION 1..15: note = Synthetic: Heparin; Derived from Collagen alpha1(I)
GDLGRPGRKGRPGPP

SEQ ID NO: 602 - protein, 21 aa, synthetic construct
  REGION 1..21: note = Synthetic: Heparin; Derived from Collagen alpha1(I)
GHRGPTGRPGKRGKQGQKGDS

SEQ ID NO: 603 - protein, 6 aa, synthetic construct
  REGION 1..6: note = Synthetic: Heparin; Derived from Collagen alpha2(I)
KGIRGH

SEQ ID NO: 604 - protein, 13 aa, synthetic construct
  REGION 1..13: note = Synthetic: alpha-2-beta-1, Heparin; Derived from Collagen alpha1(Iv) HepIII
GEFYFDLRLKGDK

SEQ ID NO: 605 - protein, 11 aa, synthetic construct
  REGION 1..11: note = Synthetic: Heparin; Derived from Fibronectin III-1
KYILRWRPKNS

SEQ ID NO: 606 - protein, 16 aa, synthetic construct
  REGION 1..16: note = Synthetic: Heparin; Derived from Fibronectin III-13 (FN-C/H-III)
YRVRVTPKEKTGPMKE

SEQ ID NO: 607 - protein, 9 aa, synthetic construct
  REGION 1..9: note = Synthetic: alpha-5-beta-1, Heparin; Derived from FibronectinIII-13 (FN-C/H-Iv)
SPPRRARVT

SEQ ID NO: 608 - protein, 9 aa, synthetic construct
  REGION 1..9: note = Synthetic: Heparin; Derived from FibronectinIII-13
ATETTITIS

SEQ ID NO: 609 - protein, 32 aa, synthetic construct
  REGION 1..32: note = Synthetic: alpha-5-beta-1, Heparin; Derived from FibronectinIII-13
VSPPRRARVTDATETTITISWRTKTETITGFG

SEQ ID NO: 610 - protein, 11 aa, synthetic construct
  REGION 1..11: note = Synthetic: alpha-4-beta-1, Heparin; Derived from FibronectinIII-13
KPDVRSYTITG

SEQ ID NO: 611 - protein, 12 aa, synthetic construct
  REGION 1..12: note = Synthetic: alpha-4-beta-1, Heparin; Derived from FibronectinIII-13
ANGQTPIQRYIK

SEQ ID NO: 612 - protein, 19 aa, synthetic construct
  REGION 1..19: note = Synthetic: Heparin; Derived from FibronectinIII-14 (FN-C/H-I)
YEKPGSPPREVVPRPRPGV

SEQ ID NO: 613 - protein, 15 aa, synthetic construct
  REGION 1..15: note = Synthetic: Heparin; Derived from FibronectinIII-14 (FN-C/H-II)
KNNQKSEPLIGRKKT

SEQ ID NO: 614 - protein, 8 aa, synthetic construct
  REGION 1..8: note = Synthetic: integrin; Derived from Fibronectin IIICS-1
EILDVPST

SEQ ID NO: 615 - protein, 12 aa, synthetic construct
  REGION 1..12: note = Synthetic: alpha-2-beta-1, Heparin; Derived from Collagen alpha1(Iv) HepI
TAGSCLRKFSTM

SEQ ID NO: 616 - protein, 9 aa, synthetic construct
  REGION 1..9: note = Synthetic: Heparin; HPv
FRHRNRKGY

SEQ ID NO: 617 - protein, 16 aa, synthetic construct
  REGION 1..16: note = Synthetic: Heparin; HPv
KKQRFRHRNRKGYRSQ

SEQ ID NO: 618 - protein, 4 aa, synthetic construct
  REGION 1..4: note = Synthetic: Heparin; Bone sialoprotein
KRSR

SEQ ID NO: 619 - protein, 7 aa, synthetic construct
  REGION 1..7: note = Synthetic: Heparin, HSP; Bone sialoprotein
FHRRIKA

SEQ ID NO: 620 - protein, 12 aa, synthetic construct
  REGION 1..12: note = Synthetic: Heparin; LamininLaminin alpha-1 L4a (A51)
SINNTAVMQRLT

SEQ ID NO: 621 - protein, 12 aa, synthetic construct
  REGION 1..12: note = Synthetic: Heparin; Laminin alpha-1 L4a (A65)
ANVTHLLIRANY

SEQ ID NO: 622 - protein, 13 aa, synthetic construct
  REGION 1..13: note = Synthetic: integrin; Laminin alpha-1 L4a (A99)
AGTFALRGDNPQG

SEQ ID NO: 623 - protein, 13 aa, synthetic construct
  REGION 1..13: note = Synthetic: Heparin; Laminin alpha-5 LG2 (A5G27)
RLVSYSGVLFFLK

SEQ ID NO: 624 - protein, 6 aa, synthetic construct
  REGION 1..6: note = Synthetic: Heparin; Laminin alpha-5 LG2 (A5G)
GIIFFL

SEQ ID NO: 625 - protein, 12 aa, synthetic construct
  REGION 1..12: note = Synthetic: Heparin; Laminin alpha-5 LG2 (A5G35)
VLVRVERATVFS

SEQ ID NO: 626 - protein, 17 aa, synthetic construct
  REGION 1..17: note = Synthetic: Heparin; Laminin Laminin vI (B-30)
RIQNLLKITNLRIKFVK

SEQ ID NO: 627 - protein, 12 aa, synthetic construct
  REGION 1..12: note = Synthetic: Heparin; Laminin Iv(B-62)
GPGVVVVERQYI

SEQ ID NO: 628 - protein, 7 aa, synthetic construct
  REGION 1..7: note = Synthetic: Heparin; Laminin Iv(B-73)
RYVVLPR

SEQ ID NO: 629 - protein, 12 aa, synthetic construct
  REGION 1..12: note = Synthetic: SDC-4; Laminin alpha-1 L4a (A119)
LSNIDYILIKAS

SEQ ID NO: 630 - protein, 12 aa, synthetic construct
  REGION 1..12: note = Synthetic: SDC-4; Laminin alpha-1 L4a (A121)
LQQSRIANISME

SEQ ID NO: 631 - protein, 8 aa, synthetic construct
  REGION 1..8: note = Synthetic: SDC-1, -4; Laminin alpha-1 LG4 (AG73)
LQVQLSIR

SEQ ID NO: 632 - protein, 12 aa, synthetic construct
  REGION 1..12: note = Synthetic: SDC-1, -4; Laminin alpha-1 LG4 (AG73)
RKRLQVQLSIRT

SEQ ID NO: 633 - protein, 12 aa, synthetic construct
  REGION 1..12: note = Synthetic: SDC-1, -4; Laminin alpha-1 LG4 (AG75)
GLIYYVAHQNQM

SEQ ID NO: 634 - protein, 11 aa, synthetic construct
  REGION 1..11: note = Synthetic: SDC-4; Laminin alpha-5 LG3 (A5G64)
FDLHQNMGSVN

SEQ ID NO: 635 - protein, 12 aa, synthetic construct
  REGION 1..12: note = Synthetic: SDC-4; Laminin alpha-5 LG3 (A5G65)
QQNLGSVNVSTG

SEQ ID NO: 636 - protein, 8 aa, synthetic construct
  REGION 1..8: note = Synthetic: SDC-4; Derived from FibronectinIII-14 (FN-C/H-v)
WQPPRARI

SEQ ID NO: 637 - protein, 19 aa, synthetic construct
  REGION 1..19: note = Synthetic: SDC-4; Derived from FibronectinIII-14 (FN-C/H-v)
WQPPRARITGYIIKYEKPG

SEQ ID NO: 638 - protein, 13 aa, synthetic construct
  REGION 1..13: note = Synthetic: syndecan 2(w); Derived from Heparin Binding Domans of Laminin
KNSFMALYLSKGR

SEQ ID NO: 639 - protein, 15 aa, synthetic construct
  REGION 1..15: note = Synthetic: Heparan sulfate proteoglycans ; binds to HSGP with high affinity (DTx protein)
NGRKIRMRCRAIDGD

SEQ ID NO: 640 - protein, 15 aa, synthetic construct
  REGION 1..15: note = Synthetic: Heparan sulfate proteoglycans ; binds to HSGP with low affinity (DTx protein)
DVIRDKTKTKIESLK

SEQ ID NO: 641 - protein, 98 aa, synthetic construct
  REGION 1..98: note = Synthetic: hyaluronic acid; pH dependent (Link_TGS6)
GVYHREARSGKYKLTYAEAKAVCEFEGGHLATYKGLEAARKIGFHVCAAGWMAKGRVGYPIVKPGPPNCGFGKTGIIDYGIRLNRSERWDAYCYNPHA

SEQ ID NO: 642 - protein, 16 aa, synthetic construct
  REGION 1..16: note = Synthetic: Heparin; binds to heparin at low pH (high affinity)
KHAHLKKQVSDHIAVY

SEQ ID NO: 643 - protein, 12 aa, synthetic construct
  REGION 1..12: note = Synthetic: Heparin; binds to heparin at low pH (low affinity)
TTEPSEEHNHHK

SEQ ID NO: 644 - protein, 5 aa, synthetic construct
  REGION 1..5: note = Synthetic: Heparin; binds to heparin at low pH (lower affinity)
KHAHL

SEQ ID NO: 645 - protein, 12 aa, synthetic construct
  REGION 1..12: note = Synthetic: Heparin; binds to heparin at low pH (lower affinity)
TTEPSEEHNHHK

SEQ ID NO: 646 - protein, 16 aa, synthetic construct
  REGION 1..16: note = Synthetic: Heparin; binds to heparin at low pH (lower affinity)
TTEPSEEHNHHKHHDK

SEQ ID NO: 647 - protein, 6 aa, synthetic construct
  REGION 1..6: note = Synthetic: Heparin; binds to heparin at low pH (lower affinity)
HKGQHR

SEQ ID NO: 648 - protein, 22 aa, synthetic construct
  REGION 1..22: note = Synthetic: Heparin; binds to heparin at low pH
KVEHRVKKRPPTWRHNVRAKYT

SEQ ID NO: 649 - protein, 24 aa, synthetic construct
  REGION 1..24: note = Synthetic: Heparin; binds to heparin at low pH
GGKVEHRVKKRPPTWRHNVRAKYT

SEQ ID NO: 650 - protein, 11 aa, synthetic construct
  REGION 1..11: note = Synthetic: Heparin; binds to heparin at low pH
KKRPPTWRHNV

SEQ ID NO: 651 - protein, 6 aa, synthetic construct
  REGION 1..6: note = Synthetic: heparin; derived from thrombospondin
GTWSEW

SEQ ID NO: 652 - protein, 6 aa, synthetic construct
  REGION 1..6: note = Synthetic: heparin; derived from thrombospondin
GFWSEW

SEQ ID NO: 653 - protein, 6 aa, synthetic construct
  REGION 1..6: note = Synthetic: Fibronectin; derived from thrombospondin (highest affinity)
GGWSHW

SEQ ID NO: 654 - protein, 17 aa, synthetic construct
  REGION 1..17: note = Synthetic: Fibronectin; derived from thrombospondin (low affinity)
KRFKQDGGWSHWSPWSS

SEQ ID NO: 655 - protein, 14 aa, synthetic construct
  REGION 1..14: note = Synthetic: Fibronectin; derived from thrombospondin (medium affinity)
KRFKQDGGWSHWSP

SEQ ID NO: 656 - protein, 11 aa, synthetic construct
  REGION 1..11: note = Synthetic: Fibronectin; derived from thrombospondin (medium affinity)
GGWSHWSPWSS

SEQ ID NO: 657 - protein, 5 aa, synthetic construct
  REGION 1..5: note = Synthetic: Sulfated Glycoprotein; derived from thrombospondin (X= any amino acids)
  SITE 3: note = misc_feature - Xaa can be any naturally occurring amino acid
WSXWS

SEQ ID NO: 658 - protein, 4 aa, synthetic construct
  REGION 1..4: note = Synthetic: Sulfated Glycoprotein; derived from thrombospondin
WSHW

SEQ ID NO: 659-660 - skipped (2 entries)

SEQ ID NO: 661 - protein, 148 aa, synthetic construct
  REGION 1..148: note = Synthetic: heparin / heparan sulfate; Histidine Rich Glycoprotein (Histidine Rich Domain)
HPHKHHSHEQHPHGHHPHAHHPHEHDTHRQHPHGHHPHGHHPHGHHPHGHHPHGHHPHCHDFQDYGPCDPPPHNQGHCCHGHGPPPGHLRRRGPGKGPRPFHCRQIGSVYRLPPLRKGEVLPLPEANFPSFPLPHHKHPLKPDNQPFP

SEQ ID NO: 662 - protein, 35 aa, synthetic construct
  REGION 1..35: note = Synthetic: heparin / heparan sulfate; Histidine Rich Glycoprotein (Histidine Rich Domain)
DLHPHKHHSHEQHPHGHHPHAHHPHEHDTHRQHPH

SEQ ID NO: 663 - protein, 5 aa, synthetic construct
  REGION 1..5: note = Synthetic: heparin
GHHPH

SEQ ID NO: 664 - protein, 13 aa, synthetic construct
  REGION 1..13: note = Synthetic: heparin
VRIQRKKEKMKET

SEQ ID NO: 665 - protein, 9 aa, synthetic construct
  REGION 1..9: note = Synthetic: Collagen I
LHERHLNNN

SEQ ID NO: 666 - protein, 14 aa, synthetic construct
  REGION 1..14: note = Synthetic: MMP cleavable linker
GGGSGGGGPLGVRG

SEQ ID NO: 667 - protein, 14 aa, synthetic construct
  REGION 1..14: note = Synthetic: target- pH dependent heparin
GGGHHPHGPLGVRG

SEQ ID NO: 668 - protein, 14 aa, synthetic construct
  REGION 1..14: note = Synthetic: target- heparin
GVRIQRKKEKMKET

SEQ ID NO: 669 - protein, 14 aa, synthetic construct
  REGION 1..14: note = Synthetic: target- heparin
FHRRIKAGPLGVRG

SEQ ID NO: 670 - protein, 14 aa, synthetic construct
  REGION 1..14: note = Synthetic: MMP cleavable linker
GGGSGGGPAALIGG

SEQ ID NO: 671 - protein, 14 aa, synthetic construct
  REGION 1..14: note = Synthetic: target- pH dependent fibronectin
GGGWSHWGPLGVRG

SEQ ID NO: 672 - protein, 14 aa, synthetic construct
  REGION 1..14: note = Synthetic: target- Collagen Iv
KLWVLPKGPLGVRG

SEQ ID NO: 673 - protein, 14 aa, synthetic construct
  REGION 1..14: note = Synthetic: target- Collagen I
GGGSGLHERHLNNN

SEQ ID NO: 674-700 - skipped (27 entries)

SEQ ID NO: 701 - protein, 8 aa, synthetic construct
  REGION 1..8: note = Synthetic: MMP7
KRALGLPG

SEQ ID NO: 702 - protein, 40 aa, synthetic construct
  REGION 1..40: note = Synthetic: MMP7
DEDEDEDEDEDEDEDERPLALWRSDRDRDRDRDRDRDRDR

SEQ ID NO: 703 - skipped

SEQ ID NO: 704 - protein, 5 aa, synthetic construct
  REGION 1..5: note = Synthetic: MMP9
LEATA

SEQ ID NO: 705 - protein, 10 aa, synthetic construct
  REGION 1..10: note = Synthetic: MMP11
GGAANLVRGG

SEQ ID NO: 706 - protein, 10 aa, synthetic construct
  REGION 1..10: note = Synthetic: MMP14
SGRIGFLRTA

SEQ ID NO: 707 - protein, 6 aa, synthetic construct
  REGION 1..6: note = Synthetic: MMP
PLGLAG

SEQ ID NO: 708 - protein, 6 aa, synthetic construct
  REGION 1..6: note = Synthetic: MMP
  SITE 6: note = misc_feature - Xaa can be any naturally occurring amino acid
PLGLAX

SEQ ID NO: 709 - protein, 6 aa, synthetic construct
  REGION 1..6: note = Synthetic: MMP
  MOD_RES 4: note = methylcysteine
PLGCAG

SEQ ID NO: 710 - protein, 8 aa, synthetic construct
  REGION 1..8: note = Synthetic: MMP
ESPAYYTA

SEQ ID NO: 711 - protein, 6 aa, synthetic construct
  REGION 1..6: note = Synthetic: MMP
RLQLKL

SEQ ID NO: 712 - protein, 7 aa, synthetic construct
  REGION 1..7: note = Synthetic: MMP
RLQLKAC

SEQ ID NO: 713 - protein, 5 aa, synthetic construct
  REGION 1..5: note = Synthetic: MMP, MMP9, MMP14
  MOD_RES 2: note = citrulline
  MOD_RES 3: note = homophenylalanine
EPGYL

SEQ ID NO: 714 - protein, 5 aa, synthetic construct
  REGION 1..5: note = Synthetic: Urokinase plasminogen activator (uPA)
SGRSA

SEQ ID NO: 715 - protein, 4 aa, synthetic construct
  REGION 1..4: note = Synthetic: Urokinase plasminogen activator (uPA)
DAFK

SEQ ID NO: 716 - protein, 5 aa, synthetic construct
  REGION 1..5: note = Synthetic: Urokinase plasminogen activator (uPA)
GGGRR

SEQ ID NO: 717 - protein, 4 aa, synthetic construct
  REGION 1..4: note = Synthetic: Lysomal Enzyme
GFLG

SEQ ID NO: 718 - protein, 4 aa, synthetic construct
  REGION 1..4: note = Synthetic: Lysomal Enzyme
ALAL

SEQ ID NO: 719-720 - skipped (2 entries)

SEQ ID NO: 721 - protein, 5 aa, synthetic construct
  REGION 1..5: note = Synthetic: Cathepsin D
  MOD_RES 3: note = ethionine
PICFF

SEQ ID NO: 722 - protein, 8 aa, synthetic construct
  REGION 1..8: note = Synthetic: Cathepsin K
GGPRGLPG

SEQ ID NO: 723 - protein, 6 aa, synthetic construct
  REGION 1..6: note = Synthetic: Prostate Specific Antigen
HSSKLQ

SEQ ID NO: 724 - protein, 7 aa, synthetic construct
  REGION 1..7: note = Synthetic: Prostate Specific Antigen
HSSKLQL

SEQ ID NO: 725 - protein, 9 aa, synthetic construct
  REGION 1..9: note = Synthetic: Prostate Specific Antigen
HSSKLQEDA

SEQ ID NO: 726 - protein, 10 aa, synthetic construct
  REGION 1..10: note = Synthetic: Herpes Simplex virus Protease
LVLASSSFGY

SEQ ID NO: 727 - protein, 10 aa, synthetic construct
  REGION 1..10: note = Synthetic: HIv Protease
GVSQNYPIVG

SEQ ID NO: 728 - protein, 10 aa, synthetic construct
  REGION 1..10: note = Synthetic: CMv Protease
GVVQASCRLA

SEQ ID NO: 729 - skipped

SEQ ID NO: 730 - protein, 6 aa, synthetic construct
  REGION 1..6: note = Synthetic: Thrombin
DPRSFL

SEQ ID NO: 731 - protein, 6 aa, synthetic construct
  REGION 1..6: note = Synthetic: Thrombin
PPRSFL

SEQ ID NO: 732 - protein, 4 aa, synthetic construct
  REGION 1..4: note = Synthetic: Caspase-3
DEVD

SEQ ID NO: 733 - protein, 5 aa, synthetic construct
  REGION 1..5: note = Synthetic: Caspase-3
DEVDP

SEQ ID NO: 734 - protein, 8 aa, synthetic construct
  REGION 1..8: note = Synthetic: Caspase-3
KGSGDVEG

SEQ ID NO: 735 - protein, 6 aa, synthetic construct
  REGION 1..6: note = Synthetic: Interleukin 1-beta- converting enzyme
GWEHDG

SEQ ID NO: 736 - protein, 7 aa, synthetic construct
  REGION 1..7: note = Synthetic: Enterokinase
EDDDDKA

SEQ ID NO: 737 - protein, 9 aa, synthetic construct
  REGION 1..9: note = Synthetic: FAP
KQEQNPGST

SEQ ID NO: 738 - protein, 6 aa, synthetic construct
  REGION 1..6: note = Synthetic: Kallikrein 2
GKAFRR

SEQ ID NO: 739 - protein, 4 aa, synthetic construct
  REGION 1..4: note = Synthetic: Plasmin
DAFK

SEQ ID NO: 740 - protein, 4 aa, synthetic construct
  REGION 1..4: note = Synthetic: Plasmin
DVLK

SEQ ID NO: 741 - protein, 4 aa, synthetic construct
  REGION 1..4: note = Synthetic: Plasmin
DAFK

SEQ ID NO: 742 - protein, 7 aa, synthetic construct
  REGION 1..7: note = Synthetic: TOP
ALLLALL

SEQ ID NO: 743-746 - skipped (4 entries)

SEQ ID NO: 747 - protein, 248 aa, Homo sapiens
  REGION 1..248: note = misc_feature - scFv183
DIVMTQSPDSLAVSLGERATINCKSSQSVLYSNNNKNYLAWYQQKPGQPPKLLIYGASTRESWVPDRFSGSGSGTDFTLTISSLQAEDVAVYYCQQWYYYPYTFGQGTKVEIKGGGGSGGGGSGGGSGGGGSEVQLLESGGGLVQPGGSLRLSCAASGFTFSSYYMSWVRQAPGKGLEWVSDISGRGGQTNYADSVKGRFTISRDNSKNTLYLQMNSLRAEDTAVYYCARGGGSFANWGRGTLVTVSS

SEQ ID NO: 748 - protein, 113 aa, Homo sapiens
  REGION 1..113: note = misc_feature - vL region of scFv183
DIVMTQSPDSLAVSLGERATINCKSSQSVLYSNNNKNYLAWYQQKPGQPPKLLIYGASTRESWVPDRFSGSGSGTDFTLTISSLQAEDVAVYYCQQWYYYPYTFGQGTKVEIK

SEQ ID NO: 749 - protein, 116 aa, Homo sapiens
  REGION 1..116: note = misc_feature - vH region of scFv183
EVQLLESGGGLVQPGGSLRLSCAASGFTFSSYYMSWVRQAPGKGLEWVSDISGRGGQTNYADSVKGRFTISRDNSKNTLYLQMNSLRAEDTAVYYCARGGGSFANWGRGTLVTVSS

SEQ ID NO: 750 - protein, 17 aa, synthetic construct
  REGION 1..17: note = Synthetic: scFv183 vL HvR1
KSSQSVLYSNNNKNYLA

SEQ ID NO: 751 - protein, 7 aa, synthetic construct
  REGION 1..7: note = Synthetic: scFv183 vL HvR2
GASTRES

SEQ ID NO: 752 - protein, 9 aa, synthetic construct
  REGION 1..9: note = Synthetic: scFv183 vL HvR3
QQWYYYPYT

SEQ ID NO: 753 - protein, 6 aa, synthetic construct
  REGION 1..6: note = Synthetic: scFv183 vH HvR1
SSYYMS

SEQ ID NO: 754 - protein, 17 aa, synthetic construct
  REGION 1..17: note = Synthetic: scFv183 vH HvR2
DISGRGGQTNYADSVKG

SEQ ID NO: 755 - protein, 8 aa, synthetic construct
  REGION 1..8: note = Synthetic: scFv183 vH HvR3
RGGGSFAN

SEQ ID NO: 756 - protein, 226 aa, Homo sapiens
  REGION 1..226: note = misc_feature - IgG1 Fc (K360E/K409W) Knob
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 757 - protein, 226 aa, Homo sapiens
  REGION 1..226: note = misc_feature - h IgG1 Fc (Q347R/D399v/F405T) Hole
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 758-759 - skipped (2 entries)

SEQ ID NO: 760 - protein, 161 aa, Mus musculus
  REGION 1..161: note = misc_feature - m TGFb R II (1-161), wild-type ECD domain ofligand receptor
IPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPD

SEQ ID NO: 761 - protein, 137 aa, Homo sapiens
  REGION 1..137: note = misc_feature - hu TGFb R II (1-136), wild-type ECD domain ofligand receptor
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 762-772 - skipped (11 entries)

SEQ ID NO: 773 - protein, 121 aa, Homo sapiens
  REGION 1..121: note = misc_feature - Anti-TGF-beta-RII vH sequence
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSS

SEQ ID NO: 774 - protein, 107 aa, Homo sapiens
  REGION 1..107: note = misc_feature - Anti-TGF-beta-RII vL sequence
EIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIK

SEQ ID NO: 775 - protein, 243 aa, Homo sapiens
  REGION 1..243: note = misc_feature - Anti-TGF-beta-RII scFv sequence
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSGGGGSGGGGSGGGGSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIK

SEQ ID NO: 776 - protein, 243 aa, Homo sapiens
  REGION 1..243: note = misc_feature - Anti-TGF-beta-RII scFv sequence
EIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIKGGGGSGGGGSGGGGSQLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSS

SEQ ID NO: 777 - protein, 121 aa, Homo sapiens
  REGION 1..121: note = misc_feature - Anti-TGF-beta-RII vH sequence
QLQLQESGPGLVKPSETLSLTCTVSGGSISSSSYSWGWIRQPPGKGLEWIGSFYYSGITYYSPSLKSRIIISEDTSKNQFSLKLSSVTAADTAVYYCASGFTMIRGALDYWGQGTLVTVSS

SEQ ID NO: 778 - protein, 107 aa, Homo sapiens
  REGION 1..107: note = misc_feature - Anti-TGF-beta-RII vL sequence
EIVLTQSPATLSLSPGERATLSCRASQSVRSFLAWYQQKPGQAPRLLIYDASNRATGIPARFSGSGSGTDFTLTISSLEPEDFAVYYCQQRSNWPPTFGQGTKVEIK

SEQ ID NO: 779 - protein, 243 aa, Homo sapiens
  REGION 1..243: note = misc_feature - Anti-TGF-beta-RII scFv sequence
QLQLQESGPGLVKPSETLSLTCTVSGGSISSSSYSWGWIRQPPGKGLEWIGSFYYSGITYYSPSLKSRIIISEDTSKNQFSLKLSSVTAADTAVYYCASGFTMIRGALDYWGQGTLVTVSSGGGGSGGGGSGGGGSEIVLTQSPATLSLSPGERATLSCRASQSVRSFLAWYQQKPGQAPRLLIYDASNRATGIPARFSGSGSGTDFTLTISSLEPEDFAVYYCQQRSNWPPTFGQGTKVEIK

SEQ ID NO: 780 - protein, 243 aa, Homo sapiens
  REGION 1..243: note = misc_feature - Anti-TGF-beta-RII scFv sequence
EIVLTQSPATLSLSPGERATLSCRASQSVRSFLAWYQQKPGQAPRLLIYDASNRATGIPARFSGSGSGTDFTLTISSLEPEDFAVYYCQQRSNWPPTFGQGTKVEIKGGGGSGGGGSGGGGSQLQLQESGPGLVKPSETLSLTCTVSGGSISSSSYSWGWIRQPPGKGLEWIGSFYYSGITYYSPSLKSRIIISEDTSKNQFSLKLSSVTAADTAVYYCASGFTMIRGALDYWGQGTLVTVSS

SEQ ID NO: 781-799 - skipped (19 entries)

SEQ ID NO: 800 - protein, 613 aa, synthetic construct
  REGION 1..613: note = Synthetic: Construct B polypeptide sequence: m IL2-2x(SG4) MMPcs1 2x (G4S) IL2Ralpha mIgG1 Fc
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 801 - protein, 613 aa, synthetic construct
  REGION 1..613: note = Synthetic: Construct GGG
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFSQSIISTSPQSGGGGSGGGGGVRLGPGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 802 - protein, 611 aa, synthetic construct
  REGION 1..611: note = Synthetic: Construct AAA
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTSGGGGSGGGGGPLGVRGGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 803 - protein, 622 aa, synthetic construct
  REGION 1..622: note = Synthetic: Construct Y
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFSQSIISTSPQVRIQRKKEKMKETGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKIEGRMDGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 804 - protein, 613 aa, synthetic construct
  REGION 1..613: note = Synthetic: Construct AA
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGFHRRIKAGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 805 - protein, 613 aa, synthetic construct
  REGION 1..613: note = Synthetic: Construct BB
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGFHRRIKAGVRLGPGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 806 - protein, 613 aa, synthetic construct
  REGION 1..613: note = Synthetic: Construct CC
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGHHPHGHHPHGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 807 - protein, 613 aa, synthetic construct
  REGION 1..613: note = Synthetic: Construct DD
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGHHPHGHHPHGVRLGPGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 808 - protein, 612 aa, synthetic construct
  REGION 1..612: note = Synthetic: Construct EE
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGWSHWGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 809 - protein, 612 aa, synthetic construct
  REGION 1..612: note = Synthetic: Construct FF
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGWSHWGVRLGPGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 810 - protein, 613 aa, synthetic construct
  REGION 1..613: note = Synthetic: Construct GG
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGKLWVLPKGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 811 - protein, 613 aa, synthetic construct
  REGION 1..613: note = Synthetic: Construct HH
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGKLWVLPKGVRLGPGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 812 - protein, 613 aa, synthetic construct
  REGION 1..613: note = Synthetic: Construct II
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSLHERHLNNNGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 813 - protein, 613 aa, synthetic construct
  REGION 1..613: note = Synthetic: Construct JJ
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSLHERHLNNNGVRLGPGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 814 - protein, 616 aa, synthetic construct
  REGION 1..616: note = Synthetic: Construct KK
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQVRIQRKKEKMKETGVRLGPGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 815 - protein, 613 aa, synthetic construct
  REGION 1..613: note = Synthetic: Construct LL
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGPLGVRGFHRRIKAGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 816 - protein, 613 aa, synthetic construct
  REGION 1..613: note = Synthetic: Construct MM
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGVRLGPGFHRRIKAGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 817 - protein, 613 aa, synthetic construct
  REGION 1..613: note = Synthetic: Construct NN
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGPLGVRGGHHPHGHHPHELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 818 - protein, 613 aa, synthetic construct
  REGION 1..613: note = Synthetic: Construct OO
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGVRLGPGGHHPHGHHPHELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 819 - protein, 612 aa, synthetic construct
  REGION 1..612: note = Synthetic: Construct PP
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGPLGVRGGGWSHWGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 820 - protein, 612 aa, synthetic construct
  REGION 1..612: note = Synthetic: Construct QQ
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGVRLGPGGGWSHWGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 821 - protein, 613 aa, synthetic construct
  REGION 1..613: note = Synthetic: Construct RR
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGPLGVRGKLWVLPKGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 822 - protein, 613 aa, synthetic construct
  REGION 1..613: note = Synthetic: Construct SS
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGVRLGPGKLWVLPKGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 823 - protein, 613 aa, synthetic construct
  REGION 1..613: note = Synthetic: Construct TT
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGPLGVRGLHERHLNNNGELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 824 - protein, 613 aa, synthetic construct
  REGION 1..613: note = Synthetic: Construct UU
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGVRLGPGLHERHLNNNGELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 825 - protein, 613 aa, synthetic construct
  REGION 1..613: note = Synthetic: Construct vv
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGGHHPHGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 826 - protein, 613 aa, synthetic construct
  REGION 1..613: note = Synthetic: Construct WW
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGHHPHSGGGGGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 827 - protein, 613 aa, synthetic construct
  REGION 1..613: note = Synthetic: Construct XX
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGPLGVRGGHHPHGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 828 - protein, 623 aa, synthetic construct
  REGION 1..623: note = Synthetic: Construct YY
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGHHPHGHHPH

SEQ ID NO: 829 - protein, 618 aa, synthetic construct
  REGION 1..618: note = Synthetic: Construct ZZ
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGHHPH

SEQ ID NO: 830 - protein, 618 aa, synthetic construct
  REGION 1..618: note = Synthetic: Construct UUU
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGPLGVRGVRIQRKKEKMKETGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 831 - protein, 630 aa, synthetic construct
  REGION 1..630: note = Synthetic: Construct HHH
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGGSGVRIQRKKEKMKET

SEQ ID NO: 832 - protein, 618 aa, synthetic construct
  REGION 1..618: note = Synthetic: Construct III
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQVRIQRKKEKMKETGPLGVRGGGSKLWVLPKGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 833 - protein, 618 aa, synthetic construct
  REGION 1..618: note = Synthetic: Construct JJJ
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQKLWVLPKGGSGPLGVRGVRIQRKKEKMKETGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 834 - protein, 614 aa, synthetic construct
  REGION 1..614: note = Synthetic: Construct KKK
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQTLTYTWSGGGSGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 835 - protein, 624 aa, synthetic construct
  REGION 1..624: note = Synthetic: Construct LLL
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQVRIQRKKEKMKETGGSGPLGVRGVRIQRKKEKMKETGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 836 - protein, 620 aa, synthetic construct
  REGION 1..620: note = Synthetic: Construct MMM
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQLHERHLNNNGGSGPLGVRGVRIQRKKEKMKETGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 837 - protein, 611 aa, synthetic construct
  REGION 1..611: note = Synthetic: Construct CCC
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGHHPHGHHPHGVRLGPGGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 838 - protein, 611 aa, synthetic construct
  REGION 1..611: note = Synthetic: Construct DDD
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGHHPHGHHPHGPLGVRGGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 839 - protein, 614 aa, synthetic construct
  REGION 1..614: note = Synthetic: Construct EEE
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTVRIQRKKEKMKETGPLGVRGGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 840 - protein, 614 aa, synthetic construct; identical to SEQ ID NO: 839
  REGION 1..614: note = Synthetic: Construct FFF

SEQ ID NO: 841 - protein, 611 aa, synthetic construct
  REGION 1..611: note = Synthetic: Construct NNN
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTSGGKLWVLPKGPLGVRGGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 842 - protein, 616 aa, synthetic construct
  REGION 1..616: note = Synthetic: Construct OOO
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTVRIQRKKEKMKETGPLGVRGGGSKLWVLPKGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 843 - protein, 620 aa, synthetic construct
  REGION 1..620: note = Synthetic: Construct PPP
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQVRIQRKKEKMKETGGSGPLGVRGLHERHLNNNGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 844 - protein, 614 aa, synthetic construct
  REGION 1..614: note = Synthetic: Construct QQQ
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSLRELHLDNNGGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 845 - protein, 613 aa, synthetic construct
  REGION 1..613: note = Synthetic: Construct RRR
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGPLGVRGLRELHLDNNGELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 846 - protein, 620 aa, synthetic construct
  REGION 1..620: note = Synthetic: Construct SSS
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQLRELHLDNNGGSGPLGVRGVRIQRKKEKMKETGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 847 - protein, 620 aa, synthetic construct
  REGION 1..620: note = Synthetic: Construct TTT
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQVRIQRKKEKMKETGGSGPLGVRGLRELHLDNNGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 848 - protein, 217 aa, synthetic construct
  REGION 1..217: note = Synthetic: Construct FFFF
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSHHHHHH

SEQ ID NO: 849 - skipped

SEQ ID NO: 850 - protein, 123 aa, Camelus sp.
  REGION 1..123: note = misc_feature - 794B1P3B1, vHH
EVQLVESGGGLVQAGGSLRLSCAASERTFNMNVMGWFRQAPGKEREFVAAISWSTGGTSYGNFVKGRFTISGDNAKNTVYLEMNSLKPEDTAEYYCAAARFFTSLGAGEYAYRGQGTQVTVSS

SEQ ID NO: 851 - protein, 133 aa, Camelus sp.
  REGION 1..133: note = misc_feature - 794B1P3A3, vHH
QVQLVESGGGLVQAGDSLRLSCAPSGRTFGTYAPSRRTFGTYAMGWFRQAPGKEREFVADITWSGDRTYYADSVKGRFTISRDNPKSTVYLQMSSLKPEDTAVYYCAADSFMSKVLAGSAEYWGQGTQVTVSS

SEQ ID NO: 852 - protein, 114 aa, Camelus sp.
  REGION 1..114: note = misc_feature - 794B1P3C3, vHH
EVQLVESGGGLVQPGESLRLSCLASRTLSTFNVMAWYRQAPEKERELVAHVTNGTTLVADSVKGRFTISRDYTKNTVDLQMSKLKPEDTAVYYCRFWRGRYEYWGQGTQVTVSS

SEQ ID NO: 853 - protein, 122 aa, Camelus sp.
  REGION 1..122: note = misc_feature - 794B1P3A4, vHH
QVQLVESGGGLVQAGGSLRLSCAASVRTDSHNVVGWIRQAPGKEREFVAAISRSGYTSYTDSVKDRFTISRDNSRNTVYLQMNSLKPEDTALYYCAGRTFFSEFNVPPARNSGQGTQVTVSS

SEQ ID NO: 854 - protein, 133 aa, Camelus sp.
  REGION 1..133: note = misc_feature - 794B1P3B7, vHH
QVQLVESGGGLVQPGGSLRLSCAASGRTFGTYAPSRRTFGTYAMGWFRQAPGKEREFVADITWSGDRTYYADSVKGRFTISRDNPKSTVYLQMSSLKPEDTAVYYCAADSFMSKVLAGSAEYWGQGTQVTVSS

SEQ ID NO: 855 - protein, 122 aa, Camelus sp.
  REGION 1..122: note = misc_feature - 794B1P3C7, vHH
EVQLVESGGGLVQAGGSLRLSCAASGRALYLMGWFRQVPGKEREFVAGILWSSSRYADSVKGRFTISRDNAKNTVSLQMNSLKPEDTAVYYCAAAIRRGQDIATITSEYDYWGQGTQVTVSS

SEQ ID NO: 856 - protein, 122 aa, Camelus sp.
  REGION 1..122: note = misc_feature - 794B1P3G11, vHH
QVQLVESGGGLVQAGGSPRLSCAASGRTLYFMGWFRQVPGKEREFVAGILWSSTTYADSVKGRFTISRDNAKNTASLQMNSLKPEDTAVYYCAAAIRRGQDIPTMTSEYAYWGQGTQVTVSS

SEQ ID NO: 857 - protein, 226 aa, Homo sapiens
  REGION 1..226: note = misc_feature - h IgG1 Fc (L234A/L235A/P329G)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 858 - protein, 226 aa, Homo sapiens
  REGION 1..226: note = misc_feature - Human IgG1 Fc (D356K; D399K) charge variant 2
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 859 - protein, 226 aa, Homo sapiens
  REGION 1..226: note = misc_feature - h IgG1 Fc (L234A/L235A/P329G/K360E/K409W) Knob
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 860 - protein, 226 aa, Homo sapiens
  REGION 1..226: note = misc_feature - h IgG1 Fc (L234A/L235A/P329G/Q347R/D399v/F405T) Hole
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 861 - protein, 226 aa, Homo sapiens
  REGION 1..226: note = misc_feature - Human IgG1 (L234A/L235A/P329G/K392D/K409D) Fc charge variant 1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 862 - protein, 226 aa, Homo sapiens
  REGION 1..226: note = misc_feature - Human IgG1 Fc (L234A/L235A/P329G/D356K/ D399K) charge variant 2
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 863 - protein, 123 aa, Camelus sp.
  SITE 10: note = misc_feature - Xaa can be any naturally occurring amino acid
  REGION 1..123: note = misc_feature - 794B1P3E1, vHH
EVQLVESGGXLVQAGGSLRLSCAASERTFNMNVMGWFRQAPGKEREFVAAMSWSISGTSYGNSVKGRFTISGDNAKNTVYLEMNSLKPEDTAEYYCVAGRFFSSLGAGDYAYRGQGTQVTVSS

SEQ ID NO: 864 - protein, 123 aa, Camelus sp.
  REGION 1..123: note = misc_feature - 794B1P3G7, vHH
QVQLVESGGGLVQPGGSLRLSCAASGFTFADGVMAWVRQAPGKGHEWVSSISISVGSTSYADSVKGRFTISRDNAKNTLYLQMNSLKPEDTAVYYCAKARFFLQAGRLDFEYRGRGTQVTVSS

SEQ ID NO: 865 - protein, 133 aa, Camelus sp.
  REGION 1..133: note = misc_feature - 794B1P3G9, vHH
EVQLVESGGGLVQAGDSLRLSCAPSGRTFGTYAPSRRTFGTCAMGWFRPATGREGDFVSYINWSGDRTYYAHSVKGRFTISRDNPKRTEYLQMNNRAPEDTAVYYCAANTIMCKVVTGSAEYWEQGTQVTVSS

SEQ ID NO: 866 - protein, 123 aa, Camelus sp.
  REGION 1..123: note = misc_feature - 794B2P3G1, vHH
QVQLVESGGGLVQAGGSLRLSCAASGRTFSNYFAGWFRQPPGEEREFVASISWGGDRMFYTDAVKGRFTISRDNAKNTVDLQMNSLKPEDTAVYYCSADRFATALYNGNGNYWGQGTQVTVSS

SEQ ID NO: 867 - protein, 122 aa, Camelus sp.
  REGION 1..122: note = misc_feature - 794B2P3D2A, vHH
EVQLVESGGGLVQAGDSLRLSCAASGRTVSNYAMGWFRQAPGKGREWIVTSWTSGDARYEDSVKGRFTISRDHAKNTVYLQMNSLKPEDTGVYYCVADQFGSAILNGRAEYWGQGTQVTVSS

SEQ ID NO: 868 - protein, 120 aa, Camelus sp.
  REGION 1..120: note = misc_feature - 794B2P3D2B, vHH
EVQLVESGGGLVQAGGSLRLSCAASGRTFIGYTMGWFRQAPGKEREFVASIIWRGDRTRYADSVKGRFTISGDNAKNTVYLRMNSMKPEDTAVYYCAARSGSHFPSFDYWGQGTQVSVSS

SEQ ID NO: 869 - protein, 122 aa, Camelus sp.
  REGION 1..122: note = misc_feature - 794B2P3C10, vHH
EVQLVESGGGLVQPGGSLRLSCEVSGRILYIMGWFRQVPGKDREFVAGILWSSTKYGDFVNGRFTISRDNVKNTVSLQMNSLKPEDTAVYYCAAAIRRGQDIPTISSEYNYWGQGTQVTVSS

SEQ ID NO: 870 - protein, 119 aa, Camelus sp.
  REGION 1..119: note = misc_feature - 794B2P3F11, vHH
EVQLVESGGGLVQPGGSLRLSCAASGSISSMNVMGWYRQAPGKQREFVAGMNSRSVTSYDDSVQGRFTVSRDHTKNMVYLQMNSLKPEDTAIYYCAYSTWWSTLGNDVWGQGTQVTVSS

SEQ ID NO: 871-899 - skipped (29 entries)

SEQ ID NO: 900 - protein, 160 aa, Homo sapiens
  REGION 1..160: note = misc_feature - IL-10
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 901 - protein, 114 aa, Homo sapiens
  REGION 1..114: note = misc_feature - IL-15
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 902 - protein, 103 aa, Homo sapiens
  REGION 1..103: note = misc_feature - CXCL9
TPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 903 - protein, 77 aa, Homo sapiens
  REGION 1..77: note = misc_feature - CXCL10
VPLSRTVRCTCISISNQPVNPRSLEKLEIIPASQFCPRVEIIATMKKKGEKRCLNPESKAIKNLLKAVSKERSKRSP

SEQ ID NO: 904 - protein, 361 aa, Homo sapiens
  REGION 1..361: note = misc_feature - hu TGFb 1
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 905 - protein, 394 aa, Homo sapiens
  REGION 1..394: note = misc_feature - hu TGFb 2
LSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 906 - protein, 389 aa, Homo sapiens
  REGION 1..389: note = misc_feature - hu TGFb 3
LSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 907 - protein, 214 aa, synthetic construct
  REGION 1..214: note = Synthetic: Trastuzumab light chain, humanized
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 908 - protein, 450 aa, synthetic construct
  REGION 1..450: note = Synthetic: Trastuzumab heavy chain, humanized
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 909 - protein, 107 aa, synthetic construct [sequence omitted]

SEQ ID NO: 910 - protein, 120 aa, synthetic construct [sequence omitted]

SEQ ID NO: 911 - protein, 214 aa, synthetic construct
  REGION 1..214: note = Synthetic: Cetuximab light chain, chimeric (mouse/human)
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 912 - protein, 449 aa, synthetic construct
  REGION 1..449: note = Synthetic: Cetuximab heavy chain, chimeric (mouse/human)
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 913 - protein, 107 aa, synthetic construct
  REGION 1..107: note = Synthetic: Cetuximab vL, chimeric (mouse/human)
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 914 - protein, 118 aa, synthetic construct
  REGION 1..118: note = Synthetic: Cetuximab vH, chimeric (mouse/human)
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVS

SEQ ID NO: 915 - protein, 440 aa, Homo sapiens
  REGION 1..440: note = misc_feature - Nivolumab heavy chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 916 - protein, 214 aa, Homo sapiens
  REGION 1..214: note = misc_feature - Nivolumab light chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 917 - protein, 113 aa, Homo sapiens [sequence omitted]

SEQ ID NO: 918 - protein, 107 aa, Homo sapiens [sequence omitted]

SEQ ID NO: 919 - protein, 448 aa, synthetic construct
  REGION 1..448: note = Synthetic: atezolizumab heavy chain, humanized
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 920 - protein, 214 aa, synthetic construct
  REGION 1..214: note = Synthetic: atezolizumab light chain, humanized
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 921 - protein, 118 aa, synthetic construct [sequence omitted]

SEQ ID NO: 922 - protein, 107 aa, synthetic construct [sequence omitted]

SEQ ID NO: 923 - protein, 449 aa, synthetic construct
  REGION 1..449: note = Synthetic: Teplizumab heavy chain, humanized
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 924 - protein, 213 aa, synthetic construct
  REGION 1..213: note = Synthetic: Teplizumab light chain, humanized
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 925 - protein, 119 aa, synthetic construct [sequence omitted]

SEQ ID NO: 926 - protein, 106 aa, synthetic construct [sequence omitted]

SEQ ID NO: 927 - protein, 450 aa, Mus musculus
  REGION 1..450: note = misc_feature - muromonabheavy chain
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 928 - protein, 213 aa, Mus musculus
  REGION 1..213: note = misc_feature - muromonablight chain
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 929 - protein, 119 aa, Mus musculus [sequence omitted]

SEQ ID NO: 930 - protein, 106 aa, Mus musculus [sequence omitted]

SEQ ID NO: 931 - protein, 449 aa, synthetic construct
  REGION 1..449: note = Synthetic: otelixizumab heavy chain, chimeric (mouse/human)
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 932 - protein, 216 aa, synthetic construct
  REGION 1..216: note = Synthetic: otelixizumab light chain, chimeric (mouse/human)
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 933 - protein, 119 aa, synthetic construct [sequence omitted]

SEQ ID NO: 934 - protein, 111 aa, synthetic construct
  REGION 1..111: note = Synthetic: otelixizumab vL, chimeric (mouse/human)
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLR

SEQ ID NO: 935 - protein, 446 aa, synthetic construct
  REGION 1..446: note = Synthetic: visilizumab heavy chain, humanized
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPSK

SEQ ID NO: 936 - protein, 213 aa, synthetic construct
  REGION 1..213: note = Synthetic: visilizumab light chain, humanized
DIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 937 - protein, 120 aa, synthetic construct [sequence omitted]

SEQ ID NO: 938 - protein, 107 aa, synthetic construct
  REGION 1..107: note = Synthetic: visilizumab vL,humanized
DIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKR

SEQ ID NO: 939 - protein, 244 aa, Homo sapiens
  REGION 1..244: note = misc_feature - scFv (vL-vH)
DVVMTQSPLSLPVTLGQPASISCRSSQNIVHSNGNTYLEWYLQRPGQSPRLLIYKVSNRFSGVPDRFSGSGSGTDFTLKISRVEAEDVGVYYCFQGSHVPWTFGQGTKVEIKGGGGSGGGGSGGGGSEVQLVESGGGLVQPGGSLRLSCAASGFSFATYGVHWVRQSPGKGLEWLGVIWRGGSTDYSAAFMSRLTISKDNSKNTVYLQMNSLRAEDTAVYFCAKQAYGHYMDYWGQGTSVTVSS

SEQ ID NO: 940 - protein, 244 aa, Homo sapiens
  REGION 1..244: note = misc_feature - scFv (vH-vL)
EVQLVESGGGLVQPGGSLRLSCAASGFSFATYGVHWVRQSPGKGLEWLGVIWRGGSTDYSAAFMSRLTISKDNSKNTVYLQMNSLRAEDTAVYFCAKQAYGHYMDYWGQGTSVTVSSGGGGSGGGGSGGGGSDVVMTQSPLSLPVTLGQPASISCRSSQNIVHSNGNTYLEWYLQRPGQSPRLLIYKVSNRFSGVPDRFSGSGSGTDFTLKISRVEAEDVGVYYCFQGSHVPWTFGQGTKVEIK

SEQ ID NO: 941 - protein, 112 aa, Homo sapiens
  REGION 1..112: note = misc_feature - scFv vL region
DVVMTQSPLSLPVTLGQPASISCRSSQNIVHSNGNTYLEWYLQRPGQSPRLLIYKVSNRFSGVPDRFSGSGSGTDFTLKISRVEAEDVGVYYCFQGSHVPWTFGQGTKVEIK

SEQ ID NO: 942 - protein, 13 aa, Homo sapiens
  REGION 1..13: note = misc_feature - vL CDR1
SSQNIVHSNGNTY

SEQ ID NO: 943 - protein, 11 aa, Homo sapiens
  REGION 1..11: note = misc_feature - vL CDR2
KVSNRFSGVPD

SEQ ID NO: 944 - protein, 6 aa, Homo sapiens
  REGION 1..6: note = misc_feature - vL CDR3
GSHVPW

SEQ ID NO: 945 - protein, 117 aa, Homo sapiens
  REGION 1..117: note = misc_feature - scFv vH region
EVQLVESGGGLVQPGGSLRLSCAASGFSFATYGVHWVRQSPGKGLEWLGVIWRGGSTDYSAAFMSRLTISKDNSKNTVYLQMNSLRAEDTAVYFCAKQAYGHYMDYWGQGTSVTVSS

SEQ ID NO: 946 - protein, 10 aa, Homo sapiens
  REGION 1..10: note = misc_feature - vH CDR1
ASGFSFATYG

SEQ ID NO: 947 - protein, 16 aa, Homo sapiens
  REGION 1..16: note = misc_feature - vH CDR2
IWRGGSTDYSAAFMSR

SEQ ID NO: 948 - protein, 8 aa, Homo sapiens
  REGION 1..8: note = misc_feature - vH CDR3
QAYGHYMD

SEQ ID NO: 949 - protein, 448 aa, Homo sapiens
  REGION 1..448: note = misc_feature - anti-IL15 heavy chain
EVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 950 - protein, 118 aa, Homo sapiens
  REGION 1..118: note = misc_feature - anti-IL15 vH
EVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 951 - protein, 214 aa, Homo sapiens
  REGION 1..214: note = misc_feature - anti-IL15 light chain
EIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 952 - protein, 108 aa, Homo sapiens
  REGION 1..108: note = misc_feature - anti-IL15 vL
EIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISR

SEQ ID NO: 953 - protein, 250 aa, Homo sapiens
  REGION 1..250: note = misc_feature - ADL108-R3-09
EIVLTQSPGTLSLSPGERATLSCRASQSVSSNALAWYQQKPGQAPRLLIYGASSRATGIPDRFSGSGSGTDFTLTISRLEPEDFAVYYCQQAGSYPITFGQGTKVEIKGGGGSGGGGSGGGSGGGGSEVQLLESGGGLVQPGGSLRLSCAASGFTFTDYAMSWVRQAPGKGLEWVSGISGGGGSTRYADSVKGRFTISRDNSKNTLYLQMNSLRAEDTAVYYCARVVRGVISPYWYFDLWGRGTLVTVSS

SEQ ID NO: 954 - protein, 108 aa, Homo sapiens
  REGION 1..108: note = misc_feature - ADL108-R3-09 vL
EIVLTQSPGTLSLSPGERATLSCRASQSVSSNALAWYQQKPGQAPRLLIYGASSRATGIPDRFSGSGSGTDFTLTISRLEPEDFAVYYCQQAGSYPITFGQGTKVEIK

SEQ ID NO: 955 - protein, 123 aa, Homo sapiens
  REGION 1..123: note = misc_feature - ADL108-R3-09 vH
EVQLLESGGGLVQPGGSLRLSCAASGFTFTDYAMSWVRQAPGKGLEWVSGISGGGGSTRYADSVKGRFTISRDNSKNTLYLQMNSLRAEDTAVYYCARVVRGVISPYWYFDLWGRGTLVTVSS

SEQ ID NO: 956 - protein, 244 aa, Homo sapiens
  REGION 1..244: note = misc_feature - ADL108-R3-61
EIVLTQSPGTLSLSPGERATLSCRASQSVSSNYLAWYQQKPGQAPRLLIYGASSRATGIPDRFSGSGSGTDFTLTISRLEPEDFAVYYCQQYSSSPFTFGQGTKVEIKGGGGSGGGGSGGGSGGGGSEVQLLESGGGLVQPGGSLRLSCAASGFTFTDYWMSWVRQAPGKGLEWVSGIDGYGGGTNYADSVKGRFTISRDNSKNTLYLQMNSLRAEDTAVYYCAKARDSYADYWGQGTLVTVSS

SEQ ID NO: 957 - protein, 117 aa, Homo sapiens
  REGION 1..117: note = misc_feature - ADL108-R3-61 vH
EVQLLESGGGLVQPGGSLRLSCAASGFTFTDYWMSWVRQAPGKGLEWVSGIDGYGGGTNYADSVKGRFTISRDNSKNTLYLQMNSLRAEDTAVYYCAKARDSYADYWGQGTLVTVSS

SEQ ID NO: 958 - protein, 108 aa, Homo sapiens
  REGION 1..108: note = misc_feature - ADL108-R3-61 vL
EIVLTQSPGTLSLSPGERATLSCRASQSVSSNYLAWYQQKPGQAPRLLIYGASSRATGIPDRFSGSGSGTDFTLTISRLEPEDFAVYYCQQYSSSPFTFGQGTKVEIK

SEQ ID NO: 959 - protein, 251 aa, Homo sapiens
  REGION 1..251: note = misc_feature - ADL108-R3-79
DIVMTQSPDSLAVSLGERATINCKSSQSVLYSSNNENDLAWYQQKPGQPPKLLIYDASTRESGVPDRFSGSGSGTDFTLTISSLQAEDVAVYYCQQYSYRPLTFGQGTKVEIKGGGGSGGGGSGGGSGGGGSEVQLLESGGGLVQPGGSLRLSCAASGFTFSDTAMSWVRQAPGKGLEWVSAISGSGGSTYYADSVKGRFTISRDNSKNTLYLQMNSLRAEDTAVYYCANEWIPYGDYAFWGQGSLVTVSS

SEQ ID NO: 960 - protein, 119 aa, Homo sapiens
  REGION 1..119: note = misc_feature - ADL108-R3-79 vH
EVQLLESGGGLVQPGGSLRLSCAASGFTFSDTAMSWVRQAPGKGLEWVSAISGSGGSTYYADSVKGRFTISRDNSKNTLYLQMNSLRAEDTAVYYCANEWIPYGDYAFWGQGSLVTVSS

SEQ ID NO: 961 - protein, 113 aa, Homo sapiens
  REGION 1..113: note = misc_feature - ADL108-R3-79 vL
DIVMTQSPDSLAVSLGERATINCKSSQSVLYSSNNENDLAWYQQKPGQPPKLLIYDASTRESGVPDRFSGSGSGTDFTLTISSLQAEDVAVYYCQQYSYRPLTFGQGTKVEIK

SEQ ID NO: 962 - protein, 250 aa, Homo sapiens
  REGION 1..250: note = misc_feature - ADL108-R3-07
DIVMTQSPDSLAVSLGERATINCKSSQSVLYSSNNKNYLAWYQQKPGQPPKLLIYGASTRESGVPDRFSGSGSGTDFTLTISSLQAEDVAVYYCQQGYSAPFTFGQGTKVEIKGGGGSGGGGSGGGSGGGGSEVQLLESGGGLVQPGGSLRLSCAASGFTFTDTYMSWVRQAPGKGLEWVSAISGYGDTTKYADSVKGRFTISRDNSKNTLYLQMNSLRAEDTAVYYCARDRTASRFGYWGQGTLVTVSS

SEQ ID NO: 963 - protein, 118 aa, Homo sapiens
  REGION 1..118: note = misc_feature - ADL108-R3-07 vH
EVQLLESGGGLVQPGGSLRLSCAASGFTFTDTYMSWVRQAPGKGLEWVSAISGYGDTTKYADSVKGRFTISRDNSKNTLYLQMNSLRAEDTAVYYCARDRTASRFGYWGQGTLVTVSS

SEQ ID NO: 964 - protein, 113 aa, Homo sapiens
  REGION 1..113: note = misc_feature - ADL108-R3-07 vL
DIVMTQSPDSLAVSLGERATINCKSSQSVLYSSNNKNYLAWYQQKPGQPPKLLIYGASTRESGVPDRFSGSGSGTDFTLTISSLQAEDVAVYYCQQGYSAPFTFGQGTKVEIK

SEQ ID NO: 965 - protein, 246 aa, Homo sapiens
  REGION 1..246: note = misc_feature - ADL108-R3-10
DIVMTQSPDSLAVSLGERATINCKSSQSVLYSGNNENYLAWYQQKPGQPPKLLIYAASTRESGVPDRFSGSGSGTDFTLTISSLQAEDVAVYYCQQYQENPITFGQGTKVEIKGGGGSGGGGSGGGSGGGGSEVQLLESGGGLVQPGGSLRLSCAASGFTFSNYAMSWVRQAPGKGLEWVSGISGGGGSTDYADSVKGRFTISRDNSKNTLYLQMNSLRAEDTAVYYCARWPYGHWGQGTLVTVSS

SEQ ID NO: 966 - protein, 114 aa, Homo sapiens
  REGION 1..114: note = misc_feature - ADL108-R3-10 vH
EVQLLESGGGLVQPGGSLRLSCAASGFTFSNYAMSWVRQAPGKGLEWVSGISGGGGSTDYADSVKGRFTISRDNSKNTLYLQMNSLRAEDTAVYYCARWPYGHWGQGTLVTVSS

SEQ ID NO: 967 - protein, 113 aa, Homo sapiens
  REGION 1..113: note = misc_feature - ADL108-R3-10 vL
DIVMTQSPDSLAVSLGERATINCKSSQSVLYSGNNENYLAWYQQKPGQPPKLLIYAASTRESGVPDRFSGSGSGTDFTLTISSLQAEDVAVYYCQQYQENPITFGQGTKVEIK

SEQ ID NO: 968 - protein, 251 aa, Homo sapiens
  REGION 1..251: note = misc_feature - ADL108-R3-30
DIVMTQSPDSLAVSLGERATINCKSSQSVLDSYNNKNDLAWYQQKPGQPPKLLIYAASTRESGVPDRFSGSGSGTDFTLTISSLQAEDVAVYYCQQYYEAPYTFGQGTKVEIKGGGGSGGGGSGGGSGGGGSEVQLLESGGGLVQPGGSLRLSCAASGFTFSSYYMSWVRQAPGKGLEWVSEISGSGDSTYYADSVKGRFTISRDNSKNTLYLQMNSLRAEDTAVYYCASYYYYGSGFDYWGQGTLVTVSS

SEQ ID NO: 969 - protein, 113 aa, Homo sapiens
  REGION 1..113: note = misc_feature - ADL108-R3-30 vH
DIVMTQSPDSLAVSLGERATINCKSSQSVLDSYNNKNDLAWYQQKPGQPPKLLIYAASTRESGVPDRFSGSGSGTDFTLTISSLQAEDVAVYYCQQYYEAPYTFGQGTKVEIK

SEQ ID NO: 970 - protein, 119 aa, Homo sapiens
  REGION 1..119: note = misc_feature - ADL108-R3-30 vL
EVQLLESGGGLVQPGGSLRLSCAASGFTFSSYYMSWVRQAPGKGLEWVSEISGSGDSTYYADSVKGRFTISRDNSKNTLYLQMNSLRAEDTAVYYCASYYYYGSGFDYWGQGTLVTVSS

SEQ ID NO: 971 - protein, 255 aa, Homo sapiens
  REGION 1..255: note = misc_feature - ADL108-R3-38
DIVMTQSPDSLAVSLGERATINCKSSQSVLHSSNNENDLAWYQQKPGQPPKLLIYAASTRESGVPDRFSGSGSGTDFTLTISSLQAEDVAVYYCQQWYSEPYTFGQGTKVEIKGGGGSGGGGSGGGSGGGGSEVQLLESGGGLVQPGGSLRLSCAASGFTFSSTYMSWVRQAPGKGLEWVSGIYGGGTSYADSVKGRFTISRDNSKNTLYLQMNSLRAEDTAVYYCARENYYDILTGYYTQTETWGQGTLVTVSS

SEQ ID NO: 972 - protein, 113 aa, Homo sapiens
  REGION 1..113: note = misc_feature - ADL108-R3-38 vH
DIVMTQSPDSLAVSLGERATINCKSSQSVLHSSNNENDLAWYQQKPGQPPKLLIYAASTRESGVPDRFSGSGSGTDFTLTISSLQAEDVAVYYCQQWYSEPYTFGQGTKVEIK

SEQ ID NO: 973 - protein, 123 aa, Homo sapiens
  REGION 1..123: note = misc_feature - ADL108-R3-38 vL
EVQLLESGGGLVQPGGSLRLSCAASGFTFSSTYMSWVRQAPGKGLEWVSGIYGGGTSYADSVKGRFTISRDNSKNTLYLQMNSLRAEDTAVYYCARENYYDILTGYYTQTETWGQGTLVTVSS

SEQ ID NO: 974 - protein, 244 aa, Homo sapiens
  SITE 174: note = misc_feature - Xaa can be any naturally occurring amino acid
  REGION 1..244: note = misc_feature - ADL108-R3-43
EIVLTQSPGTLSLSPGERATLSCRASQSVSSNALAWYQQKPGQAPRLLIYGASSRATGIPDRFSGSGSGTDFTLTISRLEPEDFAVYYCQQYSEAPITFGQGTKVEIKGGGGSGGGGSGGGSGGGGSEVQLLESGGGLVQPGGSLRLSCAASGFTFTSYAMSWVRQAPGKGLEXVSGIDGYGGSTYYADSVKGRFTISRDNSKNTLYLQMNSLRAEDTAVYYCARAGIHLYDYWGQGTLVTVSS

SEQ ID NO: 975 - protein, 108 aa, Homo sapiens
  REGION 1..108: note = misc_feature - ADL108-R3-43 vH
EIVLTQSPGTLSLSPGERATLSCRASQSVSSNALAWYQQKPGQAPRLLIYGASSRATGIPDRFSGSGSGTDFTLTISRLEPEDFAVYYCQQYSEAPITFGQGTKVEIK

SEQ ID NO: 976 - protein, 117 aa, Homo sapiens
  SITE 47: note = misc_feature - Xaa can be any naturally occurring amino acid
  REGION 1..117: note = misc_feature - ADL108-R3-43 vL
EVQLLESGGGLVQPGGSLRLSCAASGFTFTSYAMSWVRQAPGKGLEXVSGIDGYGGSTYYADSVKGRFTISRDNSKNTLYLQMNSLRAEDTAVYYCARAGIHLYDYWGQGTLVTVSS

SEQ ID NO: 977 - protein, 248 aa, Homo sapiens
  REGION 1..248: note = misc_feature - ADL108-R3-53
EIVLTQSPGTLSLSPGERATLSCRASQSVSSSSLAWYQQKPGQAPRLLIYAASSRATGIPDRFSGSGSGTDFTLTISRLEPEDFAVYYCQQDSSSPFTFGQGTKVEIKGGGGSGGGGSGGGSGGGGSEVQLLESGGGLVQPGGSLRLSCAASGFTFSSYAMSWVRQAPGKGLEWVSAISGRGDYTKYADSVKGRFTISRDNSKNTLYLQMNSLRAEDTAVYYCARGTTIFGVTAFVYWGQGTLVTVSS

SEQ ID NO: 978 - protein, 121 aa, Homo sapiens
  REGION 1..121: note = misc_feature - ADL108-R3-53 vH
EVQLLESGGGLVQPGGSLRLSCAASGFTFSSYAMSWVRQAPGKGLEWVSAISGRGDYTKYADSVKGRFTISRDNSKNTLYLQMNSLRAEDTAVYYCARGTTIFGVTAFVYWGQGTLVTVSS

SEQ ID NO: 979 - protein, 108 aa, Homo sapiens
  REGION 1..108: note = misc_feature - ADL108-R3-53 vL
EIVLTQSPGTLSLSPGERATLSCRASQSVSSSSLAWYQQKPGQAPRLLIYAASSRATGIPDRFSGSGSGTDFTLTISRLEPEDFAVYYCQQDSSSPFTFGQGTKVEIK

SEQ ID NO: 980 - protein, 255 aa, Homo sapiens
  REGION 1..255: note = misc_feature - ADL108-R3-60
EIVLTQSPGTLSLSPGERATLSCRASQSVQSSALAWYQQKPGQAPRLLIYGASSRATGIPDRFSGSGSGTDFTLTISRLEPEDFAVYYCQQDGSWPLTFGQGTKVEIKGGGGSGGGGSGGGSGGGGSEVQLLESGGGLVQPGGSLRLSCAASGFTFSDYAMSWVRQAPGKGLEWVSRIDGGGGYTDYADSVKGRFTISRDNSKNTLYLQMNSLRAEDTAVYYCARHGSATIFGVVIHGYWYFDLWGRGTLVTVSS

SEQ ID NO: 981 - protein, 128 aa, Homo sapiens
  REGION 1..128: note = misc_feature - ADL108-R3-60 vH
EVQLLESGGGLVQPGGSLRLSCAASGFTFSDYAMSWVRQAPGKGLEWVSRIDGGGGYTDYADSVKGRFTISRDNSKNTLYLQMNSLRAEDTAVYYCARHGSATIFGVVIHGYWYFDLWGRGTLVTVSS

SEQ ID NO: 982 - protein, 108 aa, Homo sapiens
  REGION 1..108: note = misc_feature - ADL108-R3-60 vL
EIVLTQSPGTLSLSPGERATLSCRASQSVQSSALAWYQQKPGQAPRLLIYGASSRATGIPDRFSGSGSGTDFTLTISRLEPEDFAVYYCQQDGSWPLTFGQGTKVEIK

SEQ ID NO: 983 - protein, 253 aa, Homo sapiens
  REGION 1..253: note = misc_feature - ADL108-R3-87
DIVMTQSPDSLAVSLGERATINCKSSQSVLRSSNNKNNLAWYQQKPGQPPKLLIYWASTRESGVPDRFSGSGSGTDFTLTISSLQAEDVAVYYCQQSYYEPITFGQGTKVEIKGGGGSGGGGSGGGSGGGGSEVQLLESGGGLVQPGGSLRLSCAASGFTFSDTAMSWVRQAPGKGLEWVSGISGGGGYTNYADSVKGRFTISRDNSKNTLYLQMNSLRAEDTAVYYCAKSPDYDRRNYYDHWGQGTLVTVSS

SEQ ID NO: 984 - protein, 113 aa, Homo sapiens
  REGION 1..113: note = misc_feature - ADL108-R3-87 vL
DIVMTQSPDSLAVSLGERATINCKSSQSVLRSSNNKNNLAWYQQKPGQPPKLLIYWASTRESGVPDRFSGSGSGTDFTLTISSLQAEDVAVYYCQQSYYEPITFGQGTKVEIK

SEQ ID NO: 985 - protein, 121 aa, Homo sapiens
  REGION 1..121: note = misc_feature - ADL108-R3-87 vH
EVQLLESGGGLVQPGGSLRLSCAASGFTFSDTAMSWVRQAPGKGLEWVSGISGGGGYTNYADSVKGRFTISRDNSKNTLYLQMNSLRAEDTAVYYCAKSPDYDRRNYYDHWGQGTLVTVSS

SEQ ID NO: 986 - protein, 247 aa, Homo sapiens
  REGION 1..247: note = misc_feature - ADL108-R3-90
DIVMTQSPDSLAVSLGERATINCKSSQSVLYSGNNENYLAWYQQKPGQPPKLLIYDASTRESGVPDRFSGSGSGTDFTLTISSLQAEDVAVYYCQQWSNYPYTFGQGTKVEIKGGGGSGGGGSGGGSGGGGSEVQLLESGGGLVQPGGSLRLSCAASGFTFTDTYMSWVRQAPGKGLEWVSRIDGRGGGTYYADSVKGRFTISRDNSKNTLYLQMNSLRAEDTAVYYCAKGGSYGYWGQGTLVTVSS

SEQ ID NO: 987 - protein, 113 aa, Homo sapiens
  REGION 1..113: note = misc_feature - ADL108-R3-90 vL
DIVMTQSPDSLAVSLGERATINCKSSQSVLYSGNNENYLAWYQQKPGQPPKLLIYDASTRESGVPDRFSGSGSGTDFTLTISSLQAEDVAVYYCQQWSNYPYTFGQGTKVEIK

SEQ ID NO: 988 - protein, 115 aa, Homo sapiens
  REGION 1..115: note = misc_feature - ADL108-R3-90 vH
EVQLLESGGGLVQPGGSLRLSCAASGFTFTDTYMSWVRQAPGKGLEWVSRIDGRGGGTYYADSVKGRFTISRDNSKNTLYLQMNSLRAEDTAVYYCAKGGSYGYWGQGTLVTVSS

SEQ ID NO: 989 - protein, 250 aa, Homo sapiens
  REGION 1..250: note = misc_feature - anti-CXCL10 scFv vL-vH
GIQCEVKLVESGGGLVQPGGSLRLSCATSGFTFTDYYMSWVRQPPGKALEWLGFIRNKANGYTTEYSASVKGRFTISRDNQSILYLQMNTLRAEDSATYYCARDPTIGTVLCYGLLGSRNLSGGGGSGGGGSGGGGSEVQLQQSGPELEKPGASVKISCKASGYSFTGYNMNWVKQSNGKSLEWIGNIDPYYGGTSYNQKFKGKATLTVDKSSSTAYMQLKSLTSEDSAVYYCARSGTAWFAYWGQGTLV

SEQ ID NO: 990 - protein, 250 aa, Homo sapiens
  REGION 1..250: note = misc_feature - anti-CXCL10 scFv vH-vL
EVQLQQSGPELEKPGASVKISCKASGYSFTGYNMNWVKQSNGKSLEWIGNIDPYYGGTSYNQKFKGKATLTVDKSSSTAYMQLKSLTSEDSAVYYCARSGTAWFAYWGQGTLVGGGGSGGGGSGGGGSGIQCEVKLVESGGGLVQPGGSLRLSCATSGFTFTDYYMSWVRQPPGKALEWLGFIRNKANGYTTEYSASVKGRFTISRDNQSILYLQMNTLRAEDSATYYCARDPTIGTVLCYGLLGSRNLS

SEQ ID NO: 991 - protein, 113 aa, Homo sapiens
  REGION 1..113: note = misc_feature - anti-CXCL10 scFv vH
EVQLQQSGPELEKPGASVKISCKASGYSFTGYNMNWVKQSNGKSLEWIGNIDPYYGGTSYNQKFKGKATLTVDKSSSTAYMQLKSLTSEDSAVYYCARSGTAWFAYWGQGTLV

SEQ ID NO: 992 - protein, 122 aa, Homo sapiens
  REGION 1..122: note = misc_feature - anti-CXCL10 scFv vL
GIQCEVKLVESGGGLVQPGGSLRLSCATSGFTFTDYYMSWVRQPPGKALEWLGFIRNKANGYTTEYSASVKGRFTISRDNQSILYLQMNTLRAEDSATYYCARDPTIGTVLCYGLLGSRNLS

SEQ ID NO: 993 - protein, 5 aa, Homo sapiens
  REGION 1..5: note = misc_feature - vH CDR1
GYNMN

SEQ ID NO: 994 - protein, 16 aa, Homo sapiens
  REGION 1..16: note = misc_feature - vH CDR2
NIDPYYGGTSYNQKFK

SEQ ID NO: 995 - protein, 9 aa, Homo sapiens
  REGION 1..9: note = misc_feature - vH CDR3
SGTAWFAYW

SEQ ID NO: 996 - protein, 13 aa, Homo sapiens
  REGION 1..13: note = misc_feature - vL CDR1
ATSGFTFTDYYMS

SEQ ID NO: 997 - protein, 14 aa, Homo sapiens
  REGION 1..14: note = misc_feature - vL CDR2
IRNKANGYTTEYSA

SEQ ID NO: 998 - protein, 9 aa, Homo sapiens
  REGION 1..9: note = misc_feature - vL CDR3
ARDPTIGTV

SEQ ID NO: 999 - protein, 121 aa, Homo sapiens; identical to SEQ ID NO: 773
  REGION 1..121: note = misc_feature - vH region TGF1

SEQ ID NO: 1000 - protein, 107 aa, Homo sapiens; identical to SEQ ID NO: 774
  REGION 1..107: note = misc_feature - vL region TGF1

SEQ ID NO: 1001 - protein, 243 aa, Homo sapiens; identical to SEQ ID NO: 775
  REGION 1..243: note = misc_feature - scFv TGF1 (vH-vL)

SEQ ID NO: 1002 - protein, 243 aa, Homo sapiens; identical to SEQ ID NO: 776
  REGION 1..243: note = misc_feature - scFv TGF1 (vL-vH)

SEQ ID NO: 1003 - protein, 121 aa, Homo sapiens; identical to SEQ ID NO: 777
  REGION 1..121: note = misc_feature - vH region TGF3

SEQ ID NO: 1004 - protein, 107 aa, Homo sapiens; identical to SEQ ID NO: 778
  REGION 1..107: note = misc_feature - vL region TGF3

SEQ ID NO: 1005 - protein, 243 aa, Homo sapiens; identical to SEQ ID NO: 779
  REGION 1..243: note = misc_feature - scFv TGF3 (vH-vL)

SEQ ID NO: 1006 - protein, 243 aa, Homo sapiens; identical to SEQ ID NO: 780
  REGION 1..243: note = misc_feature - scFv TGF3 (vL-vH)

SEQ ID NO: 1007 - protein, 447 aa, Homo sapiens
  REGION 1..447: note = misc_feature - Fresolimumab heavy chain
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1008 - protein, 120 aa, Homo sapiens
  REGION 1..120: note = misc_feature - Fresolimumab vH
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSS

SEQ ID NO: 1009 - protein, 215 aa, Homo sapiens
  REGION 1..215: note = misc_feature - Fresolimumab light chain
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1010 - protein, 108 aa, Homo sapiens
  REGION 1..108: note = misc_feature - Fresolimumab vL
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 1011 - protein, 201 aa, Homo sapiens
  REGION 1..201: note = misc_feature - IL-10R beta
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPS

SEQ ID NO: 1012 - protein, 214 aa, Homo sapiens
  REGION 1..214: note = misc_feature - IL-10R alpha
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 1013 - protein, 175 aa, Homo sapiens
  REGION 1..175: note = misc_feature - IL-15R alpha (1-175)
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 1014 - protein, 170 aa, Homo sapiens
  REGION 1..170: note = misc_feature - IL-15R alpha (1-170)
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 1015 - protein, 77 aa, Homo sapiens
  REGION 1..77: note = misc_feature - IL-15R alpha (1-77)
ITCPPPMSVEHADIWVKSYSLYSRERYICNSGFKRKAGTSSLTECVLNKATNVAHWTTPSLKCIRDPALVHQRPAPP

SEQ ID NO: 1016 - protein, 214 aa, Homo sapiens; identical to SEQ ID NO: 16
  REGION 1..214: note = misc_feature - Human IL2Rbeta (1-214)

SEQ ID NO: 1017 - protein, 240 aa, Homo sapiens; identical to SEQ ID NO: 15
  REGION 1..240: note = misc_feature - Human IL2Rgamma (1-240)

SEQ ID NO: 1018 - protein, 162 aa, Homo sapiens
  REGION 1..162: note = misc_feature - Human IL2Rbeta (1-162)
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQ

SEQ ID NO: 1019 - protein, 120 aa, Homo sapiens
  REGION 1..120: note = misc_feature - Human IL2Rbeta (1-120)
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETH

SEQ ID NO: 1020 - protein, 368 aa, Homo sapiens
  REGION 1..368: note = misc_feature - CXCR3
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 1021 - protein, 21 aa, Homo sapiens
  REGION 1..21: note = misc_feature - CXCR3 (22-42)
NFSSSYDYGENESDSSSTSPP

SEQ ID NO: 1022 - protein, 161 aa, Mus musculus; identical to SEQ ID NO: 760
  REGION 1..161: note = misc_feature - m TGFb R II (1-161)

SEQ ID NO: 1023 - protein, 137 aa, Homo sapiens; identical to SEQ ID NO: 761
  REGION 1..137: note = misc_feature - hu TGFb R II (1-136)

SEQ ID NO: 1024 - protein, 463 aa, synthetic construct
  REGION 1..463: note = Synthetic: Construct vvv no TME control
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGPLGVRGGGGGSGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTHHHHHHG

SEQ ID NO: 1025 - protein, 473 aa, synthetic construct
  REGION 1..473: note = Synthetic: Construct WWW
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGPLGVRGGSVRIQRKKEKMKETGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTHHHHHHG

SEQ ID NO: 1026 - protein, 463 aa, synthetic construct
  REGION 1..463: note = Synthetic: Construct XXX
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGPLGVRGGGSKLWVLPKGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTHHHHHHG

SEQ ID NO: 1027 - protein, 696 aa, synthetic construct
  REGION 1..696: note = Synthetic: Construct YYY
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGPLGVRGVRIQRKKEKMKETGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1028 - protein, 693 aa, synthetic construct
  REGION 1..693: note = Synthetic: Construct ZZZ
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGPLGVRGGGSKLWVLPKGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1029 - protein, 693 aa, synthetic construct
  REGION 1..693: note = Synthetic: Construct AAAA
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGPLGVRGGLRELHLDNNGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1030 - protein, 696 aa, synthetic construct
  REGION 1..696: note = Synthetic: Construct BBBB
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGPLGVRGVRIQRKKEKMKETGGSKLWVLPKAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1031 - protein, 695 aa, synthetic construct
  REGION 1..695: note = Synthetic: Construct CCCC
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGPLGVRGGSKLWVLPKGGSKLWVLPKGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1032 - protein, 354 aa, synthetic construct
  REGION 1..354: note = Synthetic: Construct GGGG
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGVRIQRKKEKMKETGPLGVRGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 1033 - protein, 350 aa, synthetic construct
  REGION 1..350: note = Synthetic: Construct HHHH
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLWVLPKGGGPLGVRGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 1034 - protein, 716 aa, synthetic construct
  REGION 1..716: note = Synthetic: Construct IIII
HHHHHHGGSGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGKLWVLPKGSGPLGVRGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 1035 - protein, 718 aa, synthetic construct
  REGION 1..718: note = Synthetic: Construct JJJJ
HHHHHHGGSGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLVRIQRKKEKMKETGPLGVRGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 1036 - protein, 473 aa, synthetic construct
  REGION 1..473: note = Synthetic: Construct KKKK Arm 1; scFv Trastuzumab (vL-vH)-hu IgG1 Fc knob
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGASEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 1037 - protein, 616 aa, synthetic construct
  REGION 1..616: note = Synthetic: Construct KKKK Arm 2; Hu linker polypeptide-IL2(TME)- hu IgG1 Fc hole
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTSGGKLWVLPKGPLGVRGGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 1038 - protein, 473 aa, synthetic construct; identical to SEQ ID NO: 1036
  REGION 1..473: note = Synthetic: Construct LLLL Arm 1; scFv Trastuzumab (vL-vH)-hu IgG1 Fc knob

SEQ ID NO: 1039 - protein, 619 aa, synthetic construct
  REGION 1..619: note = Synthetic: Construct LLLL Arm 2; Hu linker polypeptide-IL2 (TME)- hu IgG1 Fc hole
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTVRIQRKKEKMKETGPLGVRGGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 1040 - protein, 472 aa, synthetic construct
  REGION 1..472: note = Synthetic: Construct MMMM Arm 1; scFv cetuximab (vL-vH)-hu IgG1 Fc knob
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGGGSGGGGSGGGASQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 1041 - protein, 616 aa, synthetic construct; identical to SEQ ID NO: 1037
  REGION 1..616: note = Synthetic: Construct MMMM Arm 2; Hu linker polypeptide-IL2(TME)- hu IgG1 Fc hole

SEQ ID NO: 1042 - protein, 625 aa, synthetic construct
  REGION 1..625: note = Synthetic: Construct NNNN; HuIL2(C125S)-vRIQRKKEKMKET-MMPcs1_(G4S)x2-hu IL2Ra(1-219; M25I)-GSGGGG- hu IgG1Fc- GGSGKLWvLPK
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTVRIQRKKEKMKETGPLGVRGGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGKLWVLPK

SEQ ID NO: 1043 - protein, 627 aa, synthetic construct
  REGION 1..627: note = Synthetic: Construct SSSS; Hu IL2(C125S)-vRIQRKKEKMKET -MMPcs1_(G4S)x2-hu IL2Ra(1-219; M25I)-GSGGGG- hu IgG1Fc - GGSGLRELHLDNN
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTVRIQRKKEKMKETGPLGVRGGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGLRELHLDNN

SEQ ID NO: 1044 - protein, 611 aa, synthetic construct
  REGION 1..611: note = Synthetic: Construct OOOO; Hu IL2(C125S)-2x(SG4)-MMPcs1- LRELHLDNN-hu IL2Ra(1-219; M25I)-GSGGGG- hu IgG1Fc
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTSGGGGSGGGGGPLGVRGLRELHLDNNGELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1045 - protein, 628 aa, synthetic construct
  REGION 1..628: note = Synthetic: Construct IIIII; huIL2(C125S)-SGGKLWvLPK-MMPcs1-2x(G4S)-hu IL2Ra(1-219; M25I)-GSGGGG-huIgG1-vRIQRKKEKMKET
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTSGGKLWVLPKGPLGVRGGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGVRIQRKKEKMKET

SEQ ID NO: 1046 - protein, 624 aa, synthetic construct
  REGION 1..624: note = Synthetic: Construct JJJJJ; huIL2(C125S)-SGGKLWvLPK-MMPcs1-2x(G4S)-hu IL2Ra(1-219; M25I)-GSGGGG-huIgG1-LRELHLDNN
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTSGGKLWVLPKGPLGVRGGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGLRELHLDNN

SEQ ID NO: 1047 - protein, 384 aa, synthetic construct
  REGION 1..384: note = Synthetic: Construct KKKKK; Fc knob MMPcs1-huIL2
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGSGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 1048 - protein, 623 aa, synthetic construct
  REGION 1..623: note = Synthetic: Construct LLLLL; h IL2 (C125S) vRIQRKKEKMKET- MMPcs1-3x(G4S)-hIL2Ra(M25I)-GSGGGG- hu IgG1 Fc knob
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTVRIQRKKEKMKETGPLGVRGGGGGSGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPG

SEQ ID NO: 1049 - protein, 620 aa, synthetic construct
  REGION 1..620: note = Synthetic: Construct MMMMM; h IL2 (C125S) SGGKLWvLPK- MMPcs1-3x(G4S)-hIL2Ra(M25I)-GSGGGG- hu IgG1 Fc knob
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTSGGKLWVLPKGPLGVRGGGGGSGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPG

SEQ ID NO: 1050 - protein, 617 aa, synthetic construct
  REGION 1..617: note = Synthetic: Construct NNNNN; huil2-vRIQRKKEKMKET-mmpcs1-il2Rb-Fc knob
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTVRIQRKKEKMKETGPLGVRGGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGSGGGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPG

SEQ ID NO: 1051 - protein, 614 aa, synthetic construct
  REGION 1..614: note = Synthetic: Construct OOOOO; huil2-KLWvLPK-mmpcs1-il2Rb-Fc knob
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTSGGKLWVLPKGPLGVRGGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGSGGGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPG

SEQ ID NO: 1052 - protein, 231 aa, synthetic construct
  REGION 1..231: note = Synthetic: Construct PPPPP; empty Fc hole
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 1053 - protein, 459 aa, synthetic construct
  REGION 1..459: note = Synthetic: Construct QQQQQ; huIL2Rb ECD- 3x(G4S)-Fc Hole
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 1054 - protein, 379 aa, synthetic construct
  REGION 1..379: note = Synthetic: Construct TTTTT; Fc hole -B2G1
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSQVQLVESGGGLVQAGGSLRLSCAASGRTFSNYFAGWFRQPPGEEREFVASISWGGDRMFYTDAVKGRFTISRDNAKNTVDLQMNSLKPEDTAVYYCSADRFATALYNGNGNYWGQGTQVTVSS

SEQ ID NO: 1055 - protein, 376 aa, synthetic construct
  REGION 1..376: note = Synthetic: Construct RRRRR; Fc hole -B2d2b
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQAGGSLRLSCAASGRTFIGYTMGWFRQAPGKEREFVASIIWRGDRTRYADSVKGRFTISGDNAKNTVYLRMNSMKPEDTAVYYCAARSGSHFPSFDYWGQGTQVSVSS

SEQ ID NO: 1056 - protein, 378 aa, synthetic construct
  REGION 1..378: note = Synthetic: Construct SSSSS; Fc hole -B2C10
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQPGGSLRLSCEVSGRILYIMGWFRQVPGKDREFVAGILWSSTKYGDFVNGRFTISRDNVKNTVSLQMNSLKPEDTAVYYCAAAIRRGQDIPTISSEYNYWGQGTQVTVSS

SEQ ID NO: 1057 - protein, 523 aa, synthetic construct
  REGION 1..523: note = Synthetic: Construct UUUUU; Fc hole- B2C10-5x(G4S)-B2D2b
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQPGGSLRLSCEVSGRILYIMGWFRQVPGKDREFVAGILWSSTKYGDFVNGRFTISRDNVKNTVSLQMNSLKPEDTAVYYCAAAIRRGQDIPTISSEYNYWGQGTQVTVSSGGGGSGGGGSGGGGSGGGGSGGGGSEVQLVESGGGLVQAGGSLRLSCAASGRTFIGYTMGWFRQAPGKEREFVASIIWRGDRTRYADSVKGRFTISGDNAKNTVYLRMNSMKPEDTAVYYCAARSGSHFPSFDYWGQGTQVSVSS

SEQ ID NO: 1058 - protein, 524 aa, synthetic construct
  REGION 1..524: note = Synthetic: Construct vvvvv; Fc hole- B2G1-5x(G4S)-B2D2b
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSQVQLVESGGGLVQAGGSLRLSCAASGRTFSNYFAGWFRQPPGEEREFVASISWGGDRMFYTDAVKGRFTISRDNAKNTVDLQMNSLKPEDTAVYYCSADRFATALYNGNGNYWGQGTQVTVSSGGGGSGGGGSGGGGSGGGGSGGGGSEVQLVESGGGLVQAGGSLRLSCAASGRTFIGYTMGWFRQAPGKEREFVASIIWRGDRTRYADSVKGRFTISGDNAKNTVYLRMNSMKPEDTAVYYCAARSGSHFPSFDYWGQGTQVSVSS

SEQ ID NO: 1059 - protein, 524 aa, synthetic construct
  REGION 1..524: note = Synthetic: Construct WWWWW; Fc hole- B2D2b-5x(G4S)-B2G1
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQAGGSLRLSCAASGRTFIGYTMGWFRQAPGKEREFVASIIWRGDRTRYADSVKGRFTISGDNAKNTVYLRMNSMKPEDTAVYYCAARSGSHFPSFDYWGQGTQVSVSSGGGGSGGGGSGGGGSGGGGSGGGGSQVQLVESGGGLVQAGGSLRLSCAASGRTFSNYFAGWFRQPPGEEREFVASISWGGDRMFYTDAVKGRFTISRDNAKNTVDLQMNSLKPEDTAVYYCSADRFATALYNGNGNYWGQGTQVTVSS

SEQ ID NO: 1060 - protein, 523 aa, synthetic construct
  REGION 1..523: note = Synthetic: Construct XXXXX; Fc hole- B2D2b-5x(G4S)-B2C10
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQAGGSLRLSCAASGRTFIGYTMGWFRQAPGKEREFVASIIWRGDRTRYADSVKGRFTISGDNAKNTVYLRMNSMKPEDTAVYYCAARSGSHFPSFDYWGQGTQVSVSSGGGGSGGGGSGGGGSGGGGSGGGGSEVQLVESGGGLVQPGGSLRLSCEVSGRILYIMGWFRQVPGKDREFVAGILWSSTKYGDFVNGRFTISRDNVKNTVSLQMNSLKPEDTAVYYCAAAIRRGQDIPTISSEYNYWGQGTQVTVSS

SEQ ID NO: 1061 - protein, 378 aa, synthetic construct
  REGION 1..378: note = Synthetic: Construct YYYYY; B2C10-5x(G4S)-Fc Hole
EVQLVESGGGLVQPGGSLRLSCEVSGRILYIMGWFRQVPGKDREFVAGILWSSTKYGDFVNGRFTISRDNVKNTVSLQMNSLKPEDTAVYYCAAAIRRGQDIPTISSEYNYWGQGTQVTVSSGGGGSGGGGSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 1062 - protein, 379 aa, synthetic construct
  REGION 1..379: note = Synthetic: Construct ZZZZZ; B2G1-5x(G4S)-Fc Hole
QVQLVESGGGLVQAGGSLRLSCAASGRTFSNYFAGWFRQPPGEEREFVASISWGGDRMFYTDAVKGRFTISRDNAKNTVDLQMNSLKPEDTAVYYCSADRFATALYNGNGNYWGQGTQVTVSSGGGGSGGGGSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 1063 - protein, 376 aa, synthetic construct; identical to SEQ ID NO: 1055
  REGION 1..376: note = Synthetic: Construct AAAAAA; B2D2b-5x(G4S)-Fc Hole

SEQ ID NO: 1064 - protein, 384 aa, synthetic construct
  REGION 1..384: note = Synthetic: Construct BBBBBB; Fc knob MMPcs1-huIL2 (C125S)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGSGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 1065 - protein, 619 aa, synthetic construct
  REGION 1..619: note = Synthetic: Construct CCCCCC; h IL2 (C125S) 2x(SG4)- MMPcs1- LRELHLDNN-1x(G4S)-hIL2Ra(M25I)-GSGGGG- hu IgG1 Fc knob
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTSGGGGSGGGGGPLGVRGLRELHLDNNGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPG

SEQ ID NO: 1066 - protein, 623 aa, synthetic construct
  REGION 1..623: note = Synthetic: Construct DDDDDD; h IL2 (C125S) vRIQRKKEKMKET- MMPcs1-3x(G4S)-hIL2Ra(M25I)-GSGGGG- hu IgG1 Fc
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTVRIQRKKEKMKETGPLGVRGGGGGSGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1067 - protein, 620 aa, synthetic construct
  REGION 1..620: note = Synthetic: Construct EEEEEE; h IL2 (C125S) SGGKLWvLPK- MMPcs1-3x(G4S)-hIL2Ra(M25I)-GSGGGG- hu IgG1 Fc
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTSGGKLWVLPKGPLGVRGGGGGSGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1068 - protein, 619 aa, synthetic construct
  REGION 1..619: note = Synthetic: Construct FFFFFF; h IL2 (C125S) 2x(SG4)- MMPcs1- LRELHLDNN-1x(G4S)-hIL2Ra(M25I)-GSGGGG- hu IgG1 Fc
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTSGGGGSGGGGGPLGVRGLRELHLDNNGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGSGGGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1069 - protein, 475 aa, synthetic construct
  REGION 1..475: note = Synthetic: Construct GGGGGG; Fc hole- 5x(G4S)- IL2Ra (1-219; M25I)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 1070 - protein, 465 aa, synthetic construct
  REGION 1..465: note = Synthetic: Construct HHHHHH; Fc hole- 3x(G4S)- IL2Ra(1-219; M25I)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 1071 - protein, 622 aa, synthetic construct
  REGION 1..622: note = Synthetic: Construct IIIIII; Fc hole- B2C10-5x(G4S)-IL2Ra(1-219; M25I)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQPGGSLRLSCEVSGRILYIMGWFRQVPGKDREFVAGILWSSTKYGDFVNGRFTISRDNVKNTVSLQMNSLKPEDTAVYYCAAAIRRGQDIPTISSEYNYWGQGTQVTVSSGGGGSGGGGSGGGGSGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 1072 - protein, 623 aa, synthetic construct
  REGION 1..623: note = Synthetic: Construct JJJJJJ; Fc hole- B2G1-5x(G4S)-IL2Ra(1-219; M25I)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSQVQLVESGGGLVQAGGSLRLSCAASGRTFSNYFAGWFRQPPGEEREFVASISWGGDRMFYTDAVKGRFTISRDNAKNTVDLQMNSLKPEDTAVYYCSADRFATALYNGNGNYWGQGTQVTVSSGGGGSGGGGSGGGGSGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 1073 - protein, 620 aa, synthetic construct
  REGION 1..620: note = Synthetic: Construct KKKKKK; Fc hole- B2D2b-5x(G4S)-IL2Ra(1-219; M25I)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQAGGSLRLSCAASGRTFIGYTMGWFRQAPGKEREFVASIIWRGDRTRYADSVKGRFTISGDNAKNTVYLRMNSMKPEDTAVYYCAARSGSHFPSFDYWGQGTQVSVSSGGGGSGGGGSGGGGSGGGGSGGGGSELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 1074 - protein, 517 aa, synthetic construct
  REGION 1..517: note = Synthetic: Construct LLLLLL; Fc hole- B2C10-5x(G4S)-B1C3
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQPGGSLRLSCEVSGRILYIMGWFRQVPGKDREFVAGILWSSTKYGDFVNGRFTISRDNVKNTVSLQMNSLKPEDTAVYYCAAAIRRGQDIPTISSEYNYWGQGTQVTVSSGGGGSGGGGSGGGGSGGGGSGGGGSEVQLVESGGGLVQPGESLRLSCLASRTLSTFNVMAWYRQAPEKERELVAHVTNGTTLVADSVKGRFTISRDYTKNTVDLQMSKLKPEDTAVYYCRFWRGRYEYWGQGTQVTVSS

SEQ ID NO: 1075 - protein, 518 aa, synthetic construct
  REGION 1..518: note = Synthetic: Construct MMMMMM; Fc hole- B2G1-5x(G4S)-B1C3
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSQVQLVESGGGLVQAGGSLRLSCAASGRTFSNYFAGWFRQPPGEEREFVASISWGGDRMFYTDAVKGRFTISRDNAKNTVDLQMNSLKPEDTAVYYCSADRFATALYNGNGNYWGQGTQVTVSSGGGGSGGGGSGGGGSGGGGSGGGGSEVQLVESGGGLVQPGESLRLSCLASRTLSTFNVMAWYRQAPEKERELVAHVTNGTTLVADSVKGRFTISRDYTKNTVDLQMSKLKPEDTAVYYCRFWRGRYEYWGQGTQVTVSS

SEQ ID NO: 1076 - protein, 524 aa, synthetic construct; identical to SEQ ID NO: 1059
  REGION 1..524: note = Synthetic: Construct NNNNNN; Fc hole- B2D2b-5x(G4S)-B1C3

SEQ ID NO: 1077 - protein, 465 aa, synthetic construct
  REGION 1..465: note = Synthetic: Construct OOOOOO; huIL2Ra(M25I; 1-219) - 3x(G4S)-Fc Hole
ELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 1078 - protein, 475 aa, synthetic construct
  REGION 1..475: note = Synthetic: Construct PPPPPP; huIL2Ra(M25I; 1-219) - 5x(G4S)-Fc Hole
ELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGGGGSGGGGSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 1079 - protein, 391 aa, synthetic construct
  REGION 1..391: note = Synthetic: Construct QQQQQQ; huIL2(C125S)- 3x(G4S)-Fc knob
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTSGGGGSGGGGGPLGVRGGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPG

SEQ ID NO: 1080 - protein, 397 aa, synthetic construct
  REGION 1..397: note = Synthetic: Construct RRRRRR; Fc knob - vRIQRKKEKMKET MMPcs1-huIL2 (C125S)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGSVRIQRKKEKMKETGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 1081 - protein, 391 aa, synthetic construct
  REGION 1..391: note = Synthetic: Construct SSSSSS; Fc knob- SGGKLWvLPK MMPcs1-huIL2 (C125S)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGSKLWVLPKGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 1082 - protein, 393 aa, synthetic construct
  REGION 1..393: note = Synthetic: Construct TTTTTT; Fc knob - LRELHLDNN MMPcs1-huIL2 (C125S)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGSLRELHLDNNGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 1083 - protein, 397 aa, synthetic construct
  REGION 1..397: note = Synthetic: Construct UUUUUU; huIL2 (C125S)- 2x(G4S)- MMPcs1-LRELHLDNN-1x(G4S)- Fc knob
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGGPLGVRGGGSLRELHLDNNGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPG

SEQ ID NO: 1084 - protein, 396 aa, synthetic construct
  REGION 1..396: note = Synthetic: Construct vvvvvv; Fc hole-B2G1-GSGG-vRIQRKKEKMKET
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSQVQLVESGGGLVQAGGSLRLSCAASGRTFSNYFAGWFRQPPGEEREFVASISWGGDRMFYTDAVKGRFTISRDNAKNTVDLQMNSLKPEDTAVYYCSADRFATALYNGNGNYWGQGTQVTVSSGSGGVRIQRKKEKMKET

SEQ ID NO: 1085 - protein, 392 aa, synthetic construct
  REGION 1..392: note = Synthetic: Construct WWWWWW; Fc hole-B2G1-GSGG- LRELHLDNN
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSQVQLVESGGGLVQAGGSLRLSCAASGRTFSNYFAGWFRQPPGEEREFVASISWGGDRMFYTDAVKGRFTISRDNAKNTVDLQMNSLKPEDTAVYYCSADRFATALYNGNGNYWGQGTQVTVSSGSGGLRELHLDNN

SEQ ID NO: 1086 - protein, 390 aa, synthetic construct
  REGION 1..390: note = Synthetic: Construct XXXXXX; Fc hole-B2G1-GSGG-KLWvLPK
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSQVQLVESGGGLVQAGGSLRLSCAASGRTFSNYFAGWFRQPPGEEREFVASISWGGDRMFYTDAVKGRFTISRDNAKNTVDLQMNSLKPEDTAVYYCSADRFATALYNGNGNYWGQGTQVTVSSGSGGKLWVLPK

SEQ ID NO: 1087 - protein, 395 aa, synthetic construct
  REGION 1..395: note = Synthetic: Construct YYYYYY; Fc hole-B2C10-GSGG-vRIQRKKEKMKET
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQPGGSLRLSCEVSGRILYIMGWFRQVPGKDREFVAGILWSSTKYGDFVNGRFTISRDNVKNTVSLQMNSLKPEDTAVYYCAAAIRRGQDIPTISSEYNYWGQGTQVTVSSGSGGVRIQRKKEKMKET

SEQ ID NO: 1088 - protein, 391 aa, synthetic construct
  REGION 1..391: note = Synthetic: Construct ZZZZZZ; Fc hole-B2C10-GSGG- LRELHLDNN
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQPGGSLRLSCEVSGRILYIMGWFRQVPGKDREFVAGILWSSTKYGDFVNGRFTISRDNVKNTVSLQMNSLKPEDTAVYYCAAAIRRGQDIPTISSEYNYWGQGTQVTVSSGSGGLRELHLDNN

SEQ ID NO: 1089 - protein, 389 aa, synthetic construct
  REGION 1..389: note = Synthetic: Construct AAAAAAA; Fc hole-B2C10-GSGG-KLWvLPK
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQPGGSLRLSCEVSGRILYIMGWFRQVPGKDREFVAGILWSSTKYGDFVNGRFTISRDNVKNTVSLQMNSLKPEDTAVYYCAAAIRRGQDIPTISSEYNYWGQGTQVTVSSGSGGKLWVLPK

SEQ ID NO: 1090 - protein, 393 aa, synthetic construct
  REGION 1..393: note = Synthetic: Construct BBBBBBB; Fc hole-B2D2b-GSGG-vRIQRKKEKMKET
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQAGGSLRLSCAASGRTFIGYTMGWFRQAPGKEREFVASIIWRGDRTRYADSVKGRFTISGDNAKNTVYLRMNSMKPEDTAVYYCAARSGSHFPSFDYWGQGTQVSVSSGSGGVRIQRKKEKMKET

SEQ ID NO: 1091 - protein, 390 aa, synthetic construct
  REGION 1..390: note = Synthetic: Construct CCCCCCC; Fc hole-B2D2b-GSGG- LRELHLDNN
SEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQAGGSLRLSCAASGRTFIGYTMGWFRQAPGKEREFVASIIWRGDRTRYADSVKGRFTISGDNAKNTVYLRMNSMKPEDTAVYYCAARSGSHFPSFDYWGQGTQVSVSSGSGGLRELHLDNN

SEQ ID NO: 1092 - protein, 387 aa, synthetic construct
  REGION 1..387: note = Synthetic: Construct DDDDDDD; Fc hole-B2D2b-GSGG-KLWvLPK
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQAGGSLRLSCAASGRTFIGYTMGWFRQAPGKEREFVASIIWRGDRTRYADSVKGRFTISGDNAKNTVYLRMNSMKPEDTAVYYCAARSGSHFPSFDYWGQGTQVSVSSGSGGKLWVLPK

SEQ ID NO: 1093 - protein, 548 aa, synthetic construct
  REGION 1..548: note = Synthetic: Construct EEEEEEE; Fc hole -B2D2b-2x(G4S)- MMPcs1-3x(G4S)-B2C10
MGWSCIILFLVATATGVHSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQAGGSLRLSCAASGRTFIGYTMGWFRQAPGKEREFVASIIWRGDRTRYADSVKGRFTISGDNAKNTVYLRMNSMKPEDTAVYYCAARSGSHFPSFDYWGQGTQVSVSSGGGGSGGGGSGPLGVRGGGGSGGGGSGGGGSEVQLVESGGGLVQPGGSLRLSCEVSGRILYIMGWFRQVPGKDREFVAGILWSSTKYGDFVNGRFTISRDNVKNTVSLQMNSLKPEDTAVYYCAAAIRRGQDIPTISSEYNYWGQGTQVTVSS

SEQ ID NO: 1094 - protein, 548 aa, synthetic construct
  REGION 1..548: note = Synthetic: Construct FFFFFFF; Fc hole -B2C10-2x(G4S)- MMPcs1-3x(G4S)- B2D2b
MGWSCIILFLVATATGVHSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQPGGSLRLSCEVSGRILYIMGWFRQVPGKDREFVAGILWSSTKYGDFVNGRFTISRDNVKNTVSLQMNSLKPEDTAVYYCAAAIRRGQDIPTISSEYNYWGQGTQVTVSSGGGGSGGGGSGPLGVRGGGGSGGGGSGGGGSEVQLVESGGGLVQAGGSLRLSCAASGRTFIGYTMGWFRQAPGKEREFVASIIWRGDRTRYADSVKGRFTISGDNAKNTVYLRMNSMKPEDTAVYYCAARSGSHFPSFDYWGQGTQVSVSS

SEQ ID NO: 1095 - protein, 549 aa, synthetic construct
  REGION 1..549: note = Synthetic: Construct GGGGGGG; Fc hole -B2G1-2x(G4S)- MMPcs1-3x(G4S)- B2D2b
MGWSCIILFLVATATGVHSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSQVQLVESGGGLVQAGGSLRLSCAASGRTFSNYFAGWFRQPPGEEREFVASISWGGDRMFYTDAVKGRFTISRDNAKNTVDLQMNSLKPEDTAVYYCSADRFATALYNGNGNYWGQGTQVTVSSGGGGSGGGGSGPLGVRGGGGSGGGGSGGGGSEVQLVESGGGLVQAGGSLRLSCAASGRTFIGYTMGWFRQAPGKEREFVASIIWRGDRTRYADSVKGRFTISGDNAKNTVYLRMNSMKPEDTAVYYCAARSGSHFPSFDYWGQGTQVSVSS

SEQ ID NO: 1096 - protein, 549 aa, synthetic construct
  REGION 1..549: note = Synthetic: Construct HHHHHHH; Fc hole -B2D2b-2x(G4S)- MMPcs1-3x(G4S)-B2G1
MGWSCIILFLVATATGVHSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSEVQLVESGGGLVQAGGSLRLSCAASGRTFIGYTMGWFRQAPGKEREFVASIIWRGDRTRYADSVKGRFTISGDNAKNTVYLRMNSMKPEDTAVYYCAARSGSHFPSFDYWGQGTQVSVSSGGGGSGGGGSGPLGVRGGGGSGGGGSGGGGSQVQLVESGGGLVQAGGSLRLSCAASGRTFSNYFAGWFRQPPGEEREFVASISWGGDRMFYTDAVKGRFTISRDNAKNTVDLQMNSLKPEDTAVYYCSADRFATALYNGNGNYWGQGTQVTVSS

SEQ ID NO: 1097 - protein, 426 aa, synthetic construct
  REGION 1..426: note = Synthetic: Construct IIIIIII; Fc knob MMPcs1-huIL2 (C125S)-vRIQRKKEKMKET
MGWSCIILFLVATATGVHSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGGSGGGGSGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGSGGVRIQRKKEKMKET

SEQ ID NO: 1098 - protein, 420 aa, synthetic construct
  REGION 1..420: note = Synthetic: Construct JJJJJJJ; Fc knob MMPcs1-huIL2 (C125S)- KLWvLPK
MGWSCIILFLVATATGVHSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGGSGGGGSGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGSGGKLWVLPK

SEQ ID NO: 1099 - protein, 422 aa, synthetic construct
  REGION 1..422: note = Synthetic: Construct KKKKKKK; Fc knob MMPcs1-huIL2 (C125S) - LRELHLDNN
MGWSCIILFLVATATGVHSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGGSGGGGSGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGSGGLRELHLDNN

SEQ ID NO: 1100 - protein, 400 aa, synthetic construct
  REGION 1..400: note = Synthetic: Construct LLLLLLL; Fc knob MMPcs1-huIL2 (C125S) - GWSHW
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGGSGGGGSGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGSGGGGWSHW

SEQ ID NO: 1101 - protein, 390 aa, synthetic construct
  REGION 1..390: note = Synthetic: Construct MMMMMMM; Fc knob MMPcs1-huIL2 (C125S)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGGSGGGGSGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 1102 - protein, 409 aa, synthetic construct
  REGION 1..409: note = Synthetic: Construct NNNNNNN; Fc knob MMPcs1-huIL2 (C125S)
MGWSCIILFLVATATGVHSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGGSGGGGSGPLGVRGGGGGSGGGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 1103 - protein, 420 aa, synthetic construct
  REGION 1..420: note = Synthetic: Construct OOOOOOO; Fc knob MMPcs1-huIL2 (C125S)-KLWvLPK
MGWSCIILFLVATATGVHSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGGSGGGGSGPLGVRGGGGGSGGGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGSGGKLWVLPK

SEQ ID NO: 1104 - protein, 422 aa, synthetic construct
  REGION 1..422: note = Synthetic: Construct PPPPPPP; Fc knob MMPcs1-huIL2 (C125S) - LRELHLDNN
MGWSCIILFLVATATGVHSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGGSGGGGSGPLGVRGGGGGSGGGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGSGGLRELHLDNN

SEQ ID NO: 1105 - protein, 421 aa, synthetic construct
  REGION 1..421: note = Synthetic: Construct QQQQQQQ; vRIQRKKEKMKET-Fc knob MMPcs1-huIL2 (C125S)
MGWSCIILFLVATATGVHSVRIQRKKEKMKETGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGSGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 1106 - protein, 417 aa, synthetic construct
  REGION 1..417: note = Synthetic: Construct RRRRRRR; LRELHLDNN -Fc knob MMPcs1-huIL2 (C125S)
MGWSCIILFLVATATGVHSLRELHLDNNGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGSGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 1107 - protein, 415 aa, synthetic construct
  REGION 1..415: note = Synthetic: Construct SSSSSSS; KLWvLPK-Fc knob MMPcs1-huIL2 (C125S)
MGWSCIILFLVATATGVHSKLWVLPKGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGSGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 1108 - protein, 414 aa, synthetic construct
  REGION 1..414: note = Synthetic: Construct TTTTTTT; fibronectin -Fc knob MMPcs1-huIL2 (C125S)
MGWSCIILFLVATATGVHSGGWSHWGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGSGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 1109 - protein, 384 aa, synthetic construct
  REGION 1..384: note = Synthetic: Construct UUUUUUU; Fc knob MMPscr-huIL2 (C125S)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGSGGGGGVRLGPGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 1110 - protein, 407 aa, synthetic construct
  REGION 1..407: note = Synthetic: Construct vvvvvvv; Fc knob MMPscr-huIL2 (C125S)- vRIQRKKEKMKET
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGGSGGGGSGGGGGVRLGPGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGSGGVRIQRKKEKMKET

SEQ ID NO: 1111 - protein, 401 aa, synthetic construct
  REGION 1..401: note = Synthetic: Construct WWWWWWW; Fc knob MMPscr-huIL2 (C125S)- KLWvLPK
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGGSGGGGSGGGGGVRLGPGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGSGGKLWVLPK

SEQ ID NO: 1112 - protein, 403 aa, synthetic construct
  REGION 1..403: note = Synthetic: Construct XXXXXXX; Fc knob MMPscr-huIL2 (C125S) - LRELHLDNN
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGGSGGGGSGGGGGVRLGPGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGSGGLRELHLDNN

SEQ ID NO: 1113 - protein, 400 aa, synthetic construct
  REGION 1..400: note = Synthetic: Construct YYYYYYY; Fc knob MMPscr-huIL2 (C125S) - GWSHW
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGGSGGGGSGGGGGVRLGPGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGSGGGGWSHW

SEQ ID NO: 1114 - protein, 379 aa, synthetic construct
  REGION 1..379: note = Synthetic: Construct ZZZZZZZ; Fc hole -B2G1
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSGGGGSQVQLVESGGGLVQAGGSLRLSCAASGRTFSNYFAGWFRQPPGEEREFVASISWGGDRMFYTDAVKGRFTISRDNAKNTVDLQMNSLKPEDTAVYYCSADRFATALYNGNGNYWGQGTQVTVSS

SEQ ID NO: 1115 - protein, 530 aa, synthetic construct
  REGION 1..530: note = Synthetic: Construct AAAAAAAA; Fc hole -B2G1-2x(G4S)- MMPcs1-3x(G4S)- B2D2b
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSGGGGSQVQLVESGGGLVQAGGSLRLSCAASGRTFSNYFAGWFRQPPGEEREFVASISWGGDRMFYTDAVKGRFTISRDNAKNTVDLQMNSLKPEDTAVYYCSADRFATALYNGNGNYWGQGTQVTVSSGGGGSGGGGSGPLGVRGGGGSGGGGSGGGGSEVQLVESGGGLVQAGGSLRLSCAASGRTFIGYTMGWFRQAPGKEREFVASIIWRGDRTRYADSVKGRFTISGDNAKNTVYLRMNSMKPEDTAVYYCAARSGSHFPSFDYWGQGTQVSVSS

SEQ ID NO: 1116 - protein, 384 aa, synthetic construct
  REGION 1..384: note = Synthetic: Construct BBBBBBBB; Fc knob MMPcs1-huIL2 (C125S)
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGGGGSGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 1117 - protein, 403 aa, synthetic construct
  REGION 1..403: note = Synthetic: Construct CCCCCCCC; Fc knob MMPcs1-huIL2 (C125S) - LRELHLDNN
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGGGGGSGGGGSGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGSGGLRELHLDNN

SEQ ID NO: 1118 - protein, 400 aa, synthetic construct
  REGION 1..400: note = Synthetic: Construct DDDDDDDD; Fc knob MMPcs1-huIL2 (C125S) - GWSHW
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGGGGGSGGGGSGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGSGGGGWSHW

SEQ ID NO: 1119 - protein, 407 aa, synthetic construct
  REGION 1..407: note = Synthetic: Construct EEEEEEEE; Fc knob MMPcs1-huIL2 (C125S)-vRIQRKKEKMKET
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGGGGGSGGGGSGGGGGPLGVRGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGSGGVRIQRKKEKMKET

SEQ ID NO: 1120 - protein, 693 aa, synthetic construct
  REGION 1..693: note = Synthetic: Construct TTTT; hu IL15Ra (1-77) linker hu IL15 (SG3)-GPLGvRG-4x(G4S)-IL2Rb (1-214)-(G4SG)-Hu IgG1 Fc
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGPLGVRGGGGGSGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1121 - protein, 693 aa, synthetic construct
  REGION 1..693: note = Synthetic: Construct QQQQ; hu IL15Ra (1-77) linker hu IL15 (SG3)-GPLGvRG-(G3S)- GGWSHW - 2x(G4S)-IL2Rb (1-214)-(G4SG)-Hu IgG1 Fc
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGPLGVRGGGGSGGWSHWGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1122 - protein, 712 aa, synthetic construct
  REGION 1..712: note = Synthetic: Construct UUUU; hu IL15Ra (1-77) linker hu IL15 (SG3)-MMPscr-(G3S)- GGWSHW - 2x(G4S)-IL2Rb (1-214)-(G4SG)-Hu IgG1 Fc
MGWSCIILFLVATATGVHSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGVRLGPGGGGSGGWSHWGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1123 - protein, 714 aa, synthetic construct
  REGION 1..714: note = Synthetic: Construct RRRR; hu IL15Ra (1-77) linker hu IL15 (SG3)-GPLGvRG-(G3S)- LRELHLDNN - 2x(G4S)-IL2Rb (1-214)-(G4SG)-Hu IgG1 Fc
MGWSCIILFLVATATGVHSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGPLGVRGGGSLRELHLDNNGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1124 - protein, 695 aa, synthetic construct
  REGION 1..695: note = Synthetic: Construct vvvv; hu IL15Ra (1-77) linker hu IL15 (SG3)-MMPscr-(G3S)- LRELHLDNN - 2x(G4S)-IL2Rb (1-214)-(G4SG)-Hu IgG1 Fc
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGVRLGPGGGSLRELHLDNNGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1125 - protein, 469 aa, synthetic construct
  REGION 1..469: note = Synthetic: Construct WWWW; Fc hole- 5x(G4S)- IL2Rb (1-213)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 1126 - protein, 459 aa, synthetic construct
  REGION 1..459: note = Synthetic: Construct XXXX; Fc hole- 3x(G4S)- IL2Rb (1-213)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 1127 - protein, 462 aa, synthetic construct
  REGION 1..462: note = Synthetic: Construct YYYY; Fc knob MMPcs1-hu IL15Ra (1-77) linker hu IL15
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGSGGGGGPLGVRGGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 1128 - protein, 463 aa, synthetic construct
  REGION 1..463: note = Synthetic: Construct ZZZZ; hu IL15Ra (1-77) linker hu IL15 (SG3)-GPLGvRG-2x(G4S)-hIgG1 Fc knob
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGPLGVRGGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPG

SEQ ID NO: 1129 - protein, 473 aa, synthetic construct
  REGION 1..473: note = Synthetic: Construct AAAAA; Fc knob-MMPcs1-hu IL15Ra (1-77) linker hu IL15 -KLWvLPK
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGSGGGGGPLGVRGGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGGKLWVLPK

SEQ ID NO: 1130 - protein, 473 aa, synthetic construct
  REGION 1..473: note = Synthetic: Construct BBBBB; Fc knob-MMPscr-hu IL15Ra (1-77) linker hu IL15-KLWvLPK
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGSGGGGGVRLGPGGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGGKLWVLPK

SEQ ID NO: 1131 - protein, 475 aa, synthetic construct
  REGION 1..475: note = Synthetic: Construct CCCCC; Fc knob-MMPcs1-hu IL15Ra (1-77) linker hu IL15-LRELHLDNN
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGSGGGGGPLGVRGGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGGLRELHLDNN

SEQ ID NO: 1132 - protein, 475 aa, synthetic construct
  REGION 1..475: note = Synthetic: Construct DDDDD; Fc knob-MMPscr-hu IL15Ra (1-77) linker hu IL15-LRELHLDNN
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPGGGGSGGGGGVRLGPGGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGGLRELHLDNN

SEQ ID NO: 1133 - protein, 472 aa, synthetic construct
  REGION 1..472: note = Synthetic: Construct EEEEE; hu IL15Ra (1-77) linker hu IL15 - KLWvLPK - MMPcs1-Fc knob
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGKLWVLPKGGGGPLGVRGGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPG

SEQ ID NO: 1134 - protein, 472 aa, synthetic construct
  REGION 1..472: note = Synthetic: Construct FFFFF; hu IL15Ra (1-77) linker hu IL15 - KLWvLPK - MMPscr-Fc knob
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGKLWVLPKGGGGVRLGPGGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPG

SEQ ID NO: 1135 - protein, 474 aa, synthetic construct
  REGION 1..474: note = Synthetic: Construct GGGGG; hu IL15Ra (1-77) linker hu IL15 - LRELHLDNN- MMPcs1-Fc knob
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGLRELHLDNNGGGGPLGVRGGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNRFTQKSLSLSPG

SEQ ID NO: 1136 - protein, 474 aa, synthetic construct; identical to SEQ ID NO: 1135
  REGION 1..474: note = Synthetic: Construct HHHHH; hu IL15Ra (1-77) linker hu IL15 -LRELHLDNN- MMPscr-Fc knob

SEQ ID NO: 1137 - protein, 693 aa, synthetic construct; identical to SEQ ID NO: 1120
  REGION 1..693: note = Synthetic: Construct PPPP; hu IL15Ra (1-77) linker hu IL15 (SG3)-GPLGvRG-4x(G4S)-IL2Rb (1-214)-(G4SG)-Hu IgG1 Fc

SEQ ID NO: 1138 - protein, 6 aa, synthetic construct
  REGION 1..6: note = Synthetic
GSGGGG

SEQ ID NO: 1139 - protein, 13 aa, synthetic construct
  REGION 1..13: note = Synthetic
VRIQRKKEKMKET

SEQ ID NO: 1140 - protein, 7 aa, synthetic construct
  REGION 1..7: note = Synthetic
FHRRIKA

SEQ ID NO: 1141 - protein, 5 aa, synthetic construct
  REGION 1..5: note = Synthetic
GHHPH

SEQ ID NO: 1142 - protein, 5 aa, synthetic construct
  REGION 1..5: note = Synthetic
GGGGS

SEQ ID NO: 1143 - protein, 5 aa, synthetic construct
  REGION 1..5: note = Synthetic
SGGGG

SEQ ID NO: 1144 - protein, 10 aa, synthetic construct
  REGION 1..10: note = Synthetic
FHRRIKAGGS

SEQ ID NO: 1145 - protein, 9 aa, synthetic construct
  REGION 1..9: note = Synthetic
GGWSHWGGS

SEQ ID NO: 1146 - protein, 10 aa, synthetic construct
  REGION 1..10: note = Synthetic
KLWVLPKGGS

SEQ ID NO: 1147 - protein, 10 aa, synthetic construct
  REGION 1..10: note = Synthetic
LHERHLNNNG

SEQ ID NO: 1148 - protein, 10 aa, synthetic construct
  REGION 1..10: note = Synthetic
SGGGGGHHPH

SEQ ID NO: 1149 - protein, 10 aa, synthetic construct
  REGION 1..10: note = Synthetic
GHHPHSGGGG

SEQ ID NO: 1150 - protein, 10 aa, synthetic construct
  REGION 1..10: note = Synthetic
GHHPHGGGGS

SEQ ID NO: 1151 - protein, 15 aa, synthetic construct
  REGION 1..15: note = Synthetic
VRIQRKKEKMKETGS

SEQ ID NO: 1152 - protein, 17 aa, synthetic construct
  REGION 1..17: note = Synthetic
GGSGVRIQRKKEKMKET

SEQ ID NO: 1153 - protein, 11 aa, synthetic construct
  REGION 1..11: note = Synthetic
TLTYTWSGGGS

SEQ ID NO: 1154 - protein, 10 aa, synthetic construct
  REGION 1..10: note = Synthetic
SGGKLWVLPK

SEQ ID NO: 1155 - protein, 12 aa, synthetic construct
  REGION 1..12: note = Synthetic
GGSKLWVLPKGS

SEQ ID NO: 1156 - protein, 9 aa, synthetic construct
  REGION 1..9: note = Synthetic
LRELHDNNG

SEQ ID NO: 1157 - protein, 10 aa, synthetic construct
  REGION 1..10: note = Synthetic
LRELHLDNNG

SEQ ID NO: 1158 - protein, 4 aa, synthetic construct
  REGION 1..4: note = Synthetic
SGGG

SEQ ID NO: 1159 - protein, 6 aa, synthetic construct
  REGION 1..6: note = Synthetic
HHHHHH

SEQ ID NO: 1160 - protein, 15 aa, synthetic construct
  REGION 1..15: note = Synthetic
GSVRIQRKKEKMKET

SEQ ID NO: 1161 - protein, 10 aa, synthetic construct
  REGION 1..10: note = Synthetic
GGSKLWVLPK

SEQ ID NO: 1162 - protein, 6 aa, synthetic construct
  REGION 1..6: note = Synthetic
GGGGSG

SEQ ID NO: 1163 - protein, 10 aa, synthetic construct
  REGION 1..10: note = Synthetic
GLRELHLDNN

SEQ ID NO: 1164 - protein, 11 aa, synthetic construct
  REGION 1..11: note = Synthetic
GGSGKLWVLPK

SEQ ID NO: 1165 - protein, 13 aa, synthetic construct
  REGION 1..13: note = Synthetic
GGSGLRELHLDNN

SEQ ID NO: 1166 - protein, 4 aa, synthetic construct
  REGION 1..4: note = Synthetic
GSGG

SEQ ID NO: 1167 - protein, 5 aa, synthetic construct
  REGION 1..5: note = Synthetic
GWSHW

SEQ ID NO: 1168 - protein, 4 aa, synthetic construct
  REGION 1..4: note = Synthetic
QLYV

(19) United States
(12) Patent Application Publication
(10) Pub. No.: US 2024/0417437 A1
(43) Pub. Date: Dec. 19, 2024

(54) Linker Polypeptides

(71) Applicant: Trutino Biosciences Inc., San Diego, CA (US)

(72) Inventors: Phillip S. Kim, San Diego, CA (US); Emma Langley, San Diego, CA (US); Hsieng Lu, Thousand Oaks, CA (US); Xinjun Liu, San Diego, CA (US); Chen Li, Irvine, CA (US)

(73) Assignee: Trutino Biosciences Inc., San Diego, CA (US)

(21) Appl. No.: 18/416,736

(22) Filed: Jan. 18, 2024

Related U.S. Application Data
(63) Continuation of application No. PCT/US22/73970, filed on Jul. 20, 2022.
(60) Provisional application No. 63/224,350, filed on Jul. 21, 2021.

(51) Int. Cl. C07K 14/55; A61K 35/00; A61K 38/00; C07K 14/54; C07K 14/71; C07K 14/715; C07K 16/24; C07K 16/28; C07K 16/32

(52) U.S. Cl. CPC C07K 14/55; A61K 35/00; C07K 14/5428; C07K 14/5443; C07K 14/71; C07K 14/7155; C07K 16/246; C07K 16/2809; C07K 16/2818; C07K 16/2827; C07K 16/2863; C07K 16/32; A61K 38/00; C07K 2317/31; C07K 2317/526; C07K 2319/30; C07K 2319/50

(57) ABSTRACT

This disclosure relates to linker polypeptides. In some embodiments, the linker polypeptide comprises a first targeting sequence; a second targeting sequence; and a first linker between the first targeting sequence and the second targeting sequence, the linker comprising a protease-cleavable polypeptide sequence. In some embodiments, the linker polypeptide comprises a first active domain; a second active domain; a pharmacokinetic modulator; and a first linker between the pharmacokinetic modulator and the first active domain, the first linker comprising a protease-cleavable polypeptide sequence. In some embodiments, the linker polypeptide comprises a first active domain; an inhibitory polypeptide sequence capable of blocking an activity of the first active domain; a first linker between the first active domain and the inhibitory polypeptide sequence, the linker comprising a protease-cleavable polypeptide sequence; and a first targeting sequence.

### CROSS-REFERENCE TO RELATED APPLICATIONS

This application claims the benefit of U.S. Provisional Patent Application No. 63/224,350, filed Jul. 21, 2021, which is incorporated herein by reference in its entirety for all purposes.

### INTRODUCTION AND SUMMARY

This disclosure relates to the field of linker polypeptides comprising one or more targeting sequences. The linker polypeptides are useful, e.g., for targeting to certain types of extracellular environments.

It can be beneficial to target protein therapeutics and other polypeptides to particular extracellular environments. It can also be beneficial to modulate the activity and/or pharmacokinetics to limit systemic and/or adverse effects.

For example, various forms of active domains, including but not limited to immunoglobulin antigen-binding domains, such as an Fv, scFv, Fab, or VHH, and cytokines and chemokines, such as IL-2, IL-10, IL-15, TGF-β, CXCL9, CXCL10, and others, play a significant role in targeting diseased cells and/or sustaining an effective immune cell response. In some cases, however, systemic administration of such compounds can activate immune cells throughout the body. Systemic activation can lead to systemic toxicity and indiscriminate activation of immune cells, including immune cells that respond to a variety of epitopes, antigens, and stimuli. The therapeutic potential of such therapy can be affected by these severe toxicities.

Peptide, immunoglobulin, and cytokine therapies can also suffer from a short serum half-life, sometimes on the order of several minutes. Thus, the high doses thereof that can be necessary to achieve an optimal effect can contribute to severe toxicities.

Further, in a traditional antibody, the immunoglobulin antigen-binding domains are fixed to a pharmacokinetic modulator, such as an Fc region. As such, the Fc region's activity is tied to the immunoglobulin antigen-binding domains' activities, and these regions and domains cannot operate independently, even when these activities are needed at different locations and/or at different times, or have differing requirements for Fc function, such as when one region or domain is for target destruction and another region or domain is for immunostimulation.

Accordingly, polypeptides that overcome the hurdles of systemic or untargeted function, severe toxicity, poor pharmacokinetics, and inseparable activities, are needed. Additionally, cancer cells may be stimulated by the presence of certain growth factors. Interfering with such stimulation while also increasing an immune response against the cancer cells would be beneficial. The present disclosure aims to meet one or more of these needs, provide other benefits, or at least provide the public with a useful choice.

In some aspects, linker polypeptides are provided, which can be targeted to certain types of extracellular environments through the use of targeting sequences. In some embodiments, the linker polypeptides can include a first targeting sequence; a second targeting sequence; and a first linker between the first targeting sequence and the second targeting sequence, the linker comprising a protease-cleavable polypeptide sequence. In some embodiments, the linker polypeptide can include a first active domain; a second active domain; a pharmacokinetic modulator; and a first linker between the pharmacokinetic modulator and the first active domain, or between the first active domain and the second active domain, the first linker comprising a protease-cleavable polypeptide sequence. In some embodiments, the linker polypeptide can include a first active domain; an inhibitory polypeptide sequence capable of blocking an activity of the first active domain; a first linker between the first active domain and the inhibitory polypeptide sequence, the linker comprising a protease-cleavable polypeptide sequence; and a first targeting sequence.

In some embodiments, different functions of different components of a linker polypeptide can be decoupled from each other and/or activated when one or more protease-cleavable polypeptide sequences are cleaved by one or more proteases. For example, cleaving a protease-cleavable polypeptide can allow an inhibitory polypeptide sequence to dissociate from a cytokine polypeptide sequence, and/or can allow an active domain (e.g., which may have an immunostimulatory function) to disassociate from the remainder of the linker polypeptide (e.g., which may have a target-destroying function).

Many tumors and tumor microenvironments exhibit aberrant expression and activation of proteases. The present disclosure provides linker polypeptides with components that may be decoupled from each other and/or activated through proteolytic cleavage, such that they become active when they come in contact with proteases in a tumor or tumor microenvironment. In some cases, for example, this can lead to an increase in active domains (e.g., cytokines or immunoglobulin domains) in and around the tumor or tumor microenvironment relative to the rest of a subject's body or healthy tissue. One exemplary advantage that can result is the formation of gradients of the active domain. Such a gradient can form when a linker polypeptide is administered and selectively or preferentially becomes activated in the tumor or tumor microenvironment and subsequently diffuses out of these areas to the rest of the body. These gradients can, e.g., increase the trafficking of immune cells to the tumor and tumor microenvironment. Immune cells that traffic to the tumor can infiltrate the tumor. Infiltrating immune cells can mount an immune response against the cancer. Infiltrating immune cells can also secrete their own chemokines and cytokines. The cytokines can have either or both of autocrine and paracrine effects within the tumor and tumor microenvironment. In some cases, the immune cells include T cells, such as T effector cells or cytotoxic T cells, or NK cells.

Also described herein are methods of treatment and methods of administrating the linker polypeptides described herein. Such administration can be systemic or local. In some embodiments, a linker polypeptide described herein is administered systemically or locally to treat a cancer.

The following embodiments are encompassed.

Embodiment 1 is a linker polypeptide, comprising:
a first targeting sequence;
a second targeting sequence; and
a first linker between the first targeting sequence and the second targeting sequence, the linker comprising a protease-cleavable polypeptide sequence.

Embodiment 2 is the linker polypeptide of the immediately preceding embodiment, further comprising a first active domain, optionally wherein the first active domain is proximal to the first targeting sequence relative to the second targeting sequence.

Embodiment 3 is the linker polypeptide of the immediately preceding embodiment, further comprising an additional domain, optionally wherein the additional domain comprises an inhibitory polypeptide sequence capable of blocking an activity of the first active domain, a pharmacokinetic modulator, and/or a second active domain, and optionally wherein the additional domain is proximal to the second targeting sequence relative to the first targeting sequence.

Embodiment 4 is the linker polypeptide of the immediately preceding embodiment, comprising sequentially, from the N-terminus to the C-terminus or from the C-terminus to the N-terminus, the first active domain, the first targeting sequence, the first linker, the second targeting sequence, and the additional domain.

Embodiment 5 is a linker polypeptide, comprising
a first active domain;
a second active domain;
a pharmacokinetic modulator; and
a first linker between the pharmacokinetic modulator and the first active domain, the first linker comprising a protease-cleavable polypeptide sequence.

Embodiment 6 is the linker polypeptide of embodiment 5, further comprising a first targeting sequence.

Embodiment 7 is a linker polypeptide, comprising:
a first active domain;
an inhibitory polypeptide sequence capable of blocking an activity of the first active domain;
a first linker between the first active domain and the inhibitory polypeptide sequence, the linker comprising a protease-cleavable polypeptide sequence; and
a first targeting sequence.

Embodiment 8 is the linker polypeptide of the immediately preceding embodiment, comprising a pharmacokinetic modulator.

Embodiment 9 is a linker polypeptide, comprising:
a first polypeptide chain comprising a first active domain, a first domain of a pharmacokinetic modulator, and a first linker between the first active domain and the first domain of the pharmacokinetic modulator, wherein the first active domain is C-terminal to the first domain of the pharmacokinetic modulator;
a second polypeptide chain, comprising a second domain of the pharmacokinetic modulator, an inhibitory polypeptide sequence capable of blocking an activity of the first active domain, and a second linker between the second domain of the pharmacokinetic modulator and the inhibitory polypeptide sequence;
wherein the first linker comprises a protease-cleavable polypeptide sequence; and
the first polypeptide chain or the second polypeptide chain further comprises at least one targeting sequence.

Embodiment 10 is a linker polypeptide, comprising:
a first polypeptide chain comprising a first active domain, a first domain of a pharmacokinetic modulator, and a first linker between the first active domain and the first domain of the pharmacokinetic modulator, wherein the first active domain is N-terminal to the first domain of the pharmacokinetic modulator;
a second polypeptide chain, comprising a second domain of the pharmacokinetic modulator, an inhibitory polypeptide sequence capable of blocking an activity of the first active domain, and a second linker between the second domain of the pharmacokinetic modulator and the inhibitory polypeptide sequence;
wherein the first linker comprises a protease-cleavable polypeptide sequence; and the first polypeptide chain or the second polypeptide chain further comprises at least one targeting sequence.

Embodiment 11 is the linker polypeptide of embodiment 9 or 10, wherein the inhibitory polypeptide sequence is C-terminal to the second domain of the pharmacokinetic modulator.

Embodiment 12 is the linker polypeptide of embodiment 9 or 10, wherein the inhibitory polypeptide sequence is N-terminal to the second domain of the pharmacokinetic modulator.

Embodiment 13 is the linker polypeptide of any one of embodiments 9-12, wherein the targeting sequence is between the protease-cleavable polypeptide sequence and the first domain of the pharmacokinetic modulator.

Embodiment 14 is the linker polypeptide of any one of embodiments 9-12, wherein the targeting sequence is between the protease-cleavable polypeptide sequence and the first active domain.

Embodiment 15 is the linker polypeptide of any one of embodiments 9-12, wherein the targeting sequence is C-terminal to the first active domain.

Embodiment 16 is the linker polypeptide of any one of embodiments 9-12, wherein the targeting sequence is N-terminal to the first active domain.

Embodiment 17 is the linker polypeptide of any one of embodiments 9-12, wherein the targeting sequence is C-terminal to the inhibitory polypeptide sequence.

Embodiment 18 is the linker polypeptide of any one of embodiments 9-12, wherein the targeting sequence is N-terminal to the inhibitory polypeptide sequence.

Embodiment 19 is the linker polypeptide of any one of embodiments 9-12, wherein the targeting sequence is between the inhibitory polypeptide sequence and the second domain of the pharmacokinetic modulator.

Embodiment 20 is the linker polypeptide of any one of embodiments 9-19, wherein the targeting sequence binds heparin, optionally wherein the targeting sequence comprises SEQ ID NO: 664.

Embodiment 21 is the linker polypeptide of any one of embodiments 9-19, wherein the targeting sequence binds collagen IV, optionally wherein the targeting sequence comprises SEQ ID NO: 200.

Embodiment 22 is the linker polypeptide of any one of embodiments 9-19, wherein the targeting sequence binds collagen I, optionally wherein the targeting sequence comprises SEQ ID NO: 188.

Embodiment 23 is the linker polypeptide of any one of embodiments 9-19, wherein the targeting sequence binds fibronectin, optionally wherein the targeting sequence comprises SEQ ID NO: 653.

Embodiment 24 is the linker polypeptide of any one of embodiments 9-23, wherein the targeting sequence is a first targeting sequence and the linker polypeptide further comprises a second targeting sequence.

Embodiment 25 is the linker polypeptide of the immediately preceding embodiment, wherein the first targeting sequence is part of the first polypeptide chain and the second targeting sequence is part of the second polypeptide chain.

Embodiment 26 is the linker polypeptide of the immediately preceding embodiment, wherein the first targeting sequence is C-terminal to the first active domain and the second targeting sequence is C-terminal to the inhibitory polypeptide sequence.

Embodiment 27 is the linker polypeptide of any one of embodiments 24-26, wherein the second targeting sequence binds heparin, optionally wherein the targeting sequence comprises SEQ ID NO: 664.

Embodiment 28 is the linker polypeptide of any one of embodiments 24-26, wherein the second targeting sequence binds collagen IV, optionally wherein the targeting sequence comprises SEQ ID NO: 200.

Embodiment 29 is the linker polypeptide of any one of embodiments 24-26, wherein the second targeting sequence binds collagen I, optionally wherein the targeting sequence comprises SEQ ID NO: 188.

Embodiment 30 is the linker polypeptide of any one of embodiments 24-26, wherein the second targeting sequence binds fibronectin, optionally wherein the targeting sequence comprises SEQ ID NO: 653.

Embodiment 31 is the linker polypeptide of any one of embodiments 9-30, further comprising a second active domain, optionally wherein the second active domain is part of the second polypeptide chain.

Embodiment 32 is the linker polypeptide of any one of embodiments 9-31, wherein the inhibitory polypeptide sequence is a first inhibitory polypeptide sequence, and the linker polypeptide further comprises a second inhibitory polypeptide sequence.

Embodiment 33 is the linker polypeptide of the immediately preceding embodiment, wherein the second inhibitory polypeptide sequence is part of the second polypeptide chain.

Embodiment 34 is the linker polypeptide of the immediately preceding embodiment, wherein the second inhibitory polypeptide sequence is C-terminal to the first inhibitory polypeptide sequence.

Embodiment 35 is the linker polypeptide of any one of embodiments 32-34, wherein the second inhibitory polypeptide sequence is an immunoglobulin inhibitory polypeptide sequence.

Embodiment 36 is the linker polypeptide of the immediately preceding embodiment, wherein the first inhibitory polypeptide sequence is an immunoglobulin inhibitory polypeptide sequence.

Embodiment 37 is the linker polypeptide of embodiment 35 or 36, wherein one or each of the immunoglobulin inhibitory polypeptide sequences is a VHH.

Embodiment 38 is the linker polypeptide of any one of embodiments 8-37, wherein the pharmacokinetic modulator comprises a heterodimeric Fc or heterodimeric CH3 domains.

Embodiment 39 is the linker polypeptide of the immediately preceding embodiment, wherein the heterodimeric Fc or heterodimeric CH3 domains comprise a knob CH3 domain and a hole CH3 domain.

Embodiment 40 is the linker polypeptide of the immediately preceding embodiment, wherein the first domain of the pharmacokinetic modulator is a knob CH3 domain and the second domain of the pharmacokinetic modulator is a hole CH3 domain.

Embodiment 41 is the linker polypeptide of embodiment 39, wherein the first domain of the pharmacokinetic modulator is a hole CH3 domain and the second domain of the pharmacokinetic modulator is a knob CH3 domain.

Embodiment 42 is the linker polypeptide of any one of embodiments 38-41, wherein the pharmacokinetic modulator comprises the sequence of SEQ ID NO: 75.

Embodiment 43 is the linker polypeptide of any one of embodiments 38-41, wherein the pharmacokinetic modulator comprises the sequence of SEQ ID NO: 76.

Embodiment 44 is the linker polypeptide of any one of embodiments 38-41, wherein the pharmacokinetic modulator comprises the sequence of SEQ ID NO: 756.

Embodiment 45 is the linker polypeptide of any one of embodiments 38-44, wherein the pharmacokinetic modulator comprises the sequence of SEQ ID NO: 77.

Embodiment 46 is the linker polypeptide of any one of embodiments 38-44, wherein the pharmacokinetic modulator comprises the sequence of SEQ ID NO: 78.

Embodiment 47 is the linker polypeptide of any one of embodiments 38-44, wherein the pharmacokinetic modulator comprises the sequence of SEQ ID NO: 757.

Embodiment 48 is the linker polypeptide of any one of the preceding embodiments, wherein the first active domain comprises a first immunoglobulin antigen-binding domain.

Embodiment 49 is the linker polypeptide of any one of the preceding embodiments, wherein the second active domain comprises a second immunoglobulin antigen-binding domain.

Embodiment 50 is the linker polypeptide of any one of the preceding embodiments, wherein one or each of the first immunoglobulin antigen-binding domain and the second immunoglobulin antigen-binding domain independently comprises a VH region and a VL region.

Embodiment 51 is the linker polypeptide of any one of the preceding embodiments, wherein one or each of the first immunoglobulin antigen-binding domain and the second immunoglobulin antigen-binding domain independently comprises an Fv, scFv, Fab, or VHH.

Embodiment 52 is the linker polypeptide of any one of the preceding embodiments, wherein one or each of the first immunoglobulin antigen-binding domain and the second immunoglobulin antigen-binding domain independently is humanized or fully human.

Embodiment 53 is the linker polypeptide of any one of the preceding embodiments, wherein one or each of the first immunoglobulin antigen-binding domain and the second immunoglobulin antigen-binding domain independently is configured to bind to one or more sequences selected from a cancer cell surface antigen sequence, a growth factor sequence, and a growth factor receptor sequence.

Embodiment 54 is the linker polypeptide of the immediately preceding embodiment, wherein one or each of the first immunoglobulin antigen-binding domain and the second immunoglobulin antigen-binding domain independently is configured to bind to a HER2 sequence, an EGFR extracellular domain sequence, a PD-1 extracellular domain sequence, a PD-L1 extracellular domain sequence, or a CD3 extracellular domain sequence.

Embodiment 55 is the linker polypeptide of any one of the preceding embodiments, wherein one of the first immunoglobulin antigen-binding domain and the second immunoglobulin antigen-binding domain is configured to bind to a HER2 sequence.

Embodiment 56 is the linker polypeptide of the immediately preceding embodiment, wherein one of the first immunoglobulin antigen-binding domain and the second immunoglobulin antigen-binding domain comprises a VH region comprising hypervariable regions (HVRs) HVR-1, HVR-2, and HVR-3 of a VH region comprising the amino acid sequence of SEQ ID NO: 910, and a VL region comprising HVR-1, HVR-2, and HVR-3 of a VL region comprising the amino acid sequence of SEQ ID NO: 909.

Embodiment 57 is the linker polypeptide of the immediately preceding embodiment, wherein one of the first immunoglobulin antigen-binding domain and the second immunoglobulin antigen-binding domain comprises a VH region comprising the amino acid sequence of SEQ ID NO: 910; and a VL region comprising the amino acid sequence of SEQ ID NO: 909.

Embodiment 58 is the linker polypeptide of embodiment 55 or 56, wherein one of the first immunoglobulin antigen-binding domain and the second immunoglobulin antigen-binding domain comprises a sequence that has at least 80, 85, 90, 95, 97, 98, or 99 percent identity to the sequence of SEQ ID NO: 909 or 910.

Embodiment 59 is the linker polypeptide of embodiment 55, wherein one of the first immunoglobulin antigen-binding domain and the second immunoglobulin antigen-binding domain is an antigen-binding domain of trastuzumab.

Embodiment 60 is the linker polypeptide of any one of the preceding embodiments, wherein one of the first immunoglobulin antigen-binding domain and the second immunoglobulin antigen-binding domain is configured to bind to an EGFR extracellular domain sequence.

Embodiment 61 is the linker polypeptide of the immediately preceding embodiment, wherein one of the first immunoglobulin antigen-binding domain and the second immunoglobulin antigen-binding domain comprises a VH region comprising HVR-1, HVR-2, and HVR-3 of a VH region comprising the amino acid sequence of SEQ ID NO: 914, and a VL region comprising HVR-1, HVR-2, and HVR-3 of a VL region comprising the amino acid sequence of SEQ ID NO: 913.

Embodiment 62 is the linker polypeptide of the immediately preceding embodiment, wherein one of the first immunoglobulin antigen-binding domain and the second immunoglobulin antigen-binding domain comprises a VH region comprising the amino acid sequence of SEQ ID NO: 914; and a VL region comprising the amino acid sequence of SEQ ID NO: 913.

Embodiment 63 is the linker polypeptide of embodiment 60 or 61, wherein one of the first immunoglobulin antigen-binding domain and the second immunoglobulin antigen-binding domain comprises a sequence that has at least 80, 85, 90, 95, 97, 98, or 99 percent identity to the sequence of SEQ ID NO: 913 or 914.

Embodiment 64 is the linker polypeptide of embodiment 60, wherein one of the first immunoglobulin antigen-binding domain and the second immunoglobulin antigen-binding domain is an antigen-binding domain of cetuximab.

Embodiment 65 is the linker polypeptide of any one of the preceding embodiments, wherein one of the first immunoglobulin antigen-binding domain and the second immunoglobulin antigen-binding domain is configured to bind to a PD-1 extracellular domain sequence.

Embodiment 66 is the linker polypeptide of the immediately preceding embodiment, wherein one of the first immunoglobulin antigen-binding domain and the second immunoglobulin antigen-binding domain comprises a VH region comprising HVR-1, HVR-2, and HVR-3 of a VH region comprising the amino acid sequence of SEQ ID NO: 917, and a VL region comprising HVR-1, HVR-2, and HVR-3 of a VL region comprising the amino acid sequence of SEQ ID NO: 918.

Embodiment 67 is the linker polypeptide of the immediately preceding embodiment, wherein one of the first immunoglobulin antigen-binding domain and the second immunoglobulin antigen-binding domain comprises a VH region comprising the amino acid sequence of SEQ ID NO: 917; and a VL region comprising the amino acid sequence of SEQ ID NO: 918.

Embodiment 68 is the linker polypeptide of embodiment 65 or 66, wherein one of the first immunoglobulin antigen-binding domain and the second immunoglobulin antigen-binding domain comprises a sequence that has at least 80, 85, 90, 95, 97, 98, or 99 percent identity to the sequence of SEQ ID NO: 917 or 918.

Embodiment 69 is the linker polypeptide of embodiment 65, wherein one of the first immunoglobulin antigen-binding domain and the second immunoglobulin antigen-binding domain is an antigen-binding domain of nivolumab.

Embodiment 70 is the linker polypeptide of any one of the preceding embodiments, wherein one of the first immunoglobulin antigen-binding domain and the second immunoglobulin antigen-binding domain is configured to bind to a PD-L1 extracellular domain sequence.

Embodiment 71 is the linker polypeptide of the immediately preceding embodiment, wherein one of the first immunoglobulin antigen-binding domain and the second immunoglobulin antigen-binding domain comprises a VH region comprising HVR-1, HVR-2, and HVR-3 of a VH region comprising the amino acid sequence of SEQ ID NO: 921, and a VL region comprising HVR-1, HVR-2, and HVR-3 of a VL region comprising the amino acid sequence of SEQ ID NO: 922.

Embodiment 72 is the linker polypeptide of the immediately preceding embodiment, wherein one of the first immunoglobulin antigen-binding domain and the second immunoglobulin antigen-binding domain comprises a VH region comprising the amino acid sequence of SEQ ID NO: 921; and a VL region comprising the amino acid sequence of SEQ ID NO: 922.

Embodiment 73 is the linker polypeptide of embodiment 70 or 71, wherein one of the first immunoglobulin antigen-binding domain and the second immunoglobulin antigen-binding domain comprises a sequence that has at least 80, 85, 90, 95, 97, 98, or 99 percent identity to the sequence of SEQ ID NO: 921 or 922.

Embodiment 74 is the linker polypeptide of embodiment 70, wherein one of the first immunoglobulin antigen-binding domain and the second immunoglobulin antigen-binding domain is an antigen-binding domain of atezolizumab.

Embodiment 75 is the linker polypeptide of any one of the preceding embodiments, wherein one of the first immunoglobulin antigen-binding domain and the second immunoglobulin antigen-binding domain is configured to bind to a CD3 extracellular domain sequence.

Embodiment 76 is the linker polypeptide of the immediately preceding embodiment, wherein one of the first immunoglobulin antigen-binding domain and the second immunoglobulin antigen-binding domain comprises a VH region comprising HVR-1, HVR-2, and HVR-3 of a VH region comprising the amino acid sequence of any one of SEQ ID NOs: 925, 929, 933, and 937, and a VL region comprising HVR-1, HVR-2, and HVR-3 of a VL region comprising the amino acid sequence of any one of SEQ ID NOs: 926, 930, 934, and 938.

Embodiment 77 is the linker polypeptide of the immediately preceding embodiment, wherein one of the first immunoglobulin antigen-binding domain and the second immunoglobulin antigen-binding domain comprises a VH region comprising the amino acid sequence of any one of SEQ ID NOs: 925, 929, 933, and 937; and a VL region comprising the amino acid sequence of any one of SEQ ID NOs: 926, 930, 934, and 938.

Embodiment 78 is the linker polypeptide of embodiment 75 or 76, wherein one of the first immunoglobulin antigen-binding domain and the second immunoglobulin antigen-binding domain comprises a sequence that has at least 80, 85, 90, 95, 97, 98, or 99 percent identity to the sequence of any one of SEQ ID NOs: 925, 926, 929, 930, 933, 934, 937, and 938.

Embodiment 79 is the linker polypeptide of embodiment 75, wherein one of the first immunoglobulin antigen-binding domain and the second immunoglobulin antigen-binding domain is an antigen-binding domain of teplizumab, muromonab, otelixizumab, or visilizumab.

Embodiment 80 is the linker polypeptide of any one of the preceding embodiments, wherein the first active domain comprises a receptor-binding domain.

Embodiment 81 is the linker polypeptide of the immediately preceding embodiment, wherein the receptor-binding domain comprises a cytokine polypeptide sequence.

Embodiment 82 is the linker polypeptide of any one of embodiments 80-81, wherein the receptor-binding domain comprises a modification to prevent disulfide bond formation, and optionally otherwise comprises wild-type sequence.

Embodiment 83 is the linker polypeptide of any one of embodiments 80-82, wherein the receptor-binding domain has at least 80, 85, 90, 95, 97, 98, or 99 percent identity to the sequence of a wild-type receptor-binding domain or to a receptor-binding domain in Table 1.

Embodiment 84 is the linker polypeptide of the immediately preceding embodiment, wherein the receptor-binding domain is a wild-type receptor-binding domain.

Embodiment 85 is the linker polypeptide of any one of embodiments 80-84, wherein the receptor-binding domain is a monomeric cytokine, or wherein the receptor-binding domain is a dimeric receptor-binding domain comprising monomers that are associated covalently (optionally via a polypeptide linker) or noncovalently.

Embodiment 86 is the linker polypeptide of any one of embodiments 80-85, further comprising
an inhibitory polypeptide sequence capable of blocking an activity of the receptor-binding domain; and
a second linker between the receptor-binding domain and the inhibitory polypeptide sequence, the second linker comprising a protease-cleavable polypeptide sequence.

Embodiment 87 is the linker polypeptide of any one of embodiments 80-86 insofar as they depend from any one of embodiments 9-24, wherein the inhibitory polypeptide sequence comprises a cytokine-binding domain.

Embodiment 88 is the linker polypeptide of any one of embodiments 9-47 or 86-87, wherein the inhibitory polypeptide sequence comprises a cytokine-binding domain.

Embodiment 89 is the linker polypeptide of embodiment 87 or 88, wherein the cytokine-binding domain is a cytokine-binding domain of a cytokine receptor or a cytokine-binding domain of a fibronectin.

Embodiment 90 is the linker polypeptide of the immediately preceding embodiment, wherein the cytokine-binding domain is an immunoglobulin cytokine-binding domain.

Embodiment 91 is the linker polypeptide of the immediately preceding embodiment, wherein the immunoglobulin cytokine-binding domain comprises a VL region and a VH region that bind the cytokine.

Embodiment 92 is the linker polypeptide of embodiment 90 or 91, wherein the immunoglobulin cytokine-binding domain is an Fv, scFv, Fab, or VHH.

Embodiment 93 is the linker polypeptide of any one of embodiments 80-92, comprising a targeting sequence, wherein the targeting sequence is between the receptor-binding domain and the protease-cleavable polypeptide sequence or one of the protease-cleavable polypeptide sequences.

Embodiment 94 is the linker polypeptide of any one of embodiments 80-93, wherein the receptor-binding domain is an interleukin polypeptide sequence.

Embodiment 95 is the linker polypeptide of any one of embodiments 80-94, wherein the receptor-binding domain is capable of binding a receptor comprising CD132.

Embodiment 96 is the linker polypeptide of any one of embodiments 80-95, wherein the receptor-binding domain is capable of binding a receptor comprising CD122.

Embodiment 97 is the linker polypeptide of any one of embodiments 80-96, wherein the receptor-binding domain is capable of binding a receptor comprising CD25.

Embodiment 98 is the linker polypeptide of any one of embodiments 80-97, wherein the receptor-binding domain is capable of binding a receptor comprising IL-10R.

Embodiment 99 is the linker polypeptide of any one of embodiments 80-98, wherein the receptor-binding domain is capable of binding a receptor comprising IL-15R.

Embodiment 100 is the linker polypeptide of any one of embodiments 80-99, wherein the receptor-binding domain is capable of binding a receptor comprising CXCR3.

Embodiment 101 is the linker polypeptide of any one of embodiments 80-100, wherein the receptor-binding domain is an IL-2 polypeptide sequence.

Embodiment 102 is the linker polypeptide of the immediately preceding embodiment, wherein the IL-2 polypeptide sequence has at least 80, 85, 90, 95, 97, 98, or 99 percent identity to the sequence of any one of SEQ ID NOs: 1-4.

Embodiment 103 is the linker polypeptide of the immediately preceding embodiment, wherein the IL-2 polypeptide sequence comprises the sequence of any one of SEQ ID NOs: 1-4.

Embodiment 104 is the linker polypeptide of any one of embodiments 101-103, wherein the IL-2 polypeptide sequence is a human IL-2 polypeptide sequence.

Embodiment 105 is the linker polypeptide of the immediately preceding embodiment, wherein the IL-2 polypeptide sequence comprises the sequence of SEQ ID NO: 1.

Embodiment 106 is the linker polypeptide of any one of embodiments 101-104, wherein the IL-2 polypeptide sequence comprises the sequence of SEQ ID NO: 2.

Embodiment 107 is the linker polypeptide of any one of the preceding embodiments, wherein the inhibitory polypeptide sequence comprises an IL-2 binding domain of an IL-2 receptor (IL-2R).

Embodiment 108 is the linker polypeptide of the immediately preceding embodiment, wherein the inhibitory polypeptide sequence comprises an amino acid sequence having at least 80, 85, 90, 95, 97, 98, or 99 percent identity to the sequence of any one of SEQ ID NOs: 10-29 and 40-51.

Embodiment 109 is the linker polypeptide of embodiment 107 or 108, wherein the IL-2R is a human IL-2R.

Embodiment 110 is the linker polypeptide of any one of the preceding embodiments, wherein the inhibitory polypeptide sequence comprises an IL-2-binding immunoglobulin domain.

Embodiment 111 is the linker polypeptide of the immediately preceding embodiment, wherein the IL-2-binding immunoglobulin domain is a human IL-2-binding immunoglobulin domain.

Embodiment 112 is the linker polypeptide of embodiment 110 or 111, wherein the IL-2-binding immunoglobulin domain comprises a VH region comprising hypervariable regions (HVRs) HVR-1, HVR-2, and HVR-3 having the sequences of SEQ ID NOs: 37, 38, and 39, respectively, and a VL region comprising HVR-1, HVR-2, and HVR-3 having the sequences of SEQ ID NOs: 34, 35, and 36, respectively.

Embodiment 113 is the linker polypeptide of any one of embodiments 110-112, wherein the IL-2-binding immunoglobulin domain comprises a VH region comprising an amino acid sequence having at least 80, 85, 90, 95, 97, 98, or 99 percent identity to the sequence of SEQ ID NO: 33 and a VL region comprising an amino acid sequence having at least 80, 85, 90, 95, 97, 98, or 99 percent identity to the sequence of SEQ ID NO: 32, or a VH region comprising an amino acid sequence having at least 80, 85, 90, 95, 97, 98, or 99 percent identity to the sequence of SEQ ID NO: 749 and a VL region comprising an amino acid sequence having at least 80, 85, 90, 95, 97, 98, or 99 percent identity to the sequence of SEQ ID NO: 748.

Embodiment 114 is the linker polypeptide of the immediately preceding embodiment, wherein the IL-2-binding immunoglobulin domain comprises a VH region comprising the sequence of SEQ ID NO: 33 and a VL region comprising the sequence of SEQ ID NO: 32, or a VH region comprising the sequence of SEQ ID NO: 749 and a VL region comprising the sequence of SEQ ID NO: 748.

Embodiment 115 is the linker polypeptide of any one of embodiments 110-114, wherein the IL-2-binding immunoglobulin domain is an scFv.

Embodiment 116 is the linker polypeptide of embodiment 110, 111, or 114, wherein the IL-2-binding immunoglobulin domain comprises the CDRs of an amino acid sequence of SEQ ID NO: 30, 31, 747, 850-856, or 863-870.

Embodiment 117 is the linker polypeptide of embodiment 110, 111, 114, or 116, wherein the IL-2-binding immunoglobulin domain comprises an amino acid sequence having at least 80, 85, 90, 95, 97, 98, or 99 percent identity to the sequence of SEQ ID NO: 30, 31, 747, 850-856, or 863-870.

Embodiment 118 is the linker polypeptide of the immediately preceding embodiment, wherein the IL-2-binding immunoglobulin domain comprises the sequence of SEQ ID NO: 30, 31, 747, 850-856, or 863-870.

Embodiment 119 is the linker polypeptide of any one of the preceding embodiments, wherein the receptor-binding domain is an IL-10 polypeptide sequence.

Embodiment 120 is the linker polypeptide of the immediately preceding embodiment, wherein the IL-10 polypeptide sequence has at least 80, 85, 90, 95, 97, 98, or 99 percent identity to the sequence of SEQ ID NO: 900.

Embodiment 121 is the linker polypeptide of the immediately preceding embodiment, wherein the IL-10 polypeptide sequence comprises the sequence of SEQ ID NO: 900.

Embodiment 122 is the linker polypeptide of any one of embodiments 119-121, wherein the IL-10 polypeptide sequence is a human IL-10 polypeptide sequence.

Embodiment 123 is the linker polypeptide of any one of embodiments 118-122, wherein the inhibitory polypeptide sequence comprises an IL-10 binding domain of an IL-10 receptor (IL-10R).

Embodiment 124 is the linker polypeptide of the immediately preceding embodiment, wherein the inhibitory polypeptide sequence comprises an amino acid sequence having at least 80, 85, 90, 95, 97, 98, or 99 percent identity to the sequence of SEQ ID NO: 1011 or 1012.

Embodiment 125 is the linker polypeptide of embodiment 123 or 124, wherein the IL-10R is a human IL-10R.

Embodiment 126 is the linker polypeptide of any one of the preceding embodiments, wherein the inhibitory polypeptide sequence comprises an IL-10-binding immunoglobulin domain.

Embodiment 127 is the linker polypeptide of the immediately preceding embodiment, wherein the IL-O-binding immunoglobulin domain is a human IL-O-binding immunoglobulin domain.

Embodiment 128 is the linker polypeptide of embodiment 126 or 127, wherein the IL-10-binding immunoglobulin domain comprises a VH region comprising hypervariable regions (HVRs) HVR-1, HVR-2, and HVR-3 having the sequences of SEQ ID NOs: 946, 947, and 948, respectively, and a VL region comprising HVR-1, HVR-2, and HVR-3 having the sequences of SEQ ID NOs: 942, 943, and 944, respectively.

Embodiment 129 is the linker polypeptide of any one of embodiments 126-128, wherein the IL-10-binding immunoglobulin domain comprises a VH region comprising an amino acid sequence having at least 80, 85, 90, 95, 97, 98, or 99 percent identity to the sequence of SEQ ID NO: 945 and a VL region comprising an amino acid sequence having at least 80, 85, 90, 95, 97, 98, or 99 percent identity to the sequence of SEQ ID NO: 941.

Embodiment 130 is the linker polypeptide of the immediately preceding embodiment, wherein the IL-10-binding immunoglobulin domain comprises a VH region comprising the sequence of SEQ ID NO: 945 and a VL region comprising the sequence of SEQ ID NO: 941.

Embodiment 131 is the linker polypeptide of any one of embodiments 126-130, wherein the IL-10-binding immunoglobulin domain is an scFv.

Embodiment 132 is the linker polypeptide of the immediately preceding embodiment, wherein the IL-10-binding immunoglobulin domain comprises an amino acid sequence having at least 80, 85, 90, 95, 97, 98, or 99 percent identity to the sequence of SEQ ID NO: 939 or 940.

Embodiment 133 is the linker polypeptide of the immediately preceding embodiment, wherein the IL-10-binding immunoglobulin domain comprises the sequence of SEQ ID NO: 939 or 940.

Embodiment 134 is the linker polypeptide of any one of the preceding embodiments, wherein the receptor-binding domain is an IL-15 polypeptide sequence.

Embodiment 135 is the linker polypeptide of the immediately preceding embodiment, wherein the IL-15 polypeptide sequence has at least 80, 85, 90, 95, 97, 98, or 99 percent identity to the sequence of SEQ ID NO: 901.

Embodiment 136 is the linker polypeptide of the immediately preceding embodiment, wherein the IL-15 polypeptide sequence comprises the sequence of SEQ ID NO: 901.

Embodiment 137 is the linker polypeptide of any one of embodiments 134-136, wherein the IL-15 polypeptide sequence is a human IL-15 polypeptide sequence.

Embodiment 138 is the linker polypeptide of any one of embodiments 133-137, wherein the inhibitory polypeptide sequence comprises an IL-15 binding domain of an IL-15 receptor (IL-15R).

Embodiment 139 is the linker polypeptide of the immediately preceding embodiment, wherein the inhibitory polypeptide sequence comprises an amino acid sequence having at least 80, 85, 90, 95, 97, 98, or 99 percent identity to the sequence of any one of SEQ ID NOs: 1016-1019.

Embodiment 140 is the linker polypeptide of embodiment 97 or 98, wherein the IL-15R is a human IL-15R.

Embodiment 141 is the linker polypeptide of any one of the preceding embodiments, wherein the inhibitory polypeptide sequence comprises an IL-15-binding immunoglobulin domain.

Embodiment 142 is the linker polypeptide of the immediately preceding embodiment, wherein the IL-15-binding immunoglobulin domain is a human IL-15-binding immunoglobulin domain.

Embodiment 143 is the linker polypeptide of embodiment 141 or 142, wherein the IL-15-binding immunoglobulin domain comprises a VH region comprising HVR-1, HVR-2, and HVR-3 of a VH region comprising the amino acid sequence of any one of SEQ ID NOs: 950, 955, 957, 960, 963, 966, 969, 972, 975, 978, 981, 985, and 988, and a VL region comprising HVR-1, HVR-2, and HVR-3 of a VL region comprising the amino acid sequence of any one of SEQ ID NOs: 952, 954, 958, 961, 964, 967, 970, 973, 976, 979, 982, 984, and 987.

Embodiment 144 is the linker polypeptide of any one of embodiments 141-143, wherein the IL-15-binding immunoglobulin domain comprises a VH region comprising an amino acid sequence having at least 80, 85, 90, 95, 97, 98, or 99 percent identity to the sequence of any one of SEQ ID NOs: 950, 955, 957, 960, 963, 966, 969, 972, 975, 978, 981, 985, and 988 and a VL region comprising an amino acid sequence having at least 80, 85, 90, 95, 97, 98, or 99 percent identity to the sequence of any one of SEQ ID NOs: 952, 954, 958, 961, 964, 967, 970, 973, 976, 979, 982, 984, and 987.

Embodiment 145 is the linker polypeptide of the immediately preceding embodiment, wherein the IL-15-binding immunoglobulin domain comprises a VH region comprising the sequence of any one of SEQ ID NOs: 950, 955, 957, 960, 963, 966, 969, 972, 975, 978, 981, 985, and 988 and a VL region comprising the sequence of any one of SEQ ID NOs: 952, 954, 958, 961, 964, 967, 970, 973, 976, 979, 982, 984, and 987.

Embodiment 146 is the linker polypeptide of any one of embodiments 141-145, wherein the IL-15-binding immunoglobulin domain is an scFv.

Embodiment 147 is the linker polypeptide of the immediately preceding embodiment, wherein the IL-15-binding immunoglobulin domain comprises an amino acid sequence having at least 80, 85, 90, 95, 97, 98, or 99 percent identity to the sequence of any one of SEQ ID NOs: 953, 956, 959, 962, 965, 968, 971, 974, 977, 980, 983, and 986.

Embodiment 148 is the linker polypeptide of the immediately preceding embodiment, wherein the IL-15-binding immunoglobulin domain comprises the sequence of any one of SEQ ID NOs: 953, 956, 959, 962, 965, 968, 971, 974, 977, 980, 983, and 986.

Embodiment 149 is the linker polypeptide of any one of the preceding embodiments, wherein the receptor-binding domain is an CXCL9 polypeptide sequence.

Embodiment 150 is the linker polypeptide of the immediately preceding embodiment, wherein the CXCL9 polypeptide sequence has at least 80, 85, 90, 95, 97, 98, or 99 percent identity to the sequence of SEQ ID NO: 902.

Embodiment 151 is the linker polypeptide of the immediately preceding embodiment, wherein the CXCL9 polypeptide sequence comprises the sequence of SEQ ID NO: 902.

Embodiment 152 is the linker polypeptide of any one of embodiments 149-150, wherein the CXCL9 polypeptide sequence is a human CXCL9 polypeptide sequence.

Embodiment 153 is the linker polypeptide of any one of embodiments 148-152, wherein the inhibitory polypeptide sequence comprises a CXCL9 binding domain of CXCR3.

Embodiment 154 is the linker polypeptide of the immediately preceding embodiment, wherein the inhibitory polypeptide sequence comprises an amino acid sequence having at least 80, 85, 90, 95, 97, 98, or 99 percent identity to the sequence of SEQ ID NO: 1020 or 1021.

Embodiment 155 is the linker polypeptide of embodiment 153 or 154, wherein the CXCR3 is a human CXCR3.

Embodiment 156 is the linker polypeptide of any one of the preceding embodiments, wherein the inhibitory polypeptide sequence comprises an CXCL9-binding immunoglobulin domain.

Embodiment 157 is the linker polypeptide of the immediately preceding embodiment, wherein the CXCL9-binding immunoglobulin domain is a human CXCL9-binding immunoglobulin domain.

Embodiment 158 is the linker polypeptide of any one of the preceding embodiments, wherein the receptor-binding domain is an CXCL10 polypeptide sequence.

Embodiment 159 is the linker polypeptide of the immediately preceding embodiment, wherein the CXCL10 polypeptide sequence has at least 80, 85, 90, 95, 97, 98, or 99 percent identity to the sequence of SEQ ID NO: 903.

Embodiment 160 is the linker polypeptide of the immediately preceding embodiment, wherein the CXCL10 polypeptide sequence comprises the sequence of SEQ ID NO: 903.

Embodiment 161 is the linker polypeptide of any one of embodiments 158-160, wherein the CXCL10 polypeptide sequence is a human CXCL10 polypeptide sequence.

Embodiment 162 is the linker polypeptide of any one of embodiments 156-161, wherein the inhibitory polypeptide sequence comprises an CXCL10 binding domain of CXCR3.

Embodiment 163 is the linker polypeptide of the immediately preceding embodiment, wherein the inhibitory polypeptide sequence comprises an amino acid sequence having at least 80, 85, 90, 95, 97, 98, or 99 percent identity to the sequence of SEQ ID NO: 1020 or 1021.

Embodiment 164 is the linker polypeptide of embodiment 162 or 163, wherein the CXCR3 is a human CXCR3.

Embodiment 165 is the linker polypeptide of any one of the preceding embodiments, wherein the inhibitory polypeptide sequence comprises an CXCL10-binding immunoglobulin domain.

Embodiment 166 is the linker polypeptide of the immediately preceding embodiment, wherein the CXCL10-binding immunoglobulin domain is a human CXCL10-binding immunoglobulin domain.

Embodiment 167 is the linker polypeptide of embodiment 165 or 166, wherein the CXCL10-binding immunoglobulin domain comprises a VH region comprising hypervariable regions (HVRs) HVR-1, HVR-2, and HVR-3 having the sequences of SEQ ID NOs: 993, 994, and 995, respectively, and a VL region comprising HVR-1, HVR-2, and HVR-3 having the sequences of SEQ ID NOs: 996, 997, and 998, respectively.

Embodiment 168 is the linker polypeptide of any one of embodiments 165-167, wherein the CXCL10-binding immunoglobulin domain comprises a VH region comprising an amino acid sequence having at least 80, 85, 90, 95, 97, 98, or 99 percent identity to the sequence of SEQ ID NO: 991 and a VL region comprising an amino acid sequence having at least 80, 85, 90, 95, 97, 98, or 99 percent identity to the sequence of SEQ ID NO: 992.

Embodiment 169 is the linker polypeptide of the immediately preceding embodiment, wherein the CXCL10-binding immunoglobulin domain comprises a VH region comprising the sequence of SEQ ID NO: 991 and a VL region comprising the sequence of SEQ ID NO: 992.

Embodiment 170 is the linker polypeptide of any one of embodiments 165-169, wherein the CXCL10-binding immunoglobulin domain is an scFv.

Embodiment 171 is the linker polypeptide of the immediately preceding embodiment, wherein the CXCL10-binding immunoglobulin domain comprises an amino acid sequence having at least 80, 85, 90, 95, 97, 98, or 99 percent identity to the sequence of SEQ ID NO: 989 or 990.

Embodiment 172 is the linker polypeptide of the immediately preceding embodiment, wherein the CXCL10-binding immunoglobulin domain comprises the sequence of SEQ ID NO: 989 or 990.

Embodiment 173 is the linker polypeptide of any one of the preceding embodiments, wherein the inhibitory polypeptide sequence interferes with binding between the first active domain and a receptor of the first active domain and/or with binding between the second active domain and a receptor of the second active domain.

Embodiment 174 is the linker polypeptide of any one of the preceding embodiments, wherein the inhibitory polypeptide sequence and the pharmacokinetic modulator are different elements of the linker polypeptide.

Embodiment 175 is the linker polypeptide of any one of the preceding embodiments, wherein the inhibitory polypeptide sequence comprises a steric blocker.

Embodiment 176 is the linker polypeptide of any one of the preceding embodiments, wherein the inhibitory polypeptide sequence comprises at least a portion of the pharmacokinetic modulator.

Embodiment 177 is the linker polypeptide of any one of the preceding embodiments, wherein the pharmacokinetic modulator comprises at least a portion of an immunoglobulin constant domain.

Embodiment 178 is the linker polypeptide of the immediately preceding embodiment, wherein the pharmacokinetic modulator comprises at least a portion of an immunoglobulin Fc region.

Embodiment 179 is the linker polypeptide of the immediately preceding embodiment, wherein the pharmacokinetic modulator comprises an immunoglobulin Fc region.

Embodiment 180 is the linker polypeptide of any one of embodiments 177-179, wherein the immunoglobulin is a human immunoglobulin.

Embodiment 181 is the linker polypeptide of any one of embodiments 177-180, wherein the immunoglobulin is IgG.

Embodiment 182 is the linker polypeptide of the immediately preceding embodiment, wherein the IgG is IgG1, IgG2, IgG3, or IgG4.

Embodiment 183 is the linker polypeptide of any of the preceding embodiments, further comprising a growth factor-binding polypeptide sequence or a growth factor receptor-binding polypeptide sequence.

Embodiment 184 is the linker polypeptide of the immediately preceding embodiment, wherein the growth factor-binding polypeptide sequence comprises a TGF-βR extracellular domain sequence.

Embodiment 185 is the linker polypeptide of the immediately preceding embodiment, wherein the TGF-βR extracellular domain sequence comprises an amino acid sequence having at least 80, 85, 90, 95, 97, 98, or 99 percent identity to the sequence of SEQ ID NO: 1022 or 1023.

Embodiment 186 is the linker polypeptide of the embodiment 142-144, wherein the growth factor-binding polypeptide sequence comprises a growth factor-binding immunoglobulin domain.

Embodiment 187 is the linker polypeptide of the immediately preceding embodiment, wherein the growth factor-binding immunoglobulin domain is configured to bind to a TGF-β.

Embodiment 188 is the linker polypeptide of embodiment 145 or 146, wherein the growth factor-binding immunoglobulin domain comprises a VH region comprising HVR-1, HVR-2, and HVR-3 of a VH region comprising the amino acid sequence of SEQ ID NO: 1008, and a VL region comprising HVR-1, HVR-2, and HVR-3 of a VL region comprising the amino acid sequence of SEQ ID NO: 1010.

Embodiment 189 is the linker polypeptide of the immediately preceding embodiment, wherein the growth factor-binding immunoglobulin domain comprises a VH region comprising the amino acid sequence of SEQ ID NO: 1008; and a VL region comprising the amino acid sequence of SEQ ID NO: 1010.

Embodiment 190 is the linker polypeptide of embodiment 185-189, wherein the growth factor-binding immunoglobulin domain comprises a sequence that has at least 80, 85, 90, 95, 97, 98, or 99 percent identity to the sequence of SEQ ID NO: 1007 or 1009.

Embodiment 191 is the linker polypeptide of embodiment 183-190, wherein the growth factor receptor-binding polypeptide sequence comprises a TGF-β sequence.

Embodiment 192 is the linker polypeptide of the immediately preceding embodiment, wherein the TGF-β sequence comprises an amino acid sequence having at least 80, 85, 90, 95, 97, 98, or 99 percent identity to the sequence of any one of SEQ ID NOs. 904-906.

Embodiment 193 is the linker polypeptide of the embodiment 183-192, wherein the growth factor receptor-binding polypeptide sequence comprises a growth factor receptor-binding immunoglobulin domain.

Embodiment 194 is the linker polypeptide of the immediately preceding embodiment, wherein the growth factor receptor-binding immunoglobulin domain is configured to bind to a TGF-βR extracellular domain sequence.

Embodiment 195 is the linker polypeptide of embodiment 193 or 194, wherein the growth factor receptor-binding immunoglobulin domain comprises a VH region comprising HVR-1, HVR-2, and HVR-3 of a VH region comprising the amino acid sequence of SEQ ID NO: 999 or 1003, and a VL region comprising HVR-1, HVR-2, and HVR-3 of a VL region comprising the amino acid sequence of SEQ ID NO: 1000 or 1004.

Embodiment 196 is the linker polypeptide of the immediately preceding embodiment, wherein the growth factor receptor-binding immunoglobulin domain comprises a VH region comprising the amino acid sequence of SEQ ID NO: 999 or 1003; and a VL region comprising the amino acid sequence of SEQ ID NO: 1000 or 1004.

Embodiment 197 is the linker polypeptide of embodiment 152-155, wherein the growth factor receptor-binding immunoglobulin domain comprises a sequence that has at least 80, 85, 90, 95, 97, 98, or 99 percent identity to the sequence of any one of SEQ ID NOs: 1001, 1002, 1005, and 1006.

Embodiment 198 is the linker polypeptide of any one of the preceding embodiments, comprising a plurality of protease-cleavable polypeptide sequences.

Embodiment 199 is the linker polypeptide of any one of the preceding embodiments, wherein the protease-cleavable polypeptide sequence is C-terminal to a VH region, C-terminal to at least a portion of a CH1 domain, between a CH1 domain and a CH2 domain, N-terminal to at least a portion of a CH2 domain, N-terminal to a disulfide bond between heavy chains, N-terminal to a disulfide bond within a CH2 domain, or N-terminal to a hinge region, or is within a hinge region.

Embodiment 200 is the linker polypeptide of any one of the preceding embodiments, wherein the protease-cleavable polypeptide sequence is C-terminal to the first targeting sequence and to the second targeting sequence.

Embodiment 201 is the linker polypeptide of any one of the preceding embodiments, wherein the protease-cleavable polypeptide sequence is N-terminal to the first targeting sequence and to the second targeting sequence.

Embodiment 202 is the linker polypeptide of any one of the preceding embodiments, wherein the protease-cleavable polypeptide sequence is C-terminal to a first plurality of targeting sequences and is N-terminal to a second plurality of targeting sequences.

Embodiment 203 is the linker polypeptide of any one of the preceding embodiments, wherein the protease-cleavable polypeptide sequence is C-terminal to a plurality of targeting sequences and is N-terminal to at least one targeting sequence.

Embodiment 204 is the linker polypeptide of any one of the preceding embodiments, wherein the protease-cleavable polypeptide sequence is N-terminal to a plurality of targeting sequences and is C-terminal to at least one targeting sequence.

Embodiment 205 is the linker polypeptide of any one of the preceding embodiments, wherein the protease-cleavable polypeptide sequence is C-terminal to the first targeting sequence and to the second targeting sequence and is not N-terminal to a targeting sequence.

Embodiment 206 is the linker polypeptide of any one of the preceding embodiments, wherein the protease-cleavable polypeptide sequence is N-terminal to the first targeting sequence and to the second targeting sequence and is not C-terminal to a targeting sequence.

Embodiment 207 is the linker polypeptide of any one of the preceding embodiments, wherein the linker polypeptide is configured to release the first active domain from a remaining portion of the linker polypeptide upon cleavage of the protease-cleavable polypeptide sequence.

Embodiment 208 is the linker polypeptide of the immediately preceding embodiment, wherein the first active domain is configured to remain connected to one or more of: one of the first targeting sequence and the second targeting sequence, one of the at least one targeting sequence, one of the first plurality of targeting sequences, one of the second plurality of targeting sequences, one of the plurality of targeting sequences, and the pharmacokinetic modulator upon cleavage of the protease-cleavable polypeptide sequence.

Embodiment 209 is the linker polypeptide of any one of the preceding embodiments, wherein the linker polypeptide is configured to release the second active domain from a remaining portion of the linker polypeptide upon cleavage of the protease-cleavable polypeptide sequence.

Embodiment 210 is the linker polypeptide of the immediately preceding embodiment, wherein the second active domain is configured to remain connected to one or more of: one of the first targeting sequence and the second targeting sequence, one of the at least one targeting sequence, one of the first plurality of targeting sequences, one of the second plurality of targeting sequences, one of the plurality of targeting sequences, and the pharmacokinetic modulator upon cleavage of the protease-cleavable polypeptide sequence.

Embodiment 211 is the linker polypeptide of any one of the preceding embodiments, wherein the protease-cleavable polypeptide sequence is recognized by a metalloprotease, a serine protease, a cysteine protease, an aspartate protease, a threonine protease, a glutamate protease, a gelatinase, an asparagine peptide lyase, a cathepsin, a kallikrein, a plasmin, a collagenase, a hK1, a hK10, a hK15, a stromelysin, a Factor Xa, a chymotrypsin-like protease, a trypsin-like protease, a elastase-like protease, a subtilisin-like protease, an actinidain, a bromelain, a calpain, a caspase, a Mir 1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, an ADAM 10, an ADAM 17, an ADAM 12, an urokinase plasminogen activator (uPA), an enterokinase, a prostate-specific target (PSA, hK3), an interleukin-1b converting enzyme, a thrombin, a FAP (FAP-a), a dipeptidyl peptidase, or dipeptidyl peptidase IV (DPPIV/CD26), a type II transmembrane serine protease (TTSP), a neutrophil elastase, a proteinase 3, a mast cell chymase, a mast cell tryptase, or a dipeptidyl peptidase.

Embodiment 212 is the linker polypeptide of any one of the preceding embodiments, wherein the protease-cleavable polypeptide sequence comprises the sequence of any one of SEQ ID NOs: 701-742, or a variant having one or two mismatches relative to the sequence of any one of SEQ ID NOs: 701-742.

Embodiment 213 is the linker polypeptide of any one of the preceding embodiments, wherein the protease-cleavable polypeptide sequence is recognized by a matrix metalloprotease.

Embodiment 214 is the linker polypeptide of any one of the preceding embodiments, wherein the protease-cleavable polypeptide sequence is recognized by MMP-1.

Embodiment 215 is the linker polypeptide of any one of the preceding embodiments, wherein the protease-cleavable polypeptide sequence is recognized by MMP-2.

Embodiment 216 is the linker polypeptide of any one of the preceding embodiments, wherein the protease-cleavable polypeptide sequence is recognized by MMP-3.

Embodiment 217 is the linker polypeptide of any one of the preceding embodiments, wherein the protease-cleavable polypeptide sequence is recognized by MMP-7.

Embodiment 218 is the linker polypeptide of any one of the preceding embodiments, wherein the protease-cleavable polypeptide sequence is recognized by MMP-8.

Embodiment 219 is the linker polypeptide of any one of the preceding embodiments, wherein the protease-cleavable polypeptide sequence is recognized by MMP-9.

Embodiment 220 is the linker polypeptide of any one of the preceding embodiments, wherein the protease-cleavable polypeptide sequence is recognized by MMP-12.

Embodiment 221 is the linker polypeptide of any one of the preceding embodiments, wherein the protease-cleavable polypeptide sequence is recognized by MMP-13.

Embodiment 222 is the linker polypeptide of any one of the preceding embodiments, wherein the protease-cleavable polypeptide sequence is recognized by MMP-14.

Embodiment 223 is the linker polypeptide of any one of the preceding embodiments, wherein the protease-cleavable polypeptide sequence is recognized by more than one MMP.

Embodiment 224 is the linker polypeptide of any one of the preceding embodiments, wherein the protease-cleavable polypeptide sequence is recognized by two, three, four, five, six, or seven of MMP-2, MMP-7, MMP-8, MMP-9, MMP-12, MMP-13, and MMP-14.

Embodiment 225 is the linker polypeptide of any one of the preceding embodiments, wherein the protease-cleavable polypeptide sequence comprises the sequence of any one of SEQ ID NOs: 80-94 or a variant sequence having one or two mismatches relative to the sequence of any one of SEQ ID NOs: 80-90.

Embodiment 226 is the linker polypeptide of any one of the preceding embodiments, wherein the protease-cleavable polypeptide sequence comprises the sequence of SEQ ID NO: 80 or a variant sequence having one or two mismatches relative thereto.

Embodiment 227 is the linker polypeptide of any one of embodiments 1-225, wherein the protease-cleavable polypeptide sequence comprises the sequence of SEQ ID NO: 81 or a variant sequence having one or two mismatches relative thereto.

Embodiment 228 is the linker polypeptide of any one of embodiments 1-225, wherein the protease-cleavable polypeptide sequence comprises the sequence of SEQ ID NO: 82 or a variant sequence having one or two mismatches relative thereto.

Embodiment 229 is the linker polypeptide of any one of embodiments 1-225, wherein the protease-cleavable polypeptide sequence comprises the sequence of SEQ ID NO: 83 or a variant sequence having one or two mismatches relative thereto.

Embodiment 230 is the linker polypeptide of any one of embodiments 1-225, wherein the protease-cleavable polypeptide sequence comprises the sequence of SEQ ID NO: 84 or a variant sequence having one or two mismatches relative thereto.

Embodiment 231 is the linker polypeptide of any one of embodiments 1-225, wherein the protease-cleavable polypeptide sequence comprises the sequence of SEQ ID NO: 85 or a variant sequence having one or two mismatches relative thereto.

Embodiment 232 is the linker polypeptide of any one of embodiments 1-225, wherein the protease-cleavable polypeptide sequence comprises the sequence of SEQ ID NO: 86 or a variant sequence having one or two mismatches relative thereto.

Embodiment 233 is the linker polypeptide of any one of embodiments 1-225, wherein the protease-cleavable polypeptide sequence comprises the sequence of SEQ ID NO: 87 or a variant sequence having one or two mismatches relative thereto.

Embodiment 234 is the linker polypeptide of any one of embodiments 1-225, wherein the protease-cleavable polypeptide sequence comprises the sequence of SEQ ID NO: 88 or a variant sequence having one or two mismatches relative thereto.

Embodiment 235 is the linker polypeptide of any one of embodiments 1-225, wherein the protease-cleavable polypeptide sequence comprises the sequence of SEQ ID NO: 89 or a variant sequence having one or two mismatches relative thereto.

Embodiment 236 is the linker polypeptide of any one of embodiments 1-225, wherein the protease-cleavable polypeptide sequence comprises the sequence of SEQ ID NO: 90 or a variant sequence having one or two mismatches relative thereto.

Embodiment 237 is the linker polypeptide of any one of embodiments 1-225, wherein the protease-cleavable polypeptide sequence comprises the sequence of any one of SEQ ID NO: 80-90.

Embodiment 238 is the linker polypeptide of any one of embodiments 1-225, wherein the protease-cleavable polypeptide sequence comprises the sequence of SEQ ID NO: 91.

Embodiment 239 is the linker polypeptide of any one of embodiments 1-225, wherein the protease-cleavable polypeptide sequence comprises the sequence of SEQ ID NO: 92.

Embodiment 240 is the linker polypeptide of any one of embodiments 1-225, wherein the protease-cleavable polypeptide sequence comprises the sequence of SEQ ID NO: 93.

Embodiment 241 is the linker polypeptide of any one of embodiments 1-225, wherein the protease-cleavable polypeptide sequence comprises the sequence of SEQ ID NO: 94.

Embodiment 242 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind an extracellular matrix component, heparin, an integrin, or a syndecan; or is configured to bind, in a pH-sensitive manner, an extracellular matrix component, heparin, IgB (CD79b), an integrin, a cadherin, a heparan sulfate proteoglycan, a syndecan, or a fibronectin; or the targeting sequence comprises the sequence of any one of SEQ ID NOs: 179-665 or a variant having one or two mismatches relative to the sequence of any one of SEQ ID NOs: 179-665.

Embodiment 243 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently comprises the sequence of any one of SEQ ID NOs: 179-665, or a variant having one or two mismatches relative to the sequence of any one of SEQ ID NOs: 179-665.

Embodiment 244 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently comprises the sequence of any one of SEQ ID NOs: 179-665.

Embodiment 245 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently comprises the sequence of any one of SEQ ID NOs: 200, 330, 619, 653, and 663-665, or a variant having one or two mismatches relative to the sequence of any one of SEQ ID NOs: 200, 330, 619, 653, and 663-665.

Embodiment 246 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently comprises the sequence of any one of SEQ ID NOs: 200, 330, 619, 653, and 663-665.

Embodiment 247 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to denatured collagen.

Embodiment 248 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to collagen.

Embodiment 249 is the linker polypeptide of embodiment 247 or 248, wherein the collagen is collagen I.

Embodiment 250 is the linker polypeptide of embodiment 247 or 248, wherein the collagen is collagen II.

Embodiment 251 is the linker polypeptide of embodiment 247 or 248, wherein the collagen is collagen III.

Embodiment 252 is the linker polypeptide of embodiment 247 or 248, wherein the collagen is collagen IV.

Embodiment 253 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to integrin.

Embodiment 254 is the linker polypeptide of the immediately preceding embodiment, wherein the integrin is one or more of α1β1 integrin, α2β1 integrin, α3β1 integrin, α4β1 integrin, α5β1 integrin, α6β1 integrin, α7β1 integrin, α9β1 integrin, α4β7 integrin, αvβ integrin, αvβ5 integrin, αIIbβ3 integrin, αIIIbβ3 integrin, αMβ2 integrin, or αIIbβ3 integrin.

Embodiment 255 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to von Willebrand factor.

Embodiment 256 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to IgB.

Embodiment 257 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to heparin.

Embodiment 258 is the linker polypeptide of any one of the preceding embodiments, wherein the first targeting sequence is configured to bind to heparin and the second targeting sequence is configured to bind to heparin, wherein the first targeting sequence is configured to bind to collagen IV and the second targeting sequence is configured to bind to heparin, or wherein the first targeting sequence is configured to bind to heparin and the second targeting sequence is configured to bind to collagen IV.

Embodiment 259 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to heparin and a syndecan, a heparan sulfate proteoglycan, or an integrin, optionally wherein the integrin is one or more of α1β1 integrin, α2β1 integrin, α3β1 integrin, α4β1 integrin, α5β1 integrin, α6β1 integrin, α7β1 integrin, α9β1 integrin, α4β7 integrin, αvβ integrin, αvβ5 integrin, αIIbβ3 integrin, αIIIbβ3 integrin, αMβ2 integrin, or αIIbβ3 integrin.

Embodiment 260 is the linker polypeptide of the immediately preceding embodiment, wherein the syndecan is one of more of syndecan-1, syndecan-4, and syndecan-2(w).

Embodiment 261 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to a heparan sulfate proteoglycan.

Embodiment 262 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to a sulfated glycoprotein.

Embodiment 263 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to hyaluronic acid.

Embodiment 264 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to fibronectin.

Embodiment 265 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to cadherin.

Embodiment 266 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target in a pH-sensitive manner.

Embodiment 267 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently has a higher affinity for its target at a pH below normal physiological pH than at normal physiological pH, optionally wherein the pH below normal physiological pH is below 7, or below 6.

Embodiment 268 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently has a higher affinity for its target at a pH in the range of 5-7, e.g., 5-5.5, 5.5-6, 6-6.5, or 6.5-7, than at normal physiological pH.

Embodiment 269 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently comprises one or more histidines, e.g., 1, 2, 3, 4, 5, 6, 7, 8, 9, or 10 histidines.

Embodiment 270 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently comprises the sequence of any one of SEQ ID NOs: 641-663, or a variant having one or two mismatches relative to the sequence of any one of SEQ ID NOs: 641-663.

Embodiment 271 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently comprises the sequence of any one of SEQ ID NOs: 641-665.

Embodiment 272 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind, in a pH-sensitive manner, an extracellular matrix component, IgB (CD79b), an integrin, a cadherin, a heparan sulfate proteoglycan, a syndecan, or a fibronectin.

Embodiment 273 is the linker polypeptide of the immediately preceding embodiment, wherein the extracellular matrix component is hyaluronic acid, heparin, heparan sulfate, or a sulfated glycoprotein.

Embodiment 274 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind a fibronectin in a pH-sensitive manner.

Embodiment 275 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target with an affinity from 0.1 nM to 1 nM, from 1 nM to 10 nM, from 10 nM to 100 nM, from 100 nM to 1 μM, from 1 μM to 10 μM, or from 10 μM to 100 μM.

Embodiment 276 is the linker polypeptide of the immediately preceding embodiment, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target with an affinity from 0.1 nM to 1 nM.

Embodiment 277 is the linker polypeptide of embodiment 275, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target with an affinity from 1 nM to 10 nM.

Embodiment 278 is the linker polypeptide of embodiment 275, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target with an affinity from 10 nM to 100 nM.

Embodiment 279 is the linker polypeptide of embodiment 275, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target with an affinity from 100 nM to 1 M.

Embodiment 280 is the linker polypeptide of embodiment 275, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target with an affinity from 1 μM to 10 μM.

Embodiment 281 is the linker polypeptide of embodiment 275, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target with an affinity from 10 μM to 100 μM.

Embodiment 282 is the linker polypeptide of any one of the preceding embodiments, wherein at least one of the first linker and the second linker comprises one of the first targeting sequence and the second targeting sequence, one of the at least one targeting sequence, one of the first plurality of targeting sequences, one of the second plurality of targeting sequences, or one of the plurality of targeting sequences.

Embodiment 283 is the linker polypeptide of the immediately preceding embodiment, wherein the protease-cleavable polypeptide sequence comprises one of the first targeting sequence and the second targeting sequence, one of the at least one targeting sequence, one of the first plurality of targeting sequences, one of the second plurality of targeting sequences, or one of the plurality of targeting sequences.

Embodiment 284 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences increases a serum half-life of the linker polypeptide.

Embodiment 285 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences synergistically increases a serum half-life of the linker polypeptide together with the pharmacokinetic modulator or with another one of the first targeting sequence and the second targeting sequence, another one of the at least one targeting sequence, another one of the first plurality of targeting sequences, another one of the second plurality of targeting sequences, or another one of the plurality of targeting sequences.

Embodiment 286 is the linker polypeptide of any one of the preceding embodiments, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently increases a serum half-life of the linker polypeptide.

Embodiment 287 is the linker polypeptide of any one of the preceding embodiments, further comprising a blocker conjugated to one of or each of the first active domain and the second active domain.

Embodiment 288 is the linker polypeptide of the immediately preceding embodiment, wherein the blocker is conjugated to one of or each of the first active domain and the second active domain via a protease-cleavable polypeptide sequence.

Embodiment 289 is the linker polypeptide of embodiment 287 or 288, wherein the blocker is an albumin.

Embodiment 290 is the linker polypeptide of any one of embodiments 287-289, wherein the blocker is a serum albumin.

Embodiment 291 is the linker polypeptide of any one of embodiments 287-290, wherein the blocker is a human albumin.

Embodiment 292 is the linker polypeptide of any one of the preceding embodiments, further comprising a chemotherapy drug.

Embodiment 293 is the linker polypeptide of the immediately preceding embodiment, wherein the chemotherapy drug is conjugated to the pharmacokinetic modulator.

Embodiment 294 is the linker polypeptide of embodiment 292 or 293, where the chemotherapy drug is selected from altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, epirubicin, idarubicin, valrubicin, bleomycin, dactinomycin, mitomycin-c, mitoxantrone, irinotecan, topotecan, etoposide, mitoxantrone, teniposide, cabazitaxel, docetaxel, paclitaxel, vinblastine, vincristine, vinorelbine, prednisone, methylprednisolone, dexamethasone, retinoic acid, arsenic trioxide, asparaginase, eribulin, hydroxyurea, ixabepilone, mitotane, omacetaxine, pegaspargase, procarbazine, romidepsin, and vorinostat.

Embodiment 295 is the linker polypeptide of any of the preceding embodiments, wherein a molecular weight of one or each of the first active domain and the second active domain independently is about or less than 14 kDa.

Embodiment 296 is the linker polypeptide of the immediately preceding embodiment, wherein the molecular weight is about 12 kDa to about 14 kDa.

Embodiment 297 is the linker polypeptide of embodiment 295, wherein the molecular weight is about 10 kDa to about 12 kDa.

Embodiment 298 is the linker polypeptide of embodiment 295, wherein the molecular weight is about 8 kDa to about 10 kDa.

Embodiment 299 is the linker polypeptide of embodiment 295, wherein the molecular weight is about 6 kDa to about 8 kDa.

Embodiment 300 is the linker polypeptide of embodiment 295, wherein the molecular weight is about 4 kDa to about 6 kDa.

Embodiment 301 is the linker polypeptide of embodiment 295, wherein the molecular weight is about 2 kDa to about 4 kDa.

Embodiment 302 is the linker polypeptide of embodiment 295, wherein the molecular weight is about 800 Da to about 2 kDa.

Embodiment 303 is the linker polypeptide of any of embodiments 1-294, wherein a molecular weight of one or each of the first active domain and the second active domain independently is about or greater than 16 kDa.

Embodiment 304 is the linker polypeptide of the immediately preceding embodiment, wherein the molecular weight is about 16 kDa to about 18 kDa.

Embodiment 305 is the linker polypeptide of embodiment 303, wherein the molecular weight is about 18 kDa to about 20 kDa.

Embodiment 306 is the linker polypeptide of embodiment 303, wherein the molecular weight is about 20 kDa to about 22 kDa.

Embodiment 307 is the linker polypeptide of embodiment 303, wherein the molecular weight is about 22 kDa to about 24 kDa.

Embodiment 308 is the linker polypeptide of embodiment 303, wherein the molecular weight is about 24 kDa to about 26 kDa.

Embodiment 309 is the linker polypeptide of embodiment 303, wherein the molecular weight is about 26 kDa to about 28 kDa.

Embodiment 310 is the linker polypeptide of embodiment 303, wherein the molecular weight is about 28 kDa to about 30 kDa.

Embodiment 311 is the linker polypeptide of embodiment 303, wherein the molecular weight is about 30 kDa to about 50 kDa.

Embodiment 312 is the linker polypeptide of embodiment 303, wherein the molecular weight is about 50 kDa to about 100 kDa.

Embodiment 313 is the linker polypeptide of embodiment 303, wherein the molecular weight is about 100 kDa to about 150 kDa.

Embodiment 314 is the linker polypeptide of embodiment 303, wherein the molecular weight is about 150 kDa to about 200 kDa.

Embodiment 315 is the linker polypeptide of embodiment 303, wherein the molecular weight is about 200 kDa to about 250 kDa.

Embodiment 316 is the linker polypeptide of embodiment 303, wherein the molecular weight is about 250 kDa to about 300 kDa.

Embodiment 317 is the linker polypeptide of any one of the preceding embodiments, comprising a combined targeting sequence and protease cleavable sequence, wherein the combined targeting sequence and protease cleavable sequence is any one of SEQ ID NOs: 667-673.

Embodiment 318 is a linker polypeptide comprising an amino acid sequence having at least 80, 85, 90, 95, 97, 98, or 99 percent identity to the sequence of any one of SEQ ID NOs: 800-848 or 1024-1041.

Embodiment 319 is the linker polypeptide of the immediately preceding embodiment, comprising the sequence of any one of SEQ ID NOs: 800-848 or 1024-1041.

Embodiment 320 is a pharmaceutical composition comprising the linker polypeptide of any one of the preceding embodiments.

Embodiment 321 is the linker polypeptide or pharmaceutical composition of any one of the preceding embodiments, for use in therapy.

Embodiment 322 is the linker polypeptide or pharmaceutical composition of any one of the preceding embodiments, for use in treating a cancer.

Embodiment 323 is a method of treating a cancer, comprising administering the linker polypeptide or pharmaceutical composition of any one of the preceding embodiments to a subject in need thereof.

Embodiment 324 is use of the linker polypeptide or pharmaceutical composition of any one of embodiments 1-321 for the manufacture of a medicament for treating cancer.

Embodiment 325 is the method, use, or linker polypeptide for use of any one of embodiments 322-324, wherein the cancer is a solid tumor.

Embodiment 326 is the method, use, or linker polypeptide for use of the immediately preceding embodiment, wherein the solid tumor is metastatic and/or unresectable.

Embodiment 327 is the method, use, or linker polypeptide for use of any one of embodiments 322-326, wherein the cancer is a PD-L1-expressing cancer.

Embodiment 328 is the method, use, or linker polypeptide for use of any one of embodiments 322-327, wherein the cancer is a melanoma, a colorectal cancer, a breast cancer, a pancreatic cancer, a lung cancer, a prostate cancer, an ovarian cancer, a cervical cancer, a gastric or gastrointestinal cancer, a lymphoma, a colon or colorectal cancer, an endometrial cancer, a thyroid cancer, or a bladder cancer.

Embodiment 329 is the method, use, or linker polypeptide for use of any one of embodiments 322-328, wherein the cancer is a microsatellite instability-high cancer.

Embodiment 330 is the method, use, or linker polypeptide for use of any one of embodiments 322-329, wherein the cancer is mismatch repair deficient.

Embodiment 331 is a nucleic acid encoding the linker polypeptide of any one of embodiments 1-319.

Embodiment 332 is an expression vector comprising the nucleic acid of the immediately preceding embodiment.

Embodiment 333 is a host cell comprising the nucleic acid of embodiment 331 or the vector of embodiment 332.

Embodiment 334 is a method of producing a linker polypeptide, comprising culturing the host cell of the immediately preceding embodiment under conditions wherein the linker polypeptide is produced.

Embodiment 335 is the method of the immediately preceding embodiment, further comprising isolating the linker polypeptide.

### FIGURE LEGENDS

FIG. 1A shows an illustration of a structure of an exemplary linker polypeptide and an SDS-PAGE gel (with Coomassie stain) characterizing multiple purified linker polypeptides.

FIGS. 1B-1C each shows SDS-PAGE gels (with Coomassie stain) characterizing multiple purified linker polypeptides.

FIG. 1D shows an illustration of another exemplary linker polypeptide structure and an SDS-PAGE gel (with Coomassie stain) characterizing multiple purified linker polypeptides.

FIGS. 2A-2F each show one or more SDS-PAGE gels followed by immunoblotting characterizing multiple linker polypeptides, with and without treatment with matrix metallopeptidase 9 (MMP9).

FIGS. 3A-3BB each show the results of an HEK Blue IL-2 assay that measured IL-2 and IL-15 activity of a specific linker polypeptide, with and without treatment with an MMP.

FIG. 4A shows an illustration of structures of different MMP linker peptides in linker polypeptides, in particular linker peptides that bind heparin.

FIG. 4B shows the results of assays that measured binding of the linker peptides of FIG. 4A to heparin.

FIG. 4C shows an illustration of structures of different MMP linker peptides in linker polypeptides, in particular linker peptides that bind fibronectin, and also shows the results of assays that measured binding of the linker peptides to fibronectin.

FIG. 4D shows an illustration of structures of different MMP linker peptides in linker polypeptides, in particular linker peptides that bind collagen, and also shows the results of assays that measured binding of the linker peptides to collagen.

FIG. 4E shows an illustration of structures of different linker polypeptides, and also shows the results of assays that measured binding to heparin by the linker polypeptides.

FIG. 4F shows the results of assays that measured binding to heparin by different linker polypeptides, including those that share the same heparin binding motif as the linker polypeptide Construct CC in FIG. 4E. The asterisk (*) denotes that for Construct NN, software was unable to compute the EC₅₀ based on fit; however, the Construct NN binding curve mimicked the Construct CC binding profile.

FIG. 4G shows the results of assays that measured binding to heparin by different linker polypeptides, including those that share the same heparin binding motif as the linker polypeptide Construct CC in FIG. 4E.

FIG. 4H shows the results of assays that measured binding to heparin by different linker polypeptides, including those that share the same heparin binding motif as the linker polypeptide Construct Y in FIG. 4E.

FIG. 4I shows the results of assays that measured binding to heparin by different linker polypeptides, including those that share the same heparin binding motif as the linker polypeptide Construct Y in FIG. 4E.

FIG. 4J shows the results of assays that measured binding to heparin by different IL-15Ra-IL-15 linker polypeptides.

FIG. 4K shows the results of assays that measured binding to fibronectin by different linker polypeptides.

FIG. 4L shows the results of a pulldown assay that measured binding to collagen by different linker polypeptides.

FIG. 4M shows the results of assays that measured binding to heparin by different linker polypeptides, with or without heparin binding sites.

FIG. 5A shows the results of real-time whole-body imaging for measuring in vivo levels of IL-2 fusion proteins in tumors, using fluorescently labelled proteins. FIG. 5B shows the levels of fusion proteins in FIG. 5A.

FIG. 6 shows the measurements of tumor volumes in C57BL/6 mice inoculated with B16F10 melanoma cells and treated with different linker polypeptides, and also shows a schematic drawing ranking the anti-tumor activity of the different linker polypeptides.

FIGS. 7A-7D respectively show the results of assays measuring levels of full-length fusion proteins in tumors (FIG. 7A), levels of IL-2 in tumors (FIG. 7B), levels of IFN-γ in tumors (FIG. 7C), and levels of full-length fusion proteins in serum (FIG. 7D).

FIGS. 8A-8B respectively show the results of assays measuring serum levels of TNF-α (FIG. 8A) and IL-6 (FIG. 8B) after animals were treated with different linker polypeptides.

FIG. 8C shows the results of an AST activity assay after animals were treated with different linker polypeptides.

FIGS. 9A-9D each illustrate a linker polypeptide according to certain embodiments of the disclosure. (AD, active domain; PM, pharmacokinetic modulator; CL, protease-cleavable polypeptide sequence and optionally a targeting sequence; IBD, immunoglobulin antigen-binding domain; D, chemotherapy drug.)

FIGS. 10A-10B each illustrate a linker polypeptide according to certain embodiments of the disclosure. (AD, active domain; PM, pharmacokinetic modulator; CL, protease-cleavable polypeptide sequence and optionally a targeting sequence; IBD, immunoglobulin antigen-binding domain; RBD, receptor-binding domain; CY, cytokine polypeptide sequence.)

FIGS. 11A-11B each illustrate release of the first active domain from the remainder of the linker polypeptide after the one or more protease-cleavable polypeptide sequences are cleaved. (AD, active domain; PM, pharmacokinetic modulator; CL, protease-cleavable polypeptide sequence and optionally a targeting sequence; IBD, immunoglobulin antigen-binding domain; D, chemotherapy drug.)

FIGS. 12A-12B each illustrate release of the first active domain from the remainder of the linker polypeptide after the one or more protease-cleavable polypeptide sequences are cleaved. (AD, active domain; PM, pharmacokinetic modulator; CL, protease-cleavable polypeptide sequence and optionally a targeting sequence; IBD, immunoglobulin antigen-binding domain; RBD, receptor-binding domain; CY, cytokine polypeptide sequence.)

FIGS. 13A-13C show the effects on tumor xenografts by treatment of different fusion proteins. Mean tumor volume is shown in FIGS. 13A-13B, and inhibition of tumor volume is shown in FIG. 13C.

FIG. 13D shows levels of IFN-7 in mice having tumor xenografts and treated with different fusion proteins.

FIGS. 14A-14E show results from flow cytometric analyses for select immune cell populations within harvested tumors in a mouse syngeneic model.

FIG. 15A shows schematics of asymmetrical IL-2 Fc fusion proteins containing ECM targeting sequences and single or dual masks.

FIG. 15B shows results of an SDS-PAGE analysis of asymmetrical IL-2 Fc fusion proteins.

FIGS. 15C-15U each show the results of an HEK Blue IL-2 assay that measured IL-2 activity of a specific asymmetrical IL-2 Fc fusion protein, with and without treatment with an MMP.

FIGS. 15V-15X show results from assays that measured binding to heparin and fibronectin by different asymmetrical IL-2 Fc fusion proteins, with or without heparin or fibronectin binding sites.

FIG. 15Y shows results from assays that measured binding to collagen by different asymmetrical IL-2 Fc fusion proteins, with or without a collagen binding site.

### DETAILED DESCRIPTION OF CERTAIN EMBODIMENTS

This specification describes exemplary embodiments and applications of the disclosure. The disclosure, however, is not limited to these exemplary embodiments and applications or to the manner in which the exemplary embodiments and applications operate or are described herein. The term “or” is used in an inclusive sense, i.e., equivalent to “and/or,” unless the context dictates otherwise. It is noted that, as used in this specification and the appended claims, the singular forms “a,” “an,” and “the,” and any singular use of any word, include plural referents unless expressly and unequivocally limited to one referent. As used herein, the terms “comprise,” “include,” and grammatical variants thereof are intended to be non-limiting, such that recitation of items in a list is not to the exclusion of other like items that can be substituted or added to the listed items. Section divisions in the specification are provided for the convenience of the reader only and do not limit any combination of elements discussed. In case of any contradiction or conflict between material incorporated by reference and the expressly described content provided herein, the expressly described content controls.

### Overview

Provided herein are linker polypeptides comprising a first targeting sequence; a second targeting sequence; and a first linker between the first targeting sequence and the second targeting sequence, the linker comprising a protease-cleavable polypeptide sequence. In some embodiments, the linker polypeptide comprises a first active domain; a second active domain; a pharmacokinetic modulator; and a first linker between the pharmacokinetic modulator and the first active domain, the first linker comprising a protease-cleavable polypeptide sequence. In some embodiments, the linker polypeptide comprises a first active domain; an inhibitory polypeptide sequence capable of blocking an activity of the first active domain; a first linker between the first active domain and the inhibitory polypeptide sequence, the linker comprising a protease-cleavable polypeptide sequence; and a first targeting sequence.

Proteolysis of the protease-cleavable polypeptide sequence can release the first and/or second binding domain, so that it can, for example, neutralize a tumor antigen and/or activate immune cells. Additionally, in some embodiments, each of the active domains can bind growth factor to reduce the extent to which the growth factor exerts an activity in vivo, such as stimulating cancer cell growth.

In some embodiments, the protease-cleavable polypeptide sequence is cleavable by a protease expressed at higher levels in the tumor microenvironment (TME) than in healthy tissue of the same type. In some embodiments, the protease-cleavable polypeptide sequence is a matrix metalloprotease (MMP)-cleavable linker, such as any of the MMP-cleavable linkers described herein. Without wishing to be bound by any particular theory, increased expression and/or activation of proteases, including but not necessarily limited to MMPs, in the tumor microenvironment (TME) can provide a mechanism for achieving selective or preferential activation of the linker polypeptide at or near a tumor site. Certain protease-cleavable polypeptide sequences described herein are considered particularly suitable for achieving such selective or preferential activation.

In other embodiments, the first and/or second targeting sequence binds an extracellular matrix component, an integrin, or a syndecan, or is configured to bind fibronectin in a pH-sensitive manner. In some embodiments, the targeting sequence is a targeting sequence described herein, e.g., a targeting sequence configured to bind an extracellular matrix component, heparin, an integrin, or a syndecan; or configured to bind an extracellular matrix component, heparin, IgB (CD79b), an integrin, a cadherin, a heparan sulfate proteoglycan, a syndecan, or a fibronectin in a pH-sensitive manner; or a targeting sequence comprising the sequence of any one of SEQ ID NOs: 179-665. The targeting sequence can facilitate accumulation and/or increased residence time of the linker polypeptide and/or the released active domain in the extracellular matrix (ECM). In some embodiments, a targeting sequence is combined with a protease-cleavable polypeptide sequence expressed at higher levels in the TME and/or cleavable by an MMP.

In some embodiments, the pharmacokinetic modulator may, for example, extend the half-life of the linker polypeptide.

Sequences of exemplary components of linker polypeptides are shown in Tables 1 and 2. In Table 1, “X_{Hy}” designates a hydrophobic amino acid residue. In some embodiments, the hydrophobic amino acid residue is any one of glycine (Gly), alanine (Ala), valine (Val), leucine (Leu), isoleucine (Ile), proline (Pro), phenylalanine (Phe), methionine (Met), and tryptophan (Trp). In some embodiments, the hydrophobic amino acid residue is any one of Ala, Leu, Val, Ile, Pro, Phe, Met, and Trp. In some embodiments, the hydrophobic amino acid residue is any one of Leu, Val, Ile, Pro, Phe, Met, and Trp. In some embodiments, the hydrophobic amino acid residue is any one of Ala, Leu, Val, Ile, Phe, Met, and Trp. In some embodiments, the hydrophobic amino acid residue is any one of Leu, Val, Ile, Phe, Met, and Trp. “(Pip)” represents piperidine. “(Hof)” represents homophenylalanine. “(Cit)” represents citrulline. “(Et)” represents ethionine. “C(me)” represents methylcysteine. In certain sequences, underlining is used to indicate mutated positions.

This disclosure further provides uses of these linker polypeptides, e.g., for treating cancer. In some embodiments, the linker polypeptide is selectively or preferentially cleaved in the tumor microenvironment, which may result in beneficial effects, e.g., improved recruitment and/or activation of immune cells in the vicinity of the tumor, and/or reduced systemic exposure to certain components of the linker polypeptides.

TABLE 1
Table of Sequences of Linker Polypeptides and Components Thereof

| | | | | | |
|---|---|---|---|---|---|
| SEQ | | | | | |
| ID | | | | | |
| NO | Description | Sequence | Species | Function | Notes |

IL-2 sequences

| | | | | | |
|---|---|---|---|---|---|
| 1 | h IL-2 | APTSSSTKKTQLQLEHLLLDLQMILNGINNYKNPKLTRMLTFKFYMPKKATE | human | cytokine | wild-type |
| | | LKHLQCLEEELKPLEEVLNLAQSKNFHLRPRDLISNINVIVLELKGSETTFM | | | |
| | | CEYADETATIVEFLNRWITFCQSIISTLT | | | |
| 2 | h IL-2 | APTSSSTKKTQLQLEHLLLDLQMILNGINNYKNPKLTRMLTFKFYMPKKATE | human | cytokine | C125 to S |
| | (C125S) | LKHLQCLEEELKPLEEVLNLAQSKNFHLRPRDLISNINVIVLELKGSETTFM | | | mutation |
| | | CEYADETATIVEFLNRWITFSQSIISTLT | | | |
| 3 | m IL-2 | APTSSSTSSSTAEAQQQQQQQQQQQQHLEQLLMDLQELLSRMENYRNLKLPR | mouse | cytokine | wild-type |
| | | MLTFKFYLPKQATELKDLQCLEDELGPLRHVLDLTQSKSFQLEDAENFISNI | | | |
| | | RVTVVKLKGSDNTFECQFDDESATVVDFLRRWIAFCQSIISTSPQ | | | |
| 4 | m IL-2 | APTSSSTSSSTAEAQQQQQQQQQQQQHLEQLLMDLQELLSRMENYRNLKLPR | mouse | cytokine | C140 to S |
| | (C140S) | MLTFKFYLPKQATELKDLQCLEDELGPLRHVLDLTQSKSFQLEDAENFISNI | | | mutation |
| | | RVTVVKLKGSDNTFECQFDDESATVVDFLRRWIAFSQSIISTSPQ | | | |
| 5-9 | Not Used | | | | |

IL-10 sequences

| | | | | | |
|---|---|---|---|---|---|
| 900 | IL-10 | SPGQGTQSENSCTHFPGNLPNMLRDLRDAFSRVKTFFQMKDQLDNLLLKESL | human | cytokine | wild-type |
| | | LEDFKGYLGCQALSEMIQFYLEEVMPQAENQDPDIKAHVNSLGENLKTLRLR | | | |
| | | LRRCHRFLPCENKSKAVEQVKNAFNKLQEKGIYKAMSEFDIFINYIEAYMTM | | | |
| | | KIRN | | | |

IL-15 sequences

| | | | | | |
|---|---|---|---|---|---|
| 901 | IL-15 | NWVNVISDLKKIEDLIQSMHIDATLYTESDVHPSCKVTAMKCFLLELQVISL | human | cytokine | wild-type |
| | | ESGDASIHDTVENLIILANNSLSSNGNVTESGCKECEELEEKNIKEFLQSFV | | | |
| | | HIVQMFINTS | | | |

CXCL9 sequences

| | | | | | |
|---|---|---|---|---|---|
| 902 | CXCL9 | TPVVRKGRCSCISTNQGTIHLQSLKDLKQFAPSPSCEKIEIIATLKNGVQTC | human | chemokine | wild-type |
| | | LNPDSADVKELIKKWEKQVSQKKKQKNGKKHQKKKVLKVRKSQRSRQKKTT | | | |

CXCL10 sequences

| | | | | | |
|---|---|---|---|---|---|
| 903 | CXCL10 | VPLSRTVRCTCISISNQPVNPRSLEKLEIIPASQFCPRVEIIATMKKKGEKR | human | chemokine | wild-type |
| | | CLNPESKAIKNLLKAVSKERSKRSP | | | |

TGF-β sequences

| | | | | | |
|---|---|---|---|---|---|
| 904 | hu TGFb 1 | LSTCKTIDMELVKRKRIEAIRGQILSKLRLASPPSQGEVPPGPLPEAVLALY | human | cytokine | wild-type |
| | | NSTRDRVAGESAEPEPEPEADYYAKEVTRVLMVETHNEIYDKFKQSTHSIYM | | | ligand |
| | | FFNTSELREAVPEPVLLSRAELRLLRLKLKVEQHVELYQKYSNNSWRYLSNR | | | |
| | | LLAPSDSPEWLSFDVTGVVRQWLSRGGEIEGFRLSAHCSCDSRDNTLQVDIN | | | |
| | | GFTTGRRGDLATIHGMNRPFLLLMATPLERAQHLQSSRHRRALDTNYCFSST | | | |
| | | EKNCCVRQLYIDFRKDLGWKWIHEPKGYHANFCLGPCPYIWSLDTQYSKVLA | | | |
| | | LYNQHNPGASAAPCCVPQALEPLPIVYYVGRKPKVEQLSNMIVRSCKCS | | | |
| 905 | hu TGFb 2 | LSTCSTLDMDQFMRKRIEAIRGQILSKLKLTSPPEDYPEPEEVPPEVISIYN | human | cytokine | wild-type |
| | | STRDLLQEKASRRAAACERERSDEEYYAKEVYKIDMPPFFPSENAIPPTFYR | | | ligand |
| | | PYFRIVRFDVSAMEKNASNLVKAEFRVFRLQNPKARVPEQRIELYQILKSKD | | | |
| | | LTSPTQRYIDSKVVKTRAEGEWLSFDVTDAVHEWLHHKDRNLGFKISLHCPC | | | |
| | | CTFVPSNNYIIPNKSEELEARFAGIDGTSTYTSGDQKTIKSTRKKNSGKTPH | | | |
| | | LLLMLLPSYRLESQQTNRRKKRALDAAYCFRNVQDNCCLRPLYIDFKRDLGW | | | |
| | | KWIHEPKGYNANFCAGACPYLWSSDTQHSRVLSLYNTINPEASASPCCVSQD | | | |
| | | LEPLTILYYIGKTPKIEQLSNMIVKSCKCS | | | |
| 906 | hu TGFb 3 | LSTCTTLDFGHIKKKRVEAIRGQILSKLRLTSPPEPTVMTHVPYQVLALYNS | human | cytokine | wild-type |
| | | TRELLEEMHGEREEGCTQENTESEYYAKEIHKFDMIQGLAEHNELAVCPKGI | | | ligand |
| | | TSKVFRFNVSSVEKNRTNLFRAEFRVLRVPNPSSKRNEQRIELFQILRPDEH | | | |
| | | IAKQRYIGGKNLPTRGTAEWLSFDVTDTVREWLLRRESNLGLEISIHCPCHT | | | |
| | | FQPNGDILENIHEVMEIKFKGVDNEDDHGRGDLGRLKKQKDHHNPHLILMMI | | | |
| | | PPHRLDNPGQGGQRKKRALDTNYCFRNLEENCCVRPLYIDFRQDLGWKWVHE | | | |
| | | PKGYYANFCSGPCPYLRSADTTHSTVLGLYNTLNPEASASPCCVPQDLEPLT | | | |
| | | ILYYVGRTPKVEQLSNMVVKSCKCS | | | |

Immunoglobulin sequences

| | | | | | |
|---|---|---|---|---|---|
| 907 | Trastuzumab | DIQMTQSPSSLSASVGDRVTITCRASQDVNTAVAWYQQKPGKAPKLLIYSAS | human- | bio- | anti-Her2 |
| | light chain | FLYSGVPSRFSGSRSGTDFTLTISSLQPEDFATYYCQQHYTTPPTFGQGTKV | ized | logic | |
| | | EIKRTVAAPSVFIFPPSDEQLKSGTASVVCLLNNFYPREAKVQWKVDNALQS | | | |
| | | GNSQESVTEQDSKDSTYSLSSTLTLSKADYEKHKVYACEVTHQGLSSPVTKS | | | |
| | | FNRGEC | | | |
| 908 | Trastuzumab | EVQLVESGGGLVQPGGSLRLSCAASGFNIKDTYIHWVRQAPGKGLEWVARIY | human- | bio- | anti-Her2 |
| | heavy chain | PTNGYTRYADSVKGRFTISADTSKNTAYLQMNSLRAEDTAVYYCSRWGGDGF | ized | logic | |
| | | YAMDYWGQGTLVTVSSASTKGPSVFPLAPSSKSTSGGTAALGCLVKDYFPEP | | | |
| | | VTVSWNSGALTSGVHTFPAVLQSSGLYSLSSVVTVPSSSLGTQTYICNVNHK | | | |
| | | PSNTKVDKKVEPKSCDKTHTCPPCPAPELLGGPSVFLFPPKPKDTLMISRTP | | | |
| | | EVTCVVVDVSHEDPEVKFNWYVDGVEVHNAKTKPREEQYNSTYRVVSVLTVL | | | |
| | | HQDWLNGKEYKCKVSNKALPAPIEKTISKAKGQPREPQVYTLPPSREEMTKN | | | |
| | | QVSLTCLVKGFYPSDIAVEWESNGQPENNYKTTPPVLDSDGSFFLYSKLTVD | | | |
| | | KSRWQQGNVFSCSVMHEALHNHYTQKSLSLSPGK | | | |
| 909 | Trastuzumab | DIQMTQSPSSLSASVGDRVTITCRASQDVNTAVAWYQQKPGKAPKLLIYSAS | human- | bio- | anti-Her2 |
| | VL | FLYSGVPSRFSGSRSGTDFTLTISSLQPEDFATYYCQQHYTTPPTFGQGTKV | ized | logic | |
| | | EIK | | | |
| 910 | Trastuzumab | EVQLVESGGGLVQPGGSLRLSCAASGFNIKDTYIHWVRQAPGKGLEWVARIY | human- | bio- | anti-Her2 |
| | VH | PTNGYTRYADSVKGRFTISADTSKNTAYLQMNSLRAEDTAVYYCSRWGGDGF | ized | logic | |
| | | YAMDYWGQGTLVTVSS | | | |
| 911 | Cetuximab | DILLTQSPVILSVSPGERVSFSCRASQSIGTNIHWYQQRTNGSPRLLIKYAS | chimeric | bio- | anti-EGFR |
| | light chain | ESISGIPSRFSGSGSGTDFTLSINSVESEDIADYYCQQNNNWPTTFGAGTKL | (mouse/ | logic | |
| | | ELKRTVAAPSVFIFPPSDEQLKSGTASVVCLLNNFYPREAKVQWKVDNALQS | human) | | |
| | | GNSQESVTEQDSKDSTYSLSSTLTLSKADYEKHKVYACEVTHQGLSSPVTKS | | | |
| | | FNRGEC | | | |
| 912 | Cetuximab | QVQLKQSGPGLVQPSQSLSITCTVSGFSLTNYGVHWVRQSPGKGLEWLGVIW | chimeric | bio- | anti-EGFR |
| | heavy chain | SGGNTDYNTPFTSRLSINKDNSKSQVFFKMNSLQSNDTAIYYCARALTYYDY | (mouse/ | logic | |
| | | EFAYWGQGTLVTVSAASTKGPSVFPLAPSSKSTSGGTAALGCLVKDYFPEPV | human) | | |
| | | TVSWNSGALTSGVHTFPAVLQSSGLYSLSSVVTVPSSSLGTQTYICNVNHKP | | | |
| | | SNTKVDKKVEPKSCDKTHTCPPCPAPELLGGPSVFLFPPKPKDTLMISRTPE | | | |
| | | VTCVVVDVSHEDPEVKFNWYVDGVEVHNAKTKPREEQYNSTYRVVSVLTVLH | | | |
| | | QDWLNGKEYKCKVSNKALPAPIEKTISKAKGQPREPQVYTLPPSRDELTKNQ | | | |
| | | VSLTCLVKGFYPSDIAVEWESNGQPENNYKTTPPVLDSDGSFFLYSKLTVDK | | | |
| | | SRWQQGNVFSCSVMHEALHNHYTQKSLSLSPGK | | | |
| 913 | Cetuximab | DILLTQSPVILSVSPGERVSFSCRASQSIGTNIHWYQQRINGSPRLLIKYAS | chimeric | bio- | anti-EGFR |
| | VL | ESISGIPSRFSGSGSGTDFILSINSVESEDIADYYCQQNNNWPTTFGAGTKL | (mouse/ | logic | |
| | | ELK | human) | | |
| 914 | Cetuximab | QVQLKQSGPGLVQPSQSLSITCTVSGFSLTNYGVHWVRQSPGKGLEWLGVIW | chimeric | bio- | anti-EGFR |
| | VH | SGGNTDYNTPFTSRLSINKDNSKSQVFFKMNSLQSNDTAIYYCARALTYYDY | (mouse/ | logic | |
| | | EFAYWGQGTLVTVS | human) | | |
| 915 | Nivolumab | QVQLVESGGGVVQPGRSLRLDCKASGITFSNSGMHWVRQAPGKGLEWVAVIW | human | bio- | anti-PD-1 |
| | heavy chain | YDGSKRYYADSVKGRFTISRDNSKNTLFLQMNSLRAEDTAVYYCATNDDYWG | | logic | |
| | | QGTLVTVSSASTKGPSVFPLAPCSRSTSESTAALGCLVKDYFPEPVTVSWNS | | | |
| | | GALTSGVHTFPAVLQSSGLYSLSSVVTVPSSSLGTKTYTCNVDHKPSNTKVD | | | |
| | | KRVESKYGPPCPPCPAPEFLGGPSVFLFPPKPKDTLMISRTPEVTCVVVDVS | | | |
| | | QEDPEVQFNWYVDGVEVHNAKTKPREEQFNSTYRVVSVLTVLHQDWLNGKEY | | | |
| | | KCKVSNKGLPSSIEKTISKAKGQPREPQVYTLPPSQEEMTKNQVSLTCLVKG | | | |
| | | FYPSDIAVEWESNGQPENNYKTTPPVLDSDGSFFLYSRLTVDKSRWQEGNVF | | | |
| | | SCSVMHEALHNHYTQKSLSLSLGK | | | |
| 916 | Nivolumab | EIVLTQSPAILSLSPGERATLSCRASQSVSSYLAWYQQKPGQAPRLLIYDAS | human | bio- | anti-PD-1 |
| | light chain | NRATGIPARFSGSGSGTDFTLTISSLEPEDFAVYYCQQSSNWPRTFGQGTKV | | logic | |
| | | EIKRTVAAPSVFIFPPSDEQLKSGTASVVCLLNNFYPREAKVQWKVDNALQS | | | |
| | | GNSQESVTEQDSKDSTYSLSSTLTLSKADYEKHKVYACEVTHQGLSSPVIKS | | | |
| | | FNRGEC | | | |
| 917 | Nivolumab | QVQLVESGGGVVQPGRSLRLDCKASGITFSNSGMHWVRQAPGKGLEWVAVIW | human | bio- | anti-PD-1 |
| | VH | YDGSKRYYADSVKGRFTISRDNSKNTLFLQMNSLRAEDTAVYYCAINDDYWG | | logic | |
| | | QGTLVTVSS | | | |
| 918 | Nivolumab | EIVLIQSPATLSLSPGERATLSCRASQSVSSYLAWYQQKPGQAPRLLIYDAS | human | bio- | anti-PD-1 |
| | VL | NRATGIPARFSGSGSGTDFTLTISSLEPEDFAVYYCQQSSNWPRTFGQGTKV | | logic | |
| | | EIK | | | |
| 919 | atezolizumab | EVQLVESGGGLVQPGGSLRLSCAASGFTFSDSWIHWVRQAPGKGLEWVAWIS | human- | bio- | anti-PD-L1 |
| | heavy chain | PYGGSTYYADSVKGRFTISADTSKNTAYLQMNSLRAEDTAVYYCARRHWPGG | ized | logic | |
| | | FDYWGQGTLVTVSSASTKGPSVFPLAPSSKSTSGGTAALGCLVKDYFPEPVT | | | |
| | | VSWNSGALISGVHTFPAVLQSSGLYSLSSVVTVPSSSLGTQTYICNVNHKPS | | | |
| | | NTKVDKKVEPKSCDKTHTCPPCPAPELLGGPSVFLFPPKPKDTLMISRTPEV | | | |
| | | TCVVVDVSHEDPEVKFNWYVDGVEVHNAKTKPREEQYASTYRVVSVLTVLHQ | | | |
| | | DWLNGKEYKCKVSNKALPAPIEKTISKAKGQPREPQVYTLPPSREEMTKNQV | | | |
| | | SLTCLVKGFYPSDIAVEWESNGQPENNYKTTPPVLDSDGSFFLYSKLTVDKS | | | |
| | | RWQQGNVFSCSVMHEALHNHYTQKSLSLSPGK | | | |
| 920 | atezolizumab | DIQMTQSPSSLSASVGDRVTITCRASQDVSTAVAWYQQKPGKAPKLLIYSAS | human- | bio- | anti-PD-L1 |
| | light chain | FLYSGVPSRFSGSGSGTDFTLTISSLQPEDFATYYCQQYLYHPATFGQGTKV | ized | logic | |
| | | EIKRTVAAPSVFIFPPSDEQLKSGTASVVCLLNNFYPREAKVQWKVDNALQS | | | |
| | | GNSQESVTEQDSKDSTYSLSSTLTLSKADYEKHKVYACEVTHQGLSSPVTKS | | | |
| | | FNRGEC | | | |
| 921 | atezolizumab | EVQLVESGGGLVQPGGSLRLSCAASGFTFSDSWIHWVRQAPGKGLEWVAWIS | human- | bio- | anti-PD-L1 |
| | VH | PYGGSTYYADSVKGRFTISADTSKNTAYLQMNSLRAEDTAVYYCARRHWPGG | ized | logic | |
| | | FDYWGQGTLVTVSS | | | |
| 922 | atezolizumab | DIQMTQSPSSLSASVGDRVTITCRASQDVSTAVAWYQQKPGKAPKLLIYSAS | human- | bio- | anti-PD-L1 |
| | VL | FLYSGVPSRFSGSGSGTDFTLTISSLQPEDFATYYCQQYLYHPATFGQGTKV | ized | logic | |
| | | EIK | | | |
| 923 | Teplizumab | QVQLVQSGGGVVQPGRSLRLSCKASGYTFTRYTMHWVRQAPGKGLEWIGYIN | human- | bio- | anti-CD3 |
| | heavy chain | PSRGYTNYNQKVKDRFTISRDNSKNTAFLQMDSLRPEDTGVYFCARYYDDHY | ized | logic | |
| | | CLDYWGQGTPVTVSSASTKGPSVFPLAPSSKSTSGGTAALGCLVKDYFPEPV | | | |
| | | TVSWNSGALTSGVHTFPAVLQSSGLYSLSSVVTVPSSSLGTQTYICNVNHKP | | | |
| | | SNTKVDKKVEPKSCDKTHTCPPCPAPEAAGGPSVFLFPPKPKDTLMISRTPE | | | |
| | | VTCVVVDVSHEDPEVKFNWYVDGVEVHNAKTKPREEQYNSTYRVVSVLTVLH | | | |
| | | QDWLNGKEYKCKVSNKALPAPIEKTISKAKGQPREPQVYTLPPSRDELTKNQ | | | |
| | | VSLTCLVKGFYPSDIAVEWESNGQPENNYKTTPPVLDSDGSFFLYSKLTVDK | | | |
| | | SRWQQGNVFSCSVMHEALHNHYTQKSLSLSPGK | | | |
| 924 | Teplizumab | DIQMTQSPSSLSASVGDRVTITCSASSSVSYMNWYQQTPGKAPKRWIYDTSK | human- | bio- | anti-CD3 |
| | light chain | LASGVPSRFSGSGSGTDYTFTISSLQPEDIATYYCQQWSSNPFTFGQGTKLQ | ized | logic | |
| | | ITRTVAAPSVFIFPPSDEQLKSGTASVVCLLNNFYPREAKVQWKVDNALQSG | | | |
| | | NSQESVTEQDSKDSTYSLSSTLTLSKADYEKHKVYACEVTHQGLSSPVTKSF | | | |
| | | NRGEC | | | |
| 925 | Teplizumab | QVQLVQSGGGVVQPGRSLRLSCKASGYTFTRYTMHWVRQAPGKGLEWIGYIN | human- | bio- | anti-CD3 |
| | VH | PSRGYTNYNQKVKDRFTISRDNSKNTAFLQMDSLRPEDTGVYFCARYYDDHY | ized | logic | |
| | | CLDYWGQGTPVTVSS | | | |
| 926 | Teplizumab | DIQMTQSPSSLSASVGDRVTITCSASSSVSYMNWYQQTPGKAPKRWIYDTSK | human- | bio- | anti-CD3 |
| | VL | LASGVPSRFSGSGSGTDYTFTISSLQPEDIATYYCQQWSSNPFTFGQGTKLQ | ized | logic | |
| | | IT | | | |
| 927 | muromonab | QVQLQQSGAELARPGASVKMSCKASGYTFTRYTMHWVKQRPGQGLEWIGYIN | mouse | bio- | anti-CD3 |
| | heavy chain | PSRGYTNYNQKFKDKATLTTDKSSSTAYMQLSSLTSEDSAVYYCARYYDDHY | | logic | |
| | | CLDYWGQGTTLTVSSAKTTAPSVYPLAPVCGGTTGSSVTLGCLVKGYFPEPV | | | |
| | | TLTWNSGSLSSGVHTFPAVLQSDLYTLSSSVTVTSSTWPSQSITCNVAHPAS | | | |
| | | STKVDKKIEPRPKSCDKTHTCPPCPAPELLGGPSVFLFPPKPKDTLMISRTP | | | |
| | | EVTCVVVDVSHEDPEVKFNWYVDGVEVHNAKTKPREEQYNSTYRVVSVLTVL | | | |
| | | HQDWLNGKEYKCKVSNKALPAPIEKTISKAKGQPREPQVYTLPPSRDELTKN | | | |
| | | QVSLTCLVKGFYPSDIAVEWESNGQPENNYKTTPPVLDSDGSFFLYSKLTVD | | | |
| | | KSRWQQGNVFSCSVMHEALHNHYTQKSLSLSPGK | | | |
| 928 | muromonab | QIVLTQSPAIMSASPGEKVTMTCSASSSVSYMNWYQQKSGTSPKRWIYDTSK | mouse | bio- | anti-CD3 |
| | light chain | LASGVPAHFRGSGSGTSYSLTISGMEAEDAATYYCQQWSSNPFTFGSGTKLE | | logic | |
| | | INRADTAPTVSIFPPSSEQLTSGGASVVCFLNNFYPKDINVKWKIDGSERQN | | | |
| | | GVLNSWTDQDSKDSTYSMSSTLTLTKDEYERHNSYTCEATHKTSTSPIVKSF | | | |
| | | NRNEC | | | |
| 929 | muromonab | QVQLQQSGAELARPGASVKMSCKASGYTFTRYTMHWVKQRPGQGLEWIGYIN | mouse | bio- | anti-CD3 |
| | VH | PSRGYTNYNQKFKDKATLTTDKSSSTAYMQLSSLTSEDSAVYYCARYYDDHY | | logic | |
| | | CLDYWGQGTTLTVSS | | | |
| 930 | muromonab | QIVLTQSPAIMSASPGEKVTMTCSASSSVSYMNWYQQKSGTSPKRWIYDTSK | mouse | bio- | anti-CD3 |
| | VL | LASGVPAHFRGSGSGTSYSLTISGMEAEDAATYYCQQWSSNPFTFGSGTKLE | | logic | |
| | | IN | | | |
| 931 | otelixizumab | EVQLLESGGGLVQPGGSLRLSCAASGFTFSSFPMAWVRQAPGKGLEWVSTIS | chimeric | bio- | anti-CD3 |
| | heavy chain | TSGGRTYYRDSVKGRFTISRDNSKNTLYLQMNSLRAEDTAVYYCAKFRQYSG | (mouse/ | logic | |
| | | GFDYWGQGTLVTVSSASTKGPSVFPLAPSSKSTSGGTAALGCLVKDYFPEPV | human) | | |
| | | TVSWNSGALTSGVHTFPAVLQSSGLYSLSSVVTVPSSSLGTQTYICNVNHKP | | | |
| | | SNTKVDKKVEPKSCDKTHTCPPCPAPELLGGPSVFLFPPKPKDTLMISRTPE | | | |
| | | VTCVVVDVSHEDPEVKFNWYVDGVEVHNAKTKPREEQYASTYRVVSVLTVLH | | | |
| | | QDWLNGKEYKCKVSNKALPAPIEKTISKAKGQPREPQVYTLPPSRDELTKNQ | | | |
| | | VSLTCLVKGFYPSDIAVEWESNGQPENNYKTTPPVLDSDGSFFLYSKLTVDK | | | |
| | | SRWQQGNVFSCSVMHEALHNHYTQKSLSLSPGK | | | |
| 932 | otelixizumab | DIQLTQPNSVSTSLGSTVKLSCTLSSGNIENNYVHWYQLYEGRSPTTMIYDD | chimeric | bio- | anti-CD3 |
| | light chain | DKRPDGVPDRFSGSIDRSSNSAFLTIHNVAIEDEAIYFCHSYVSSFNVFGGG | (mouse/ | logic | |
| | | TKLTVLRQPKAAPSVTLFPPSSEELQANKATLVCLISDFYPGAVTVAWKADS | human) | | |
| | | SPVKAGVETTTPSKQSNNKYAASSYLSLTPEQWKSHRSYSCQVTHEGSTVEK | | | |
| | | TVAPTECS | | | |
| 933 | otelixizumab | EVQLLESGGGLVQPGGSLRLSCAASGFTFSSFPMAWVRQAPGKGLEWVSTIS | chimeric | bio- | anti-CD3 |
| | VH | TSGGRTYYRDSVKGRFTISRDNSKNTLYLQMNSLRAEDTAVYYCAKFRQYSG | (mouse/ | logic | |
| | | GFDYWGQGTLVTVSS | human) | | |
| 934 | otelixizumab | DIQLTQPNSVSTSLGSTVKLSCTLSSGNIENNYVHWYQLYEGRSPTTMIYDD | chimeric | bio- | anti-CD3 |
| | VL | DKRPDGVPDRFSGSIDRSSNSAFLTIHNVAIEDEAIYFCHSYVSSFNVFGGG | (mouse/ | logic | |
| | | TKLTVLR | human) | | |
| 935 | visilizumab | QVQLVQSGAEVKKPGASVKVSCKASGYTFISYTMHWVRQAPGQGLEWMGYIN | human- | bio- | anti-CD3 |
| | heavy chain | PRSGYTHYNQKLKDKATLTADKSASTAYMELSSLRSEDTAVYYCARSAYYDY | ized | logic | |
| | | DGFAYWGQGTLVTVSSASTKGPSVFPLAPCSRSTSESTAALGCLVKDYFPEP | | | |
| | | VTVSWNSGALTSGVHTFPAVLQSSGLYSLSSVVTVPSSNFGTQTYTCNVDHK | | | |
| | | PSNTKVDKTVERKCCVECPPCPAPPAAAPSVFLFPPKPKDTLMISRTPEVTC | | | |
| | | VVVDVSHEDPEVQFNWYVDGVEVHNAKTKPREEQFNSTFRVVSVLTVVHQDW | | | |
| | | LNGKEYKCKVSNKGLPAPIEKTISKTKGQPREPQVYTLPPSREEMTKNQVSL | | | |
| | | TCLVKGFYPSDIAVEWESNGQPENNYKTTPPMLDSDGSFFLYSKLTVDKSRW | | | |
| | | QQGNVFSCSVMHEALHNHYTQKSLSLSPSK | | | |
| 936 | visilizumab | DIQMTQSPSSLSASVGDRVTITCSASSSVSYMNWYQQKPGKAPKRLIYDTSK | human- | bio- | anti-CD3 |
| | light chain | LASGVPSRFSGSGSGTDFTLTISSLQPEDFATYYCQQWSSNPPTFGGGTKVE | ized | logic | |
| | | IKRTVAAPSVFIFPPSDEQLKSGTASVVCLLNNFYPREAKVQWKVDNALQSG | | | |
| | | NSQESVTEQDSKDSTYSLSSTLTLSKADYEKHKVYACEVTHQGLSSPVTKSF | | | |
| | | NRGEC | | | |
| 937 | visilizumab | QVQLVQSGAEVKKPGASVKVSCKASGYTFISYTMHWVRQAPGQGLEWMGYIN | human- | bio- | anti-CD3 |
| | VH | PRSGYTHYNQKLKDKATLTADKSASTAYMELSSLRSEDTAVYYCARSAYYDY | ized | logic | |
| | | DGFAYWGQGTLVTVSS | | | |
| 938 | visilizumab | DIQMTQSPSSLSASVGDRVTITCSASSSVSYMNWYQQKPGKAPKRLIYDTSK | human- | bio- | anti-CD3 |
| | VL | LASGVPSRFSGSGSGTDFTLTISSLQPEDFATYYCQQWSSNPPTFGGGTKVE | ized | logic | |
| | | IKR | | | |

Blockers: IL-2R sequences

| | | | | | |
|---|---|---|---|---|---|
| 10 | h IL-2Ralpha | ELCDDDPPEIPHATFKAMAYKEGTMLNCECKRGFRRIKSGSLYMLCTGNSSH | human | blocker | wild-type |
| | | SSWDNQCQCTSSATRNTTKQVTPQPEEQKERKTTEMQSPMQPVDQASLPGHC | | | amino acids |
| | | REPPPWENEATERIYHFVVGQMVYYQCVQGYRALHRGPAESVCKMTHGKTRW | | | 1-219 |
| | | TQPQLICTGEMETSQFPGEEKPQASPEGRPESETSCLVTTTDFQIQTEMAAT | | | |
| | | METSIFTTEYQ | | | |
| 11 | h IL-2Ralpha | ELCDDDPPEIPHATFKAMAYKEGTMLNCECKRGFRRIKSGSLYMLCTGNSSH | human | blocker | sushi domain |
| | (1-63) | SSWDNQCQCTS | | | 1 wild-type |
| 12 | h IL-2Ralpha | ELCDDDPPEIPHATFKAMAYKEGTILNCECKRGFRRIKSGSLYMLCTGNSSH | human | blocker | M25 to I |
| | (M25I) | SSWDNQCQCTSSATRNTTKQVTPQPEEQKERKTTEMQSPMQPVDQASLPGHC | | | mutation |
| | | REPPPWENEATERIYHFVVGQMVYYQCVQGYRALHRGPAESVCKMTHGKTRW | | | |
| | | TQPQLICTGEMETSQFPGEEKPQASPEGRPESETSCLVTTTDFQIQTEMAAT | | | |
| | | METSIFTTEYQ | | | |
| 13 | h IL-2Ralpha | ELCDDDPPEIPHATFKAMAYKEGTMLNCECKRGFRRIKSGSVYMLCTGNSSH | human | blocker | L42 to V |
| | (L42V) | SSWDNQCQCTSSATRNTTKQVTPQPEEQKERKTTEMQSPMQPVDQASLPGHC | | | mutation |
| | | REPPPWENEATERIYHFVVGQMVYYQCVQGYRALHRGPAESVCKMTHGKTRW | | | |
| | | TQPQLICTGEMETSQFPGEEKPQASPEGRPESETSCLVTTTDFQIQTEMAAT | | | |
| | | METSIFTTEYQ | | | |
| 14 | h IL-2Ralpha | ELCDDDPPEIPHATFKAMAYKEGTILNCECKRGFRRIKSGSVYMLCTGNSSH | human | blocker | M25 to I |
| | (M25I; L42V) | SSWDNQCQCTSSATRNTTKQVTPQPEEQKERKTTEMQSPMQPVDQASLPGHC | | | mutation; |
| | | REPPPWENEATERIYHFVVGQMVYYQCVQGYRALHRGPAESVCKMTHGKTRW | | | L42 to V |
| | | TQPQLICTGEMETSQFPGEEKPQASPEGRPESETSCLVTTTDFQIQTEMAAT | | | mutation |
| | | METSIFTTEYQ | | | |
| 15 | Human | LNTTILTPNGNEDTTADFFLTTMPTDSLSVSTLPLPEVQCFVFNVEYMNCTW | human | blocker | |
| | IL2Rgamma | NSSSEPQPTNLTLHYWYKNSDNDKVQKCSHYLFSEEITSGCQLQKKEIHLYQ | | | |
| | polypeptide | TFVVQLQDPREPRRQATQMLKLQNLVIPWAPENLTLHKLSESQLELNWNNRF | | | |
| | sequence | LNHCLEHLVQYRTDWDHSWTEQSVDYRHKFSLPSVDGQKRYTFRVRSRFNPL | | | |
| | | CGSAQHWSEWSHPIHWGSNTSKENPFLFALEA | | | |
| 16 | Human | AVNGTSQFTCFYNSRANISCVWSQDGALQDTSCQVHAWPDRRRWNQTCELLP | human | blocker | |
| | IL2Rbeta | VSQASWACNLILGAPDSQKLTTVDIVTLRVLCREGVRWRVMAIQDFKPFENL | | | |
| | polypeptide | RLMAPISLQVVHVETHRCNISWEISQASHYFERHLEFEARTLSPGHTWEEAP | | | |
| | sequence | LLTLKQKQEWICLETLTPDTQYEFQVRVKPLQGEFTTWSPWSQPLAFRTKPA | | | |
| | | ALGKDT | | | |
| 17 | chimeric IL- | ELCLYDPPEVPNATFKALSYKNGTILNCECKRGFRRLKELVYMRCLGNSWSS | human/ | blocker | mouse |
| | 2Ralpha | NCQCTSSATRNTTKQVTPQPEEQKERKTTEMQSPMQPVDQASLPGHCREPPP | mouse | | IL2Ralpha |
| | | WENEATERIYHFVVGQMVYYQCVQGYRALHRGPAESVCKMTHGKTRWTQPQL | | | (1-58)-hu |
| | | ICTGEMETSQFPGEEKPQASPEGRPESETSCLVTTTDFQIQTEMAATMETSI | | | IL2Ralpha |
| | | FTTEYQ | | | (64-219) |
| 18 | m IL-2Ralpha | ELCLYDPPEVPNATFKALSYKNGTILNCECKRGFRRLKELVYMRCLGNSWSS | mouse | blocker | wild-type |
| | | NCQCTSNSHDKSRKQVTAQLEHQKEQQTTTDMQKPTQSMHQENLTGHCREPP | | | amino acids |
| | | PWKHEDSKRIYHFVEGQSVHYECIPGYKALQRGPAISICKMKCGKTGWTQPQ | | | 1-215 |
| | | LTCVDEREHHRFLASEESQGSRNSSPESETSCPITTTDFPQPTETTAMTETF | | | |
| | | VLTMEYK | | | |
| 19 | m IL-2Ralpha | ELCLYDPPEVPNATFKALSYKNGTILNCECKRGFRRLKELVYMRCLGNSWSS | mouse | blocker | sushi domain |
| | (1-58) | NCQCTS | | | 1 wild-type |
| 20 | h IL-2Ralpha | ELCLYDPPEIPHATFKAMAYKEGTILNCECKRGFRRIKSGSLYMLCTGNSSH | human | blocker | D4 to L |
| | (1-219) | SSWDNQCQCTSSATRNTTKQVTPQPEEQKERKTTEMQSPMQPVDQASLPGHC | | | mutation; D5 |
| | | REPPPWENEATERIYHFVVGQMVYYQCVQGYRALHRGPAESVCKMTHGKTRW | | | to Y |
| | M25I/D4L/D5 | TQPQLICTGEMETSQFPGEEKPQASPEGRPESETSCLVTTTDFQ | | | mutation; |
| | Y | IQTEMAATMETSIFTTEYQ | | | M25 to I |
| | | | | | mutation |
| 21 | h IL-2Ralpha | ELCLYDPPEIPHATFKAMAYKEGTMLNCECKRGFRRIKSGSVYMLCTGNSSH | human | blocker | D4 to L |
| | (1-219) | SSWDNQCQCTSSATRNTTKQVTPQPEEQKERKTTEMQSPMQPVDQASLPGHC | | | mutation; D5 |
| | L42V/D4L/D | REPPPWENEATERIYHFVVGQMVYYQCVQGYRALHRGPAESVCKMTHGKTRW | | | to Y |
| | 5Y | TQPQLICTGEMETSQFPGEEKPQASPEGRPESETSCLVTTTDFQ | | | mutation; |
| | | IQTEMAATMETSIFTTEYQ | | | L42 to V |
| | | | | | mutation |
| 22 | h IL-2Ralpha | ELCLYDPPEIPHATFKAMAYKEGTILNCECKRGFRRIKSGSVYMLCTGNSSH | human | blocker | D4 to L |
| | (1-219) | SSWDNQCQCTSSATRNTTKQVTPQPEEQKERKTTEMQSPMQPVDQASLPGHC | | | mutation; D5 |
| | M25I/L42V/D | REPPPWENEATERIYHFVVGQMVYYQCVQGYRALHRGPAESVCKMTHGKTRW | | | to Y |
| | 4L/D5Y | TQPQLICTGEMETSQFPGEEKPQASPEGRPESETSCLVTTTDFQ | | | mutation; |
| | | IQTEMAATMETSIFTTEYQ | | | M25 to I |
| | | | | | mutation; |
| | | | | | L42 to V |
| | | | | | mutation |
| 23 | h IL-2Ralpha | ELCLYDPPEIPHATFKAMAYKEGTMLNCECKRGFRRIKSGSLYMLCTGNSSH | human | blocker | D4 to L |
| | (1- | SSWDNQCQCTSSATRNTTKQVTPQPEEQKERKTTEMQSPMQPVDQASLPGHC | | | mutation; D5 |
| | 219)D4L/D5 | REPPPWENEATERIYHFVVGQMVYYQCVQGYRALHRGPAESVCKMTHGKTRW | | | to Y |
| | Y | TQPQLICTGEMETSQFPGEEKPQASPEGRPESETSCLVTTTDFQ | | | mutation |
| | | IQTEMAATMETSIFTTEYQ | | | |
| 24 | h IL-2Ralpha | ELCDDDPPEIPHATFKAMAYKEGTMLNCECKRGFRRIKELVYMLCTGNSSHS | human | blocker | Wild-type |
| | (1-219) | SWDNQCQCTSSATRNTTKQVTPQPEEQKERKTTEMQSPMQPVDQASLPGHCR | | | residues 39- |
| | SGSL₃₉₋₄₂ | EPPPWENEATERIYHFVVGQMVYYQCVQGYRALHRGPAESVCKMTHGKTRWT | | | 42 replaced |
| | ELV | QPQLICTGEMETSQFPGEEKPQASPEGRPESETSCLVTTTDFQ | | | with ELV |
| | | IQTEMAATMETSIFTTEYQ | | | |
| 25 | h IL-2Ralpha | ELCDDDPPEIPHATFKAMAYKEGTMLNCECKRGFRRIKSGSLYMLCTGNSSH | human | blocker | Wild-type |
| | (1-192) | SSWDNQCQCTSSATRNTTKQVTPQPEEQKERKTTEMQSPMQPVDQASLPGHC | | | amino acids |
| | | REPPPWENEATERIYHFVVGQMVYYQCVQGYRALHRGPAESVCKMTHGKTRW | | | 1-192 |
| | | TQPQLICTGEMETSQFPGEEKPQASPEGRPESETSC | | | |
| 26 | h IL-2Ralpha | ELCDDDPPEIPHATFKAMAYKEGTILNCECKRGFRRIKSGSLYMLCTGNSSH | human | blocker | M25 to I |
| | (1-192)M25I | SSWDNQCQCTSSATRNTTKQVTPQPEEQKERKTTEMQSPMQPVDQASLPGHC | | | mutation |
| | | REPPPWENEATERIYHFVVGQMVYYQCVQGYRALHRGPAESVCKMTHGKTRW | | | |
| | | TQPQLICTGEMETSQFPGEEKPQASPEGRPESETSC | | | |
| 27 | h IL-2Ralpha | ELCDDDPPEIPHATFKAMAYKEGTMLNCECKRGFRRIKSGSVYMLCTGNSSH | human | blocker | L42 to V |
| | (1-192)L42V | SSWDNQCQCTSSATRNTTKQVTPQPEEQKERKTTEMQSPMQPVDQASLPGHC | | | mutation |
| | | REPPPWENEATERIYHFVVGQMVYYQCVQGYRALHRGPAESVCKMTHGKTRW | | | |
| | | TQPQLICTGEMETSQFPGEEKPQASPEGRPESETSC | | | |
| 28 | h IL-2Ralpha | ELCLYDPPEIPHATFKAMAYKEGTMLNCECKRGFRRIKSGSLYMLCTGNSSH | human | blocker | D4 to L |
| | (1- | SSWDNQCQCTSSATRNTTKQVTPQPEEQKERKTTEMQSPMQPVDQASLPGHC | | | mutation; |
| | 192)D4L/D5 | REPPPWENEATERIYHFVVGQMVYYQCVQGYRALHRGPAESVCKMTHGKTRW | | | D5 |
| | Y | TQPQLICTGEMETSQFPGEEKPQASPEGRPESETSC | | | to Y |
| | | | | | mutation |
| 29 | h IL-2Ralpha | ELCDDDPPEIPHATFKAMAYKEGTMLNCECKRGFRRIKELVYMLCTGNSSHS | human | blocker | Wild-type |
| | (1-192) | SWDNQCQCTSSATRNTTKQVTPQPEEQKERKTTEMQSPMQPVDQASLPGHCR | | | residues |
| | SGSL₃₉₋₄₂ | EPPPWENEATERIYHFVVGQMVYYQCVQGYRALHRGPAESVCKMTHGKTRWT | | | 39-42 |
| | ELV | QPQLICTGEMETSQFPGEEKPQASPEGRPESETSC | | | replaced |
| | | | | | with ELV |

IL-2 Blockers: anti-IL-2 sequences

| | | | | | |
|---|---|---|---|---|---|
| 30 | scFv2 | QSVLTQPPSVSGAPGQRVTISCTGTSSNIGAHYDVHWYQQFPGTAPKRLIYG | human | blocker | wild-type |
| | | NNNRPSGVPARFSGSKSGTSASLAITGLQAEDEADYYCQSYDRSLRGWVFGG | | | |
| | | GTKLTVLGEGKSSGSGSESKASEVQLVESGGGLVQPGRSLRLSCAASGFTFD | | | |
| | | DYAMHWVRQAPGKGLEWVSGISWNSGSIGYADSVKGRFTISRDNSKNTLYLQ | | | |
| | | MNSLRAEDTAVYYCAKDVNWNYGYYFDYWGQGTLVTVSS | | | |
| 31 | scFv2 (18mer | QSVLTQPPSVSGAPGQRVTISCTGTSSNIGAHYDVHWYQQFPGTAPKRLIYG | human | blocker | 18 mer |
| | linker) | NNNRPSGVPARFSGSKSGTSASLAITGLQAEDEADYYCQSYDRSLRGWVFGG | | | linker |
| | | GTKLTVLGGSTSGSGKPGSGEGSTKGEVQLVESGGGLVQPGRSLRLSCAASG | | | between VL |
| | | FTFDDYAMHWVRQAPGKGLEWVSGISWNSGSIGYADSVKGRFTISRDNSKNT | | | and VH |
| | | LYLQMNSLRAEDTAVYYCAKDVNWNYGYYFDYWGQGTLVTVSS | | | |
| 32 | VL region of | QSVLTQPPSVSGAPGQRVTISCTGTSSNIGAHYDVHWYQQFPGTAPKRLIYG | human | blocker | wild-type |
| | scFv2 | NNNRPSGVPARFSGSKSGTSASLAITGLQAEDEADYYCQSYDRSLRGWVFGG | | | |
| | | GTKLTVLG | | | |
| 33 | VH region of | EVQLVESGGGLVQPGRSLRLSCAASGFTFDDYAMHWVRQAPGKGLEWVSGIS | human | blocker | wild-type |
| | scFv2 | WNSGSIGYADSVKGRFTISRDNSKNTLYLQMNSLRAEDTAVYYCAKDVNWNY | | | |
| | | GYYFDYWGQGTLVTVSS | | | |
| 34 | scFv2 VL | TGTSSNIGAHYDVH | | | |
| | HVR1 | | | | |
| 35 | scFv2 VL | GNNNRPS | | | |
| | HVR2 | | | | |
| 36 | scFv2 VL | QSYDRSLRGWV | | | |
| | HVR3 | | | | |
| 37 | scFv2 VH | DDYAMH | | | |
| | HVR1 | | | | |
| 38 | scFv2 VH | GISWNSGSIGYADSVKG | | | |
| | HVR2 | | | | |
| 39 | scFv2 VH | KDVNWNYGYYFDY | | | |
| | HVR3 | | | | |
| 747 | scFv183 | DIVMTQSPDSLAVSLGERATINCKSSQSVLYSNNNKNYLAWYQQKPGQPPKL | human | blocker | linker |
| | | LIYGASTRESWVPDRFSGSGSGTDFTLTISSLQAEDVAVYYCQQWYYYPYTF | | | between |
| | | GQGTKVEIKGGGGSGGGGSGGGSGGGGSEVQLLESGGGLVQPGGSLRLSCAA | | | VL |
| | | SGFTFSSYYMSWVRQAPGKGLEWVSDISGRGGQTNYADSVKGRFTISRDNSK | | | and VH |
| | | NTLYLQMNSLRAEDTAVYYCARGGGSFANWGRGTLVTVSS | | | |
| 748 | VL region of | DIVMTQSPDSLAVSLGERATINCKSSQSVLYSNNNKNYLAWYQQKPGQPPKL | human | blocker | |
| | scFv183 | LIYGASTRESWVPDRFSGSGSGTDFTLTISSLQAEDVAVYYCQQWYYYPYTF | | | |
| | | GQGTKVEIK | | | |
| 749 | VH region of | EVQLLESGGGLVQPGGSLRLSCAASGFTFSSYYMSWVRQAPGKGLEWVSDIS | human | blocker | |
| | scFv183 | GRGGQTNYADSVKGRFTISRDNSKNTLYLQMNSLRAEDTAVYYCARGGGSFA | | | |
| | | NWGRGTLVTVSS | | | |
| 750 | scFv183 VL | KSSQSVLYSNNNKNYLA | | | |
| | HVR1 | | | | |
| 751 | scFv183 VL | GASTRES | | | |
| | HVR2 | | | | |
| 752 | scFv183 VL | QQWYYYPYT | | | |
| | HVR3 | | | | |
| 753 | scFv183 VH | SSYYMS | | | |
| | HVR1 | | | | |
| 754 | scFv183 VH | DISGRGGQTNYADSVKG | | | |
| | HVR2 | | | | |
| 755 | scFv183 VH | RGGGSFAN | | | |
| | HVR3 | | | | |
| 850 | 794B1P3B1 | EVQLVESGGGLVQAGGSLRLSCAASERTFNMNVMGWFRQAPGKEREFVAAIS | camelid | blocker | VHH |
| | | WSTGGTSYGNFVKGRFTISGDNAKNTVYLEMNSLKPEDTAEYYCAAARFFTS | | | |
| | | LGAGEYAYRGQGTQVTVSS | | | |
| 851 | 794B1P3A3 | QVQLVESGGGLVQAGDSLRLSCAPSGRTFGTYAPSRRTFGTYAMGWFRQAPG | camelid | blocker | VHH |
| | | KEREFVADITWSGDRTYYADSVKGRFTISRDNPKSTVYLQMSSLKPEDTAVY | | | |
| | | YCAADSFMSKVLAGSAEYWGQGTQVTVSS | | | |
| 852 | 794B1P3C3 | EVQLVESGGGLVQPGESLRLSCLASRTLSTFNVMAWYRQAPEKERELVAHVT | camelid | blocker | VHH |
| | | NGTTLVADSVKGRFTISRDYTKNTVDLQMSKLKPEDTAVYYCRFWRGRYEYW | | | |
| | | GQGTQVTVSS | | | |
| 853 | 794B1P3A4 | QVQLVESGGGLVQAGGSLRLSCAASVRTDSHNVVGWIRQAPGKEREFVAAIS | camelid | blocker | VHH |
| | | RSGYTSYTDSVKDRFTISRDNSRNTVYLQMNSLKPEDTALYYCAGRTFFSEF | | | |
| | | NVPPARNSGQGTQVTVSS | | | |
| 854 | 794B1P3B7 | QVQLVESGGGLVQPGGSLRLSCAASGRTFGTYAPSRRTFGTYAMGWFRQAPG | camelid | blocker | VHH |
| | | KEREFVADITWSGDRTYYADSVKGRFTISRDNPKSTVYLQMSSLKPEDTAVY | | | |
| | | YCAADSFMSKVLAGSAEYWGQGTQVTVSS | | | |
| 855 | 794B1P3C7 | EVQLVESGGGLVQAGGSLRLSCAASGRALYLMGWFRQVPGKEREFVAGILWS | camelid | blocker | VHH |
| | | SSRYADSVKGRFTISRDNAKNTVSLQMNSLKPEDTAVYYCAAAIRRGQDIAT | | | |
| | | ITSEYDYWGQGTQVTVSS | | | |
| 856 | 794B1P3G11 | QVQLVESGGGLVQAGGSPRLSCAASGRTLYFMGWFRQVPGKEREFVAGILWS | camelid | blocker | VHH |
| | | STTYADSVKGRFTISRDNAKNTASLQMNSLKPEDTAVYYCAAAIRRGQDIPT | | | |
| | | MTSEYAYWGQGTQVTVSS | | | |
| 863 | 794B1P3E1 | EVQLVESGGXLVQAGGSLRLSCAASERTFNMNVMGWFRQAPGKEREFVAAMS | camelid | block- | VHH |
| | | WSISGTSYGNSVKGRFTISGDNAKNTVYLEMNSLKPEDTAEYYCVAGRFFSS | | er | |
| | | LGAGDYAYRGQGTQVTVSS | | | |
| 864 | 794B1P3G7 | QVQLVESGGGLVQPGGSLRLSCAASGFTFADGVMAWVRQAPGKGHEWVSSIS | camelid | block- | VHH |
| | | ISVGSTSYADSVKGRFTISRDNAKNTLYLQMNSLKPEDTAVYYCAKARFFLQ | | er | |
| | | AGRLDFEYRGRGTQVTVSS | | | |
| 865 | 794B1P3G9 | EVQLVESGGGLVQAGDSLRLSCAPSGRTFGTYAPSRRTFGTCAMGWFRPATG | camelid | block- | VHH |
| | | REGDFVSYINWSGDRTYYAHSVKGRFTISRDNPKRTEYLQMNNRAPEDTAVY | | er | |
| | | YCAANTIMCKVVTGSAEYWEQGTQVTVSS | | | |
| 866 | 794B2P3G1 | QVQLVESGGGLVQAGGSLRLSCAASGRTFSNYFAGWFRQPPGEEREFVASIS | camelid | block- | VHH |
| | | WGGDRMFYTDAVKGRFTISRDNAKNTVDLQMNSLKPEDTAVYYCSADRFATA | | er | |
| | | LYNGNGNYWGQGTQVTVSS | | | |
| 867 | 794B2P3D2A | EVQLVESGGGLVQAGDSLRLSCAASGRTVSNYAMGWFRQAPGKGREWIVTSW | camelid | block- | VHH |
| | | TSGDARYEDSVKGRFTISRDHAKNTVYLQMNSLKPEDTGVYYCVADQFGSAI | | er | |
| | | LNGRAEYWGQGTQVTVSS | | | |
| 868 | 794B2P3D2B | EVQLVESGGGLVQAGGSLRLSCAASGRTFIGYTMGWFRQAPGKEREFVASII | camelid | block- | VHH |
| | | WRGDRTRYADSVKGRFTISGDNAKNTVYLRMNSMKPEDTAVYYCAARSGSHF | | er | |
| | | PSFDYWGQGTQVSVSS | | | |
| 869 | 794B2P3C10 | EVQLVESGGGLVQPGGSLRLSCEVSGRILYIMGWFRQVPGKDREFVAGILWS | camelid | block- | VHH |
| | | STKYGDFVNGRFTISRDNVKNTVSLQMNSLKPEDTAVYYCAAAIRRGQDIPT | | er | |
| | | ISSEYNYWGQGTQVTVSS | | | |
| 870 | 794B2P3F11 | EVQLVESGGGLVQPGGSLRLSCAASGSISSMNVMGWYRQAPGKQREFVAGMN | camelid | block- | VHH |
| | | SRSVTSYDDSVQGRFTVSRDHTKNMVYLQMNSLKPEDTAIYYCAYSTWWSTL | | er | |
| | | GNDVWGQGTQVTVSS | | | |

IL-10 Blockers: anti-IL-10 sequences

| | | | | | |
|---|---|---|---|---|---|
| 939 | scFv (VL- | DVVMTQSPLSLPVTLGQPASISCRSSQNIVHSNGNTYLEWYLQRPGQSPRLL | human | block- | anti- |
| | VH) | IYKVSNRFSGVPDRFSGSGSGTDFTLKISRVEAEDVGVYYCFQGSHVPWTFG | | er | IL10 |
| | | GQGTKVEIKGGGGSGGGSGGGGSEVQLVESGGGLVQPGGSLRLSCAASGFSF | | | |
| | | ATYGVHWVRQSPGKGLEWLGVIWRGGSTDYSAAFMSRLTISKDNSKNTVYLQ | | | |
| | | MNSLRAEDTAVYFCAKQAYGHYMDYWGQGTSVTVSS | | | |
| 940 | scFv (VH- | EVQLVESGGGLVQPGGSLRLSCAASGFSFATYGVHWVRQSPGKGLEWLGVIW | human | block- | anti- |
| | VL) | RGGSTDYSAAFMSRLTISKDNSKNTVYLQMNSLRAEDTAVYFCAKQAYGHYM | | er | IL10 |
| | | DYWGQGTSVTVSSGGGGSGGGGSGGGGSDVVMTQSPLSLPVTLGQPASISCR | | | |
| | | SSQNIVHSNGNTYLEWYLQRPGQSPRLLIYKVSNRFSGVPDRFSGSGSGTDF | | | |
| | | TLKISRVEAEDVGVYYCFQGSHVPWTFGQGTKVEIK | | | |
| 941 | scFv VL | DVVMTQSPLSLPVTLGQPASISCRSSQNIVHSNGNTYLEWYLQRPGQSPRLL | human | block- | anti- |
| | region | IYKVSNR | | er | IL10 VL |
| | | FSGVPDRFSGSGSGTDFTLKISRVEAEDVGVYYCFQGSHVPWTFGQGTKVEI | | | |
| | | K | | | |
| 942 | VL CDR1 | SSQNIVHSNGNTY | human | block- | anti- |
| | | | | er | IL10 VL |
| 943 | VL CDR2 | KVSNRFSGVPD | human | block- | anti- |
| | | | | er | IL10 VL |
| 944 | VL CDR3 | GSHVPW | human | block- | anti- |
| | | | | er | IL10 VL |
| 945 | scFv VH | EVQLVESGGGLVQPGGSLRLSCAASGFSFATYGVHWVRQSPGKGLEWLGVIW | human | block- | anti- |
| | region | RGGS | | er | IL10 VH |
| | | TDYSAAFMSRLTISKDNSKNTVYLQMNSLRAEDTAVYFCAKQAYGHYMDYWG | | | |
| | | QGTSVTVSS | | | |
| 946 | VH CDR1 | ASGFSFATYG | human | block- | anti- |
| | | | | er | IL10 VH |
| 947 | VH CDR2 | IWRGGSTDYSAAFMSR | human | block- | anti- |
| | | | | er | IL10 VH |
| 948 | VH CDR3 | QAYGHYMD | human | block- | anti- |
| | | | | er | IL10 VH |

IL-15 Blockers: anti-IL-15 sequences

| | | | | | |
|---|---|---|---|---|---|
| 949 | anti- | EVQLVQSGAEVKKPGESLKISCKVSGYFFTTYWIGWVRQMPGKGLEYMGIIY | human | block- | anti- |
| | IL15 | PGDSDTRYSPSFQGQVTISADKSISTAYLQWSSLKASDTAMYYCARGGNWNC | | er | IL15 |
| | heavy chain | FDYWGQGTLVTVSSASTKGPSVFPLAPSSKSTSGGTAALGCLVKDYFPEPVT | | | |
| | | VSWNSGALTSGVHTFPAVLQSSGLYSLSSVVTVPSSSLGTQTYICNVNHKPS | | | |
| | | NTKVDKRVEPKSCDKTHTCPPCPAPELLGGPSVFLFPPKPKDTLMISRTPEV | | | |
| | | TCVVVDVSHEDPEVKFNWYVDGVEVHNAKTKPREEQYNSTYRVVSVLTVLHQ | | | |
| | | DWLNGKEYKCKVSNKALPAPIEKTISKAKGQPREPQVYTLPPSRDELTKNQV | | | |
| | | SLTCLVKGFYPSDIAVEWESNGQPENNYKTTPPVLDSDGSFFLYSKLTVDKS | | | |
| | | RWQQGNVFSCSVMHEALHNHYTQKSLSLSPGK | | | |
| 950 | anti- | EVQLVQSGAEVKKPGESLKISCKVSGYFFTTYWIGWVRQMPGKGLEYMGIIY | human | block- | VH |
| | IL15 VH | PGDSDTRYSPSFQGQVTISADKSISTAYLQWSSLKASDTAMYYCARGGNWNC | | er | |
| | | FDYWGQGTLVTVSS | | | |
| 951 | anti- | EIVLTQSPGTLSLSPGERATLSCRASQSVSSSYLAWYQQKPGQAPRLLIYGA | human | block- | anti- |
| | IL15 | SRRATGIPDRFSGSGSGTDFTLTISRLEPEDFAVYYCQRYGSSHTFGQGTKL | | er | IL15 |
| | light chain | EISRTVAAPSVFIFPPSDEQLKSGTASVVCLLNNFYPREAKVQWKVDNALQS | | | |
| | | GNSQESVTEQDSKDSTYSLSSTLTLSKADYEKHKVYACEVTHQGLSSPVTKS | | | |
| | | FNRGEC | | | |
| 952 | anti- | EIVLTQSPGTLSLSPGERATLSCRASQSVSSSYLAWYQQKPGQAPRLLIYGA | human | block- | VL |
| | IL15 VL | SRRATGIPDRFSGSGSGTDFTLTISRLEPEDFAVYYCQRYGSSHTFGQGTKL | | er | |
| | | EISR | | | |
| 953 | ADL108-R3- | EIVLTQSPGTLSLSPGERATLSCRASQSVSSNALAWYQQKPGQAPRLLIYGA | human | block- | scFv |
| | 09 | SSRATGIPDRFSGSGSGTDFTLTISRLEPEDFAVYYCQQAGSYPITFGQGTK | | er | |
| | | VEIKGGGGSGGGGSGGGSGGGGSEVQLLESGGGLVQPGGSLRLSCAASGFTF | | | |
| | | TDYAMSWVRQAPGKGLEWVSGISGGGGSTRYADSVKGRFTISRDNSKNTLYL | | | |
| | | QMNSLRAEDTAVYYCARVVRGVISPYWYFDLWGRGTLVTVSS | | | |
| 954 | ADL108-R3- | EIVLTQSPGTLSLSPGERATLSCRASQSVSSNALAWYQQKPGQAPRLLIYGA | human | block- | scFv |
| | 09 VL | SSRATGIPDRFSGSGSGTDFTLTISRLEPEDFAVYYCQQAGSYPITFGQGTK | | er | |
| | | VEIK | | | |
| 955 | ADL108-R3- | EVQLLESGGGLVQPGGSLRLSCAASGFTFTDYAMSWVRQAPGKGLEWVSGIS | human | block- | scFv |
| | 09 VH | GGGGSTRYADSVKGRFTISRDNSKNTLYLQMNSLRAEDTAVYYCARVVRGVI | | er | |
| | | SPYWYFDLWGRGTLVTVSS | | | |
| 956 | ADL108-R3- | EIVLTQSPGTLSLSPGERATLSCRASQSVSSNYLAWYQQKPGQAPRLLIYGA | human | block- | scFv |
| | 61 | SSRATGIPDRFSGSGSGTDFTLTISRLEPEDFAVYYCQQYSSSPFTFGQGTK | | er | |
| | | VEIKGGGGSGGGGSGGGSGGGGSEVQLLESGGGLVQPGGSLRLSCAASGFTF | | | |
| | | TDYWMSWVRQAPGKGLEWVSGIDGYGGGTNYADSVKGRFTISRDNSKNTLYL | | | |
| | | QMNSLRAEDTAVYYCAKARDSYADYWGQGTLVTVSS | | | |
| 957 | ADL108-R3- | EVQLLESGGGLVQPGGSLRLSCAASGFTFTDYWMSWVRQAPGKGLEWVSGID | human | block- | scFv |
| | 61 VH | GYGGGTNYADSVKGRFTISRDNSKNTLYLQMNSLRAEDTAVYYCAKARDSYA | | er | |
| | | DYWGQGTLVTVSS | | | |
| 958 | ADL108-R3- | EIVLTQSPGTLSLSPGERATLSCRASQSVSSNYLAWYQQKPGQAPRLLIYGA | human | block- | scFv |
| | 61 VL | SSRATGIPDRFSGSGSGTDFTLTISRLEPEDFAVYYCQQYSSSPFTFGQGTK | | er | |
| | | VEIK | | | |
| 959 | ADL108-R3- | DIVMTQSPDSLAVSLGERATINCKSSQSVLYSSNNENDLAWYQQKPGQPPKL | human | block- | scFv |
| | 79 | LIYDASTRESGVPDRFSGSGSGTDFTLTISSLQAEDVAVYYCQQYSYRPLTF | | er | |
| | | GQGTKVEIKGGGGSGGGGSGGGSGGGGSEVQLLESGGGLVQPGGSLRLSCAA | | | |
| | | SGFTFSDTAMSWVRQAPGKGLEWVSAISGSGGSTYYADSVKGRFTISRDNSK | | | |
| | | NTLYLQMNSLRAEDTAVYYCANEWIPYGDYAFWGQGSLVTVSS | | | |
| 960 | ADL108-R3- | EVQLLESGGGLVQPGGSLRLSCAASGFTFSDTAMSWVRQAPGKGLEWVSAIS | human | block- | scFv |
| | 79 VH | GSGGSTYYADSVKGRFTISRDNSKNTLYLQMNSLRAEDTAVYYCANEWIPYG | | er | |
| | | DYAFWGQGSLVTVSS | | | |
| 961 | ADL108-R3- | DIVMTQSPDSLAVSLGERATINCKSSQSVLYSSNNENDLAWYQQKPGQPPKL | human | block- | scFv |
| | 79 VL | LIYDASTRESGVPDRFSGSGSGTDFTLTISSLQAEDVAVYYCQQYSYRPLTF | | er | |
| | | GQGTKVEIK | | | |
| 962 | ADL108-R3- | DIVMTQSPDSLAVSLGERATINCKSSQSVLYSSNNKNYLAWYQQKPGQPPKL | human | block- | scFv |
| | 07 | LIYGASTRESGVPDRFSGSGSGTDFTLTISSLQAEDVAVYYCQQGYSAPFTF | | er | |
| | | GQGTKVEIKGGGGSGGGGSGGGSGGGGSEVQLLESGGGLVQPGGSLRLSCAA | | | |
| | | SGFTFTDTYMSWVRQAPGKGLEWVSAISGYGDTTKYADSVKGRFTISRDNSK | | | |
| | | NTLYLQMNSLRAEDTAVYYCARDRTASRFGYWGQGTLVTVSS | | | |
| 963 | ADL108-R3- | EVQLLESGGGLVQPGGSLRLSCAASGFTFTDTYMSWVRQAPGKGLEWVSAIS | human | block- | scFv |
| | 07 VH | GYGDTTKYADSVKGRFTISRDNSKNTLYLQMNSLRAEDTAVYYCARDRTASR | | er | |
| | | FGYWGQGTLVTVSS | | | |
| 964 | ADL108-R3- | DIVMTQSPDSLAVSLGERATINCKSSQSVLYSSNNKNYLAWYQQKPGQPPKL | human | block- | scFv |
| | 07 VL | LIYGASTRESGVPDRFSGSGSGTDFTLTISSLQAEDVAVYYCQQGYSAPFTF | | er | |
| | | GQGTKVEIK | | | |
| 965 | ADL108-R3- | DIVMTQSPDSLAVSLGERATINCKSSQSVLYSGNNENYLAWYQQKPGQPPKL | human | block- | scFv |
| | 10 | LIYAASTRESGVPDRFSGSGSGTDFTLTISSLQAEDVAVYYCQQYQENPITF | | er | |
| | | GQGTKVEIKGGGGSGGGGSGGGSGGGGSEVQLLESGGGLVQPGGSLRLSCAA | | | |
| | | SGFTFSNYAMSWVRQAPGKGLEWVSGISGGGGSTDYADSVKGRFTISRDNSK | | | |
| | | NTLYLQMNSLRAEDTAVYYCARWPYGHWGQGTLVTVSS | | | |
| 966 | ADL108-R3- | EVQLLESGGGLVQPGGSLRLSCAASGFTFSNYAMSWVRQAPGKGLEWVSGIS | human | block- | scFv |
| | 10 VH | GGGGSTDYADSVKGRFTISRDNSKNTLYLQMNSLRAEDTAVYYCARWPYGHW | | er | |
| | | GQGTLVTVSS | | | |
| 967 | ADL108-R3- | DIVMTQSPDSLAVSLGERATINCKSSQSVLYSGNNENYLAWYQQKPGQPPKL | human | block- | scFv |
| | 10 VL | LIYAASTRESGVPDRFSGSGSGTDFTLTISSLQAEDVAVYYCQQYQENPITF | | er | |
| | | GQGTKVEIK | | | |
| 968 | ADL108-R3- | DIVMTQSPDSLAVSLGERATINCKSSQSVLDSYNNKNDLAWYQQKPGQPPKL | human | block- | scFv |
| | 30 | LIYAASTRESGVPDRFSGSGSGTDFTLTISSLQAEDVAVYYCQQYYEAPYTF | | er | |
| | | GQGTKVEIKGGGGSGGGGSGGGSGGGGSEVQLLESGGGLVQPGGSLRLSCAA | | | |
| | | SGFTFSSYYMSWVRQAPGKGLEWVSEISGSGDSTYYADSVKGRFTISRDNSK | | | |
| | | NTLYLQMNSLRAEDTAVYYCASYYYYGSGFDYWGQGTLVTVSS | | | |
| 969 | ADL108-R3- | DIVMTQSPDSLAVSLGERATINCKSSQSVLDSYNNKNDLAWYQQKPGQPPKL | human | block- | scFv |
| | 30 VH | LIYAASTRESGVPDRFSGSGSGTDFTLTISSLQAEDVAVYYCQQYYEAPYTF | | er | |
| | | GQGTKVEIK | | | |
| 970 | ADL108-R3- | EVQLLESGGGLVQPGGSLRLSCAASGFTFSSYYMSWVRQAPGKGLEWVSEIS | human | block- | scFv |
| | 30 VL | GSGDSTYYADSVKGRFTISRDNSKNTLYLQMNSLRAEDTAVYYCASYYYYGS | | er | |
| | | GFDYWGQGTLVTVSS | | | |
| 971 | ADL108-R3- | DIVMTQSPDSLAVSLGERATINCKSSQSVLHSSNNENDLAWYQQKPGQPPKL | human | block- | scFv |
| | 38 | LIYAASTRESGVPDRFSGSGSGTDFTLTISSLQAEDVAVYYCQQWYSEPYTF | | er | |
| | | GQGTKVEIKGGGGSGGGGSGGGSGGGGSEVQLLESGGGLVQPGGSLRLSCAA | | | |
| | | SGFTFSSTYMSWVRQAPGKGLEWVSGIYGGGTSYADSVKGRFTISRDNSKNT | | | |
| | | LYLQMNSLRAEDTAVYYCARENYYDILTGYYTQTETWGQGTLVTVSS | | | |
| 972 | ADL108-R3- | DIVMTQSPDSLAVSLGERATINCKSSQSVLHSSNNENDLAWYQQKPGQPPKL | human | block- | scFv |
| | 38 VH | LIYAASTRESGVPDRFSGSGSGTDFTLTISSLQAEDVAVYYCQQWYSEPYTF | | er | |
| | | GQGTKVEIK | | | |
| 973 | ADL108-R3- | EVQLLESGGGLVQPGGSLRLSCAASGFTFSSTYMSWVRQAPGKGLEWVSGIY | human | block- | scFv |
| | 38 VL | GGGTSYADSVKGRFTISRDNSKNTLYLQMNSLRAEDTAVYYCARENYYDILT | | er | |
| | | GYYTQTETWGQGTLVTVSS | | | |
| 974 | ADL108-R3- | EIVLTQSPGTLSLSPGERATLSCRASQSVSSNALAWYQQKPGQAPRLLIYGA | human | block- | scFv |
| | 43 | SSRATGIPDRFSGSGSGTDFTLTISRLEPEDFAVYYCQQYSEAPITFGQGTK | | er | |
| | | VEIKGGGGSGGGGSGGGSGGGGSEVQLLESGGGLVQPGGSLRLSCAASGFTF | | | |
| | | TSYAMSWVRQAPGKGLEXVSGIDGYGGSTYYADSVKGRFTISRDNSKNTLYL | | | |
| | | QMNSLRAEDTAVYYCARAGIHLYDYWGQGTLVTVSS | | | |
| 975 | ADL108-R3- | EIVLTQSPGTLSLSPGERATLSCRASQSVSSNALAWYQQKPGQAPRLLIYGA | human | block- | scFv |
| | 43 VH | SSRATGIPDRFSGSGSGTDFTLTISRLEPEDFAVYYCQQYSEAPITFGQGTK | | er | |
| | | VEIK | | | |
| 976 | ADL108-R3- | EVQLLESGGGLVQPGGSLRLSCAASGFTFTSYAMSWVRQAPGKGLEXVSGID | human | block- | scFv |
| | 43 VL | GYGGSTYYADSVKGRFTISRDNSKNTLYLQMNSLRAEDTAVYYCARAGIHLY | | er | |
| | | DYWGQGTLVTVSS | | | |
| 977 | ADL108-R3- | EIVLTQSPGTLSLSPGERATLSCRASQSVSSSSLAWYQQKPGQAPRLLIYAA | human | block- | scFv |
| | 53 | SSRATGIPDRFSGSGSGTDFTLTISRLEPEDFAVYYCQQDSSSPFTFGQGTK | | er | |
| | | VEIKGGGGSGGGGSGGGSGGGGSEVQLLESGGGLVQPGGSLRLSCAASGFTF | | | |
| | | SSYAMSWVRQAPGKGLEWVSAISGRGDYTKYADSVKGRFTISRDNSKNTLYL | | | |
| | | QMNSLRAEDTAVYYCARGTTIFGVTAFVYWGQGTLVTVSS | | | |
| 978 | ADL108-R3- | EVQLLESGGGLVQPGGSLRLSCAASGFTFSSYAMSWVRQAPGKGLEWVSAIS | human | block- | scFv |
| | 53 VH | GRGDYTKYADSVKGRFTISRDNSKNTLYLQMNSLRAEDTAVYYCARGTTIFG | | er | |
| | | VTAFVYWGQGTLVTVSS | | | |
| 979 | ADL108-R3- | EIVLTQSPGTLSLSPGERATLSCRASQSVSSSSLAWYQQKPGQAPRLLIYAA | human | block- | scFv |
| | 53 VL | SSRATGIPDRFSGSGSGTDFTLTISRLEPEDFAVYYCQQDSSSPFTFGQGTK | | er | |
| | | VEIK | | | |
| 980 | ADL108-R3- | EIVLTQSPGTLSLSPGERATLSCRASQSVQSSALAWYQQKPGQAPRLLIYGA | human | block- | scFv |
| | 60 | SSRATGIPDRFSGSGSGTDFTLTISRLEPEDFAVYYCQQDGSWPLTFGQGTK | | er | |
| | | VEIKGGGGSGGGGSGGGSGGGGSEVQLLESGGGLVQPGGSLRLSCAASGFTF | | | |
| | | SDYAMSWVRQAPGKGLEWVSRIDGGGGYTDYADSVKGRFTISRDNSKNTLYL | | | |
| | | QMNSLRAEDTAVYYCARHGSATIFGVVIHGYWYFDLWGRGTLVTVSS | | | |
| 981 | ADL108-R3- | EVQLLESGGGLVQPGGSLRLSCAASGFTFSDYAMSWVRQAPGKGLEWVSRID | human | block- | scFv |
| | 60 VH | GGGGYTDYADSVKGRFTISRDNSKNTLYLQMNSLRAEDTAVYYCARHGSATI | | er | |
| | | FGVVIHGYWYFDLWGRGTLVTVSS | | | |
| 982 | ADL108-R3- | EIVLTQSPGTLSLSPGERATLSCRASQSVQSSALAWYQQKPGQAPRLLIYGA | human | block- | scFv |
| | 60 VL | SSRATGIPDRFSGSGSGTDFTLTISRLEPEDFAVYYCQQDGSWPLTFGQGTK | | er | |
| | | VEIK | | | |
| 983 | ADL108-R3- | DIVMTQSPDSLAVSLGERATINCKSSQSVLRSSNNKNNLAWYQQKPGQPPKL | human | block- | scFv |
| | 87 | LIYWASTRESGVPDRFSGSGSGTDFTLTISSLQAEDVAVYYCQQSYYEPITF | | er | |
| | | GQGTKVEIKGGGGSGGGGSGGGSGGGGSEVQLLESGGGLVQPGGSLRLSCAA | | | |
| | | SGFTFSDTAMSWVRQAPGKGLEWVSGISGGGGYTNYADSVKGRFTISRDNSK | | | |
| | | NTLYLQMNSLRAEDTAVYYCAKSPDYDRRNYYDHWGQGTLVTVSS | | | |
| 984 | ADL108-R3- | DIVMTQSPDSLAVSLGERATINCKSSQSVLRSSNNKNNLAWYQQKPGQPPKL | human | block- | scFv |
| | 87 VL | LIYWASTRESGVPDRFSGSGSGTDFTLTISSLQAEDVAVYYCQQSYYEPITF | | er | |
| | | GQGTKVEIK | | | |
| 985 | ADL108-R3- | EVQLLESGGGLVQPGGSLRLSCAASGFTFSDTAMSWVRQAPGKGLEWVSGIS | human | block- | scFv |
| | 87 VH | GGGGYTNYADSVKGRFTISRDNSKNTLYLQMNSLRAEDTAVYYCAKSPDYDR | | er | |
| | | RNYYDHWGQGTLVTVSS | | | |
| 986 | ADL108-R3- | DIVMTQSPDSLAVSLGERATINCKSSQSVLYSGNNENYLAWYQQKPGQPPKL | human | block- | scFv |
| | 90 | LIYDASTRESGVPDRFSGSGSGTDFTLTISSLQAEDVAVYYCQQWSNYPYTF | | er | |
| | | GQGTKVEIKGGGGSGGGGSGGGSGGGGSEVQLLESGGGLVQPGGSLRLSCAA | | | |
| | | SGFTFTDTYMSWVRQAPGKGLEWVSRIDGRGGGTYYADSVKGRFTISRDNSK | | | |
| | | NTLYLQMNSLRAEDTAVYYC | | | |
| 987 | ADL108-R3- | DIVMTQSPDSLAVSLGERATINCKSSQSVLYSGNNENYLAWYQQKPGQPPKL | human | block- | scFv |
| | 90 VL | LIYDASTRESGVPDRFSGSGSGTDFTLTISSLQAEDVAVYYCQQWSNYPYTF | | er | |
| | | GQGTKVEIK | | | |
| 988 | ADL108-R3- | EVQLLESGGGLVQPGGSLRLSCAASGFTFTDTYMSWVRQAPGKGLEWVSRID | human | block- | scFv |
| | 90 VH | GRGGGTYYADSVKGRFTISRDNSKNTLYLQMNSLRAEDTAVYYCAKGGSYGY | | er | |
| | | WGQGTLVTVSS | | | |

CXCL10 Blockers: anti-CXCL10 sequences

| | | | | | |
|---|---|---|---|---|---|
| 989 | anti-CXCL10 | GIQCEVKLVESGGGLVQPGGSLRLSCATSGFTFTDYYMSWVRQPPGKALEWL | human | block- | anti- |
| | scFv VL-VH | GFIRNKANGYTTEYSASVKGRFTISRDNQSILYLQMNTLRAEDSATYYCARD | | er | CXCL10 |
| | | PTIGTVLCYGLLGSRNLSGGGGSGGGGSGGGGSEVQLQQSGPELEKPGASVK | | | scFv |
| | | ISCKASGYSFTGYNMNWVKQSNGKSLEWIGNIDPYYGGTSYNQKFKGKATLT | | | |
| | | VDKSSSTAYMQLKSLTSEDSAVYYCARSGTAWFAYWGQGTLV | | | |
| 990 | anti-CXCL10 | EVQLQQSGPELEKPGASVKISCKASGYSFTGYNMNWVKQSNGKSLEWIGNID | human | block- | anti- |
| | scFv VH-VL | PYYGGTSYNQKFKGKATLTVDKSSSTAYMQLKSLTSEDSAVYYCARSGTAWF | | er | CXCL10 |
| | | AYWGQGTLVGGGGSGGGGSGGGGSGIQCEVKLVESGGGLVQPGGSLRLSCAT | | | scFv |
| | | SGFTFTDYYMSWVRQPPGKALEWLGFIRNKANGYTTEYSASVKGRFTISRDN | | | |
| | | QSILYLQMNTLRAEDSATYYCARDPTIGTVLCYGLLGSRNLS | | | |
| 991 | anti-CXCL10 | EVQLQQSGPELEKPGASVKISCKASGYSFTGYNMNWVKQSNGKSLEWIGNID | human | block- | anti- |
| | scFv VH | PYYGGTSYNQKFKGKATLTVDKSSSTAYMQLKSLTSEDSAVYYCARSGTAWF | | er | CXCL10 |
| | | AYWGQGTLV | | | |
| 992 | anti-CXCL10 | GIQCEVKLVESGGGLVQPGGSLRLSCATSGFTFTDYYMSWVRQPPGKALEWL | human | block- | anti- |
| | scFv VL | GFIRNKANGYTTEYSASVKGRFTISRDNQSILYLQMNTLRAEDSATYYCARD | | er | CXCL10 |
| | | PTIGTVLCYGLLGSRNLS | | | |
| 993 | VH CDR1 | GYNMN | human | block- | anti- |
| | | | | er | CXCL10 |
| 994 | VH CDR2 | NIDPYYGGTSYNQKFK | human | block- | anti- |
| | | | | er | CXCL10 |
| 995 | VH CDR3 | SGTAWFAYW | human | block- | anti- |
| | | | | er | CXCL10 |
| 996 | VL CDR1 | ATSGFTFTDYYMS | human | block- | anti- |
| | | | | er | CXCL10 |
| 997 | VL CDR2 | IRNKANGYTTEYSA | | | anti- |
| | | | | | CXCL10 |
| 998 | VL CDR3 | ARDPTIGTV | human | block- | anti- |
| | | | | er | CXCL10 |

TGF-β Blockers: anti-TGF-β sequences

| | | | | | |
|---|---|---|---|---|---|
| 999 | VH region | QLQVQESGPGLVKPSETLSLTCTVSGGSISNSYFSWGWIRQPPGKGLEWIGS | human | TGFb | antibody |
| | TGF1 | FYYGEKTYYNPSLKSRATISIDTSKSQFSLKLSSVTAADTAVYYCPRGPTMI | | trap | fragment |
| | | RGVIDSWGQGTLVTVSS | | | to |
| | | | | | TGFbeta |
| | | | | | Receptor |
| | | | | | II |
| 1000 | VL region | EIVLTQSPATLSLSPGERATLSCRASQSVRSYLAWYQQKPGQAPRLLIYDAS | human | TGFb | antibody |
| | TGF1 | NRATGIPARFSGSGSGTDFTLTISSLEPEDFAVYYCQQRSNWPPTFGQGTKV | | trap | fragment |
| | | EIK | | | to |
| | | | | | TGFbeta |
| | | | | | Receptor |
| | | | | | II |
| 1001 | scFv TGF1 | QLQVQESGPGLVKPSETLSLTCTVSGGSISNSYFSWGWIRQPPGKGLEWIGS | human | TGFb | antibody |
| | (VH-VL) | FYYGEKTYYNPSLKSRATISIDTSKSQFSLKLSSVTAADTAVYYCPRGPTMI | | trap | fragment |
| | | RGVIDSWGQGTLVTVSSGGGGSGGGGSGGGGSEIVLTQSPATLSLSPGERAT | | | to |
| | | LSCRASQSVRSYLAWYQQKPGQAPRLLIYDASNRATGIPARFSGSGSGTDFT | | | TGFbeta |
| | | LTISSLEPEDFAVYYCQQRSNWPPTFGQGTKVEIK | | | Receptor |
| | | | | | II |
| 1002 | scFv TGF1 | EIVLTQSPATLSLSPGERATLSCRASQSVRSYLAWYQQKPGQAPRLLIYDAS | human | TGFb | antibody |
| | (VL-VH) | NRATGIPARFSGSGSGTDFTLTISSLEPEDFAVYYCQQRSNWPPTFGQGTKV | | trap | fragment |
| | | EIKGGGGSGGGGSGGGGSQLQVQESGPGLVKPSETLSLTCTVSGGSISNSYF | | | to |
| | | SWGWIRQPPGKGLEWIGSFYYGEKTYYNPSLKSRATISIDTSKSQFSLKLSS | | | TGFbeta |
| | | VTAADTAVYYCPRGPTMIRGVIDSWGQGTLVTVSS | | | Receptor |
| | | | | | II |
| 1003 | VH region | QLQLQESGPGLVKPSETLSLTCTVSGGSISSSSYSWGWIRQPPGKGLEWIGS | human | TGFb | antibody |
| | TGF3 | FYYSGITYYSPSLKSRIIISEDTSKNQFSLKLSSVTAADTAVYYCASGFTMI | | trap | fragment |
| | | RGALDYWGQGTLVTVSS | | | to |
| | | | | | TGFbeta |
| | | | | | Receptor |
| | | | | | II |
| 1004 | VL region | EIVLTQSPATLSLSPGERATLSCRASQSVRSFLAWYQQKPGQAPRLLIYDAS | human | TGFb | antibody |
| | TGF3 | NRATGIPARFSGSGSGTDFTLTISSLEPEDFAVYYCQQRSNWPPTFGQGTKV | | trap | fragment |
| | | EIK | | | to |
| | | | | | TGFbeta |
| | | | | | Receptor |
| | | | | | II |
| 1005 | scFv TGF3 | QLQLQESGPGLVKPSETLSLTCTVSGGSISSSSYSWGWIRQPPGKGLEWIGS | human | TGFb | antibody |
| | (VH-VL) | FYYSGITYYSPSLKSRIIISEDTSKNQFSLKLSSVTAADTAVYYCASGFTMI | | trap | fragment |
| | | RGALDYWGQGTLVTVSSGGGGSGGGGSGGGGSEIVLTQSPATLSLSPGERAT | | | to |
| | | LSCRASQSVRSFLAWYQQKPGQAPRLLIYDASNRATGIPARFSGSGSGTDFT | | | TGFbeta |
| | | LTISSLEPEDFAVYYCQQRSNWPPTFGQGTKVEIK | | | Receptor |
| | | | | | II |
| 1006 | scFv TGF3 | EIVLTQSPATLSLSPGERATLSCRASQSVRSFLAWYQQKPGQAPRLLIYDAS | human | TGFb | antibody |
| | (VL-VH) | NRATGIPARFSGSGSGTDFTLTISSLEPEDFAVYYCQQRSNWPPTFGQGTKV | | trap | fragment |
| | | EIKGGGGSGGGGSGGGGSQLQLQESGPGLVKPSETLSLTCTVSGGSISSSSY | | | to |
| | | SWGWIRQPPGKGLEWIGSFYYSGITYYSPSLKSRIIISEDTSKNQFSLKLSS | | | TGFbeta |
| | | VTAADTAVYYCASGFTMIRGALDYWGQGTLVTVSS | | | Receptor |
| | | | | | II |
| 1007 | Fresolimumab | QVQLVQSGAEVKKPGSSVKVSCKASGYTFSSNVISWVRQAPGQGLEWMGGVI | human | TGFb | antibody |
| | heavy chain | PIVDIANYAQRFKGRVTITADESTSTTYMELSSLRSEDTAVYYCASTLGLVL | | trap | targeting |
| | | DAMDYWGQGTLVTVSSASTKGPSVFPLAPCSRSTSESTAALGCLVKDYFPEP | | | TGFb1, 2, 3 |
| | | VTVSWNSGALTSGVHTFPAVLQSSGLYSLSSVVTVPSSSLGTKTYTCNVDHK | | | |
| | | PSNTKVDKRVESKYGPPCPSCPAPEFLGGPSVFLFPPKPKDTLMISRTPEVT | | | |
| | | CVVVDVSQEDPEVQFNWYVDGVEVHNAKTKPREEQFNSTYRVVSVLTVLHQD | | | |
| | | WLNGKEYKCKVSNKGLPSSIEKTISKAKGQPREPQVYTLPPSQEEMTKNQVS | | | |
| | | LTCLVKGFYPSDIAVEWESNGQPENNYKTTPPVLDSDGSFFLYSRLTVDKSR | | | |
| | | WQEGNVFSCSVMHEALHNHYTQKSLSLSLGK | | | |
| 1008 | Fresolimumab | QVQLVQSGAEVKKPGSSVKVSCKASGYTFSSNVISWVRQAPGQGLEWMGGVI | human | TGFb | antibody |
| | VH | PIVDIANYAQRFKGRVTITADESTSTTYMELSSLRSEDTAVYYCASTLGLVL | | trap | targeting |
| | | DAMDYWGQGTLVTVSS | | | TGFb1, 2, 3 |
| 1009 | Fresolimumab | ETVLTQSPGTLSLSPGERATLSCRASQSLGSSYLAWYQQKPGQAPRLLIYGA | human | TGFb | antibody |
| | light chain | SSRAPGIPDRFSGSGSGTDFTLTISRLEPEDFAVYYCQQYADSPITFGQGTR | | trap | targeting |
| | | LEIKRTVAAPSVFIFPPSDEQLKSGTASVVCLLNNFYPREAKVQWKVDNALQ | | | TGFb1, 2, 3 |
| | | SGNSQESVTEQDSKDSTYSLSSTLTLSKADYEKHKVYACEVTHQGLSSPVTK | | | |
| | | SFNRGEC | | | |
| 1010 | Fresolimumab | ETVLTQSPGTLSLSPGERATLSCRASQSLGSSYLAWYQQKPGQAPRLLIYGA | human | TGFb | antibody |
| | VL | SSRAPGIPDRFSGSGSGTDFTLTISRLEPEDFAVYYCQQYADSPITFGQGTR | | trap | targeting |
| | | LEIK | | | TGFb1, 2, 3 |

Blockers: IL-2R sequences

| | | | | | |
|---|---|---|---|---|---|
| 40 | h IL-2Ralpha | ELCLYDPPEIPHATFKAMAYKEGTILNCECKRGFRRIKSGSLYMLCTGNSSH | human | block- | D4 to L |
| | (1- | SSWDNQCQCTSSATRNTTKQVTPQPEEQKERKTTEMQSPMQPVDQASLPGHC | | er | mutation; D5 |
| | 192)M25I/D4 | REPPPWENEATERIYHFVVGQMVYYQCVQGYRALHRGPAESVCKMTHGKTRW | | | to Y |
| | L/D5Y | TQPQLICTGEMETSQFPGEEKPQASPEGRPESETSC | | | mutation; |
| | | | | | M25 to I |
| | | | | | mutation |
| 41 | h IL-2Ralpha | ELCDDDPPEIPHATFKAMAYKEGTILNCECKRGFRRIKSGSVYMLCTGNSSH | human | block- | M25 to I |
| | (1- | SSWDNQCQCTSSATRNTTKQVTPQPEEQKERKTTEMQSPMQPVDQASLPGHC | | er | mutation; |
| | 192)M25I/L4 | REPPPWENEATERIYHFVVGQMVYYQCVQGYRALHRGPAESVCKMTHGKTRW | | | L42 to V |
| | 2V | TQPQLICTGEMETSQFPGEEKPQASPEGRPESETSC | | | mutation |
| 42 | h IL-2Ralpha | ELCLYDPPEIPHATFKAMAYKEGTMLNCECKRGFRRIKSGSVYMLCTGNSSH | human | block- | D4 to L |
| | (1-192) | SSWDNQCQCTSSATRNTTKQVTPQPEEQKERKTTEMQSPMQPVDQASLPGHC | | er | mutation; D5 |
| | D4L/D5Y/L4 | REPPPWENEATERIYHFVVGQMVYYQCVQGYRALHRGPAESVCKMTHGKTRW | | | to Y |
| | 2V | TQPQLICTGEMETSQFPGEEKPQASPEGRPESETSC | | | mutation; |
| | | | | | L42 to V |
| | | | | | mutation |
| 43 | h IL-2Ralpha | ELCLYDPPEIPHATFKAMAYKEGTILNCECKRGFRRIKSGSVYMLCTGNSSH | human | block- | D4 to L |
| | (1-192) | SSWDNQCQCTSSATRNTTKQVTPQPEEQKERKTTEMQSPMQPVDQASLPGHC | | er | mutation; D5 |
| | M25I/D4L/D5 | REPPPWENEATERIYHFVVGQMVYYQCVQGYRALHRGPAESVCKMTHGKTRW | | | to Y |
| | Y/L42V | TQPQLICTGEMETSQFPGEEKPQASPEGRPESETSC | | | mutation; |
| | | | | | M25 to I |
| | | | | | mutation; |
| | | | | | L42 to V |
| | | | | | mutation |
| 44 | h IL-2Ralpha | ELCDDDPPEIPHATFKAMAYKEGTMLNCECKRGFRRIKSGSLYMLCTGNSSH | human | block- | Wild-type |
| | (1-178) | SSWDNQCQCTSSATRNTTKQVTPQPEEQKERKTTEMQSPMQPVDQASLPGHC | | er | amino acids |
| | | REPPPWENEATERIYHFVVGQMVYYQCVQGYRALHRGPAESVCKMTHGKTRW | | | 1-178 |
| | | TQPQLICTGEMETSQFPGEEKP | | | |
| 45 | h IL-2Ralpha | ELCDDDPPEIPHATFKAMAYKEGTILNCECKRGFRRIKSGSLYMLCTGNSSH | human | block- | M25 to I |
| | (1-178) M25I | SSWDNQCQCTSSATRNTTKQVTPQPEEQKERKTTEMQSPMQPVDQASLPGHC | | er | mutation |
| | | REPPPWENEATERIYHFVVGQMVYYQCVQGYRALHRGPAESVCKMTHGKTRW | | | |
| | | TQPQLICTGEMETSQFPGEEKP | | | |
| 46 | h IL-2Ralpha | ELCDDDPPEIPHATFKAMAYKEGTMLNCECKRGFRRIKSGSVYMLCTGNSSH | human | block- | L42 to V |
| | (1-178) L42V | SSWDNQCQCTSSATRNTTKQVTPQPEEQKERKTTEMQSPMQPVDQASLPGHC | | er | mutation |
| | | REPPPWENEATERIYHFVVGQMVYYQCVQGYRALHRGPAESVCKMTHGKTRW | | | |
| | | TQPQLICTGEMETSQFPGEEKP | | | |
| 47 | h IL-2Ralpha | ELCLYDPPEIPHATFKAMAYKEGTMLNCECKRGFRRIKSGSLYMLCTGNSSH | human | block- | D4 to L |
| | (1-178) | SSWDNQCQCTSSATRNTTKQVTPQPEEQKERKTTEMQSPMQPVDQASLPGHC | | er | mutation; D5 |
| | D4L/D5Y | REPPPWENEATERIYHFVVGQMVYYQCVQGYRALHRGPAESVCKMTHGKTRW | | | to Y |
| | | TQPQLICTGEMETSQFPGEEKP | | | mutation |
| 48 | h IL-2Ralpha | ELCDDDPPEIPHATFKAMAYKEGTMLNCECKRGFRRIKELVYMLCTGNSSHS | human | block- | Wild-type |
| | (1-178) | SWDNQCQCTSSATRNTTKQVTPQPEEQKERKTTEMQSPMQPVDQASLPGHCR | | er | residues 39- |
| | SGSL₃₉₋₄₂ | EPPPWENEATERIYHFVVGQMVYYQCVQGYRALHRGPAESVCKMTHGKTRWT | | | 42 replaced |
| | ELV | QPQLICTGEMETSQFPGEEKP | | | with ELV |
| 49 | h IL-2Ralpha | ELCDDDPPEIPHATFKAMAYKEGTILNCECKRGFRRIKSGSVYMLCTGNSSH | human | block- | M25 to I |
| | (1-178) | SSWDNQCQCTSSATRNTTKQVTPQPEEQKERKTTEMQSPMQPVDQASLPGHC | | er | mutation; |
| | M25I/L42V | REPPPWENEATERIYHFVVGQMVYYQCVQGYRALHRGPAESVCKMTHGKTRW | | | L42 to V |
| | | TQPQLICTGEMETSQFPGEEKP | | | mutation |
| 50 | h IL-2Ralpha | ELCLYDPPEIPHATFKAMAYKEGTMLNCECKRGFRRIKSGSVYMLCTGNSSH | human | block- | D4 to L |
| | (1-178) | SSWDNQCQCTSSATRNTTKQVTPQPEEQKERKTTEMQSPMQPVDQASLPGHC | | er | mutation; D5 |
| | D4L/D5Y/L4 | REPPPWENEATERIYHFVVGQMVYYQCVQGYRALHRGPAESVCKMTHGKTRW | | | to Y |
| | 2V | TQPQLICTGEMETSQFPGEEKP | | | mutation; |
| | | | | | L42 to V |
| | | | | | mutation |
| 51 | h IL-2Ralpha | ELCLYDPPEIPHATFKAMAYKEGTILNCECKRGFRRIKSGSVYMLCTGNSSH | human | block- | D4 to L |
| | (1-178) | SSWDNQCQCTSSATRNTTKQVTPQPEEQKERKTTEMQSPMQPVDQASLPGHC | | er | mutation; D5 |
| | D4L/D5Y/M2 | REPPPWENEATERIYHFVVGQMVYYQCVQGYRALHRGPAESVCKMTHGKTRW | | | to Y |
| | 51/ L42V | TQPQLICTGEMETSQFPGEEKP | | | mutation; |
| | | | | | M25 to I |
| | | | | | mutation; |
| | | | | | L42 to V |
| | | | | | mutation |
| 52-69 | Not Used | | | | |

Blockers: IL-10R sequences

| | | | | | |
|---|---|---|---|---|---|
| 1011 | IL-10R beta | MVPPPENVRMNSVNFKNILQWESPAFAKGNLTFTAQYLSYRIFQDKCMNTTL | human | block- | wild-type |
| | | TECDFSSLSKYGDHTLRVRAEFADEHSDWVNITFCPVDDTIIGPPGMQVEVL | | er | ECD |
| | | ADSLHMRFLAPKIENEYETWTMKNVYNSWTYNVQYWKNGTDEKFQITPQYDF | | | |
| | | EVLRNLEPWTTYCVQVRGFLPDRNKAGEWSEPVCEQTTHDETVPS | | | |
| 1012 | IL-10R alpha | HGTELPSPPSVWFEAEFFHHILHWTPIPNQSESTCYEVALLRYGIESWNSIS | human | block- | wild-type |
| | | NCSQTLSYDLTAVTLDLYHSNGYRARVRAVDGSRHSNWTVTNTRFSVDEVTL | | er | ECD |
| | | TVGSVNLEIHNGFILGKIQLPRPKMAPANDTYESIFSHFREYEIAIRKVPGN | | | |
| | | FTFTHKKVKHENFSLLTSGEVGEFCVQVKPSVASRSNKGMWSKEECISLTRQ | | | |
| | | YFTVTN | | | |

Enhancers: IL-15R sequences

| | | | | | |
|---|---|---|---|---|---|
| 1013 | IL-15R alpha | ITCPPPMSVEHADIWVKSYSLYSRERYICNSGFKRKAGTSSLTECVLNKATN | human | cytokine | wild-type |
| | (1-175) | VAHWTTPSLKCIRDPALVHQRPAPPSTVTTAGVTPQPESLSPSGKEPAASSP | | enhancer | ECD |
| | | SSNNTAATTAAIVPGSQLMPSKSPSTGTTEISSHESSHGTPSQTTAKNWELT | | | |
| | | ASASHQPPGVYPQGHSDTT | | | |
| 1014 | IL-15R alpha | ITCPPPMSVEHADIWVKSYSLYSRERYICNSGFKRKAGTSSLTECVLNKATN | human | cytokine | wild-type |
| | (1-170) | VAHWTTPSLKCIRDPALVHQRPAPPSTVTTAGVTPQPESLSPSGKEPAASSP | | enhancer | ECD |
| | | SSNNTAATTAAIVPGSQLMPSKSPSTGTTEISSHESSHGTPSQTTAKNWELT | | | |
| | | ASASHQPPGVYPQG | | | |
| 1015 | IL-15R alpha | ITCPPPMSVE HADIWVKSYS LYSRERYICN | human | cytokine | wild-type |
| | (1-77) | SGFKRKAGTS SLTECVLNKA TNVAHWTTPS | | enhancer | sushi |
| | | LKCIRDPALV HQRPAPP | | | domain |

Blockers: IL-15R sequences

| | | | | | |
|---|---|---|---|---|---|
| 1016 | Human | AVNGTSQFTCFYNSRANISCVWSQDGALQDTSCQVHAWPDRRRWNQTCELL | human | block- | full |
| | IL2Rbeta (1- | PVSQASWACNLILGAPDSQKLTTVDIVTLRVLCREGVRWRVMAIQDFKPFE | | er | length |
| | 214) | NLRLMAPISLQVVHVETHRCNISWEISQASHYFERHLEFEARTLSPGHTWE | | | ECD |
| | | EAPLLTLKQKQEWICLETLTPDTQYEFQVRVKPLQGEFTTWSPWSQPLAFR | | | |
| | | TKPAALGKDT | | | |
| 1017 | Human | LNTTILTPNGNEDTTADFFLTTMPTDSLSVSTLPLPEVQCFVFNVEYMNCTW | human | block- | full |
| | IL2R gamma | NSSSEPQPTNLTLHYWYKNSDNDKVQKCSHYLFSEEITSGCQLQKKEIHLYQ | | er | length |
| | (1-240) | TFVVQLQDPREPRRQATQMLKLQNLVIPWAPENLTLHKLSESQLELNWNNRF | | | ECD |
| | | LNHCLEHLVQYRTDWDHSWTEQSVDYRHKFSLPSVDGQKRYTFRVRSRFNPL | | | |
| | | CGSAQHWSEWSHPIHWGSNTSKENPFLFALEA | | | |
| 1018 | Human | AVNGTSQFTCFYNSRANISCVWSQDGALQDTSCQVHAWPDRRRWNQTCELLP | human | block- | truncated |
| | IL2Rbeta (1- | VSQASWACNLILGAPDSQKLTTVDIVTLRVLCREGVRWRVMAIQDFKPFENL | | er | ECD |
| | 162) | RLMAPISLQVVHVETHRCNISWEISQASHYFERHLEFEARTLSPGHTWEEAP | | | |
| | | LLTLKQ | | | |
| 1019 | Human | AVNGTSQFTCFYNSRANISCVWSQDGALQDTSCQVHAWPDRRRWNQTCELLP | human | block- | truncated |
| | IL2Rbeta (1- | VSQASWACNLILGAPDSQKLTTVDIVTLRVLCREGVRWRVMAIQDFKPFENL | | er | ECD |
| | 120) | RLMAPISLQVVHVETH | | | |

Blockers: CXCR3 sequences

| | | | | | |
|---|---|---|---|---|---|
| 1020 | CXCR3 | MVLEVSDHQVLNDAEVAALLENFSSSYDYGENESDSCCTSPPCPQDFSLNFD | human | block- | wild-type |
| | | RAFLPALYSLLFLLGLLGNGAVAAVLLSRRTALSSTDTFLLHLAVADTLLVL | | er | |
| | | TLPLWAVDAAVQWVFGSGLCKVAGALFNINFYAGALLLACISFDRYLNIVHA | | | |
| | | TQLYRRGPPARVTLTCLAVWGLCLLFALPDFIFLSAHHDERLNATHCQYNFP | | | |
| | | QVGRTALRVLQLVAGFLLPLLVMAYCYAHILAVLLVSRGQRRLRAMRLVVV | | | |
| | | VVVAFALCWTPYHLVVLVDILMDLGALARNCGRESRVDVAKSVTSGLGYMHC | | | |
| | | CLNPLLYAFVGVKFRERMWMLLLRLGCPNQRGLQRQPSSSRRDSSWSETSEA | | | |
| | | SYSGL | | | |
| 1021 | CXCR3 (22-42) | NFSSSYDYGENESDSSSTSPP | human | block- | N-term |
| | | | | er | fragment |

Blockers: TGF-βR sequences

| | | | | | |
|---|---|---|---|---|---|
| 1022 | m TGFb R II | IPPHVPK SDVEMEAQKD ASIHLSCNRT | mouse | TGFb | wild-type |
| | (1-161) | IHPLKHFNSD VMASDNGGAV KLPQLCKFCD | | trap | ECD |
| | | VRLSTCDNQK SCMSNCSITA ICEKPHEVCV | | | domain |
| | | AVWRKNDKNI TLETVCHDPK LTYHGFTLED | | | of ligand |
| | | AASPKCVMKE KKRAGETFFM CACNMEECND | | | receptor |
| | | YIIFSEEYTT SSPD | | | |
| 1023 | hu TGFb R II | TIPPHVQK SVNNDMIVTD NNGAVKFPQL | human | TGFb | wild-type |
| | (1-136) | CKFCDVRFST CDNQKSCMSN CSITSICEKP | | trap | ECD |
| | of ligand | QEVCVAVWRK NDENITLETV CHDPKLPYHD | | | domain |
| | receptor | FILEDAASPK CIMKEKKKPG ETFFMCSCSS | | | |
| | | DECNDNIIFS EEYNTSNPD | | | |

Pharmacokinetic modulators

| | | | | | |
|---|---|---|---|---|---|
| 70 | h IgG1 Fc | DKTHTCPPCPAPELLGGPSVFLFPPKPKDTLMISRTPEVTCVVVDVSHEDPE | human | half-life | C-terminal |
| | | VKFNWYVDGVEVHNAKTKPREEQYNSTYRVVSVLTVLHQDWLNGKEYKCKVS | | extension | K residue |
| | | NKALPAPIEKTISKAKGQPREPQVYTLPPSRDELTKNQVSLTCLVKGFYPSD | | | deleted |
| | | IAVEWESNGQPENNYKTTPPVLDSDGSFFLYSKLTVDKSRWQQGNVFSCSVM | | | |
| | | HEALHNHYTQKSLSLSPG | | | |
| 71 | Human IgG1 | DKTHTCPPCPAPELLGGPSVFLFPPKPKDTLMISRTPEVTCVVVDVSHEDPE | human | half-life | |
| | K392D | VKFNWYVDGVEVHNAKTKPREEQYNSTYRVVSVLTVLHQDWLNGKEYKCKVS | | extension | |
| | K409D Fc | NKALPAPIEKTISKAKGQPREPQVYTLPPSRDELTKNQVSLTCLVKGFYPSD | | | |
| | domain | IAVEWESNGQPENNYDTTPPVLDSDGSFFLYSDLTVDKSRWQQGNVFSCSVM | | | |
| | polypeptide | HEALHNHYTQKSLSLSPG | | | |
| | sequence | | | | |
| 72 | Human serum | RGVFRRDAHKSEVAHRFKDLGEENFKALVLIA | human | half-life | wild-type |
| | albumin | FAQYLQQCPFEDHVKLVNEVTEFAKTCVADESAENCDKSLHTLFGDKLCT | | extension | |
| | | VATLRETYGEMADCCAKQEPERNECFLQHKDDNPNLPRLVRPEVDVMCTA | | | |
| | | FHDNEETFLKKYLYEIARRHPYFYAPELLFFAKRYKAAFTECCQAADKAAC | | | |
| | | LLPKLDELRDEGKASSAKQRLKCASLQKFGERAFKAWAVARLSQRFPKA | | | |
| | | EFAEVSKLVTDLTKVHTECCHGDLLECADDRADLAKYICENQDSISSKLK | | | |
| | | ECCEKPLLEKSHCIAEVENDEMPADLPSLAADFVESKDVCKNYAEAKDVF | | | |
| | | LGMFLYEYARRHPDYSVVLLLRLAKTYETTLEKCCAAADPHECYAKVFDE | | | |
| | | FKPLVEEPQNLIKQNCELFEQLGEYKFQNALLVRYTKKVPQVSTPTLVEV | | | |
| | | SRNLGKVGSKCCKHPEAKRMPCAEDYLSVVLNQLCVLHEKTPVSDRVTKC | | | |
| | | CTESLVNRRPCFSALEVDETYVPKEFNAETFTFHADICTLSEKERQIKKQ | | | |
| | | TALVELVKHKPKATKEQLKAVMDDFAAFVEKCCKADDKETCFAEEGKKLV | | | |
| | | AASQAALGL | | | |
| 73 | m IgG1 Fc | GCKPCICTVPEVSSVFIFPPKPKDVLTITLTPKVTCVVVDISKDDPEVQFSW | mouse | half-life | wild-type |
| | | FVDDVEVHTAQTQPREEQFNSTFRSVSELPIMHQDWLNGKEFKCRVNSAAFP | | extension | |
| | | APIEKTISKTKGRPKAPQVYTIPPPKEQMAKDKVSLTCMITDFFPEDITVEW | | | |
| | | QWNGQPAENYKNTQPIMDTDGSYFVYSKLNVQKSNWEAGNTFTCSVLHEGLH | | | |
| | | NHHTEKSLSHSPGK | | | |
| 74 | Murine IgG1 | GCKPCICTVPEVSSVFIFPPKPKDVLMITLTPKVTCVVVDISKDDPEVQFSW | mouse | half-life | |
| | T252M Fc | FVDDVEVHTAQTQPREEQFNSTFRSVSELPIMHQDWLNGKEFKCRVNSAAFP | | extension | |
| | domain | APIEKTISKTKGRPKAPQVYTIPPPKEQMAKDKVSLTCMITDFFPEDITVEW | | | |
| | polypeptide | QWNGQPAENYKNTQPIMDTDGSYFVYSKLNVQKSNWEAGNTFTCSVLHEGLH | | | |
| | sequence | NHHTEKSLSHSPG | | | |
| 75 | hIgG1 Fc | EPKSSDKTHTCPPCPAPEAAGGPSVFLFPPKPKDTLMISRTPEVTCVVVDVS | human | PK | hetero- |
| | knob | HEDPEVKFNWYVDGVEVHNAKTKPREEQYNSTYRVVSVLTVLHQDWLNGKEY | | extender | dimeric |
| | L234A/L235 | KCKVSNKALPAPIEKTISKAKGQPREPQVYTLPPSREEMTKNQVSLWCLVKG | | | Fc fusion |
| | A | FYPSDIAVEWESNGQPENNYKTTPPVLDSDGSFFLYSKLTVDKSRWQQGNVF | | | arm 1 FcgR |
| | | SCSVMHEALHNHYTQKSLSLSPG | | | binding |
| | | | | | deficient |
| 76 | hIgG1 Fc | EPKSSDKTHTCPPCPAPEAAGGPSVFLFPPKPKDTLMISRTPEVTCVVVDVS | human | PK | hetero- |
| | knob | HEDPEVKFNWYVDGVEVHNAKTKPREEQYNSTYRVVSVLTVLHQDWLNGKEY | | extender | dimeric |
| | (L234A/L235 | KCKVSNKALPAPIEKTISKAKGQPREPQVYTLPPSREEMTKNQVSLWCLVKG | | | Fc fusion |
| | A, H435R, | FYPSDIAVEWESNGQPENNYKTTPPVLDSDGSFFLYSKLTVDKSRWQQGNVF | | | arm 1 FcgR |
| | Y436F) | SCSVMHEALHNRFTQKSLSLSPG | | | binding |
| | | | | | deficient / |
| | | | | | protein A |
| | | | | | binding |
| | | | | | deficient |
| 77 | hIgG1 Fc hole | EPKSSDKTHTCPPCPAPEAAGGPSVFLFPPKPKDTLMISRTPEVTCVVVDVS | human | PK | hetero- |
| | L234A/L235 | HEDPEVKFNWYVDGVEVHNAKTKPREEQYNSTYRVVSVLTVLHQDWLNGKEY | | extender | dimeric |
| | A | KCKVSNKALPAPIEKTISKAKGQPREPQVYTLPPSREEMTKNQVSLSCAVKG | | | Fc fusion |
| | | FYPSDIAVEWESNGQPENNYKTTPPVLDSDGSFFLVSKLTVDKSRWQQGNVF | | | arm 2 FcgR |
| | | SCSVMHEALHNHYTQKSLSLSPG | | | binding |
| | | | | | deficient |
| 78 | hIgG1 Fc hole | EPKSSDKTHTCPPCPAPEAAGGPSVFLFPPKPKDTLMISRTPEVTCVVVDVS | | | hetero- |
| | (L234A/L235 | HEDPEVKFNWYVDGVEVHNAKTKPREEQYNSTYRVVSVLTVLHQDWLNGKEY | | | dimeric |
| | | KCKVSNKALPAPIEKTISKAKGQPREPQVYTLPPSREEMTKNQVSLSCAVKG | | | Fc fusion |
| | A, H435R, | FYPSDIAVEWESNGQPENNYKTTPPVLDSDGSFFLVSKLTVDKSRWQQGNVF | | | arm 2 FcgR |
| | Y436F) | SCSVMHEALHNRFTQKSLSLSPG | | | binding |
| | | | | | deficient/ |
| | | | | | protein A |
| | | | | | binding |
| | | | | | deficient |
| 79 | Not Used | | | | |
| 756 | IgG1 Fc | DKTHTCPPCPAPELLGGPSVFLFPPKPKDTLMISRTPEVTCVVVDVSHEDPE | human | half-life | Knob |
| | (K360E/K409 | VKFNWYVDGVEVHNAKTKPREEQYNSTYRVVSVLTVLHQDWLNGKEYKCKVS | | extension | mutations |
| | W) Knob | NKALPAPIEKTISKAKGQPREPQVYTLPPSRDELTENQVSLTCLVKGFYPSD | | | |
| | | IAVEWESNGQPENNYKTTPPVLDSDGSFFLYSWLTVDKSRWQQGNVFSCSVM | | | |
| | | HEALHNHYTQKSLSLSPG | | | |
| 757 | h IgG1 Fc | DKTHTCPPCPAPELLGGPSVFLFPPKPKDTLMISRTPEVTCVVVDVSHEDPE | human | half-life | Hole |
| | (Q347R/D399 | VKFNWYVDGVEVHNAKTKPREEQYNSTYRVVSVLTVLHQDWLNGKEYKCKVS | | extension | mutations |
| | V/F405T) | NKALPAPIEKTISKAKGQPREPRVYTLPPSRDELTKNQVSLTCLVKGFYPSD | | | |
| | Hole | IAVEWESNGQPENNYKTTPPVLVSDGSFTLYSKLTVDKSRWQQGNVFSCSVM | | | |
| | | HEALHNHYTQKSLSLSPG | | | |
| 857 | h IgG1 Fc | DKTHTCPPCPAPEAAGGPSVFLFPPKPKDTLMISRTPEVTCVVVDVSHEDPE | human | PK | FcgR and |
| | (L234A/L235 | VKFNWYVDGVEVHNAKTKPREEQYNSTYRVVSVLTVLHQDWLNGKEYKCKVS | | extender | C1q binding |
| | A/P329G) | NKALGAPIEKTISKAKGQPREPQVYTLPPSRDELTKNQVSLTCLVKGFYPSD | | | impaired, |
| | | IAVEWESNGQPENNYKTTPPVLDSDGSFFLYSKLTVDKSRWQQGNVFSCSVM | | | effectorless |
| | | HEALHNHYTQKSLSLSPG | | | |
| 858 | Human IgG1 | DKTHTCPPCPAPELLGGPSVFLFPPKPKDTLMISRTPEVTCVVVDVSHEDPE | human | PK | hetero- |
| | Fc (D356K; | VKFNWYVDGVEVHNAKTKPREEQYNSTYRVVSVLTVLHQDWLNGKEYKCKVS | | extender | dimeric |
| | D399K) | NKALPAPIEKTISKAKGQPREPQVYTLPPSRKELTKNQVSLTCLVKGFYPSD | | | Fc fusion |
| | charge variant | IAVEWESNGQPENNYKTTPPVLKSDGSFFLYSKLTVDKSRWQQGNVFSCSVM | | | arm2 |
| | 2 | HEALHNHYTQKSLSLSPG | | | |
| 859 | h IgG1 Fc | DKTHTCPPCPAPEAAGGPSVFLFPPKPKDTLMISRTPEVTCV | human | PK | hetero- |
| | (L234A/L235 | VVDVSHEDPEVKFNWYVDGVEVHNAKTKPREEQYNSTYRVVSVLTVLHQD | | extender | dimeric |
| | A/P329G/K36 | WLNGKEYKCKVSNKALGAPIEKTISKAKGQPREPQVYTLPPSRDELTENQ | | | Fc fusion |
| | 0E/K409W) | VSLTCLVKGFYPSDIAVEWESNGQPENNYKTTPPVLDSDGSFFLYSWLTV | | | arm 1 FcgR |
| | Knob | DKSRWQQGNVFSCSVMHEALHNHYTQKSLSLSPG | | | and C1q |
| | | | | | binding |
| | | | | | impaired |
| 860 | h IgG1 Fc | DKTHTCPPCPAPEAAGGPSVFLFPPKPKDTLMISRTPEVTCVVVDVSHEDPE | human | PK | hetero- |
| | (L234A/L235 | VKFNWYVDGVEVHNAKTKPREEQYNSTYRVVSVLTVLHQDWLNGKEYKCKV | | extender | dimeric |
| | A/P329G/Q34 | SNKALGAPIEKTISKAKGQPREPRVYTLPPSRDELTKNQVSLTCLVKGFY | | | Fc fusion |
| | 7R/D399V/F4 | PSDIAVEWESNGQPENNYKTTPPVLVSDGSFTLYSKLTVDKSRWQQGNVF | | | arm2 FcgR |
| | 05T) Hole | SCSVMHEALHNHYTQKSLSLSPG | | | and C1q |
| | | | | | binding |
| | | | | | impaired |
| 861 | Human IgG1 | DKTHTCPPCPAPELLGGPSVFLFPPKPKDTLMISRTPEVTCVVVDVSHEDPE | human | PK | hetero- |
| | (L234A/L235 | VKFNWYVDGVEVHNAKTKPREEQYNSTYRVVSVLTVLHQDWLNGKEYKCKVS | | extender | dimeric |
| | A/P329G/K39 | NKALGAPIEKTISKAKGQPREPQVYTLPPSRDELTKNQVSLTCLVKGFYPSD | | | Fc fusion |
| | 2D/K409D) | IAVEWESNGQPENNYDTTPPVLDSDGSFFLYSDLTVDKSRWQQGNVFSCSVM | | | arm 1 FcgR |
| | Fc charge | HEALHNHYTQKSLSLSPG | | | and C1q |
| | variant 1 | | | | binding |
| | | | | | impaired |
| 862 | Human IgG1 | DKTHTCPPCPAPELLGGPSVFLFPPKPKDTLMISRTPEVTCVVVDVSHEDPE | human | PK | hetero- |
| | Fc | VKFNWYVDGVEVHNAKTKPREEQYNSTYRVVSVLTVLHQDWLNGKEYKCKVS | | extender | dimeric |
| | (L234A/L235 | NKALGAPIEKTISKAKGQPREPQVYTLPPSRKELTKNQVSLTCLVKGFYPSD | | | Fc fusion |
| | A/P329G/D35 | IAVEWESNGQPENNYKTTPPVLKSDGSFFLYSKLTVDKSRWQQGNVFSCSVM | | | arm2 FcgR |
| | 6K/ D399K) | HEALHNHYTQKSLSLSPG | | | and C1q |
| | charge variant | | | | binding |
| | 2 | | | | impaired |
| 871-899 | Not Used | | | | |

MMP cleavable segments

| | | |
|---|---|---|
| 80 | MMP | GPLGVRG |
| | cleavage site | |
| | polypeptide | |
| | sequence | |
| 81 | G112631 | GPLGVRG |
| | polypeptide | |
| | sequence | |
| 82 | G112632 | GPLGLRG |
| | polypeptide | |
| | sequence | |
| 83 | G112633 | GPLGLAR |
| | polypeptide | |
| | sequence | |
| 84 | G112634 | GPAALVGA |
| | polypeptide | |
| | sequence | |
| 85 | G112635 | GPAALIGG |
| | polypeptide | |
| | sequence | |
| 86 | G112636 | GPLNLVGR |
| | polypeptide | |
| | sequence | |
| 87 | G112637 | GPAGLVAD |
| | polypeptide | |
| | sequence | |
| 88 | G112638 | GPANLVAP |
| | polypeptide | |
| | sequence | |
| 89 | G112639 | VPLSLYSG |
| | polypeptide | |
| | sequence | |
| 90 | G112640 | SGESPAYYTA |
| | polypeptide | |
| | sequence | |
| 91 | MMP | PXXX_{HY} |
| | consensus | |
| | motif | |
| 92 | MMP-2 | (L/I)×XX_{HY} |
| | consensus | |
| | motif | |
| 93 | MMP-2 | XHYSXL |
| | consensus | |
| | motif | |
| 94 | MMP-2 | HXXX_{HY} |
| | consensus | |
| | motif | |
| 95-119 | Not Used | |
| Other | | |
| 120 | Gly-Ser rich | SGGGGSGGGG |
| | linker | |
| | polypeptide | |
| | sequence | |
| 121-178 | Not Used | |
| 179-700 | See Table 2 | |

Additional Protease-cleavable sequences

| | | |
|---|---|---|
| SEQ | Cleavable by | Sequence |
| ID | | |
| NO | | |
| 701 | MMP7 | KRALGLPG |
| 702 | MMP7 | (DE)₈RPLALWRS(DR)₈ |
| 703 | MMP9 | PR(S/T)(L/I)(S/T) |
| 704 | MMP9 | LEATA |
| 705 | MMP11 | GGAANLVRGG |
| 706 | MMP14 | SGRIGFLRTA |
| 707 | MMP | PLGLAG |
| 708 | MMP | PLGLAX |
| 709 | MMP | PLGC(me) AG |
| 710 | MMP | ESPAYYTA |
| 711 | MMP | RLQLKL |
| 712 | MMP | RLQLKAC |
| 713 | MMP, | EP(Cit)G(Hof)YL |
| | MMP9, | |
| | MMP14 | |
| 714 | Urokinase | SGRSA |
| | plasminogen | |
| | activator | |
| | (uPA) | |
| 715 | Urokinase | DAFK |
| | plasminogen | |
| | activator | |
| | (uPA) | |
| 716 | Urokinase | GGGRR |
| | plasminogen | |
| | activator | |
| | (uPA) | |
| 717 | Lysomal | GFLG |
| | Enzyme | |
| 718 | Lysomal | ALAL |
| | Enzyme | |
| 719 | Lysomal | FK |
| | Enzyme | |
| 720 | Cathepsin B | NLL |
| 721 | Cathepsin D | PIC(Et) FF |
| 722 | Cathepsin K | GGPRGLPG |
| 723 | Prostate | HSSKLQ |
| | Specific | |
| | Antigen | |
| 724 | Prostate | HSSKLQL |
| | Specific | |
| | Antigen | |
| 725 | Prostate | HSSKLQEDA |
| | Specific | |
| | Antigen | |
| 726 | Herpes | LVLASSSFGY |
| | Simplex Virus | |
| | Protease | |
| 727 | HIV Protease | GVSQNYPIVG |
| 728 | CMV | GVVQASCRLA |
| | Protease | |
| 729 | Thrombin | F(Pip)RS |
| 730 | Thrombin | DPRSFL |
| 731 | Thrombin | PPRSFL |
| 732 | Caspase-3 | DEVD |
| 733 | Caspase-3 | DEVDP |
| 734 | Caspase-3 | KGSGDVEG |
| 735 | Interleukin 1β | GWEHDG |
| | converting | |
| | enzyme | |
| 736 | Enterokinase | EDDDDKA |
| 737 | FAP | KQEQNPGST |
| 738 | Kallikrein 2 | GKAFRR |
| 739 | Plasmin | DAFK |
| 740 | Plasmin | DVLK |
| 741 | Plasmin | DAFK |
| 742 | TOP | ALLLALL |

Growth Factor-Binding and Growth Factor Receptor-Binding Sequences

| | | | | | |
|---|---|---|---|---|---|
| 760 | m TGFb R II | IPPHVPK SDVEMEAQKD ASIHLSCNRT | murine | TGFβ | wild-type |
| | (1-161) | IHPLKHFNSD VMASDNGGAV KLPQLCKFCD VRLSTCDNQK | | trap | ECD |
| | | SCMSNCSITA | | | domain |
| | | ICEKPHEVCV AVWRKNDKNI TLETVCHDPK LTYHGFTLED | | | of ligand |
| | | AASPKCVMKE | | | receptor |
| | | KKRAGETFFM CACNMEECND YIIFSEEYTT SSPD | | | |
| 761 | hu TGFb R II | TIPPHVQK SVNNDMIVTD NNGA VKFPQL CKFCDVREST | human | TGFβ | wild-type |
| | (1-136) | CDNQKSCMSN CSITSICEKP QEVCVAVWRK NDENITLETV | | trap | ECD |
| | | CHDPKLPYHD FILEDAASPK CIMKEKKKPG ETFFMCSCSS | | | domain |
| | | DECNDNIIFS EEYNTSNPD | | | of ligand |
| | | | | | receptor |
| 773 | Anti-TGFβRII | QLQVQESGPGLVKPSETLSLTCTVSGGSISNSYFSWGWIRQPPGKGLEWIGS | human | TGFβRII | antibody |
| | VH sequence | FYYGEKTYYNPSLKSRATISIDTSKSQFSLKLSSVTAADTAVYYCPRGPTMI | | antagonist | fragment |
| | | RGVIDSWGQGTLVTVSS | | | to |
| | | | | | TGFbeta |
| | | | | | Receptor |
| | | | | | II |
| 774 | Anti-TGFβRII | EIVLTQSPATLSLSPGERATLSCRASQSVRSYLAWYQQKPGQAPRLLIYDAS | human | TGFβRII | antibody |
| | VL sequence | NRATGIPARFSGSGSGTDFTLTISSLEPEDFAVYYCQQRSNWPPTFGQGTKV | | antagonist | fragment |
| | | EIK | | | to |
| | | | | | TGFbeta |
| | | | | | Receptor |
| | | | | | II |
| 775 | Anti-TGFβRII | QLQVQESGPGLVKPSETLSLTCTVSGGSISNSYFSWGWIRQPPGKGLEWIGS | human | TGFβRII | antibody |
| | scFv sequence | FYYGEKTYYNPSLKSRATISIDTSKSQFSLKLSSVTAADTAVYYCPRGPTMI | | antagonist | fragment |
| | | RGVIDSWGQGTLVTVSSGGGGSGGGGSGGGGSEIVLTQSPATLSLSPGERAT | | | to |
| | | LSCRASQSVRSYLAWYQQKPGQAPRLLIYDASNRATGIPARFSGSGSGTDFT | | | TGFbeta |
| | | LTISSLEPEDFAVYYCQQRSNWPPTFGQGTKVEIK | | | Receptor |
| | | | | | II |
| 776 | Anti-TGFβRII | EIVLTQSPATLSLSPGERATLSCRASQSVRSYLAWYQQKPGQAPRLLIYDAS | human | TGFβRII | antibody |
| | scFv sequence | NRATGIPARFSGSGSGTDFTLTISSLEPEDFAVYYCQQRSNWPPTFGQGTKV | | antagonist | fragment |
| | | EIKGGGGSGGGGSGGGGSQLQVQESGPGLVKPSETLSLTCTVSGGSISNSYF | | | to |
| | | SWGWIRQPPGKGLEWIGSFYYGEKTYYNPSLKSRATISIDTSKSQFSLKLSS | | | TGFbeta |
| | | VTAADTAVYYCPRGPTMIRGVIDSWGQGTLVTVSS | | | Receptor |
| | | | | | II |
| 777 | Anti-TGFβRII | QLQLQESGPGLVKPSETLSLTCTVSGGSISSSSYSWGWIRQPPGKGLEWIGS | human | TGFβRII | antibody |
| | VH sequence | FYYSGITYYSPSLKSRIIISEDTSKNQFSLKLSSVTAADTAVYYCASGFTMI | | antagonist | fragment |
| | | RGALDYWGQGTLVTVSS | | | to |
| | | | | | TGFbeta |
| | | | | | Receptor |
| | | | | | II |
| 778 | Anti-TGFβRII | EIVLTQSPATLSLSPGERATLSCRASQSVRSFLAWYQQKPGQAPRLLIYDAS | human | TGFβRII | antibody |
| | VL sequence | NRATGIPARFSGSGSGTDFTLTISSLEPEDFAVYYCQQRSNWPPTFGQGTKV | | antagonist | fragment |
| | | EIK | | | to |
| | | | | | TGFbeta |
| | | | | | Receptor |
| | | | | | II |
| 779 | Anti-TGFβRII | QLQLQESGPGLVKPSETLSLTCTVSGGSISSSSYSWGWIRQPPGKGLEWIGS | human | TGFβRII | antibody |
| | scFv sequence | FYYSGITYYSPSLKSRIIISEDTSKNQFSLKLSSVTAADTAVYYCASGFTMI | | antagonist | fragment |
| | | RGALDYWGQGTLVTVSSGGGGSGGGGSGGGGSEIVLTQSPATLSLSPGERAT | | | to |
| | | LSCRASQSVRSFLAWYQQKPGQAPRLLIYDASNRATGIPARFSGSGSGTDFT | | | TGFbeta |
| | | LTISSLEPEDFAVYYCQQRSNWPPTFGQGTKVEIK | | | Receptor |
| | | | | | II |
| 780 | Anti-TGFβRII | EIVLTQSPATLSLSPGERATLSCRASQSVRSFLAWYQQKPGQAPRLLIYDAS | human | TGFβRII | antibody |
| | scFv sequence | NRATGIPARFSGSGSGTDFTLTISSLEPEDFAVYYCQQRSNWPPTFGQGTKV | | antagonist | fragment |
| | | EIKGGGGSGGGGSGGGGSQLQLQESGPGLVKPSETLSLTCTVSGGSISSSSY | | | to |
| | | SWGWIRQPPGKGLEWIGSFYYSGITYYSPSLKSRIIISEDTSKNQFSLKLSS | | | TGFbeta |
| | | VTAADTAVYYCASGFTMIRGALDYWGQGTLVTVSS | | | Receptor |
| | | | | | II |

Pro-cytokine polypeptides

| | | | |
|---|---|---|---|
| 800 | Construct B | APTSSSTSSSTAEAQQQQQQQQQQQQHLEQLLMDLQELLSRMENYRNLKLPR | |
| | polypeptide | MLTFKFYLPKQATELKDLQCLEDELGPLRHVLDLTQSKSFQLEDAENFISNI | |
| | sequence: m | RVTVVKLKGSDNTFECQFDDESATVVDFLRRWIAFCQSIISTSPQSGGGGSG | |
| | IL2- | GGGGPLGVRGGGGGSGGGGSELCLYDPPEVPNATFKALSYKNGTILNCECKR | |
| | 2×(SG4)(SEQ | GFRRLKELVYMRCLGNSWSSNCQCTSNSHDKSRKQVTAQLEHQKEQQTTTDM | |
| | ID NO: 1143) | QKPTQSMHQENLTGHCREPPPWKHEDSKRIYHFVEGQSVHYECIPGYKALQR | |
| | -MMPcs1- | GPAISICKMKCGKTGWTQPQLTCVDEREHHRFLASEESQGSRNSSPESETSC | |
| | 2×(G4S) | PITTTDFPQPTETTAMTETFVLTMEYKGCKPCICTVPEVSSVFIFPPKPKDV | |
| | (SEQ | LTITLTPKVTCVVVDISKDDPEVQFSWFVDDVEVHTAQTQPREEQFNSTERS | |
| | ID NO: 1142) | VSELPIMHQDWLNGKEFKCRVNSAAFPAPIEKTISKTKGRPKAPQVYTIPPP | |
| | -IL2Ralpha- | KEQMAKDKVSLTCMITDFFPEDITVEWQWNGQPAENYKNTQPIMDTDGSYFV | |
| | mIgG1 Fc | YSKLNVQKSNWEAGNTFTCSVLHEGLHNHHTEKSLSHSPGK | |
| 801 | Construct | APTSSSTSSSTAEAQQQQQQQQQQQQHLEQLLMDLQELLSRMENYRNLKLPR | |
| | GGG | MLTFKFYLPKQATELKDLQCLEDELGPLRHVLDLTQSKSFQLEDAENFISNI | |
| | | RVTVVKLKGSDNTFECQFDDESATVVDFLRRWIAFSQSIISTSPQSGGGGSG | |
| | | GGGGVRLGPGGGGGSGGGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 802 | Construct | APTSSSTKKTQLQLEHLLLDLQMILNGINNYKNPKLTRMLTFKFYMPKKATE | |
| | AAA | LKHLQCLEEELKPLEEVLNLAQSKNFHLRPRDLISNINVIVLELKGSETTFM | |
| | | CEYADETATIVEFLNRWITFSQSIISTLTSGGGGSGGGGGPLGVRGGGGGSG | |
| | | GGGSELCDDDPPEIPHATFKAMAYKEGTILNCECKRGFRRIKSGSLYMLCTG | |
| | | NSSHSSWDNQCQCTSSATRNTTKQVTPQPEEQKERKTTEMQSPMQPVDQASL | |
| | | PGHCREPPPWENEATERIYHFVVGQMVYYQCVQGYRALHRGPAESVCKMTHG | |
| | | KTRWTQPQLICTGEMETSQFPGEEKPQASPEGRPESETSCLVTTTDFQIQTE | |
| | | MAATMETSIFTTEYQGSGGGGDKTHTCPPCPAPEAAGGPSVFLFPPKPKDTL | |
| | | MISRTPEVTCVVVDVSHEDPEVKFNWYVDGVEVHNAKTKPREEQYNSTYRVV | |
| | | SVLTVLHQDWLNGKEYKCKVSNKALPAPIEKTISKAKGQPREPQVYTLPPSR | |
| | | DELTKNQVSLTCLVKGFYPSDIAVEWESNGQPENNYKTTPPVLDSDGSFFLY | |
| | | SKLTVDKSRWQQGNVFSCSVMHEALHNHYTQKSLSLSPG | |
| 803 | Construct Y | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFSQSIISTSPQVRIQRKK | |
| | | EKMKETGPLGVRGGGGGSGGGGSELCLYDPPEVPNATFKALSYKNGTILNCE | |
| | | CKRGFRRLKELVYMRCLGNSWSSNCQCTSNSHDKSRKQVTAQLEHQKEQQTT | |
| | | TDMQKPTQSMHQENLTGHCREPPPWKHEDSKRIYHFVEGQSVHYECIPGYKA | |
| | | LQRGPAISICKMKCGKTGWTQPQLTCVDEREHHRFLASEESQGSRNSSPESE | |
| | | TSCPITTTDFPQPTETTAMTETFVLTMEYKIEGRMDGCKPCICTVPEVSSVF | |
| | | IFPPKPKDVLTITLTPKVTCVVVDISKDDPEVQFSWFVDDVEVHTAQTQPRE | |
| | | EQFNSTFRSVSELPIMHQDWLNGKEFKCRVNSAAFPAPIEKTISKTKGRPKA | |
| | | PQVYTIPPPKEQMAKDKVSLTCMITDFFPEDITVEWQWNGQPAENYKNTQPI | |
| | | MDTDGSYFVYSKLNVQKSNWEAGNTFTCSVLHEGLHNHHTEKSLSHSPGK | |
| 804 | Construct AA | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGFHRR | |
| | | IKAGPLGVRGGGGGSGGGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 805 | Construct BB | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGFHRR | |
| | | IKAGVRLGPGGGGGSGGGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 806 | Construct CC | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQGHHPHGH | |
| | | HPHGPLGVRGGGGGSGGGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 807 | Construct DD | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQGHHPHGH | |
| | | HPHGVRLGPGGGGGSGGGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 808 | Construct EE | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGWS | |
| | | HWGPLGVRGGGGGSGGGGSELCLYDPPEVPNATFKALSYKNGTILNCECKRG | |
| | | FRRLKELVYMRCLGNSWSSNCQCTSNSHDKSRKQVTAQLEHQKEQQTTTDMQ | |
| | | KPTQSMHQENLTGHCREPPPWKHEDSKRIYHFVEGQSVHYECIPGYKALQRG | |
| | | PAISICKMKCGKTGWTQPQLTCVDEREHHRFLASEESQGSRNSSPESETSCP | |
| | | ITTTDFPQPTETTAMTETFVLTMEYKGCKPCICTVPEVSSVFIFPPKPKDVL | |
| | | TITLTPKVTCVVVDISKDDPEVQFSWFVDDVEVHTAQTQPREEQFNSTFRSV | |
| | | SELPIMHQDWLNGKEFKCRVNSAAFPAPIEKTISKTKGRPKAPQVYTIPPPK | |
| | | EQMAKDKVSLTCMITDFFPEDITVEWQWNGQPAENYKNTQPIMDTDGSYFVY | |
| | | SKLNVQKSNWEAGNTFTCSVLHEGLHNHHTEKSLSHSPGK | |
| 809 | Construct FF | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGWS | |
| | | HWGVRLGPGGGGGSGGGGSELCLYDPPEVPNATFKALSYKNGTILNCECKRG | |
| | | FRRLKELVYMRCLGNSWSSNCQCTSNSHDKSRKQVTAQLEHQKEQQTTTDMQ | |
| | | KPTQSMHQENLTGHCREPPPWKHEDSKRIYHFVEGQSVHYECIPGYKALQRG | |
| | | PAISICKMKCGKTGWTQPQLTCVDEREHHRFLASEESQGSRNSSPESETSCP | |
| | | ITTTDFPQPTETTAMTETFVLTMEYKGCKPCICTVPEVSSVFIFPPKPKDVL | |
| | | TITLTPKVTCVVVDISKDDPEVQFSWFVDDVEVHTAQTQPREEQFNSTFRSV | |
| | | SELPIMHQDWLNGKEFKCRVNSAAFPAPIEKTISKTKGRPKAPQVYTIPPPK | |
| | | EQMAKDKVSLTCMITDFFPEDITVEWQWNGQPAENYKNTQPIMDTDGSYFVY | |
| | | SKLNVQKSNWEAGNTFTCSVLHEGLHNHHTEKSLSHSPGK | |
| 810 | Construct GG | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGKLWV | |
| | | LPKGPLGVRGGGGGSGGGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 811 | Construct HH | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGKLWV | |
| | | LPKGVRLGPGGGGGSGGGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 812 | Construct II | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSLHERHL | |
| | | NNNGPLGVRGGGGGSGGGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 813 | Construct JJ | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSLHERHL | |
| | | NNNGVRLGPGGGGGSGGGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 814 | Construct KK | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQVRIQRKK | |
| | | EKMKETGVRLGPGGGGGSGGGGSELCLYDPPEVPNATFKALSYKNGTILNCE | |
| | | CKRGFRRLKELVYMRCLGNSWSSNCQCTSNSHDKSRKQVTAQLEHQKEQQTT | |
| | | TDMQKPTQSMHQENLTGHCREPPPWKHEDSKRIYHFVEGQSVHYECIPGYKA | |
| | | LQRGPAISICKMKCGKTGWTQPQLTCVDEREHHRFLASEESQGSRNSSPESE | |
| | | TSCPITTTDFPQPTETTAMTETFVLTMEYKGCKPCICTVPEVSSVFIFPPKP | |
| | | KDVLTITLTPKVTCVVVDISKDDPEVQFSWFVDDVEVHTAQTQPREEQFNST | |
| | | FRSVSELPIMHQDWLNGKEFKCRVNSAAFPAPIEKTISKTKGRPKAPQVYTI | |
| | | PPPKEQMAKDKVSLTCMITDFFPEDITVEWQWNGQPAENYKNTQPIMDTDGS | |
| | | YFVYSKLNVQKSNWEAGNTFTCSVLHEGLHNHHTEKSLSHSPGK | |
| 815 | Construct LL | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGSG | |
| | | GGGGPLGVRGFHRRIKAGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 816 | Construct | APTSSSTSSSTAEAQQQQQQQQQQQQHLEQLLMDLQELLSRMENYRNLKLPR | |
| | MM | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGSG | |
| | | GGGGVRLGPGFHRRIKAGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 817 | Construct NN | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGSG | |
| | | GGGGPLGVRGGHHPHGHHPHELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 818 | Construct OO | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGSG | |
| | | GGGGVRLGPGGHHPHGHHPHELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 819 | Construct PP | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGSG | |
| | | GGGGPLGVRGGGWSHWGGSELCLYDPPEVPNATFKALSYKNGTILNCECKRG | |
| | | FRRLKELVYMRCLGNSWSSNCQCTSNSHDKSRKQVTAQLEHQKEQQTTTDMQ | |
| | | KPTQSMHQENLTGHCREPPPWKHEDSKRIYHFVEGQSVHYECIPGYKALQRG | |
| | | PAISICKMKCGKTGWTQPQLTCVDEREHHRFLASEESQGSRNSSPESETSCP | |
| | | ITTTDFPQPTETTAMTETFVLTMEYKGCKPCICTVPEVSSVFIFPPKPKDVL | |
| | | TITLTPKVTCVVVDISKDDPEVQFSWFVDDVEVHTAQTQPREEQFNSTFRSV | |
| | | SELPIMHQDWLNGKEFKCRVNSAAFPAPIEKTISKTKGRPKAPQVYTIPPPK | |
| | | EQMAKDKVSLTCMITDFFPEDITVEWQWNGQPAENYKNTQPIMDTDGSYFVY | |
| | | SKLNVQKSNWEAGNTFTCSVLHEGLHNHHTEKSLSHSPGK | |
| 820 | Construct QQ | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGSG | |
| | | GGGGVRLGPGGGWSHWGGSELCLYDPPEVPNATFKALSYKNGTILNCECKRG | |
| | | FRRLKELVYMRCLGNSWSSNCQCTSNSHDKSRKQVTAQLEHQKEQQTTTDMQ | |
| | | KPTQSMHQENLTGHCREPPPWKHEDSKRIYHFVEGQSVHYECIPGYKALQRG | |
| | | PAISICKMKCGKTGWTQPQLTCVDEREHHRFLASEESQGSRNSSPESETSCP | |
| | | ITTTDFPQPTETTAMTETFVLTMEYKGCKPCICTVPEVSSVFIFPPKPKDVL | |
| | | TITLTPKVTCVVVDISKDDPEVQFSWFVDDVEVHTAQTQPREEQFNSTFRSV | |
| | | SELPIMHQDWLNGKEFKCRVNSAAFPAPIEKTISKTKGRPKAPQVYTIPPPK | |
| | | EQMAKDKVSLTCMITDFFPEDITVEWQWNGQPAENYKNTQPIMDTDGSYFVY | |
| | | SKLNVQKSNWEAGNTFTCSVLHEGLHNHHTEKSLSHSPGK | |
| 821 | Construct RR | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGSG | |
| | | GGGGPLGVRGKLWVLPKGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 822 | Construct SS | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGSG | |
| | | GGGGVRLGPGKLWVLPKGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 823 | Construct TT | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGSG | |
| | | GGGGPLGVRGLHERHLNNNGELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 824 | Construct UU | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGSG | |
| | | GGGGVRLGPGLHERHLNNNGELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 825 | Construct VV | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGGH | |
| | | HPHGPLGVRGGGGGSGGGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 826 | Construct | APTSSSTSSSTAEAQQQQQQQQQQQQHLEQLLMDLQELLSRMENYRNLKLPR | |
| | WW | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQGHHPHSG | |
| | | GGGGPLGVRGGGGGSGGGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 827 | Construct XX | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGSG | |
| | | GGGGPLGVRGGHHPHGGGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 828 | Construct YY | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGSG | |
| | | GGGGPLGVRGGGGGSGGGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGKGHHPHGHHPH | |
| 829 | Construct ZZ | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGSG | |
| | | GGGGPLGVRGGGGGSGGGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGKGHHPH | |
| 830 | Construct | APTSSSTSSSTAEAQQQQQQQQQQQQHLEQLLMDLQELLSRMENYRNLKLPR | |
| | UUU | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGSG | |
| | | GGGGPLGVRGVRIQRKKEKMKETGSELCLYDPPEVPNATFKALSYKNGTILN | |
| | | CECKRGFRRLKELVYMRCLGNSWSSNCQCTSNSHDKSRKQVTAQLEHQKEQQ | |
| | | TTTDMQKPTQSMHQENLTGHCREPPPWKHEDSKRIYHFVEGQSVHYECIPGY | |
| | | KALQRGPAISICKMKCGKTGWTQPQLTCVDEREHHRFLASEESQGSRNSSPE | |
| | | SETSCPITTTDFPQPTETTAMTETFVLTMEYKGCKPCICTVPEVSSVFIFPP | |
| | | KPKDVLTITLTPKVTCVVVDISKDDPEVQFSWFVDDVEVHTAQTQPREEQFN | |
| | | STFRSVSELPIMHQDWLNGKEFKCRVNSAAFPAPIEKTISKTKGRPKAPQVY | |
| | | TIPPPKEQMAKDKVSLTCMITDFFPEDITVEWQWNGQPAENYKNTQPIMDTD | |
| | | GSYFVYSKLNVQKSNWEAGNTFTCSVLHEGLHNHHTEKSLSHSPGK | |
| 831 | Construct | APTSSSTSSSTAEAQQQQQQQQQQQQHLEQLLMDLQELLSRMENYRNLKLPR | |
| | HHH | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGSG | |
| | | GGGGPLGVRGGGGGSGGGGSELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGKGGSGVRIQRKK | |
| | | EKMKET | |
| 832 | Construct III | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQVRIQRKK | |
| | | EKMKETGPLGVRGGGSKLWVLPKGSELCLYDPPEVPNATFKALSYKNGTILN | |
| | | CECKRGFRRLKELVYMRCLGNSWSSNCQCTSNSHDKSRKQVTAQLEHQKEQQ | |
| | | TTTDMQKPTQSMHQENLTGHCREPPPWKHEDSKRIYHFVEGQSVHYECIPGY | |
| | | KALQRGPAISICKMKCGKTGWTQPQLTCVDEREHHRFLASEESQGSRNSSPE | |
| | | SETSCPITTTDFPQPTETTAMTETFVLTMEYKGCKPCICTVPEVSSVFIFPP | |
| | | KPKDVLTITLTPKVTCVVVDISKDDPEVQFSWFVDDVEVHTAQTQPREEQFN | |
| | | STFRSVSELPIMHQDWLNGKEFKCRVNSAAFPAPIEKTISKTKGRPKAPQVY | |
| | | TIPPPKEQMAKDKVSLTCMITDFFPEDITVEWQWNGQPAENYKNTQPIMDTD | |
| | | GSYFVYSKLNVQKSNWEAGNTFTCSVLHEGLHNHHTEKSLSHSPGK | |
| 833 | Construct JJJ | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQKLWVLPK | |
| | | GGSGPLGVRGVRIQRKKEKMKETGSELCLYDPPEVPNATFKALSYKNGTILN | |
| | | CECKRGFRRLKELVYMRCLGNSWSSNCQCTSNSHDKSRKQVTAQLEHQKEQQ | |
| | | TTTDMQKPTQSMHQENLTGHCREPPPWKHEDSKRIYHFVEGQSVHYECIPGY | |
| | | KALQRGPAISICKMKCGKTGWTQPQLTCVDEREHHRFLASEESQGSRNSSPE | |
| | | SETSCPITTTDFPQPTETTAMTETFVLTMEYKGCKPCICTVPEVSSVFIFPP | |
| | | KPKDVLTITLTPKVTCVVVDISKDDPEVQFSWFVDDVEVHTAQTQPREEQFN | |
| | | STFRSVSELPIMHQDWLNGKEFKCRVNSAAFPAPIEKTISKTKGRPKAPQVY | |
| | | TIPPPKEQMAKDKVSLTCMITDFFPEDITVEWQWNGQPAENYKNTQPIMDTD | |
| | | GSYFVYSKLNVQKSNWEAGNTFTCSVLHEGLHNHHTEKSLSHSPGK | |
| 834 | Construct | APTSSSTSSSTAEAQQQQQQQQQQQQHLEQLLMDLQELLSRMENYRNLKLPR | |
| | KKK | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQTLTYTWS | |
| | | GGGSGPLGVRGGGGGSGGGGSELCLYDPPEVPNATFKALSYKNGTILNCECK | |
| | | RGFRRLKELVYMRCLGNSWSSNCQCTSNSHDKSRKQVTAQLEHQKEQQTTTD | |
| | | MQKPTQSMHQENLTGHCREPPPWKHEDSKRIYHFVEGQSVHYECIPGYKALQ | |
| | | RGPAISICKMKCGKTGWTQPQLTCVDEREHHRFLASEESQGSRNSSPESETS | |
| | | CPITTTDFPQPTETTAMTETFVLTMEYKGCKPCICTVPEVSSVFIFPPKPKD | |
| | | VLTITLTPKVTCVVVDISKDDPEVQFSWFVDDVEVHTAQTQPREEQFNSTFR | |
| | | SVSELPIMHQDWLNGKEFKCRVNSAAFPAPIEKTISKTKGRPKAPQVYTIPP | |
| | | PKEQMAKDKVSLTCMITDFFPEDITVEWQWNGQPAENYKNTQPIMDTDGSYF | |
| | | VYSKLNVQKSNWEAGNTFTCSVLHEGLHNHHTEKSLSHSPGK | |
| 835 | Construct | APTSSSTSSSTAEAQQQQQQQQQQQQHLEQLLMDLQELLSRMENYRNLKLPR | |
| | LLL | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQVRIQRKK | |
| | | EKMKETGGSGPLGVRGVRIQRKKEKMKETGSELCLYDPPEVPNATFKALSYK | |
| | | NGTILNCECKRGFRRLKELVYMRCLGNSWSSNCQCTSNSHDKSRKQVTAQLE | |
| | | HQKEQQTTTDMQKPTQSMHQENLTGHCREPPPWKHEDSKRIYHFVEGQSVHY | |
| | | ECIPGYKALQRGPAISICKMKCGKTGWTQPQLTCVDEREHHRFLASEESQGS | |
| | | RNSSPESETSCPITTTDFPQPTETTAMTETFVLTMEYKGCKPCICTVPEVSS | |
| | | VFIFPPKPKDVLTITLTPKVTCVVVDISKDDPEVQFSWFVDDVEVHTAQTQP | |
| | | REEQFNSTFRSVSELPIMHQDWLNGKEFKCRVNSAAFPAPIEKTISKTKGRP | |
| | | KAPQVYTIPPPKEQMAKDKVSLTCMITDFFPEDITVEWQWNGQPAENYKNTQ | |
| | | PIMDTDGSYFVYSKLNVQKSNWEAGNTFTCSVLHEGLHNHHTEKSLSHSPGK | |
| 836 | Construct | APTSSSTSSSTAEAQQQQQQQQQQQQHLEQLLMDLQELLSRMENYRNLKLPR | |
| | MMM | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQLHERHLN | |
| | | NNGGSGPLGVRGVRIQRKKEKMKETGSELCLYDPPEVPNATFKALSYKNGTI | |
| | | LNCECKRGFRRLKELVYMRCLGNSWSSNCQCTSNSHDKSRKQVTAQLEHQKE | |
| | | QQTTTDMQKPTQSMHQENLTGHCREPPPWKHEDSKRIYHFVEGQSVHYECIP | |
| | | GYKALQRGPAISICKMKCGKTGWTQPQLTCVDEREHHRFLASEESQGSRNSS | |
| | | PESETSCPITTTDFPQPTETTAMTETFVLTMEYKGCKPCICTVPEVSSVFIF | |
| | | PPKPKDVLTITLTPKVTCVVVDISKDDPEVQFSWFVDDVEVHTAQTQPREEQ | |
| | | FNSTFRSVSELPIMHQDWLNGKEFKCRVNSAAFPAPIEKTISKTKGRPKAPQ | |
| | | VYTIPPPKEQMAKDKVSLTCMITDFFPEDITVEWQWNGQPAENYKNTQPIMD | |
| | | TDGSYFVYSKLNVQKSNWEAGNTFTCSVLHEGLHNHHTEKSLSHSPGK | |
| 837 | Construct | APTSSSTKKTQLQLEHLLLDLQMILNGINNYKNPKLTRMLTFKFYMPKKATE | |
| | CCC | LKHLQCLEEELKPLEEVLNLAQSKNFHLRPRDLISNINVIVLELKGSETTFM | |
| | | CEYADETATIVEFLNRWITFSQSIISTLTGHHPHGHHPHGVRLGPGGGGGSG | |
| | | GGGSELCDDDPPEIPHATFKAMAYKEGTILNCECKRGFRRIKSGSLYMLCTG | |
| | | NSSHSSWDNQCQCTSSATRNTTKQVTPQPEEQKERKTTEMQSPMQPVDQASL | |
| | | PGHCREPPPWENEATERIYHFVVGQMVYYQCVQGYRALHRGPAESVCKMTHG | |
| | | KTRWTQPQLICTGEMETSQFPGEEKPQASPEGRPESETSCLVTTTDFQIQTE | |
| | | MAATMETSIFTTEYQGSGGGGDKTHTCPPCPAPEAAGGPSVFLFPPKPKDTL | |
| | | MISRTPEVTCVVVDVSHEDPEVKFNWYVDGVEVHNAKTKPREEQYNSTYRVV | |
| | | SVLTVLHQDWLNGKEYKCKVSNKALPAPIEKTISKAKGQPREPQVYTLPPSR | |
| | | DELTKNQVSLTCLVKGFYPSDIAVEWESNGQPENNYKTTPPVLDSDGSFFLY | |
| | | SKLVDKSRWQQGNVFSCSVMHEALHNHYTQKSLSLSPG | |
| 838 | Construct | APTSSSTKKTQLQLEHLLLDLQMILNGINNYKNPKLTRMLTFKFYMPKKATE | |
| | DDD | LKHLQCLEEELKPLEEVLNLAQSKNFHLRPRDLISNINVIVLELKGSETTFM | |
| | | CEYADETATIVEFLNRWITFSQSIISTLTGHHPHGHHPHGPLGVRGGGGGSG | |
| | | GGGSELCDDDPPEIPHATFKAMAYKEGTILNCECKRGFRRIKSGSLYMLCTG | |
| | | NSSHSSWDNQCQCTSSATRNTTKQVTPQPEEQKERKTTEMQSPMQPVDQASL | |
| | | PGHCREPPPWENEATERIYHFVVGQMVYYQCVQGYRALHRGPAESVCKMTHG | |
| | | KTRWTQPQLICTGEMETSQFPGEEKPQASPEGRPESETSCLVTTTDFQIQTE | |
| | | MAATMETSIFTTEYQGSGGGGDKTHTCPPCPAPEAAGGPSVFLFPPKPKDTL | |
| | | MISRTPEVTCVVVDVSHEDPEVKFNWYVDGVEVHNAKTKPREEQYNSTYRVV | |
| | | SVLTVLHQDWLNGKEYKCKVSNKALPAPIEKTISKAKGQPREPQVYTLPPSR | |
| | | DELTKNQVSLTCLVKGFYPSDIAVEWESNGQPENNYKTTPPVLDSDGSFFLY | |
| | | SKLTVDKSRWQQGNVFSCSVMHEALHNHYTQKSLSLSPG | |
| 839 | Construct | APTSSSTKKTQLQLEHLLLDLQMILNGINNYKNPKLTRMLTFKFYMPKKATE | |
| | EEE | LKHLQCLEEELKPLEEVLNLAQSKNFHLRPRDLISNINVIVLELKGSETTFM | |
| | | CEYADETATIVEFLNRWITFSQSIISTLTVRIQRKKEKMKETGPLGVRGGGG | |
| | | GSGGGGSELCDDDPPEIPHATFKAMAYKEGTILNCECKRGFRRIKSGSLYML | |
| | | CTGNSSHSSWDNQCQCTSSATRNTTKQVTPQPEEQKERKTTEMQSPMQPVDQ | |
| | | ASLPGHCREPPPWENEATERIYHFVVGQMVYYQCVQGYRALHRGPAESVCKM | |
| | | THGKTRWTQPQLICTGEMETSQFPGEEKPQASPEGRPESETSCLVTTTDFQI | |
| | | QTEMAATMETSIFTTEYQGSGGGGDKTHTCPPCPAPEAAGGPSVFLFPPKPK | |
| | | DTLMISRTPEVTCVVVDVSHEDPEVKFNWYVDGVEVHNAKTKPREEQYNSTY | |
| | | RVVSVLTVLHQDWLNGKEYKCKVSNKALPAPIEKTISKAKGQPREPQVYTLP | |
| | | PSRDELTKNQVSLTCLVKGFYPSDIAVEWESNGQPENNYKTTPPVLDSDGSF | |
| | | FLYSKLTVDKSRWQQGNVFSCSVMHEALHNHYTQKSLSLSPG | |
| 840 | Construct FFF | APTSSSTKKTQLQLEHLLLDLQMILNGINNYKNPKLTRMLTFKFYMPKKATE | |
| | | LKHLQCLEEELKPLEEVLNLAQSKNFHLRPRDLISNINVIVLELKGSETTFM | |
| | | CEYADETATIVEFLNRWITFSQSIISTLTVRIQRKKEKMKETGPLGVRGGGG | |
| | | GSGGGGSELCDDDPPEIPHATFKAMAYKEGTILNCECKRGFRRIKSGSLYML | |
| | | CTGNSSHSSWDNQCQCTSSATRNTTKQVTPQPEEQKERKTTEMQSPMQPVDQ | |
| | | ASLPGHCREPPPWENEATERIYHFVVGQMVYYQCVQGYRALHRGPAESVCKM | |
| | | THGKTRWTQPQLICTGEMETSQFPGEEKPQASPEGRPESETSCLVTTTDFQI | |
| | | QTEMAATMETSIFTTEYQGSGGGGDKTHTCPPCPAPEAAGGPSVFLFPPKPK | |
| | | DTLMISRTPEVTCVVVDVSHEDPEVKFNWYVDGVEVHNAKTKPREEQYNSTY | |
| | | RVVSVLTVLHQDWLNGKEYKCKVSNKALPAPIEKTISKAKGQPREPQVYTLP | |
| | | PSRDELTKNQVSLTCLVKGFYPSDIAVEWESNGQPENNYKTTPPVLDSDGSF | |
| | | FLYSKLTVDKSRWQQGNVFSCSVMHEALHNHYTQKSLSLSPG | |
| 841 | Construct | APTSSSTKKTQLQLEHLLLDLQMILNGINNYKNPKLTRMLTFKFYMPKKATE | |
| | NNN | LKHLQCLEEELKPLEEVLNLAQSKNFHLRPRDLISNINVIVLELKGSETTFM | |
| | | CEYADETATIVEFLNRWITFSQSIISTLTSGGKLWVLPKGPLGVRGGGGGSG | |
| | | GGGSELCDDDPPEIPHATFKAMAYKEGTILNCECKRGFRRIKSGSLYMLCTG | |
| | | NSSHSSWDNQCQCTSSATRNTTKQVTPQPEEQKERKTTEMQSPMQPVDQASL | |
| | | PGHCREPPPWENEATERIYHFVVGQMVYYQCVQGYRALHRGPAESVCKMTHG | |
| | | KTRWTQPQLICTGEMETSQFPGEEKPQASPEGRPESETSCLVTTTDFQIQTE | |
| | | MAATMETSIFTTEYQGSGGGGDKTHTCPPCPAPEAAGGPSVFLFPPKPKDTL | |
| | | MISRTPEVTCVVVDVSHEDPEVKFNWYVDGVEVHNAKTKPREEQYNSTYRVV | |
| | | SVLTVLHQDWLNGKEYKCKVSNKALPAPIEKTISKAKGQPREPQVYTLPPSR | |
| | | DELTKNQVSLTCLVKGFYPSDIAVEWESNGQPENNYKTTPPVLDSDGSFFLY | |
| | | SKLTVDKSRWQQGNVFSCSVMHEALHNHYTQKSLSLSPG | |
| 842 | Construct | APTSSSTKKTQLQLEHLLLDLQMILNGINNYKNPKLTRMLTFKFYMPKKATE | |
| | 000 | LKHLQCLEEELKPLEEVLNLAQSKNFHLRPRDLISNINVIVLELKGSETTFM | |
| | | CEYADETATIVEFLNRWITFSQSIISTLTVRIQRKKEKMKETGPLGVRGGGS | |
| | | KLWVLPKGSELCDDDPPEIPHATFKAMAYKEGTILNCECKRGFRRIKSGSLY | |
| | | MLCTGNSSHSSWDNQCQCTSSATRNTTKQVTPQPEEQKERKTTEMQSPMQPV | |
| | | DQASLPGHCREPPPWENEATERIYHFVVGQMVYYQCVQGYRALHRGPAESVC | |
| | | KMTHGKTRWTQPQLICTGEMETSQFPGEEKPQASPEGRPESETSCLVTTTDF | |
| | | QIQTEMAATMETSIFTTEYQGSGGGGDKTHTCPPCPAPEAAGGPSVFLFPPK | |
| | | PKDTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQVYT | |
| | | LPPSRDELTKNQVSLTCLVKGFYPSDIAVEWESNGQPENNYKTTPPVLDSDG | |
| | | SFFLYSKLTVDKSRWQQGNVFSCSVMHEALHNHYTQKSLSLSPG | |
| 843 | Construct PPP | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQVRIQRKK | |
| | | EKMKETGGSGPLGVRGLHERHLNNNGSELCLYDPPEVPNATFKALSYKNGTI | |
| | | LNCECKRGFRRLKELVYMRCLGNSWSSNCQCTSNSHDKSRKQVTAQLEHQKE | |
| | | QQTTTDMQKPTQSMHQENLTGHCREPPPWKHEDSKRIYHFVEGQSVHYECIP | |
| | | GYKALQRGPAISICKMKCGKTGWTQPQLTCVDEREHHRFLASEESQGSRNSS | |
| | | PESETSCPITTTDFPQPTETTAMTETFVLTMEYKGCKPCICTVPEVSSVFIF | |
| | | PPKPKDVLTITLTPKVTCVVVDISKDDPEVQFSWFVDDVEVHTAQTQPREEQ | |
| | | FNSTFRSVSELPIMHQDWLNGKEFKCRVNSAAFPAPIEKTISKTKGRPKAPQ | |
| | | VYTIPPPKEQMAKDKVSLTCMITDFFPEDITVEWQWNGQPAENYKNTQPIMD | |
| | | TDGSYFVYSKLNVQKSNWEAGNTFTCSVLHEGLHNHHTEKSLSHSPGK | |
| 844 | Construct | APTSSSTSSSTAEAQQQQQQQQQQQQHLEQLLMDLQELLSRMENYRNLKLPR | |
| | QQQ | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSLRELHL | |
| | | DNNGGPLGVRGGGGGSGGGGSELCLYDPPEVPNATFKALSYKNGTILNCECK | |
| | | RGFRRLKELVYMRCLGNSWSSNCQCTSNSHDKSRKQVTAQLEHQKEQQTTTD | |
| | | MQKPTQSMHQENLTGHCREPPPWKHEDSKRIYHFVEGQSVHYECIPGYKALQ | |
| | | RGPAISICKMKCGKTGWTQPQLTCVDEREHHRFLASEESQGSRNSSPESETS | |
| | | CPITTTDFPQPTETTAMTETFVLTMEYKGCKPCICTVPEVSSVFIFPPKPKD | |
| | | VLTITLTPKVTCVVVDISKDDPEVQFSWFVDDVEVHTAQTQPREEQFNSTFR | |
| | | SVSELPIMHQDWLNGKEFKCRVNSAAFPAPIEKTISKTKGRPKAPQVYTIPP | |
| | | PKEQMAKDKVSLTCMITDFFPEDITVEWQWNGQPAENYKNTQPIMDTDGSYF | |
| | | VYSKLNVQKSNWEAGNTFTCSVLHEGLHNHHTEKSLSHSPGK | |
| 845 | Construct | APTSSSTSSSTAEAQQQQQQQQQQQQHLEQLLMDLQELLSRMENYRNLKLPR | |
| | RRR | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQSGGGGSG | |
| | | GGGGPLGVRGLRELHLDNNGELCLYDPPEVPNATFKALSYKNGTILNCECKR | |
| | | GFRRLKELVYMRCLGNSWSSNCQCTSNSHDKSRKQVTAQLEHQKEQQTTTDM | |
| | | QKPTQSMHQENLTGHCREPPPWKHEDSKRIYHFVEGQSVHYECIPGYKALQR | |
| | | GPAISICKMKCGKTGWTQPQLTCVDEREHHRFLASEESQGSRNSSPESETSC | |
| | | PITTTDFPQPTETTAMTETFVLTMEYKGCKPCICTVPEVSSVFIFPPKPKDV | |
| | | LTITLTPKVTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQVYTIPPP | |
| | | KEQMAKDKVSLTCMITDFFPEDITVEWQWNGQPAENYKNTQPIMDTDGSYFV | |
| | | YSKLNVQKSNWEAGNTFTCSVLHEGLHNHHTEKSLSHSPGK | |
| 846 | Construct SSS | APTSSSTSSSTAEAQQQQQQQQQQQQHLEQLLMDLQELLSRMENYRNLKLPR | |
| | | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQLRELHLD | |
| | | NNGGSGPLGVRGVRIQRKKEKMKETGSELCLYDPPEVPNATFKALSYKNGTI | |
| | | LNCECKRGFRRLKELVYMRCLGNSWSSNCQCTSNSHDKSRKQVTAQLEHQKE | |
| | | QQTTTDMQKPTQSMHQENLTGHCREPPPWKHEDSKRIYHFVEGQSVHYECIP | |
| | | GYKALQRGPAISICKMKCGKTGWTQPQLTCVDEREHHRFLASEESQGSRNSS | |
| | | PESETSCPITTTDFPQPTETTAMTETFVLTMEYKGCKPCICTVPEVSSVFIF | |
| | | PPKPKDVLTITLTPKVTCVVVDISKDDPEVQFSWFVDDVEVHTAQTQPREEQ | |
| | | FNSTFRSVSELPIMHQDWLNGKEFKCRVNSAAFPAPIEKTISKTKGRPKAPQ | |
| | | VYTIPPPKEQMAKDKVSLTCMITDFFPEDITVEWQWNGQPAENYKNTQPIMD | |
| | | TDGSYFVYSKLNVQKSNWEAGNTFTCSVLHEGLHNHHTEKSLSHSPGK | |
| 847 | Construct | APTSSSTSSSTAEAQQQQQQQQQQQQHLEQLLMDLQELLSRMENYRNLKLPR | |
| | TTT | MLTFKFYLPKQATELKDLQCLEDELGPLRHVLDLTQSKSFQLEDAENFISNI | |
| | | RVTVVKLKGSDNTFECQFDDESATVVDFLRRWIAFCQSIISTSPQVRIQRKK | |
| | | EKMKETGGSGPLGVRGLRELHLDNNGSELCLYDPPEVPNATFKALSYKNGTI | |
| | | LNCECKRGFRRLKELVYMRCLGNSWSSNCQCTSNSHDKSRKQVTAQLEHQKE | |
| | | QQTTTDMQKPTQSMHQENLTGHCREPPPWKHEDSKRIYHFVEGQSVHYECIP | |
| | | GYKALQRGPAISICKMKCGKTGWTQPQLTCVDEREHHRFLASEESQGSRNSS | |
| | | PESETSCPITTTDFPQPTETTAMTETFVLTMEYKGCKPCICTVPEVSSVFIF | |
| | | PPKPKDVLTITLTPKVTCVVVDISKDDPEVQFSWFVDDVEVHTAQTQPREEQ | |
| | | FNSTFRSVSELPIMHQDWLNGKEFKCRVNSAAFPAPIEKTISKTKGRPKAPQ | |
| | | VYTIPPPKEQMAKDKVSLTCMITDFFPEDITVEWQWNGQPAENYKNTQPIMD | |
| | | TDGSYFVYSKLNVQKSNWEAGNTFTCSVLHEGLHNHHTEKSLSHSPGK | |
| 848 | Construct | ITCPPPMSVE HADIWVKSYS LYSRERYICN SGFKRKAGTS | |
| | FFFF | SLTECVLNKA TNVAHWTTPS LKCIRDPALV HQRPAPP | |
| | | SGGSGGGGSGGGSGGGGSLQ NWVNVISDLKKIE | |
| | | DLIQSMHIDATLYTESDVHPSCKVTAMKCFLLELQVISLESGDASIHDTVEN | |
| | | LIILANNSLSSNGNVTESGCKECEELEEKNIKEFLQSFVHIVQMFINTS | |
| | | HHHHHH | |
| 849 | Not Used | | |
| 1024 | Construct | ITCPPPMSVEHADIWVKSYSLYSRERYICNSGFKRKAGTSSLTECVLNKATN | |
| | VVV no TME | VAHWTTPSLKCIRDPALVHQRPAPPSGGSGGGGSGGGSGGGGSLQNWVNVIS | |
| | control | DLKKIEDLIQSMHIDATLYTESDVHPSCKVTAMKCFLLELQVISLESGDASI | |
| | | HDTVENLIILANNSLSSNGNVTESGCKECEELEEKNIKEFLQSFVHIVQMFI | |
| | | NTSSGGGGPLGVRGGGGGSGGGGSGGGGSGGGGSAVNGTSQFTCFYNSRANI | |
| | | SCVWSQDGALQDTSCQVHAWPDRRRWNQTCELLPVSQASWACNLILGAPDSQ | |
| | | KLTTVDIVTLRVLCREGVRWRVMAIQDFKPFENLRLMAPISLQVVHVETHRC | |
| | | NISWEISQASHYFERHLEFEARTLSPGHTWEEAPLLTLKQKQEWICLETLTP | |
| | | DTQYEFQVRVKPLQGEFTTWSPWSQPLAFRTKPAALGKDTHHHHHHG | |
| 1025 | Construct | ITCPPPMSVEHADIWVKSYSLYSRERYICNSGFKRKAGTSSLTECVLNKATN | |
| | WWW | VAHWTTPSLKCIRDPALVHQRPAPPSGGSGGGGSGGGSGGGGSLQNWVNVIS | |
| | | DLKKIEDLIQSMHIDATLYTESDVHPSCKVTAMKCFLLELQVISLESGDASI | |
| | | HDTVENLIILANNSLSSNGNVTESGCKECEELEEKNIKEFLQSFVHIVQMFI | |
| | | NTSSGGGGPLGVRGGSVRIQRKKEKMKETGGGGSGGGGSGGGGSAVNGTSQF | |
| | | TCFYNSRANISCVWSQDGALQDTSCQVHAWPDRRRWNQTCELLPVSQASWAC | |
| | | NLILGAPDSQKLTTVDIVTLRVLCREGVRWRVMAIQDFKPFENLRLMAPISL | |
| | | QVVHVETHRCNISWEISQASHYFERHLEFEARTLSPGHTWEEAPLLTLKQKQ | |
| | | EWICLETLTPDTQYEFQVRVKPLQGEFTTWSPWSQPLAFRTKPAALGKDTHH | |
| | | HHHHG | |
| 1026 | Construct | ITCPPPMSVEHADIWVKSYSLYSRERYICNSGFKRKAGTSSLTECVLNKATN | |
| | XXX | VAHWTTPSLKCIRDPALVHQRPAPPSGGSGGGGSGGGSGGGGSLQNWVNVIS | |
| | | DLKKIEDLIQSMHIDATLYTESDVHPSCKVTAMKCFLLELQVISLESGDASI | |
| | | HDTVENLIILANNSLSSNGNVTESGCKECEELEEKNIKEFLQSFVHIVQMFI | |
| | | NTSSGGGGPLGVRGGGSKLWVLPKGGGGGGGGSAVNGTSQFTCFYNSRANI | |
| | | SCVWSQDGALQDTSCQVHAWPDRRRWNQTCELLPVSQASWACNLILGAPDSQ | |
| | | KLTTVDIVTLRVLCREGVRWRVMAIQDFKPFENLRLMAPISLQVVHVETHRC | |
| | | NISWEISQASHYFERHLEFEARTLSPGHTWEEAPLLTLKQKQEWICLETLTP | |
| | | DTQYEFQVRVKPLQGEFTTWSPWSQPLAFRTKPAALGKDTHHHHHHG | |
| 1027 | Construct | ITCPPPMSVEHADIWVKSYSLYSRERYICNSGFKRKAGTSSLTECVLNKATN | |
| | YYY | VAHWTTPSLKCIRDPALVHQRPAPPSGGSGGGGSGGGSGGGGSLQNWVNVIS | |
| | | DLKKIEDLIQSMHIDATLYTESDVHPSCKVTAMKCFLLELQVISLESGDASI | |
| | | HDTVENLIILANNSLSSNGNVTESGCKECEELEEKNIKEFLQSFVHIVQMFI | |
| | | NTSSGGGGPLGVRGVRIQRKKEKMKETGGGGSGGGGSAVNGTSQFTCFYNSR | |
| | | ANISCVWSQDGALQDTSCQVHAWPDRRRWNQTCELLPVSQASWACNLILGAP | |
| | | DSQKLTTVDIVTLRVLCREGVRWRVMAIQDFKPFENLRLMAPISLQVVHVET | |
| | | HRCNISWEISQASHYFERHLEFEARTLSPGHTWEEAPLLTLKQKQEWICLET | |
| | | LTPDTQYEFQVRVKPLQGEFTTWSPWSQPLAFRTKPAALGKDTGGGGSGEPK | |
| | | SSDKTHTCPPCPAPEAAGGPSVFLFPPKPKDTLMISRTPEVTCVVVDVSHED | |
| | | PEVKFNWYVDGVEVHNAKTKPREEQYNSTYRVVSVLTVLHQDWLNGKEYKCK | |
| | | VSNKALPAPIEKTISKAKGQPREPQVYTLPPSRDELTKNQVSLTCLVKGFYP | |
| | | SDIAVEWESNGQPENNYKTTPPVLDSDGSFFLYSKLTVDKSRWQQGNVFSCS | |
| | | VMHEALHNHYTQKSLSLSPG | |
| 1028 | Construct | ITCPPPMSVEHADIWVKSYSLYSRERYICNSGFKRKAGTSSLTECVLNKATN | |
| | ZZZ | VAHWTTPSLKCIRDPALVHQRPAPPSGGSGGGGSGGGSGGGGSLQNWVNVIS | |
| | | DLKKIEDLIQSMHIDATLYTESDVHPSCKVTAMKCFLLELQVISLESGDASI | |
| | | HDTVENLIILANNSLSSNGNVTESGCKECEELEEKNIKEFLQSFVHIVQMFI | |
| | | NTSSGGGGPLGVRGGGSKLWVLPKGGGGSGGGGSAVNGTSQFTCFYNSRANI | |
| | | SCVWSQDGALQDTSCQVHAWPDRRRWNQTCELLPVSQASWACNLILGAPDSQ | |
| | | KLTTVDIVTLRVLCREGVRWRVMAIQDFKPFENLRLMAPISLQVVHVETHRC | |
| | | NISWEISQASHYFERHLEFEARTLSPGHTWEEAPLLTLKQKQEWICLETLTP | |
| | | DTQYEFQVRVKPLQGEFTTWSPWSQPLAFRTKPAALGKDTGGGGSGEPKSSD | |
| | | KTHTCPPCPAPEAAGGPSVFLFPPKPKDTLMISRTPEVTCVVVDVSHEDPEV | |
| | | KFNWYVDGVEVHNAKTKPREEQYNSTYRVVSVLTVLHQDWLNGKEYKCKVSN | |
| | | KALPAPIEKTISKAKGQPREPQVYTLPPSRDELTKNQVSLTCLVKGFYPSDI | |
| | | AVEWESNGQPENNYKTTPPVLDSDGSFFLYSKLTVDKSRWQQGNVFSCSVMH | |
| | | EALHNHYTQKSLSLSPG | |
| 1029 | Construct | ITCPPPMSVEHADIWVKSYSLYSRERYICNSGFKRKAGTSSLTECVLNKATN | |
| | AAAA | VAHWTTPSLKCIRDPALVHQRPAPPSGGSGGGGSGGGSGGGGSLQNWVNVIS | |
| | | DLKKIEDLIQSMHIDATLYTESDVHPSCKVTAMKCFLLELQVISLESGDASI | |
| | | HDTVENLIILANNSLSSNGNVTESGCKECEELEEKNIKEFLQSFVHIVQMFI | |
| | | NTSSGGGGPLGVRGGLRELHLDNNGGGGSGGGGSAVNGTSQFTCFYNSRANI | |
| | | SCVWSQDGALQDTSCQVHAWPDRRRWNQTCELLPVSQASWACNLILGAPDSQ | |
| | | KLTTVDIVTLRVLCREGVRWRVMAIQDFKPFENLRLMAPISLQVVHVETHRC | |
| | | NISWEISQASHYFERHLEFEARTLSPGHTWEEAPLLTLKQKQEWICLETLTP | |
| | | DTQYEFQVRVKPLQGEFTTWSPWSQPLAFRTKPAALGKDTGGGGSGEPKSSD | |
| | | KTHTCPPCPAPEAAGGPSVFLFPPKPKDTLMISRTPEVTCVVVDVSHEDPEV | |
| | | KFNWYVDGVEVHNAKTKPREEQYNSTYRVVSVLTVLHQDWLNGKEYKCKVSN | |
| | | KALPAPIEKTISKAKGQPREPQVYTLPPSRDELTKNQVSLTCLVKGFYPSDI | |
| | | AVEWESNGQPENNYKTTPPVLDSDGSFFLYSKLTVDKSRWQQGNVFSCSVMH | |
| | | EALHNHYTQKSLSLSPG | |
| 1030 | Construct | ITCPPPMSVEHADIWVKSYSLYSRERYICNSGFKRKAGTSSLTECVLNKATN | |
| | BBBB | VAHWTTPSLKCIRDPALVHQRPAPPSGGSGGGGSGGGSGGGGSLQNWVNVIS | |
| | | DLKKIEDLIQSMHIDATLYTESDVHPSCKVTAMKCFLLELQVISLESGDASI | |
| | | HDTVENLIILANNSLSSNGNVTESGCKECEELEEKNIKEFLQSFVHIVQMFI | |
| | | NTSSGGGGPLGVRGVRIQRKKEKMKETGGSKLWVLPKAVNGTSQFTCFYNSR | |
| | | ANISCVWSQDGALQDTSCQVHAWPDRRRWNQTCELLPVSQASWACNLILGAP | |
| | | DSQKLTTVDIVTLRVLCREGVRWRVMAIQDFKPFENLRLMAPISLQVVHVET | |
| | | HRCNISWEISQASHYFERHLEFEARTLSPGHTWEEAPLLTLKQKQEWICLET | |
| | | LTPDTQYEFQVRVKPLQGEFTTWSPWSQPLAFRTKPAALGKDTGGGGSGEPK | |
| | | SSDKTHTCPPCPAPEAAGGPSVFLFPPKPKDTLMISRTPEVTCVVVDVSHED | |
| | | PEVKFNWYVDGVEVHNAKTKPREEQYNSTYRVVSVLTVLHQDWLNGKEYKCK | |
| | | VSNKALPAPIEKTISKAKGQPREPQVYTLPPSRDELTKNQVSLTCLVKGFYP | |
| | | SDIAVEWESNGQPENNYKTTPPVLDSDGSFFLYSKLTVDKSRWQQGNVFSCS | |
| | | VMHEALHNHYTQKSLSLSPG | |
| 1031 | Construct | ITCPPPMSVEHADIWVKSYSLYSRERYICNSGFKRKAGTSSLTECVLNKATN | |
| | CCCC | VAHWTTPSLKCIRDPALVHQRPAPPSGGSGGGGSGGGSGGGGSLQNWVNVIS | |
| | | DLKKIEDLIQSMHIDATLYTESDVHPSCKVTAMKCFLLELQVISLESGDASI | |
| | | HDTVENLIILANNSLSSNGNVTESGCKECEELEEKNIKEFLQSFVHIVQMFI | |
| | | NTSSGGGGPLGVRGGSKLWVLPKGGSKLWVLPKGGSAVNGTSQFTCFYNSRA | |
| | | NISCVWSQDGALQDTSCQVHAWPDRRRWNQTCELLPVSQASWACNLILGAPD | |
| | | SQKLTTVDIVTLRVLCREGVRWRVMAIQDFKPFENLRLMAPISLQVVHVETH | |
| | | RCNISWEISQASHYFERHLEFEARTLSPGHTWEEAPLLTLKQKQEWICLETL | |
| | | TPDTQYEFQVRVKPLQGEFTTWSPWSQPLAFRTKPAALGKDTGGGGSGEPKS | |
| | | SDKTHTCPPCPAPEAAGGPSVFLFPPKPKDTLMISRTPEVTCVVVDVSHEDP | |
| | | EVKFNWYVDGVEVHNAKTKPREEQYNSTYRVVSVLTVLHQDWLNGKEYKCKV | |
| | | SNKALPAPIEKTISKAKGQPREPQVYTLPPSRDELTKNQVSLTCLVKGFYPS | |
| | | DIAVEWESNGQPENNYKTTPPVLDSDGSFFLYSKLTVDKSRWQQGNVFSCSV | |
| | | MHEALHNHYTQKSLSLSPG | |
| 1032 | Construct | EPKSSDKTHTCPPCPAPEAAGGPSVFLFPPKPKDTLMISRTPEVTCVVVDVS | |
| | GGGG | HEDPEVKFNWYVDGVEVHNAKTKPREEQYNSTYRVVSVLTVLHQDWLNGKEY | |
| | | KCKVSNKALPAPIEKTISKAKGQPREPQVYTLPPSRDELTKNQVSLTCLVKG | |
| | | FYPSDIAVEWESNGQPENNYKTTPPVLDSDGSFFLYSKLTVDKSRWQQGNVF | |
| | | SCSVMHEALHNHYTQKSLSLSPGVRIQRKKEKMKETGPLGVRGTPVVRKGRC | |
| | | SCISTNQGTIHLQSLKDLKQFAPSPSCEKIEIIATLKNGVQTCLNPDSADVK | |
| | | ELIKKWEKQVSQKKKQKNGKKHQKKKVLKVRKSQRSRQKKTT | |
| 1033 | Construct | EPKSSDKTHTCPPCPAPEAAGGPSVFLFPPKPKDTLMISRTPEVTCVVVDVS | |
| | HHHH | HEDPEVKFNWYVDGVEVHNAKTKPREEQYNSTYRVVSVLTVLHQDWLNGKEY | |
| | | KCKVSNKALPAPIEKTISKAKGQPREPQVYTLPPSRDELTKNQVSLTCLVKG | |
| | | FYPSDIAVEWESNGQPENNYKTTPPVLDSDGSFFLYSKLTVDKSRWQQGNVF | |
| | | SCSVMHEALHNHYTQKSLSLSPGKLWVLPKGGGPLGVRGTPVVRKGRCSCIS | |
| | | TNQGTIHLQSLKDLKQFAPSPSCEKIEIIATLKNGVQTCLNPDSADVKELIK | |
| | | KWEKQVSQKKKQKNGKKHQKKKVLKVRKSQRSRQKKTT | |
| 1034 | Construct | HHHHHHGGSGDAHKSEVAHRFKDLGEENFKALVLIAFAQYLQQCPFEDHVKL | |
| | IIII | VNEVTEFAKTCVADESAENCDKSLHTLFGDKLCTVATLRETYGEMADCCAKQ | |
| | | EPERNECFLQHKDDNPNLPRLVRPEVDVMCTAFHDNEETFLKKYLYEIARRH | |
| | | PYFYAPELLFFAKRYKAAFTECCQAADKAACLLPKLDELRDEGKASSAKQRL | |
| | | KCASLQKFGERAFKAWAVARLSQRFPKAEFAEVSKLVTDLTKVHTECCHGDL | |
| | | LECADDRADLAKYICENQDSISSKLKECCEKPLLEKSHCIAEVENDEMPADL | |
| | | PSLAADFVESKDVCKNYAEAKDVFLGMFLYEYARRHPDYSVVLLLRLAKTYE | |
| | | TTLEKCCAAADPHECYAKVFDEFKPLVEEPQNLIKQNCELFEQLGEYKFQNA | |
| | | LLVRYTKKVPQVSTPTLVEVSRNLGKVGSKCCKHPEAKRMPCAEDYLSVVLN | |
| | | QLCVLHEKTPVSDRVTKCCTESLVNRRPCFSALEVDETYVPKEFNAETFTFH | |
| | | ADICTLSEKERQIKKQTALVELVKHKPKATKEQLKAVMDDFAAFVEKCCKAD | |
| | | DKETCFAEEGKKLVAASQAALGLGGKLWVLPKGSGPLGVRGTPVVRKGRCSC | |
| | | ISTNQGTIHLQSLKDLKQFAPSPSCEKIEIIATLKNGVQTCLNPDSADVKEL | |
| | | IKKWEKQVSQKKKQKNGKKHQKKKVLKVRKSQRSRQKKTT | |
| 1035 | Construct JJJJ | HHHHHHGGSGDAHKSEVAHRFKDLGEENFKALVLIAFAQYLQQCPFEDHVKL | |
| | | VNEVTEFAKTCVADESAENCDKSLHTLFGDKLCTVATLRETYGEMADCCAKQ | |
| | | EPERNECFLQHKDDNPNLPRLVRPEVDVMCTAFHDNEETFLKKYLYEIARRH | |
| | | PYFYAPELLFFAKRYKAAFTECCQAADKAACLLPKLDELRDEGKASSAKQRL | |
| | | KCASLQKFGERAFKAWAVARLSQRFPKAEFAEVSKLVTDLTKVHTECCHGDL | |
| | | LECADDRADLAKYICENQDSISSKLKECCEKPLLEKSHCIAEVENDEMPADL | |
| | | PSLAADFVESKDVCKNYAEAKDVFLGMFLYEYARRHPDYSVVLLLRLAKTYE | |
| | | TTLEKCCAAADPHECYAKVFDEFKPLVEEPQNLIKQNCELFEQLGEYKFQNA | |
| | | LLVRYTKKVPQVSTPTLVEVSRNLGKVGSKCCKHPEAKRMPCAEDYLSVVLN | |
| | | QLCVLHEKTPVSDRVTKCCTESLVNRRPCFSALEVDETYVPKEFNAETFTFH | |
| | | ADICTLSEKERQIKKQTALVELVKHKPKATKEQLKAVMDDFAAFVEKCCKAD | |
| | | DKETCFAEEGKKLVAASQAALGLVRIQRKKEKMKETGPLGVRGTPVVRKGRC | |
| | | SCISTNQGTIHLQSLKDLKQFAPSPSCEKIEIIATLKNGVQTCLNPDSADVK | |
| | | ELIKKWEKQVSQKKKQKNGKKHQKKKVLKVRKSQRSRQKKTT | |
| 1036 | Construct | DIQMTQSPSSLSASVGDRVTITCRASQDVNTAVAWYQQKPGKAPKLLIYSAS | scFv |
| | KKKK Arm 1 | FLYSGVPSRFSGSRSGTDFTLTISSLQPEDFATYYCQQHYTTPPTFGQGTKV | Trastuzumab |
| | | EIKGGGGGGGGSGGGASEVQLVESGGGLVQPGGSLRLSCAASGFNIKDTYI | (VL-VH)-hu |
| | | HWVRQAPGKGLEWVARIYPTNGYTRYADSVKGRFTISADTSKNTAYLQMNSL | IgG1 Fc |
| | | RAEDTAVYYCSRWGGDGFYAMDYWGQGTLVTVSSEPKSSDKTHTCPPCPAPE | knob |
| | | LLGGPSVFLFPPKPKDTLMISRTPEVTCVVVDVSHEDPEVKFNWYVDGVEVH | |
| | | NAKTKPREEQYNSTYRVVSVLTVLHQDWLNGKEYKCKVSNKALPAPIEKTIS | |
| | | KAKGQPREPQVYTLPPSRDELTENQVSLTCLVKGFYPSDIAVEWESNGQPEN | |
| | | NYKTTPPVLDSDGSFFLYSWLTVDKSRWQQGNVFSCSVMHEALHNHYTQKSL | |
| | | SLSPG | |
| 1037 | Construct | APTSSSTKKTQLQLEHLLLDLQMILNGINNYKNPKLTRMLTFKFYMPKKATE | Hu linker |
| | KKKK Arm 2 | LKHLQCLEEELKPLEEVLNLAQSKNFHLRPRDLISNINVIVLELKGSETTFM | polypeptide- |
| | | CEYADETATIVEFLNRWITFSQSIISTLTSGGKLWVLPKGPLGVRGGGGGSG | IL2(TME)- |
| | | GGGSELCDDDPPEIPHATFKAMAYKEGTILNCECKRGFRRIKSGSLYMLCTG | hu IgG1 Fc |
| | | NSSHSSWDNQCQCTSSATRNTTKQVTPQPEEQKERKTTEMQSPMQPVDQASL | hole |
| | | PGHCREPPPWENEATERIYHFVVGQMVYYQCVQGYRALHRGPAESVCKMTHG | |
| | | KTRWTQPQLICTGEMETSQFPGEEKPQASPEGRPESETSCLVTTTDFQIQTE | |
| | | MAATMETSIFTTEYQGSGGGGEPKSSDKTHTCPPCPAPELLGGPSVFLFPPK | |
| | | PKDTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPRVYT | |
| | | LPPSRDELTKNQVSLTCLVKGFYPSDIAVEWESNGQPENNYKTTPPVLVSDG | |
| | | SFTLYSKLTVDKSRWQQGNVFSCSVMHEALHNHYTQKSLSLSPG | |
| 1038 | Construct | DIQMTQSPSSLSASVGDRVTITCRASQDVNTAVAWYQQKPGKAPKLLIYSAS | scFv |
| | LLLL Arm 1 | FLYSGVPSRFSGSRSGTDFTLTISSLQPEDFATYYCQQHYTTPPTFGQGTKV | Trastuzumab |
| | | EIKGGGGSGGGGSGGGASEVQLVESGGGLVQPGGSLRLSCAASGFNIKDTYI | (VL-VH)-hu |
| | | HWVRQAPGKGLEWVARIYPTNGYTRYADSVKGRFTISADTSKNTAYLQMNSL | IgG1 Fc |
| | | RAEDTAVYYCSRWGGDGFYAMDYWGQGTLVTVSSEPKSSDKTHTCPPCPAPE | knob |
| | | LLGGPSVFLFPPKPKDTLMISRTPEVTCVVVDVSHEDPEVKFNWYVDGVEVH | |
| | | NAKTKPREEQYNSTYRVVSVLTVLHQDWLNGKEYKCKVSNKALPAPIEKTIS | |
| | | KAKGQPREPQVYTLPPSRDELTENQVSLTCLVKGFYPSDIAVEWESNGQPEN | |
| | | NYKTTPPVLDSDGSFFLYSWLTVDKSRWQQGNVFSCSVMHEALHNHYTQKSL | |
| | | SLSPG | |
| 1039 | Construct | APTSSSTKKTQLQLEHLLLDLQMILNGINNYKNPKLTRMLTFKFYMPKKATE | Hu linker |
| | LLLL Arm 2 | LKHLQCLEEELKPLEEVLNLAQSKNFHLRPRDLISNINVIVLELKGSETTFM | polypeptide- |
| | | CEYADETATIVEFLNRWITFSQSIISTLTVRIQRKKEKMKETGPLGVRGGGG | IL2 (TME)- |
| | | GSGGGGSELCDDDPPEIPHATFKAMAYKEGTILNCECKRGFRRIKSGSLYML | hu IgG1 Fc |
| | | CTGNSSHSSWDNQCQCTSSATRNTTKQVTPQPEEQKERKTTEMQSPMQPVDQ | hole |
| | | ASLPGHCREPPPWENEATERIYHFVVGQMVYYQCVQGYRALHRGPAESVCKM | |
| | | THGKTRWTQPQLICTGEMETSQFPGEEKPQASPEGRPESETSCLVTTTDFQI | |
| | | QTEMAATMETSIFTTEYQGSGGGGEPKSSDKTHTCPPCPAPELLGGPSVFLF | |
| | | PPKPKDTLMISRTPEVTCVVVDVSHEDPEVKFNWYVDGVEVHNAKTKPREEQ | |
| | | YNSTYRVVSVLTVLHQDWLNGKEYKCKVSNKALPAPIEKTISKAKGQPREPR | |
| | | VYTLPPSRDELTKNQVSLTCLVKGFYPSDIAVEWESNGQPENNYKTTPPVLV | |
| | | SDGSFTLYSKLTVDKSRWQQGNVFSCSVMHEALHNHYTQKSLSLSPG | |
| 1040 | Construct | DILLTQSPVILSVSPGERVSFSCRASQSIGTNIHWYQQRTNGSPRLLIKYAS | scFv |
| | MMMM Arm | ESISGIPSRFSGSGSGTDFTLSINSVESEDIADYYCQQNNNWPTTFGAGTKL | cetuximab |
| | 1 | ELKRGGGGSGGGGSGGGASQVQLKQSGPGLVQPSQSLSITCTVSGFSLTNYG | (VL-VH)-hu |
| | | VHWVRQSPGKGLEWLGVIWSGGNTDYNTPFTSRLSINKDNSKSQVFFKMNSL | IgG1 Fc |
| | | QSNDTAIYYCARALTYYDYEFAYWGQGTLVTVSEPKSSDKTHTCPPCPAPEL | knob |
| | | LGGPSVFLFPPKPKDTLMISRTPEVTCVVVDVSHEDPEVKFNWYVDGVEVHN | |
| | | AKTKPREEQYNSTYRVVSVLTVLHQDWLNGKEYKCKVSNKALPAPIEKTISK | |
| | | AKGQPREPQVYTLPPSRDELTENQVSLTCLVKGFYPSDIAVEWESNGQPENN | |
| | | YKTTPPVLDSDGSFFLYSWLTVDKSRWQQGNVFSCSVMHEALHNHYTQKSLS | |
| | | LSPG | |
| 1041 | Construct | APTSSSTKKTQLQLEHLLLDLQMILNGINNYKNPKLTRMLTFKFYMPKKATE | Hu linker |
| | MMMM Arm | LKHLQCLEEELKPLEEVLNLAQSKNFHLRPRDLISNINVIVLELKGSETTFM | polypeptide- |
| | 2 | CEYADETATIVEFLNRWITFSQSIISTLTSGGKLWVLPKGPLGVRGGGGGSG | IL2(TME)- |
| | | GGGSELCDDDPPEIPHATFKAMAYKEGTILNCECKRGFRRIKSGSLYMLCTG | hu IgG1 Fc |
| | | NSSHSSWDNQCQCTSSATRNTTKQVTPQPEEQKERKTTEMQSPMQPVDQASL | hole |
| | | PGHCREPPPWENEATERIYHFVVGQMVYYQCVQGYRALHRGPAESVCKMTHG | |
| | | KTRWTQPQLICTGEMETSQFPGEEKPQASPEGRPESETSCLVTTTDFQIQTE | |
| | | MAATMETSIFTTEYQGSGGGGEPKSSDKTHTCPPCPAPELLGGPSVFLFPPK | |
| | | PKDTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPRVYT | |
| | | LPPSRDELTKNQVSLTCLVKGFYPSDIAVEWESNGQPENNYKTTPPVLVSDG | |
| | | SFTLYSKLTVDKSRWQQGNVFSCSVMHEALHNHYTQKSLSLSPG | |
| 1042 | Construct | APTSSSTKKTQLQLEHLLLDLQMILNGINNYKNPKLTRMLTFKFYMPKKATE | HuIL2(C125 |
| | NNNN | LKHLQCLEEELKPLEEVLNLAQSKNFHLRPRDLISNINVIVLELKGSETTFM | S)- |
| | | CEYADETATIVEFLNRWITFSQSIISTLTVRIQRKKEKMKETGPLGVRGGGG | VRIQRKKE |
| | | GSGGGGSELCDDDPPEIPHATFKAMAYKEGTILNCECKRGFRRIKSGSLYML | KMKET(SEQ |
| | | CTGNSSHSSWDNQCQCTSSATRNTTKQVTPQPEEQKERKTTEMQSPMQPVDQ | ID NO: |
| | | ASLPGHCREPPPWENEATERIYHFVVGQMVYYQCVQGYRALHRGPAESVCKM | 1139)- |
| | | THGKTRWTQPQLICTGEMETSQFPGEEKPQASPEGRPESETSCLVTTTDFQI | MMPcs1_(G |
| | | QTEMAATMETSIFTTEYQGSGGGGDKTHTCPPCPAPEAAGGPSVFLFPPKPK | 4S)(SEQ ID |
| | | DTLMISRTPEVTCVVVDVSHEDPEVKFNWYVDGVEVHNAKTKPREEQYNSTY | NO: |
| | | RVVSVLTVLHQDWLNGKEYKCKVSNKALGAPIEKTISKAKGQPREPQVYTLP | 1142)×2-hu |
| | | PSRDELTKNQVSLTCLVKGFYPSDIAVEWESNGQPENNYKTTPPVLDSDGSF | IL2Ra(1- |
| | | FLYSKLTVDKSRWQQGNVFSCSVMHEALHNHYTQKSLSLSPGGGSGKLWVLP | 219; M25I)- |
| | | K | GSGGGG |
| | | | (SEQ ID NO: |
| | | | 1138)-hu |
| | | | IgG1Fc- |
| | | | GGSGKLW |
| | | | VLPK(SEQ |
| | | | ID NO: |
| | | | 1164) |
| 1043 | Construct | APTSSSTKKTQLQLEHLLLDLQMILNGINNYKNPKLTRMLTFKFYMPKKATE | Hu |
| | SSSS | LKHLQCLEEELKPLEEVLNLAQSKNFHLRPRDLISNINVIVLELKGSETTFM | IL2(C125S)- |
| | | CEYADETATIVEFLNRWITFSQSIISTLTVRIQRKKEKMKETGPLGVRGGGG | VRIQRKKE |
| | | GSGGGGSELCDDDPPEIPHATFKAMAYKEGTILNCECKRGFRRIKSGSLYML | KMKET(SEQ |
| | | CTGNSSHSSWDNQCQCTSSATRNTTKQVTPQPEEQKERKTTEMQSPMQPVDQ | ID NO: |
| | | ASLPGHCREPPPWENEATERIYHFVVGQMVYYQCVQGYRALHRGPAESVCKM | 1139)- |
| | | THGKTRWTQPQLICTGEMETSQFPGEEKPQASPEGRPESETSCLVTTTDFQI | MMPcs1_(G |
| | | QTEMAATMETSIFTTEYQGSGGGGDKTHTCPPCPAPEAAGGPSVFLFPPKPK | 4S) (SEQ ID |
| | | DTLMISRTPEVTCVVVDVSHEDPEVKFNWYVDGVEVHNAKTKPREEQYNSTY | NO: |
| | | RVVSVLTVLHQDWLNGKEYKCKVSNKALGAPIEKTISKAKGQPREPQVYTLP | 1142)×2-hu |
| | | PSRDELTKNQVSLTCLVKGFYPSDIAVEWESNGQPENNYKTTPPVLDSDGSF | IL2Ra(1- |
| | | FLYSKLTVDKSRWQQGNVFSCSVMHEALHNHYTQKSLSLSPGGGSGLRELHL | 219; M25I)- |
| | | DNN | GSGGGG(SEQ |
| | | | ID NO: |
| | | | 1138)-hu |
| | | | IgG1Fc- |
| | | | GGSGLREL |
| | | | HLDNN(SEQ |
| | | | ID NO: |
| | | | 1165) |
| 1044 | Construct | APTSSSTKKTQLQLEHLLLDLQMILNGINNYKNPKLTRMLTFKFYMPKKATE | Hu |
| | 0000 | LKHLQCLEEELKPLEEVLNLAQSKNFHLRPRDLISNINVIVLELKGSETTFM | IL2(C125S)- |
| | | CEYADETATIVEFLNRWITFSQSIISTLTSGGGGSGGGGGPLGVRGLRELHL | 2×(SG4) |
| | | DNNGELCDDDPPEIPHATFKAMAYKEGTILNCECKRGFRRIKSGSLYMLCTG | (SEQ ID |
| | | NSSHSSWDNQCQCTSSATRNTTKQVTPQPEEQKERKTTEMQSPMQPVDQASL | NO: 1143)- |
| | | PGHCREPPPWENEATERIYHFVVGQMVYYQCVQGYRALHRGPAESVCKMTHG | |
| | | KTRWTQPQLICTGEMETSQFPGEEKPQASPEGRPESETSCLVTTTDFQIQTE | MMPcs1- |
| | | MAATMETSIFTTEYQGSGGGGDKTHTCPPCPAPEAAGGPSVFLFPPKPKDTL | LRELHLDN |
| | | MISRTPEVTCVVVDVSHEDPEVKFNWYVDGVEVHNAKTKPREEQYNSTYRVV | N(SEQ ID |
| | | SVLTVLHQDWLNGKEYKCKVSNKALGAPIEKTISKAKGQPREPQVYTLPPSR | NO: 188)-hu |
| | | DELTKNQVSLTCLVKGFYPSDIAVEWESNGQPENNYKTTPPVLDSDGSFFLY | IL2Ra(1- |
| | | SKLTVDKSRWQQGNVFSCSVMHEALHNHYTQKSLSLSPG | 219; M25I)- |
| | | | GSGGGG(SEQ |
| | | | ID NO: |
| | | | 1138)-hu |
| | | | IgG1Fc |
| 1045 | Construct IIIII | APTSSSTKKTQLQLEHLLLDLQMILNGINNYKNPKLTRMLTFKFYMPKKATE | huIL2(C125 |
| | | LKHLQCLEEELKPLEEVLNLAQSKNFHLRPRDLISNINVIVLELKGSETTFM | S)- |
| | | CEYADETATIVEFLNRWITFSQSIISTLTSGGKLWVLPKGPLGVRGGGGGSG | SGGKLWV |
| | | GGGSELCDDDPPEIPHATFKAMAYKEGTILNCECKRGFRRIKSGSLYMLCTG | LPK(SEQ ID |
| | | NSSHSSWDNQCQCTSSATRNTTKQVTPQPEEQKERKTTEMQSPMQPVDQASL | NO: 1154)- |
| | | PGHCREPPPWENEATERIYHFVVGQMVYYQCVQGYRALHRGPAESVCKMTHG | MMPcs1- |
| | | KTRWTQPQLICTGEMETSQFPGEEKPQASPEGRPESETSCLVTTTDFQIQTE | 2×(G4S) |
| | | MAATMETSIFTTEYQGSGGGGDKTHTCPPCPAPEAAGGPSVFLFPPKPKDTL | (SEQ ID |
| | | MISRTPEVTCVVVDVSHEDPEVKFNWYVDGVEVHNAKTKPREEQYNSTYRVV | NO: 1142)- |
| | | SVLTVLHQDWLNGKEYKCKVSNKALGAPIEKTISKAKGQPREPQVYTLPPSR | hu IL2Ra(1- |
| | | DELTKNQVSLTCLVKGFYPSDIAVEWESNGQPENNYKTTPPVLDSDGSFFLY | 219; M25I)- |
| | | SKLTVDKSRWQQGNVFSCSVMHEALHNHYTQKSLSLSPGGGSGVRIQRKKEK | GSGGGG(SEQ |
| | | MKET | ID NO: |
| | | | 1138)- |
| | | | huIgG1- |
| | | | VRIQRKKE |
| | | | KMKET(SEQ |
| | | | ID NO: |
| | | | 1139) |
| 1046 | Construct | APTSSSTKKTQLQLEHLLLDLQMILNGINNYKNPKLTRMLTFKFYMPKKATE | huIL2(C125 |
| | JJJJJ | LKHLQCLEEELKPLEEVLNLAQSKNFHLRPRDLISNINVIVLELKGSETTFM | S)- |
| | | CEYADETATIVEFLNRWITFSQSIISTLTSGGKLWVLPKGPLGVRGGGGGSG | SGGKLWV |
| | | GGGSELCDDDPPEIPHATFKAMAYKEGTILNCECKRGFRRIKSGSLYMLCTG | LPK |
| | | NSSHSSWDNQCQCTSSATRNTTKQVTPQPEEQKERKTTEMQSPMQPVDQASL | (SEQ |
| | | PGHCREPPPWENEATERIYHFVVGQMVYYQCVQGYRALHRGPAESVCKMTHG | ID NO: |
| | | KTRWTQPQLICTGEMETSQFPGEEKPQASPEGRPESETSCLVTTTDFQIQTE | 1154)- |
| | | MAATMETSIFTTEYQGSGGGGDKTHTCPPCPAPEAAGGPSVFLFPPKPKDTL | MMPcs1- |
| | | MISRTPEVTCVVVDVSHEDPEVKFNWYVDGVEVHNAKTKPREEQYNSTYRVV | 2×(G4S)(SEQ |
| | | SVLTVLHQDWLNGKEYKCKVSNKALGAPIEKTISKAKGQPREPQVYTLPPSR | ID NO: |
| | | DELTKNQVSLTCLVKGFYPSDIAVEWESNGQPENNYKTTPPVLDSDGSFFLY | 1142)-hu |
| | | SKLTVDKSRWQQGNVFSCSVMHEALHNHYTQKSLSLSPGGGSGLRELHLDNN | IL2Ra(1- |
| | | | 219; M25I)- |
| | | | GSGGGG |
| | | | (SEQ ID |
| | | | NO: 1138)- |
| | | | huIgG1- |
| | | | LRELHLDN |
| | | | N (SEQ ID |
| | | | NO: 188) |
| 1047 | Construct | EPKSSDKTHTCPPCPAPEAAGGPSVFLFPPKPKDTLMISRTPEVTCVVVDVS | Fc knob- |
| | KKKKK | HEDPEVKFNWYVDGVEVHNAKTKPREEQYNSTYRVVSVLTVLHQDWLNGKEY | MMPcs1- |
| | | KCKVSNKALPAPIEKTISKAKGQPREPQVYTLPPSRDELTENQVSLTCLVKG | huIL2 |
| | | FYPSDIAVEWESNGQPENNYKTTPPVLDSDGSFFLYSWLTVDKSRWQQGNVF | |
| | | SCSVMHEALHNRFTQKSLSLSPGGGGSGGGGGPLGVRGGGGGSAPTSSSTKK | |
| | | TQLQLEHLLLDLQMILNGINNYKNPKLTRMLTFKFYMPKKATELKHLQCLEE | |
| | | ELKPLEEVLNLAQSKNFHLRPRDLISNINVIVLELKGSETTFMCEYADETAT | |
| | | IVEFLNRWITFSQSIISTLT | |
| 1048 | Construct | APTSSSTKKTQLQLEHLLLDLQMILNGINNYKNPKLTRMLTFKFYMPKKATE | h IL2 |
| | LLLLL | LKHLQCLEEELKPLEEVLNLAQSKNFHLRPRDLISNINVIVLELKGSETTFM | (C125S)- |
| | | CEYADETATIVEFLNRWITFSQSIISTLTVRIQRKKEKMKETGPLGVRGGGG | VRIQRKKE |
| | | GSGGGGSGGGGSELCDDDPPEIPHATFKAMAYKEGTILNCECKRGFRRIKSG | KMKET |
| | | SLYMLCTGNSSHSSWDNQCQCTSSATRNTTKQVTPQPEEQKERKTTEMQSPM | (SEQ ID |
| | | QPVDQASLPGHCREPPPWENEATERIYHFVVGQMVYYQCVQGYRALHRGPAE | NO: 1139)- |
| | | SVCKMTHGKTRWTQPQLICTGEMETSQFPGEEKPQASPEGRPESETSCLVTT | MMPcs1- |
| | | TDFQIQTEMAATMETSIFTTEYQGSGGGEPKSSDKTHTCPPCPAPELLGGPS | 3×(G4S)(SEQ |
| | | VFLFPPKPKDTLMISRTPEVTCVVVDVSHEDPEVKFNWYVDGVEVHNAKTKP | ID NO: |
| | | REEQYNSTYRVVSVLTVLHQDWLNGKEYKCKVSNKALPAPIEKTISKAKGQP | |
| | | REPQVYTLPPSRDELTENQVSLTCLVKGFYPSDIAVEWESNGQPENNYKTTP | 1142)- |
| | | PVLDSDGSFFLYSWLTVDKSRWQQGNVFSCSVMHEALHNRFTQKSLSLSPG | hIL2Ra(M25 |
| | | | I)-GSGGGG |
| | | | (SEQ ID |
| | | | NO: 1138)- |
| | | | hu IgG1 Fc |
| | | | knob |
| 1049 | Construct | APTSSSTKKTQLQLEHLLLDLQMILNGINNYKNPKLTRMLTFKFYMPKKATE | h IL2 |
| | MMMMM | LKHLQCLEEELKPLEEVLNLAQSKNFHLRPRDLISNINVIVLELKGSETTFM | (C125S)- |
| | | CEYADETATIVEFLNRWITFSQSIISTLTSGGKLWVLPKGPLGVRGGGGGSG | SGGKLWV |
| | | GGGSGGGGSELCDDDPPEIPHATFKAMAYKEGTILNCECKRGFRRIKSGSLY | LPK (SEQ |
| | | MLCTGNSSHSSWDNQCQCTSSATRNTTKQVTPQPEEQKERKTTEMQSPMQPV | ID NO: |
| | | DQASLPGHCREPPPWENEATERIYHFVVGQMVYYQCVQGYRALHRGPAESVC | 1154)- |
| | | KMTHGKTRWTQPQLICTGEMETSQFPGEEKPQASPEGRPESETSCLVTTTDF | MMPcs1- |
| | | QIQTEMAATMETSIFTTEYQGSGGGEPKSSDKTHTCPPCPAPELLGGPSVFL | 3×(G4S)(SEQ |
| | | FPPKPKDTLMISRTPEVTCVVVDVSHEDPEVKFNWYVDGVEVHNAKTKPREE | ID NO: |
| | | QYNSTYRVVSVLTVLHQDWLNGKEYKCKVSNKALPAPIEKTISKAKGQPREP | 1142)- |
| | | QVYTLPPSRDELTENQVSLTCLVKGFYPSDIAVEWESNGQPENNYKTTPPVL | hIL2Ra(M25 |
| | | DSDGSFFLYSWLTVDKSRWQQGNVFSCSVMHEALHNRFTQKSLSLSPG | I)-GSGGGG |
| | | | (SEQ ID |
| | | | NO: 1138)- |
| | | | hu IgG1 Fc |
| | | | knob |
| 1050 | Construct | APTSSSTKKTQLQLEHLLLDLQMILNGINNYKNPKLTRMLTFKFYMPKKATE | huil2- |
| | NNNNN | LKHLQCLEEELKPLEEVLNLAQSKNFHLRPRDLISNINVIVLELKGSETTFM | VRIQRKKE |
| | | CEYADETATIVEFLNRWITFSQSIISTLTVRIQRKKEKMKETGPLGVRGGGG | KMKET |
| | | GSGGGGSGGGGSAVNGTSQFTCFYNSRANISCVWSQDGALQDTSCQVHAWPD | (SEQ ID |
| | | RRRWNQTCELLPVSQASWACNLILGAPDSQKLTTVDIVTLRVLCREGVRWRV | NO: 1139)- |
| | | MAIQDFKPFENLRLMAPISLQVVHVETHRCNISWEISQASHYFERHLEFEAR | mmpcs1- |
| | | TLSPGHTWEEAPLLTLKQKQEWICLETLTPDTQYEFQVRVKPLQGEFTTWSP | il2Rb-Fc |
| | | WSQPLAFRTKPAALGKDGSGGGEPKSSDKTHTCPPCPAPELLGGPSVFLFPP | knob |
| | | KPKDTLMISRTPEVTCVVVDVSHEDPEVKFNWYVDGVEVHNAKTKPREEQYN | |
| | | STYRVVSVLTVLHQDWLNGKEYKCKVSNKALPAPIEKTISKAKGQPREPQVY | |
| | | TLPPSRDELTENQVSLTCLVKGFYPSDIAVEWESNGQPENNYKTTPPVLDSD | |
| | | GSFFLYSWLTVDKSRWQQGNVFSCSVMHEALHNRFTQKSLSLSPG | |
| 1051 | Construct | APTSSSTKKTQLQLEHLLLDLQMILNGINNYKNPKLTRMLTFKFYMPKKATE | huil2- |
| | 00000 | LKHLQCLEEELKPLEEVLNLAQSKNFHLRPRDLISNINVIVLELKGSETTFM | KLWVLPK |
| | | CEYADETATIVEFLNRWITFSQSIISTLTSGGKLWVLPKGPLGVRGGGGGSG | (SEQ ID |
| | | GGGSGGGGSAVNGTSQFTCFYNSRANISCVWSQDGALQDTSCQVHAWPDRRR | NO: 200)- |
| | | WNQTCELLPVSQASWACNLILGAPDSQKLTTVDIVTLRVLCREGVRWRVMAI | mmpcs1- |
| | | QDFKPFENLRLMAPISLQVVHVETHRCNISWEISQASHYFERHLEFEARTLS | il2Rb-Fc |
| | | PGHTWEEAPLLTLKQKQEWICLETLTPDTQYEFQVRVKPLQGEFTTWSPWSQ | knob |
| | | PLAFRTKPAALGKDGSGGGEPKSSDKTHTCPPCPAPELLGGPSVFLFPPKPK | |
| | | DTLMISRTPEVTCVVVDVSHEDPEVKFNWYVDGVEVHNAKTKPREEQYNSTY | |
| | | RVVSVLTVLHQDWLNGKEYKCKVSNKALPAPIEKTISKAKGQPREPQVYTLP | |
| | | PSRDELTENQVSLTCLVKGFYPSDIAVEWESNGQPENNYKTTPPVLDSDGSF | |
| | | FLYSWLTVDKSRWQQGNVFSCSVMHEALHNRFTQKSLSLSPG | |
| 1052 | Construct | EPKSSDKTHTCPPCPAPELLGGPSVFLFPPKPKDTLMISRTPEVTCVVVDVS | empty Fc |
| | PPPPP | HEDPEVKFNWYVDGVEVHNAKTKPREEQYNSTYRVVSVLTVLHQDWLNGKEY | hole |
| | | KCKVSNKALPAPIEKTISKAKGQPREPRVYTLPPSRDELTKNQVSLTCLVKG | |
| | | FYPSDIAVEWESNGQPENNYKTTPPVLVSDGSFTLYSKLTVDKSRWQQGNVF | |
| | | SCSVMHEALHNHYTQKSLSLSPG | |
| 1053 | Construct | AVNGTSQFTCFYNSRANISCVWSQDGALQDTSCQVHAWPDRRRWNQTCELLP | huIL2Rb |
| | QQQQQ | VSQASWACNLILGAPDSQKLTTVDIVTLRVLCREGVRWRVMAIQDFKPFENL | ECD- |
| | | RLMAPISLQVVHVETHRCNISWEISQASHYFERHLEFEARTLSPGHTWEEAP | 3×(G4S)(SEQ |
| | | LLTLKQKQEWICLETLTPDTQYEFQVRVKPLQGEFTTWSPWSQPLAFRTKPA | ID NO: |
| | | ALGKDGGGGSGGGGSGGGGSEPKSSDKTHTCPPCPAPELLGGPSVFLFPPKP | 1142)-Fc |
| | | KDTLMISRTPEVTCVVVDVSHEDPEVKFNWYVDGVEVHNAKTKPREEQYNST | Hole |
| | | YRVVSVLTVLHQDWLNGKEYKCKVSNKALPAPIEKTISKAKGQPREPRVYTL | |
| | | PPSRDELTKNQVSLTCLVKGFYPSDIAVEWESNGQPENNYKTTPPVLVSDGS | |
| | | FTLYSKLTVDKSRWQQGNVFSCSVMHEALHNHYTQKSLSLSPG | |
| 1054 | Construct | EPKSSDKTHTCPPCPAPELLGGPSVFLFPPKPKDTLMISRTPEVTCVVVDVS | Fc hole- |
| | TTTTT | HEDPEVKFNWYVDGVEVHNAKTKPREEQYNSTYRVVSVLTVLHQDWLNGKEY | B2G1 |
| | | KCKVSNKALPAPIEKTISKAKGQPREPRVYTLPPSRDELTKNQVSLTCLVKG | |
| | | FYPSDIAVEWESNGQPENNYKTTPPVLVSDGSFTLYSKLTVDKSRWQQGNVF | |
| | | SCSVMHEALHNHYTQKSLSLSPGGGGGSGGGGSGGGGSGGGGSGGGGSQVQL | |
| | | VESGGGLVQAGGSLRLSCAASGRTFSNYFAGWFRQPPGEEREFVASISWGGD | |
| | | RMFYTDAVKGRFTISRDNAKNTVDLQMNSLKPEDTAVYYCSADRFATALYNG | |
| | | NGNYWGQGTQVTVSS | |
| 1055 | Construct | EPKSSDKTHTCPPCPAPELLGGPSVFLFPPKPKDTLMISRTPEVTCVVVDVS | Fc hole- |
| | RRRRR | HEDPEVKFNWYVDGVEVHNAKTKPREEQYNSTYRVVSVLTVLHQDWLNGKEY | B2d2b |
| | | KCKVSNKALPAPIEKTISKAKGQPREPRVYTLPPSRDELTKNQVSLTCLVKG | |
| | | FYPSDIAVEWESNGQPENNYKTTPPVLVSDGSFTLYSKLTVDKSRWQQGNVF | |
| | | SCSVMHEALHNHYTQKSLSLSPGGGGGSGGGGSGGGGSGGGGSGGGGSEVQL | |
| | | VESGGGLVQAGGSLRLSCAASGRTFIGYTMGWFRQAPGKEREFVASIIWRGD | |
| | | RTRYADSVKGRFTISGDNAKNTVYLRMNSMKPEDTAVYYCAARSGSHFPSFD | |
| | | YWGQGTQVSVSS | |
| 1056 | Construct | EPKSSDKTHTCPPCPAPELLGGPSVFLFPPKPKDTLMISRTPEVTCVVVDVS | Fc hole- |
| | SSSSS | HEDPEVKFNWYVDGVEVHNAKTKPREEQYNSTYRVVSVLTVLHQDWLNGKEY | B2C10 |
| | | KCKVSNKALPAPIEKTISKAKGQPREPRVYTLPPSRDELTKNQVSLTCLVKG | |
| | | FYPSDIAVEWESNGQPENNYKTTPPVLVSDGSFTLYSKLTVDKSRWQQGNVF | |
| | | SCSVMHEALHNHYTQKSLSLSPGGGGGSGGGGSGGGGSGGGGSGGGGSEVQL | |
| | | VESGGGLVQPGGSLRLSCEVSGRILYIMGWFRQVPGKDREFVAGILWSSTKY | |
| | | GDFVNGRFTISRDNVKNTVSLQMNSLKPEDTAVYYCAAAIRRGQDIPTISSE | |
| | | YNYWGQGTQVTVSS | |
| 1057 | Construct | EPKSSDKTHTCPPCPAPELLGGPSVFLFPPKPKDTLMISRTPEVTCVVVDVS | Fc hole- |
| | UUUUU | HEDPEVKFNWYVDGVEVHNAKTKPREEQYNSTYRVVSVLTVLHQDWLNGKEY | B2C10- |
| | | KCKVSNKALPAPIEKTISKAKGQPREPRVYTLPPSRDELTKNQVSLTCLVKG | 5×(G4S)(SEQ |
| | | FYPSDIAVEWESNGQPENNYKTTPPVLVSDGSFTLYSKLTVDKSRWQQGNVF | ID NO: |
| | | SCSVMHEALHNHYTQKSLSLSPGGGGGSGGGGSGGGGSGGGGSGGGGSEVQL | 1142)- |
| | | VESGGGLVQPGGSLRLSCEVSGRILYIMGWFRQVPGKDREFVAGILWSSTKY | B2D2b |
| | | GDFVNGRFTISRDNVKNTVSLQMNSLKPEDTAVYYCAAAIRRGQDIPTISSE | |
| | | YNYWGQGTQVTVSSGGGGSGGGGSGGGGSGGGGSGGGGSEVQLVESGGGLVQ | |
| | | AGGSLRLSCAASGRTFIGYTMGWFRQAPGKEREFVASIIWRGDRTRYADSVK | |
| | | GRFTISGDNAKNTVYLRMNSMKPEDTAVYYCAARSGSHFPSFDYWGQGTQVS | |
| | | VSS | |
| 1058 | Construct | EPKSSDKTHTCPPCPAPELLGGPSVFLFPPKPKDTLMISRTPEVTCVVVDVS | Fc hole- |
| | VVVVV | HEDPEVKFNWYVDGVEVHNAKTKPREEQYNSTYRVVSVLTVLHQDWLNGKEY | B2G1- |
| | | KCKVSNKALPAPIEKTISKAKGQPREPRVYTLPPSRDELTKNQVSLTCLVKG | 5×(G4S)(SEQ |
| | | FYPSDIAVEWESNGQPENNYKTTPPVLVSDGSFTLYSKLTVDKSRWQQGNVF | ID NO: |
| | | SCSVMHEALHNHYTQKSLSLSPGGGGGSGGGGSGGGGSGGGGSGGGGSQVQL | 1142)- |
| | | VESGGGLVQAGGSLRLSCAASGRTFSNYFAGWFRQPPGEEREFVASISWGGD | B2D2b |
| | | RMFYTDAVKGRFTISRDNAKNTVDLQMNSLKPEDTAVYYCSADRFATALYNG | |
| | | NGNYWGQGTQVTVSSGGGGSGGGGSGGGGSGGGGSGGGGSEVQLVESGGGLV | |
| | | QAGGSLRLSCAASGRTFIGYTMGWFRQAPGKEREFVASIIWRGDRTRYADSV | |
| | | KGRFTISGDNAKNTVYLRMNSMKPEDTAVYYCAARSGSHFPSFDYWGQGTQV | |
| | | SVSS | |
| 1059 | Construct | EPKSSDKTHTCPPCPAPELLGGPSVFLFPPKPKDTLMISRTPEVTCVVVDVS | Fc hole- |
| | WWWWW | HEDPEVKFNWYVDGVEVHNAKTKPREEQYNSTYRVVSVLTVLHQDWLNGKEY | B2D2b- |
| | | KCKVSNKALPAPIEKTISKAKGQPREPRVYTLPPSRDELTKNQVSLTCLVKG | 5×(G4S)(SEQ |
| | | FYPSDIAVEWESNGQPENNYKTTPPVLVSDGSFTLYSKLTVDKSRWQQGNVF | ID NO: |
| | | SCSVMHEALHNHYTQKSLSLSPGGGGGSGGGGSGGGGSGGGGSGGGGSEVQL | 1142)-B2G1 |
| | | VESGGGLVQAGGSLRLSCAASGRTFIGYTMGWFRQAPGKEREFVASIIWRGD | |
| | | RTRYADSVKGRFTISGDNAKNTVYLRMNSMKPEDTAVYYCAARSGSHFPSFD | |
| | | YWGQGTQVSVSSGGGGGGGGSGGGGSGGGGSGGGGSQVQLVESGGGLVQAG | |
| | | GSLRLSCAASGRTFSNYFAGWFRQPPGEEREFVASISWGGDRMFYTDAVKGR | |
| | | FTISRDNAKNTVDLQMNSLKPEDTAVYYCSADRFATALYNGNGNYWGQGTQV | |
| | | TVSS | |
| 1060 | Construct | EPKSSDKTHTCPPCPAPELLGGPSVFLFPPKPKDTLMISRTPEVTCVVVDVS | Fc hole- |
| | XXXXX | HEDPEVKFNWYVDGVEVHNAKTKPREEQYNSTYRVVSVLTVLHQDWLNGKEY | B2D2b- |
| | | KCKVSNKALPAPIEKTISKAKGQPREPRVYTLPPSRDELTKNQVSLTCLVKG | 5×(G4S)(SEQ |
| | | FYPSDIAVEWESNGQPENNYKTTPPVLVSDGSFTLYSKLTVDKSRWQQGNVF | ID NO: |
| | | SCSVMHEALHNHYTQKSLSLSPGGGGGSGGGGSGGGGSGGGGSGGGGSEVQL | 1142)- |
| | | VESGGGLVQAGGSLRLSCAASGRTFIGYTMGWFRQAPGKEREFVASIIWRGD | B2C10 |
| | | RTRYADSVKGRFTISGDNAKNTVYLRMNSMKPEDTAVYYCAARSGSHFPSFD | |
| | | YWGQGTQVSVSSGGGGSGGGGSGGGGSGGGGSGGGGSEVQLVESGGGLVQPG | |
| | | GSLRLSCEVSGRILYIMGWFRQVPGKDREFVAGILWSSTKYGDFVNGRFTIS | |
| | | RDNVKNTVSLQMNSLKPEDTAVYYCAAAIRRGQDIPTISSEYNYWGQGTQVT | |
| | | VSS | |
| 1061 | Construct | EVQLVESGGGLVQPGGSLRLSCEVSGRILYIMGWFRQVPGKDREFVAGILWS | B2C10- |
| | YYYYY | STKYGDFVNGRFTISRDNVKNTVSLQMNSLKPEDTAVYYCAAAIRRGQDIPT | 5×(G4S)(SEQ |
| | | ISSEYNYWGQGTQVTVSSGGGGSGGGGSGGGGSGGGGSGGGGSEPKSSDKTH | ID NO: |
| | | TCPPCPAPELLGGPSVFLFPPKPKDTLMISRTPEVTCVVVDVSHEDPEVKFN | 1142)-Fc |
| | | WYVDGVEVHNAKTKPREEQYNSTYRVVSVLTVLHQDWLNGKEYKCKVSNKAL | Hole |
| | | PAPIEKTISKAKGQPREPRVYTLPPSRDELTKNQVSLTCLVKGFYPSDIAVE | |
| | | WESNGQPENNYKTTPPVLVSDGSFTLYSKLTVDKSRWQQGNVFSCSVMHEAL | |
| | | HNHYTQKSLSLSPG | |
| 1062 | Construct | QVQLVESGGGLVQAGGSLRLSCAASGRTFSNYFAGWFRQPPGEEREFVASIS | B2G1- |
| | ZZZZZ | WGGDRMFYTDAVKGRFTISRDNAKNTVDLQMNSLKPEDTAVYYCSADRFATA | 5×(G4S)(SEQ |
| | | LYNGNGNYWGQGTQVTVSSGGGGSGGGGSGGGGSGGGGSGGGGSEPKSSDKT | ID NO: |
| | | HTCPPCPAPELLGGPSVFLFPPKPKDTLMISRTPEVTCVVVDVSHEDPEVKF | |
| | | NWYVDGVEVHNAKTKPREEQYNSTYRVVSVLTVLHQDWLNGKEYKCKVSNKA | |
| | | LPAPIEKTISKAKGQPREPRVYTLPPSRDELTKNQVSLTCLVKGFYPSDIAV | 1142)-Fc |
| | | EWESNGQPENNYKTTPPVLVSDGSFTLYSKLTVDKSRWQQGNVFSCSVMHEA | Hole |
| | | LHNHYTQKSLSLSPG | |
| 1063 | Construct | EPKSSDKTHTCPPCPAPELLGGPSVFLFPPKPKDTLMISRTPEVTCVVVDVS | B2D2b- |
| | AAAAAA | HEDPEVKFNWYVDGVEVHNAKTKPREEQYNSTYRVVSVLTVLHQDWLNGKEY | 5×(G4S)(SEQ |
| | | KCKVSNKALPAPIEKTISKAKGQPREPRVYTLPPSRDELTKNQVSLTCLVKG | ID NO: |
| | | FYPSDIAVEWESNGQPENNYKTTPPVLVSDGSFTLYSKLTVDKSRWQQGNVF | 1142)-Fc |
| | | SCSVMHEALHNHYTQKSLSLSPGGGGGSGGGGSGGGGSGGGGSGGGGSEVQL | Hole |
| | | VESGGGLVQAGGSLRLSCAASGRTFIGYTMGWFRQAPGKEREFVASIIWRGD | |
| | | RTRYADSVKGRFTISGDNAKNTVYLRMNSMKPEDTAVYYCAARSGSHFPSFD | |
| | | YWGQGTQVSVSS | |
| 1064 | Construct | EPKSSDKTHTCPPCPAPELLGGPSVFLFPPKPKDTLMISRTPEVTCVVVDVS | Fc knob- |
| | BBBBBB | HEDPEVKFNWYVDGVEVHNAKTKPREEQYNSTYRVVSVLTVLHQDWLNGKEY | MMPcs1- |
| | | KCKVSNKALPAPIEKTISKAKGQPREPQVYTLPPSRDELTENQVSLTCLVKG | huIL2 |
| | | FYPSDIAVEWESNGQPENNYKTTPPVLDSDGSFFLYSWLTVDKSRWQQGNVF | (C125S) |
| | | SCSVMHEALHNRFTQKSLSLSPGGGGSGGGGGPLGVRGGGGGSAPTSSSTKK | |
| | | TQLQLEHLLLDLQMILNGINNYKNPKLTRMLTFKFYMPKKATELKHLQCLEE | |
| | | ELKPLEEVLNLAQSKNFHLRPRDLISNINVIVLELKGSETTFMCEYADETAT | |
| | | IVEFLNRWITFSQSIISTLT | |
| 1065 | Construct | APTSSSTKKTQLQLEHLLLDLQMILNGINNYKNPKLTRMLTFKFYMPKKATE | h IL2 |
| | CCCCCC | LKHLQCLEEELKPLEEVLNLAQSKNFHLRPRDLISNINVIVLELKGSETTFM | (C125S)- |
| | | CEYADETATIVEFLNRWITFSQSIISTLTSGGGGSGGGGGPLGVRGLRELHL | 2×(SG4)(SEQ |
| | | DNNGGGGSELCDDDPPEIPHATFKAMAYKEGTILNCECKRGFRRIKSGSLYM | ID NO: |
| | | LCTGNSSHSSWDNQCQCTSSATRNTTKQVTPQPEEQKERKTTEMQSPMQPVD | 1143)- |
| | | QASLPGHCREPPPWENEATERIYHFVVGQMVYYQCVQGYRALHRGPAESVCK | MMPcs1- |
| | | MTHGKTRWTQPQLICTGEMETSQFPGEEKPQASPEGRPESETSCLVTTTDFQ | LRELHLDN |
| | | IQTEMAATMETSIFTTEYQGSGGGEPKSSDKTHTCPPCPAPELLGGPSVFLF | N (SEQ ID |
| | | PPKPKDTLMISRTPEVTCVVVDVSHEDPEVKFNWYVDGVEVHNAKTKPREEQ | NO: 188)- |
| | | YNSTYRVVSVLTVLHQDWLNGKEYKCKVSNKALPAPIEKTISKAKGQPREPQ | 1×(G4S)(SEQ |
| | | VYTLPPSRDELTENQVSLTCLVKGFYPSDIAVEWESNGQPENNYKTTPPVLD | ID NO: |
| | | SDGSFFLYSWLTVDKSRWQQGNVFSCSVMHEALHNRFTQKSLSLSPG | 1142)- |
| | | | hIL2Ra(M25 |
| | | | I)-GSGGGG |
| | | | (SEQ ID |
| | | | NO: 1138)- |
| | | | hu IgG1 Fc |
| | | | knob |
| 1066 | Construct | APTSSSTKKTQLQLEHLLLDLQMILNGINNYKNPKLTRMLTFKFYMPKKATE | h IL2 |
| | DDDDDD | LKHLQCLEEELKPLEEVLNLAQSKNFHLRPRDLISNINVIVLELKGSETTFM | (C125S)- |
| | | CEYADETATIVEFLNRWITFSQSIISTLTVRIQRKKEKMKETGPLGVRGGGG | VRIQRKKE |
| | | GSGGGGSGGGGSELCDDDPPEIPHATFKAMAYKEGTILNCECKRGFRRIKSG | KMKET- |
| | | SLYMLCTGNSSHSSWDNQCQCTSSATRNTTKQVTPQPEEQKERKTTEMQSPM | MMPcs1- |
| | | QPVDQASLPGHCREPPPWENEATERIYHFVVGQMVYYQCVQGYRALHRGPAE | 3×(G4S)(SEQ |
| | | SVCKMTHGKTRWTQPQLICTGEMETSQFPGEEKPQASPEGRPESETSCLVTT | ID NO: |
| | | TDFQIQTEMAATMETSIFTTEYQGSGGGEPKSSDKTHTCPPCPAPELLGGPS | 1142)- |
| | | VFLFPPKPKDTLMISRTPEVTCVVVDVSHEDPEVKFNWYVDGVEVHNAKTKP | hIL2Ra(M25 |
| | | REEQYNSTYRVVSVLTVLHQDWLNGKEYKCKVSNKALPAPIEKTISKAKGQP | I)-GSGGGG |
| | | REPQVYTLPPSRDELTKNQVSLTCLVKGFYPSDIAVEWESNGQPENNYKTTP | (SEQ ID |
| | | PVLDSDGSFFLYSKLTVDKSRWQQGNVFSCSVMHEALHNHYTQKSLSLSPG | NO: 1138)- |
| | | | hu IgG1 Fc |
| 1067 | Construct | APTSSSTKKTQLQLEHLLLDLQMILNGINNYKNPKLTRMLTFKFYMPKKATE | h IL2 |
| | EEEEEE | LKHLQCLEEELKPLEEVLNLAQSKNFHLRPRDLISNINVIVLELKGSETTFM | (C125S)- |
| | | CEYADETATIVEFLNRWITFSQSIISTLTSGGKLWVLPKGPLGVRGGGGGSG | SGGKLWV |
| | | GGGSGGGGSELCDDDPPEIPHATFKAMAYKEGTILNCECKRGFRRIKSGSLY | LPK (SEQ |
| | | MLCTGNSSHSSWDNQCQCTSSATRNTTKQVTPQPEEQKERKTTEMQSPMQPV | ID NO: |
| | | DQASLPGHCREPPPWENEATERIYHFVVGQMVYYQCVQGYRALHRGPAESVC | 1154)- |
| | | KMTHGKTRWTQPQLICTGEMETSQFPGEEKPQASPEGRPESETSCLVTTTDF | MMPcs1- |
| | | QIQTEMAATMETSIFTTEYQGSGGGEPKSSDKTHTCPPCPAPELLGGPSVFL | 3×(G4S)(SEQ |
| | | FPPKPKDTLMISRTPEVTCVVVDVSHEDPEVKFNWYVDGVEVHNAKTKPREE | ID NO: |
| | | QYNSTYRVVSVLTVLHQDWLNGKEYKCKVSNKALPAPIEKTISKAKGQPREP | 1142)- |
| | | QVYTLPPSRDELTKNQVSLTCLVKGFYPSDIAVEWESNGQPENNYKTTPPVL | hIL2Ra(M25 |
| | | DSDGSFFLYSKLTVDKSRWQQGNVFSCSVMHEALHNHYTQKSLSLSPG | I)-GSGGGG |
| | | | (SEQ ID |
| | | | NO: 1138)- |
| | | | hu IgG1 Fc |
| 1068 | Construct | APTSSSTKKTQLQLEHLLLDLQMILNGINNYKNPKLTRMLTFKFYMPKKATE | h IL2 |
| | FFFFFF | LKHLQCLEEELKPLEEVLNLAQSKNFHLRPRDLISNINVIVLELKGSETTFM | (C125S)- |
| | | CEYADETATIVEFLNRWITFSQSIISTLTSGGGGSGGGGGPLGVRGLRELHL | 2×(SG4)(SEQ |
| | | DNNGGGGSELCDDDPPEIPHATFKAMAYKEGTILNCECKRGFRRIKSGSLYM | ID NO: |
| | | LCTGNSSHSSWDNQCQCTSSATRNTTKQVTPQPEEQKERKTTEMQSPMQPVD | 1143)- |
| | | QASLPGHCREPPPWENEATERIYHFVVGQMVYYQCVQGYRALHRGPAESVCK | MMPcs1- |
| | | MTHGKTRWTQPQLICTGEMETSQFPGEEKPQASPEGRPESETSCLVTTTDFQ | LRELHLDN |
| | | IQTEMAATMETSIFTTEYQGSGGGEPKSSDKTHTCPPCPAPELLGGPSVFLF | N (SEQ ID |
| | | PPKPKDTLMISRTPEVTCVVVDVSHEDPEVKFNWYVDGVEVHNAKTKPREEQ | NO: 188)- |
| | | YNSTYRVVSVLTVLHQDWLNGKEYKCKVSNKALPAPIEKTISKAKGQPREPQ | 1×(G4S)(SEQ |
| | | VYTLPPSRDELTKNQVSLTCLVKGFYPSDIAVEWESNGQPENNYKTTPPVLD | ID NO: |
| | | SDGSFFLYSKLTVDKSRWQQGNVFSCSVMHEALHNHYTQKSLSLSPG | 1142)- |
| | | | hIL2Ra(M25 |
| | | | I)-GSGGGG |
| | | | (SEQ ID |
| | | | NO: 1138)- |
| | | | hu IgG1 Fc |
| 1069 | Construct | EPKSSDKTHTCPPCPAPELLGGPSVFLFPPKPKDTLMISRTPEVTCVVVDVS | Fc hole- |
| | GGGGGG | HEDPEVKFNWYVDGVEVHNAKTKPREEQYNSTYRVVSVLTVLHQDWLNGKEY | 5×(G4S)(SEQ |
| | | KCKVSNKALPAPIEKTISKAKGQPREPRVYTLPPSRDELTKNQVSLTCLVKG | ID NO: |
| | | FYPSDIAVEWESNGQPENNYKTTPPVLVSDGSFTLYSKLTVDKSRWQQGNVF | 1142)-IL2Ra |
| | | SCSVMHEALHNHYTQKSLSLSPGGGGGSGGGGSGGGGSGGGGSGGGGSELCD | (1-219; |
| | | DDPPEIPHATFKAMAYKEGTILNCECKRGFRRIKSGSLYMLCTGNSSHSSWD | M25I) |
| | | NQCQCTSSATRNTTKQVTPQPEEQKERKTTEMQSPMQPVDQASLPGHCREPP | |
| | | PWENEATERIYHFVVGQMVYYQCVQGYRALHRGPAESVCKMTHGKTRWTQPQ | |
| | | LICTGEMETSQFPGEEKPQASPEGRPESETSCLVTTTDFQIQTEMAATMETS | |
| | | IFTTEYQ | |
| 1070 | Construct | EPKSSDKTHTCPPCPAPELLGGPSVFLFPPKPKDTLMISRTPEVTCVVVDVS | Fc hole- |
| | HHHHHH | HEDPEVKFNWYVDGVEVHNAKTKPREEQYNSTYRVVSVLTVLHQDWLNGKEY | 3×(G4S)(SEQ |
| | | KCKVSNKALPAPIEKTISKAKGQPREPRVYTLPPSRDELTKNQVSLTCLVKG | ID NO: |
| | | FYPSDIAVEWESNGQPENNYKTTPPVLVSDGSFTLYSKLTVDKSRWQQGNVF | 1142)-IL2Ra |
| | | SCSVMHEALHNHYTQKSLSLSPGGGGGSGGGGSGGGGSELCDDDPPEIPHAT | (1-219; |
| | | FKAMAYKEGTILNCECKRGFRRIKSGSLYMLCTGNSSHSSWDNQCQCTSSAT | M25I) |
| | | RNTTKQVTPQPEEQKERKTTEMQSPMQPVDQASLPGHCREPPPWENEATERI | |
| | | YHFVVGQMVYYQCVQGYRALHRGPAESVCKMTHGKTRWTQPQLICTGEMETS | |
| | | QFPGEEKPQASPEGRPESETSCLVTTTDFQIQTEMAATMETSIFTTEYQ | |
| 1071 | Construct | EPKSSDKTHTCPPCPAPELLGGPSVFLFPPKPKDTLMISRTPEVTCVVVDVS | Fc hole- |
| | IIIIII | HEDPEVKFNWYVDGVEVHNAKTKPREEQYNSTYRVVSVLTVLHQDWLNGKEY | B2C10- |
| | | KCKVSNKALPAPIEKTISKAKGQPREPRVYTLPPSRDELTKNQVSLTCLVKG | 5×(G4S)(SEQ |
| | | FYPSDIAVEWESNGQPENNYKTTPPVLVSDGSFTLYSKLTVDKSRWQQGNVF | ID NO: |
| | | SCSVMHEALHNHYTQKSLSLSPGGGGGSGGGGSGGGGSGGGGSGGGGSEVQL | 1142)-IL2Ra |
| | | VESGGGLVQPGGSLRLSCEVSGRILYIMGWFRQVPGKDREFVAGILWSSTKY | (1-219; |
| | | GDFVNGRFTISRDNVKNTVSLQMNSLKPEDTAVYYCAAAIRRGQDIPTISSE | M25I) |
| | | YNYWGQGTQVTVSSGGGGGGGGSGGGGSGGGGSGGGGSELCDDDPPEIPHA | |
| | | TFKAMAYKEGTILNCECKRGFRRIKSGSLYMLCTGNSSHSSWDNQCQCTSSA | |
| | | TRNTTKQVTPQPEEQKERKTTEMQSPMQPVDQASLPGHCREPPPWENEATER | |
| | | IYHFVVGQMVYYQCVQGYRALHRGPAESVCKMTHGKTRWTQPQLICTGEMET | |
| | | SQFPGEEKPQASPEGRPESETSCLVTTTDFQIQTEMAATMETSIFTTEYQ | |
| 1072 | Construct | EPKSSDKTHTCPPCPAPELLGGPSVFLFPPKPKDTLMISRTPEVTCVVVDVS | Fc hole- |
| | JJJJJJ | HEDPEVKFNWYVDGVEVHNAKTKPREEQYNSTYRVVSVLTVLHQDWLNGKEY | B2G1- |
| | | KCKVSNKALPAPIEKTISKAKGQPREPRVYTLPPSRDELTKNQVSLTCLVKG | 5×(G4S)(SEQ |
| | | FYPSDIAVEWESNGQPENNYKTTPPVLVSDGSFTLYSKLTVDKSRWQQGNVF | ID NO: |
| | | SCSVMHEALHNHYTQKSLSLSPGGGGGSGGGGSGGGGSGGGGSGGGGSQVQL | 1142)-IL2Ra |
| | | VESGGGLVQAGGSLRLSCAASGRTFSNYFAGWFRQPPGEEREFVASISWGGD | (1-219; |
| | | RMFYTDAVKGRFTISRDNAKNTVDLQMNSLKPEDTAVYYCSADRFATALYNG | M25I) |
| | | NGNYWGQGTQVTVSSGGGGSGGGGSGGGGSGGGGSGGGGSELCDDDPPEIPH | |
| | | ATFKAMAYKEGTILNCECKRGFRRIKSGSLYMLCTGNSSHSSWDNQCQCTSS | |
| | | ATRNTTKQVTPQPEEQKERKTTEMQSPMQPVDQASLPGHCREPPPWENEATE | |
| | | RIYHFVVGQMVYYQCVQGYRALHRGPAESVCKMTHGKTRWTQPQLICTGEME | |
| | | TSQFPGEEKPQASPEGRPESETSCLVTTTDFQIQTEMAATMETSIFTTEYQ | |
| 1073 | Construct | EPKSSDKTHTCPPCPAPELLGGPSVFLFPPKPKDTLMISRTPEVTCVVVDVS | Fc hole- |
| | KKKKKK | HEDPEVKFNWYVDGVEVHNAKTKPREEQYNSTYRVVSVLTVLHQDWLNGKEY | B2D2b- |
| | | KCKVSNKALPAPIEKTISKAKGQPREPRVYTLPPSRDELTKNQVSLTCLVKG | 5×(G4S)(SEQ |
| | | FYPSDIAVEWESNGQPENNYKTTPPVLVSDGSFTLYSKLTVDKSRWQQGNVF | ID NO: |
| | | SCSVMHEALHNHYTQKSLSLSPGGGGGSGGGGSGGGGSGGGGSGGGGSEVQL | 1142)-IL2Ra |
| | | VESGGGLVQAGGSLRLSCAASGRTFIGYTMGWFRQAPGKEREFVASIIWRGD | (1-219; |
| | | RTRYADSVKGRFTISGDNAKNTVYLRMNSMKPEDTAVYYCAARSGSHFPSFD | M25I) |
| | | YWGQGTQVSVSSGGGGSGGGGSGGGGSGGGGSGGGGSELCDDDPPEIPHATF | |
| | | KAMAYKEGTILNCECKRGFRRIKSGSLYMLCTGNSSHSSWDNQCQCTSSATR | |
| | | NTTKQVTPQPEEQKERKTTEMQSPMQPVDQASLPGHCREPPPWENEATERIY | |
| | | HFVVGQMVYYQCVQGYRALHRGPAESVCKMTHGKTRWTQPQLICTGEMETSQ | |
| | | FPGEEKPQASPEGRPESETSCLVTTTDFQIQTEMAATMETSIFTTEYQ | |
| 1074 | Construct | EPKSSDKTHTCPPCPAPELLGGPSVFLFPPKPKDTLMISRTPEVTCVVVDVS | Fc hole- |
| | LLLLLL | HEDPEVKFNWYVDGVEVHNAKTKPREEQYNSTYRVVSVLTVLHQDWLNGKEY | B2C10- |
| | | KCKVSNKALPAPIEKTISKAKGQPREPRVYTLPPSRDELTKNQVSLTCLVKG | 5×(G4S)(SEQ |
| | | FYPSDIAVEWESNGQPENNYKTTPPVLVSDGSFTLYSKLTVDKSRWQQGNVF | ID NO: |
| | | SCSVMHEALHNHYTQKSLSLSPGGGGGSGGGGSGGGGSGGGGSGGGGSEVQL | 1142)-B1C3 |
| | | VESGGGLVQPGGSLRLSCEVSGRILYIMGWFRQVPGKDREFVAGILWSSTKY | |
| | | GDFVNGRFTISRDNVKNTVSLQMNSLKPEDTAVYYCAAAIRRGQDIPTISSE | |
| | | YNYWGQGTQVTVSSGGGGSGGGGSGGGGSGGGGSGGGGSEVQLVESGGGLVQ | |
| | | PGESLRLSCLASRTLSTFNVMAWYRQAPEKERELVAHVTNGTTLVADSVKGR | |
| | | FTISRDYTKNTVDLQMSKLKPEDTAVYYCRFWRGRYEYWGQGTQVTVSS | |
| 1075 | Construct | EPKSSDKTHTCPPCPAPELLGGPSVFLFPPKPKDTLMISRTPEVTCVVVDVS | Fc hole- |
| | MMMMMM | HEDPEVKFNWYVDGVEVHNAKTKPREEQYNSTYRVVSVLTVLHQDWLNGKEY | B2G1- |
| | | KCKVSNKALPAPIEKTISKAKGQPREPRVYTLPPSRDELTKNQVSLTCLVKG | 5×(G4S)(SEQ |
| | | FYPSDIAVEWESNGQPENNYKTTPPVLVSDGSFTLYSKLTVDKSRWQQGNVF | ID NO: |
| | | SCSVMHEALHNHYTQKSLSLSPGGGGGSGGGGSGGGGSGGGGSGGGGSQVQL | 1142)-B1C3 |
| | | VESGGGLVQAGGSLRLSCAASGRTFSNYFAGWFRQPPGEEREFVASISWGGD | |
| | | RMFYTDAVKGRFTISRDNAKNTVDLQMNSLKPEDTAVYYCSADRFATALYNG | |
| | | NGNYWGQGTQVTVSSGGGGSGGGGSGGGGSGGGGGGGGSEVQLVESGGGLV | |
| | | QPGESLRLSCLASRTLSTFNVMAWYRQAPEKERELVAHVTNGTTLVADSVKG | |
| | | RFTISRDYTKNTVDLQMSKLKPEDTAVYYCRFWRGRYEYWGQGTQVTVSS | |
| 1076 | Construct | EPKSSDKTHTCPPCPAPELLGGPSVFLFPPKPKDTLMISRTPEVTCVVVDVS | Fc hole- |
| | NNNNNN | HEDPEVKFNWYVDGVEVHNAKTKPREEQYNSTYRVVSVLTVLHQDWLNGKEY | B2D2b- |
| | | KCKVSNKALPAPIEKTISKAKGQPREPRVYTLPPSRDELTKNQVSLTCLVKG | 5×(G4S)(SEQ |
| | | FYPSDIAVEWESNGQPENNYKTTPPVLVSDGSFTLYSKLTVDKSRWQQGNVF | ID NO: |
| | | SCSVMHEALHNHYTQKSLSLSPGGGGGSGGGGSGGGGGGGGSGGGGSEVQL | 1142)-B1C3 |
| | | VESGGGLVQAGGSLRLSCAASGRTFIGYTMGWFRQAPGKEREFVASIIWRGD | |
| | | RTRYADSVKGRFTISGDNAKNTVYLRMNSMKPEDTAVYYCAARSGSHFPSFD | |
| | | YWGQGTQVSVSSGGGGSGGGGSGGGGSGGGGSGGGGSQVQLVESGGGLVQAG | |
| | | GSLRLSCAASGRTFSNYFAGWFRQPPGEEREFVASISWGGDRMFYTDAVKGR | |
| | | FTISRDNAKNTVDLQMNSLKPEDTAVYYCSADRFATALYNGNGNYWGQGTQV | |
| | | TVSS | |
| 1077 | Construct | ELCDDDPPEIPHATFKAMAYKEGTILNCECKRGFRRIKSGSLYMLCTGNSSH | huIL2Ra(M2 |
| | 000000 | SSWDNQCQCTSSATRNTTKQVTPQPEEQKERKTTEMQSPMQPVDQASLPGHC | 51; 1-219)- |
| | | REPPPWENEATERIYHFVVGQMVYYQCVQGYRALHRGPAESVCKMTHGKTRW | 3×(G4S)(SEQ |
| | | TQPQLICTGEMETSQFPGEEKPQASPEGRPESETSCLVTTTDFQIQTEMAAT | ID NO: |
| | | METSIFTTEYQGGGGSGGGGSGGGGSEPKSSDKTHTCPPCPAPELLGGPSVF | |
| | | LFPPKPKDTLMISRTPEVTCVVVDVSHEDPEVKFNWYVDGVEVHNAKTKPRE | |
| | | EQYNSTYRVVSVLTVLHQDWLNGKEYKCKVSNKALPAPIEKTISKAKGQPRE | 1142)-Fc |
| | | PRVYTLPPSRDELTKNQVSLTCLVKGFYPSDIAVEWESNGQPENNYKTTPPV | Hole |
| | | LVSDGSFTLYSKLTVDKSRWQQGNVFSCSVMHEALHNHYTQKSLSLSPG | |
| 1078 | Construct | ELCDDDPPEIPHATFKAMAYKEGTILNCECKRGFRRIKSGSLYMLCTGNSSH | huIL2Ra(M2 |
| | PPPPPP | SSWDNQCQCTSSATRNTTKQVTPQPEEQKERKTTEMQSPMQPVDQASLPGHC | 51; 1-219)- |
| | | REPPPWENEATERIYHFVVGQMVYYQCVQGYRALHRGPAESVCKMTHGKTRW | 5×(G4S)(SEQ |
| | | TQPQLICTGEMETSQFPGEEKPQASPEGRPESETSCLVTTTDFQIQTEMAAT | ID NO: |
| | | METSIFTTEYQGGGGSGGGGSGGGGSGGGGSGGGGSEPKSSDKTHTCPPCPA | 1142)-Fc |
| | | PELLGGPSVFLFPPKPKDTLMISRTPEVTCVVVDVSHEDPEVKFNWYVDGVE | Hole |
| | | VHNAKTKPREEQYNSTYRVVSVLTVLHQDWLNGKEYKCKVSNKALPAPIEKT | |
| | | ISKAKGQPREPRVYTLPPSRDELTKNQVSLTCLVKGFYPSDIAVEWESNGQP | |
| | | ENNYKTTPPVLVSDGSFTLYSKLTVDKSRWQQGNVFSCSVMHEALHNHYTQK | |
| | | SLSLSPG | |
| 1079 | Construct | APTSSSTKKTQLQLEHLLLDLQMILNGINNYKNPKLTRMLTFKFYMPKKATE | huIL2(C125 |
| | QQQQQQ | LKHLQCLEEELKPLEEVLNLAQSKNFHLRPRDLISNINVIVLELKGSETTFM | S)- |
| | | CEYADETATIVEFLNRWITFSQSIISTLTSGGGGSGGGGGPLGVRGGGGGSG | 3×(G4S)(SEQ |
| | | GGGSEPKSSDKTHTCPPCPAPELLGGPSVFLFPPKPKDTLMISRTPEVTCVV | ID NO: |
| | | VDVSHEDPEVKFNWYVDGVEVHNAKTKPREEQYNSTYRVVSVLTVLHQDWLN | 1142)-Fc |
| | | GKEYKCKVSNKALPAPIEKTISKAKGQPREPQVYTLPPSRDELTENQVSLTC | knob |
| | | LVKGFYPSDIAVEWESNGQPENNYKTTPPVLDSDGSFFLYSWLTVDKSRWQQ | |
| | | GNVFSCSVMHEALHNRFTQKSLSLSPG | |
| 1080 | Construct | EPKSSDKTHTCPPCPAPELLGGPSVFLFPPKPKDTLMISRTPEVTCVVVDVS | Fc knob- |
| | RRRRRR | HEDPEVKFNWYVDGVEVHNAKTKPREEQYNSTYRVVSVLTVLHQDWLNGKEY | VRIQRKKE |
| | | KCKVSNKALPAPIEKTISKAKGQPREPQVYTLPPSRDELTENQVSLTCLVKG | KMKET |
| | | FYPSDIAVEWESNGQPENNYKTTPPVLDSDGSFFLYSWLTVDKSRWQQGNVF | (SEQ ID |
| | | SCSVMHEALHNRFTQKSLSLSPGGGGSVRIQRKKEKMKETGGGGGPLGVRGG | NO: 1139)- |
| | | GGGSAPTSSSTKKTQLQLEHLLLDLQMILNGINNYKNPKLTRMLTFKFYMPK | MMPcs1- |
| | | KATELKHLQCLEEELKPLEEVLNLAQSKNFHLRPRDLISNINVIVLELKGSE | huIL2 |
| | | TTFMCEYADETATIVEFLNRWITFSQSIISTLT | (C125S) |
| 1081 | Construct | EPKSSDKTHTCPPCPAPELLGGPSVFLFPPKPKDTLMISRTPEVTCVVVDVS | Fc knob- |
| | SSSSSS | HEDPEVKFNWYVDGVEVHNAKTKPREEQYNSTYRVVSVLTVLHQDWLNGKEY | SGGKLWV |
| | | KCKVSNKALPAPIEKTISKAKGQPREPQVYTLPPSRDELTENQVSLTCLVKG | LPK (SEQ |
| | | FYPSDIAVEWESNGQPENNYKTTPPVLDSDGSFFLYSWLTVDKSRWQQGNVF | ID NO: |
| | | SCSVMHEALHNRFTQKSLSLSPGGGGSKLWVLPKGGGGGPLGVRGGGGGSAP | 1154)- |
| | | TSSSTKKTQLQLEHLLLDLQMILNGINNYKNPKLTRMLTFKFYMPKKATELK | MMPcs1- |
| | | HLQCLEEELKPLEEVLNLAQSKNFHLRPRDLISNINVIVLELKGSETTFMCE | huIL2 |
| | | YADETATIVEFLNRWITFSQSIISTLT | (C125S) |
| 1082 | Construct | EPKSSDKTHTCPPCPAPELLGGPSVFLFPPKPKDTLMISRTPEVTCVVVDVS | Fc knob- |
| | TTTTTT | HEDPEVKFNWYVDGVEVHNAKTKPREEQYNSTYRVVSVLTVLHQDWLNGKEY | LRELHLDN |
| | | KCKVSNKALPAPIEKTISKAKGQPREPQVYTLPPSRDELTENQVSLTCLVKG | N (SEQ ID |
| | | FYPSDIAVEWESNGQPENNYKTTPPVLDSDGSFFLYSWLTVDKSRWQQGNVF | NO: 188)- |
| | | SCSVMHEALHNRFTQKSLSLSPGGGGSLRELHLDNNGGGGGPLGVRGGGGGS | MMPcs1- |
| | | APTSSSTKKTQLQLEHLLLDLQMILNGINNYKNPKLTRMLTFKFYMPKKATE | huIL2 |
| | | LKHLQCLEEELKPLEEVLNLAQSKNFHLRPRDLISNINVIVLELKGSETTFM | (C125S) |
| | | CEYADETATIVEFLNRWITFSQSIISTLT | |
| 1083 | Construct | APTSSSTKKTQLQLEHLLLDLQMILNGINNYKNPKLTRMLTFKFYMPKKATE | huIL2 |
| | UUUUUU | LKHLQCLEEELKPLEEVLNLAQSKNFHLRPRDLISNINVIVLELKGSETTFM | (C125S)- |
| | | CEYADETATIVEFLNRWITFSQSIISTLTGGGGSGGGGGPLGVRGGGSLREL | 2×(G4S)(SEQ |
| | | HLDNNGGGGSEPKSSDKTHTCPPCPAPELLGGPSVFLFPPKPKDTLMISRTP | ID NO: |
| | | EVTCVVVDVSHEDPEVKFNWYVDGVEVHNAKTKPREEQYNSTYRVVSVLTVL | 1142)- |
| | | HQDWLNGKEYKCKVSNKALPAPIEKTISKAKGQPREPQVYTLPPSRDELTEN | MMPcs1- |
| | | QVSLTCLVKGFYPSDIAVEWESNGQPENNYKTTPPVLDSDGSFFLYSWLTVD | LRELHLDN |
| | | KSRWQQGNVFSCSVMHEALHNRFTQKSLSLSPG | N (SEQ ID |
| | | | NO: 188)- |
| | | | 1×(G4S)(SEQ |
| | | | ID NO: |
| | | | 1142)-Fc |
| | | | knob |
| 1084 | Construct | EPKSSDKTHTCPPCPAPELLGGPSVFLFPPKPKDTLMISRTPEVTCVVVDVS | Fc hole- |
| | VVVVVV | HEDPEVKFNWYVDGVEVHNAKTKPREEQYNSTYRVVSVLTVLHQDWLNGKEY | B2G1-GSGG |
| | | KCKVSNKALPAPIEKTISKAKGQPREPRVYTLPPSRDELTKNQVSLTCLVKG | (SEQ ID |
| | | FYPSDIAVEWESNGQPENNYKTTPPVLVSDGSFTLYSKLTVDKSRWQQGNVF | NO: 1166)- |
| | | SCSVMHEALHNHYTQKSLSLSPGGGGGSGGGGSGGGGSGGGGSGGGGSQVQL | VRIQRKKE |
| | | VESGGGLVQAGGSLRLSCAASGRTFSNYFAGWFRQPPGEEREFVASISWGGD | KMKET |
| | | RMFYTDAVKGRFTISRDNAKNTVDLQMNSLKPEDTAVYYCSADRFATALYNG | (SEQ ID |
| | | NGNYWGQGTQVTVSSGSGGVRIQRKKEKMKET | NO: 1139) |
| 1085 | Construct | EPKSSDKTHTCPPCPAPELLGGPSVFLFPPKPKDTLMISRTPEVTCVVVDVS | Fc hole- |
| | WWWWWW | HEDPEVKFNWYVDGVEVHNAKTKPREEQYNSTYRVVSVLTVLHQDWLNGKEY | B2G1-GSGG |
| | | KCKVSNKALPAPIEKTISKAKGQPREPRVYTLPPSRDELTKNQVSLTCLVKG | (SEQ ID |
| | | FYPSDIAVEWESNGQPENNYKTTPPVLVSDGSFTLYSKLTVDKSRWQQGNVF | NO: 1166)- |
| | | SCSVMHEALHNHYTQKSLSLSPGGGGGSGGGGSGGGGSGGGGSGGGGSQVQL | LRELHLDN |
| | | VESGGGLVQAGGSLRLSCAASGRTFSNYFAGWFRQPPGEEREFVASISWGGD | N (SEQ ID |
| | | RMFYTDAVKGRFTISRDNAKNTVDLQMNSLKPEDTAVYYCSADRFATALYNG | NO: 188) |
| | | NGNYWGQGTQVTVSSGSGGLRELHLDNN | |
| 1086 | Construct | EPKSSDKTHTCPPCPAPELLGGPSVFLFPPKPKDTLMISRTPEVTCVVVDVS | Fc hole- |
| | XXXXXX | HEDPEVKFNWYVDGVEVHNAKTKPREEQYNSTYRVVSVLTVLHQDWLNGKEY | B2G1-GSGG |
| | | KCKVSNKALPAPIEKTISKAKGQPREPRVYTLPPSRDELTKNQVSLTCLVKG | (SEQ ID |
| | | FYPSDIAVEWESNGQPENNYKTTPPVLVSDGSFTLYSKLTVDKSRWQQGNVF | NO: 1166)- |
| | | SCSVMHEALHNHYTQKSLSLSPGGGGGSGGGGSGGGGSGGGGSGGGGSQVQL | KLWVLPK |
| | | VESGGGLVQAGGSLRLSCAASGRTFSNYFAGWFRQPPGEEREFVASISWGGD | (SEQ ID |
| | | RMFYTDAVKGRFTISRDNAKNTVDLQMNSLKPEDTAVYYCSADRFATALYNG | NO: 200) |
| | | NGNYWGQGTQVTVSSGSGGKLWVLPK | |
| 1087 | Construct | EPKSSDKTHTCPPCPAPELLGGPSVFLFPPKPKDTLMISRTPEVTCVVVDVS | Fc hole- |
| | | HEDPEVKFNWYVDGVEVHNAKTKPREEQYNSTYRVVSVLTVLHQDWLNGKEY | B2C10- |
| | | KCKVSNKALPAPIEKTISKAKGQPREPRVYTLPPSRDELTKNQVSLTCLVKG | GSGG (SEQ |
| | | FYPSDIAVEWESNGQPENNYKTTPPVLVSDGSFTLYSKLTVDKSRWQQGNVF | ID NO: |
| | | SCSVMHEALHNHYTQKSLSLSPGGGGGSGGGGSGGGGSGGGGSGGGGSEVQL | 1166)- |
| | | VESGGGLVQPGGSLRLSCEVSGRILYIMGWFRQVPGKDREFVAGILWSSTKY | VRIQRKKE |
| | | GDFVNGRFTISRDNVKNTVSLQMNSLKPEDTAVYYCAAAIRRGQDIPTISSE | KMKET |
| | | YNYWGQGTQVTVSSGSGGVRIQRKKEKMKET | (SEQ ID |
| | | | NO: 1139) |
| 1088 | Construct | EPKSSDKTHTCPPCPAPELLGGPSVFLFPPKPKDTLMISRTPEVTCVVVDVS | Fc hole- |
| | ZZZZZZ | HEDPEVKFNWYVDGVEVHNAKTKPREEQYNSTYRVVSVLTVLHQDWLNGKEY | B2C10- |
| | | KCKVSNKALPAPIEKTISKAKGQPREPRVYTLPPSRDELTKNQVSLTCLVKG | GSGG (SEQ |
| | | FYPSDIAVEWESNGQPENNYKTTPPVLVSDGSFTLYSKLTVDKSRWQQGNVF | ID NO: |
| | | SCSVMHEALHNHYTQKSLSLSPGGGGGSGGGGSGGGGSGGGGSGGGGSEVQL | 1166)- |
| | | VESGGGLVQPGGSLRLSCEVSGRILYIMGWFRQVPGKDREFVAGILWSSTKY | LRELHLDN |
| | | GDFVNGRFTISRDNVKNTVSLQMNSLKPEDTAVYYCAAAIRRGQDIPTISSE | N (SEQ ID |
| | | YNYWGQGTQVTVSSGSGGLRELHLDNN | NO: 188) |
| 1089 | Construct | EPKSSDKTHTCPPCPAPELLGGPSVFLFPPKPKDTLMISRTPEVTCVVVDVS | Fc hole- |
| | AAAAAAA | HEDPEVKFNWYVDGVEVHNAKTKPREEQYNSTYRVVSVLTVLHQDWLNGKEY | B2C10- |
| | | KCKVSNKALPAPIEKTISKAKGQPREPRVYTLPPSRDELTKNQVSLTCLVKG | GSGG |
| | | FYPSDIAVEWESNGQPENNYKTTPPVLVSDGSFTLYSKLTVDKSRWQQGNVF | (SEQ |
| | | SCSVMHEALHNHYTQKSLSLSPGGGGGSGGGGSGGGGSGGGGSGGGGSEVQL | ID NO: |
| | | VESGGGLVQPGGSLRLSCEVSGRILYIMGWFRQVPGKDREFVAGILWSSTKY | 1166)- |
| | | GDFVNGRFTISRDNVKNTVSLQMNSLKPEDTAVYYCAAAIRRGQDIPTISSE | KLWVLPK |
| | | YNYWGQGTQVTVSSGSGGKLWVLPK | (SEQ ID |
| | | | NO: 200) |
| 1090 | Construct | EPKSSDKTHTCPPCPAPELLGGPSVFLFPPKPKDTLMISRTPEVTCVVVDVS | Fc hole- |
| | BBBBBBB | HEDPEVKFNWYVDGVEVHNAKTKPREEQYNSTYRVVSVLTVLHQDWLNGKEY | B2D2b- |
| | | KCKVSNKALPAPIEKTISKAKGQPREPRVYTLPPSRDELTKNQVSLTCLVKG | GSGG (SEQ |
| | | FYPSDIAVEWESNGQPENNYKTTPPVLVSDGSFTLYSKLTVDKSRWQQGNVF | ID NO: |
| | | SCSVMHEALHNHYTQKSLSLSPGGGGGSGGGGSGGGGSGGGGSGGGGSEVQL | 1166)- |
| | | VESGGGLVQAGGSLRLSCAASGRTFIGYTMGWFRQAPGKEREFVASIIWRGD | VRIQRKKE |
| | | RTRYADSVKGRFTISGDNAKNTVYLRMNSMKPEDTAVYYCAARSGSHFPSFD | KMKET |
| | | YWGQGTQVSVSSGSGGVRIQRKKEKMKET | (SEQ ID |
| | | | NO: 1139) |
| 1091 | Construct | SEPKSSDKTHTCPPCPAPELLGGPSVFLFPPKPKDTLMISRTPEVTCVVVDV | Fc hole- |
| | CCCCCCC | SHEDPEVKFNWYVDGVEVHNAKTKPREEQYNSTYRVVSVLTVLHQDWLNGKE | B2D2b- |
| | | YKCKVSNKALPAPIEKTISKAKGQPREPRVYTLPPSRDELTKNQVSLTCLVK | GSGG (SEQ |
| | | GFYPSDIAVEWESNGQPENNYKTTPPVLVSDGSFTLYSKLTVDKSRWQQGNV | ID NO: |
| | | FSCSVMHEALHNHYTQKSLSLSPGGGGGSGGGGSGGGGSGGGGSGGGGSEVQ | 1166)- |
| | | LVESGGGLVQAGGSLRLSCAASGRTFIGYTMGWFRQAPGKEREFVASIIWRG | LRELHLDN |
| | | DRTRYADSVKGRFTISGDNAKNTVYLRMNSMKPEDTAVYYCAARSGSHFPSF | N (SEQ ID |
| | | DYWGQGTQVSVSSGSGGLRELHLDNN | NO: 188) |
| 1092 | Construct | EPKSSDKTHTCPPCPAPELLGGPSVFLFPPKPKDTLMISRTPEVTCVVVDVS | Fc hole- |
| | DDDDDDD | HEDPEVKFNWYVDGVEVHNAKTKPREEQYNSTYRVVSVLTVLHQDWLNGKEY | B2D2b- |
| | | KCKVSNKALPAPIEKTISKAKGQPREPRVYTLPPSRDELTKNQVSLTCLVKG | GSGG (SEQ |
| | | FYPSDIAVEWESNGQPENNYKTTPPVLVSDGSFTLYSKLTVDKSRWQQGNVF | ID NO: |
| | | SCSVMHEALHNHYTQKSLSLSPGGGGGSGGGGSGGGGSGGGGSGGGGSEVQL | 1166)- |
| | | VESGGGLVQAGGSLRLSCAASGRTFIGYTMGWFRQAPGKEREFVASIIWRGD | KLWVLPK |
| | | RTRYADSVKGRFTISGDNAKNTVYLRMNSMKPEDTAVYYCAARSGSHFPSFD | (SEQ ID |
| | | YWGQGTQVSVSSGSGGKLWVLPK | NO: 200) |
| 1093 | Construct | MGWSCIILFLVATATGVHSEPKSSDKTHTCPPCPAPELLGGPSVFLFPPKPK | Fc hole- |
| | EEEEEEE | DTLMISRTPEVTCVVVDVSHEDPEVKFNWYVDGVEVHNAKTKPREEQYNSTY | B2D2b- |
| | | RVVSVLTVLHQDWLNGKEYKCKVSNKALPAPIEKTISKAKGQPREPRVYTLP | 2×(G4S)(SEQ |
| | | PSRDELTKNQVSLTCLVKGFYPSDIAVEWESNGQPENNYKTTPPVLVSDGSF | ID NO: |
| | | TLYSKLTVDKSRWQQGNVFSCSVMHEALHNHYTQKSLSLSPGGGGGSGGGGS | 1142)- |
| | | GGGGSGGGGSGGGGSEVQLVESGGGLVQAGGSLRLSCAASGRTFIGYTMGWF | MMPcs1- |
| | | RQAPGKEREFVASIIWRGDRTRYADSVKGRFTISGDNAKNTVYLRMNSMKPE | 3×(G4S)(SEQ |
| | | DTAVYYCAARSGSHFPSFDYWGQGTQVSVSSGGGGSGGGGSGPLGVRGGGGS | ID NO: |
| | | GGGGSGGGGSEVQLVESGGGLVQPGGSLRLSCEVSGRILYIMGWFRQVPGKD | 1142)- |
| | | REFVAGILWSSTKYGDFVNGRFTISRDNVKNTVSLQMNSLKPEDTAVYYCAA | B2C10 |
| | | AIRRGQDIPTISSEYNYWGQGTQVTVSS | |
| 1094 | Construct | MGWSCIILFLVATATGVHSEPKSSDKTHTCPPCPAPELLGGPSVFLFPPKPK | Fc hole- |
| | FFFFFFF | DTLMISRTPEVTCVVVDVSHEDPEVKFNWYVDGVEVHNAKTKPREEQYNSTY | B2C10- |
| | | RVVSVLTVLHQDWLNGKEYKCKVSNKALPAPIEKTISKAKGQPREPRVYTLP | 2×(G4S)(SEQ |
| | | PSRDELTKNQVSLTCLVKGFYPSDIAVEWESNGQPENNYKTTPPVLVSDGSF | ID NO: |
| | | TLYSKLTVDKSRWQQGNVFSCSVMHEALHNHYTQKSLSLSPGGGGGSGGGGS | 1142)- |
| | | GGGGGGGGSGGGGSEVQLVESGGGLVQPGGSLRLSCEVSGRILYIMGWFRQ | MMPcs1- |
| | | VPGKDREFVAGILWSSTKYGDFVNGRFTISRDNVKNTVSLQMNSLKPEDTAV | 3×(G4S)(SEQ |
| | | YYCAAAIRRGQDIPTISSEYNYWGQGTQVTVSSGGGGSGGGGSGPLGVRGGG | ID NO: |
| | | GSGGGGSGGGGSEVQLVESGGGLVQAGGSLRLSCAASGRTFIGYTMGWFRQA | 1142)- |
| | | PGKEREFVASIIWRGDRTRYADSVKGRFTISGDNAKNTVYLRMNSMKPEDTA | B2D2b |
| | | VYYCAARSGSHFPSFDYWGQGTQVSVSS | |
| 1095 | Construct | MGWSCIILFLVATATGVHSEPKSSDKTHTCPPCPAPELLGGPSVFLFPPKPK | Fc hole- |
| | GGGGGGG | DTLMISRTPEVTCVVVDVSHEDPEVKFNWYVDGVEVHNAKTKPREEQYNSTY | B2G1- |
| | | RVVSVLTVLHQDWLNGKEYKCKVSNKALPAPIEKTISKAKGQPREPRVYTLP | 2×(G4S)(SEQ |
| | | PSRDELTKNQVSLTCLVKGFYPSDIAVEWESNGQPENNYKTTPPVLVSDGSF | ID NO: |
| | | TLYSKLTVDKSRWQQGNVFSCSVMHEALHNHYTQKSLSLSPGGGGGSGGGGS | 1142)- |
| | | GGGGSGGGGGGGGSQVQLVESGGGLVQAGGSLRLSCAASGRTFSNYFAGWF | MMPcs1- |
| | | RQPPGEEREFVASISWGGDRMFYTDAVKGRFTISRDNAKNTVDLQMNSLKPE | 3×(G4S)(SEQ |
| | | DTAVYYCSADRFATALYNGNGNYWGQGTQVTVSSGGGGSGGGGSGPLGVRGG | ID NO: |
| | | GGSGGGGGGGGSEVQLVESGGGLVQAGGSLRLSCAASGRTFIGYTMGWFRQ | 1142)- |
| | | APGKEREFVASIIWRGDRTRYADSVKGRFTISGDNAKNTVYLRMNSMKPEDT | B2D2b |
| | | AVYYCAARSGSHFPSFDYWGQGTQVSVSS | |
| 1096 | Construct | MGWSCIILFLVATATGVHSEPKSSDKTHTCPPCPAPELLGGPSVFLFPPKPK | Fc hole- |
| | HHHHHHH | DTLMISRTPEVTCVVVDVSHEDPEVKFNWYVDGVEVHNAKTKPREEQYNSTY | B2D2b- |
| | | RVVSVLTVLHQDWLNGKEYKCKVSNKALPAPIEKTISKAKGQPREPRVYTLP | 2×(G4S) |
| | | PSRDELTKNQVSLTCLVKGFYPSDIAVEWESNGQPENNYKTTPPVLVSDGSF | (SEQ |
| | | TLYSKLTVDKSRWQQGNVFSCSVMHEALHNHYTQKSLSLSPGGGGGSGGGGS | ID NO: |
| | | GGGGSGGGGSGGGGSEVQLVESGGGLVQAGGSLRLSCAASGRTFIGYTMGWF | 1142)- |
| | | RQAPGKEREFVASIIWRGDRTRYADSVKGRFTISGDNAKNTVYLRMNSMKPE | MMPcs1- |
| | | DTAVYYCAARSGSHFPSFDYWGQGTQVSVSSGGGGSGGGGSGPLGVRGGGGS | 3×(G4S)(SEQ |
| | | GGGGSGGGGSQVQLVESGGGLVQAGGSLRLSCAASGRTFSNYFAGWFRQPPG | ID NO: |
| | | EEREFVASISWGGDRMFYTDAVKGRFTISRDNAKNTVDLQMNSLKPEDTAVY | 1142)-B2G1 |
| | | YCSADRFATALYNGNGNYWGQGTQVTVSS | |
| 1097 | Construct | MGWSCIILFLVATATGVHSEPKSSDKTHTCPPCPAPELLGGPSVFLFPPKPK | Fc knob- |
| | | DTLMISRTPEVTCVVVDVSHEDPEVKFNWYVDGVEVHNAKTKPREEQYNSTY | MMPcs1- |
| | | RVVSVLTVLHQDWLNGKEYKCKVSNKALPAPIEKTISKAKGQPREPQVYTLP | huIL2 |
| | | PSRDELTENQVSLTCLVKGFYPSDIAVEWESNGQPENNYKTTPPVLDSDGSF | (C125S)- |
| | | FLYSWLTVDKSRWQQGNVFSCSVMHEALHNRFTQKSLSLSPGGGGGSGGGGS | VRIQRKKE |
| | | GGGGGPLGVRGGGGGSAPTSSSTKKTQLQLEHLLLDLQMILNGINNYKNPKL | KMKET |
| | | TRMLTFKFYMPKKATELKHLQCLEEELKPLEEVLNLAQSKNFHLRPRDLISN | (SEQ ID |
| | | INVIVLELKGSETTFMCEYADETATIVEFLNRWITFSQSIISTLTGSGGVRI | NO: 1139) |
| | | QRKKEKMKET | |
| 1098 | Construct | MGWSCIILFLVATATGVHSEPKSSDKTHTCPPCPAPELLGGPSVFLFPPKPK | Fc knob- |
| | JJJJJJJ | DTLMISRTPEVTCVVVDVSHEDPEVKFNWYVDGVEVHNAKTKPREEQYNSTY | MMPcs1- |
| | | RVVSVLTVLHQDWLNGKEYKCKVSNKALPAPIEKTISKAKGQPREPQVYTLP | huIL2 |
| | | PSRDELTENQVSLTCLVKGFYPSDIAVEWESNGQPENNYKTTPPVLDSDGSF | (C125S)- |
| | | FLYSWLTVDKSRWQQGNVFSCSVMHEALHNRFTQKSLSLSPGGGGGSGGGGS | KLWVLPK |
| | | GGGGGPLGVRGGGGGSAPTSSSTKKTQLQLEHLLLDLQMILNGINNYKNPKL | (SEQ ID |
| | | TRMLTFKFYMPKKATELKHLQCLEEELKPLEEVLNLAQSKNFHLRPRDLISN | NO: 200) |
| | | INVIVLELKGSETTFMCEYADETATIVEFLNRWITFSQSIISTLTGSGGKLW | |
| | | VLPK | |
| 1099 | Construct | MGWSCIILFLVATATGVHSEPKSSDKTHTCPPCPAPELLGGPSVFLFPPKPK | Fc knob- |
| | KKKKKKK | DTLMISRTPEVTCVVVDVSHEDPEVKFNWYVDGVEVHNAKTKPREEQYNSTY | MMPcs1- |
| | | RVVSVLTVLHQDWLNGKEYKCKVSNKALPAPIEKTISKAKGQPREPQVYTLP | huIL2 |
| | | PSRDELTENQVSLTCLVKGFYPSDIAVEWESNGQPENNYKTTPPVLDSDGSF | (C125S)- |
| | | FLYSWLTVDKSRWQQGNVFSCSVMHEALHNRFTQKSLSLSPGGGGGSGGGGS | LRELHLDN |
| | | GGGGGPLGVRGGGGGSAPTSSSTKKTQLQLEHLLLDLQMILNGINNYKNPKL | N (SEQ ID |
| | | TRMLTFKFYMPKKATELKHLQCLEEELKPLEEVLNLAQSKNFHLRPRDLISN | NO: 188) |
| | | INVIVLELKGSETTFMCEYADETATIVEFLNRWITFSQSIISTLTGSGGLRE | |
| | | LHLDNN | |
| 1100 | Construct | EPKSSDKTHTCPPCPAPELLGGPSVFLFPPKPKDTLMISRTPEVTCVVVDVS | Fc knob- |
| | LLLLLLL | HEDPEVKFNWYVDGVEVHNAKTKPREEQYNSTYRVVSVLTVLHQDWLNGKEY | MMPcs1- |
| | | KCKVSNKALPAPIEKTISKAKGQPREPQVYTLPPSRDELTENQVSLTCLVKG | huIL2 |
| | | FYPSDIAVEWESNGQPENNYKTTPPVLDSDGSFFLYSWLTVDKSRWQQGNVF | (C125S)- |
| | | SCSVMHEALHNRFTQKSLSLSPGGGGGSGGGGSGGGGGPLGVRGGGGGSAPT | GWSHW |
| | | SSSTKKTQLQLEHLLLDLQMILNGINNYKNPKLTRMLTFKFYMPKKATELKH | (SEQ ID |
| | | LQCLEEELKPLEEVLNLAQSKNFHLRPRDLISNINVIVLELKGSETTFMCEY | NO: 1167) |
| | | ADETATIVEFLNRWITFSQSIISTLTGSGGGGWSHW | |
| 1101 | Construct | EPKSSDKTHTCPPCPAPELLGGPSVFLFPPKPKDTLMISRTPEVTCVVVDVS | Fc knob- |
| | MMMMMM | HEDPEVKFNWYVDGVEVHNAKTKPREEQYNSTYRVVSVLTVLHQDWLNGKEY | MMPcs1- |
| | M | KCKVSNKALPAPIEKTISKAKGQPREPQVYTLPPSRDELTENQVSLTCLVKG | huIL2 |
| | | FYPSDIAVEWESNGQPENNYKTTPPVLDSDGSFFLYSWLTVDKSRWQQGNVF | (C125S) |
| | | SCSVMHEALHNRFTQKSLSLSPGGGGGSGGGGSGGGGGPLGVRGGGGGSAPT | |
| | | SSSTKKTQLQLEHLLLDLQMILNGINNYKNPKLTRMLTFKFYMPKKATELKH | |
| | | LQCLEEELKPLEEVLNLAQSKNFHLRPRDLISNINVIVLELKGSETTFMCEY | |
| | | ADETATIVEFLNRWITFSQSIISTLT | |
| 1102 | Construct | MGWSCIILFLVATATGVHSEPKSSDKTHTCPPCPAPELLGGPSVFLFPPKPK | Fc knob- |
| | NNNNNNN | DTLMISRTPEVTCVVVDVSHEDPEVKFNWYVDGVEVHNAKTKPREEQYNSTY | MMPcs1- |
| | | RVVSVLTVLHQDWLNGKEYKCKVSNKALPAPIEKTISKAKGQPREPQVYTLP | huIL2 |
| | | PSRDELTENQVSLTCLVKGFYPSDIAVEWESNGQPENNYKTTPPVLDSDGSF | (C125S) |
| | | FLYSWLTVDKSRWQQGNVFSCSVMHEALHNRFTQKSLSLSPGGGGGSGGGGS | |
| | | GPLGVRGGGGGSGGGGAPTSSSTKKTQLQLEHLLLDLQMILNGINNYKNPKL | |
| | | TRMLTFKFYMPKKATELKHLQCLEEELKPLEEVLNLAQSKNFHLRPRDLISN | |
| | | INVIVLELKGSETTFMCEYADETATIVEFLNRWITFSQSIISTLT | |
| 1103 | Construct | MGWSCIILFLVATATGVHSEPKSSDKTHTCPPCPAPELLGGPSVFLFPPKPK | Fc knob- |
| | 0000000 | DTLMISRTPEVTCVVVDVSHEDPEVKFNWYVDGVEVHNAKTKPREEQYNSTY | MMPcs1- |
| | | RVVSVLTVLHQDWLNGKEYKCKVSNKALPAPIEKTISKAKGQPREPQVYTLP | huIL2 |
| | | PSRDELTENQVSLTCLVKGFYPSDIAVEWESNGQPENNYKTTPPVLDSDGSF | (C125S)- |
| | | FLYSWLTVDKSRWQQGNVFSCSVMHEALHNRFTQKSLSLSPGGGGGSGGGGS | KLWVLPK |
| | | GPLGVRGGGGGSGGGGAPTSSSTKKTQLQLEHLLLDLQMILNGINNYKNPKL | (SEQ ID |
| | | TRMLTFKFYMPKKATELKHLQCLEEELKPLEEVLNLAQSKNFHLRPRDLISN | NO: 200) |
| | | INVIVLELKGSETTFMCEYADETATIVEFLNRWITFSQSIISTLTGSGGKLW | |
| | | VLPK | |
| 1104 | Construct | MGWSCIILFLVATATGVHSEPKSSDKTHTCPPCPAPELLGGPSVFLFPPKPK | Fc knob- |
| | PPPPPPP | DTLMISRTPEVTCVVVDVSHEDPEVKFNWYVDGVEVHNAKTKPREEQYNSTY | MMPcs1- |
| | | RVVSVLTVLHQDWLNGKEYKCKVSNKALPAPIEKTISKAKGQPREPQVYTLP | huIL2 |
| | | PSRDELTENQVSLTCLVKGFYPSDIAVEWESNGQPENNYKTTPPVLDSDGSF | (C125S)- |
| | | FLYSWLTVDKSRWQQGNVFSCSVMHEALHNRFTQKSLSLSPGGGGGSGGGGS | LRELHLDN |
| | | GPLGVRGGGGGSGGGGAPTSSSTKKTQLQLEHLLLDLQMILNGINNYKNPKL | N (SEQ ID |
| | | TRMLTFKFYMPKKATELKHLQCLEEELKPLEEVLNLAQSKNFHLRPRDLISN | NO: 188) |
| | | INVIVLELKGSETTFMCEYADETATIVEFLNRWITFSQSIISTLTGSGGLRE | |
| | | LHLDNN | |
| 1105 | Construct | MGWSCIILFLVATATGVHSVRIQRKKEKMKETGGGGSEPKSSDKTHTCPPCP | VRIQRKKE |
| | QQQQQQQ | APELLGGPSVFLFPPKPKDTLMISRTPEVTCVVVDVSHEDPEVKFNWYVDGV | KMKET |
| | | EVHNAKTKPREEQYNSTYRVVSVLTVLHQDWLNGKEYKCKVSNKALPAPIEK | (SEQ ID |
| | | TISKAKGQPREPQVYTLPPSRDELTENQVSLTCLVKGFYPSDIAVEWESNGQ | NO: 1139)- |
| | | PENNYKTTPPVLDSDGSFFLYSWLTVDKSRWQQGNVFSCSVMHEALHNRFTQ | Fc knob- |
| | | KSLSLSPGGGGSGGGGGPLGVRGGGGGSAPTSSSTKKTQLQLEHLLLDLQMI | MMPcs1- |
| | | LNGINNYKNPKLTRMLTFKFYMPKKATELKHLQCLEEELKPLEEVLNLAQSK | huIL2 |
| | | NFHLRPRDLISNINVIVLELKGSETTFMCEYADETATIVEFLNRWITFSQSI | (C125S) |
| | | ISTLT | |
| 1106 | Construct | MGWSCIILFLVATATGVHSLRELHLDNNGGGGSEPKSSDKTHTCPPCPAPEL | LRELHLDN |
| | RRRRRRR | LGGPSVFLFPPKPKDTLMISRTPEVTCVVVDVSHEDPEVKFNWYVDGVEVHN | N (SEQ ID |
| | | AKTKPREEQYNSTYRVVSVLTVLHQDWLNGKEYKCKVSNKALPAPIEKTISK | NO: 188)-Fc |
| | | AKGQPREPQVYTLPPSRDELTENQVSLTCLVKGFYPSDIAVEWESNGQPENN | knob- |
| | | YKTTPPVLDSDGSFFLYSWLTVDKSRWQQGNVFSCSVMHEALHNRFTQKSLS | MMPcs1- |
| | | LSPGGGGGGGGGPLGVRGGGGGSAPTSSSTKKTQLQLEHLLLDLQMILNGI | huIL2 |
| | | NNYKNPKLTRMLTFKFYMPKKATELKHLQCLEEELKPLEEVLNLAQSKNFHL | (C125S) |
| | | RPRDLISNINVIVLELKGSETTFMCEYADETATIVEFLNRWITFSQSIISTL | |
| | | T | |
| 1107 | Construct | MGWSCIILFLVATATGVHSKLWVLPKGGGGSEPKSSDKTHTCPPCPAPELLG | KLWVLPK |
| | SSSSSSS | GPSVFLFPPKPKDTLMISRTPEVTCVVVDVSHEDPEVKFNWYVDGVEVHNAK | (SEQ ID |
| | | TKPREEQYNSTYRVVSVLTVLHQDWLNGKEYKCKVSNKALPAPIEKTISKAK | NO: 200)-Fc |
| | | GQPREPQVYTLPPSRDELTENQVSLTCLVKGFYPSDIAVEWESNGQPENNYK | knob- |
| | | TTPPVLDSDGSFFLYSWLTVDKSRWQQGNVFSCSVMHEALHNRFTQKSLSLS | MMPcs1- |
| | | PGGGGSGGGGGPLGVRGGGGGSAPTSSSTKKTQLQLEHLLLDLQMILNGINN | huIL2 |
| | | YKNPKLTRMLTFKFYMPKKATELKHLQCLEEELKPLEEVLNLAQSKNFHLRP | (C125S) |
| | | RDLISNINVIVLELKGSETTFMCEYADETATIVEFLNRWITFSQSIISTLT | |
| 1108 | Construct | MGWSCIILFLVATATGVHSGGWSHWGGGGSEPKSSDKTHTCPPCPAPELLGG | fibronectin- |
| | TTTTTTT | PSVFLFPPKPKDTLMISRTPEVTCVVVDVSHEDPEVKFNWYVDGVEVHNAKT | Fc knob- |
| | | KPREEQYNSTYRVVSVLTVLHQDWLNGKEYKCKVSNKALPAPIEKTISKAKG | MMPcs1- |
| | | QPREPQVYTLPPSRDELTENQVSLTCLVKGFYPSDIAVEWESNGQPENNYKT | huIL2 |
| | | TPPVLDSDGSFFLYSWLTVDKSRWQQGNVFSCSVMHEALHNRFTQKSLSLSP | (C125S) |
| | | GGGGSGGGGGPLGVRGGGGGSAPTSSSTKKTQLQLEHLLLDLQMILNGINNY | |
| | | KNPKLTRMLTFKFYMPKKATELKHLQCLEEELKPLEEVLNLAQSKNFHLRPR | |
| | | DLISNINVIVLELKGSETTFMCEYADETATIVEFLNRWITFSQSIISTLT | |
| 1109 | Construct | EPKSSDKTHTCPPCPAPELLGGPSVFLFPPKPKDTLMISRTPEVTCVVVDVS | Fc knob- |
| | UUUUUUU | HEDPEVKFNWYVDGVEVHNAKTKPREEQYNSTYRVVSVLTVLHQDWLNGKEY | MMPscr- |
| | | KCKVSNKALPAPIEKTISKAKGQPREPQVYTLPPSRDELTENQVSLTCLVKG | huIL2 |
| | | FYPSDIAVEWESNGQPENNYKTTPPVLDSDGSFFLYSWLTVDKSRWQQGNVF | (C125S) |
| | | SCSVMHEALHNRFTQKSLSLSPGGGGSGGGGGVRLGPGGGGGSAPTSSSTKK | |
| | | TQLQLEHLLLDLQMILNGINNYKNPKLTRMLTFKFYMPKKATELKHLQCLEE | |
| | | ELKPLEEVLNLAQSKNFHLRPRDLISNINVIVLELKGSETTFMCEYADETAT | |
| | | IVEFLNRWITFSQSIISTLT | |
| 1110 | Construct | EPKSSDKTHTCPPCPAPELLGGPSVFLFPPKPKDTLMISRTPEVTCVVVDVS | Fc knob- |
| | VVvvvVV | HEDPEVKFNWYVDGVEVHNAKTKPREEQYNSTYRVVSVLTVLHQDWLNGKEY | MMPscr- |
| | | KCKVSNKALPAPIEKTISKAKGQPREPQVYTLPPSRDELTENQVSLTCLVKG | huIL2 |
| | | FYPSDIAVEWESNGQPENNYKTTPPVLDSDGSFFLYSWLTVDKSRWQQGNVF | (C125S)- |
| | | SCSVMHEALHNRFTQKSLSLSPGGGGGSGGGGSGGGGGVRLGPGGGGGSAPT | VRIQRKKE |
| | | SSSTKKTQLQLEHLLLDLQMILNGINNYKNPKLTRMLTFKFYMPKKATELKH | KMKET |
| | | LQCLEEELKPLEEVLNLAQSKNFHLRPRDLISNINVIVLELKGSETTFMCEY | (SEQ ID |
| | | ADETATIVEFLNRWITFSQSIISTLTGSGGVRIQRKKEKMKET | NO: 1139) |
| 1111 | Construct | EPKSSDKTHTCPPCPAPELLGGPSVFLFPPKPKDTLMISRTPEVTCVVVDVS | Fc knob- |
| | WWWWWW | HEDPEVKFNWYVDGVEVHNAKTKPREEQYNSTYRVVSVLTVLHQDWLNGKEY | MMPscr- |
| | W | KCKVSNKALPAPIEKTISKAKGQPREPQVYTLPPSRDELTENQVSLTCLVKG | huIL2 |
| | | FYPSDIAVEWESNGQPENNYKTTPPVLDSDGSFFLYSWLTVDKSRWQQGNVF | (C125S)- |
| | | SCSVMHEALHNRFTQKSLSLSPGGGGGSGGGGSGGGGGVRLGPGGGGGSAPT | KLWVLPK |
| | | SSSTKKTQLQLEHLLLDLQMILNGINNYKNPKLTRMLTFKFYMPKKATELKH | (SEQ ID |
| | | LQCLEEELKPLEEVLNLAQSKNFHLRPRDLISNINVIVLELKGSETTFMCEY | NO: 200) |
| | | ADETATIVEFLNRWITFSQSIISTLTGSGGKLWVLPK | |
| 1112 | Construct | EPKSSDKTHTCPPCPAPELLGGPSVFLFPPKPKDTLMISRTPEVTCVVVDVS | Fc knob |
| | XXXXXXX | HEDPEVKFNWYVDGVEVHNAKTKPREEQYNSTYRVVSVLTVLHQDWLNGKEY | MMPscr- |
| | | KCKVSNKALPAPIEKTISKAKGQPREPQVYTLPPSRDELTENQVSLTCLVKG | huIL2 |
| | | FYPSDIAVEWESNGQPENNYKTTPPVLDSDGSFFLYSWLTVDKSRWQQGNVF | (C125S)- |
| | | SCSVMHEALHNRFTQKSLSLSPGGGGGSGGGGSGGGGGVRLGPGGGGGSAPT | LRELHLDN |
| | | SSSTKKTQLQLEHLLLDLQMILNGINNYKNPKLTRMLTFKFYMPKKATELKH | N (SEQ ID |
| | | LQCLEEELKPLEEVLNLAQSKNFHLRPRDLISNINVIVLELKGSETTFMCEY | NO: 188) |
| | | ADETATIVEFLNRWITFSQSIISTLTGSGGLRELHLDNN | |
| 1113 | Construct | EPKSSDKTHTCPPCPAPELLGGPSVFLFPPKPKDTLMISRTPEVTCVVVDVS | Fc knob- |
| | | HEDPEVKFNWYVDGVEVHNAKTKPREEQYNSTYRVVSVLTVLHQDWLNGKEY | MMPscr- |
| | | KCKVSNKALPAPIEKTISKAKGQPREPQVYTLPPSRDELTENQVSLTCLVKG | huIL2 |
| | | FYPSDIAVEWESNGQPENNYKTTPPVLDSDGSFFLYSWLTVDKSRWQQGNVF | (C125S)- |
| | | SCSVMHEALHNRFTQKSLSLSPGGGGGSGGGGSGGGGGVRLGPGGGGGSAPT | GWSHW |
| | | SSSTKKTQLQLEHLLLDLQMILNGINNYKNPKLTRMLTFKFYMPKKATELKH | (SEQ ID |
| | | LQCLEEELKPLEEVLNLAQSKNFHLRPRDLISNINVIVLELKGSETTFMCEY | NO: 1167) |
| | | ADETATIVEFLNRWITFSQSIISTLTGSGGGGWSHW | |
| 1114 | Construct | EPKSSDKTHTCPPCPAPEAAGGPSVFLFPPKPKDTLMISRTPEVTCVVVDVS | Fc hole- |
| | ZZZZZZZ | HEDPEVKFNWYVDGVEVHNAKTKPREEQYNSTYRVVSVLTVLHQDWLNGKEY | B2G1 |
| | | KCKVSNKALPAPIEKTISKAKGQPREPQVYTLPPSREEMTKNQVSLSCAVKG | |
| | | FYPSDIAVEWESNGQPENNYKTTPPVLDSDGSFFLVSKLTVDKSRWQQGNVF | |
| | | SCSVMHEALHNHYTQKSLSLSPGGGGGSGGGGSGGGGSGGGGSGGGGSQVQL | |
| | | VESGGGLVQAGGSLRLSCAASGRTFSNYFAGWFRQPPGEEREFVASISWGGD | |
| | | RMFYTDAVKGRFTISRDNAKNTVDLQMNSLKPEDTAVYYCSADRFATALYNG | |
| | | NGNYWGQGTQVTVSS | |
| 1115 | Construct | EPKSSDKTHTCPPCPAPEAAGGPSVFLFPPKPKDTLMISRTPEVTCVVVDVS | Fc hole- |
| | AAAAAAAA | HEDPEVKFNWYVDGVEVHNAKTKPREEQYNSTYRVVSVLTVLHQDWLNGKEY | B2G1- |
| | | KCKVSNKALPAPIEKTISKAKGQPREPQVYTLPPSREEMTKNQVSLSCAVKG | 2×(G4S)(SEQ |
| | | FYPSDIAVEWESNGQPENNYKTTPPVLDSDGSFFLVSKLTVDKSRWQQGNVF | ID NO: |
| | | SCSVMHEALHNHYTQKSLSLSPGGGGGSGGGGSGGGGSGGGGSGGGGSQVQL | 1142)- |
| | | VESGGGLVQAGGSLRLSCAASGRTFSNYFAGWFRQPPGEEREFVASISWGGD | MMPcs1- |
| | | RMFYTDAVKGRFTISRDNAKNTVDLQMNSLKPEDTAVYYCSADRFATALYNG | 3×(G4S)(SEQ |
| | | NGNYWGQGTQVTVSSGGGGSGGGGSGPLGVRGGGGSGGGGSGGGGSEVQLVE | ID NO: |
| | | SGGGLVQAGGSLRLSCAASGRTFIGYTMGWFRQAPGKEREFVASIIWRGDRT | 1142)- |
| | | RYADSVKGRFTISGDNAKNTVYLRMNSMKPEDTAVYYCAARSGSHFPSFDYW | B2D2b |
| | | GQGTQVSVSS | |
| 1116 | Construct | EPKSSDKTHTCPPCPAPEAAGGPSVFLFPPKPKDTLMISRTPEVTCVVVDVS | Fc knob- |
| | BBBBBBBB | HEDPEVKFNWYVDGVEVHNAKTKPREEQYNSTYRVVSVLTVLHQDWLNGKEY | MMPcs1- |
| | | KCKVSNKALPAPIEKTISKAKGQPREPQVYTLPPSREEMTKNQVSLWCLVKG | huIL2 |
| | | FYPSDIAVEWESNGQPENNYKTTPPVLDSDGSFFLYSKLTVDKSRWQQGNVF | (C125S) |
| | | SCSVMHEALHNRFTQKSLSLSPGGGGSGGGGGPLGVRGGGGGSAPTSSSTKK | |
| | | TQLQLEHLLLDLQMILNGINNYKNPKLTRMLTFKFYMPKKATELKHLQCLEE | |
| | | ELKPLEEVLNLAQSKNFHLRPRDLISNINVIVLELKGSETTFMCEYADETAT | |
| | | IVEFLNRWITFSQSIISTLT | |
| 1117 | Construct | EPKSSDKTHTCPPCPAPEAAGGPSVFLFPPKPKDTLMISRTPEVTCVVVDVS | Fc knob |
| | CCCCCCCC | HEDPEVKFNWYVDGVEVHNAKTKPREEQYNSTYRVVSVLTVLHQDWLNGKEY | MMPcs1- |
| | | KCKVSNKALPAPIEKTISKAKGQPREPQVYTLPPSREEMTKNQVSLWCLVKG | huIL2 |
| | | FYPSDIAVEWESNGQPENNYKTTPPVLDSDGSFFLYSKLTVDKSRWQQGNVF | (C125S)- |
| | | SCSVMHEALHNRFTQKSLSLSPGGGGGSGGGGSGGGGGPLGVRGGGGGSAPT | LRELHLDN |
| | | SSSTKKTQLQLEHLLLDLQMILNGINNYKNPKLTRMLTFKFYMPKKATELKH | N (SEQ ID |
| | | LQCLEEELKPLEEVLNLAQSKNFHLRPRDLISNINVIVLELKGSETTFMCEY | NO: 188) |
| | | ADETATIVEFLNRWITFSQSIISTLTGSGGLRELHLDNN | |
| 1118 | Construct | EPKSSDKTHTCPPCPAPEAAGGPSVFLFPPKPKDTLMISRTPEVTCVVVDVS | Fc knob- |
| | DDDDDDDD | HEDPEVKFNWYVDGVEVHNAKTKPREEQYNSTYRVVSVLTVLHQDWLNGKEY | MMPcs1- |
| | | KCKVSNKALPAPIEKTISKAKGQPREPQVYTLPPSREEMTKNQVSLWCLVKG | huIL2 |
| | | FYPSDIAVEWESNGQPENNYKTTPPVLDSDGSFFLYSKLTVDKSRWQQGNVF | (C125S)- |
| | | SCSVMHEALHNRFTQKSLSLSPGGGGGSGGGGSGGGGGPLGVRGGGGGSAPT | GWSHW |
| | | SSSTKKTQLQLEHLLLDLQMILNGINNYKNPKLTRMLTFKFYMPKKATELKH | (SEQ ID |
| | | LQCLEEELKPLEEVLNLAQSKNFHLRPRDLISNINVIVLELKGSETTFMCEY | NO: 1167) |
| | | ADETATIVEFLNRWITFSQSIISTLTGSGGGGWSHW | |
| 1119 | Construct | EPKSSDKTHTCPPCPAPEAAGGPSVFLFPPKPKDTLMISRTPEVTCVVVDVS | Fc knob- |
| | EEEEEEEE | HEDPEVKFNWYVDGVEVHNAKTKPREEQYNSTYRVVSVLTVLHQDWLNGKEY | MMPcs1- |
| | | KCKVSNKALPAPIEKTISKAKGQPREPQVYTLPPSREEMTKNQVSLWCLVKG | huIL2 |
| | | FYPSDIAVEWESNGQPENNYKTTPPVLDSDGSFFLYSKLTVDKSRWQQGNVF | (C125S)- |
| | | SCSVMHEALHNRFTQKSLSLSPGGGGGSGGGGSGGGGGPLGVRGGGGGSAPT | VRIQRKKE |
| | | SSSTKKTQLQLEHLLLDLQMILNGINNYKNPKLTRMLTFKFYMPKKATELKH | KMKET |
| | | LQCLEEELKPLEEVLNLAQSKNFHLRPRDLISNINVIVLELKGSETTFMCEY | (SEQ ID |
| | | ADETATIVEFLNRWITFSQSIISTLTGSGGVRIQRKKEKMKET | NO: 1139) |
| 1120 | Construct | ITCPPPMSVEHADIWVKSYSLYSRERYICNSGFKRKAGTSSLTECVLNKATN | hu IL15Ra |
| | TTTT | VAHWTTPSLKCIRDPALVHQRPAPPSGGSGGGGSGGGSGGGGSLQNWVNVIS | (1-77)- |
| | | DLKKIEDLIQSMHIDATLYTESDVHPSCKVTAMKCFLLELQVISLESGDASI | linker-hu |
| | | HDTVENLIILANNSLSSNGNVTESGCKECEELEEKNIKEFLQSFVHIVQMFI | IL15- |
| | | NTSSGGGGPLGVRGGGGGSGGGGSGGGGSGGGGSAVNGTSQFTCFYNSRANI | (SG3)(SEQ |
| | | SCVWSQDGALQDTSCQVHAWPDRRRWNQTCELLPVSQASWACNLILGAPDSQ | ID NO: |
| | | KLTTVDIVTLRVLCREGVRWRVMAIQDFKPFENLRLMAPISLQVVHVETHRC | 1158)- |
| | | NISWEISQASHYFERHLEFEARTLSPGHTWEEAPLLTLKQKQEWICLETLTP | GPLGVRG |
| | | DTQYEFQVRVKPLQGEFTTWSPWSQPLAFRTKPAALGKDTGGGGSGEPKSSD | (SEQ ID |
| | | KTHTCPPCPAPEAAGGPSVFLFPPKPKDTLMISRTPEVTCVVVDVSHEDPEV | NO: 80)- |
| | | KFNWYVDGVEVHNAKTKPREEQYNSTYRVVSVLTVLHQDWLNGKEYKCKVSN | 4×(G4S)(SEQ |
| | | KALPAPIEKTISKAKGQPREPQVYTLPPSRDELTKNQVSLTCLVKGFYPSDI | ID NO: |
| | | AVEWESNGQPENNYKTTPPVLDSDGSFFLYSKLTVDKSRWQQGNVFSCSVMH | 1142)-IL2Rb |
| | | EALHNHYTQKSLSLSPG | (1-214)- |
| | | | (G4SG) |
| | | | (SEQ ID |
| | | | NO: 1162)- |
| | | | Hu IgG1 Fc |
| 1121 | Construct | ITCPPPMSVEHADIWVKSYSLYSRERYICNSGFKRKAGTSSLTECVLNKATN | hu IL 15Ra |
| | QQQQ | VAHWTTPSLKCIRDPALVHQRPAPPSGGSGGGGSGGGSGGGGSLQNWVNVIS | (1-77)- |
| | | DLKKIEDLIQSMHIDATLYTESDVHPSCKVTAMKCFLLELQVISLESGDASI | linker-hu |
| | | HDTVENLIILANNSLSSNGNVTESGCKECEELEEKNIKEFLQSFVHIVQMFI | IL15-(SG3) |
| | | NTSSGGGGPLGVRGGGGSGGWSHWGGGGSGGGGSAVNGTSQFTCFYNSRANI | (SEQ ID |
| | | SCVWSQDGALQDTSCQVHAWPDRRRWNQTCELLPVSQASWACNLILGAPDSQ | NO: 1158)- |
| | | KLTTVDIVTLRVLCREGVRWRVMAIQDFKPFENLRLMAPISLQVVHVETHRC | GPLGVRG |
| | | NISWEISQASHYFERHLEFEARTLSPGHTWEEAPLLTLKQKQEWICLETLTP | (SEQ ID |
| | | DTQYEFQVRVKPLQGEFTTWSPWSQPLAFRTKPAALGKDTGGGGSGEPKSSD | NO: 80)- |
| | | KTHTCPPCPAPEAAGGPSVFLFPPKPKDTLMISRTPEVTCVVVDVSHEDPEV | (G3S)- |
| | | KFNWYVDGVEVHNAKTKPREEQYNSTYRVVSVLTVLHQDWLNGKEYKCKVSN | GGWSHW |
| | | KALPAPIEKTISKAKGQPREPQVYTLPPSRDELTKNQVSLTCLVKGFYPSDI | (SEQ ID |
| | | AVEWESNGQPENNYKTTPPVLDSDGSFFLYSKLTVDKSRWQQGNVFSCSVMH | NO: 653)- |
| | | EALHNHYTQKSLSLSPG | 2×(G4S)(SEQ |
| | | | ID NO: |
| | | | 1142)-IL2Rb |
| | | | (1-214)- |
| | | | (G4SG) |
| | | | (SEQ ID |
| | | | NO: 1162)- |
| | | | Hu IgG1 Fc |
| 1122 | Construct | MGWSCIILFLVATATGVHSITCPPPMSVEHADIWVKSYSLYSRERYICNSGF | hu IL15Ra |
| | UUUU | KRKAGTSSLTECVLNKATNVAHWTTPSLKCIRDPALVHQRPAPPSGGSGGGG | (1-77)- |
| | | SGGGSGGGGSLQNWVNVISDLKKIEDLIQSMHIDATLYTESDVHPSCKVTAM | linker-hu |
| | | KCFLLELQVISLESGDASIHDTVENLIILANNSLSSNGNVTESGCKECEELE | IL15-(SG3) |
| | | EKNIKEFLQSFVHIVQMFINTSSGGGGVRLGPGGGGSGGWSHWGGGGSGGGG | (SEQ ID |
| | | SAVNGTSQFTCFYNSRANISCVWSQDGALQDTSCQVHAWPDRRRWNQTCELL | NO: 1158)- |
| | | PVSQASWACNLILGAPDSQKLTTVDIVTLRVLCREGVRWRVMAIQDFKPFEN | MMPscr- |
| | | LRLMAPISLQVVHVETHRCNISWEISQASHYFERHLEFEARTLSPGHTWEEA | (G3S)- |
| | | PLLTLKQKQEWICLETLTPDTQYEFQVRVKPLQGEFTTWSPWSQPLAFRTKP | GGWSHW |
| | | AALGKDTGGGGSGEPKSSDKTHTCPPCPAPEAAGGPSVFLFPPKPKDTLMIS | (SEQ ID |
| | | RTPEVTCVVVDVSHEDPEVKFNWYVDGVEVHNAKTKPREEQYNSTYRVVSVL | NO: 653)- |
| | | TVLHQDWLNGKEYKCKVSNKALPAPIEKTISKAKGQPREPQVYTLPPSRDEL | 2×(G4S)(SEQ |
| | | TKNQVSLTCLVKGFYPSDIAVEWESNGQPENNYKTTPPVLDSDGSFFLYSKL | ID NO: |
| | | TVDKSRWQQGNVFSCSVMHEALHNHYTQKSLSLSPG | 1142)-IL2Rb |
| | | | (1-214)- |
| | | | (G4SG) |
| | | | (SEQ ID |
| | | | NO: 1162)- |
| | | | Hu IgG1 Fc |
| 1123 | Construct | MGWSCIILFLVATATGVHSITCPPPMSVEHADIWVKSYSLYSRERYICNSGF | hu IL 15Ra |
| | RRRR | KRKAGTSSLTECVLNKATNVAHWTTPSLKCIRDPALVHQRPAPPSGGSGGGG | (1-77)- |
| | | SGGGSGGGGSLQNWVNVISDLKKIEDLIQSMHIDATLYTESDVHPSCKVTAM | linker-hu |
| | | KCFLLELQVISLESGDASIHDTVENLIILANNSLSSNGNVTESGCKECEELE | IL15-(SG3) |
| | | EKNIKEFLQSFVHIVQMFINTSSGGGGPLGVRGGGSLRELHLDNNGGGGSGG | (SEQ ID |
| | | GGSAVNGTSQFTCFYNSRANISCVWSQDGALQDTSCQVHAWPDRRRWNQTCE | NO: 1158)- |
| | | LLPVSQASWACNLILGAPDSQKLTTVDIVTLRVLCREGVRWRVMAIQDFKPF | GPLGVRG |
| | | ENLRLMAPISLQVVHVETHRCNISWEISQASHYFERHLEFEARTLSPGHTWE | (SEQ ID |
| | | EAPLLTLKQKQEWICLETLTPDTQYEFQVRVKPLQGEFTTWSPWSQPLAFRT | NO: 80)- |
| | | KPAALGKDTGGGGSGEPKSSDKTHTCPPCPAPEAAGGPSVFLFPPKPKDTLM | (G3S)- |
| | | ISRTPEVTCVVVDVSHEDPEVKFNWYVDGVEVHNAKTKPREEQYNSTYRVVS | LRELHLDN |
| | | VLTVLHQDWLNGKEYKCKVSNKALPAPIEKTISKAKGQPREPQVYTLPPSRD | N (SEQ ID |
| | | ELTKNQVSLTCLVKGFYPSDIAVEWESNGQPENNYKTTPPVLDSDGSFFLYS | NO: 188)- |
| | | KLTVDKSRWQQGNVFSCSVMHEALHNHYTQKSLSLSPG | 2×(G4S)(SEQ |
| | | | ID NO: |
| | | | 1142)-IL2Rb |
| | | | (1-214)- |
| | | | (G4SG) |
| | | | (SEQ ID |
| | | | NO: 1162)- |
| | | | Hu IgG1 Fc |
| 1124 | Construct | ITCPPPMSVEHADIWVKSYSLYSRERYICNSGFKRKAGTSSLTECVLNKATN | hu IL 15Ra |
| | VVVV | VAHWTTPSLKCIRDPALVHQRPAPPSGGSGGGGSGGGSGGGGSLQNWVNVIS | (1-77)- |
| | | DLKKIEDLIQSMHIDATLYTESDVHPSCKVTAMKCFLLELQVISLESGDASI | linker-hu |
| | | HDTVENLIILANNSLSSNGNVTESGCKECEELEEKNIKEFLQSFVHIVQMFI | IL15-(SG3) |
| | | NTSSGGGGVRLGPGGGSLRELHLDNNGGGGSGGGGSAVNGTSQFTCFYNSRA | (SEQ ID |
| | | NISCVWSQDGALQDTSCQVHAWPDRRRWNQTCELLPVSQASWACNLILGAPD | NO: 1158)- |
| | | SQKLTTVDIVTLRVLCREGVRWRVMAIQDFKPFENLRLMAPISLQVVHVETH | MMPscr- |
| | | RCNISWEISQASHYFERHLEFEARTLSPGHTWEEAPLLTLKQKQEWICLETL | (G3S)- |
| | | TPDTQYEFQVRVKPLQGEFTTWSPWSQPLAFRTKPAALGKDTGGGGSGEPKS | LRELHLDN |
| | | SDKTHTCPPCPAPEAAGGPSVFLFPPKPKDTLMISRTPEVTCVVVDVSHEDP | N (SEQ ID |
| | | EVKFNWYVDGVEVHNAKTKPREEQYNSTYRVVSVLTVLHQDWLNGKEYKCKV | NO: 188)- |
| | | SNKALPAPIEKTISKAKGQPREPQVYTLPPSRDELTKNQVSLTCLVKGFYPS | 2×(G4S)(SEQ |
| | | DIAVEWESNGQPENNYKTTPPVLDSDGSFFLYSKLTVDKSRWQQGNVFSCSV | ID NO: |
| | | MHEALHNHYTQKSLSLSPG | 1142)-IL2Rb |
| | | | (1-214)- |
| | | | (G4SG) |
| | | | (SEQ ID |
| | | | NO: 1162)- |
| | | | Hu IgG1 Fc |
| 1125 | Construct | EPKSSDKTHTCPPCPAPELLGGPSVFLFPPKPKDTLMISRTPEVTCVVVDVS | Fc hole- |
| | WWWW | HEDPEVKFNWYVDGVEVHNAKTKPREEQYNSTYRVVSVLTVLHQDWLNGKEY | 5×(G4S)(SEQ |
| | | KCKVSNKALPAPIEKTISKAKGQPREPRVYTLPPSRDELTKNQVSLTCLVKG | ID NO: |
| | | FYPSDIAVEWESNGQPENNYKTTPPVLVSDGSFTLYSKLTVDKSRWQQGNVF | 1142)- |
| | | SCSVMHEALHNHYTQKSLSLSPGGGGGSGGGGSGGGGSGGGGSGGGGSAVNG | IL2Rb (1- |
| | | TSQFTCFYNSRANISCVWSQDGALQDTSCQVHAWPDRRRWNQTCELLPVSQA | 213) |
| | | SWACNLILGAPDSQKLTTVDIVTLRVLCREGVRWRVMAIQDFKPFENLRLMA | |
| | | PISLQVVHVETHRCNISWEISQASHYFERHLEFEARTLSPGHTWEEAPLLTL | |
| | | KQKQEWICLETLTPDTQYEFQVRVKPLQGEFTTWSPWSQPLAFRTKPAALGK | |
| | | D | |
| 1126 | Construct | EPKSSDKTHTCPPCPAPELLGGPSVFLFPPKPKDTLMISRTPEVTCVVVDVS | Fc hole- |
| | XXXX | HEDPEVKFNWYVDGVEVHNAKTKPREEQYNSTYRVVSVLTVLHQDWLNGKEY | 3×(G4S) |
| | | KCKVSNKALPAPIEKTISKAKGQPREPRVYTLPPSRDELTKNQVSLTCLVKG | (SEQ |
| | | FYPSDIAVEWESNGQPENNYKTTPPVLVSDGSFTLYSKLTVDKSRWQQGNVF | ID NO: |
| | | SCSVMHEALHNHYTQKSLSLSPGGGGGSGGGGSGGGGSAVNGTSQFTCFYNS | 1142)- |
| | | RANISCVWSQDGALQDTSCQVHAWPDRRRWNQTCELLPVSQASWACNLILGA | IL2Rb (1- |
| | | PDSQKLTTVDIVTLRVLCREGVRWRVMAIQDFKPFENLRLMAPISLQVVHVE | 213) |
| | | THRCNISWEISQASHYFERHLEFEARTLSPGHTWEEAPLLTLKQKQEWICLE | |
| | | TLTPDTQYEFQVRVKPLQGEFTTWSPWSQPLAFRTKPAALGKD | |
| 1127 | Construct | EPKSSDKTHTCPPCPAPELLGGPSVFLFPPKPKDTLMISRTPEVTCVVVDVS | Fc knob- |
| | YYYY | HEDPEVKFNWYVDGVEVHNAKTKPREEQYNSTYRVVSVLTVLHQDWLNGKEY | MMPcs1-hu |
| | | KCKVSNKALPAPIEKTISKAKGQPREPQVYTLPPSRDELTENQVSLTCLVKG | IL15Ra (1- |
| | | FYPSDIAVEWESNGQPENNYKTTPPVLDSDGSFFLYSWLTVDKSRWQQGNVF | 77)-linker- |
| | | SCSVMHEALHNRFTQKSLSLSPGGGGSGGGGGPLGVRGGGGGSITCPPPMSV | hu IL15 |
| | | EHADIWVKSYSLYSRERYICNSGFKRKAGTSSLTECVLNKATNVAHWTTPSL | |
| | | KCIRDPALVHQRPAPPSGGSGGGGSGGGSGGGGSLQNWVNVISDLKKIEDLI | |
| | | QSMHIDATLYTESDVHPSCKVTAMKCFLLELQVISLESGDASIHDTVENLII | |
| | | LANNSLSSNGNVTESGCKECEELEEKNIKEFLQSFVHIVQMFINTS | |
| 1128 | Construct | ITCPPPMSVEHADIWVKSYSLYSRERYICNSGFKRKAGTSSLTECVLNKATN | hu IL 15Ra |
| | ZZZZ | VAHWTTPSLKCIRDPALVHQRPAPPSGGSGGGGSGGGSGGGGSLQNWVNVIS | (1-77)- |
| | | DLKKIEDLIQSMHIDATLYTESDVHPSCKVTAMKCFLLELQVISLESGDASI | linker-hu |
| | | HDTVENLIILANNSLSSNGNVTESGCKECEELEEKNIKEFLQSFVHIVQMFI | IL15-(SG3) |
| | | NTSSGGGGPLGVRGGGGGSGGGGSEPKSSDKTHTCPPCPAPELLGGPSVFLF | (SEQ ID |
| | | PPKPKDTLMISRTPEVTCVVVDVSHEDPEVKFNWYVDGVEVHNAKTKPREEQ | NO: 1158)- |
| | | YNSTYRVVSVLTVLHQDWLNGKEYKCKVSNKALPAPIEKTISKAKGQPREPQ | GPLGVRG |
| | | VYTLPPSRDELTENQVSLTCLVKGFYPSDIAVEWESNGQPENNYKTTPPVLD | (SEQ ID |
| | | SDGSFFLYSWLTVDKSRWQQGNVFSCSVMHEALHNRFTQKSLSLSPG | NO: 80)- |
| | | | 2×(G4S)(SEQ |
| | | | ID NO: |
| | | | 1142)-hIgG1 |
| | | | Fc knob |
| 1129 | Construct | EPKSSDKTHTCPPCPAPELLGGPSVFLFPPKPKDTLMISRTPEVTCVVVDVS | Fc knob- |
| | AAAAA | HEDPEVKFNWYVDGVEVHNAKTKPREEQYNSTYRVVSVLTVLHQDWLNGKEY | MMPcs1-hu |
| | | KCKVSNKALPAPIEKTISKAKGQPREPQVYTLPPSRDELTENQVSLTCLVKG | IL15Ra (1- |
| | | FYPSDIAVEWESNGQPENNYKTTPPVLDSDGSFFLYSWLTVDKSRWQQGNVF | 77)-linker- |
| | | SCSVMHEALHNRFTQKSLSLSPGGGGSGGGGGPLGVRGGGGGSITCPPPMSV | hu IL 15- |
| | | EHADIWVKSYSLYSRERYICNSGFKRKAGTSSLTECVLNKATNVAHWTTPSL | KLWVLPK |
| | | KCIRDPALVHQRPAPPSGGSGGGGSGGGSGGGGSLQNWVNVISDLKKIEDLI | (SEQ ID |
| | | QSMHIDATLYTESDVHPSCKVTAMKCFLLELQVISLESGDASIHDTVENLII | NO: 200) |
| | | LANNSLSSNGNVTESGCKECEELEEKNIKEFLQSFVHIVQMFINTSGSGGKL | |
| | | WVLPK | |
| 1130 | Construct | EPKSSDKTHTCPPCPAPELLGGPSVFLFPPKPKDTLMISRTPEVTCVVVDVS | Fc knob- |
| | BBBBB | HEDPEVKFNWYVDGVEVHNAKTKPREEQYNSTYRVVSVLTVLHQDWLNGKEY | MMPscr-hu |
| | | KCKVSNKALPAPIEKTISKAKGQPREPQVYTLPPSRDELTENQVSLTCLVKG | IL15Ra (1- |
| | | FYPSDIAVEWESNGQPENNYKTTPPVLDSDGSFFLYSWLTVDKSRWQQGNVF | 77)-linker- |
| | | SCSVMHEALHNRFTQKSLSLSPGGGGSGGGGGVRLGPGGGGGSITCPPPMSV | hu IL15- |
| | | EHADIWVKSYSLYSRERYICNSGFKRKAGTSSLTECVLNKATNVAHWTTPSL | KLWVLPK |
| | | KCIRDPALVHQRPAPPSGGSGGGGSGGGSGGGGSLQNWVNVISDLKKIEDLI | (SEQ ID |
| | | QSMHIDATLYTESDVHPSCKVTAMKCFLLELQVISLESGDASIHDTVENLII | NO: 200) |
| | | LANNSLSSNGNVTESGCKECEELEEKNIKEFLQSFVHIVQMFINTSGSGGKL | |
| | | WVLPK | |
| 1131 | Construct | EPKSSDKTHTCPPCPAPELLGGPSVFLFPPKPKDTLMISRTPEVTCVVVDVS | Fc knob- |
| | CCCCC | HEDPEVKFNWYVDGVEVHNAKTKPREEQYNSTYRVVSVLTVLHQDWLNGKEY | MMPcs1-hu |
| | | KCKVSNKALPAPIEKTISKAKGQPREPQVYTLPPSRDELTENQVSLTCLVKG | IL15Ra (1- |
| | | FYPSDIAVEWESNGQPENNYKTTPPVLDSDGSFFLYSWLTVDKSRWQQGNVF | 77)-linker- |
| | | SCSVMHEALHNRFTQKSLSLSPGGGGGGGGGPLGVRGGGGGSITCPPPMSV | hu IL15- |
| | | EHADIWVKSYSLYSRERYICNSGFKRKAGTSSLTECVLNKATNVAHWTTPSL | LRELHLDN |
| | | KCIRDPALVHQRPAPPSGGSGGGGSGGGSGGGGSLQNWVNVISDLKKIEDLI | N (SEQ ID |
| | | QSMHIDATLYTESDVHPSCKVTAMKCFLLELQVISLESGDASIHDTVENLII | NO: 188) |
| | | LANNSLSSNGNVTESGCKECEELEEKNIKEFLQSFVHIVQMFINTSGSGGLR | |
| | | ELHLDNN | |
| 1132 | Construct | EPKSSDKTHTCPPCPAPELLGGPSVFLFPPKPKDTLMISRTPEVTCVVVDVS | Fc knob- |
| | DDDDD | HEDPEVKFNWYVDGVEVHNAKTKPREEQYNSTYRVVSVLTVLHQDWLNGKEY | MMPscr-hu |
| | | KCKVSNKALPAPIEKTISKAKGQPREPQVYTLPPSRDELTENQVSLTCLVKG | IL15Ra (1- |
| | | FYPSDIAVEWESNGQPENNYKTTPPVLDSDGSFFLYSWLTVDKSRWQQGNVF | 77)-linker- |
| | | SCSVMHEALHNRFTQKSLSLSPGGGGSGGGGGVRLGPGGGGGSITCPPPMSV | hu IL15- |
| | | EHADIWVKSYSLYSRERYICNSGFKRKAGTSSLTECVLNKATNVAHWTTPSL | LRELHLDN |
| | | KCIRDPALVHQRPAPPSGGSGGGGSGGGSGGGGSLQNWVNVISDLKKIEDLI | N (SEQ ID |
| | | QSMHIDATLYTESDVHPSCKVTAMKCFLLELQVISLESGDASIHDTVENLII | NO: 188) |
| | | LANNSLSSNGNVTESGCKECEELEEKNIKEFLQSFVHIVQMFINTSGSGGLR | |
| | | ELHLDNN | |
| 1133 | Construct | ITCPPPMSVEHADIWVKSYSLYSRERYICNSGFKRKAGTSSLTECVLNKATN | hu IL15Ra |
| | EEEEE | VAHWTTPSLKCIRDPALVHQRPAPPSGGSGGGGSGGGSGGGGSLQNWVNVIS | (1-77)- |
| | | DLKKIEDLIQSMHIDATLYTESDVHPSCKVTAMKCFLLELQVISLESGDASI | linker-hu |
| | | HDTVENLIILANNSLSSNGNVTESGCKECEELEEKNIKEFLQSFVHIVQMFI | IL15- |
| | | NTSGGGKLWVLPKGGGGPLGVRGGGGGSGGGGSEPKSSDKTHTCPPCPAPEL | KLWVLPK |
| | | LGGPSVFLFPPKPKDTLMISRTPEVTCVVVDVSHEDPEVKFNWYVDGVEVHN | (SEQ ID |
| | | AKTKPREEQYNSTYRVVSVLTVLHQDWLNGKEYKCKVSNKALPAPIEKTISK | NO: 200)- |
| | | AKGQPREPQVYTLPPSRDELTENQVSLTCLVKGFYPSDIAVEWESNGQPENN | MMPcs1-Fc |
| | | YKTTPPVLDSDGSFFLYSWLTVDKSRWQQGNVFSCSVMHEALHNRFTQKSLS | knob |
| | | LSPG | |
| 1134 | Construct | ITCPPPMSVEHADIWVKSYSLYSRERYICNSGFKRKAGTSSLTECVLNKATN | hu IL 15Ra |
| | FFFFF | VAHWTTPSLKCIRDPALVHQRPAPPSGGSGGGGSGGGSGGGGSLQNWVNVIS | (1-77)- |
| | | DLKKIEDLIQSMHIDATLYTESDVHPSCKVTAMKCFLLELQVISLESGDASI | linker-hu |
| | | HDTVENLIILANNSLSSNGNVTESGCKECEELEEKNIKEFLQSFVHIVQMFI | IL15- |
| | | NTSGGGKLWVLPKGGGGVRLGPGGGGGSGGGGSEPKSSDKTHTCPPCPAPEL | KLWVLPK |
| | | LGGPSVFLFPPKPKDTLMISRTPEVTCVVVDVSHEDPEVKFNWYVDGVEVHN | (SEQ ID |
| | | AKTKPREEQYNSTYRVVSVLTVLHQDWLNGKEYKCKVSNKALPAPIEKTISK | NO: 200)- |
| | | AKGQPREPQVYTLPPSRDELTENQVSLTCLVKGFYPSDIAVEWESNGQPENN | MMPscr-Fc |
| | | YKTTPPVLDSDGSFFLYSWLTVDKSRWQQGNVFSCSVMHEALHNRFTQKSLS | knob |
| | | LSPG | |
| 1135 | Construct | ITCPPPMSVEHADIWVKSYSLYSRERYICNSGFKRKAGTSSLTECVLNKATN | hu IL 15Ra |
| | GGGGG | VAHWTTPSLKCIRDPALVHQRPAPPSGGSGGGGSGGGSGGGGSLQNWVNVIS | (1-77)- |
| | | DLKKIEDLIQSMHIDATLYTESDVHPSCKVTAMKCFLLELQVISLESGDASI | linker-hu |
| | | HDTVENLIILANNSLSSNGNVTESGCKECEELEEKNIKEFLQSFVHIVQMFI | IL15- |
| | | NTSGGGLRELHLDNNGGGGPLGVRGGGGGSGGGGSEPKSSDKTHTCPPCPAP | LRELHLDN |
| | | ELLGGPSVFLFPPKPKDTLMISRTPEVTCVVVDVSHEDPEVKFNWYVDGVEV | N (SEQ ID |
| | | HNAKTKPREEQYNSTYRVVSVLTVLHQDWLNGKEYKCKVSNKALPAPIEKTI | NO: 188)- |
| | | SKAKGQPREPQVYTLPPSRDELTENQVSLTCLVKGFYPSDIAVEWESNGQPE | MMPcs1-Fc |
| | | NNYKTTPPVLDSDGSFFLYSWLTVDKSRWQQGNVFSCSVMHEALHNRFTQKS | knob |
| | | LSLSPG | |
| 1136 | Construct | ITCPPPMSVEHADIWVKSYSLYSRERYICNSGFKRKAGTSSLTECVLNKATN | hu IL 15Ra |
| | HHHHH | VAHWTTPSLKCIRDPALVHQRPAPPSGGSGGGGSGGGSGGGGSLQNWVNVIS | (1-77)- |
| | | DLKKIEDLIQSMHIDATLYTESDVHPSCKVTAMKCFLLELQVISLESGDASI | linker-hu |
| | | HDTVENLIILANNSLSSNGNVTESGCKECEELEEKNIKEFLQSFVHIVQMFI | IL15- |
| | | NTSGGGLRELHLDNNGGGGPLGVRGGGGGSGGGGSEPKSSDKTHTCPPCPAP | LRELHLDN |
| | | ELLGGPSVFLFPPKPKDTLMISRTPEVTCVVVDVSHEDPEVKFNWYVDGVEV | N (SEQ ID |
| | | HNAKTKPREEQYNSTYRVVSVLTVLHQDWLNGKEYKCKVSNKALPAPIEKTI | NO: 188)- |
| | | SKAKGQPREPQVYTLPPSRDELTENQVSLTCLVKGFYPSDIAVEWESNGQPE | MMPscr-Fc |
| | | NNYKTTPPVLDSDGSFFLYSWLTVDKSRWQQGNVFSCSVMHEALHNRFTQKS | knob |
| | | LSLSPG | |
| 1137 | Construct | ITCPPPMSVEHADIWVKSYSLYSRERYICNSGFKRKAGTSSLTECVLNKATN | hu IL15Ra |
| | PPPP | VAHWTTPSLKCIRDPALVHQRPAPPSGGSGGGGSGGGSGGGGSLQNWVNVIS | (1-77)- |
| | | DLKKIEDLIQSMHIDATLYTESDVHPSCKVTAMKCFLLELQVISLESGDASI | linker-hu |
| | | HDTVENLIILANNSLSSNGNVTESGCKECEELEEKNIKEFLQSFVHIVQMFI | IL15-(SG3) |
| | | NTSSGGGGPLGVRGGGGGSGGGGSGGGGSGGGGSAVNGTSQFTCFYNSRANI | (SEQ ID |
| | | SCVWSQDGALQDTSCQVHAWPDRRRWNQTCELLPVSQASWACNLILGAPDSQ | NO: 1158)- |
| | | KLTTVDIVTLRVLCREGVRWRVMAIQDFKPFENLRLMAPISLQVVHVETHRC | GPLGVRG |
| | | NISWEISQASHYFERHLEFEARTLSPGHTWEEAPLLTLKQKQEWICLETLTP | (SEQ ID |
| | | DTQYEFQVRVKPLQGEFTTWSPWSQPLAFRTKPAALGKDTGGGGSGEPKSSD | NO: 80)- |
| | | KTHTCPPCPAPEAAGGPSVFLFPPKPKDTLMISRTPEVTCVVVDVSHEDPEV | 4×(G4S)(SEQ |
| | | KFNWYVDGVEVHNAKTKPREEQYNSTYRVVSVLTVLHQDWLNGKEYKCKVSN | ID NO: |
| | | KALPAPIEKTISKAKGQPREPQVYTLPPSRDELTKNQVSLTCLVKGFYPSDI | 1142)-IL2Rb |
| | | AVEWESNGQPENNYKTTPPVLDSDGSFFLYSKLTVDKSRWQQGNVFSCSVMH | (1-214)- |
| | | EALHNHYTQKSLSLSPG | (G4SG) |
| | | | (SEQ ID |
| | | | NO: 1162)- |
| | | | Hu IgG1 Fc |

TABLE 2
Table of Targeting Sequences

| SEQ ID | | | | |
|---|---|---|---|---|
| NO | Sequence | Binds to | Note 1 | Note 2 |
| 179 | (TLTYTWS)n | denatured collagen | binding to MMP degraded collagen | |
| | | IV | | |
| 180 | (CREKA)n | denatured collagen | binding to MMP degraded | inhibit tumor vasculature |
| | | IV | collagen | formation |
| 181 | (GXY)n | denatured Collagen | Gly = Glycine/X = Proline or | This peptide binds to |
| | | | modified Proline/Y = Proline | collagen preteolytically |
| | | | or modified Proline | digested by MMP |
| 182 | GHCVTDSGVVYSVGM | denatured Collagen | from Fibronectin Domain 1-6 | |
| | QWLKTQGNKQMLCTC | | | |
| | LGNGVSCQET | | | |
| 183 | EICTTNEGVMYRIGDQ | denatured Collagen | from Fibronectin Domain 1-7 | |
| | WDKQHDMGHMMRCT | | | |
| | CVGNGRGEWTCIAY | | | |
| 184 | DQCIVDDITYNVNDTFH | denatured Collagen | from Fibronectin Domain 1-8 | |
| | KRHEEGHMLNCTCFGQ | | | |
| | GRGRWKCDPV | | | |
| 185 | DQCQDSETGTFYQIGDS | denatured Collagen | from Fibronectin Domain 1-9 | |
| | WEKYVHGVRYQCYCY | | | |
| | GRGIGEWHCQPL | | | |
| 186 | SNGEPCVLPFTYNGRTF | denatured Collagen | from Fibronectin Domain 2-1 | |
| | YSCTTEGRQDGHLWCS | | | |
| | TTSNYEQDQKYSFCTD | | | |
| 187 | SNGALCHFPFLYNNHN | denatured Collagen | from Fibronectin Domain 2-2 | |
| | YTDCTSEGRRDNMKW | | | |
| | CGTTQNYDADQKFGFC | | | |
| | PM | | | |
| 188 | LRELHLDNN | Collagen type I | | |
| 189 | RRANAALKAGELYKSI | Collagen type I | Kd 0.86 uM // 860 nM | Differential binding |
| | LYGC | | | affinity to Collagen |
| 190 | RRANAALKAGELYKCI | Collagen type I | Kd: 10 nM (tight binding) | Differential binding |
| | LYGC | | | affinity to Collagen |
| 191 | MIVIELGTNPLKSSGIEN | Collagen type I | Kd 0.394 uM // 394 nM | Differential binding |
| | GAFQGMKK | | | affinity to Collagen |
| 192 | LRELHLNNN | Collagen type I | Kd 0.17 uM //170 nM | Differential binding |
| | | | | affinity to Collagen |
| 193 | WREPSFCALS | Collagen type I | Kd 100 uM // 100,000 nM | Differential binding |
| | | | | affinity to Collagen |
| 194 | TKKTLRT | Collagen type I | Kd ≤100 uM | Differential binding |
| | | | | affinity to Collagen |
| 195 | CPKESCNLFVLKD | Collagen type I | Kd 0.681 uM //681 nM | Differential binding |
| | | | | affinity to Collagen |
| 196 | WREPSFCALS | Collagen type I | Kd : 100 uM // 100,000 nM | Differential binding |
| | | | | Collagen |
| 197 | HVWMQAPGGGK | Collagen type I | Kd 61 uM // 61,000 nM | H-V-F/W-Q/M-Q-P/A-P/K |
| | | | | motif |
| 198 | HVWMQAPGGGC | Collagen type I | | |
| 199 | WYRGRL | Collagen type II | | |
| 200 | KLWVLPK | Collagen type IV | | |
| 201 | RRANAALKAGELYKSI | Collagen | | |
| | LY | | | |
| 202 | GELYKSILY | Collagen | | |
| 203 | RRANAALKAGELYKCI | Collagen | | |
| | LY | | | |
| 204 | GELYKCILY | Collagen | | |
| 205 | RLDGNEIKR | Collagen | | |
| 206 | AHEEISTTNEGVM | Collagen | | |
| 207 | NGVFKYRPRYFLYKHA | Collagen | | |
| | YFYPPLKRFPVQ | | | |
| 208 | CQDSETRTFY | Collagen | | |
| 209 | TKKTLRT | Collagen | | |
| 210 | GLRSKSKKFRRPDIQYP | Collagen | | |
| | DATDEDITSHM | | | |
| 211 | SQNPVQP | Collagen | | |
| 212 | SYIRIADTNIT | Collagen | | |
| 213 | KELNLVYT | Collagen | | |
| 214 | GSIT | Collagen | | |
| 215 | GSITTIDVPWNV | Collagen | | |
| 216 | GQLYKSILY | Collagen | | |
| 217 | RRANAALKAGQLYKSI | Collagen | | |
| | LY | | | |
| 218 | WREPSFCALS | Collagen | | |
| 219 | WHCTTKFPHHYCLY | Collagen | | |
| 220 | AHKCPWHLYTTHYCFT | Collagen | | |
| 221 | PAHKCPWHLYTHYCFT | Collagen | | |
| 222 | GROGER | Collagen | O is 4-hydroxyproline (see, Raynal, N., et al., J. Biol. | |
| | | | Chem., 2006, 281(7), 3821-3831) | |
| 223 | GMOGER | Collagen | O is 4-hydroxyproline (see, Raynal, N., et al., J. Biol. | |
| | | | Chem., 2006, 281(7), 3821-3831) | |
| 224 | GLOGEN | Collagen | O is 4-hydroxyproline (see, Raynal, N., et al., J. Biol. | |
| | | | Chem., 2006, 281(7), 3821-3831) | |
| 225 | GLOGER | Collagen | O is 4-hydroxyproline (see, Raynal, N., et al., J. Biol. | |
| | | | Chem., 2006, 281(7), 3821-3831) | |
| 226 | GLKGEN | Collagen | O is 4-hydroxyproline (see, Raynal, N., et al., J. Biol. | |
| | | | Chem., 2006, 281(7), 3821-3831) | |
| 227 | GFOGERGVEGPOGPA | Collagen | O is 4-hydroxyproline (see, Raynal, N., et al., J. Biol. | |
| | | | Chem., 2006, 281(7), 3821-3831) | |
| 228 | WREPSFCALS | Collagen | | Takagi, J., et al, |
| | | | | Biochemistry, |
| | | | | 1992, 31, 8530-8534 |
| 229 | WYRGRL | Collagen | | Rothenfluh D.A., et al, |
| | | | | Nat Mater. 2008, |
| | | | | 7(3), 748-54 |
| 230 | WTCSGDEYTWHC | Collagen | | |
| 231 | WTCVGDHKTWKC | Collagen | | |
| 232 | QWHCTTRFPHHYCLYG | Collagen | | U.S. 2007/0293656) |
| 233 | STWTWNGSAWTWNEG | Collagen | | |
| | GK | | | |
| 234 | STWTWNGTNWTRNDG | Collagen | | WO/2014/059530 |
| | GK | | | |
| 235 | CVWLWEQC | Collagen | | |
| 236 | CMTSPWRC | Collagen | | Vanhoorelbeke, K., et al, |
| | | | | J. Biol. Chem., 2003, |
| | | | | 278, 37815-37821 |
| 237 | CPGRVMHGLHLGDDE | Collagen | | Muzzard, J., et al, PLOS |
| | GPC | | | one. 4 (e5585) I- 10) |
| 238 | KLWLLPK | Collagen | | Chan, J. M., et al, Proc |
| | | | | Natl Acad Sci |
| | | | | U.S.A., 2010, 107, |
| | | | | 2213- 2218) |
| 239 | CQDSETRTFY | Collagen | | U.S. 2013/0243700 |
| 240 | LSELRLHEN | Collagen | | Fredrico, S., Angew. |
| | | | | Chem. Int. Ed. 2015, |
| | | | | 37, 10980-10984 |
| 241 | LTELHLDNN | Collagen | | Fredrico, S., Angew. |
| | | | | Chem. Int. Ed. 2015, |
| | | | | 37, 10980-10985 |
| 242 | LSELRLHNN | Collagen | | Fredrico, S., Angew. |
| | | | | Chem. Int. Ed. 2015, |
| | | | | 37, 10980-10986 |
| 243 | LSELRLHAN | Collagen | | Fredrico, S., Angew. |
| | | | | Chem. Int. Ed. 2015, |
| | | | | 37, 10980-10987 |
| 244 | LRELHLNNN | Collagen | | Fredrico, S., Angew. |
| | | | | Chem. Int. Ed. 2015, |
| | | | | 37, 10980-10988 |
| 245 | RVMHGLHLGDDE | Collagen | | |
| 246 | RVMHGLHLGNNQ | Collagen | | |
| 247 | RVMHGLHLGNNQ | Collagen | | |
| 248 | GQLYKSILYGSG-4K2K | Collagen | (a 4-branch peptide) which can be conjugated to a | |
| | | | fusion polypeptide | |
| 249 | GSGQLYKSILY | Collagen | | |
| 250 | GSGGQLYKSILY | Collagen | | |
| 251 | KQLNLVYT | Collagen | | |
| 252 | CVWLWQQC | Collagen | | |
| 253 | WREPSFSALS | Collagen | | |
| 254 | GHRPLDKKREEAPSLRP | Collagen | | |
| | APPPISGGGYR | | | |
| 255 | GHRPLNKKRQQ | Collagen | | |
| | APSLRPAPPPISGGGYR | | | |
| 256 | GELYKSILYGSG | Collagen | | |
| 257 | GQLYKSILYGSG | Collagen | | |
| 258 | RYPISRPRKRGSG | Collagen | | |
| 259 | GELYKSILYGC | Collagen | | |
| 260 | RLDGNEIKRGC | Collagen | | |
| 261 | AHEEISTTNEGVMGC | Collagen | | |
| 262 | GCGGELYKSILY | Collagen | | |
| 263 | NGVFKYRPRYFLYKHA | Collagen | | |
| | YFYPPLKRFPVQGC | | | |
| 264 | CQDSETRTFYGC | Collagen | | |
| 265 | TKKTLRTGC | Collagen | | |
| 266 | GLRSKSKKFRRPDIQYP | Collagen | | |
| | DATDEDITSHMGC | | | |
| 267 | SQNPVQPGC | Collagen | | |
| 268 | SYIRIADTNITGC | Collagen | | |
| 269 | KELNLVYTGC | Collagen | | |
| 270 | GSITTIDVPWNVGC | Collagen | | |
| 271 | GCGGELYKSILYGC | Collagen | | |
| 272 | RRANAALKAGELYKSI | Collagen | | |
| | LYGSG | | | |
| 273 | cyclic CVWLWENC | Collagen | cyclic peptides can be conjugated to a fusion | |
| | | | polypeptide | |
| 274 | cyclic CVWLWEQC | Collagen | cyclic peptides can be | Depraetere H., et al, |
| | | | conjugated to a | Blood. 1998, 92, |
| | | | fusion polypeptide | 4207-421 1; |
| | | | | and Duncan R., |
| | | | | Nat Rev Drug Discov, |
| | | | | 2003, 2(5), 347-360 |
| 275 | D-amino acid | Collagen | D-amino acid-containing peptides can be conjugated to | |
| | EDDGLHLGHMVR | | linker polypeptide | |
| 276 | D-amino acid | Collagen | D-amino acid-containing peptides can be conjugated to | |
| | QNNGLHLGHMVR | | linker polypeptide | |
| 277 | PPTDLRFTNIGPDTMRV | integrin | from Fibronectin Domain III-9 | |
| | TWAPPPSIDLTNFLVRY | | | |
| | SPVKNEEDVAELSISPS | | | |
| | DNAVVLTNLLPGTEYV | | | |
| | VSVSSVYEQHESTPLRG | | | |
| | RQKTGLDSP | | | |
| 278 | TGIDFSDITANSFTVHWI | integrin | from Fibronectin Domain III-10 | |
| | APRATITGYRIRHHPEH | | | |
| | FSGRPREDRVPHSRNSI | | | |
| | TLTNLTPGTEYVVSIVA | | | |
| | LNGREESPLLIGQQSTV | | | |
| | SD | | | |
| 279 | PGCYDNGKHYQINQQ | integrin | from Fibronectin Domain 1-1 | |
| | WERTYLGNALVCTCYG | | | |
| | GSRGENCESK | | | |
| 280 | ETCFDKYTGNTYRVGD | integrin | from Fibronectin Domain 1-2 | |
| | TYERPKDSMIWDCTCIG | | | |
| | AGRGRISCTIA | | | |
| 281 | NRCHEGGQSYKIGDTW | integrin | from Fibronectin Domain 1-3 | |
| | RRPHETGGYMLECVCL | | | |
| | GNGKGEWTCKPI | | | |
| 282 | EKCFDHAAGTSYVVGE | integrin | from Fibronectin Domain 1-4 | |
| | TWEKPYQGWMMVDCT | | | |
| | CLGEGSGRITCTSR | | | |
| 283 | NRCNDQDTRTSYRIGD | integrin | from Fibronectin Domain 1-5 | |
| | TWSKKDNRGNLLQCIC | | | |
| | TGNGRGEWKCERH | | | |
| 284 | GHCVTDSGVVYSVGM | denatured Collagen/ | from Fibronectin Domain 1-6 | duplicated in collagen |
| | QWLKTQGNKQMLCTC | integrin | | |
| | LGNGVSCQET | | | |
| 285 | EICTTNEGVMYRIGDQ | denatured Collagen/ | from Fibronectin Domain 1-7 | duplicated in collagen |
| | WDKQHDMGHMMRCT | integrin | | |
| | CVGNGRGEWTCIAY | | | |
| 286 | DQCIVDDITYNVNDTFH | denatured Collagen/ | from Fibronectin Domain 1-8 | duplicated in collagen |
| | KRHEEGHMLNCTCFGQ | integrin | | |
| | GRGRWKCDPV | | | |
| 287 | DQCQDSETGTFYQIGDS | denatured Collagen/ | from Fibronectin Domain 1-9 | duplicated in collagen |
| | WEKYVHGVRYQCYCY | integrin | | |
| | GRGIGEWHCQPL | | | |
| 288 | APTDLKFTQVTPTSLSA | integrin | from Fibronectin Domain III-14 | |
| | QWTPPNVQLTGYRVRV | | | |
| | TPKEKTGPMKEINLAPD | | | |
| | SSSVVVSGLMVATKYE | | | |
| | VSVYALKDTLTSRPAQ | | | |
| | GVVTTLENVSPP | | | |
| 289 | APTNLQFVNETDSTVL | integrin | from Fibronectin Domain III-5 | |
| | VRWTPPRAQITGYRLT | | | |
| | VGLTRRGQPRQYNVGP | | | |
| | SVSKYPLRNLQPASEYT | | | |
| | VSLVAIKGNQESPKATG | | | |
| | VFTTLQPG | | | |
| 290 | KGHRGF | integrin | Derived from Collagen I | |
| 291 | GFPGER | integrin | Derived from Collagen I | |
| 292 | GTPGPQGIAGQRDVV | integrin | Derived from Collagen alpha1(I) | |
| 293 | EKGPD | integrin | Derived from Collagen II | |
| 294 | EKGPDP | integrin | Derived from Collagen II | |
| 295 | EKGPDPL | integrin | Derived from Collagen II | |
| 296 | TAGSCLRKFSTM | integrin | Derived from Collagen IV | |
| 297 | TAIPSCPEGTVPLYS | integrin | Derived from Collagen alpha3(IV)-NC1 | |
| 298 | TDIPPCPHGWISLWK | integrin | Derived from Collagen IV | |
| 299 | PHSRN | integrin | Derived from Fibronectin | |
| 300 | RGD | integrin | Derived from Fibronectin | |
| 301 | GRGDSP | integrin | Derived from Fibronectin | |
| 302 | YRVRVTPKEKTGPMKE | integrin | Derived from Fibronectin | |
| 303 | SPPRRARVT | integrin | Derived from Fibronectin | |
| 304 | WQPPRARI | integrin | Derived from Fibronectin | |
| 305 | KNNQKSEPLIGRKKT | integrin | Derived from Fibronectin | |
| 306 | EILDVPST | integrin | Derived from Fibronectin | |
| 307 | REDV | integrin | Derived from Fibronectin | |
| 308 | RQVFQVAYIIIKA | integrin | Derived from Laminin Alpha-1 chain | |
| 309 | SINNTAVMQRLT | integrin | Derived from Laminin Alpha-1 chain | |
| 310 | IKVAV | integrin | Derived from Laminin Alpha-1 chain | |
| 311 | NRWHSIYITRFG | integrin | Derived from Laminin Alpha-1 chain | |
| 312 | TWYKIAFQRNRK | integrin | Derived from Laminin Alpha-1 chain | |
| 313 | RKRLQVQLSIRT | integrin | Derived from Laminin Alpha-1 chain | |
| 314 | KNRLTIELEVRT | integrin | Derived from Laminin Alpha-2 chain | |
| 315 | SYWYRIEASRTG | integrin | Derived from Laminin Alpha-2 chain | |
| 316 | DFGTVQLRNGFPFFSYD | integrin | Derived from Laminin Alpha-2 chain | |
| | LG | | | |
| 317 | GQLFHVAYILIKF | integrin | Derived from Laminin Alpha-3 chain | |
| 318 | KNSFMALYLSKG | integrin | Derived from Laminin Alpha-3 chain | |
| 319 | TLFLAHGRLVFM | integrin | Derived from Laminin Alpha-4 chain | |
| 320 | GQVFHVAYVLIKF | integrin | Derived from Laminin Alpha-5 chain | |
| 321 | GIIFFL | integrin | Derived from Laminin Alpha-5 chain | |
| 322 | LALFLSNGHFVA | integrin | Derived from Laminin Alpha-5 chain | |
| 323 | RYVVLPR | integrin | Derived from Laminin Beta-1 chain | |
| 324 | PDSGR | integrin | Derived from Laminin Beta-1 chain | |
| 325 | YIGSR | integrin | Derived from Laminin Beta-1 chain | |
| 326 | KAFDITYVRLKF | integrin | Derived from Laminin Gamma-1 chain | |
| 327 | RNIAEIIKDI | integrin | Derived from Laminin Gamma-1 chain | |
| 328 | FRHRNRKGY | integrin | Derived from Vitronectin | |
| 329 | KKQRFRHRNRKGYRSQ | integrin | Derived from Vitronectin | |
| 330 | FHRRIKA | integrin | Derived from Sialoprotein | |
| 331 | KRSR | integrin | Derived from Sialoprotein | |
| 332 | GLPGER | α1β1, α2β1 | Derived from Collagen α1(1) 7S | |
| 333 | GFPGER | α1β1, α2β1 | Derived from Collagen alpha1(I) | |
| 334 | GLSGER | α2β1 | Derived from Collagen alphal(I) | |
| 335 | DGEA | α2β1 | Derived from Collagen alpha1(I) | |
| 336 | GPAGKDGEAGAQG | α2β1 | Derived from Collagen alpha1(I) | |
| 337 | GPKGAAGEPGKP | α1β1, α3β1 | Derived from Collagen alpha1(I) | |
| 338 | GAPGPKGARGSA | α1β1, α3β1 | Derived from Collagen alpha1(I) | |
| 339 | GPQGIAGQRGVVGLP | α1β1 | Derived from Collagen alpha1(I) | |
| 340 | PKGQKGEKG | Poly(I) | Derived from Collagen alpha1(I) | |
| 341 | GASGER | α2β1 | Derived from Collagen alpha1(I) | |
| 342 | GQRGER | α2β1 | Derived from Collagen alpha1(I) | |
| 343 | GMPGER | integrin | Derived from Collagen alpha1(I) | |
| 344 | RGQPGVMGF | VWF | Derived from Collagen alpha1(III) | |
| 345 | GKDGES | α2β1 | Derived from Collagen alpha1(III) | |
| 346 | GLKGEN | α2β1 | Derived from Collagen alpha1(III) | |
| 347 | GLPGEN | α2β1 | Derived from Collagen alpha1(III) | |
| 348 | GLPGEA | α2β1 | Derived from Collagen alpha1(III) | |
| 349 | GPPGDQGPPGIP | α1β1 | Derived from Collagen alpha1(IV) | |
| 350 | GAKGRAGFPGLP | α1β1 | Derived from Collagen alpha1(IV) | |
| 351 | MFKKPTPSTLKAGELR | integrin | Derived from Collagen alpha1(IV) | |
| 352 | GFPGSRGDTGPP | integrin | Derived from Collagen alpha1(IV) | |
| 353 | GVKGDKGNPGWPGAP | integrin | Derived from Collagen alpha1(IV) | |
| 354 | FYFDLR | α1β1, α2β1 | Derived from Collagen alpha1(IV) | |
| 355 | MFKKPTPSTLKAGELR | integrin | Derived from Collagen alpha1(IV) | |
| 356 | GFPGSRGDTGPP | integrin | Derived from Collagen alpha1(IV) | |
| 357 | GVKGDKGNPGWPGAP | integrin | Derived from Collagen alpha1(IV) | |
| 358 | FYFDLR | α1β1, α2β1 | Derived from Collagen alpha1(IV) | |
| 359 | RGQPGVPGVPGMKGD | integrin | Derived from Collagen alpha2(IV) | |
| 360 | TDIPPCPHGWISLWK | integrin | Derived from Collagen alpha3(IV)-NC1 | |
| 361 | MNYYSNS | integrin | Derived from Collagen alpha3(IV)-NC1 | |
| 362 | CNYYSNSYSFWLASLN | integrin | Derived from Collagen alpha3(IV)-NC1 | |
| | PER | | | |
| 363 | ISRCQVCMKKRH | integrin | Derived from Collagen alpha3(IV)-NC1 | |
| 364 | TLGSCLQRFTTM | integrin | Derived from Collagen alpha3(IV)-NC1 | |
| 365 | GRRGKT | integrin | Derived from Collagen alpha3(IV)-NC1 | |
| 366 | RGQPGRKGL | integrin | Derived from Collagen alpha3(IV)-NC1 | |
| 367 | MFRKPIPSTVKA | integrin | Derived from Collagen alpha3(IV)-NC1 | |
| 368 | IISRCQVCMKMRP | integrin | Derived from Collagen alpha3(IV)-NC1 | |
| 369 | LAGSCLPVESTL | integrin | Derived from Collagen alpha4(IV)-NC1 | |
| 370 | TAGSCLRRESTM | integrin | Derived from Collagen alpha5(IV)-NC1 | |
| 371 | NKRAHG | integrin | Derived from Collagen alpha5(IV)-NC2 | |
| 372 | WTPPRAQITGYRLTVG | α5β1 | Derived from Fibronectin III-5 | |
| | LTRR | | | |
| 373 | KLDAPT | α4β1, α4β7 | Derived from Fibronectin III-5 | |
| 374 | PHSRN | α5β1 | Derived from Fibronectin III-9 | |
| 375 | RGD | α5β1, αvβ3 | Derived from Fibronectin III-10 | |
| 376 | RGDS | αIIbβ3 | Derived from Fibronectin III-10 | |
| 377 | GRGDSP | α5β1 | Derived from Fibronectin III-10 | |
| 378 | EDGIHEL | α4β1, α9β1 | Derived from Fibronectin EDA | |
| 379 | PRARITGYIIKYEKPGSP | integrin | Derived from Fibronectin III-14 | |
| | PREVVPRPRPGV | | | |
| 380 | IDAPS | α4β1 | Derived from Fibronectin IIICS-1 | |
| 381 | VVIDASTAIDAPSNL | α4β1 | Derived from Fibronectin IIICS-1 | |
| 382 | LDVPS | α4β1 | Derived from Fibronectin IIICS-1 | |
| 383 | REDV | α4β1 | Derived from Fibronectin IIICS-5 | |
| 384 | PHSRN-RGDSP | α5β1 | Derived from Fibronectin III-10 | |
| 385 | PLDREAIAKY | integrin | Derived from E-Cadherin EC1 | |
| 386 | HAVDI | integrin | Derived from E-Cadherin EC1, groove | |
| 387 | LFSHAVSSNG | integrin | Derived from E-Cadherin EC1, groove | |
| 388 | ADTPPV | integrin | Derived from E-Cadherin EC1, bulge | |
| 389 | QGADTPPVGV | integrin | Derived from E-Cadherin EC1, bulge | |
| 390 | PLDREAIAKY | integrin | Derived from E-Cadherin EC1 | |
| 391 | DQNDN | integrin | Derived from E-Cadherin EC1 | |
| 392 | HAVDI | integrin | Derived from E-Cadherin EC1 | |
| 393 | LRAHAVDING | integrin | Derived from E-Cadherin EC1 | |
| 394 | LRAHAVDVNG | integrin | Derived from E-Cadherin EC1 | |
| 395 | VITVKDINDN | integrin | Derived from E-Cadherin EC2 | |
| 396 | GLDRESYPYY | integrin | Derived from E-Cadherin EC2 | |
| 397 | MKVSATDADD | integrin | Derived from E-Cadherin EC2 | |
| 398 | QDPELPDKNM | integrin | Derived from E-Cadherin EC2, bulge | |
| 399 | LVVQAADLQG | integrin | Derived from E-Cadherin EC2, groove | |
| 400 | NDDGGQFVVT | integrin | Derived from E-Cadherin EC3, bulge | |
| 401 | LVVQAADLQG | integrin | Derived from E-Cadherin EC2, groove | |
| 402 | TYRIWRDTAN | integrin | Derived from E-Cadherin EC4, bulge | |
| 403 | YILHVAVTNY | integrin | Derived from E-Cadherin EC3, groove | |
| 404 | YTALIIATDN | integrin | Derived from E-Cadherin EC4, groove | |
| 405 | QDPELPDKNM | integrin | Derived from E-Cadherin EC2, bulge | |
| 406 | RGDV | αvβ3, αvβ5 | Somatomedin B | |
| 407 | PQVTRGDVFTMP | αvβ3, αvβ5 | Somatomedin B | |
| 408 | LNRQELFPFG | integrin | Nidogen G2 | |
| 409 | SIGFRGDGQTC | integrin | Nidogen G2 | |
| 410 | TWSKVGGHLRPGIVQS | IgB | Perlecan IV | |
| | G | | | |
| 411 | VAEIDGIEL | α9β1 | Tenascin-C | |
| 412 | VFDNFVLK | α7β1 | Tenascin-C | |
| 413 | VGVAPG | integrin | Elastin | |
| 414 | PGVGV | integrin | Elastin | |
| 415 | TTSWSQCSKS | α6β1 | CCN-1 | |
| 416 | SVVYGLR | α9β1 | Osteopontin | |
| 417 | DGRGDSVAYG | ανβ3 | Osteopontin | |
| 418 | LALERKDHSG | α6β1 | Thrombospondin | |
| 419 | RGDF | αIIIbβ3 | Fibrinogen | |
| 420 | KRLDGSV | αMβ2 | Fibrinogen | |
| 421 | HHLGGAKQAGDV | αIIbβ3 | Fibrinogen | |
| 422 | YSMKKTTMKIIPFNRLT | αllbβ3 | Fibrinogen | |
| | IG | | | |
| 423 | GVYYQGGTYSKAS | αMB2 | Fibrinogen | |
| 424 | LWVTVRSQQRGLF | α5β1 | Laminin α1 LN (A3) | |
| 425 | GTNNWWQSPSIQN | α4β1, α4β7 | Laminin α1 LN (A10) | |
| 426 | WVTVTLDLRQVFQ | α5β1 | Laminin α1 LN (A12) | |
| 427 | RQVFQVAYIIIKA | α1β1, α2β1 | Laminin α1 LN (A13) | |
| 428 | LTRYKITPRRGPPT | α5β1 | Laminin α1 LN (A18) | |
| 429 | LLEFTSARYIRL | integrin | Laminin Laminin α1 LN (A24) | |
| 430 | YIRLRLORIRTL | integrin | Laminin α1 LN (A25) | |
| 431 | RRYYYSIKDISV | integrin | Laminin α1 V? (A29) | |
| 432 | GGFLKYTVSYDI | integrin | Laminin α1 L4a (A55) | |
| 433 | RDQLMTVLANVT | integrin | Laminin α1 L4a (A64) | |
| 434 | VLIKGGRARKHV | α5β1 | Laminin α1 L4a (A112) | |
| 435 | NLLLLLVKANLK | integrin | Laminin α1 L1 (A167) | |
| 436 | HRDELLLWARKI | integrin | Laminin α1 L1 (A174) | |
| 437 | KRRARDLVHRAE | integrin | Laminin α1 L1 (A177) | |
| 438 | SQFQESVDNITK | integrin | Laminin α1 L1 (A191) | |
| 439 | PGGMREKGRKAR | integrin | Laminin α1 L1 (A194) | |
| 440 | MEMQANLLLDRL | integrin | Laminin α1 L1 (A203) | |
| 441 | LSEIKLLISRAR | integrin | Laminin α1 L1 (A206) | |
| 442 | IKVAV | αvβ3 | Laminin α1 L1 (A208) | |
| 443 | AASIKVAVSADR | ανβ3 | Laminin α1 L1 (A208) | |
| 444 | NRWHSIYITRFG | α6β1 | Laminin α1 LG1 (AG10) | |
| 445 | SSFHFDGSGYAM | integrin | Laminin α1 LG2 (AG22) | |
| 446 | IAFQRN | α6β1 | Laminin α1 LG2 (AG32) | |
| 447 | TWYKIAFQRNRK | α6β1 | Laminin α1 LG2 (AG32) | |
| 448 | SLVRNRR VITIQ | integrin | Laminin α1 LG2 (AG56) | |
| 449 | DYATLQLQEGRLHFMF | α2β1 | Laminin EF-1 | |
| | DLG | | | |
| 450 | KKGSYNNIVVHV | integrin | Laminin α2 LG (A2G2) | |
| 451 | ADNLLFYLGSAK | integrin | Laminin α2 LG (A2G4) | |
| 452 | GSAKFIDFLAIE | integrin | Laminin α2 LG (A2G5) | |
| 453 | KVSFLWWVGSGV | integrin | Laminin α2 LG (A2G7) | |
| 454 | SYWYRIEASRTG | integrin | Laminin α2 LG (A2G10) | |
| 455 | ISTVMFKFRTFS | integrin | Laminin α2 LG (A2G25) | |
| 456 | KQANISIVDIDSN | integrin | Laminin α2 LG (A2G34) | |
| 457 | FSTRNESGIILL | integrin | Laminin α2 LG (A2G48) | |
| 458 | RRQTTQAYYAIF | integrin | Laminin α2 LG (A2G51) | |
| 459 | YAIFLNKGRLEV | integrin | Laminin α2 LG (A2G52) | |
| 460 | KNRLTIELEVRT | integrin | Laminin α2 LG (A2G76) | |
| 461 | GLLFYMARINHA | integrin | Laminin α2 LG (A2G78) | |
| 462 | VQLRNGFPYFSY | integrin | Laminin α2 LG (A2G80) | |
| 463 | HKIKIVRVKQEG | integrin | Laminin α2 LG (A2G84) | |
| 464 | DFGTVQLRNGFPFFSYD | integrin | Laminin EF-2 | |
| | LG | | | |
| 465 | YFDGTGFAKAVG | integrin | Laminin α2 LG (A2G94) | |
| 466 | NGQWHKVTAKKI | integrin | Laminin α2 LG (A2G103) | |
| 467 | AKKIKNRLELVV | integrin | Laminin α2 LG (A2G104) | |
| 468 | GFPGGLNQFGLTTN | integrin | Laminin α2 LG (A2G109) | |
| 469 | IRSLKLTKGTGKP | integrin | Laminin α2 LG (A2G111) | |
| 470 | AKALELRGVQPVS | integrin | Laminin α2 LG (A2G113) | |
| 471 | GQLFHVAYILIKF | integrin | Laminin α3 (A3-10) | |
| 472 | SQRIYQFAKLNYT | integrin | Laminin α3 LG (MA3G13) | |
| 473 | NVLSLYNFKTTF | integrin | Laminin α3 LG (MA3G22) | |
| 474 | NAPFPKLSWTIQ | integrin | Laminin α3 LG (MA3G27) | |
| 475 | WTIQTTVDRGLL | integrin | Laminin α3 LG (MA3G28) | |
| 476 | DTINNGRDHMILI | integrin | Laminin α3 LG (MA3G34) | |
| 477 | MILISIGKSQKRM | integrin | Laminin α3 LG (MA3G35) | |
| 478 | PPFLMLLKGSTR | integrin | Laminin α3 LG (A3GXX) | |
| 479 | NQRLASFSNAQQS | integrin | Laminin α3 LG (MA3G57) | |
| 480 | ISNVFVQRMSQSPEVLD | integrin | Laminin α3 LG (MA3G59) | |
| 481 | KARSFNVNOLLQD | integrin | Laminin α3 LG (MA3G63) | |
| 482 | KNSFMALYLSKG | integrin | Laminin α3 LG A3G75 | |
| 483 | KNSFMALYLSKGRLVF | integrin | Laminin α3 LG A3G756 | |
| | ALG | | | |
| 484 | RDSFVALYLSEGHVIFA | integrin | Laminin EF-3 | |
| | LG | | | |
| 485 | KPRLQFSLDIQT | integrin | Laminin α3 LG MA3G70 | |
| 486 | DGQWHSVTVSIK | integrin | Laminin α3 LG MA3G97 | |
| 487 | FVLYLGSKNAKK | integrin | Laminin α4 LG (A4G4) | |
| 488 | LAIKNDNLVYVY | integrin | Laminin α4 LG (A4G6) | |
| 489 | AYFSIVKIERVG | integrin | Laminin α4 LG (A4G10) | |
| 490 | DVISLYNFKHIY | integrin | Laminin α4 LG (A4G20) | |
| 491 | FFDGSSYAVVRD | integrin | Laminin α4 LG (A4G24) | |
| 492 | LHVFYDFGFSNG | integrin | Laminin α4 LG (A4G31) | |
| 493 | LKKAQINDAKYREISIIY | integrin | | |
| | HN | | | |
| 494 | RAYFNGQSFIAS | integrin | Laminin α4 LG (A4G47) | |
| 495 | SRLRGKNPTKGK | integrin | Laminin α4 LG (A4G59) | |
| 496 | LHKKGKNSSKPK | integrin | Laminin α4 LG (A4G69) | |
| 497 | RLKTRSSHGMIF | integrin | | |
| 498 | GEKSQFSIRLKT | integrin | Laminin α4 LG (A4G78) | |
| 499 | TLFLAHGRLVFM | integrin | Laminin α4 LG (A4G82) | |
| 500 | LVFMFNVGHKKL | integrin | Laminin α4 LG (A4G83) | |
| 501 | TLFLAHGRLVFMFNVG | integrin | Laminin α4 LG (A4G823) | |
| | HKKL | | | |
| 502 | DFMTLFLAHGRLVFMF | integrin | Laminin EF-4 | |
| | NVG | | | |
| 503 | HKKLKIRSQEKY | integrin | Laminin α4 LG (A4G84) | |
| 504 | GAAWKIKGPIYL | integrin | Laminin α4 LG (A4G90) | |
| 505 | VIRDSNVVQLDV | integrin | Laminin α4 LG (A4G107) | |
| 506 | EVNVTLDLGQVFH | α5β1 | Laminin Laminin α5 LN (S1) | |
| 507 | GQVFHVAYVLIKF | α4β1, α4β7 | Laminin Laminin α5 LN (S2) | |
| 508 | RDFTKATNIRLRFLR | α5β1 | Laminin Laminin α5 LN (S6) | |
| 509 | NIRLRFLRTNTL | α5β1 | Laminin Laminin α5 LN (S7) | |
| 510 | GKNTGDHFVLYM | α5β1 | Laminin α5 LG1 (A5G3) | |
| 511 | VVSLYNFEQTFML | integrin | Laminin α5 LG1 (A5G19) | |
| 512 | RFDQELRLVSYN | integrin | Laminin α5 LG2 (A5G26) | |
| 513 | ASKAIQVFLLGG | integrin | Laminin α5 LG2 (A5G33) | |
| 514 | TVFSVDQDNMLE | integrin | Laminin α5 LG2 (A5G36) | |
| 515 | RLRGPQRVFDLH | α5β1 | Laminin α5 LG3 (A5G63) | |
| 516 | SRATAQKVSRRS | integrin | Laminin α5 LG3 (A5G66) | |
| 517 | GSLSSHLEFVGI | integrin | Laminin α5 LG4 (A5G71) | |
| 518 | RNRLHLSMLVRP | integrin | Laminin α5 LG4 (A5G73) | |
| 519 | APMSGRSPSLVLK | integrin | Laminin α5 LG4 (A5G76) | |
| 520 | LALFLSNGHEVA | integrin | Laminin α5 LG4 (A5G77) | |
| 521 | PGRWHKVSVRWE | integrin | Laminin α5 LG4 (A5G81) | |
| 522 | VRWGMQQIQLVV | integrin | Laminin α5 LG4 (A5G82) | |
| 523 | KMPYVSLELEMR | integrin | Laminin α5 LG5 (A5G94) | |
| 524 | VLLQANDGAGEF | integrin | Laminin α5 LG5 (A5G99) | |
| 525 | DGRWHRVAVIMG | integrin | Laminin α5 LG5 (A5G101) | |
| 526 | APVNVTASVQIQ | integrin | Laminin α5 LG5 (A5G109) | |
| 527 | KOGKALTQRHAK | integrin | Laminin α5 LG5 (A5G112) | |
| 528 | AFGVLALWGTRV | integrin | Laminin Laminin VI (B-7) | |
| 529 | IENVVTTFAPNR | integrin | Laminin Laminin VI (B-15) | |
| 530 | LEAEFHFTHLIM | integrin | Laminin Laminin VI (B-19) | |
| 531 | HLIMTFKTFRPA | integrin | Laminin Laminin VI (B-20) | |
| 532 | KTWGVYRYFAYD | integrin | Laminin Laminin VI (B-23) | |
| 533 | TNLRIKFVKLHT | integrin | Laminin Laminin VI (B-31) | |
| 534 | REKYYYAVYDMV | integrin | Laminin Laminin VI (B-34) | |
| 535 | KRLVTGQR | integrin | Laminin Laminin V (B-54) | |
| 536 | KDISEKVAVYST | integrin | I (B-187) | |
| 537 | PDSGR | integrin | Laminin III (B-96) | |
| 538 | YIGSR | α1β1, α3β1 | Laminin III (B-98) | |
| 539 | DPGYIGSR | α1β1, α3β1 | Laminin III (B-98) | |
| 540 | FALWDAIIGEL | integrin | Laminin III (B-116) | |
| 541 | AAEPLKNIGILF | integrin | Laminin II (B-123) | |
| 542 | DSITKYFQMSLE | integrin | Laminin II (B-133) | |
| 543 | VILQQSAADIAR | integrin | Laminin I (B-160) | |
| 544 | SPYTFIDSLVLMPY | integrin | Laminin Laminin IV (B-77) | |
| 545 | KDISEKVAVYST | integrin | Laminin I (B-187) | |
| 546 | LGTIPG | integrin | | |
| 547 | LWPLLAVLAAVA | integrin | Laminin VI (C-3) | |
| 548 | KAFDITYVRLKF | αvβ3, α5β1 | Laminin VI (C-16) | |
| 549 | AFSTLEGRPSAY | integrin | Laminin VI (C-25) | |
| 550 | TDIRVTLNRLNTF | integrin | Laminin VI (C-28) | |
| 551 | NEPKVLKSYYYAI | integrin | Laminin VI (C-30) | |
| 552 | YYAISDFAVGGR | integrin | Laminin VI (C-31) | |
| 553 | LPFFNDRPWRRAT | integrin | Laminin VI (C-35) | |
| 554 | FDPELYRSTGHGGH | integrin | Laminin V (C-38) | |
| 555 | TNAVGYSVYDIS | integrin | Laminin V (C-50) | |
| 556 | APVKFLGNQVLSY | integrin | Laminin IV (C-57) | |
| 557 | SFSFRVDRRDTR | integrin | Laminin IV (C-59) | |
| 558 | SETTVKYIFRLHE | integrin | Laminin IV (C-64) | |
| 559 | FQKLLNNLTSIK | integrin | Laminin IV (C-67) | |
| 560 | TSIKIRGTYSER | integrin | Laminin IV (C-68) | |
| 561 | DPETGV | integrin | Laminin III (C75) | |
| 562 | TSAEAYNLLLRT | integrin | Laminin II (C-118) | |
| 563 | KEAEREVTDLLR | integrin | Laminin II (C102) | |
| 564 | SLLSQLNNLLDQ | integrin | Laminin II (C-155) | |
| 565 | RNIAEIIKDI | integrin | Laminin | |
| 566 | RDIAEIIKDI | integrin | Laminin | |
| 567 | GAPGER | integrin | Derived from Collagen alpha1(I) | |
| 568 | FNKHTEIIEEDTNKDKP | Fibronectin | (FAB D3: 1-37) - highest | Differential binding |
| | SYQFGGHNSVDFEEDT | | affinity | affinity to Collagen |
| | LPKV | | | |
| 569 | PSYQFGGHNSVDFEED | Fibronectin | (FAB D3: 16-36) - high | Differential binding |
| | TLPK | | affinity | affinity to Collagen |
| 570 | SYQFGGHNSVDFEEDT | Fibronectin | (FAB D3: 17-33) - medium | Differential binding |
| | | | affinity | affinity to Collagen |
| 571 | QFGGHNSVDFEEDTLP | Fibronectin | (FAB D3: 20-36) - medium | Differential binding |
| | K | | affinity | affinity to Collagen |
| 572 | FGGHNSVDFEEDTLPK | Fibronectin | (FAB D3: 21-36) - low | Differential binding |
| | | | affinity | affinity to Collagen |
| 573 | NAPQPSHISKYILRWRP | Fibronectin | Fibronectin Type III(1) | |
| | KNSVGRWKEATIPGHL | | | |
| | NSYTIKGLKPGVVYEG | | | |
| | QLISIQQYGHQEVTRFD | | | |
| | FTTTSTSTPVTSNTVTG | | | |
| | ETTPFSPLVATSESVTEI | | | |
| | TASSFVVS | | | |
| 574 | NAPQPSHISKYILRWRP | Fibronectin | Fibronectin Type III(1) | |
| | KNSVGRWKEATIPG | | fragment | |
| 575 | EATIPGHLNSYTIKGLK | Fibronectin | Fibronectin Type III(1) | |
| | PGVVYEGQLISIQQ | | fragment | |
| 576 | LISIQQYGHQEVTRFDF | Fibronectin | Fibronectin Type III(1) | |
| | TTTSTSTPVTSNTV | | fragment | |
| 577 | VTSNTVTGETTPFSPLV | Fibronectin | Fibronectin Type III(1) | |
| | ATSESVTEITASSFVVS | | fragment | |
| 578 | RWSHDNGVNYKIGEK | Fibronectin | Fibronectin Type III(1) fragment (synthetic) | |
| | WDRQGENGQMMSSTS | | | |
| | LGNGKGEFKSDPHE | | | |
| 579 | ATSYDDGKTYHVGEQ | Fibronectin | Fibronectin Type III(1) fragment (synthetic) | |
| | WQKEYLGAISSSTSFGG | | | |
| | QRGWRSDNSR | | | |
| 580 | DKPSYQFGGHNSVDFE | Fibronectin | | |
| | EDT | | | |
| 581 | DKPSYQFGGHNSVDFE | Fibronectin | | |
| | EDTL | | | |
| 582 | DKPSYQFGGHNSVDFE | Fibronectin | | |
| | EDTLP | | | |
| 583 | DKPSYQFGGHNSVDFE | Fibronectin | | |
| | EDTLPK | | | |
| 584 | KPSYQFGGHNSVDFEE | Fibronectin | | |
| | DT | | | |
| 585 | KPSYQFGGHNSVDFEE | Fibronectin | | |
| | DTL | | | |
| 586 | KPSYQFGGHNSVDFEE | Fibronectin | | |
| | DTLP | | | |
| 587 | KPSYQFGGHNSVDFEE | Fibronectin | | |
| | DTLPK | | | |
| 588 | PSYQFGGHNSVDFEED | Fibronectin | | |
| | T | | | |
| 589 | PSYQFGGHNSVDFEED | Fibronectin | | |
| | TL | | | |
| 590 | PSYQFGGHNSVDFEED | Fibronectin | | |
| | TLP | | | |
| 591 | PSYQFGGHNSVDFEED | Fibronectin | | |
| | TLPK | | | |
| 592 | PPFLMLLKGSTRFNKTK | Heparin/syndecans | Derived from Heparin Binding | Differential binding |
| | TFR | | Domans of Laminin | affinity to |
| | | | | Heparin/syndecans |
| 593 | RLVFALGTDGKKLRIKS | Heparin/syndecans | Derived from Heparin Binding | Differential binding |
| | KEKCNDGK | | Domans of Laminin | affinity to |
| | | | | Heparin/syndecans |
| 594 | PLFLLHKKGKNLSKPK | Heparin/syndecans | Derived from Heparin Binding | Differential binding |
| | ASQNKKGGKSK | | Domans of Laminin | affinity to |
| | | | | Heparin/syndecans |
| 595 | TLFLAHGRLVYMFNVG | Heparin/syndecans | Derived from Heparin Binding | Differential binding |
| | HKKLKIR | | Domans of Laminin | affinity to |
| | | | | Heparin/syndecans |
| 596 | TPGLGPRGLQATARKA | Heparin/syndecans | Derived from Heparin Binding | Differential binding |
| | SRRSRQPARHPACML | | Domans of Laminin | affinity to |
| | | | | Heparin/syndecans |
| 597 | RQRSRPGRWHKVSVR | Heparin/syndecans | Derived from Heparin Binding | Differential binding |
| | WEKNR | | Domans of Laminin | affinity to |
| | | | | Heparin/syndecans |
| 598 | LAGSCLARFSTM | a2B1, Heparin | Derived from Collagen alpha1(IV) HepII | |
| 599 | KGHRGF | Heparin | Derived from Collagen alpha1(I) | |
| 600 | GDRGIKGHRGFSG | Heparin | Derived from Collagen alpha1(I) | |
| 601 | GDLGRPGRKGRPGPP | Heparin | Derived from Collagen alpha1(I) | |
| 602 | GHRGPTGRPGKRGKQG | Heparin | Derived from Collagen alpha1(I) | |
| | QKGDS | | | |
| 603 | KGIRGH | Heparin | Derived from Collagen alpha2(I) | |
| 604 | GEFYFDLRLKGDK | α2β1, Heparin | Derived from Collagen alpha1(IV) HepIII | |
| 605 | KYILRWRPKNS | Heparin | Derived from Fibronectin III-1 | |
| 606 | YRVRVTPKEKTGPMKE | Heparin | Derived from Fibronectin III-13 (FN-C/H-III) | |
| 607 | SPPRRARVT | α5β1, Heparin | Derived from Fibronectin III-13 (FN-C/H-IV) | |
| 608 | ATETTITIS | Heparin | Derived from Fibronectin III-13 | |
| 609 | VSPPRRARVTDATETTI | α5β1, Heparin | Derived from Fibronectin III-13 | |
| | TISWRTKTETITGFG | | | |
| 610 | KPDVRSYTITG | α4β1, Heparin | Derived from Fibronectin III-13 | |
| 611 | ANGQTPIQRYIK | α4β1, Heparin | Derived from Fibronectin III-13 | |
| 612 | YEKPGSPPREVVPRPRP | Heparin | Derived from Fibronectin III-14 (FN-C/H-I) | |
| | GV | | | |
| 613 | KNNQKSEPLIGRKKT | Heparin | Derived from Fibronectin III-14 (FN-C/H-II) | |
| 614 | EILDVPST | integrin | Derived from Fibronectin IIICS-1 | |
| 615 | TAGSCLRKFSTM | α2B1, Heparin | Derived from Collagen alpha1(IV) HepI | |
| 616 | FRHRNRKGY | Heparin | HPV | |
| 617 | KKQRFRHRNRKGYRSQ | Heparin | HPV | |
| 618 | KRSR | Heparin | Bone sialoprotein | |
| 619 | FHRRIKA | Heparin, HSP | Bone sialoprotein | |
| 620 | SINNTAVMQRLT | Heparin | Laminin Laminin α1 L4a (A51) | |
| 621 | ANVTHLLIRANY | Heparin | Laminin α1 L4a (A65) | |
| 622 | AGTFALRGDNPQG | integrin | Laminin α1 L4a (A99) | |
| 623 | RLVSYSGVLFFLK | Heparin | Laminin α5 LG2 (A5G27) | |
| 624 | GIIFFL | Heparin | Laminin α5 LG2 (A5G) | |
| 625 | VLVRVERATVES | Heparin | Laminin α5 LG2 (A5G35) | |
| 626 | RIQNLLKITNLRIKFVK | Heparin | Laminin Laminin VI (B-30) | |
| 627 | GPGVVVVERQYI | Heparin | Laminin IV (B-62) | |
| 628 | RYVVLPR | Heparin | Laminin IV (B-73) | |
| 629 | LSNIDYILIKAS | SDC-4 | Laminin α1 L4a (A119) | |
| 630 | LQQSRIANISME | SDC-4 | Laminin α1 L4a (A121) | |
| 631 | LQVQLSIR | SDC-1, -4 | Laminin α1 LG4 (AG73) | |
| 632 | RKRLQVQLSIRT | SDC-1, -4 | Laminin α1 LG4 (AG73) | |
| 633 | GLIYYVAHQNQM | SDC-1, -4 | Laminin α1 LG4 (AG75) | |
| 634 | FDLHQNMGSVN | SDC-4 | Laminin α5 LG3 (A5G64) | |
| 635 | QQNLGSVNVSTG | SDC-4 | Laminin α5 LG3 (A5G65) | |
| 636 | WQPPRARI | SDC-4 | Derived from Fibronectin III-14 (FN-C/H-V) | |
| 637 | WQPPRARITGYIIKYEK | SDC-4 | Derived from Fibronectin III-14 (FN-C/H-V) | |
| | PG | | | |
| 638 | KNSFMALYLSKGR | syndecan 2(w) | Derived from Heparin Binding | Differential binding |
| | | | Domans of Laminin | affinity to |
| | | | | Heparin/syndecans |
| 639 | NGRKIRMRCRAIDGD | Heparan sulfate | binds to HSGP with high affinity (DTx protein) | |
| | | proteoglycans | | |
| 640 | DVIRDKTKTKIESLK | Heparan sulfate | binds to HSGP with low affinity (DTx protein) | |
| | | proteoglycans | | |

pH-sensitive targeting sequences

| 641 | GVYHREARSGKYKLTY | hyaluronic acid | pH dependent (Link_TGS6) | binds better at lower pH |
|---|---|---|---|---|
| | AEAKAVCEFEGGHLAT | | | |
| | YKGLEAARKIGFHVCA | | | |
| | AGWMAKGRVGYPIVK | | | |
| | PGPPNCGFGKTGIIDYGI | | | |
| | RLNRSERWDAYCYNPH | | | |
| | A | | | |
| 642 | KHAHLKKQVSDHIAVY | Heparin | binds to heparin at low pH (high affinity) | |
| 643 | TTEPSEEHNHHK | Heparin | binds to heparin at low pH (low affinity) | |
| 644 | KHAHL | Heparin | binds to heparin at low pH (lower affinity) | |
| 645 | TTEPSEEHNHHK | Heparin | binds to heparin at low pH (lower affinity) | |
| 646 | TTEPSEEHNHHKHHDK | Heparin | binds to heparin at low pH (lower affinity) | |
| 647 | HKGQHR | Heparin | binds to heparin at low pH (lower affinity) | |
| 648 | KVEHRVKKRPPTWRHN | Heparin | binds to heparin at low pH | |
| | VRAKYT | | | |
| 649 | GGKVEHRVKKRPPTWR | Heparin | binds to heparin at low pH | |
| | HNVRAKYT | | | |
| 650 | KKRPPTWRHNV | Heparin | binds to heparin at low pH | |
| 651 | GTWSEW | heparin | derived from thrombospondin | |
| 652 | GFWSEW | heparin | derived from thrombospondin | |
| 653 | GGWSHW | Fibronectin | derived from thrombospondin | binds better at lower pH |
| | | | (highest affinity) | |
| 654 | KRFKQDGGWSHWSPW | Fibronectin | derived from thrombospondin | |
| | SS | | (low affinity) | |

| | | | |
|---|---|---|---|
| 655 | KRFKQDGGWSHWSP | Fibronectin | derived from thrombospondin (medium affinity) |
| 656 | GGWSHWSPWSS | Fibronectin | derived from thrombospondin (medium affinity) |
| 657 | WSXWS | Sulfated | derived from thrombospondin (X = any amino acids) |
| | | Glycoprotein | |
| 658 | WSHW | Sulfated | derived from thrombospondin |
| | | Glycoprotein | |
| 659 | Xaa Xaa Pro His Glu | heparin/heparan | Xaa = any amino acid |
| | | sulfate | |
| 660 | (H/P)(H/P)PHG | heparin/heparan | tandem repeat - pH dependent HRGP (Histidine Rich |
| | | sulfate | Glyco Protein) |
| 661 | HPHKHHSHEQHPHGHH | heparin/heparan | Histidine Rich Glycoprotein (Histidine Rich Domain) |
| | PHAHHPHEHDTHRQHP | sulfate | |
| | HGHHPHGHHPHGHHPH | | |
| | GHHPHGHHPHCHDFQD | | |
| | YGPCDPPPHNQGHCCH | | |
| | GHGPPPGHLRRRGPGK | | |
| | GPRPFHCRQIGSVYRLP | | |
| | PLRKGEVLPLPEANFPS | | |
| | FPLPHHKHPLKPDNQPF | | |
| | P | | |
| 662 | DLHPHKHHSHEQHPHG | heparin/heparan | Histidine Rich Glycoprotein (Histidine Rich Domain) |
| | HHPHAHHPHEHDTHRQ | sulfate | |
| | HPH | | |
| 663 | GHHPH | heparin | |

Other targeting sequences

| | | |
|---|---|---|
| 664 | VRIQRKKEKMKET | heparin |
| 665 | LHERHLNNN | Collagen I |
| 666-673 | See Table 5 | |
| 680-700 | Not Used | |

### I. Definitions

As used herein, an “active domain” refers to a polypeptide or a collection of polypeptides that have affinity towards a target, which may be one or more polypeptides, nucleic acids, sugars, and/or combinations thereof. In some embodiments, an active domain is an agonist or antagonist of its target, or will bring about and/or inhibit signal transduction relating to the target. The active domain need not have exclusive affinity towards the target but instead only needs to have affinity towards the target that is significantly higher (e.g., 10 times or more) than the domain's affinity towards a non-target. A dissociation constant (K_{D}) between a active domain and a target may be in the range of pM, nM, μM, or mM. An active domain may comprise one or more subdomains or subunits that each has distinctive functions and together have the function of the active domain. For example, an active domain that comprises an IL-12 polypeptide sequence may comprise two subunits.

As used herein, an “immunoglobulin antigen-binding domain” refers to a domain that is an immunoglobulin or a fragment thereof, such as an Fv, scFv, Fab, or VHH. Exemplary immunoglobulin antigen-binding domains are provided in Table 1.

As used herein, a “receptor-binding domain” refers to an active domain, such as a cytokine polypeptide sequence, that is not an immunoglobulin antigen-binding domain.

As used herein, a “cytokine polypeptide sequence” refers to a polypeptide sequence (which may be part of a larger sequence, e.g., a fusion polypeptide) with significant sequence identity to a wild-type cytokine and which can bind and activate a cytokine receptor (e.g., when separated from an inhibitory polypeptide sequence). In some embodiments, a cytokine polypeptide sequence has at least 80, 85, 90, 95, 97, 98, or 99 percent identity to the sequence of a wild-type cytokine, e.g., a wild-type human cytokine. In some embodiments, a cytokine polypeptide sequence has no more than one, two, three, four, five, six, seven, eight, nine, or ten amino acid differences from a wild-type cytokine, e.g., a wild-type human cytokine. Cytokines include but are not limited to chemokines. Exemplary cytokine polypeptide sequences are provided in Table 1. This definition applies to IL-2 polypeptide sequences with substitution of “IL-2” for “cytokine.”

As used herein, an “inhibitory polypeptide sequence” refers to a polypeptide or a collection of polypeptides that inhibits an activity of an active domain in the linker polypeptide. The inhibitory polypeptide sequence may bind or sterically obstruct the active domain. In some embodiments, such binding is reduced or eliminated by action of an appropriate protease on a protease-cleavable polypeptide sequence of the linker polypeptide. Exemplary inhibitory polypeptide sequences are provided in Table 1. The inhibitory polypeptide sequence may, for example, comprise a polypeptide with significant sequence identity to a part of a wild-type target of an active domain, or an immunoglobulin or a fraction thereof, such as an Fv, scFv, Fab, or VHH.

As used herein, a “protease-cleavable polypeptide sequence” is a sequence that is a substrate for cleavage by a protease. The protease-cleavable polypeptide sequence is located in a linker polypeptide such that its cleavage releases one or more elements of the linker polypeptide from the remainder of the linker polypeptide, or reduces or eliminates binding of an inhibitory polypeptide sequence to an active domain.

As used herein, a protease-cleavable polypeptide sequence “is recognized by” a given protease or class thereof if exposing a polypeptide comprising the protease-cleavable polypeptide sequence to the protease under conditions permissive for cleavage by the protease results in a significantly greater amount of cleavage than is seen for a control polypeptide having an unrelated sequence, and/or if the protease-cleavable polypeptide sequence corresponds to a known recognition sequence for the protease (e.g., as described elsewhere herein for various exemplary proteases).

As used herein, a “pharmacokinetic modulator” is a moiety that extends the in vivo half-life of a linker polypeptide or an element of the linker polypeptide. The pharmacokinetic modulator may be a fused domain in a linker polypeptide or may be a chemical entity attached post-translationally. The attachment may be, but is not necessarily, covalent. Exemplary pharmacokinetic modulator polypeptide sequences are provided in Table 1. Exemplary non-polypeptide pharmacokinetic modulators are described elsewhere herein.

As used herein, a “targeting sequence” is a sequence that results in a greater fraction of a linker polypeptide localizing to an area of interest, e.g., a tumor microenvironment. The targeting sequence may bind an extracellular matrix component or other entity found in the area of interest, e.g., an integrin or syndecan. Exemplary targeting sequences are provided in Table 2.

As used herein, an “extracellular matrix component” refers to an extracellular protein or polysaccharide found in vivo. Integral and peripheral membrane proteins on a cell, including fibronectins, cadherins, integrins, and syndecans, are not considered extracellular matrix components.

As used herein, an “immunoglobulin constant domain” refers to a domain that occurs in or has significant sequence identity to a domain of a constant region of an immunoglobulin, such as an IgG. Exemplary constant domains are C_{H}2 and C_{H}3 domains. Unless indicated otherwise, a linker polypeptide comprising an immunoglobulin constant domain may comprise more than one immunoglobulin constant domain. In some embodiments, an immunoglobulin constant domain has at least 80, 85, 90, 95, 97, 98, or 99 percent identity to the sequence of a wild-type immunoglobulin constant domain, e.g., a wild-type human immunoglobulin constant domain. In some embodiments, an immunoglobulin constant domain has no more than one, two, three, four, five, six, seven, eight, nine, or ten amino acid differences from a wild-type immunoglobulin constant domain, e.g., a wild-type human immunoglobulin constant domain. In some embodiments, immunoglobulin constant domain has an identical sequence to a wild-type immunoglobulin constant domain, e.g., a wild-type human immunoglobulin constant domain. Exemplary immunoglobulin constant domains are contained within sequences provided in Table 1. This definition applies to C_{H}2 and C_{H}3 domains, respectively, with substitution of “C_{H}2” or “C_{H}3” for “immunoglobulin constant,” with the qualification that a C_{H}2 domain sequence does not have greater percent identity to a non-C_{H}2 immunoglobulin constant domain wild-type sequence than to a C_{H}2 domain wild-type sequence, and a C_{H}3 domain sequence does not have greater percent identity to a non-C_{H}3 immunoglobulin constant domain wild-type sequence than to a C_{H}3 domain wild-type sequence. These definitions also include domains having minor truncations relative to wild-type sequences, to the extent that the truncation does not abrogate substantially normal folding of the domain.

As used herein, a “immunoglobulin Fc region” refers to a region of an immunoglobulin heavy chain comprising a C_{H}2 and a C_{H}3 domain, as defined above. The Fc region does not include a variable domain or a C_{H}1 domain.

As used herein, a given component is “between” a first component and a second component if the first component is on one side of the given component and the second component is on the other side of the given component, e.g., in the primary sequence of a polypeptide. This term does not require immediate adjacency. Thus, in the structure 1-2-3-4, 2 is between 1 and 4, and is also between 1 and 3.

As used herein, a “domain” may refer, depending on the context, to a structural domain of a polypeptide or to a functional assembly of at least one domain (but possibly a plurality of structural domains). For example, a C_{H}2 domain refers to a part of a sequence that qualifies as such. An immunoglobulin cytokine-binding domain may comprise VH and VL structural domains.

As used herein, “denatured collagen” encompasses gelatin and cleavage products resulting from action of an MMP on collagen, and more generally refers to a form of collagen or fragments thereof that does not exist in the native structure of full-length collagen.

As used herein, “configured to bind . . . in a pH-sensitive manner” means that a polypeptide sequence (e.g., a targeting sequence) shows differential binding affinity for its binding partner depending on pH. For example, the polypeptide sequence may have a higher affinity at a relatively acidic pH than at normal physiological pH (about 7.4). The higher affinity may occur at a pH below 7, e.g., in the range of pH 5.5-7, 6-7, or 5.5-6.5, or below pH 6.

As used herein, a “cytokine-binding domain of a cytokine receptor” refers to an extracellular portion of a cytokine receptor, or a fragment or truncation thereof that can bind a cytokine polypeptide sequence. In some embodiments, the sequence of a cytokine binding domain of a cytokine receptor has at least 80, 85, 90, 95, 97, 98, or 99 percent identity to the sequence of a cytokine binding domain of a wild-type cytokine receptor, e.g., a cytokine binding domain of a wild-type human cytokine receptor. Exemplary sequences of a cytokine binding domain of a cytokine receptor are provided in Table 1. This definition applies to IL-2, IL-10, IL-15, CXCL9, CXCL10, and TGF-β-binding domains of an IL-2, IL-10, IL-15, CXCL9, CXCL10, and TGF-β receptor with substitution of “IL-2,” “IL-10,” “IL-15,” “CXCL9,” “CXCL10,” and “TGF-β,” respectively, for “cytokine.”

As used herein, an “immunoglobulin cytokine-binding domain” refers to one or more immunoglobulin variable domains (e.g., a VH and a VL region) that can bind a cytokine polypeptide sequence. Exemplary sequences of a cytokine-binding immunoglobulin domain are provided in Table 1. This definition applies to IL-2, IL-10, IL-15, CXCL9, CXCL10, and TGF-β-binding domains of an IL-2, IL-10, IL-15, CXCL9, CXCL10, and TGF-β receptor with substitution of “IL-2,” “IL-10,” “IL-15,” “CXCL9,” “CXCL10,” and “TGF-β,” respectively, for “cytokine.”

As used herein, a first element of the linker polypeptide being “proximal to” a second element relative to a third element means that in the primary polypeptide sequence of the linker polypeptide, the first element is closer to the second element than to the third element, regardless of whether the first element is spacially closer to the second element than to the third element when the linker polypeptide is folded.

As used herein, “substantially” and other grammatical forms thereof mean sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “plurality” can be 2, 3, 4, 5, 6, 7, 8, 9, 10, or more.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence QLYV (SEQ ID NO: 1168) comprises a sequence with 100% identity to the sequence QLY because an alignment would give 100% identity in that there are matches to all three positions of the second sequence. Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

As used herein, a “subject” refers to any member of the animal kingdom. In some embodiments, “subject” refers to humans. In some embodiments, “subject” refers to non-human animals. In some embodiments, “subject” refers to primates. In some embodiments, subjects include, but are not limited to, mammals, birds, reptiles, amphibians, fish, insects, and/or worms. In certain embodiments, the non-human subject is a mammal (e.g., a rodent, a mouse, a rat, a rabbit, a monkey, a dog, a cat, a sheep, cattle, a primate, and/or a pig). In some embodiments, a subject may be a transgenic animal, genetically-engineered animal, and/or a clone. In certain embodiments of the present invention the subject is an adult, an adolescent or an infant. In some embodiments, the terms “individual” or “patient” are used and are intended to be interchangeable with “subject”.

### II. Linker Polypeptide

The linker polypeptide may comprise a first targeting sequence; a second targeting sequence; and a first linker between the first targeting sequence and the second targeting sequence, the linker comprising a protease-cleavable polypeptide sequence. In some embodiments, the first targeting sequence and/or the second targeting sequence may each comprise two or more targeting subsequences that each binds to a target. In some embodiments, some or all of the two or more targeting subsequences may bind to the same target (e.g., tandem repeats). In some embodiments, the linker polypeptide comprises a first active domain; a second active domain; a pharmacokinetic modulator; and a first linker between the pharmacokinetic modulator and the first active domain, the first linker comprising a protease-cleavable polypeptide sequence. In some embodiments, the linker polypeptide comprises a first active domain; an inhibitory polypeptide sequence capable of blocking an activity of the first active domain; a first linker between the first active domain and the inhibitory polypeptide sequence, the linker comprising a protease-cleavable polypeptide sequence; and a first targeting sequence.

These elements of the linker polypeptide may be covalently connected to form a single polypeptide chain or may be present in a plurality of associated polypeptide chains, which may be linked noncovalently or covalently (e.g., via one or more disulfide bonds).

In some embodiments, the linker polypeptide comprises a first polypeptide chain comprising a first active domain, a first domain of a pharmacokinetic modulator, and a first linker between the first active domain and the first domain of the pharmacokinetic modulator, wherein the first active domain is C-terminal to the first domain of the pharmacokinetic modulator; a second polypeptide chain, comprising a second domain of the pharmacokinetic modulator, an inhibitory polypeptide sequence capable of blocking an activity of the first active domain, and a second linker between the second domain of the pharmacokinetic modulator and the inhibitory polypeptide sequence; wherein the first linker comprises a protease-cleavable polypeptide sequence; and the first polypeptide chain or the second polypeptide chain further comprises at least one targeting sequence.

In some embodiments, the linker polypeptide comprises a first polypeptide chain comprising a first active domain, a first domain of a pharmacokinetic modulator, and a first linker between the first active domain and the first domain of the pharmacokinetic modulator, wherein the first active domain is N-terminal to the first domain of the pharmacokinetic modulator; a second polypeptide chain, comprising a second domain of the pharmacokinetic modulator, an inhibitory polypeptide sequence capable of blocking an activity of the first active domain, and a second linker between the second domain of the pharmacokinetic modulator and the inhibitory polypeptide sequence; wherein the first linker comprises a protease-cleavable polypeptide sequence; and the first polypeptide chain or the second polypeptide chain further comprises at least one targeting sequence.

### A. Active Domain

### 1. Immunoglobulin Antigen-Binding Domain

In some embodiments, the first active domain comprises an immunoglobulin antigen-binding domain. In some embodiments, the second active domain comprises an immunoglobulin antigen-binding domain.

In some embodiments, the immunoglobulin antigen-binding domain comprises a VH region and a VL region. In some embodiments, the immunoglobulin antigen-binding domain comprises an Fv, scFv, Fab, or VHH. The immunoglobulin antigen-binding domain may be humanized or fully human.

In some embodiments, the immunoglobulin antigen-binding domain binds to one or more sequences selected from a cancer cell surface antigen sequence, a growth factor sequence, and a growth factor receptor sequence.

Under physiological conditions, cells receive signals from surrounding tissue in the form of growth factors. Growth factors can influence normal cell differentiation as well as constitutively activate growth-promoting pathways in cancer cells. The linker polypeptides disclosed herein may bind to growth factors to facilitate neutralization of the activity of the growth factor to at least some extent, e.g., in the vicinity of a tumor. Thus, the linker polypeptides disclosed here, through an immunoglobulin antigen-binding domain, can in some embodiments reduce the pro-growth signaling received by cancer cells and stromal cells, including fibroblast and endothelial cells, while also activating or recruiting immune cells to the tumor. In some embodiments, the immunoglobulin antigen-binding domain may also promote localization of linker polypeptides to tissues that specifically express particular growth factors or tissues that express particular growth factors in high amounts, e.g., in and around tumors.

Growth factor receptors are generally transmembrane proteins that bind to specific growth factors and transmit the instructions conveyed by the factors on the outside of a cell to intracellular space. In general, growth factor receptors comprise extracellular, transmembrane, and cytoplasmic domains. In some embodiments, the linker polypeptides disclosed here, through an immunoglobulin antigen-binding domain, can inhibit binding of a growth factor to the growth factor receptor. This may facilitate reduction of signaling by the growth factor to at least some extent, e.g., in the vicinity of a tumor.

In some embodiments, one or each of the first immunoglobulin antigen-binding domain and the second immunoglobulin antigen-binding domain of the linker polypeptide independently is configured to bind to a HER2 sequence. In some embodiments, one or each of the first immunoglobulin antigen-binding domain and the second immunoglobulin antigen-binding domain of the linker polypeptide independently comprises hypervariable regions (HVRs) HVR-1, HVR-2, and HVR-3 of a VH region comprising the amino acid sequence of SEQ ID NO: 910, and a VL region comprising HVR-1, HVR-2, and HVR-3 of a VL region comprising the amino acid sequence of SEQ ID NO: 909. In general, a person skilled in the art can identify the HVRs in VH and VL sequences, e.g., by assigning amino acids to framework and HVR domains within the VH and VL sequences in accordance with the definitions of Kabat et al. in Sequences of Proteins of Immunological Interest, 5^{th} Ed., US Dept. of Health and Human Services, PHS, NIH, NIH Publication no. 91-3242, 1991. Other numbering systems for the amino acids in immunoglobulin chains include IMGT™ (international ImMunoGeneTics information system; Lefranc et al, Dev. Comp. Immunol. 29:185-203; 2005) and AHo (Honegger and Pluckthun, J. Mol. Biol. 309(3):657-670; 2001). In some embodiments, one or each of the first immunoglobulin antigen-binding domain and the second immunoglobulin antigen-binding domain independently comprises a VH region comprising the amino acid sequence of SEQ ID NO: 910; and a VL region comprising the amino acid sequence of SEQ ID NO: 909. In some embodiments, one or each of the first immunoglobulin antigen-binding domain and the second immunoglobulin antigen-binding domain independently comprises a sequence that has at least 80, 85, 90, 95, 97, 98, or 99 percent identity to the sequence of SEQ ID NO: 909 or 910. In some embodiments, one or each of the first immunoglobulin antigen-binding domain and the second immunoglobulin antigen-binding domain independently is an antigen-binding domain of trastuzumab.

In some embodiments, one or each of the first immunoglobulin antigen-binding domain and the second immunoglobulin antigen-binding domain of the linker polypeptide independently is configured to bind to an EGFR extracellular domain sequence. In some embodiments, each of the first immunoglobulin antigen-binding domain and the second immunoglobulin antigen-binding domain independently comprises a VH region comprising HVR-1, HVR-2, and HVR-3 of a VH region comprising the amino acid sequence of SEQ ID NO: 914, and a VL region comprising HVR-1, HVR-2, and HVR-3 of a VL region comprising the amino acid sequence of SEQ ID NO: 913. In general, a person skilled in the art can identify the HVRs in VH and VL sequences, e.g., by assigning amino acids to framework and HVR domains within the VH and VL sequences in accordance with the definitions of Kabat et al. in Sequences of Proteins of Immunological Interest, 5^{th} Ed., US Dept. of Health and Human Services, PHS, NIH, NIH Publication no. 91-3242, 1991. Other numbering systems for the amino acids in immunoglobulin chains include IMGT™ (international ImMunoGeneTics information system; Lefranc et al, Dev. Comp. Immunol. 29:185-203; 2005) and AHo (Honegger and Pluckthun, J. Mol. Biol. 309(3):657-670; 2001). In some embodiments, one or each of the first immunoglobulin antigen-binding domain and the second immunoglobulin antigen-binding domain independently comprises a VH region comprising the amino acid sequence of SEQ ID NO: 914; and a VL region comprising the amino acid sequence of SEQ ID NO: 913. In some embodiments, one or each of the first immunoglobulin antigen-binding domain and the second immunoglobulin antigen-binding domain independently comprises a sequence that has at least 80, 85, 90, 95, 97, 98, or 99 percent identity to the sequence of SEQ ID NO: 913 or 914. In some embodiments, one or each of the first immunoglobulin antigen-binding domain and the second immunoglobulin antigen-binding domain is an antigen-binding domain of cetuximab.

In some embodiments, one or each of the first immunoglobulin antigen-binding domain and the second immunoglobulin antigen-binding domain of the linker polypeptide independently is configured to bind to a PD-1 extracellular domain sequence. In some embodiments, one or each of the first immunoglobulin antigen-binding domain and the second immunoglobulin antigen-binding domain independently comprises a VH region comprising HVR-1, HVR-2, and HVR-3 of a VH region comprising the amino acid sequence of SEQ ID NO: 917, and a VL region comprising HVR-1, HVR-2, and HVR-3 of a VL region comprising the amino acid sequence of SEQ ID NO: 918. In general, a person skilled in the art can identify the HVRs in VH and VL sequences, e.g., by assigning amino acids to framework and HVR domains within the VH and VL sequences in accordance with the definitions of Kabat et al. in Sequences of Proteins of Immunological Interest, 5^{th} Ed., US Dept. of Health and Human Services, PHS, NIH, NIH Publication no. 91-3242, 1991. Other numbering systems for the amino acids in immunoglobulin chains include IMGT™ (international ImMunoGeneTics information system; Lefranc et al, Dev. Comp. Immunol. 29:185-203; 2005) and AHo (Honegger and Pluckthun, J. Mol. Biol. 309(3):657-670; 2001). In some embodiments, one or each of the first immunoglobulin antigen-binding domain and the second immunoglobulin antigen-binding domain independently comprises a VH region comprising the amino acid sequence of SEQ ID NO: 917; and a VL region comprising the amino acid sequence of SEQ ID NO: 918. In some embodiments, one or each of the first immunoglobulin antigen-binding domain and the second immunoglobulin antigen-binding domain independently comprises a sequence that has at least 80, 85, 90, 95, 97, 98, or 99 percent identity to the sequence of SEQ ID NO: 917 or 918. In some embodiments, one or each of the first immunoglobulin antigen-binding domain and the second immunoglobulin antigen-binding domain independently is an antigen-binding domain of nivolumab.

In some embodiments, one or each of the first immunoglobulin antigen-binding domain and the second immunoglobulin antigen-binding domain of the linker polypeptide independently is configured to bind to a PD-L1 extracellular domain sequence. In some embodiments, one or each of the first immunoglobulin antigen-binding domain and the second immunoglobulin antigen-binding domain independently comprises a VH region comprising HVR-1, HVR-2, and HVR-3 of a VH region comprising the amino acid sequence of SEQ ID NO: 921, and a VL region comprising HVR-1, HVR-2, and HVR-3 of a VL region comprising the amino acid sequence of SEQ ID NO: 922. In general, a person skilled in the art can identify the HVRs in VH and VL sequences, e.g., by assigning amino acids to framework and HVR domains within the VH and VL sequences in accordance with the definitions of Kabat et al. in Sequences of Proteins of Immunological Interest, 5^{th} Ed., US Dept. of Health and Human Services, PHS, NIH, NIH Publication no. 91-3242, 1991. Other numbering systems for the amino acids in immunoglobulin chains include IMGT™ (international ImMunoGeneTics information system; Lefranc et al, Dev. Comp. Immunol. 29:185-203; 2005) and AHo (Honegger and Pluckthun, J. Mol. Biol. 309(3):657-670; 2001). In some embodiments, one or each of the first immunoglobulin antigen-binding domain and the second immunoglobulin antigen-binding domain independently comprises a VH region comprising the amino acid sequence of SEQ ID NO: 921; and a VL region comprising the amino acid sequence of SEQ ID NO: 922. In some embodiments, one or each of the first immunoglobulin antigen-binding domain and the second immunoglobulin antigen-binding domain independently comprises a sequence that has at least 80, 85, 90, 95, 97, 98, or 99 percent identity to the sequence of SEQ ID NO: 921 or 922. In some embodiments, one or each of the first immunoglobulin antigen-binding domain and the second immunoglobulin antigen-binding domain independently is an antigen-binding domain of atezolizumab.

In some embodiments, one or each of the first immunoglobulin antigen-binding domain and the second immunoglobulin antigen-binding domain of the linker poly peptide independently is configured to bind to a CD3 extracellular domain sequence. In some embodiments, one or each of the first immunoglobulin antigen-binding domain and the second immunoglobulin antigen-binding domain independently comprises a VH region comprising HVR-1, HVR-2, and HVR-3 of a VH region comprising the amino acid sequence of any one of SEQ ID NOs: 925, 929, 933, and 937, and a VL region comprising HVR-1, HVR-2, and HVR-3 of a VL region comprising the amino acid sequence of any one of SEQ ID NOs: 926, 930, 934, and 938. In general, a person skilled in the art can identify the HVRs in VH and VL sequences, e.g., by assigning amino acids to framework and HVR domains within the VH and VL sequences in accordance with the definitions of Kabat et al. in Sequences of Proteins of Immunological Interest, 5^{th} Ed., US Dept. of Health and Human Services, PHS, NIH, NIH Publication no. 91-3242, 1991. Other numbering systems for the amino acids in immunoglobulin chains include IMGT™ (international ImMunoGeneTics information system; Lefranc et al, Dev. Comp. Immunol. 29:185-203; 2005) and AHo (Honegger and Pluckthun, J. Mol. Biol. 309(3):657-670; 2001). In some embodiments, one or each of the first immunoglobulin antigen-binding domain and the second immunoglobulin antigen-binding domain independently comprises a VH region comprising the amino acid sequence of any one of SEQ ID NOs: 925, 929, 933, and 937; and a VL region comprising the amino acid sequence of any one of SEQ ID NOs: 926, 930, 934, and 938. In some embodiments, one or each of the first immunoglobulin antigen-binding domain and the second immunoglobulin antigen-binding domain independently comprises a sequence that has at least 80, 85, 90, 95, 97, 98, or 99 percent identity to the sequence of any one of SEQ ID NOs: 925, 926, 929, 930, 933, 934, 937, and 938. In some embodiments, one or each of the first immunoglobulin antigen-binding domain and the second immunoglobulin antigen-binding domain independently is an antigen-binding domain of teplizumab, muromonab, otelixizumab, or visilizumab.

### 2. Receptor-Binding Domain

In some embodiments, the first active domain comprises a receptor-binding domain. The receptor-binding domain may comprise, for example, a cytokine polypeptide sequence.

The receptor-binding domain may be a wild-type receptor-binding domain or a sequence with one or more differences from the wild-type receptor-binding domain. In some embodiments, the receptor-binding domain is a human receptor-binding domain (which may be wild-type or may have one or more differences). In some embodiments, the receptor-binding domain comprises a modification to prevent disulfide bond formation, and optionally otherwise comprises wild-type sequence. In some embodiments, the receptor-binding domain has at least 80, 85, 90, 95, 97, 98, or 99 percent identity to the sequence of a wild-type receptor-binding domain or to a receptor-binding domain in Table 1. In some embodiments, the receptor-binding domain is a dimeric receptor-binding domain, e.g., a heterodimeric cytokine. In some embodiments, the receptor-binding domain is a homodimeric receptor-binding domain, e.g., a homodimeric cytokine. The monomers may be linked as a fusion protein, e.g., with a linker, or by a covalent bond (e.g., disulfide bond), or by a noncovalent interaction. In some embodiments, the receptor-binding domain is an interleukin polypeptide sequence. In some embodiments, the receptor-binding domain is capable of binding a receptor comprising CD132. In some embodiments, the receptor-binding domain is capable of binding a receptor comprising CD122. In some embodiments, the receptor-binding domain is capable of binding a receptor comprising CD25.

In some embodiments, the receptor-binding domain is an IL-2 polypeptide sequence. The IL-2 polypeptide sequence may be a wild-type IL-2 polypeptide sequence or a sequence with one or more differences from the wild-type IL-2 polypeptide sequence. In some embodiments, the IL-2 polypeptide sequence is a human IL-2 polypeptide sequence (which may be wild-type or may have one or more differences). In some embodiments, the IL-2 comprises a modification to prevent disulfide bond formation (e.g., the sequence of aldesleukin (marketed as Proleukin®), and optionally otherwise comprises wild-type sequence. In some embodiments, the IL-2 polypeptide sequence has at least 80, 85, 90, 95, 97, 98, or 99 percent identity to the sequence of a wild-type IL-2 polypeptide sequence or to an IL-2 polypeptide sequence in Table 1.

In some embodiments, the IL-2 polypeptide sequence comprises the sequence of any one of SEQ ID NOs: 1-4. In some embodiments, the IL-2 polypeptide sequence comprises the sequence of SEQ ID NO: 1. In some embodiments, the IL-2 polypeptide sequence comprises the sequence of SEQ ID NO: 2.

In some embodiments, the receptor-binding domain is an IL-10 polypeptide sequence. The IL-10 polypeptide sequence may be a wild-type IL-10 polypeptide sequence or a sequence with one or more differences from the wild-type IL-10 polypeptide sequence. In some embodiments, the IL-10 polypeptide sequence is a human IL-10 polypeptide sequence (which may be wild-type or may have one or more differences). In some embodiments, the IL-10 comprises a modification to prevent disulfide bond formation, and optionally otherwise comprises wild-type sequence. In some embodiments, the IL-10 polypeptide sequence has at least 80, 85, 90, 95, 97, 98, or 99 percent identity to the sequence of a wild-type IL-10 polypeptide sequence or to an IL-10 polypeptide sequence in Table 1. In some embodiments, the IL-10 polypeptide sequence comprises the sequence of SEQ ID NO: 900.

In some embodiments, the receptor-binding domain is an IL-15 polypeptide sequence. The IL-15 polypeptide sequence may be a wild-type IL-15 polypeptide sequence or a sequence with one or more differences from the wild-type IL-15 polypeptide sequence. In some embodiments, the IL-15 polypeptide sequence is a human IL-15 polypeptide sequence (which may be wild-type or may have one or more differences). In some embodiments, the IL-15 comprises a modification to prevent disulfide bond formation, and optionally otherwise comprises wild-type sequence. In some embodiments, the IL-15 polypeptide sequence has at least 80, 85, 90, 95, 97, 98, or 99 percent identity to the sequence of a wild-type IL-15 polypeptide sequence or to an IL-15 polypeptide sequence in Table 1. In some embodiments, the IL-15 polypeptide sequence comprises the sequence of SEQ ID NO: 901.

In some embodiments, the receptor-binding domain is an CXCL9 polypeptide sequence. The CXCL9 polypeptide sequence may be a wild-type CXCL9 polypeptide sequence or a sequence with one or more differences from the wild-type CXCL9 polypeptide sequence. In some embodiments, the CXCL9 polypeptide sequence is a human CXCL9 polypeptide sequence (which may be wild-type or may have one or more differences). In some embodiments, the CXCL9 comprises a modification to prevent disulfide bond formation, and optionally otherwise comprises wild-type sequence. In some embodiments, the CXCL9 polypeptide sequence has at least 80, 85, 90, 95, 97, 98, or 99 percent identity to the sequence of a wild-type CXCL9 polypeptide sequence or to an CXCL9 polypeptide sequence in Table 1. In some embodiments, the CXCL9 polypeptide sequence comprises the sequence of SEQ ID NO: 902.

In some embodiments, the receptor-binding domain is an CXCL10 polypeptide sequence. The CXCL10 polypeptide sequence may be a wild-type CXCL10 polypeptide sequence or a sequence with one or more differences from the wild-type CXCL10 polypeptide sequence. In some embodiments, the CXCL10 polypeptide sequence is a human CXCL10 polypeptide sequence (which may be wild-type or may have one or more differences). In some embodiments, the CXCL10 comprises a modification to prevent disulfide bond formation, and optionally otherwise comprises wild-type sequence. In some embodiments, the CXCL10 polypeptide sequence has at least 80, 85, 90, 95, 97, 98, or 99 percent identity to the sequence of a wild-type CXCL10 polypeptide sequence or to an CXCL10 polypeptide sequence in Table 1. In some embodiments, the CXCL10 polypeptide sequence comprises the sequence of SEQ ID NO: 903.

### 3. Size of Active Domain

In some embodiments, a molecular weight of one or each of the first active domain and the second active domain independently is about or less than 14 kDa. In some embodiments, the molecular weight is about 12 kDa to about 14 kDa. In some embodiments, the molecular weight is about 10 kDa to about 12 kDa. In some embodiments, the molecular weight is about 8 kDa to about 10 kDa. In some embodiments, the molecular weight is about 6 kDa to about 8 kDa. In some embodiments, the molecular weight is about 4 kDa to about 6 kDa. In some embodiments, the molecular weight is about 2 kDa to about 4 kDa. In some embodiments, the molecular weight is about 800 Da to about 2 kDa.

In some embodiments, the molecular weight of one or each of the first active domain and the second active domain independently is about or greater than 16 kDa. In some embodiments, the molecular weight is about 16 kDa to about 18 kDa. In some embodiments, the molecular weight is about 18 kDa to about 20 kDa. In some embodiments, the molecular weight is about 20 kDa to about 22 kDa. In some embodiments, the molecular weight is about 22 kDa to about 24 kDa. In some embodiments, the molecular weight is about 24 kDa to about 26 kDa. In some embodiments, the molecular weight is about 26 kDa to about 28 kDa. In some embodiments, the molecular weight is about 28 kDa to about 30 kDa. In some embodiments, the molecular weight is about 30 kDa to about 50 kDa. In some embodiments, the molecular weight is about 50 kDa to about 100 kDa. In some embodiments, the molecular weight is about 100 kDa to about 150 kDa. In some embodiments, the molecular weight is about 150 kDa to about 200 kDa. In some embodiments, the molecular weight is about 200 kDa to about 250 kDa. In some embodiments, the molecular weight is about 250 kDa to about 300 kDa.

### B. Inhibitory Polypeptide Sequence

In some embodiments, the linker polypeptide comprises an inhibitory polypeptide sequence capable of blocking an activity of an active domain, such as a receptor-binding domain. In some embodiments, the linker polypeptide further comprises a second linker between the receptor-binding domain and the inhibitory polypeptide sequence, the second linker comprising a protease-cleavable polypeptide sequence.

Various types of inhibitory polypeptide sequences may be used in a linker polypeptide according to the disclosure. In some embodiments, the inhibitory polypeptide sequence is a sequence that binds the active domain, such as a ligand-binding domain from a receptor, or an immunoglobulin domain. In some embodiments, the inhibitory polypeptide sequence is a steric blocker, i.e., a sequence that sterically obstructs the active domain. For example, a steric blocker can be an immunoglobulin Fc region, an albumin domain, or other relatively inert domain, which can be placed in proximity to the active domain to render it less accessible until the active domain is liberated from the inhibitory polypeptide sequence by cleavage. In some embodiments, the inhibitory polypeptide sequence interferes with binding between the first active domain and a receptor of the first active domain and/or with binding between the second active domain and a receptor of the second active domain. In some embodiments, the inhibitory polypeptide sequence and the pharmacokinetic modulator are different elements of the linker polypeptide. In some embodiments, the inhibitory polypeptide sequence comprises at least a portion of the pharmacokinetic modulator.

In some embodiments, the inhibitory polypeptide sequence comprises a cytokine-binding domain. The cytokine-binding domain may be the cytokine-binding domain of a cytokine receptor. The cytokine-binding domain of a cytokine receptor may be provided as an extracellular portion of the cytokine receptor or a portion thereof sufficient to bind the cytokine polypeptide sequence of the linker polypeptide. In some embodiments, the inhibitory polypeptide sequence has at least 80, 85, 90, 95, 97, 98, or 99 percent identity to the sequence of a wild-type cytokine-binding domain of a cytokine receptor, e.g., a wild-type cytokine-binding domain of a human cytokine receptor.

The cytokine-binding domain may be a fibronectin cytokine-binding domain. In some embodiments, the inhibitory polypeptide sequence has at least 80, 85, 90, 95, 97, 98, or 99 percent identity to the sequence of a wild-type fibronectin cytokine-binding domain of a cytokine receptor, e.g., a wild-type human fibronectin cytokine-binding domain.

In some embodiments, the inhibitory polypeptide sequence comprises an amino acid sequence having at least 80, 85, 90, 95, 97, 98, or 99 percent identity to the sequence of any one of SEQ ID NOs: 10-29, 40-51, 747, 748 and 749, 850-856, 939, 940, 941 and 945, 950 and 952, 953, 954 and 955, 956, 957 and 958, 959, 960 and 961, 962, 963 and 964, 965, 966 and 967, 968, 969 and 970, 971, 972 and 973, 974, 975 and 976, 977, 978 and 979, 980, 981 and 982, 983, 984 and 985, 986, 987 and 988, 989, 990, 991 and 992, 999 and 1000, 1001, 1002, 1003 and 1004, 1005, 1006, 1008 and 1010 (where pairs of SEQ ID NOs linked by “and” indicate a VH and VL pair that together can form an inhibitory polypeptide sequence, e.g., as separate chains or as a single chain joined by a linker). In some embodiments, the inhibitory polypeptide sequence comprises an amino acid sequence having at least 80, 85, 90, 95, 97, 98, or 99 percent identity to the sequence of SEQ ID NO: 1011 or 1012. In some embodiments, the inhibitory polypeptide sequence comprises an amino acid sequence having at least 80, 85, 90, 95, 97, 98, or 99 percent identity to the sequence of any one of SEQ ID NOs: 1016-1019. In some embodiments, the inhibitory polypeptide sequence comprises an amino acid sequence having at least 80, 85, 90, 95, 97, 98, or 99 percent identity to the sequence of SEQ ID NO: 1020, 1021, or 1023. In any of the foregoing embodiments, the VH and VL domains may comprise CDRs identical to the CDRs of the referenced SEQ ID NO(s). CDRs may be identified by any appropriate method, such as that of Kabat (as described in Kabat et al., (5^{th} Ed. 1991) Sequences of Proteins of Immunological Interest, available at books.google.co.uk/books?id=3jMvZYW2ZtwC&lpg=PA1137-IA1&pg=PP1#v=onepage&q&f=false) or Chothia (as described in Al-Lazikani et al., (1997) JMB 273, 927-948). In some embodiments, the inhibitory polypeptide sequence comprises VH and VL domains comprising the CDRs of any of SEQ ID NO: 747, 748 and 749, 939, 940, 941 and 945, 950 and 952, 953, 954 and 955, 956, 957 and 958, 959, 960 and 961, 962, 963 and 964, 965, 966 and 967, 968, 969 and 970, 971, 972 and 973, 974, 975 and 976, 977, 978 and 979, 980, 981 and 982, 983, 984 and 985, 986, 987 and 988, 989, 990, 991 and 992, 999 and 1000, 1001, 1002, 1003 and 1004, 1005, 1006, 1008 and 1010. In some embodiments, the inhibitory polypeptide sequence comprises the sequence of any of SEQ ID NO: 747, 748 and 749, 939, 940, 941 and 945, 950 and 952, 953, 954 and 955, 956, 957 and 958, 959, 960 and 961, 962, 963 and 964, 965, 966 and 967, 968, 969 and 970, 971, 972 and 973, 974, 975 and 976, 977, 978 and 979, 980, 981 and 982, 983, 984 and 985, 986, 987 and 988, 989, 990, 991 and 992, 999 and 1000, 1001, 1002, 1003 and 1004, 1005, 1006, 1008 and 1010.

In some embodiments, the inhibitory polypeptide sequence comprises an amino acid sequence having at least 80, 85, 90, 95, 97, 98, or 99 percent identity to the sequence of any one of SEQ ID NOs: 850-856 and 863-870. In any of the foregoing embodiments, the VHH domain may comprise CDRs identical to the CDRs of any one of SEQ ID NOs: 850-856 and 863-870. In some embodiments, the inhibitory polypeptide sequence comprises a VHH comprising the CDRs of any one of SEQ ID NOs: 850-856 and 863-870. In some embodiments, the inhibitory polypeptide sequence comprises the sequence of any one of SEQ ID NOs: 850-856 and 863-870.

In some embodiments, the cytokine-binding domain may be an immunoglobulin cytokine-binding domain. In some embodiments, the immunoglobulin cytokine-binding domain comprises a VH region and a VL region that bind the cytokine. In some embodiments, the immunoglobulin cytokine-binding domain may be an Fv, scFv, Fab, VHH, or other immunoglobulin sequence having antigen-binding activity for the cytokine polypeptide sequence. A VHH antibody (or nanobody) is an antigen binding fragment of a heavy chain only antibody.

Additional examples of inhibitory polypeptide sequences that may be provided to inhibit the cytokine polypeptide sequence of the linker polypeptide are anticalins, affilins, affibody molecules, affimers, affitins, alphabodies, avimers, DARPins, fynomers, kunitz domain peptides, monobodies, and binding domains based on other engineered scaffolds such as SpA, GroEL, lipocallin and CTLA4 scaffolds.

In linker polypeptides comprising an IL-2 polypeptide sequence, the inhibitory polypeptide sequence may be an IL-2 inhibitory polypeptide sequence of any of the types described above. In some embodiments, the IL-2 inhibitory polypeptide sequence is an immunoglobulin IL-2 inhibitory polypeptide sequence.

In some embodiments, the IL-2 inhibitory polypeptide sequence comprises an anti-IL-2 antibody or a functional fragment thereof. In some embodiments, the inhibitory polypeptide sequence comprises an IL-2-binding immunoglobulin domain. In some embodiments, the IL-2-binding immunoglobulin domain is a human IL-2-binding immunoglobulin domain.

In some embodiments, the IL-2-binding immunoglobulin domain is an scFv. In some embodiments, the IL-2-binding immunoglobulin domain comprises a set of six anti-IL-2 hypervariable regions (HVRs) set forth in Table 1 (e.g., SEQ ID NOs: 34-39 or 750-755). In some embodiments, the IL-2-binding immunoglobulin domain comprises a set of anti-IL-2 VH and VL regions comprising sequences having at least 80, 85, 90, 95, 97, 98, or 99 percent identity to the sequence of a set of anti-IL-2 VH and VL regions comprising sequences set forth in Table 1, either as individual sequences or as part of an scFv. In some embodiments, an IL-2-binding immunoglobulin domain comprises a set of anti-IL-2 VH and VL regions having the sequence of a set of anti-IL-2 VH and VL sequences set forth in Table 1, either as individual sequences or as part of an scFv.

Exemplary IL-2 inhibitory polypeptide sequences include SEQ ID NOs: 10-31, 40-51, 747, and 850-856, and a combination of SEQ ID NOs: 32 and 33 or a combination of SEQ ID NOs: 748 and 749. In some embodiments, the IL-2 inhibitory polypeptide sequence comprises an IL-2-binding immunoglobulin domain, which comprises a VH region comprising an amino acid sequence having at least 80, 85, 90, 95, 97, 98, or 99 percent identity to the sequence of SEQ ID NO: 33 and a VL region comprising an amino acid sequence having at least 80, 85, 90, 95, 97, 98, or 99 percent identity to the sequence of SEQ ID NO: 32. In some embodiments, the IL-2-binding immunoglobulin domain comprises a VH region comprising the sequence of SEQ ID NO: 33 and a VL region comprising the sequence of SEQ ID NO: 32.

In some embodiments, the IL-2-binding immunoglobulin domain comprises a VH region comprising hypervariable regions HVR-1, HVR-2, and HVR-3 having the sequences of SEQ ID NOs: 37, 38, and 39, respectively, and a VL region comprising HVR-1, HVR-2, and HVR-3 having the sequences of SEQ ID NOs: 34, 35, and 36, respectively. In some embodiments, the IL-2-binding immunoglobulin domain comprises an amino acid sequence having at least 80, 85, 90, 95, 97, 98, or 99 percent identity to the sequence of SEQ ID NO: 30 or 31. In some embodiments, the IL-2-binding immunoglobulin domain comprises the sequence of SEQ ID NO: 30 or 31.

In some embodiments, the inhibitory polypeptide sequence comprises an IL-2 binding domain of an IL-2 receptor (IL-2R). In some embodiments, the IL-2R is a human IL-2R.

In linker polypeptides comprising an IL-10 polypeptide sequence, the inhibitory polypeptide sequence may be an IL-10 inhibitory polypeptide sequence of any of the types described above. In some embodiments, the IL-10 inhibitory polypeptide sequence is an immunoglobulin IL-10 inhibitory polypeptide sequence.

In some embodiments, the IL-10 inhibitory polypeptide sequence comprises an anti-IL-10 antibody or a functional fragment thereof. In some embodiments, the inhibitory polypeptide sequence comprises an IL-10-binding immunoglobulin domain. In some embodiments, the IL-10-binding immunoglobulin domain is a human IL-10-binding immunoglobulin domain.

In some embodiments, the IL-10-binding immunoglobulin domain is an scFv. In some embodiments, the IL-10-binding immunoglobulin domain comprises a set of six anti-IL-10 hypervariable regions (HVRs) set forth in Table 1 (e.g., SEQ ID NOs: 942-944 and 946-948). In some embodiments, the IL-10-binding immunoglobulin domain comprises a set of anti-IL-10 VH and VL regions comprising sequences having at least 80, 85, 90, 95, 97, 98, or 99 percent identity to the sequence of a set of anti-IL-10 VH and VL regions comprising sequences set forth in Table 1, either as individual sequences or as part of an scFv. In some embodiments, an IL-10-binding immunoglobulin domain comprises a set of anti-IL-10 VH and VL regions having the sequence of a set of anti-IL-10 VH and VL sequences set forth in Table 1, either as individual sequences or as part of an scFv.

Exemplary IL-10 inhibitory polypeptide sequences include SEQ ID NOs: 939-948, 1011, and 1012. In some embodiments, the IL-10 inhibitory polypeptide sequence comprises an IL-10-binding immunoglobulin domain, which comprises a VH region comprising an amino acid sequence having at least 80, 85, 90, 95, 97, 98, or 99 percent identity to the sequence of SEQ ID NO: 945 and a VL region comprising an amino acid sequence having at least 80, 85, 90, 95, 97, 98, or 99 percent identity to the sequence of SEQ ID NO: 941. In some embodiments, the IL-10-binding immunoglobulin domain comprises a VH region comprising the sequence of SEQ ID NO: 945 and a VL region comprising the sequence of SEQ ID NO: 941.

In some embodiments, the IL-10-binding immunoglobulin domain comprises a VH region comprising hypervariable regions HVR-1, HVR-2, and HVR-3 having the sequences of SEQ ID NOs: 946, 947, and 948, respectively, and a VL region comprising HVR-1, HVR-2, and HVR-3 having the sequences of SEQ ID NOs: 942, 943, and 944, respectively. In some embodiments, the IL-10-binding immunoglobulin domain comprises an amino acid sequence having at least 80, 85, 90, 95, 97, 98, or 99 percent identity to the sequence of SEQ ID NO: 939 or 940. In some embodiments, the IL-10-binding immunoglobulin domain comprises the sequence of SEQ ID NO: 939 or 940.

In some embodiments, the inhibitory polypeptide sequence comprises an IL-10 binding domain of an IL-10 receptor (IL-10R). In some embodiments, the IL-10R is a human IL-10R.

In linker polypeptides comprising an IL-15 polypeptide sequence, the inhibitory polypeptide sequence may be an IL-15 inhibitory polypeptide sequence of any of the types described above. In some embodiments, the IL-15 inhibitory polypeptide sequence is an immunoglobulin IL-15 inhibitory polypeptide sequence.

In some embodiments, the IL-15 inhibitory polypeptide sequence comprises an anti-IL-15 antibody or a functional fragment thereof. In some embodiments, the inhibitory polypeptide sequence comprises an IL-15-binding immunoglobulin domain. In some embodiments, the IL-15-binding immunoglobulin domain is a human IL-15-binding immunoglobulin domain.

In some embodiments, the IL-15-binding immunoglobulin domain is an scFv. In some embodiments, the IL-15-binding immunoglobulin domain comprises a VH region comprising HVR-1, HVR-2, and HVR-3 of a VH region comprising the amino acid sequence of any one of SEQ ID NOs: 950, 955, 957, 960, 963, 966, 969, 972, 975, 978, 981, 985, and 988, and a VL region comprising HVR-1, HVR-2, and HVR-3 of a VL region comprising the amino acid sequence of any one of SEQ ID NOs: 952, 954, 958, 961, 964, 967, 970, 973, 976, 979, 982, 984, and 987. In general, a person skilled in the art can identify the HVRs in VH and VL sequences, e.g., by assigning amino acids to framework and HVR domains within the VH and VL sequences in accordance with the definitions of Kabat et al. in Sequences of Proteins of Immunological Interest, 5^{th} Ed., US Dept. of Health and Human Services, PHS, NIH, NIH Publication no. 91-3242, 1991. Other numbering systems for the amino acids in immunoglobulin chains include IMGT™ (international ImMunoGeneTics information system; Lefranc et al, Dev. Comp. Immunol. 29:185-203; 2005) and AHo (Honegger and Pluckthun, J. Mol. Biol. 309(3):657-670; 2001). In some embodiments, the IL-15-binding immunoglobulin domain comprises a set of anti-IL-15 VH and VL regions comprising sequences having at least 80, 85, 90, 95, 97, 98, or 99 percent identity to the sequence of a set of anti-IL-15 VH and VL regions comprising sequences set forth in Table 1, either as individual sequences or as part of an scFv. In some embodiments, an IL-15-binding immunoglobulin domain comprises a set of anti-IL-15 VH and VL regions having the sequence of a set of anti-IL-15 VH and VL sequences set forth in Table 1, either as individual sequences or as part of an scFv.

Exemplary IL-15 inhibitory polypeptide sequences include SEQ ID NOs: 953, 956, 959, 962, 965, 968, 971, 974, 977, 980, 983, and 986. In some embodiments, the IL-15 inhibitory polypeptide sequence comprises an IL-15-binding immunoglobulin domain, which comprises a VH region comprising an amino acid sequence having at least 80, 85, 90, 95, 97, 98, or 99 percent identity to any one of SEQ ID NOs: 950, 955, 957, 960, 963, 966, 969, 972, 975, 978, 981, 985, and 988 and a VL region comprising an amino acid sequence having at least 80, 85, 90, 95, 97, 98, or 99 percent identity to the any one of SEQ ID NOs: 952, 954, 958, 961, 964, 967, 970, 973, 976, 979, 982, 984, and 987. In some embodiments, the IL-15-binding immunoglobulin domain comprises a VH region comprising the sequence of any one of SEQ ID NOs: 950, 955, 957, 960, 963, 966, 969, 972, 975, 978, 981, 985, and 988 and a VL region comprising the sequence of any one of SEQ ID NOs: 952, 954, 958, 961, 964, 967, 970, 973, 976, 979, 982, 984, and 987.

In some embodiments, the inhibitory polypeptide sequence comprises an IL-15 binding domain of an IL-15 receptor (IL-15R). In some embodiments, the IL-15R is a human IL-15R.

In linker polypeptides comprising an CXCL9 polypeptide sequence, the inhibitory polypeptide sequence may be an CXCL9 inhibitory polypeptide sequence of any of the types described above. In some embodiments, the CXCL9 inhibitory polypeptide sequence is an immunoglobulin CXCL9 inhibitory polypeptide sequence.

In some embodiments, the CXCL9 inhibitory polypeptide sequence comprises an anti-CXCL9 antibody or a functional fragment thereof. In some embodiments, the inhibitory polypeptide sequence comprises an CXCL9-binding immunoglobulin domain. In some embodiments, the CXCL9-binding immunoglobulin domain is a human CXCL9-binding immunoglobulin domain.

Exemplary CXCL9 inhibitory polypeptide sequences include SEQ ID NOs: 1020-1021. In some embodiments, the inhibitory polypeptide sequence comprises an CXCL9 binding domain of an CXCL9 receptor (CXCR3). In some embodiments, the CXCR3 is a human CXCR3.

In linker polypeptides comprising an CXCL10 polypeptide sequence, the inhibitory polypeptide sequence may be an CXCL10 inhibitory polypeptide sequence of any of the types described above. In some embodiments, the CXCL10 inhibitory polypeptide sequence is an immunoglobulin CXCL10 inhibitory polypeptide sequence.

In some embodiments, the CXCL10 inhibitory polypeptide sequence comprises an anti-CXCL10 antibody or a functional fragment thereof. In some embodiments, the inhibitory polypeptide sequence comprises an CXCL10-binding immunoglobulin domain. In some embodiments, the CXCL10-binding immunoglobulin domain is a human CXCL10-binding immunoglobulin domain.

In some embodiments, the CXCL10-binding immunoglobulin domain is an scFv. In some embodiments, the CXCL10-binding immunoglobulin domain comprises a set of six anti-CXCL10 hypervariable regions (HVRs) set forth in Table 1 (e.g., SEQ ID NOs: 993-998). In some embodiments, the CXCL10-binding immunoglobulin domain comprises a set of anti-CXCL10 VH and VL regions comprising sequences having at least 80, 85, 90, 95, 97, 98, or 99 percent identity to the sequence of a set of anti-CXCL10 VH and VL regions comprising sequences set forth in Table 1, either as individual sequences or as part of an scFv. In some embodiments, a CXCL10-binding immunoglobulin domain comprises a set of anti-CXCL10 VH and VL regions having the sequence of a set of anti-CXCL10 VH and VL sequences set forth in Table 1, either as individual sequences or as part of an scFv.

Exemplary CXCL10 inhibitory polypeptide sequences include SEQ ID NOs: 989 and 990. In some embodiments, the CXCL10 inhibitory polypeptide sequence comprises an CXCL10-binding immunoglobulin domain, which comprises a VH region comprising an amino acid sequence having at least 80, 85, 90, 95, 97, 98, or 99 percent identity to the sequence of SEQ ID NO: 991 and a VL region comprising an amino acid sequence having at least 80, 85, 90, 95, 97, 98, or 99 percent identity to the sequence of SEQ ID NO: 992. In some embodiments, the CXCL10-binding immunoglobulin domain comprises a VH region comprising the sequence of SEQ ID NO: 991 and a VL region comprising the sequence of SEQ ID NO: 992.

In some embodiments, the CXCL10-binding immunoglobulin domain comprises a VH region comprising hypervariable regions HVR-1, HVR-2, and HVR-3 having the sequences of SEQ ID NOs: 993, 994, and 995, respectively, and a VL region comprising HVR-1, HVR-2, and HVR-3 having the sequences of SEQ ID NOs: 996, 997, and 998, respectively. In some embodiments, the CXCL10-binding immunoglobulin domain comprises an amino acid sequence having at least 80, 85, 90, 95, 97, 98, or 99 percent identity to the sequence of SEQ ID NO: 989 or 990. In some embodiments, the CXCL10-binding immunoglobulin domain comprises the sequence of SEQ ID NO: 989 or 990.

In some embodiments, the inhibitory polypeptide sequence comprises an CXCL10 binding domain of an CXCL10 receptor (CXCR3). In some embodiments, the CXCR3 is a human CXCR3.

### C. Linker

A variety of linkers may be used in accordance with the present disclosure. In many embodiments, a linker may be used to connect any two domains in a linker polypeptide. In some embodiments, a linker polypeptide comprises one linker. In other embodiments, a linker polypeptide may comprise two or more linkers. In some embodiments, a first linker exists between a pharmacokinetic modulator and a first active domain. In some embodiments, a second linker exists between a receptor-binding domain and an inhibitory polypeptide sequence. In some embodiments, the first linker and/or the second linker comprises a protease-cleavable polypeptide sequence. In some embodiments, after the protease-cleavable polypeptide sequence is cleaved, the first active domain and/or the second active domain is released from the remainder of the linker polypeptide. In some embodiments, the linker polypeptide comprises a plurality of protease-cleavable polypeptide sequences.

In these embodiments, different linkers may be used to provide different release properties for different linked domains. For example, a linker for releasing a target binding domain, such as an immunoglobulin antigen-binding domain, may differ from a linker for releasing a receptor-binding domain, such as a cytokine polypeptide sequence. A linker may comprise any of the exemplary linker sequences disclosed herein, e.g., in Table 1.

### 1. Protease-Cleavable Sequence

The protease-cleavable sequence may comprise a sequence cleavable and/or recognized by various types of proteases, e.g., a metalloprotease, a serine protease, a cysteine protease, an aspartate protease, a threonine protease, a glutamate protease, a gelatinase, an asparagine peptide lyase, a cathepsin, a kallikrein, a plasmin, a collagenase, a hK1, a hK10, a hK15, a stromelysin, a Factor Xa, a chymotrypsin-like protease, a trypsin-like protease, a elastase-like protease, a subtilisin-like protease, an actinidain, a bromelain, a calpain, a caspase, a Mir 1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, an ADAM 10, an ADAM 17, an ADAM 12, an urokinase plasminogen activator (uPA), an enterokinase, a prostate-specific target (PSA, hK3), an interleukin-1b converting enzyme, a thrombin, a FAP (FAP-a), a dipeptidyl peptidase, or dipeptidyl peptidase IV (DPPIV/CD26), a type II transmembrane serine protease (TTSP), a neutrophil elastase, a proteinase 3, a mast cell chymase, a mast cell tryptase, or a dipeptidyl peptidase. In some embodiments, the protease-cleavable sequence comprises a sequence of any one of those in Table 1 (e.g., SEQ ID NOs: 80-94 and 701-742), or a variant having one or two mismatches relative to a sequence of any one of those in Table 1 (e.g., SEQ ID NOs: 80-90 and 701-742). Proteases generally do not require an exact copy of the recognition sequence, and as such, the exemplary sequences may be varied at one or more portions of their amino acid positions. In some embodiments, the protease-cleavable sequence comprises a sequence that matches an MMP consensus sequence, such as any one of SEQ ID NOs: 91-94.

One skilled in the art will be familiar with additional sequences recognized by these types of proteases.

i. Matrix Metalloprotease-Cleavable Sequence

In some embodiments, the protease-cleavable sequence is a matrix metalloprotease (MMP)-cleavable sequence and is recognized by a matrix metalloprotease. Exemplary MMP-cleavable sequences are provided in Table 1. In some embodiments, the MMP-cleavable sequence is cleavable and/or recognized by a plurality of MMPs and/or one or more of MMP-1, MMP-2, MMP-3, MMP-7, MMP-8, MMP-9, MMP-12, MMP-13, and/or MMP-14. In some embodiments, the protease-cleavable polypeptide sequence is cleavable and/or recognized by two, three, four, five, six, or seven of MMP-2, MMP-7, MMP-8, MMP-9, MMP-12, MMP-13, and MMP-14. Table 1, e.g., SEQ ID NOs: 80-90, provides exemplary MMP-cleavable sequences.

In some embodiments, the protease-cleavable polypeptide sequence comprises a sequence of any one of SEQ ID NO: 80-90. In some embodiments, the protease-cleavable polypeptide sequence comprises the sequence of SEQ ID NO: 80 or a variant sequence having one or two mismatches relative thereto. In some embodiments, the protease-cleavable polypeptide sequence comprises the sequence of SEQ ID NO: 81 or a variant sequence having one or two mismatches relative thereto. In some embodiments, the protease-cleavable polypeptide sequence comprises the sequence of SEQ ID NO: 82 or a variant sequence having one or two mismatches relative thereto. In some embodiments, the protease-cleavable polypeptide sequence comprises the sequence of SEQ ID NO: 83 or a variant sequence having one or two mismatches relative thereto. In some embodiments, the protease-cleavable polypeptide sequence comprises the sequence of SEQ ID NO: 84 or a variant sequence having one or two mismatches relative thereto. In some embodiments, the protease-cleavable polypeptide sequence comprises the sequence of SEQ ID NO: 85 or a variant sequence having one or two mismatches relative thereto. In some embodiments, the protease-cleavable polypeptide sequence comprises the sequence of SEQ ID NO: 86 or a variant sequence having one or two mismatches relative thereto. In some embodiments, the protease-cleavable polypeptide sequence comprises the sequence of SEQ ID NO: 87 or a variant sequence having one or two mismatches relative thereto. In some embodiments, the protease-cleavable polypeptide sequence comprises the sequence of SEQ ID NO: 88 or a variant sequence having one or two mismatches relative thereto. In some embodiments, the protease-cleavable polypeptide sequence comprises the sequence of SEQ ID NO: 89 or a variant sequence having one or two mismatches relative thereto. In some embodiments, the protease-cleavable polypeptide sequence comprises the sequence of SEQ ID NO: 90 or a variant sequence having one or two mismatches relative thereto. In some embodiments, the protease-cleavable polypeptide sequence comprises the sequence of SEQ ID NO: 91 or a variant sequence having one or two mismatches relative thereto. In some embodiments, the protease-cleavable polypeptide sequence comprises the sequence of SEQ ID NO: 92 or a variant sequence having one or two mismatches relative thereto. In some embodiments, the protease-cleavable polypeptide sequence comprises the sequence of SEQ ID NO: 93 or a variant sequence having one or two mismatches relative thereto. In some embodiments, the protease-cleavable polypeptide sequence comprises the sequence of SEQ ID NO: 94 or a variant sequence having one or two mismatches relative thereto.

### D. Targeting Sequence

In some embodiments, the linker polypeptide comprises a first targeting sequence and/or a second targeting sequence. In some embodiments, the first targeting sequence and/or the second targeting sequence is between a receptor-binding domain and a protease-cleavable polypeptide sequence or one of a plurality of protease-cleavable polypeptide sequences. In some embodiments, at least one of the first linker and the second linker comprises a targeting sequence, e.g., one of the first targeting sequence and the second targeting sequence, at least one targeting sequence, one of a first plurality of targeting sequences, one of a second plurality of targeting sequences, or one of a plurality of targeting sequences. In some embodiments, the protease-cleavable polypeptide sequence comprises a targeting sequence, e.g., one of the first targeting sequence and the second targeting sequence, the at least one targeting sequence, one of the first plurality of targeting sequences, one of the second plurality of targeting sequences, or one of the plurality of targeting sequences.

In some embodiments, one or each of the first targeting sequence and the second targeting sequence, one or each of the at least one targeting sequence, one or each of the first plurality of targeting sequences, one or each of the second plurality of targeting sequences, or one or each of the plurality of targeting sequences increases a serum half-life of the linker polypeptide. In general, an increase in serum half-life may be relative, e.g., to the serum half-life of a linker polypeptide that lacks one or each of the first targeting sequence and the second targeting sequence, one or each of the at least one targeting sequence, one or each of the first plurality of targeting sequences, one or each of the second plurality of targeting sequences, or one or each of the plurality of targeting sequences. In some embodiments, one or each of the first targeting sequence and the second targeting sequence, one or each of the at least one targeting sequence, one or each of the first plurality of targeting sequences, one or each of the second plurality of targeting sequences, or one or each of the plurality of targeting sequences synergistically increases a serum half-life of the linker polypeptide together with another one of the first targeting sequence and the second targeting sequence, another one of the at least one targeting sequence, another one of the first plurality of targeting sequences, another one of the second plurality of targeting sequences, or another one of the plurality of targeting sequences. In some embodiments, one or each of the first targeting sequence and the second targeting sequence, one or each of the at least one targeting sequence, one or each of the first plurality of targeting sequences, one or each of the second plurality of targeting sequences, or one or each of the plurality of targeting sequences synergistically increases a serum half-life of the linker polypeptide together with the pharmacokinetic modulator. In some embodiments, one or each of the first targeting sequence and the second targeting sequence, one or each of the at least one targeting sequence, one or each of the first plurality of targeting sequences, one or each of the second plurality of targeting sequences, or one or each of the plurality of targeting sequences independently increases a serum half-life of the linker polypeptide.

Serum half-life may be measured, for example, by measuring serum levels of the linker polypeptide over time after administration of the linker polypeptide. In some embodiments, any one of the above targeting sequences may independently increase the serum half-life of the linker polypeptide when the serum half-life is greater than a serum half-life of a linker polypeptide that lacks the one targeting sequence but that is otherwise identical to the linker polypeptide, and when the increase is independent of any other increase derived from another targeting sequence. In some embodiments, any one of the above targeting sequences may synergistically increase the serum half-life of the linker polypeptide together with the other one of the targeting sequences or with the pharmacokinetic modulator when the increase in serum half-life is greater than the sum of the increase derived from the one targeting sequence and the increase derived from the other one of the targeting sequences, or than the sum of the increase derived from the one targeting sequence and the increase derived from the pharmacokinetic modulator.

The targeting sequence may facilitate localization, accumulation, and/or retention of the linker polypeptide and/or the first active domain and/or the second active domain (e.g., after proteolysis of the protease-cleavable sequence) in an area of interest, e.g., a tumor microenvironment (TME). The targeting sequence may be a sequence that binds an extracellular matrix component. Exemplary extracellular matrix components may include, for example, a collagen or denatured collagen (in either case, the collagen may be collagen I, II, III, or IV), poly(I), von Willebrand factor, IgB (CD79b), a heparin, a heparan sulfate, a sulfated glycoprotein, or hyaluronic acid. In some embodiments, the extracellular matrix component is hyaluronic acid, a heparin, a heparan sulfate, or a sulfated glycoprotein.

In some embodiments, the targeting sequence binds a target other than an extracellular matrix component. In some embodiments, the targeting sequence binds one or more of IgB (CD79b), a fibronectin, an integrin, a cadherin, a heparan sulfate proteoglycan, and a syndecan. In some embodiments, the targeting sequence binds at least one integrin, such as one or more of α1β1 integrin, α2β1 integrin, α3β1 integrin, α4β1 integrin, α5β1 integrin, α6β1 integrin, α7β1 integrin, α9β1 integrin, α4β7 integrin, αvβ integrin, αvβ5 integrin, αIIbβ3 integrin, αIIIbβ3 integrin, αMβ2 integrin, or αIIbβ3 integrin. In some embodiments, the targeting sequence binds at least one syndecan, such as one of more of syndecan-1, syndecan-4, and syndecan-2(w). Linker polypeptides comprising such targeting sequences may also comprise an MMP-cleavable linker as set forth elsewhere herein, such as an MMP-cleavable linker comprising any one of SEQ ID NOs: 80-90, or a variant having one or two mismatches relative to the sequence of any one of SEQ ID NOs: 80-90.

In some embodiments, the targeting sequence comprises a sequence set forth in Table 2 (e.g., any one of SEQ ID NOs: 179-665, such as SEQ ID NOs: 179-640), or a variant having one or two mismatches relative to such a sequence.

In some embodiments that include a first targeting sequence and a second targeting sequence, the first targeting sequence is configured to bind to heparin and the second targeting sequence is configured to bind to heparin, wherein the first targeting sequence is configured to bind to collagen IV and the second targeting sequence is configured to bind to heparin, or wherein the first targeting sequence is configured to bind to heparin and the second targeting sequence is configured to bind to collagen IV.

In some embodiments, one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target with an affinity from 0.1 nM to 1 nM, from 1 nM to 10 nM, from 10 nM to 100 nM, from 100 nM to 1 μM, from 1 μM to 10 μM, or from 10 μM to 100 μM. In some embodiments, one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target with an affinity from 0.1 nM to 1 nM. In some embodiments, one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target with an affinity from 1 nM to 10 nM. In some embodiments, one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target with an affinity from 10 nM to 100 nM. In some embodiments, one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target with an affinity from 100 nM to 1 M. In some embodiments, one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target with an affinity from 1 μM to 10 μM. In some embodiments, one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target with an affinity from 10 μM to 100 μM. In some embodiments, the affinity may be a dissociation constant (K_{D}), which may be measured, for example, through surface plasmon resonance (SPR), an enzyme linked immunosorbent assay (ELISA), or polarization-modulated oblique-incidence reflectivity difference (OI-RD).

1. pH-Sensitive Targeting Sequences

In some embodiments, the targeting sequence is configured to bind its target in a pH-sensitive manner. In some embodiments, the targeting sequence has a higher affinity for its target at a relatively acidic pH than at normal physiological pH (about 7.4). The higher affinity may occur at a pH below 7, e.g., in the range of pH 5.5-7, 6-7, or 5.5-6.5, or below pH 6. The presence of histidines in the targeting sequence can confer pH-sensitive binding. Without wishing to be bound by any particular theory, histidines are considered more likely to be protonated at lower pH and can render binding a negatively-charged target more energetically favorable. Accordingly, in some embodiments, a targeting sequence comprises one or more histidines, e.g., 1, 2, 3, 4, 5, 6, 7, 8, 9, or 10 histidines. Including a pH-sensitive targeting sequence can enhance discrimination between tumor versus normal tissue by the linker polypeptide, such that the linker polypeptide is more preferentially retained in the tumor microenvironment compared to normal extracellular matrix. Thus, a pH-sensitive targeting element can further facilitate tumor specific delivery of the linker polypeptide and thereby further reduce or eliminate toxicity that may result from activity of the linker polypeptide in normal extracellular matrix.

Binding a target in a pH-sensitive manner can be useful where it is desired to localize or retain a linker polypeptide and/or the cytokine polypeptide sequence thereof in an area with a pH different from normal physiological pH. For example, the tumor microenvironment may be more acidic than the blood and/or healthy tissue. As such, binding to a target in a pH-sensitive manner may improve the retention of the linker polypeptide and/or the cytokine polypeptide sequence thereof in the area of interest, which can facilitate lower doses than would otherwise be needed and/or reduce systemic exposure and/or adverse effects.

In some embodiments, the targeting sequence is configured to bind any target described herein in a pH-sensitive manner. In particular embodiments, the target is an extracellular matrix component, IgB (CD79b), an integrin, a cadherin, a heparan sulfate proteoglycan, a syndecan, or a fibronectin. In some embodiments, the extracellular matrix component is hyaluronic acid, heparin, heparan sulfate, or a sulfated glycoprotein. In another particular embodiment, the target is a fibronectin.

Exemplary targeting sequences for conferring target binding in a pH-sensitive manner are provided in Table 2 (e.g., SEQ ID NOs: 641-663). In some embodiments, the targeting sequence comprises the sequence of any one of SEQ ID NOs: 641-663, or a variant having one or two mismatches relative to the sequence of any one of SEQ ID NOs: 641-663.

In some embodiments, the linker polypeptide comprises a targeting sequence is adjacent to a protease cleavable sequence. The targeting sequence and protease cleavable sequence may be any of those described herein. Exemplary combinations of a targeting sequence and a protease cleavable sequence are SEQ ID NOs: 667-673.

### E. Pharmacokinetic Modulators

In some embodiments, the linker polypeptide comprises a pharmacokinetic modulator. The pharmacokinetic modulator may be covalently or noncovalently associated with the linker polypeptide. The pharmacokinetic modulator can extend the half-life of the linker polypeptide, e.g., so that fewer doses are necessary and less of the linker polypeptide needs to be administered over time to achieve a desired result. Various forms of pharmacokinetic modulator are known in the art and may be used in linker polypeptides of this disclosure. In some embodiments, the pharmacokinetic modulator comprises a polypeptide (see examples below). In some embodiments, the pharmacokinetic modulator comprises a non-polypeptide moiety (e.g., polyethylene glycol, a polysaccharide, or hyaluronic acid). A non-polypeptide moiety can be associated with the linker polypeptide using known approaches, e.g., conjugation to the linker polypeptide; for example, a reactive amino acid residue can be used or added to the linker polypeptide to facilitate conjugation.

In some embodiments, the pharmacokinetic modulator alters the size, shape, and/or charge of the linker polypeptide, e.g., in a manner that reduces clearance. For example, a pharmacokinetic modulator with a negative charge may inhibit renal clearance. In some embodiments, the pharmacokinetic modulator increases the hydrodynamic volume of the linker polypeptide. In some embodiments, the pharmacokinetic modulator reduces renal clearance, e.g., by increasing the hydrodynamic volume of the linker polypeptide.

In some embodiments, the linker polypeptide comprising the pharmacokinetic modulator (e.g., any of the pharmacokinetic modulators described herein) has a molecular weight of at least 70 kDa, e.g., at least 75 or 80 kDa.

For further discussion of various approaches for providing a pharmacokinetic modulator, see, e.g., Strohl, BioDrugs 29:215-19 (2015) and Podust et al., J. Controlled Release 240:52-66 (2016).

### 1. Polypeptide Pharmacokinetic Modulators

In some embodiments, the pharmacokinetic modulator comprises a polypeptide, e.g., an immunoglobulin sequence (see exemplary embodiments below), an albumin, a CTP (a negatively-charged carboxy-terminal peptide of the chorionic gonadotropin 3-chain that undergoes sialylation in vivo and in appropriate host cells), an inert polypeptide (e.g., an unstructured polypeptide such as an XTEN, a polypeptide comprising the residues Ala, Glu, Gly, Pro, Ser, and Thr), a transferrin, a homo-amino-acid polypeptide, or an elastin-like polypeptide.

Exemplary polypeptide sequences suitable for use as a pharmacokinetic modulator are provided in Table 1 (e.g., any one of SEQ ID NOs: 70-74). In some embodiments, the pharmacokinetic modulator has at least 80, 85, 90, 95, 97, 98, or 99 percent identity to the sequence of a pharmacokinetic modulator in Table 1 (e.g., any one of SEQ ID NOs: 70-74).

In any embodiment where the pharmacokinetic modulator comprises a polypeptide sequence from an organism, the polypeptide sequence may be a human polypeptide sequence.

### 2. Immunoglobulin Pharmacokinetic Modulators

In some embodiments, the pharmacokinetic modulator comprises an immunoglobulin sequence, e.g., at least a portion of one or more immunoglobulin constant domains. In some embodiments, the pharmacokinetic modulator comprises an immunoglobulin constant domain. In some embodiments, the pharmacokinetic modulator comprises at least a portion of an immunoglobulin Fc region. In some embodiments, the pharmacokinetic modulator comprises an immunoglobulin Fc region.

The immunoglobulin sequence (e.g., at least a portion of one or more immunoglobulin constant domains or Fc region) may be a human immunoglobulin sequence. The immunoglobulin sequence (e.g., at least a portion of one or more immunoglobulin constant domains or Fc region) may have has at least 80, 85, 90, 95, 97, 98, or 99 percent identity to the sequence of a wild-type immunoglobulin sequence (e.g., at least a portion of one or more immunoglobulin constant domains or Fc region), such as a wild-type human immunoglobulin sequence. In any of such embodiments, the immunoglobulin sequence may be an IgG sequence, such as at least a portion of one or more immunoglobulin constant domains or Fc region thereof (e.g., IgG1, IgG2, IgG3, or IgG4, such as at least a portion of one or more immunoglobulin constant domains or Fc region of any of these isotypes). Exemplary immunoglobulin pharmacokinetic modulator sequences include SEQ ID NOs: 70-74, 857, 858, 861, and 862 and the combination of SEQ ID NOs: 756 and 757; 75 and 77; 75 and 78; 76 and 77; 76 and 78; and 859 and 860.

In some embodiments, immunoglobulin pharmacokinetic modulator sequences (such as an Fc region) may perform certain functions and effects by interacting with certain targets, as described in Table 3 below.

### F. Growth Factor-Binding Polypeptide Sequence and Growth Factor Receptor-Binding Polypeptide Sequence

In some embodiments, the linker polypeptide comprises a growth factor-binding polypeptide sequence or a growth factor receptor-binding polypeptide sequence. Such a sequence can serve as an active domain.

In some embodiments, the growth factor-binding polypeptide sequence comprises a TGF-βR extracellular domain sequence. In some embodiments, the TGF-βR extracellular domain sequence comprises an amino acid sequence having at least 80, 85, 90, 95, 97, 98, or 99 percent identity to the sequence of SEQ ID NO: 1022 or 1023.

In some embodiments, the growth factor-binding polypeptide sequence comprises a growth factor-binding immunoglobulin domain. In some embodiments, the growth factor-binding immunoglobulin domain is configured to bind to a TGF-β. In some embodiments, the growth factor-binding immunoglobulin domain comprises a VH region comprising HVR-1, HVR-2, and HVR-3 of a VH region comprising the amino acid sequence of SEQ ID NO: 1008, and a VL region comprising HVR-1, HVR-2, and HVR-3 of a VL region comprising the amino acid sequence of SEQ ID NO: 1010. In general, a person skilled in the art can identify the HVRs in VH and VL sequences, e.g., by assigning amino acids to framework and HVR domains within the VH and VL sequences in accordance with the definitions of Kabat et al. in Sequences of Proteins of Immunological Interest, 5^{th} Ed., US Dept. of Health and Human Services, PHS, NIH, NIH Publication no. 91-3242, 1991. Other numbering systems for the amino acids in immunoglobulin chains include IMGT™ (international ImMunoGeneTics information system; Lefranc et al, Dev. Comp. Immunol. 29:185-203; 2005) and AHo (Honegger and Pluckthun, J. Mol. Biol. 309(3):657-670; 2001). In some embodiments, the growth factor-binding immunoglobulin domain comprises a VH region comprising the amino acid sequence of SEQ ID NO: 1008; and a VL region comprising the amino acid sequence of SEQ ID NO: 1010. In some embodiments, the growth factor-binding immunoglobulin domain comprises a sequence that has at least 80, 85, 90, 95, 97, 98, or 99 percent identity to the sequence of SEQ ID NO: 1007 or 1009. In some embodiments, the growth factor receptor-binding polypeptide sequence comprises a TGF-0 sequence. In some embodiments, the TGF-0 sequence comprises an amino acid sequence having at least 80, 85, 90, 95, 97, 98, or 99 percent identity to the sequence of any one of SEQ ID NOs. 904-906.

In some embodiments, the growth factor receptor-binding polypeptide sequence comprises a growth factor receptor-binding immunoglobulin domain. In some embodiments, the growth factor receptor-binding immunoglobulin domain is configured to bind to a TGF-βR extracellular domain sequence. In some embodiments, the growth factor receptor-binding immunoglobulin domain comprises a VH region comprising HVR-1, HVR-2, and HVR-3 of a VH region comprising the amino acid sequence of SEQ ID NO: 999 or 1003, and a VL region comprising HVR-1, HVR-2, and HVR-3 of a VL region comprising the amino acid sequence of SEQ ID NO: 1000 or 1004. In some embodiments, the growth factor receptor-binding immunoglobulin domain comprises a VH region comprising the amino acid sequence of SEQ ID NO: 999 or 1003; and a VL region comprising the amino acid sequence of SEQ ID NO: 1000 or 1004. In some embodiments, the growth factor receptor-binding immunoglobulin domain comprises a sequence that has at least 80, 85, 90, 95, 97, 98, or 99 percent identity to the sequence of any one of SEQ ID NOs: 1001, 1002, 1005, and 1006.

TABLE 3
Pharmacokinetic Modulator Functions, Effects, and Targets

| Function Mode | Target | Effects |
|---|---|---|
| Antibody Dependent | FcgR binding site | Kill Fab-bound cells |
| Cellular Cytotoxcity | | |
| (ADCC) | | |
| Antibody Dependent | FcgR binding site | Kill Fab-bound cells |
| Cellular Phagocytosis | | |
| (ADCP) | | |
| Complement Dependent | C1q binding site | Kill Fab-bound cells |
| Cytotoxicity (CDC) | | |
| Antibody Drug Conjugate | Fab | Kill Fab-bound cells |
| (ADC) | | |
| Fc-Recycle | FcRn binding site | Half-life extension |

### A. Blocker

In some embodiments, the linker polypeptide may comprise a blocker. In some embodiments, the blocker may be conjugated to one of or each of the first active domain and the second active domain. In some embodiments, the blocker is conjugated to one of or each of the first active domain and the second active domain via a protease-cleavable polypeptide sequence.

The blocker may obstruct an immunoglobulin antigen-binding domain from binding to an antigen (e.g., a growth factor or growth factor receptor). In some embodiments, the blocker is linked to the immunoglobulin antigen-binding domain through the N-terminus of a heavy or light chain of the immunoglobulin antigen-binding domain.

In some embodiments, the blocker comprises albumin. In some embodiments, the blocker comprises serium albumin. In some embodiments, the blocker comprises human serum albumin (HAS) (e.g., SEQ ID NO: 72) or a fragment thereof.

### B. Chemotherapy Drug

In some embodiments, the linker polypeptide may comprise a chemotherapy drug or a plurality of chemotherapy drugs. The drug may, for example, be conjugated to different elements of the linker polypeptide. In some embodiments, a chemotherapy drug is conjugated to a pharmacokinetic modulator of the linker polypeptide.

In some embodiments, the chemotherapy drug is selected from altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, epirubicin, idarubicin, valrubicin, bleomycin, dactinomycin, mitomycin-c, mitoxantrone, irinotecan, topotecan, etoposide, mitoxantrone, teniposide, cabazitaxel, docetaxel, paclitaxel, vinblastine, vincristine, vinorelbine, prednisone, methylprednisolone, dexamethasone, retinoic acid, arsenic trioxide, asparaginase, eribulin, hydroxyurea, ixabepilone, mitotane, omacetaxine, pegaspargase, procarbazine, romidepsin, and vorinostat.

### III. Arrangement of Components and Release Thereof

The recitation of components of a linker polypeptide herein does not imply any particular order beyond what is explicitly stated (for example, it may be explicitly stated that a protease-cleavable sequence is between the cytokine polypeptide sequence and the inhibitory polypeptide sequence). The components of the linker polypeptide may be arranged in various ways to provide properties suitable for a particular use. The components of the linker polypeptide may be all in one polypeptide chain or they may be in a plurality of polypeptide chains bridged by covalent bonds, such as disulfide bonds.

For example, in some embodiments, where a pharmacokinetic modulator comprises an Fc, one or more components (e.g., chemotherapy drugs) may be bound to one chain while one or more other components may be bound to the other chain. The Fc may be a heterodimeric Fc, such as a knob-into-hole Fc (in which one chain of the Fc comprises knob mutations and the other chain of the Fc comprises hole mutations). For an exemplary general discussion of knob and hole mutations, see, e.g., Xu et al., mAbs 7:1, 231-242 (2015). Exemplary knob mutations (e.g., for a human IgG1 Fc) are K360E/K409W. Exemplary hole mutations (e.g., for a human IgG1 Fc) are Q347R/D399V/F405T. See SEQ ID NOs: 756 and 757.

In some embodiments, some or all of the one or more protease-cleavable polypeptide sequences may be C-terminal to a VH region, C-terminal to at least a portion of a CH1 domain, between a CH1 domain and a CH2 domain, N-terminal to at least a portion of a CH2 domain, N-terminal to a disulfide bond between heavy chains, N-terminal to a disulfide bond within a CH2 domain, or N-terminal to a hinge region, or is within a hinge region. In some embodiments, some or all of the one or more protease-cleavable polypeptide sequences may be between the pharmacokinetic modulator and the second active domain, and/or between the blocker and one or each of the first active domain and the second active domain.

In some embodiments, a targeting sequence may be between the receptor-binding domain and the one or more protease-cleavable polypeptide sequences. In some embodiments, at least one of the first linker and the second linker comprises a targeting sequence, and/or a protease-cleavable polypeptide sequence comprises a targeting sequence.

In some embodiments, a targeting sequence may be present on the same side of a protease-cleavable polypeptide sequence as the receptor-binding domain (e.g., cytokine polypeptide sequence), meaning that cleavage of the protease-cleavable polypeptide sequence does not separate the targeting sequence from the receptor-binding domain. Such embodiments can be useful to facilitate localizing or retaining both the linker polypeptide and the released receptor-binding domain in an area of interest, e.g., a tumor microenvironment.

In some embodiments, a targeting sequence may be present on the same side of a protease-cleavable polypeptide sequence as an inhibitory polypeptide sequence, meaning that cleavage of that protease-cleavable polypeptide sequence does not separate the targeting sequence from the cytokine polypeptide sequence. Such embodiments can be useful to provide a gradient of cytokine emanating from an area of interest, or to provide such a gradient more rapidly than would occur if the targeting sequence were on the same side of the protease-cleavable sequence.

In some embodiments, the first active domain is proximal to the first targeting sequence relative to the second targeting sequence. In other embodiments, the second active domain is proximal to the first targeting sequence relative to the second targeting sequence. In some embodiments, the linker polypeptide comprises sequentially, from the N-terminus to the C-terminus or from the C-terminus to the N-terminus, the first active domain, the first targeting sequence, the first linker, the second targeting sequence, and the additional domain.

In some embodiments, the protease-cleavable polypeptide sequence is C-terminal to the first targeting sequence and to the second targeting sequence. In some embodiments, the protease-cleavable polypeptide sequence is N-terminal to the first targeting sequence and to the second targeting sequence. In some embodiments, the protease-cleavable polypeptide sequence is C-terminal to the first plurality of targeting sequences and is N-terminal to the second plurality of targeting sequences. In some embodiments, the protease-cleavable polypeptide sequence is C-terminal to the plurality of targeting sequences and is N-terminal to at least one targeting sequence. In some embodiments, the protease-cleavable polypeptide sequence is N-terminal to the plurality of targeting sequences and is C-terminal to at least one targeting sequence. In some embodiments, the protease-cleavable polypeptide sequence is C-terminal to the first targeting sequence and to the second targeting sequence and is not N-terminal to a targeting sequence. In some embodiments, the protease-cleavable polypeptide sequence is N-terminal to the first targeting sequence and to the second targeting sequence and is not C-terminal to a targeting sequence.

In some embodiments, the linker polypeptide comprises a first active domain, a second active domain, a pharmacokinetic modulator, and a first linker between the pharmacokinetic modulator and the first active domain. In some embodiments, the first linker comprises a protease-cleavable polypeptide sequence and optionally a targeting sequence. In certain embodiments, the active domains comprise immunoglobulin antigen-binding domains. In certain embodiments, the target binding domain may comprise a heavy chain and a light chain or only a heavy chain. In some embodiments, the linker polypeptide comprises a chemotherapy drug.

In some embodiments, the first active domain is released from the remainder of the linker polypeptide after the one or more protease-cleavable polypeptide sequences are cleaved. In some embodiments, the linker polypeptide further comprises a blocker conjugated, via a protease-cleavable polypeptide sequence, to one or each of the first active domain and the second active domain. In some embodiments, the protease-cleavable polypeptide sequence connecting the first active domain to the remainder of the linker polypeptide and the protease-cleavable polypeptide sequences connecting the blockers to the active domains may be cleaved together (e.g., by the same protease). In some embodiments, the protease-cleavable polypeptide sequence connecting the first active domain to the remainder of the linker polypeptide and the protease-cleavable polypeptide sequences connecting the blockers to the active domains may be cleaved separately (e.g., by different proteases).

In some embodiments, the linker polypeptide comprises a first active domain, a second active domain, a pharmacokinetic modulator, and a first linker between the pharmacokinetic modulator and the first active domain, the first linker comprising a protease-cleavable polypeptide sequence and optionally a targeting sequence. In certain embodiments, the first active domain comprises a receptor-binding domain, and the second active domain comprises an immunoglobulin antigen-binding domain, which may comprise a cytokine polypeptide sequence. In some embodiments, the linker polypeptide comprises an inhibitory polypeptide sequence capable of blocking an activity of the receptor-binding domain, and a second linker between the receptor-binding domain and the inhibitory polypeptide sequence, the second linker comprising a protease-cleavable polypeptide sequence.

In some embodiments, the first active domain is released from the remainder of the linker polypeptide after the one or more protease-cleavable polypeptide sequences are cleaved. In some embodiments, the first active domain comprises a receptor-binding domain, which may comprise a cytokine polypeptide sequence, and the second active domain comprises an immunoglobulin antigen-binding domain. In some embodiments, the linker polypeptide further comprises an inhibitory polypeptide sequence capable of blocking an activity of the receptor-binding domain, and a second linker between the receptor-binding domain and the inhibitory polypeptide sequence, the second linker comprising a protease-cleavable polypeptide sequence. In some embodiments, the protease-cleavable polypeptide sequences of the first linker and the second linker may be cleaved together (e.g., by the same protease). In some embodiments, the protease-cleavable polypeptide sequences of the first linker and the second linker may be cleaved separately (e.g., by different proteases).

In some embodiments, e.g., any of those in which first and second polypeptide chains comprising first and second domains of a pharmacokinetic modulator, respectively, are present, the inhibitory polypeptide sequence is C-terminal to the second domain of the pharmacokinetic modulator, or the inhibitory polypeptide sequence is N-terminal to the second domain of the pharmacokinetic modulator. A targeting sequence may be between the protease-cleavable polypeptide sequence and the first domain of the pharmacokinetic modulator, between the protease-cleavable polypeptide sequence and the first active domain, C-terminal to the first active domain, N-terminal to the first active domain, C-terminal to the inhibitory polypeptide sequence, N-terminal to the inhibitory polypeptide sequence, or between the inhibitory polypeptide sequence and the second domain of the pharmacokinetic modulator.

In some embodiments, e.g., any of those in which first and second polypeptide chains comprising first and second domains of a pharmacokinetic modulator, respectively, are present, the linker polypeptide may comprise first and second targeting sequences. In some such embodiments, the first targeting sequence is part of the first polypeptide chain and the second targeting sequence is part of the second polypeptide chain. In some such embodiments, the first targeting sequence is C-terminal to the first active domain and the second targeting sequence is C-terminal to the inhibitory polypeptide sequence.

In some embodiments, e.g., any of those in which first and second polypeptide chains comprising first and second domains of a pharmacokinetic modulator, respectively, are present, the linker polypeptide further comprises a second active domain, optionally wherein the second active domain is part of the second polypeptide chain, and/or the linker polypeptide comprises a first inhibitory polypeptide sequence and the linker polypeptide further comprises a second inhibitory polypeptide sequence. In some embodiments, the second inhibitory polypeptide sequence is part of the second polypeptide chain. In some embodiments, the second inhibitory polypeptide sequence is C-terminal to the first inhibitory polypeptide sequence. The first and/or second inhibitory polypeptide sequences may be immunoglobulin inhibitory polypeptide sequences, such as a VHH.

In some embodiments, e.g., any of those in which first and second polypeptide chains comprising first and second domains of a pharmacokinetic modulator, respectively, are present, the pharmacokinetic modulator comprises a heterodimeric Fc or heterodimeric CH3 domains. The heterodimeric Fc or heterodimeric CH3 domains may be in separate polypeptide chains. In some embodiments, the heterodimeric Fc or heterodimeric CH3 domains comprise a knob CH3 domain and a hole CH3 domain.

In some embodiments, the linker polypeptide comprises the polypeptide sequence of any one of SEQ ID NOs: 800-848 and 1024-1041. In some embodiments, the linker polypeptide comprises the polypeptide sequence of any one of SEQ ID NOs: 1042-1137.

### IV. Pharmaceutical Formulations or Compositions

Pharmaceutical formulations or compositions of a linker polypeptide as described herein may be prepared by mixing such linker polypeptide having the desired degree of purity with one or more optional pharmaceutically acceptable carriers (Remington's Pharmaceutical Sciences 16th edition, Osol, A. Ed. (1980)), in the form of lyophilized formulations or compositions, or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

The formulations or compositions to be used for in vivo administration are generally sterile. Sterility may be readily accomplished, e.g., by filtration through sterile filtration membranes.

### V. Uses

In some embodiments, any one or more of the linker polypeptides, compositions, or pharmaceutical formulations described herein is for use in therapy, such as in preparing a medicament for treating or preventing a disease or disorder in a subject, such as cancer. In some embodiments, any one or more of the linker polypeptides, compositions, or pharmaceutical formulations described herein is for use in a method of treating a cancer, comprising, for example, administering the linker polypeptide or pharmaceutical composition to a subject in need thereof

In some embodiments, a method of treating or preventing a disease or disorder in subject is provided, comprising administering to a subject any of the linker polypeptides or pharmaceutical compositions described herein. In some embodiments, the disease or disorder is a cancer, e.g., a solid tumor. In some embodiments, the cancer is a melanoma, a colorectal cancer, a breast cancer, a pancreatic cancer, a lung cancer, a prostate cancer, an ovarian cancer, a cervical cancer, a gastric or gastrointestinal cancer, a lymphoma, a colon or colorectal cancer, an endometrial cancer, a thyroid cancer, or a bladder cancer. The cancer (e.g., any of the foregoing cancers) may have one or more of the following features: being PD-L1-positive; being metastatic; being unresectable; being mismatch repair defective (MMRd); and/or being microsatellite-instability high (MSI-H). In some embodiments, the cancer is a TGFβR-expressing cancer. In some embodiments, the cancer is a TGFβ-expressing cancer. In some embodiments, the cancer is a TGFβ-dependent cancer. A cancer is considered dependent on a growth factor such as TGFβ if cells of the cancer grow significantly more slowly in the absence of the growth factor than in its presence.

In some embodiments, a method of boosting T regulatory cells and/or reducing inflammation or autoimmune activity is provided comprising administering a linker polypeptide to an area of interest, e.g., an area of inflammation. The linker polypeptide for use in such methods may comprise an IL-2 polypeptide sequence. In some embodiments, a method of treating an autoimmune and/or inflammatory disease is provided, comprising administering a linker polypeptide to an area of interest, e.g., an area of inflammation or autoimmune activity. The linker polypeptide for use in such methods may comprise an IL-2 polypeptide sequence. These methods take advantage of the ability of certain cytokines at relatively low levels to stimulate T regulatory cells, which can exert anti-inflammatory effects and reduce or suppress autoimmune activity.

The linker polypeptides in any of the foregoing methods and uses may be delivered to a subject using any suitable route of administration. In some embodiments, the linker polypeptide is delivered parenterally. In some embodiments, the linker polypeptide is delivered intravenously.

A linker polypeptide provided herein can be used either alone or in combination with other agents in a therapy. For instance, a linker polypeptide provided herein may be co-administered with at least one additional therapeutic agent.

Such combination therapies noted above encompass combined administration (where two or more therapeutic agents are included in the same or separate formulations), and separate administration, in which case, administration of the linker polypeptide provided herein can occur prior to, simultaneously, and/or following, administration of the additional therapeutic agent and/or adjuvant.

Linker polypeptides would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. In some embodiments, the linker polypeptide is optionally formulated with one or more agents currently used to prevent or treat the disorder in question. The effective amount of such other agents depends on the amount of linker polypeptide present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an linker polypeptide (when used alone or in combination with one or more other additional therapeutic agents) will depend on the type of disease to be treated, the type of linker polypeptide, the severity and course of the disease, whether the linker polypeptide is administered for preventive or therapeutic purposes, previous therapy, the patient's clinical history and response to therapeutic agents (e.g., antibodies, immunoconjugates, cytokines) that share common elements and/or sequences with the linker polypeptide, and the discretion of the attending physician. The linker polypeptide is suitably administered to the patient at one time or over a series of treatments.

### VI. Nucleic Acids, Host Cells, and Production Methods

Linker polypeptides or precursors thereof may be produced using recombinant methods and compositions. In some embodiments, an isolated nucleic acid encoding a linker polypeptide described herein is provided. Such nucleic acid may encode an amino acid sequence comprising active domains (including, for example, an immunoglobulin antigen-binding domain, a receptor-binding domain, and/or a cytokine polypeptide sequence), a pharmacokinetic modulator, a linker, and an inhibitory polypeptide sequence, and any other polypeptide components of the linker polypeptide that may be present. In a further embodiment, one or more vectors (e.g., expression vectors) comprising such nucleic acid are provided. In a further embodiment, a host cell comprising such nucleic acid is provided. In some such embodiments, a host cell comprises (e.g., has been transformed with) a vector comprising a nucleic acid that encodes a linker polypeptide according to the disclosure. In some embodiments, the host cell is eukaryotic, e.g., a Chinese Hamster Ovary (CHO) cell or lymphoid cell (e.g., Y0, NS0, Sp20 cell). In some embodiments, a method of making a linker polypeptide disclosed herein is provided, wherein the method comprises culturing a host cell comprising a nucleic acid encoding the linker polypeptide, as provided above, under conditions suitable for expression of the linker polypeptide, and optionally recovering the antibody from the host cell (or host cell culture medium).

For recombinant production of a linker polypeptide, nucleic acid encoding the linker polypeptide, e.g., as described above, is prepared and/or isolated (e.g., following construction using synthetic and/or molecular cloning techniques) and inserted into one or more vectors for further cloning and/or expression in a host cell. Such nucleic acid may be readily prepared and/or isolated using known techniques.

Suitable host cells for cloning or expression of linker polypeptide-encoding vectors include prokaryotic or eukaryotic cells described herein. For example, a linker polypeptide may be produced in bacteria, in particular when glycosylation is not needed. For expression of polypeptides in bacteria, see, e.g., U.S. Pat. Nos. 5,648,237, 5,789,199, and 5,840,523. After expression, the linker polypeptide may be isolated from the bacterial cell paste in a soluble fraction and can be further purified.

In addition to prokaryotes, eukaryotic microbes such as filamentous fungi or yeast are suitable cloning or expression hosts for linker polypeptide-encoding vectors, including fungi and yeast strains whose glycosylation pathways have been “humanized,” resulting in the production of polypeptides with a partially or fully human glycosylation pattern. See Gerngross, Nat. Biotech. 22:1409-1414 (2004), and Li et al., Nat. Biotech. 24:210-215 (2006).

Suitable host cells for the expression of linker polypeptides are also derived from multicellular organisms (plants, invertebrates, and vertebrates). Examples of invertebrate cells include insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. “About” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

### EXAMPLES

The following examples are provided to illustrate certain disclosed embodiments and are not to be construed as limiting the scope of this disclosure in any way.

### Example 1: Construction of Mammalian Expression Vectors Encoding Fusion Proteins

Coding sequences for all protein domains including linker sequences were synthesized as an entire gene (Genscript, NJ). All synthetic genes were designed to contain a coding sequence for an N-terminal signal peptide (to facilitate protein secretion), a 5′ Kozak sequence, and unique restriction sites at the 5′ and 3′ ends. These genes were then directionally cloned into the mammalian expression vector pcDNA3.1 (Invitrogen, Carlsbad, CA). Examples of fusion protein constructs are listed in Table 4.

TABLE 4
Linker polypeptide constructs

| Lab ID | Features |
|---|---|
| Construct B - no TME | mIL2-2x(SG4)(SEQ ID NO: 1143)-MMPcs1-2x |
| | (G4S)(SEQ ID NO: 1142)-mIL2Ralpha(1-215)- |
| | mIgG1Fc |
| Construct GGG - no TME | mIL2-2x(SG4)(SEQ ID NO: 1143)-MMPscr-2x |
| | (G4S)(SEQ ID NO: 1142)-mIL2Ralpha(1-215)- |
| | mIgG1Fc |
| Construct AAA- no TME | hIL2(C125S)-2x(SG4)(SEQ ID NO: 1143)-MMPcs1- |
| | 2x(G4S)(SEQ ID NO: 1142)-hIL2Ra(M25I)-GSGGGG |
| | (SEQ ID NO: 1138)-huIgG1Fc(LALA) |
| Construct BBB - no TME | hIL2(C125S)-2x(SG4)(SEQ ID NO: 1143)-MMPscr- |
| | 2x(G4S)(SEQ ID NO: 1142)-hIL2Ra(M25I)-GSGGGG |
| | (SEQ ID NO: 1138)-huIgG1Fc(LALA) |
| Construct Y (heparin) | mIL2(C140S)-VRIQRKKEKMKET(SEQ ID NO: |
| | 1139)-MMPcs1-2x(G4S)(SEQ ID NO: 1142)-mIL2Ra |
| | (1-215)-muIgG1Fc |
| Construct AA (heparin) | mIL2-SGG-FHRRIKA(SEQ ID NO: 1140)-MMPcs1- |
| | 2x(G4S)(SEQ ID NO: 1142)-mIL2Ra(1-215)- |
| | muIgG1Fc |
| Construct BB (heparin) | mIL2-SGG-FHRRIKA(SEQ ID NO: 1140)-MMPscr- |
| | 2x(G4S)(SEQ ID NO: 1142)-mIL2Ra(1-215)- |
| | muIgG1Fc |
| Construct CC (pH heparin) | mIL2-2x(GHHPH)(SEQ ID NO: 1141)-MMPcs1- |
| | 2x(G4S)(SEQ ID NO: 1142)-mIL2Ra(1-215)- |
| | muIgG1Fc |
| Construct DD (pH heparin) | mIL2-2x(GHHPH)(SEQ ID NO: 1141)-MMPscr- |
| | 2x(G4S)(SEQ ID NO: 1142)-mIL2Ra(1-215)- |
| | muIgG1Fc |
| Construct EE (pH fibronectin) | mIL2-SGG-GGWSHW(SEQ ID NO: 653)- |
| | MMPcs1-2x(G4S)(SEQ ID NO: 1142)-mIL2Ra(1-215)- |
| | muIgG1Fc |
| Construct FF (pH fibronectin) | mIL2-SGG-GGWSHW(SEQ ID NO: 653)-MMPscr- |
| | 2x(G4S)(SEQ ID NO: 1142)-mIL2Ra(1-215)- |
| | muIgG1Fc |
| Construct GG (collagen IV) | mIL2-SGG-KLWVLPK(SEQ ID NO: 200)- |
| | MMPcs1-2x(G4S)(SEQ ID NO: 1142)-mIL2Ra(1-215)- |
| | muIgG1Fc |
| Construct HH (collagen IV) | mIL2-SGG-KLWVLPK(SEQ ID NO: 200)- |
| | MMPscr-2x(G4S)(SEQ ID NO: 1142)-mIL2Ra(1-215)- |
| | muIgG1Fc |
| Construct II (collagen I) | mIL2-LHERHLNNN(SEQ ID NO: 665)-MMPcs1- |
| | 2x(G4S)(SEQ ID NO: 1142)-mIL2Ra(1-215)- |
| | muIgG1Fc |
| Construct JJ (collagen I) | mIL2-LHERHLNNN(SEQ ID NO: 665)-MMPscr- |
| | 2x(G4S)(SEQ ID NO: 1142)-mIL2Ra(1-215)- |
| | muIgG1Fc |
| Construct KK (heparin) | mIL2-VRIQRKKEKMKET(SEQ ID NO: 1139)- |
| | MMPscr-2x(G4S)(SEQ ID NO: 1142)-mIL2Ra(1-215)- |
| | muIgG1Fc |
| Construct LL (heparin) | mIL2-2x(SG4)(SEQ ID NO: 1143)-MMPcs1- |
| | FHRRIKAGGS(SEQ ID NO: 1144)-mIL2Ralpha(1- |
| | 215)-muIgG1Fc |
| Construct MM (heparin) | mIL2-2x(SG4)(SEQ ID NO: 1143)-MMPscr- |
| | FHRRIKAGGS(SEQ ID NO: 1144)-mIL2Ralpha(1- |
| | 215)-muIgG1Fc |
| Construct NN (pH heparin) | mIL2-2x(SG4)(SEQ ID NO: 1143)-MMPcs1- |
| | 2x(GHHPH)(SEQ ID NO: 1141)-mIL2Ra(1-215)- |
| | muIgG1Fc |
| Construct OO (pH heparin) | mIL2-2x(SG4)(SEQ ID NO: 1143)-MMPscr- |
| | 2x(GHHPH)(SEQ ID NO: 1141)-mIL2Ra(1-215)- |
| | muIgG1Fc |
| Construct PP (pH fibronectin) | mIL2-2x(SG4)(SEQ ID NO: 1143)-MMPcs1- |
| | GGWSHWGGS(SEQ ID NO: 1145)-mIL2Ralpha(1- |
| | 215)-muIgG1Fc |
| Construct QQ (pH fibronectin) | mIL2-2x(SG4)(SEQ ID NO: 1143)-MMPscr- |
| | GGWSHWGGS(SEQ ID NO: 1145)-mIL2Ralpha(1- |
| | 215)-muIgG1Fc |
| Construct RR (collagen IV) | mIL2-2x(SG4)(SEQ ID NO: 1143)-MMPcs1- |
| | KLWVLPKGGS(SEQ ID NO: 1146)-mIL2Ralpha(1- |
| | 215)-muIgG1Fc |
| Construct SS (collagen IV) | mIL2-2x(SG4)(SEQ ID NO: 1143)-MMPscr- |
| | KLWVLPKGGS(SEQ ID NO: 1146)-mIL2Ralpha(1- |
| | 215)-muIgG1Fc |
| Construct TT (collagen I) | mIL2-2x(SG4)(SEQ ID NO: 1143)-MMPcs1- |
| | LHERHLNNNG(SEQ ID NO: 1147)-mIL2Ralpha(1- |
| | 215)-muIgG1Fc |
| Construct UU (collagen I) | mIL2-2x(SG4)(SEQ ID NO: 1143)-MMPscr- |
| | LHERHLNNNG(SEQ ID NO: 1147)-mIL2Ralpha(1- |
| | 215)-muIgG1Fc |
| Construct VV (pH heparin) | mIL2-SGGGGGHHPH(SEQ ID NO: 1148)- |
| | MMPcs1-2x(G4S)(SEQ ID NO: 1142)-mIL2Ra- |
| | muIgG1Fc |
| Construct WW (pH heparin) | mIL2-GHHPHSGGGG(SEQ ID NO: 1149)- |
| | MMPcs1-2x(G4S)(SEQ ID NO: 1142)-mIL2Ra- |
| | muIgG1Fc |
| Construct XX (pH heparin) | mIL2-2x(SG4)(SEQ ID NO: 1143)-MMPcs1- |
| | GHHPHGGGGS(SEQ ID NO: 1150)-mIL2Ra- |
| | muIgG1Fc |
| Construct YY (pH heparin) | mIL2-2x(SG4)(SEQ ID NO: 1143)-MMPcs1- |
| | 2x(G4S)(SEQ ID NO: 1142)-mIL2Ra-muIgG1Fc- |
| | 2x(GHHPH)(SEQ ID NO: 1141) |
| Construct ZZ (pH heparin) | mIL2-2x(SG4)(SEQ ID NO: 1143)-MMPcs1- |
| | 2x(G4S)(SEQ ID NO: 1142)-mIL2Ra-muIgG1Fc- |
| | (GHHPH)(SEQ ID NO: 1141) |
| Construct UUU (hep) | mIL2-2x(SG4)(SEQ ID NO: 1143)-MMPcs1- |
| | VRIQRKKEKMKETGS(SEQ ID NO: 1151)-mIL2Ra- |
| | muIgG1Fc |
| Construct HHH (hep) | mIL2-2x(SG4)(SEQ ID NO: 1143)-MMPcs1- |
| | 2x(G4S)(SEQ ID NO: 1142)-mIL2Ra-muIgG1Fc- |
| | GGSGVRIQRKKEKMKET(SEQ ID NO: 1152) |
| Construct III (hep/col IV) | mIL2-VRIQRKKEKMKET(SEQ ID NO: 1139)- |
| | MMPcs1-GGSKLWVLPKGS(SEQ ID NO: 1155)- |
| | mIL2Ra-muIgG1Fc |
| Construct JJJ (col IV/hep) | mIL2-KLWVLPKGGS(SEQ ID NO: 1146)-MMPcs1- |
| | VRIQRKKEKMKETGS(SEQ ID NO: 1151)-mIL2Ra- |
| | muIgG1Fc |
| Construct KKK (denatured | mIL2-TLTYTWSGGGS(SEQ ID NO: 1153)- |
| collagen) | MMPcs1-2x(G4S)(SEQ ID NO: 1142)-mIL2Ra(1-215)- |
| | muIgG1Fc |
| Construct LLL | mIL2-VRIQRKKEKMKET(SEQ ID NO: 1139)- |
| | MMPcs1-VRIQRKKEKMKET(SEQ ID NO: 1139)- |
| | mIL2Ra-muIgG1Fc |
| Construct MMM | mIL2-LHERHLNNNG(SEQ ID NO: 1147)-MMPcs1- |
| | VRIQRKKEKMKET(SEQ ID NO: 1139)-mIL2Ra- |
| | muIgG1Fc |
| Construct CCC (pH heparin) | hIL2(C125S)-2x(GHHPH)(SEQ ID NO: 1141)- |
| | MMPscr-2x(G4S)(SEQ ID NO: 1142)-hIL2Ra(M25I)- |
| | GSGGGG(SEQ ID NO: 1138)-huIgG1Fc(LALA) |
| Construct DDD (pH heparin) | hIL2(C125S)-2x(GHHPH)(SEQ ID NO: 1141)- |
| | MMPcs1-2x(G4S)(SEQ ID NO: 1142)-hIL2Ra(M25I)- |
| | GSGGGG(SEQ ID NO: 1138)-huIgG1Fc(LALA) |
| Construct EEE (heparin) | hIL2(C125S)-VRIQRKKEKMKET(SEQ ID NO: |
| | 1139)-MMPcs1-2x(G4S)(SEQ ID NO: 1142)- |
| | hIL2Ra(M25I)-GSGGGG(SEQ ID NO: 1138)- |
| | huIgG1Fc(LALA) |
| Construct FFF (heparin) | hIL2(C125S)-VRIQRKKEKMKET(SEQ ID NO: |
| | 1139)-MMPscr-2x(G4S)(SEQ ID NO: 1142)- |
| | hIL2Ra(M25I)-GSGGGG(SEQ ID NO: 1138)- |
| | huIgG1Fc(LALA) |
| Construct NNN col IV | huIL2(C125S)-SGGKLWVLPK(SEQ ID NO: 1154)- |
| | MMPcs1-2x(G4S)(SEQ ID NO: 1142)-huIL2Ra(1-219; |
| | M25I)-GSGGGG(SEQ ID NO: 1138)-huIgG1(LALA) |
| Construct OOO hep/colIV | huIL2(C125S)-VRIQRKKEKMKET(SEQ ID NO: |
| | 1139)-MMPcs1-GGSKLWVLPKGS(SEQ ID NO: |
| | 1155)-huIL2Ra(1-219;M25I)-GSGGGG(SEQ ID NO: |
| | 1138)-huIgG1(LALA) |
| Construct PPP | mIL2-VRIQRKKEKMKET(SEQ ID NO: 1139)- |
| | MMPcs1-LHERHLNNNG(SEQ ID NO: 1147)- |
| | mIL2Ra-muIgG1Fc |
| Construct QQQ | mIL2-LRELHLDNN(SEQ ID NO: 188)-MMPcs1- |
| | 2x(G4S)(SEQ ID NO: 1142)-mIL2Ra(1-215)- |
| | muIgG1Fc |
| Construct RRR | mIL2-2x(SG4)(SEQ ID NO: 1143)-MMPcs1- |
| | LRELHDNNG(SEQ ID NO: 1156)-mIL2Ralpha(1- |
| | 215)-muIgG1Fc |
| Construct SSS | mIL2-LRELHLDNNG(SEQ ID NO: 1157)-MMPcs1- |
| | VRIQRKKEKMKET(SEQ ID NO: 1139)-mIL2Ra- |
| | muIgG1Fc |
| Construct TTT | mIL2-VRIQRKKEKMKET(SEQ ID NO: 1139)- |
| | MMPcs1-LRELHLDNNG(SEQ ID NO: 1157)- |
| | mIL2Ra-muIgG1Fc |
| Construct VVV no TME | huIL15Ra(1-77)-linker-huIL15-(SG3)(SEQ ID |
| | NO: 1158)-GPLGVRG(SEQ ID NO: 80)-4x(G4S)(SEQ |
| | ID NO: 1142)-IL2Rb(1-214)-6xHis(SEQ ID NO: 1159) |
| Construct WWW | huIL15Ra(1-77)-linker-huIL15-(SG3)(SEQ ID |
| | NO: 1158)-GPLGVRG(SEQ ID NO: 80)- |
| | gsVRIQRKKEKMKET(SEQ ID NO: 1160)- |
| | 3x(G4S)(SEQ ID NO: 1142)-IL2Rb(1-214)-6xHis |
| | (SEQ ID NO: 1159) |
| Construct XXX | huIL15Ra(1-77)-linker-huIL15-(SG3)(SEQ ID |
| | NO: 1158)-GPLGVRG(SEQ ID NO: 80)- |
| | ggsKLWVLPK(SEQ ID NO: 1161)-2x(G4S)(SEQ ID |
| | NO: 1142)-IL2Rb(1-214)-6xHis(SEQ ID NO: 1159) |
| Construct YYY | huIL15Ra(1-77)-linker-huIL15-(SG3)(SEQ ID |
| | NO: 1158)-GPLGVRG(SEQ ID NO: 80)- |
| | VRIQRKKEKMKET(SEQ ID NO: 1139)- |
| | 2x(G4S)(SEQ ID NO: 1142)-IL2Rb(1-214)- |
| | (G4SG)(SEQ ID NO: 1162)-HuIgG1Fc |
| Construct ZZZ | huIL15Ra(1-77)-linker-huIL15-(SG3)(SEQ ID |
| | NO: 1158)-GPLGVRG(SEQ ID NO: 80)- |
| | ggsKLWVLPK(SEQ ID NO: 1161)-2x(G4S)(SEQ ID |
| | NO: 1142)-IL2Rb(1-214)-(G4SG)(SEQ ID NO: 1162)- |
| | HuIgG1Fc |
| Construct AAAA | huIL15Ra(1-77)-linker-huIL15-(SG3)(SEQ ID |
| | NO: 1158)-GPLGVRG(SEQ ID NO: 80)- |
| | gLRELHLDNN(SEQ ID NO: 1163)-2x(G4S)(SEQ ID |
| | NO: 1142)-IL2Rb(1-214)-(G4SG)(SEQ ID NO: 1162)- |
| | HuIgG1Fc |
| Construct BBBB | huIL15Ra(1-77)-linker-huIL15-(SG3)(SEQ ID |
| | NO: 1158)-GPLGVRG(SEQ ID NO: 80)- |
| | VRIQRKKEKMKET(SEQ ID NO: 1139)- |
| | ggsKLWVLPK(SEQ ID NO: 1161)-IL2Rb(1-214)- |
| | (G4SG)(SEQ ID NO: 1162)-HuIgG1Fc |
| Construct CCCC | huIL15Ra(1-77)-linker-huIL15-(SG3)(SEQ ID |
| | NO: 1158)-GPLGVRG(SEQ ID NO: 80)- |
| | ggsKLWVLPK(SEQ ID NO: 1161)-ggsKLWVLPK |
| | (SEQ ID NO: 1161)-IL2Rb(1-214)-(G4SG)(SEQ ID |
| | NO: 1162)-HuIgG1Fc |
| Construct GGGG | huIgG1Fc-VRIQRKKEKMKET(SEQ ID NO: 1139)- |
| | GPLGVRG(SEQ ID NO: 80)-hCXCL9 |
| Construct HHHH | huIgG1Fc-KLWVLPK(SEQ ID NO: 200)- |
| | GPLGVRG(SEQ ID NO: 80)-hCXCL9 |
| Construct IIII | 6xHis(SEQ ID NO: 1159)-HSA-(G4S)(SEQ ID NO: |
| | 1142)-KLWVLPK(SEQ ID NO: 200)-GPLGVRG |
| | (SEQ ID NO: 80)-hCXCL9 |
| Construct JJJJ | 6xHis(SEQ ID NO: 1159)-HSA-VRIQRKKEKMKET |
| | (SEQ ID NO: 1139)-GPLGVRG(SEQ ID NO: 80)- |
| | hCXCL9 |
| Construct KKKK | scFv Herceptin(VL-VH)-huIgG1 knob/huODC-IL2 |
| | (TME collagen IV)-huIgG1Fc hole |
| Construct LLLL | scFv Herceptin(VL-VH)-huIgG1 knob/huODC-IL2 |
| | (TME heparin)-huIgG1Fc hole |
| Construct MMMM | scFv cetuximab(VL-VH)-huIgG1 knob/huODC-IL2 |
| | (TME collagen IV)-huIgG1Fc hole |

### Example 2: Expression and Purification of Fusion Proteins

### Transient Expression of Fusion Proteins

Different mammalian cell expression systems were used to produce fusion proteins (ExpiCHO-S™, Expi293F™, Freestyle CHO-S™, and Freestyle 293™, Life Technologies). Briefly, expression constructs were transiently transfected into cells following manufacturer's protocol and using reagents provided in respective expression kits. Fusion proteins were then expressed and secreted into the cell culture supernatant. Samples were collected from the production cultures every day, and cell density and viability were assessed. Protein expression titers and product integrity in cell culture supernatants were analyzed by SDS-PAGE to determine the optimal harvesting time. Cell culture supernatants were generally harvested between 4 and 12 days at culture viabilities of typically >75%. On day of harvest, cell culture supernatants were cleared by centrifugation and vacuum filtration before further use.

### Purification of Fusion Proteins

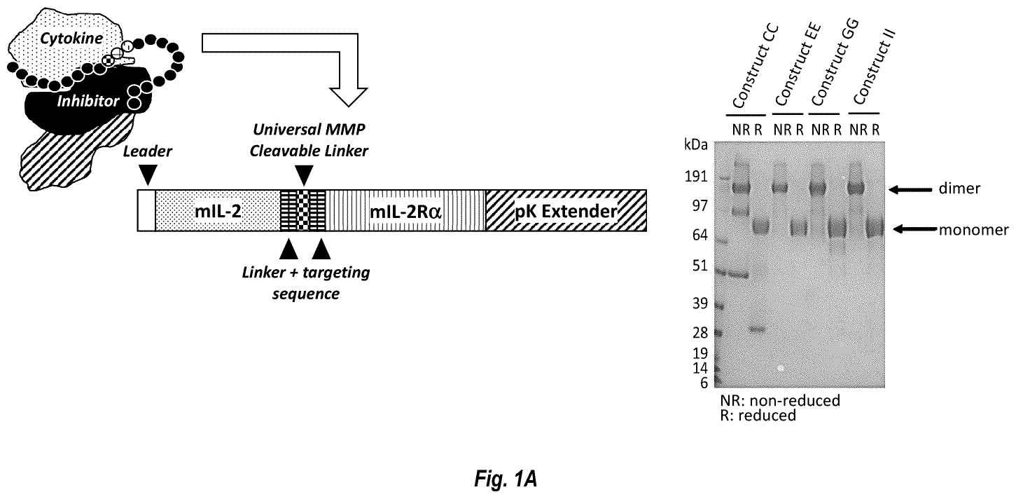

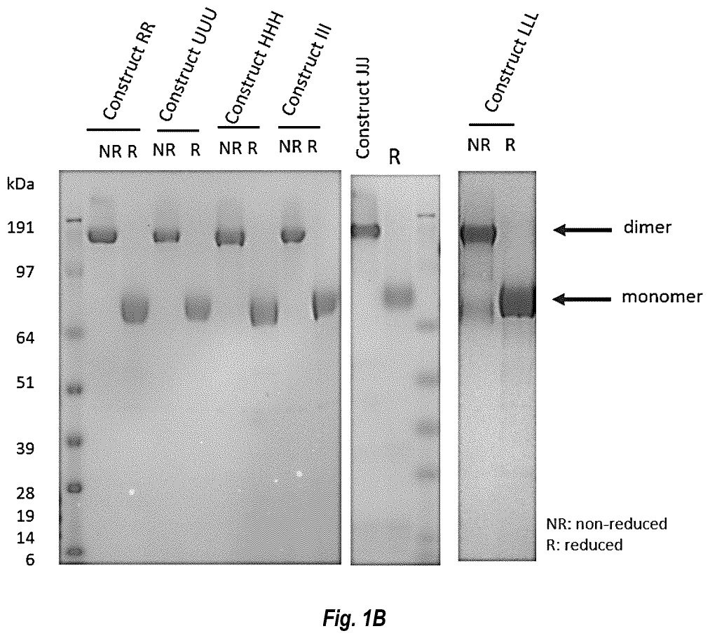

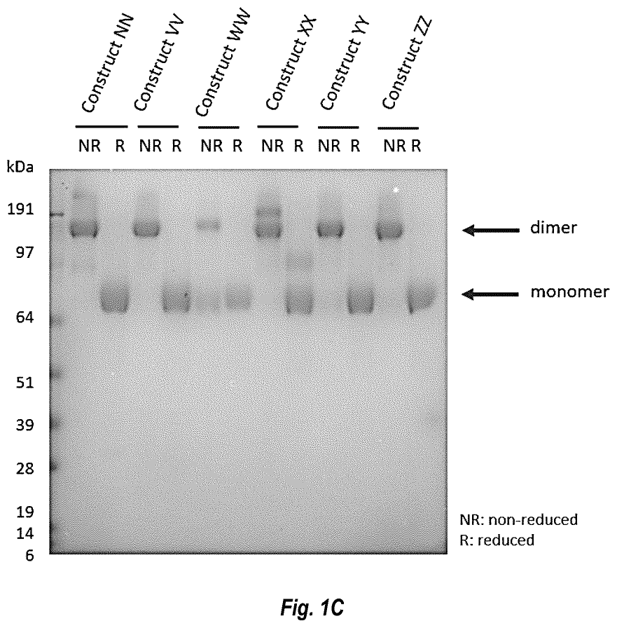

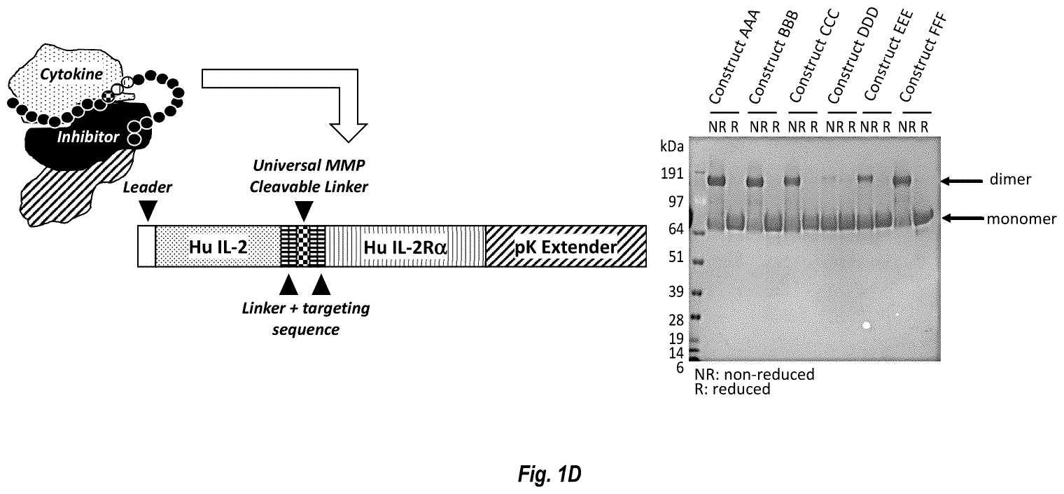

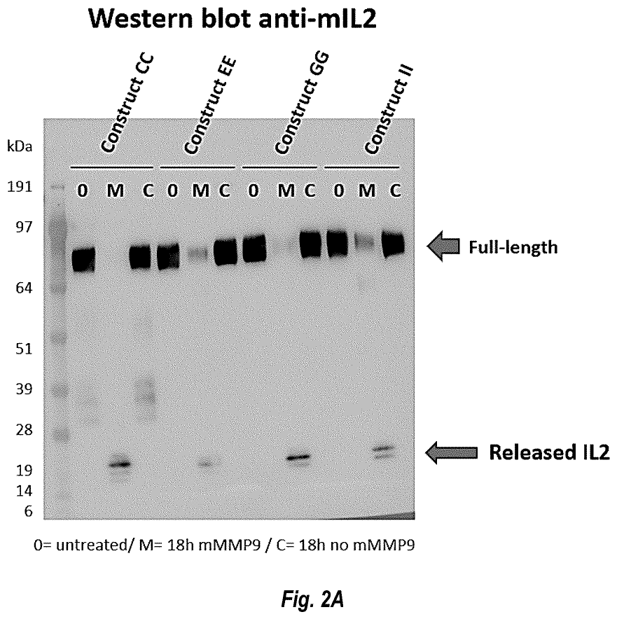

Fusion proteins were purified from cell culture supernatants in either a one-step or two-step procedure. Briefly, Fc-domain containing proteins were purified by Protein A affinity chromatography (HiTrap MabSelect SuRe, GE Healthcare). In some cases, Fc-domain containing proteins were further purified by size exclusion chromatography (HPLC SEC5 300A 7.8×300 mm, 5 m, part #5190-2526, Agilent Bio or HiLoad 26/60 Superdex 200). His-tagged proteins were first purified on a Nickel-agarose column (Ni-Penta™ Agarose 6 Fast Flow column, PROTEINDEX™), followed by size exclusion chromatography (HPLC SEC5 300A 7.8×300 mm, 5 m part #5190-2526, Agilent Bio). All purified samples were buffer-exchanged and concentrated by ultrafiltration to a typical concentration of >1 mg/mL. Purity and homogeneity (typically >90%) of final samples were assessed by SDS-PAGE under reducing and non-reducing conditions. Purified proteins were aliquoted and stored at −80° C. until further use. FIGS. 1A-1D show examples of successfully purified fusion proteins. In FIGS. 1A-1D, analysis (by Coomassie stain) of fusion proteins purified by Protein A column showed high purity of the target proteins and minimal high molecular weight entities.

### Example 3: Cleavage of Fusion Protein by MMP9 Protease

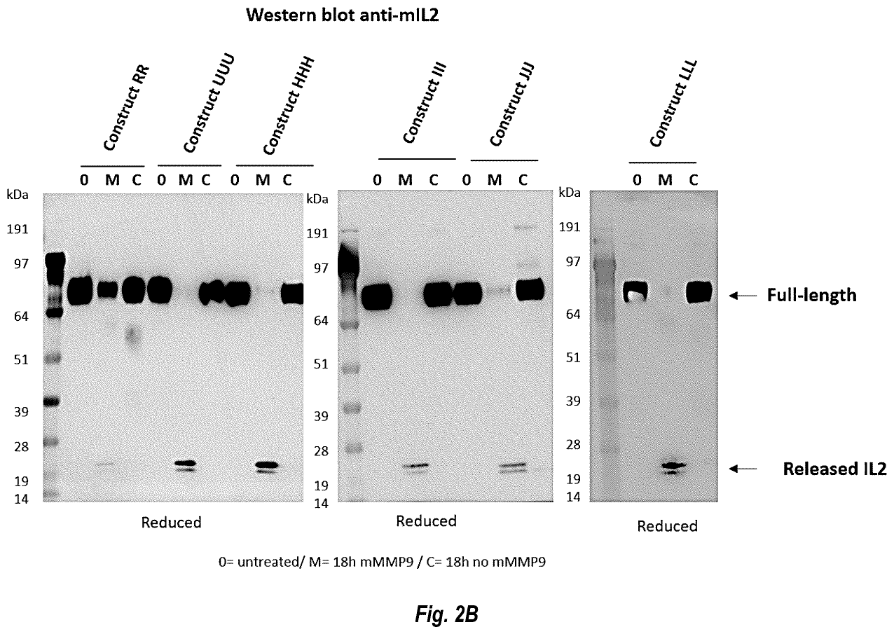

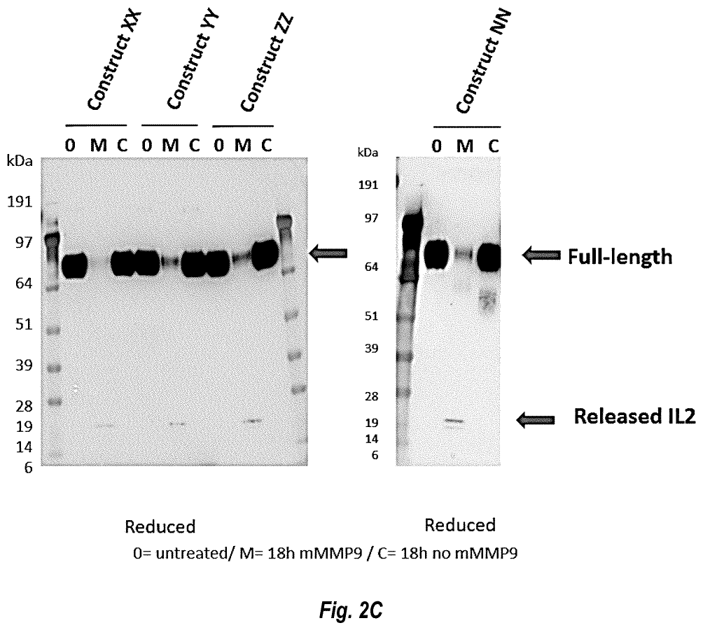

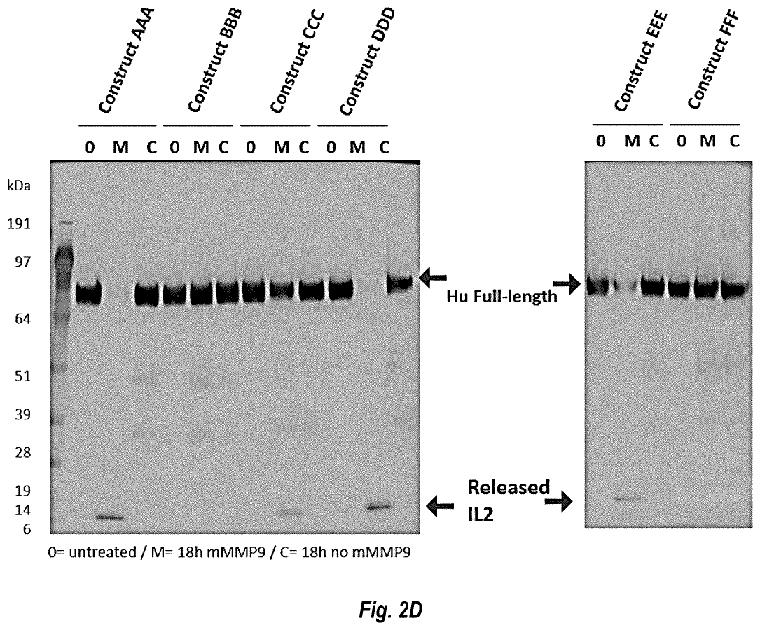

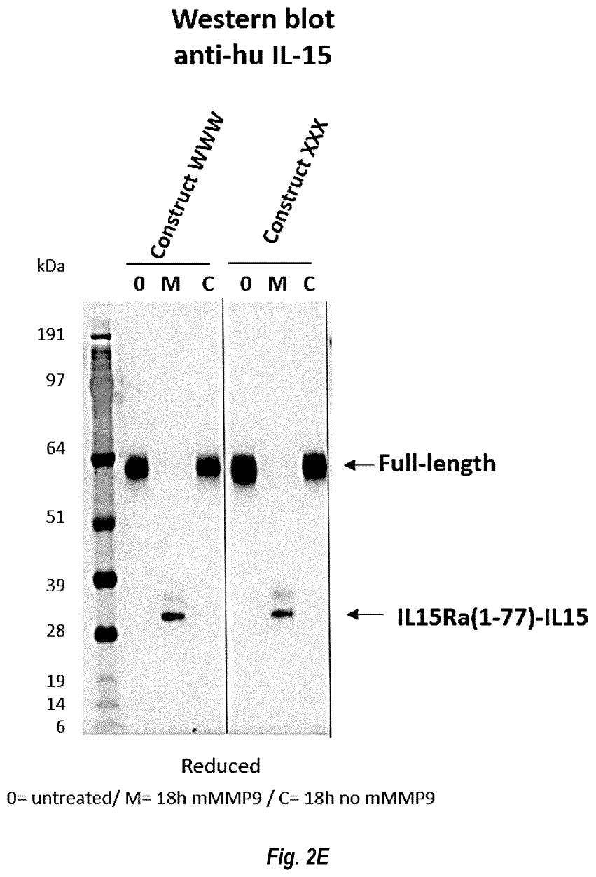

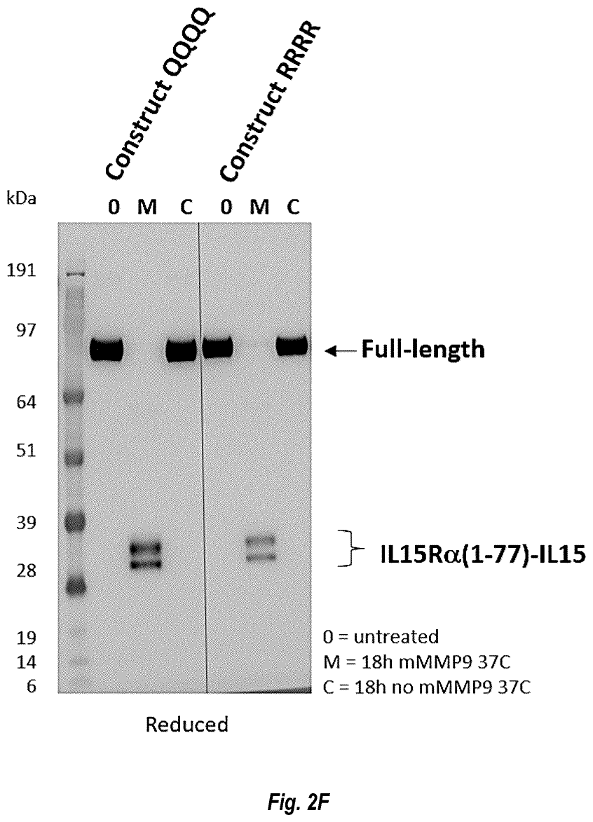

Recombinant MMP9 (R&D Systems) was first activated with p-aminophenylmercuric acetate, and this activated protease or equivalent amount of activating solution without the protease was used to digest or mock-digest the fusion protein overnight (18-22 hr) at 37° C. Cleavage assays were set up in TCNB buffer: 50 mM Tris, 10 mM CaCl₂, 150 mM NaCl, 0.05% Brij-35 (w/v), pH 7.5. Digested protein was aliquoted and stored at −80° C. prior to testing. Aliquots of digests were subsequently analyzed by SDS-PAGE followed by Western blotting to evaluate the extent of cleavage. Digests were also assessed in functional assays such as HEK-Blue Interleukin reporter assays. As shown in FIGS. 2A-2F, essentially complete cleavage by MMP9 protease of the fusion proteins with functional site was seen after overnight incubation. In contrast, proteins containing a scrambled MMP cleavage site were not cut (FIG. 2D).

### Example 4: IL-2 and IL-15 Immunoblot Analyses

Untreated and digested fusion proteins were evaluated for cleavage products by Western blot. The following antibodies were used: goat anti-mouse IL-2 polyclonal antibody (AF-402-NA; R&D systems), anti-human IL-2 antibody (Invitrogen, cat #MA5-17097, mouse IgG1), and rabbit anti-human IL-15 polyclonal antibody (ThermoFisher, cat #PA5-79466). Detection was performed using either a donkey anti-goat HRP-conjugated antibody, goat anti-rabbit HRP-conjugated antibody, or goat anti-mouse HRP-conjugated (Jackson Immuno Research, West Grove, PA), and developed using the SuperSignal West Femto Maximum sensitivity detection reagent (ThermoFisher) following the manufacturer's recommendations.

### Example 5: Detection of Mouse IL-2/IL-2Ra Fusion Proteins by ELISA

An ELISA assay was developed to detect and quantify prodrug fusion proteins comprising IL-2 and IL-2Ra moieties. Wells of a 96-well plate were coated overnight with 100 μL of a rat anti-mouse IL-2 monoclonal antibody (JES6-1A12; ThermoFisher) at 1 mg/mL in PBS. After washing, wells are blocked with TBS/0.05% Tween 20/1% BSA, then fusion proteins and/or unknown biological samples were added for 1 hour at room temperature. After washing, an anti-mouse IL-2Ra biotin-labelled detection antibody (BAF2438, R&D systems) was added and binding was detected using Ultra Strepavidin HRP (ThermoFisher). The ELISA plate was developed by adding the chromogenic tetramethylbenzidine substrate (Ultra TMB, ThermoFisher). The reaction was stopped by addition of 0.5 M H₂SO₄, and the absorbance was read at 450-650 nm.

### Example 6: IL-2 and IL-15 Functional Cell-Based Assays

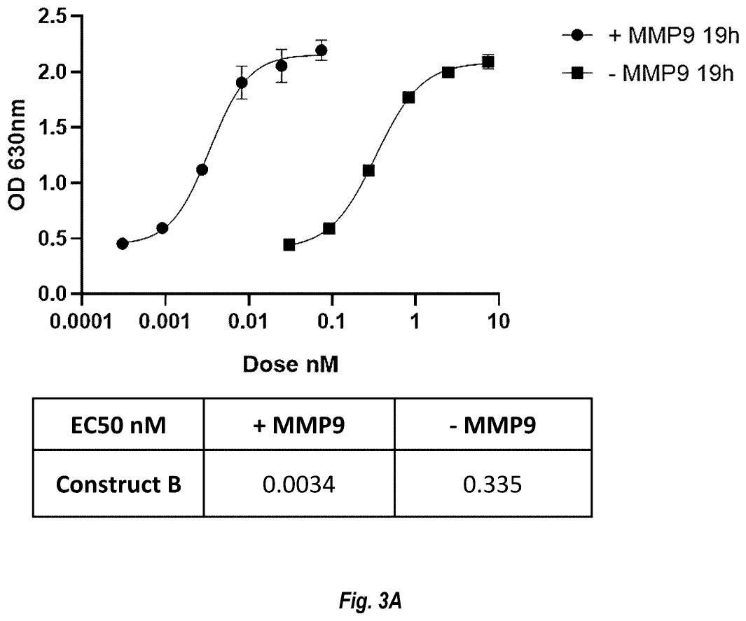

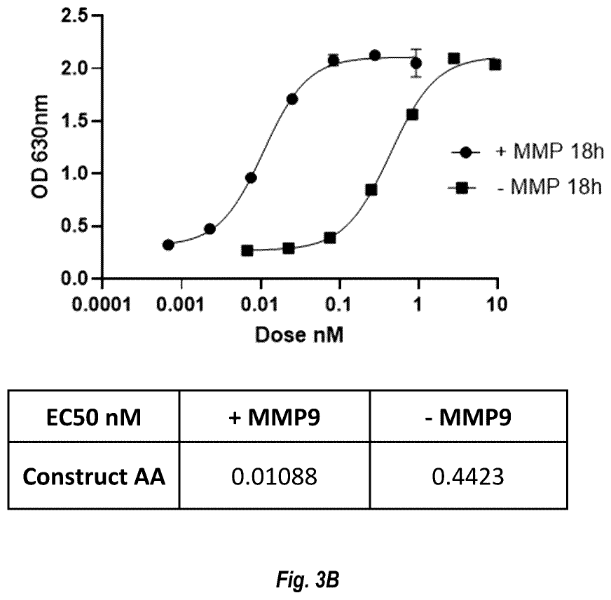

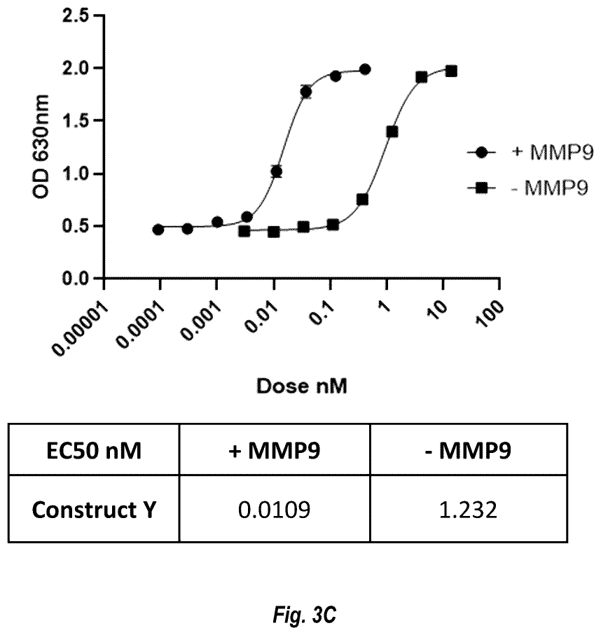

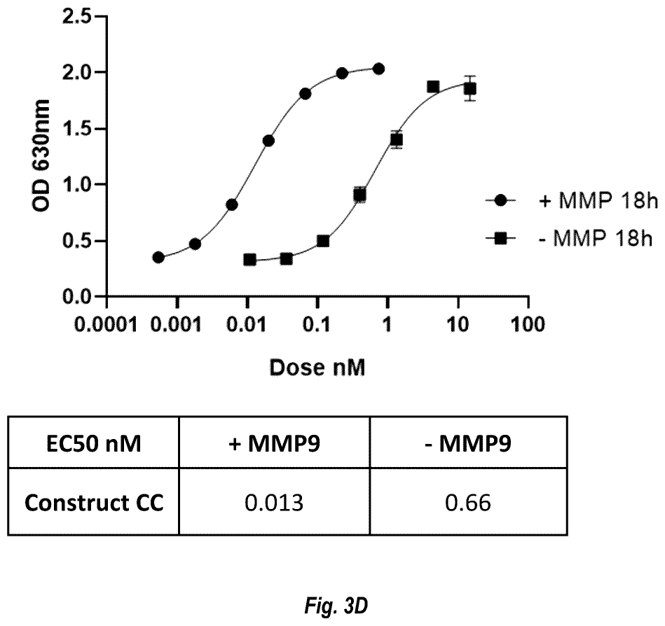

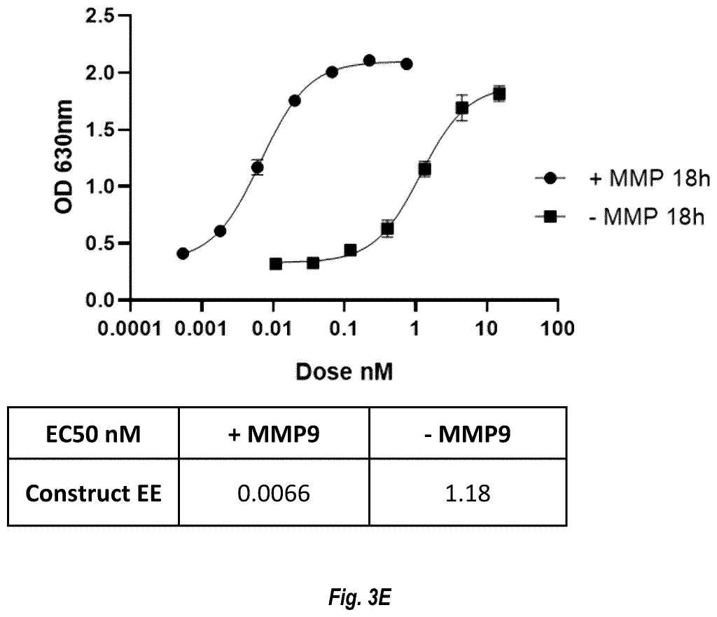

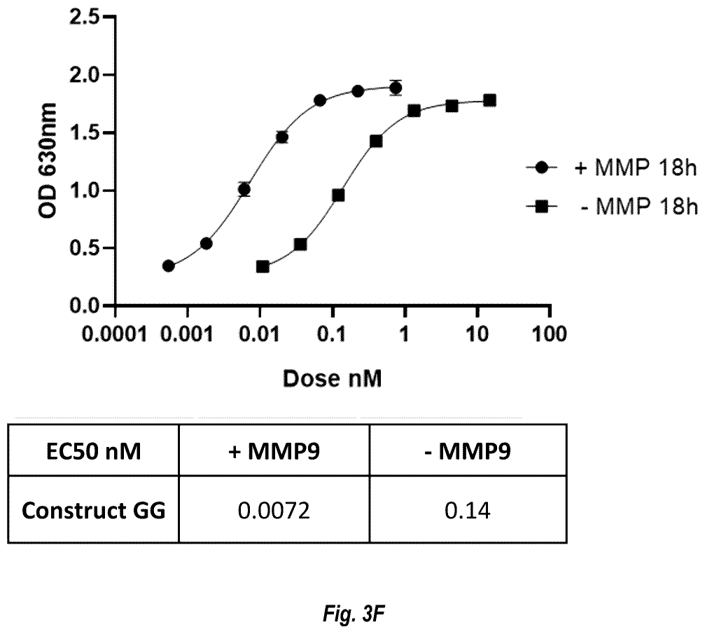

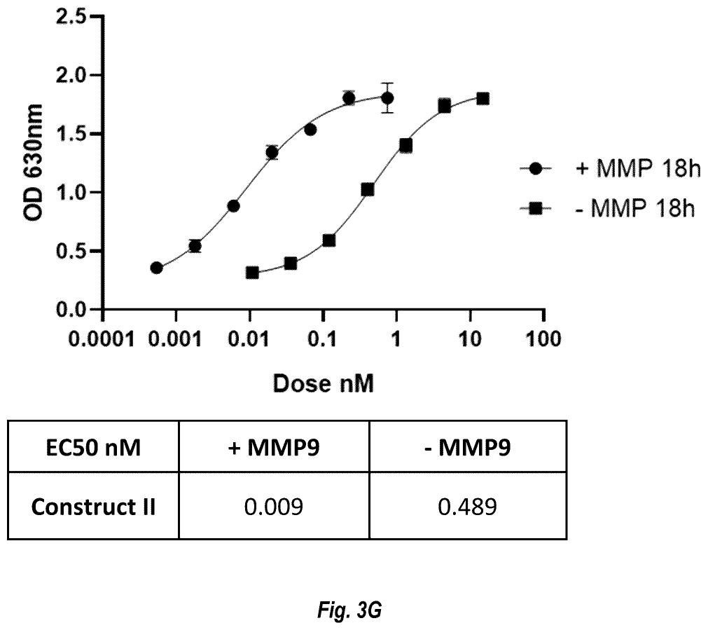

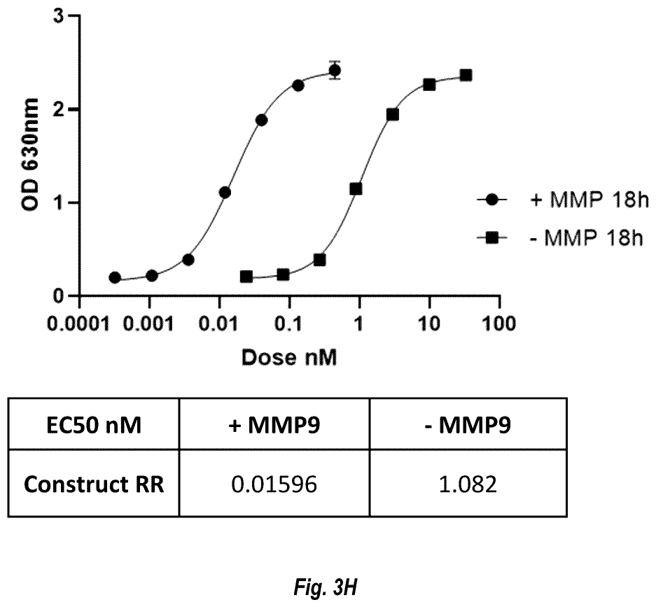

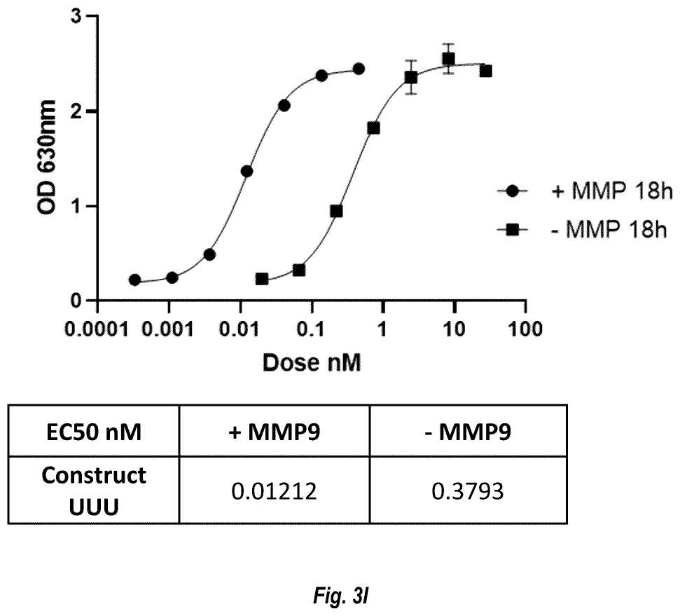

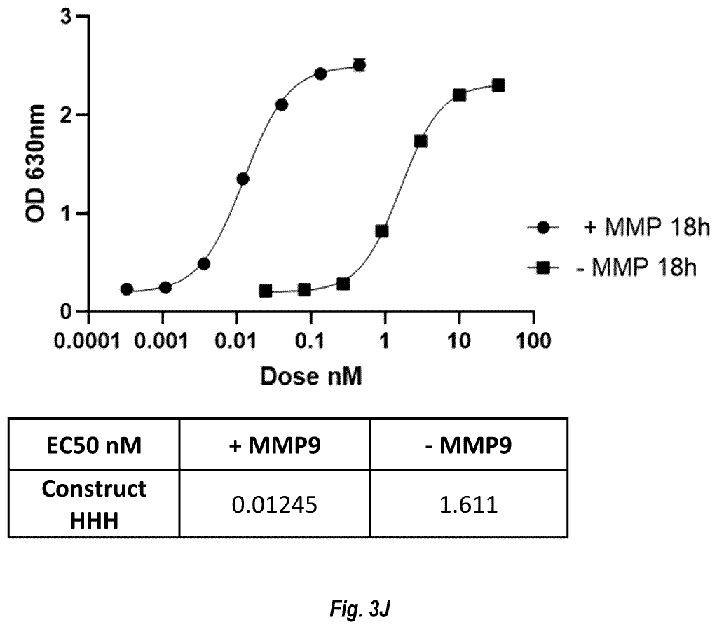

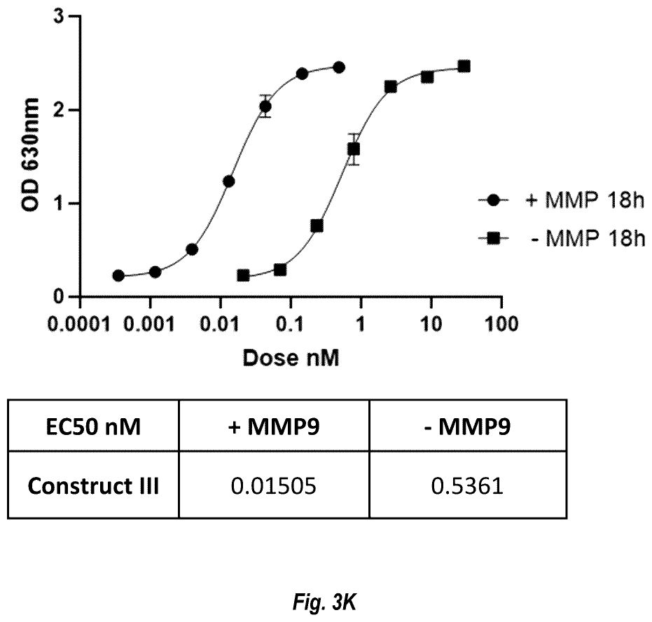

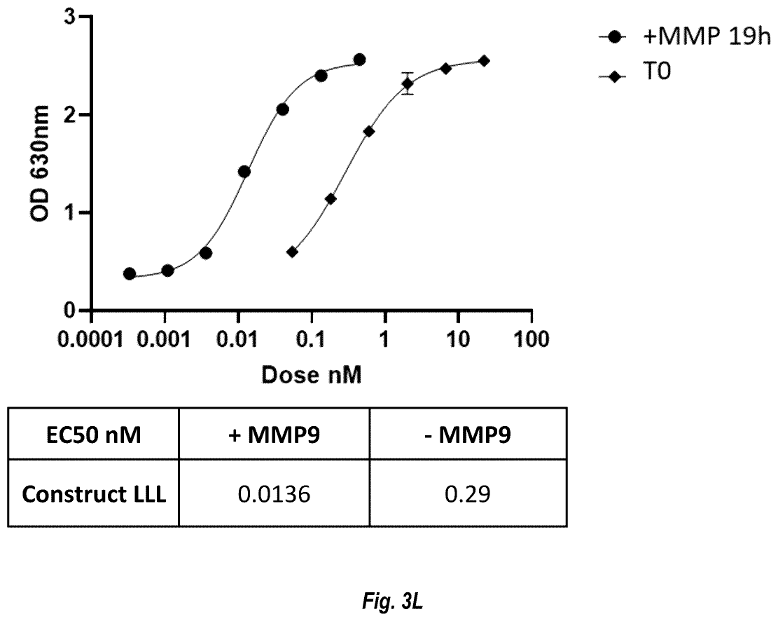

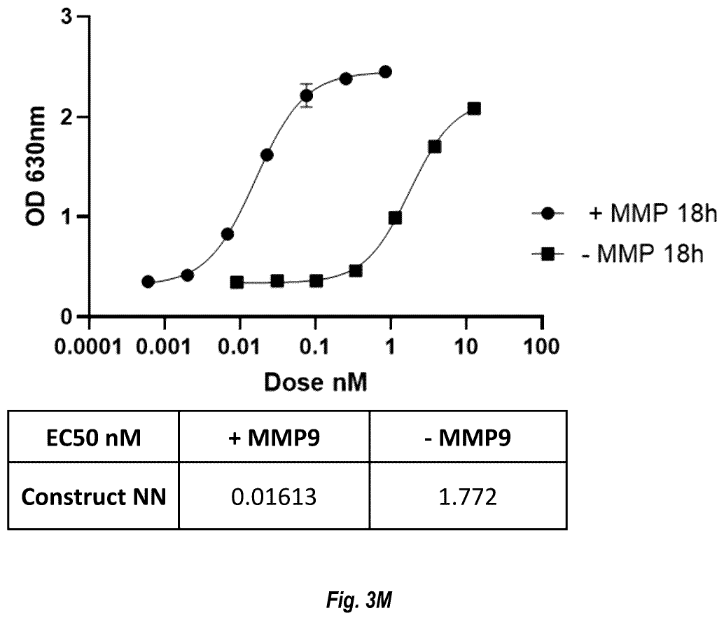

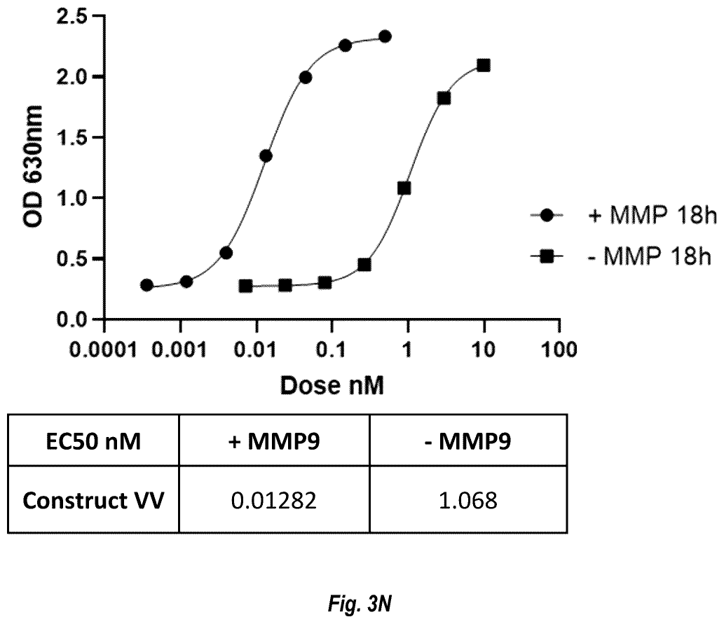

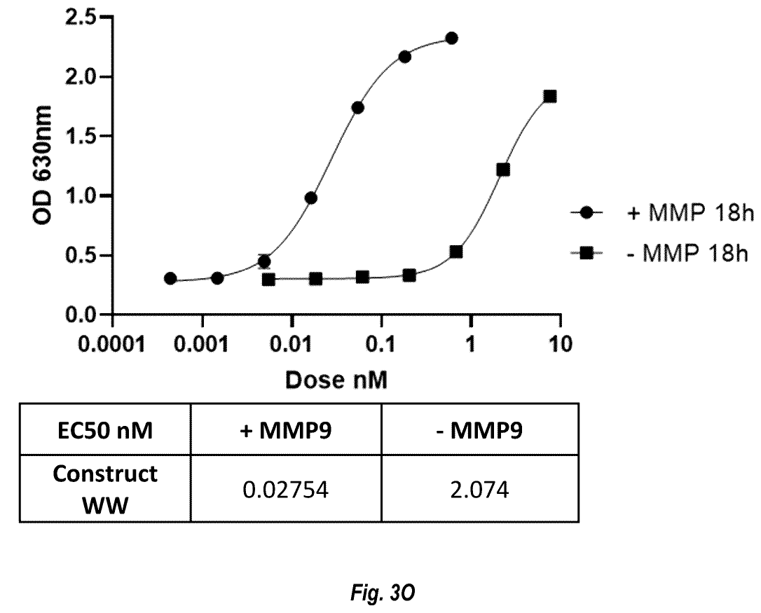

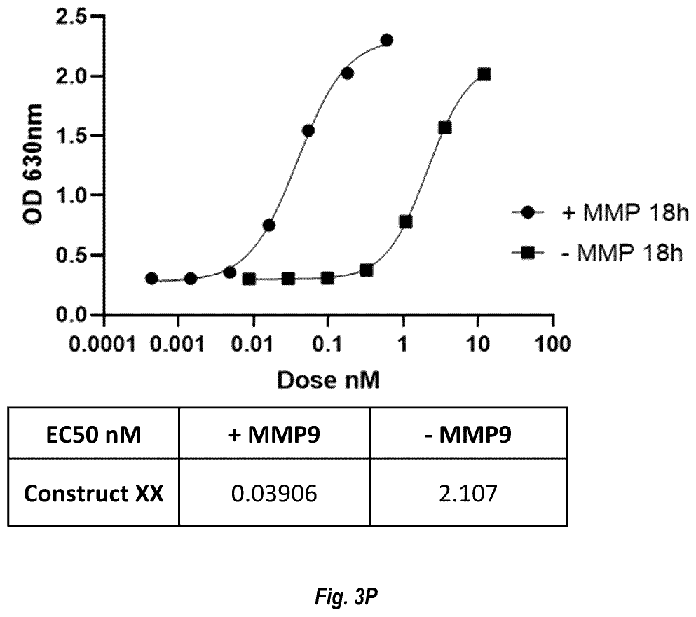

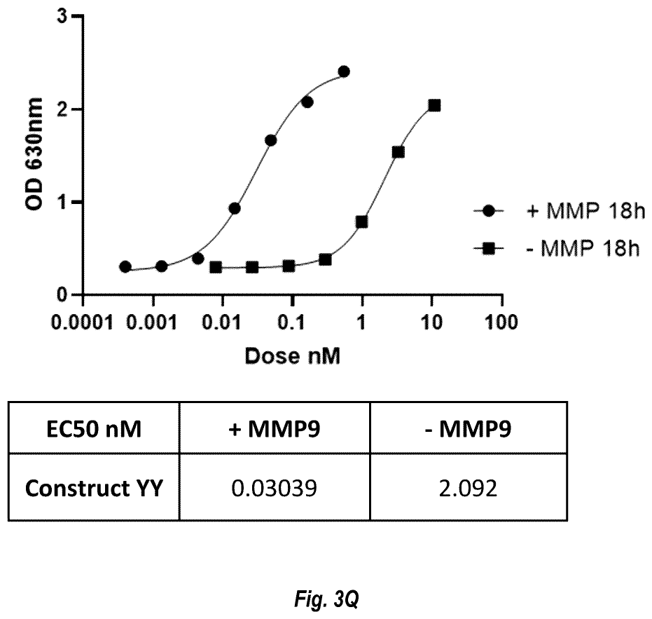

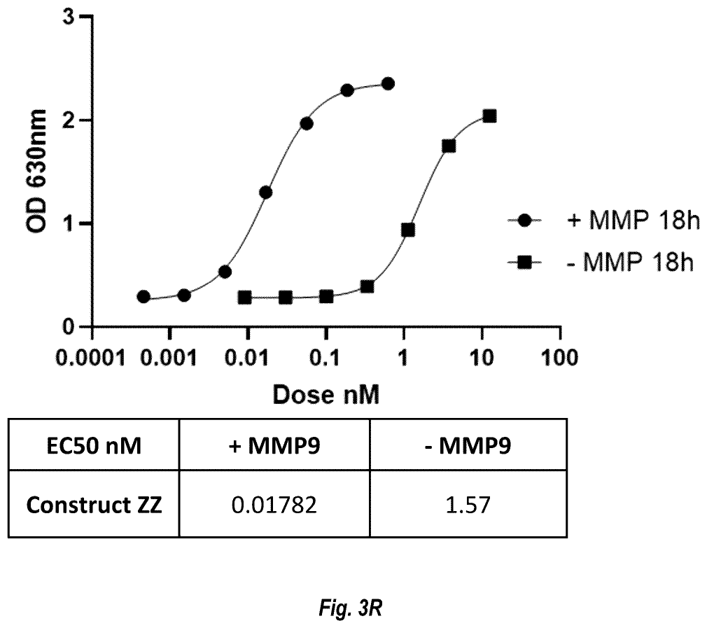

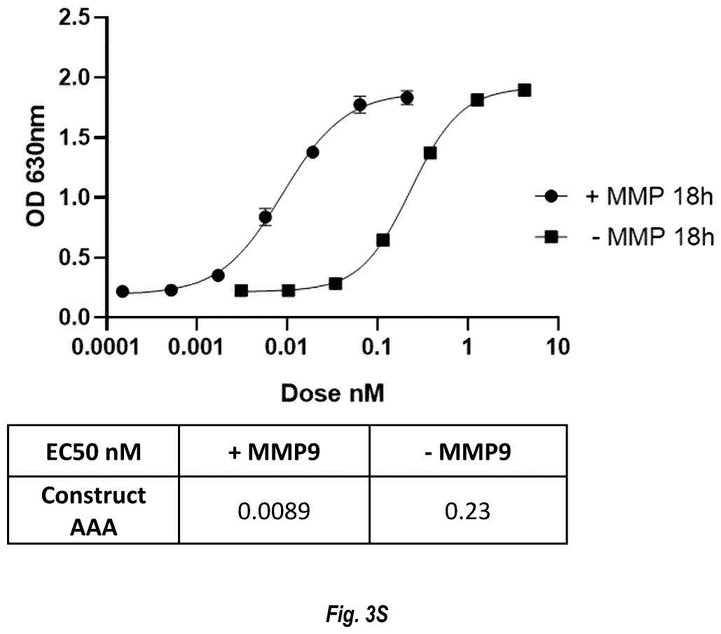

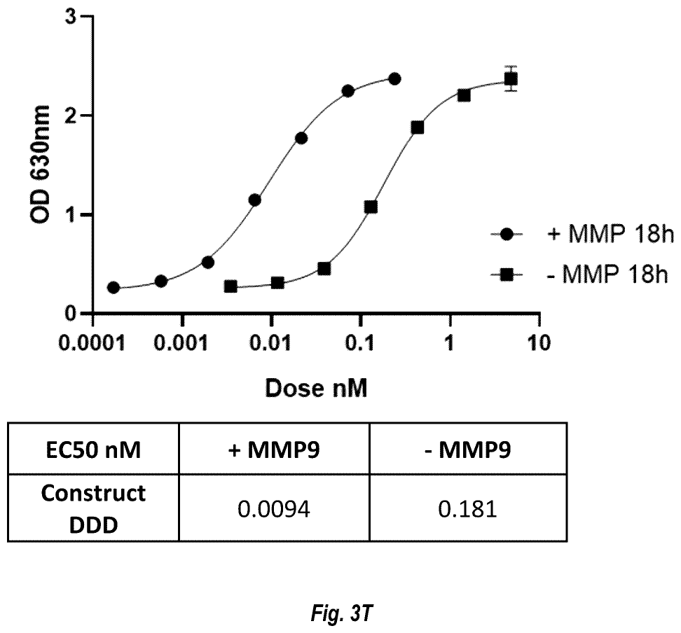

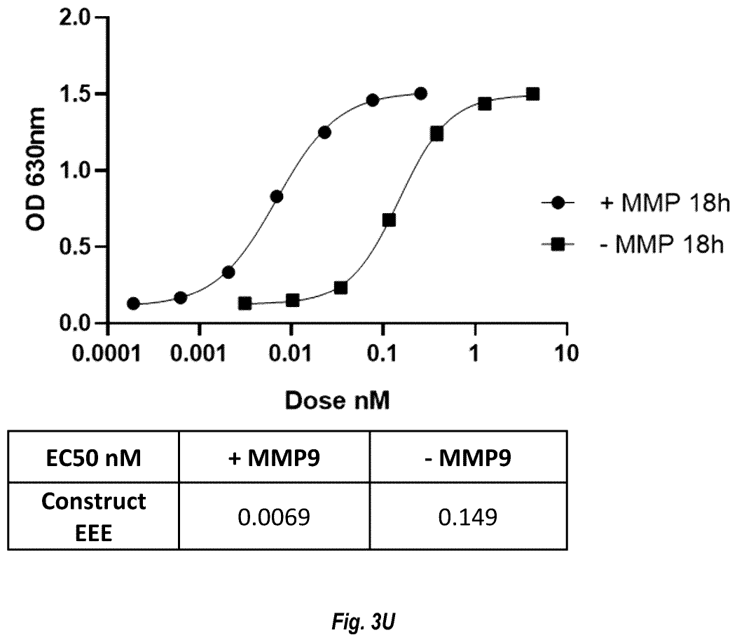

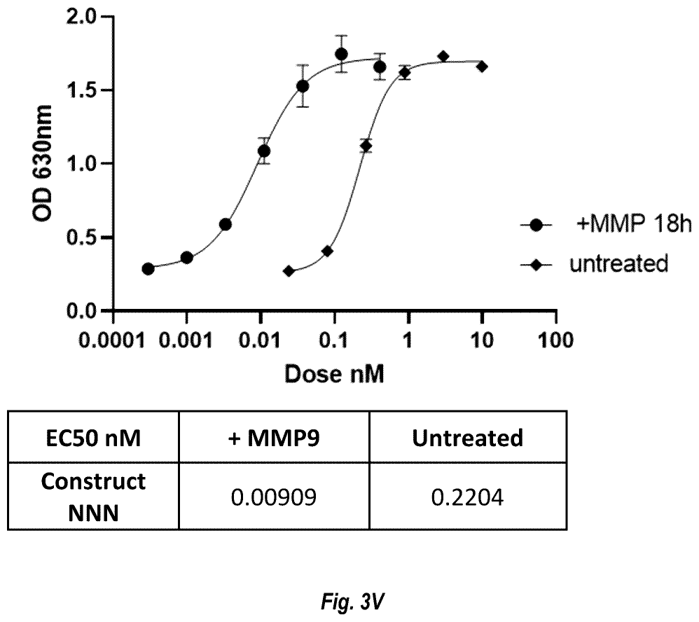

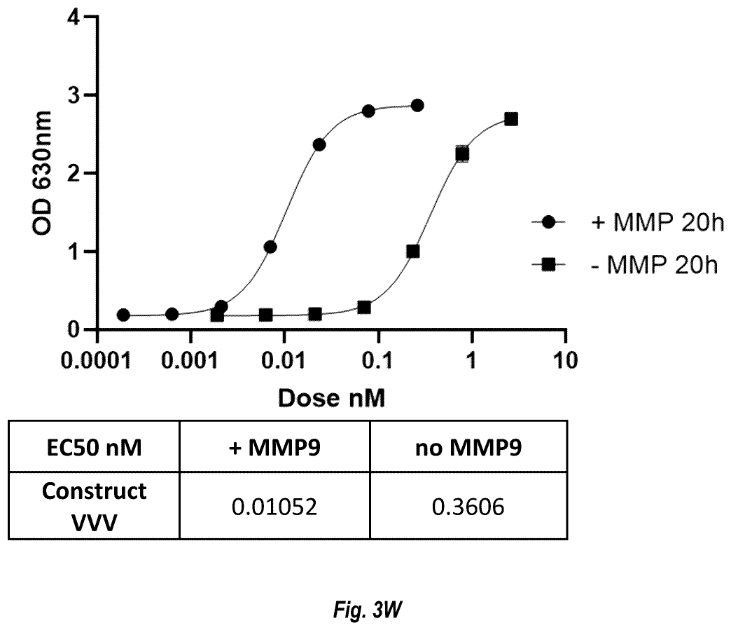

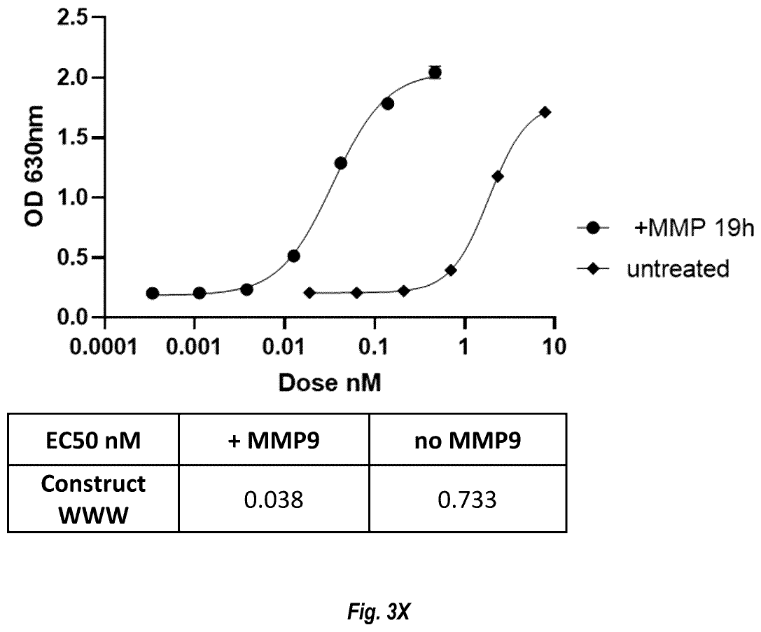

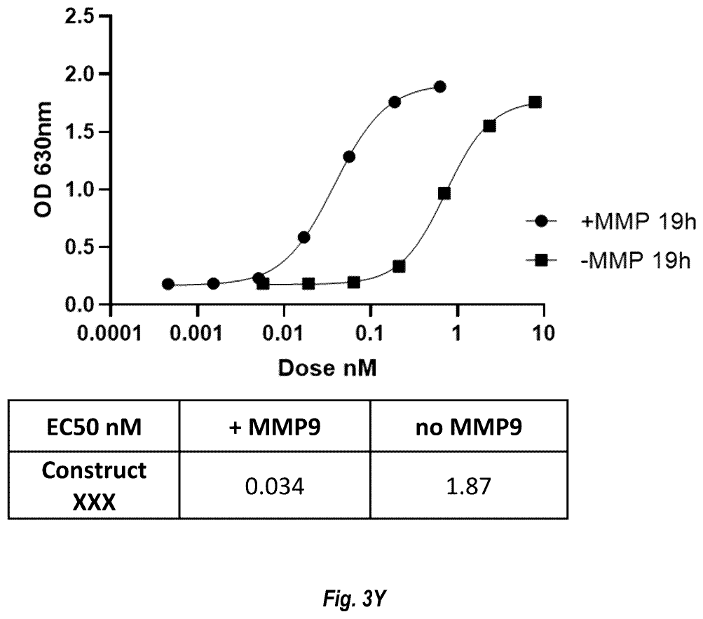

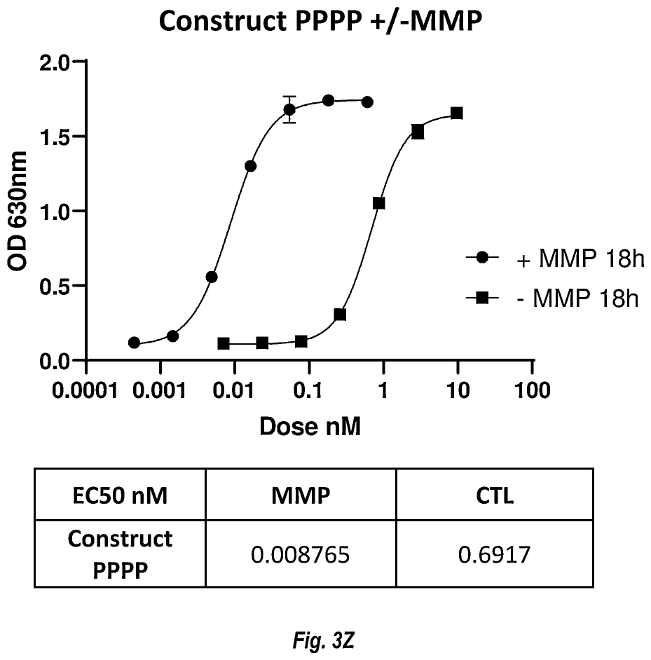

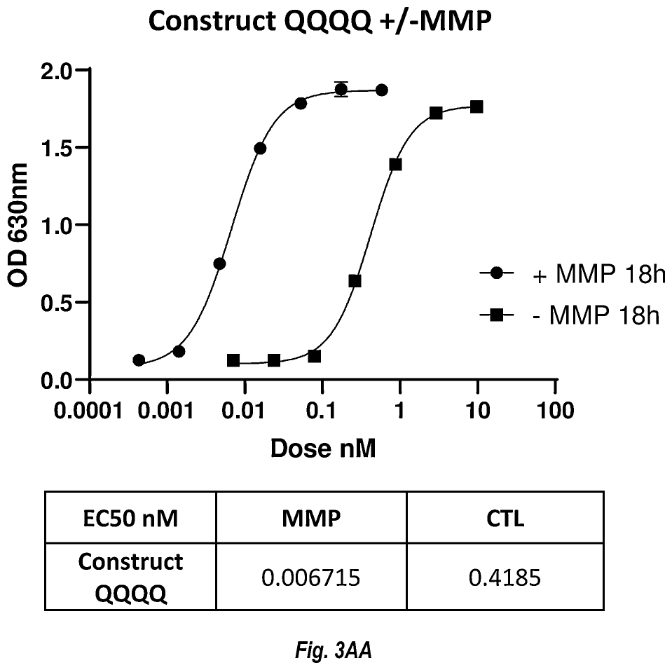

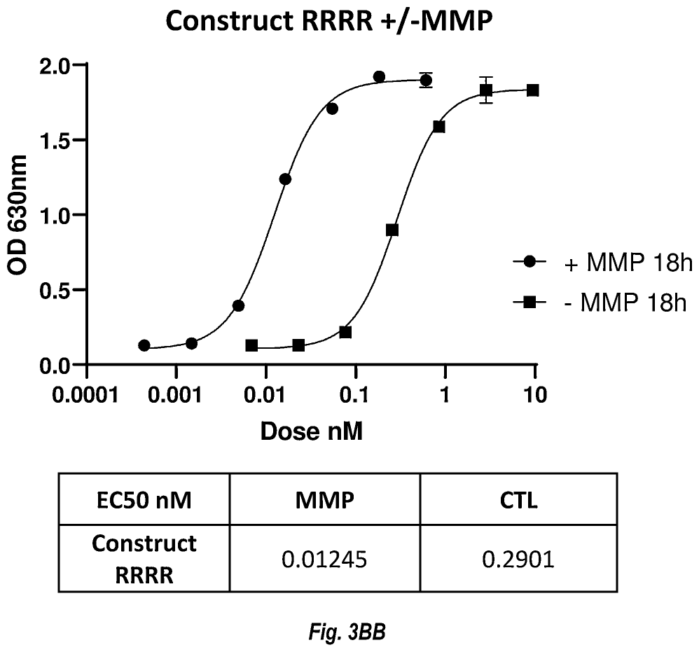

IL-2 and IL-15 are members of the four a helix bundle family of cytokines and share the same signaling receptors IL2-Rβ and common 7 chain. Hence, activity of these cytokines was measured using the same reporter cell line HEK Blue IL-2 (Invivogen, San Diego). HEK-Blue™ IL-2 cells were specifically designed to monitor the activation of the JAK-STAT pathway induced by ligand binding to the IL2-Rβ and common 7 chain receptors. Stimulation with the appropriate cytokines triggered the JAK/STAT5 pathway and induced secreted embryonic alkaline phosphatase (SEAP) production. SEAP was readily monitored using QUANTI-Blue™, a SEAP detection medium. These cells responded to human/murine IL-2 and IL-15. For the HEK Blue assay, untreated and digested samples were titrated and added to 50,000 HEK Blue cells per well in 200 μL medium in a 96-well plate and incubated at 37° C. in 5% CO₂ for 20-24 hours. The following day, levels of SEAP were measured by adding 20 μL of cell supernatant to QuantiBlue reagent, followed by 1-3 hours of incubation at 37° C. and reading absorbance at 630 nm. FIGS. 3A-3V and FIGS. 3W-3BB respectively show results obtained from IL-2 and IL-15 fusion proteins tested in HEK Blue IL-2 cell assay.

### Example 7: Next Generation Targeting Sequence Linker Peptide Binding Assay

A series of peptides comprising an MMP cleavable site with or without the addition of a targeting sequence were synthesized and conjugated to the fluorophore EDANS (5-((2-Aminoethyl)amino)naphthalene-1-sulfonic acid) (custom synthesis, ThermoFisher). Table 5 shows the list of peptides. These peptides were then tested for their ability to bind ECM proteins such as heparin, fibronectin and collagen which are found in abundance in tumor stroma. In Table 5, the bold text shows MMP cleavage site, the underlined text shows retention motif (targeting sequence) when present, and the italicized asterisk (*) shows Edans fluorophore conjugated to peptide.

TABLE 5
Next generation MMP cleavable linkers with targeting sequences

| | | SEQ ID | |
|---|---|---|---|
| Peptide | Sequence | NO: | Target of Targeting Sequence |
| 1 | GGGSGGGGPLGVRG-* | 666 | None (1^{st} gen) |
| 2 | GGGHHPHGPLGVRG-* | 667 | pH dependent heparin |
| 3 | GVRIQRKKEKMKET-* | 668 | heparin |
| 4 | FHRRIKAGPLGVRG-* | 669 | heparin |
| 7 | GGGSGGGPAALIGG-* | 670 | None (1^{st} gen) |
| 13 | GGGWSHWGPLGVRG-* | 671 | pH dependent fibronectin |
| 14 | KLWVLPKGPLGVRG-* | 672 | Collagen IV |
| 15 | GGGSGLHERHLNNN-* | 673 | Collagen I |

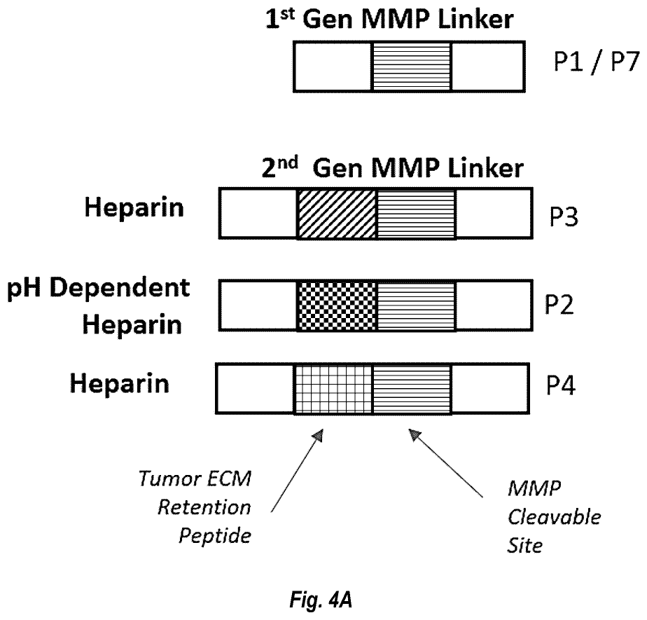

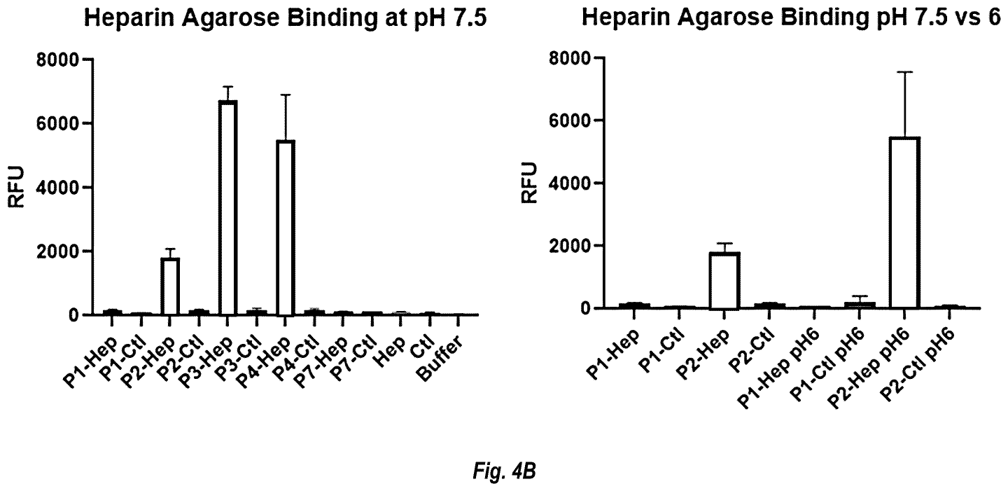

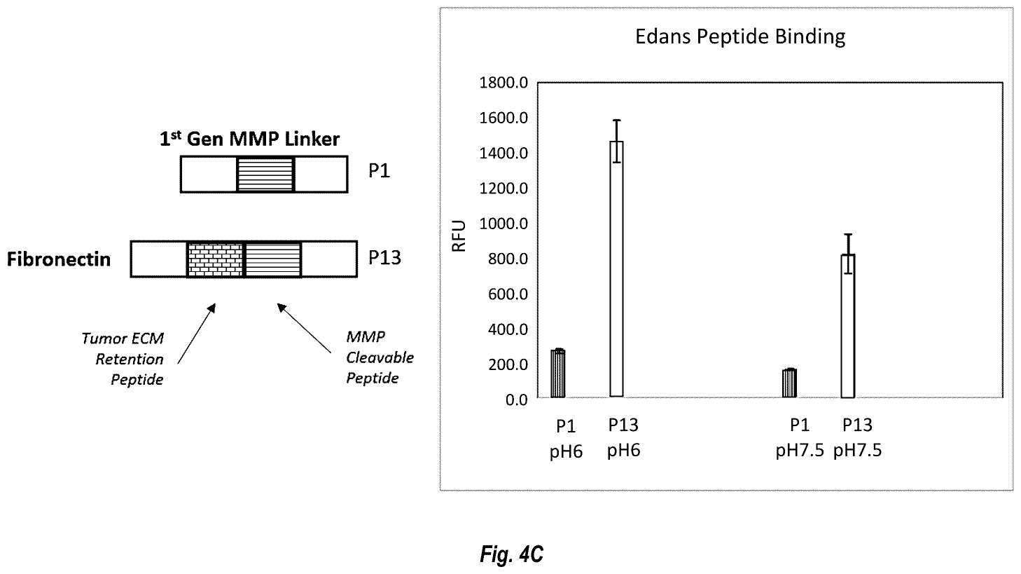

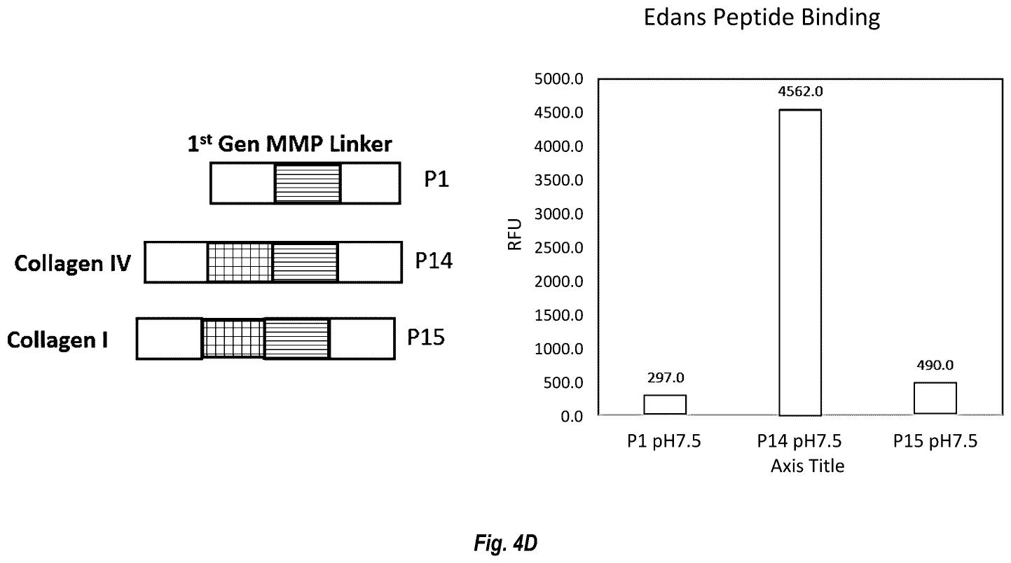

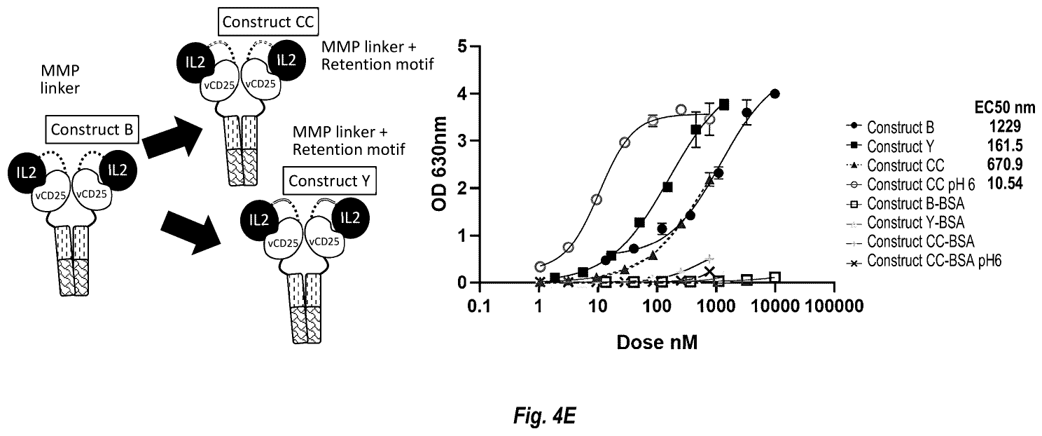

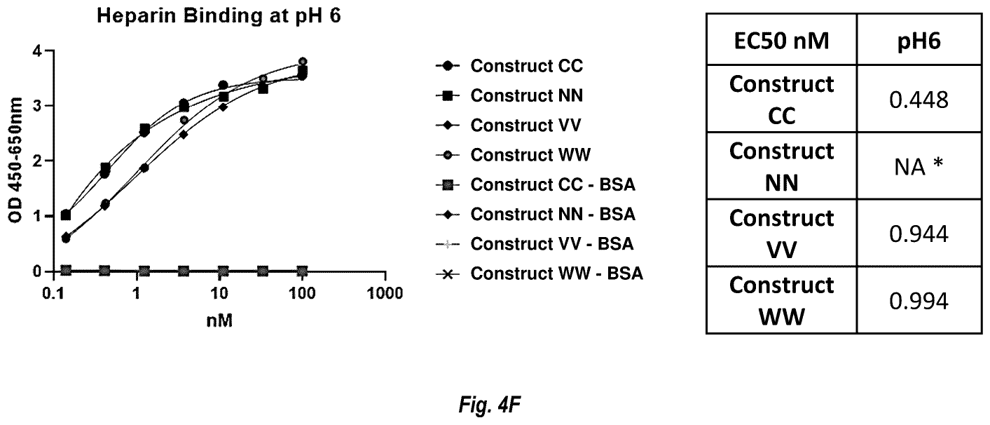

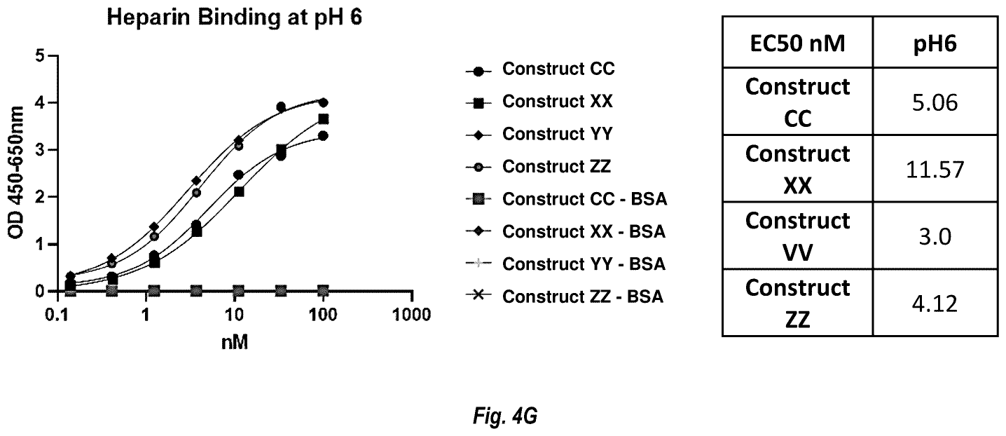

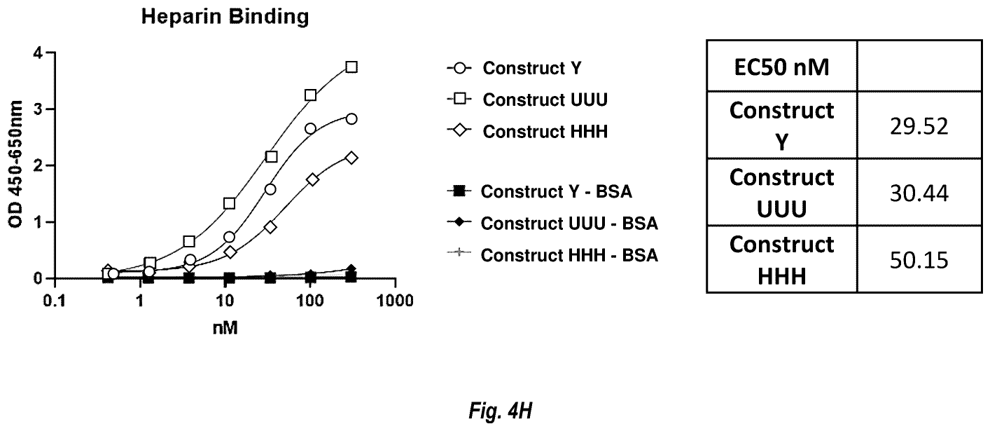

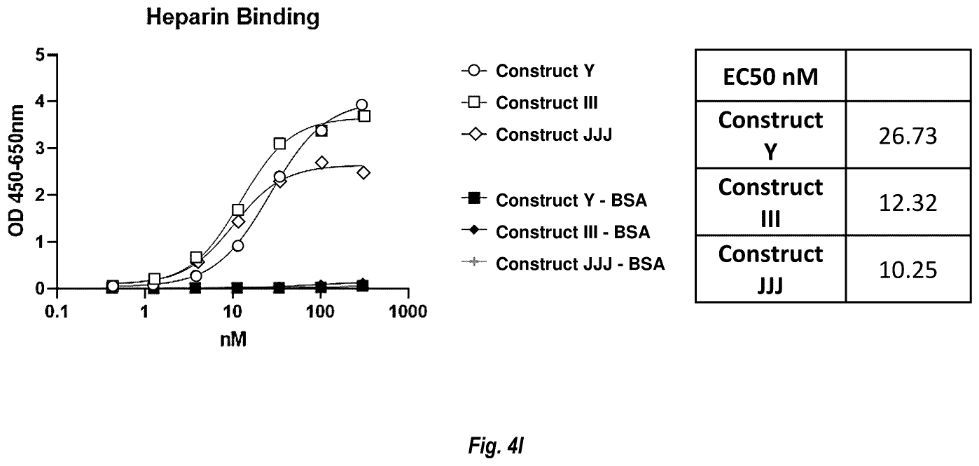

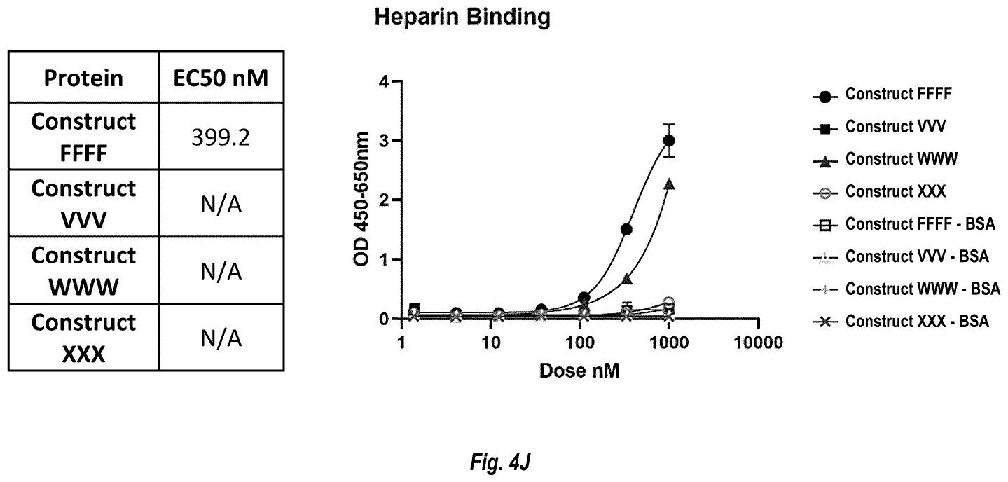

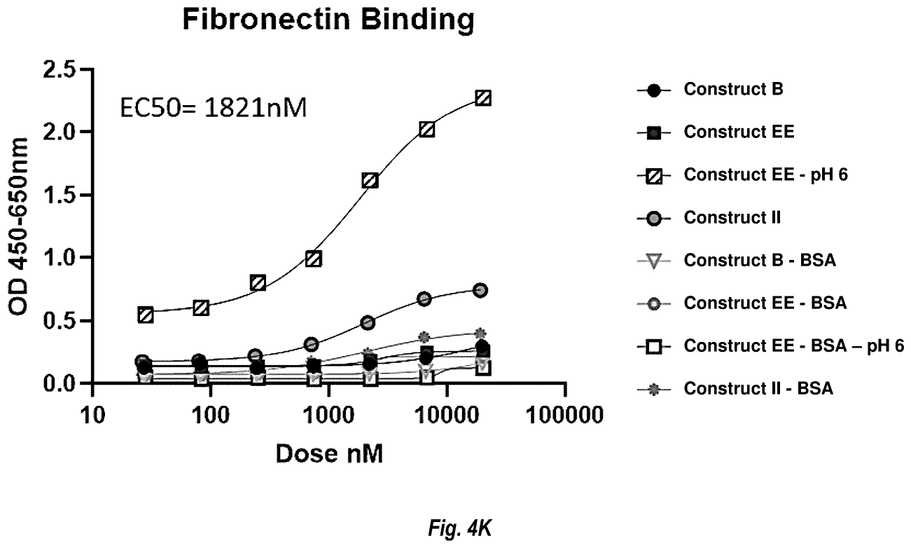

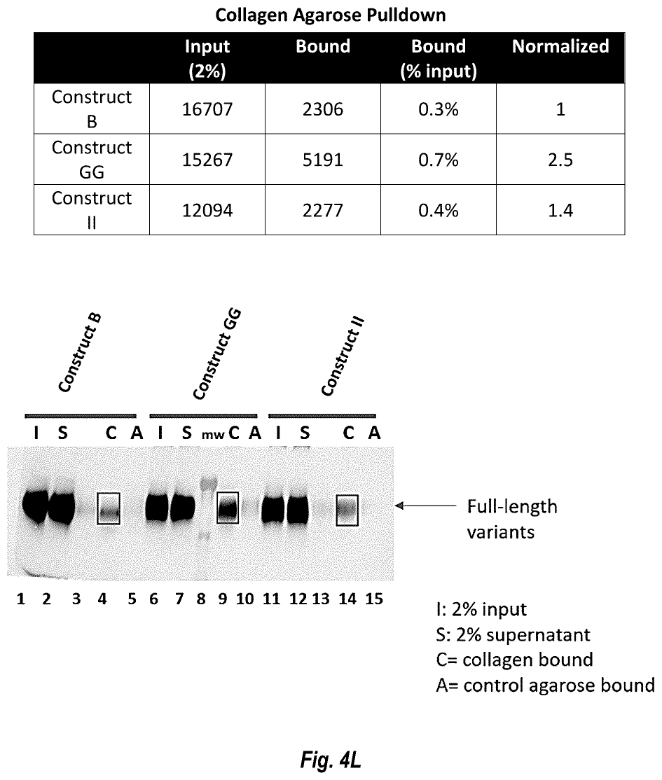

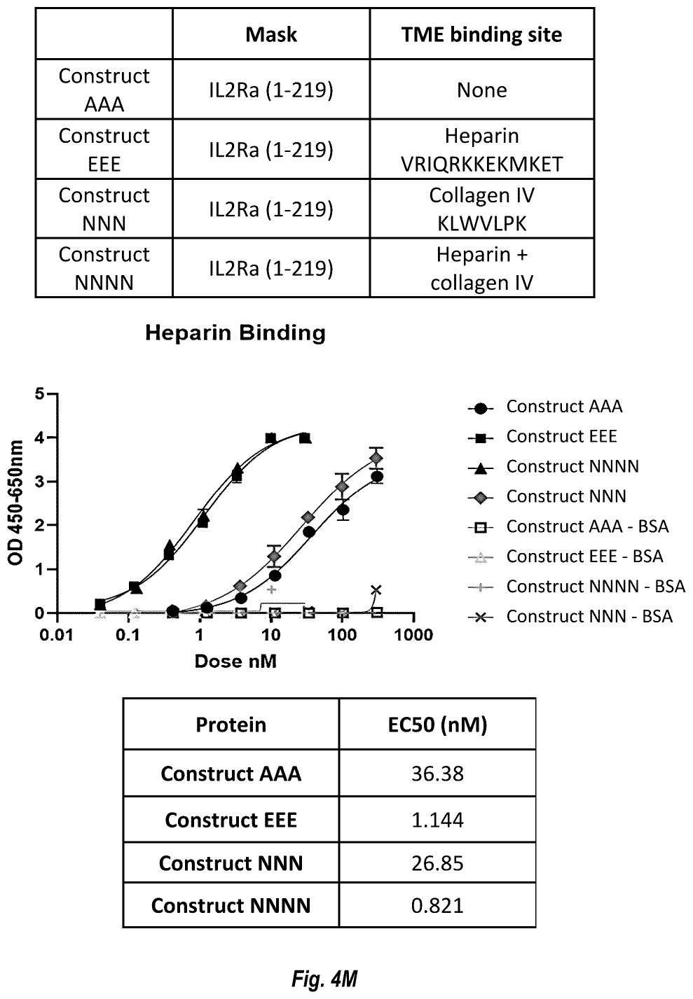

All binding assays were set up in 10 mM TrisHCl, pH 7.5 and/or 10 mM TrisHCl, pH 6. Peptides (20 μM) were incubated on a shaker for 2 hours at room temperature with agarose cross-linked to heparin or control agarose beads (Sigma and Pierce respectively). The beads were then washed 4 times and resuspended in 100 μL of binding buffer in a black 96-well plate. Peptide binding was quantified by measuring the fluorescence of samples using excitation/emission spectra of EDANS (Ex 340/Em 490). FIGS. 4A-4B show that several next generation MMP linker peptides containing heparin binding motifs bound to the heparin-agarose beads, while first generation MMP linkers lacking these targeting sequences did not. One such peptide displayed enhanced binding to heparin at pH 6 (the pH of tumors) vs. pH 7.5 (the pH of normal tissues) (FIG. 4B).

For fibronectin and collagen binding peptide assays, streptavidin coupled magnetic beads (Mag Sepharose, Cytiva and Dynabeads, ThermoFisher, respectively) were first incubated with biotin-labelled fibronectin (Cytoskeleton) or biotin-labelled collagen IV (Prospec) for 1 hour with gentle shaking. Following multiple washes, the ECM-coated beads were then incubated with Edans Peptides (20 μM) for 2 hours at room temperature with shaking in neutral or acidic binding buffer. Beads were then washed and resuspended in 100 μL of binding buffer in a black 96-well plate. Peptide binding was quantified by measuring the fluorescence of samples using excitation/emission spectra of EDANS (Ex 340/Em 490). FIG. 4C shows that peptide 13 was able to bind fibronectin and displayed enhanced binding at pH 6 (the pH of tumors) vs. pH 7.5 (the pH of normal tissues). FIG. 4D shows that peptide 14 strongly bound collagen IV, while peptide 15 bound to a lesser extent.

### Example 8: Next Generation IL-2/IL-15 Fusion Protein Binding Assays

A series of IL-2 and IL-15 fusion proteins comprising single or multiple targeting sequences in the linker regions or other locations were designed and successfully manufactured (Table 4 and FIGS. 1A-1D). These proteins were then tested for their ability to bind ECM proteins such as heparin, fibronectin, and collagen which are found in abundance in the tumor stroma.

96-well plates were coated with 10 μg/mL of Heparin-BSA conjugate (provided by Dr. Mueller, Boerhinger Ingelheim) or control BSA for 18-22 hours at room temperature on shaker (350 rpm). After washing, wells are blocked with 2% milk powder in PBS-0.05% Tween 20 or PBS-0.05% Tween 20/1% BSA for 90 minutes. The fusion proteins were then titrated in either 2% milk powder in PBS-0.05% Tween 20 or 1% BSA/PBS-0.05% Tween 20, pH 7.5 and/or pH 6, and added for 2 hours at room temperature with shaking. After washing, an anti-mouse IL-2 biotin-labelled detection antibody (JES6-5H4, ThermoFisher), anti-6×-His Tag HRP conjugate antibody (Invitrogen, 1 mg/mL, cat #MA1-21315-HRP), or anti-human IgG HRP conjugate antibody (SouthernBiotech) was added, and binding was detected using Ultra Streptavidin HRP (ThermoFisher). The plate was developed by adding the chromogenic tetramethylbenzidine substrate (Ultra TMB, ThermoFisher). The reaction was stopped by addition of 0.5 M H₂SO₄, and the absorbance was read at 450-650 nm. IL-2 fusion proteins Construct Y and Construct CC at acidic pH bound heparin in a dose-dependent manner and with higher affinity than Construct B (FIG. 4E). Strikingly, Construct CC preferentially bound heparin at acidic pH and showed the most robust binding with an EC₅₀ of about 10 nM, while Construct B's binding was much weaker, with a greater than 100-fold higher EC₅₀ value. Moreover, when the same pH-dependent heparin binding motif was inserted into different locations of IL-2 fusion proteins, all resulting proteins bound heparin at pH 6 with similar high affinities (FIGS. 4F and 4G). Likewise, similar binding affinities were observed when another heparin targeting sequence was engineered into different sites of IL-2 fusion proteins (FIGS. 4H-4I). FIG. 4J shows that IL-15Rα-IL-15 fusion protein has low intrinsic binding to heparin (EC₅₀ about 0.4 μM), an interaction which is lost when the cytokine is bound by a blocker in the context of the linker polypeptide-IL-15 fusion protein (Construct VVV). The heparin binding activity is recovered when a heparin binding motif is engineered into the linker polypeptide-IL-15 fusion protein (Construct WWW). Finally, linker polypeptide-IL-2 fusion proteins engineered with a heparin binding site show about 30-fold enhanced binding to heparin in vitro compared to constructs lacking a heparin binding site (Construct EEE and Construct NNNN vs. Construct AAA and Construct NNN, respectively) as shown in FIG. 4M.

A similar plate-based assay was developed to interrogate binding of IL-2 fusion variants to fibronectin. 96-well plates were coated with fibronectin (4-10 μg/mL, Sigma) or control BSA for 18-22 hours at room temperature on shaker (350 rpm). After washing, wells were blocked with 2% milk powder in PBS-0.05% Tween 20 or protein-free blocking buffer (Pierce) for 90 min, then fusion proteins were titrated in blocking buffer-0.1% Tween 20, pH 7.5 and/or pH 6, and added for 1 hour at room temperature with shaking. After washing, an anti-mouse IL-2 biotin-labelled detection antibody (JES6-5H4, ThermoFisher) or anti-human IgG HRP conjugate antibody (SouthernBiotech) was added, and binding was detected using Ultra Streptavidin HRP (ThermoFisher). The plate was developed by adding the chromogenic tetramethylbenzidine substrate (Ultra TMB, ThermoFisher). The reaction was stopped by addition of 0.5 M H₂SO₄, and the absorbance was read at 450-650 nm. Construct EE preferentially bound fibronectin at acidic pH and showed dose-dependent binding, while no binding was observed at pH 7.5 (FIG. 4K). No significant binding of Construct B was seen in either neutral or acidic conditions.

To test binding to collagen, a pulldown assay using agarose cross-linked to collagen (Sigma) was performed. IL-2 fusion proteins were incubated with collagen-agarose or control agarose beads for 18-22 hours at 4° C. with gentle rotation in 1% BSA/PBS-0.05% Tween 20. After washing, proteins bound to the beads were eluted by resuspending beads in SDS sample buffer (Life Technologies). Bound proteins were then separated by SDS-PAGE on 4-12% BisTris gradient gel, followed by immunoblotting with goat anti-mouse IL-2 polyclonal antibody (AF-402-NA; R&D systems). Donkey anti-goat HRP-conjugated antibody was used for detection (Jackson Immuno Research, West Grove, PA), and the blot was developed using the SuperSignal West Femto Maximum sensitivity detection reagent (ThermoFisher) following the manufacturer's recommendations. The blot image is shown in FIG. 4L. Construct GG and Construct II were specifically bound by collagen-agarose beads, while no IL-2 fusion protein bound the control agarose beads. Quantitation of the blot using iBright imaging system (Invitrogen), showed that although the fraction of bound Construct GG and Construct II was low (<1% of input), it was 2.5 and 1.4-fold higher than the fraction of bound Construct B.

### Example 9: Next Generation Retention Linker IL-2 Fusion Proteins Showed Greater Retention in Tumor In Vivo

The levels of IL-2 fusion proteins present in tumors in vivo were assessed by utilizing fluorescently labelled proteins and real-time whole-body imaging. Non-cleavable Construct GGG and Construct DD were conjugated to Dylight 650 probe according to the manufacturer's protocol (Dylight 650 Antibody labeling kit, ThermoFisher). The conjugation did not significantly alter the proteins' binding to heparin. BALB/c mice were subcutaneously inoculated with EMT6 breast cancer syngeneic model, and when the average tumor volume reached 240 mm³, animals were randomized into 3 groups based on tumor volumes (n=2 mice per treatment group). Table 6 below shows the study design.

TABLE 6
Study design for assessing IL-2 fusion proteins

| | | | | Dosing | Dose | Dose |
|---|---|---|---|---|---|---|
| | | | Dose | Frequency & | Level | Volume |
| Group | Treatment | N | Route | Duration | (mg/kg) | (mL/kg) |
| 1 | Control -PBS | 2 | IV | Once | NA | 4 |
| 2 | Construct | 2 | IV | Once | 8 | 4 |
| | GGG-DY650 | | | | | |
| 3 | Construct | 2 | IV | Once | 8 | 4 |
| | DD-DY650 | | | | | |

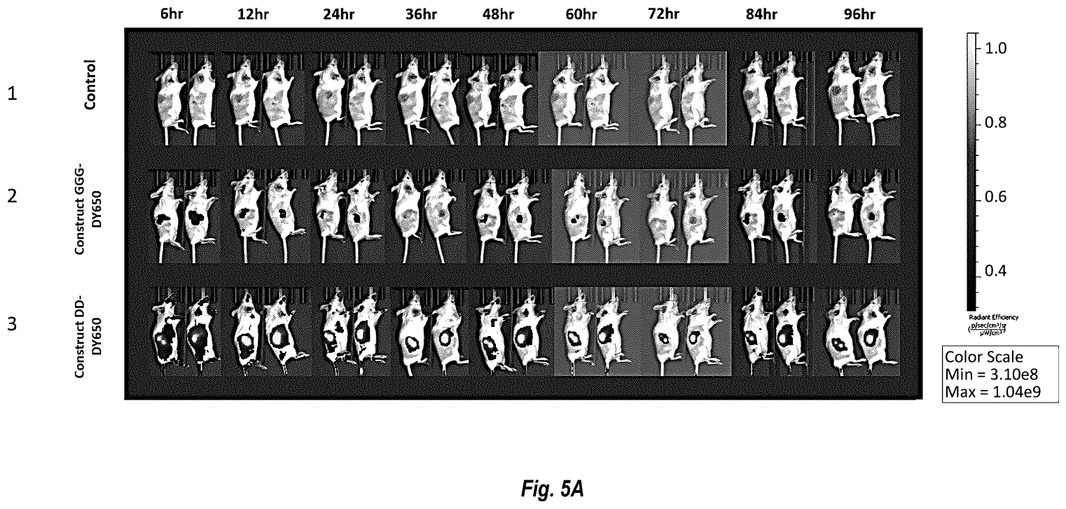

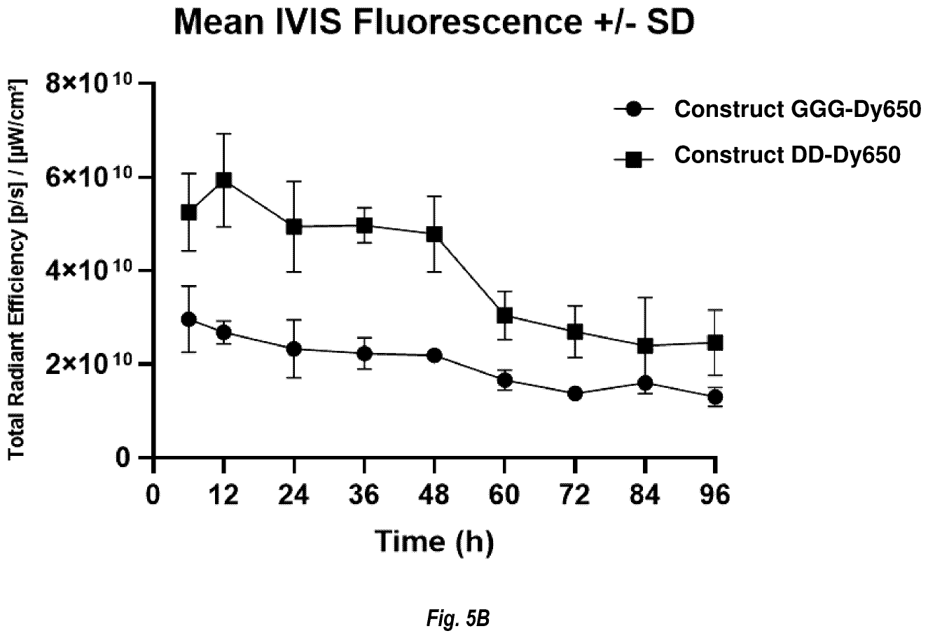

Following administration of a single dose of the labeled IL-2 fusion proteins to tumor-bearing mice, fluorescent images (excitation 640/emission 680 consistent with Dylight 650 probe ex/em spectra) were captured over 96 hours on an IVIS system (PerkinElmer, IVIS Lumina Series III) and are shown in FIG. 5A. The fluorescence intensity in tumor areas was quantified across the groups, average background tumor fluorescence (group 1) was subtracted from group 2 and 3 values at each time-point, and data were normalized to the initial fluorescence intensity of same amount of each labeled protein. FIG. 5B shows that the tumor-associated fluorescence with group 3 was roughly 2-fold higher than that of group 2 at each of the time-points tested. This signifies next generation retention linker Construct DD accumulated and was retained in tumors at 2-fold higher levels compared to IL-2 fusion protein Construct GGG, lacking any targeting sequence.

### Example 10: Multiple Targeting Sequences in Linker of IL-2 Fusion Protein Yielded Greatest Anti-Tumor Efficacy In Vivo

C57BL/6 mice were subcutaneously inoculated with B16F10 melanoma cells and when the average tumor volume reached on average 70-90 mm³, animals were randomized into 6 groups based on tumor volumes (n=8 mice per treatment group). Mice were dosed intravenously every 3 days (Q3D) for a total of 5 doses according to Table 7.

TABLE 7
Study design for assessing IL-2 fusion proteins
with multiple targeting sequences

| | | | | Dosing | Dose | Dose |
|---|---|---|---|---|---|---|
| | | | Dose | Frequency & | Level | Volume |
| Group | Treatment | N | Route | Duration | (mg/kg) | (mL/kg) |
| 1 | PBS-Vehicle | 8 | IV | Q3D for 14 | NA | 5 |
| | | | | days | | |
| 3 | Construct Y | 8 | IV | Q3D for 14 | 20 | 5 |
| | | | | days | | |
| 4 | Construct GG | 8 | IV | Q3D for 14 | 20 | 5 |
| | | | | days | | |
| 5 | Construct RR | 8 | IV | Q3D for 14 | 20 | 5 |
| | | | | days | | |
| 6 | Construct | 8 | IV | Q3D for 14 | 20 | 5 |
| | UUU | | | days | | |
| 7 | Construct III | 8 | IV | Q3D for 14 | 20 | 5 |
| | | | | days | | |

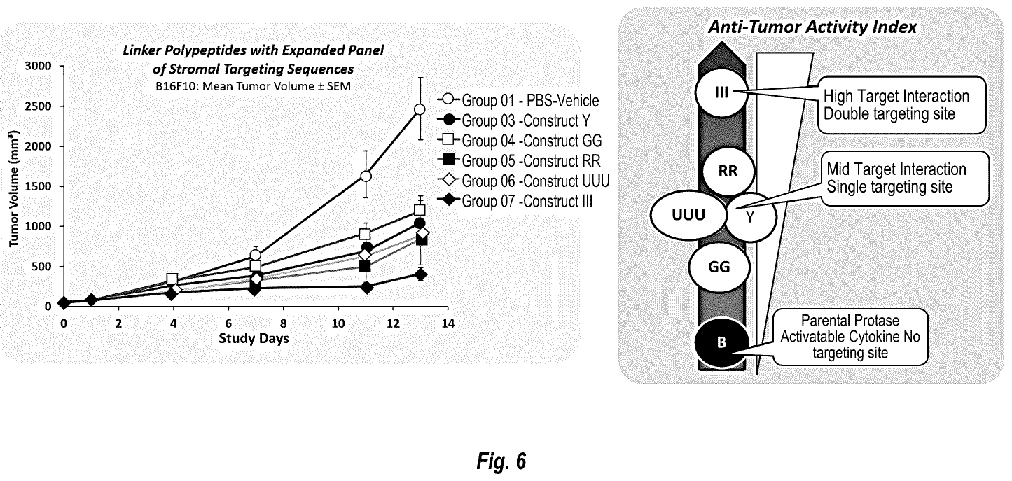

Tumor volumes were measured twice a week for the duration of the study. Mean tumor volume is shown in FIG. 6. Anti-tumor activity was observed in all treatment groups, but the most robust tumor growth inhibition (TGI) was observed with the multi-targeting linker construct Construct III (83.5%), compared to 52% to 66% TGI in single-targeting linker fusion proteins. On day 14, animals were sacrificed, and tissues and blood (processed to serum) were collected 24 hours post final dose (dose #5) and stored at −80° C. until further testing.

### Example 11: Multiple Targeting Sequences in Linker of IL-2 Fusion Protein LED to Increased Intratumoral Levels of Drug, IL-2, and IFN-γ, as Well as Enhanced Levels of Drug in Circulation Compared to Single-Targeting Linker Constructs

The levels of full-length IL-2-IL-2Ra fusion proteins, IL-2, and IFN-7 were quantified in tumor samples collected during a pre-clinical efficacy study comparing a panel of retention linker IL-2 fusion drugs (see Example 10).

Tumors (n=3 per group) were collected 24 hours after the last dose injection, flash frozen, and stored at −80° C. until further processing. Tumor lysates were generated using tissue extraction reagent (ThermoFisher) supplemented with protease and phosphatase inhibitors. Standard techniques and protein concentrations were determined using the BCA assay (Pierce).

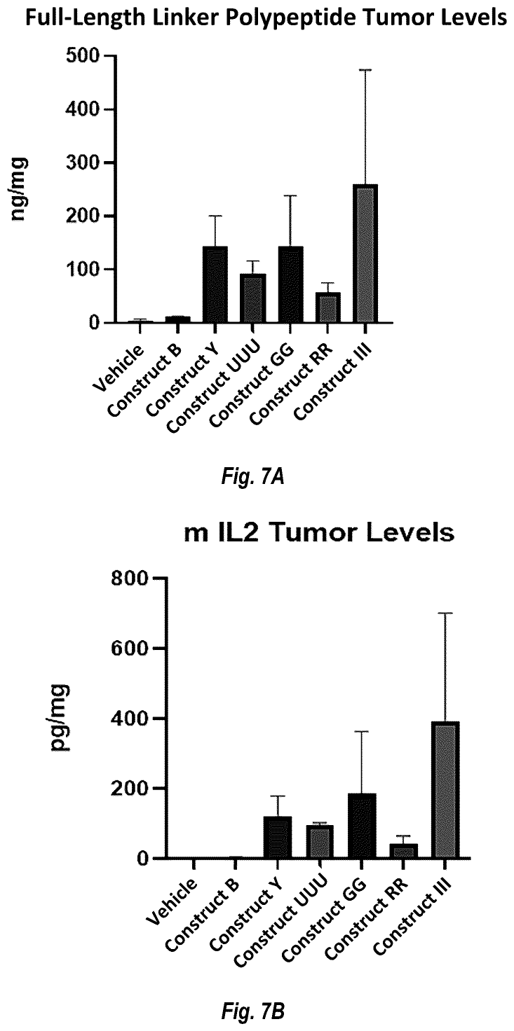

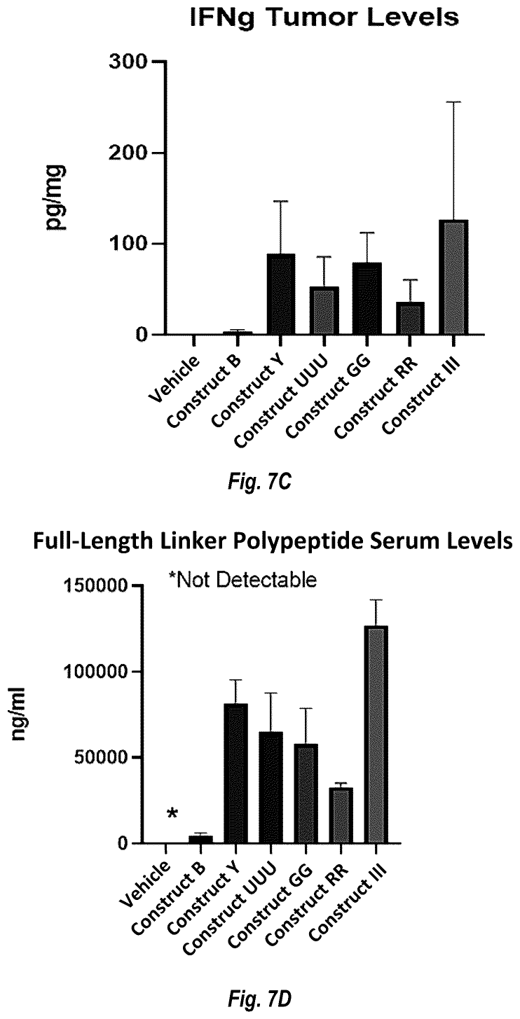

Lysates were tested with in-house developed ELISA (see Example 5) to measure full-length IL-2 fusion proteins (IL-2 capture/IL-2Ra detection). Results were normalized to 1 mg of tumor lysate and mean values are shown in FIG. 7A. The highest levels of drug were detected with the multi-targeting linker drug Construct III (about 2-fold to 5-fold higher levels compared to other retention linker drugs tested). Likewise, IL-2 intratumoral levels, measured with appropriate Luminex kit (IL-2 Mouse ProcartaPlex™ Simplex Kit, cat #EPX01A-20601-901, ThermoFisher), were highest in Construct III treated group compared to other arms (FIG. 7B). This demonstrates that multi-site targeting linker technology improved TME retention of both full-length drug and released active IL-2 post-cleavage. Moreover, levels of IFN-7, the main Th1 cytokine, were enhanced in Construct III animals (FIG. 7C; Essential Th1/Th2 Cytokine 6-Plex Mouse ProcartaPlex™ Panel, cat #EPX060-20831-901, ThermoFisher).

The equivalent serum samples (n=3 per group) were tested with in-house ELISA to quantify full-length IL-2 fusion drugs, and results are shown in FIG. 7D. 24 hours after dosing, circulating drug levels of Construct III are roughly 1.5-fold to 4-fold higher than other targeted drug serum levels. This demonstrates that engineering multiple targeting sequences into IL-2 fusion drugs increased drug levels in both tumor and circulation. Furthermore, multiple targeting sequences (e.g., a targeting sequence targeting heparin and a targeting sequence targeting collagen IV) can provide an increase in the serum half-life of the linker polypeptide.

### Example 12: Multiple Targeting Sequences in Linker of IL-2 Fusion Protein was not Associated with any Systemic Toxicity

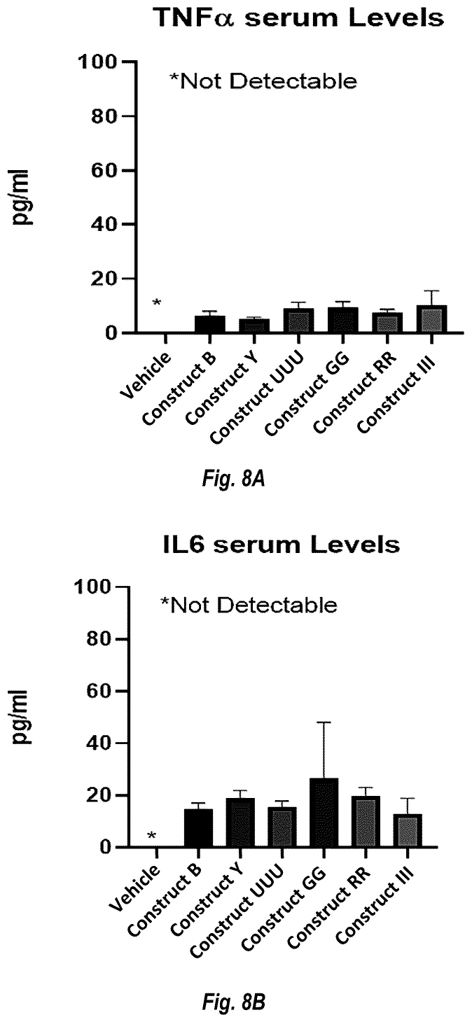

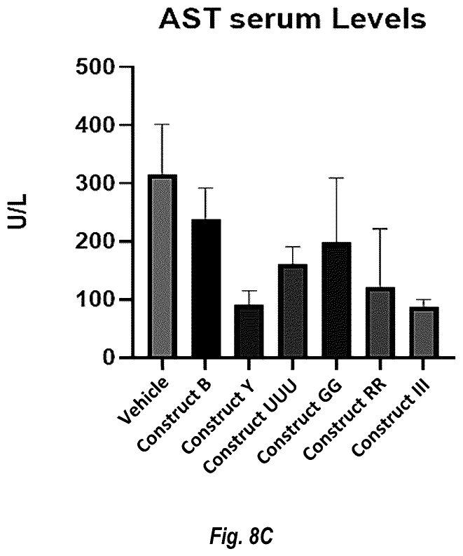

Inflammatory cytokine levels were measured in serum using a multiplex Luminex assay (Essential Th1/Th2 Cytokine 6-Plex Mouse ProcartaPlex™ Panel, cat #EPX060-20831-901, ThermoFisher). Low levels of TNF-α and IL-6 were detected (FIGS. 8A-8B; mean values per group equal or below 10 μg/mL and 27 μg/mL, respectively), while IL-12 was undetectable in all groups. In addition, no increase in aspartate transaminase levels was observed in treated arms compared to control animals, indicating the absence of any liver injury (FIG. 8C; AST activity assay, Sigma).

### Example 13: Linker Polypeptides with Immunoglobulin Antigen-Binding Domains as Active Domains

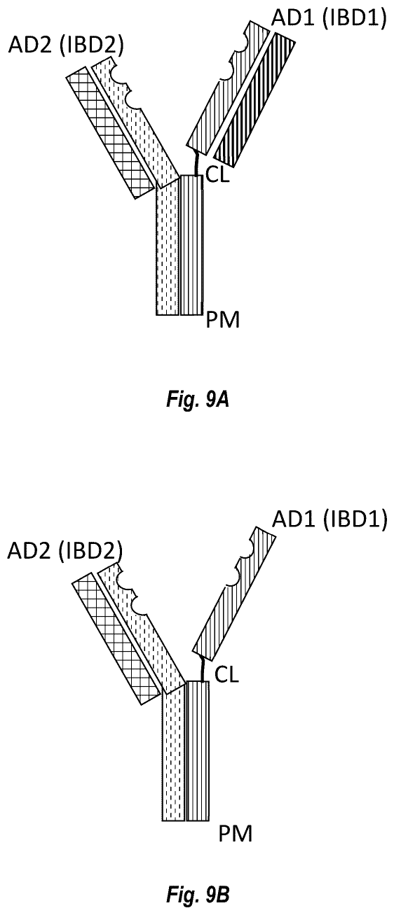

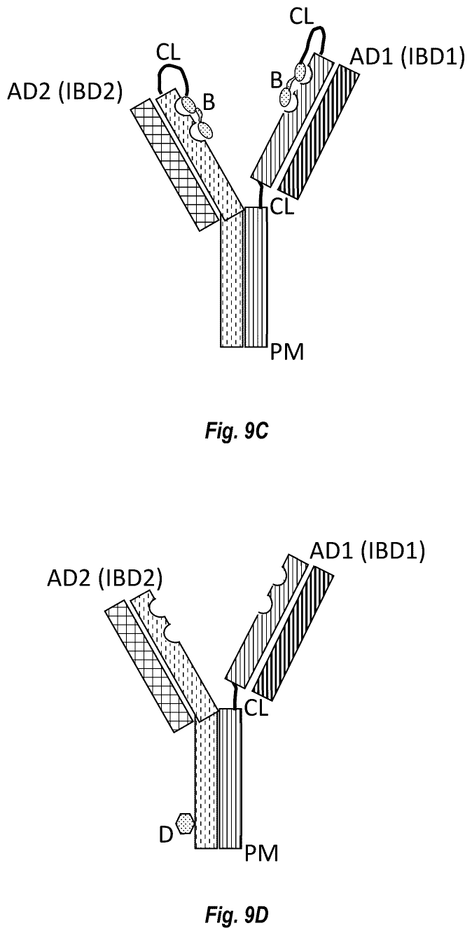

FIGS. 9A-9D each illustrate a linker polypeptide according to certain embodiments of the disclosure. The linker polypeptide of FIG. 9A comprises a first active domain (AD1); a second active domain (AD2); a pharmacokinetic modulator (PM); and a first linker between the pharmacokinetic modulator and the first active domain, the first linker comprising a protease-cleavable polypeptide sequence (CL). In some embodiments, the first linker further comprises a targeting sequence. In certain embodiments, the active domains comprise immunoglobulin antigen-binding domains (IBD1 and IBD2), which may be directed to different targets. In certain embodiments, the target binding domain may comprise a heavy chain and a light chain (FIG. 9A) or only a heavy chain (FIG. 9B), such as a VHH. Compared to the linker polypeptide of FIG. 9A, the linker polypeptide of FIG. 9D further comprises a chemotherapy drug (D).

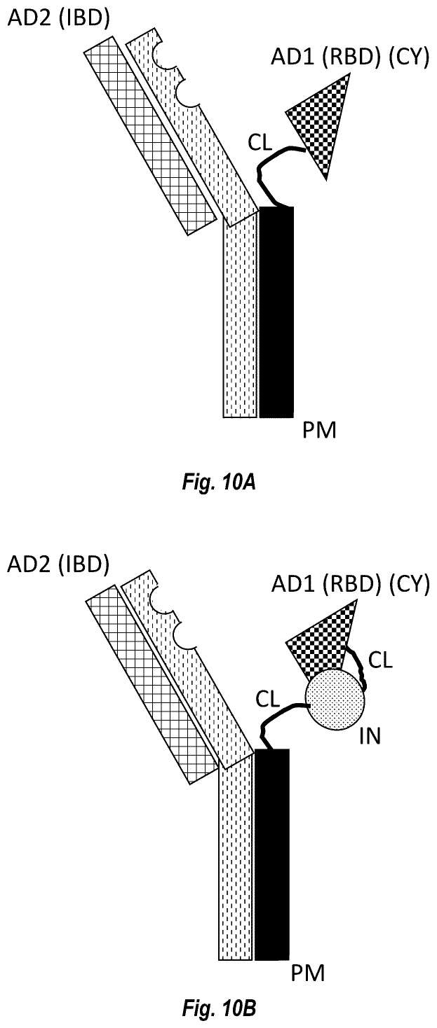

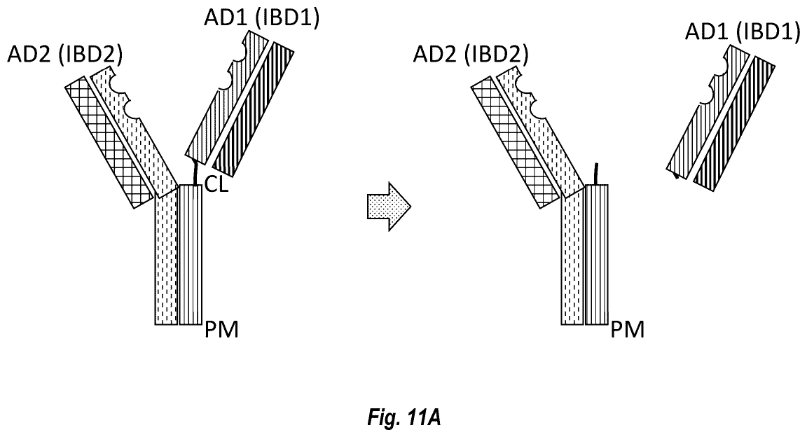

FIGS. 11A-11B each illustrate release of the first active domain from the remainder of the linker polypeptide after the one or more protease-cleavable polypeptide sequences are cleaved. In these figures, the active domains may comprise immunoglobulin antigen-binding domains (IBD1 and IBD2). Compared to the linker polypeptide of FIG. 11A, the linker polypeptide of FIG. 11B further comprises a blocker (B) conjugated, via a protease-cleavable polypeptide sequence (CL), to each of the first active domain and the second active domain. In some embodiments, the protease-cleavable polypeptide sequence connecting the first active domain to the remainder of the linker polypeptide and the protease-cleavable polypeptide sequences connecting the blockers to the active domains may be cleaved together (e.g., by the same protease). In some embodiments, the protease-cleavable polypeptide sequence connecting the first active domain to the remainder of the linker polypeptide and the protease-cleavable polypeptide sequences connecting the blockers to the active domains may be cleaved separately (e.g., by different proteases).

### Example 14: Linker Polypeptides with an Immunoglobulin Antigen-Binding Domain as One Active Domain and a Non-Immunoglobulin Polypeptide as the Other Active Domain

FIGS. 10A-10B each illustrates a linker polypeptide according to certain embodiments of the disclosure. The linker polypeptide of FIG. 10A comprises a first active domain (AD1); a second active domain (AD2); a pharmacokinetic modulator (PM); and a first linker between the pharmacokinetic modulator and the first active domain, the first linker comprising a protease-cleavable polypeptide sequence (CL). In some embodiments, the first linker further comprises a targeting sequence. In certain embodiments, the first active domain comprises a receptor-binding domain (RBD), and the second active domain comprises an immunoglobulin antigen-binding domain (IBD). In some embodiments, the RBD comprises a cytokine polypeptide sequence (CY). Compared to the linker polypeptide of FIG. 10A, the linker polypeptide of FIG. 10B further comprises an inhibitory polypeptide sequence (IN) capable of blocking an activity of the first active domain; and a second linker between the receptor-binding domain and the inhibitory polypeptide sequence, the second linker comprising a protease-cleavable polypeptide sequence (CL).

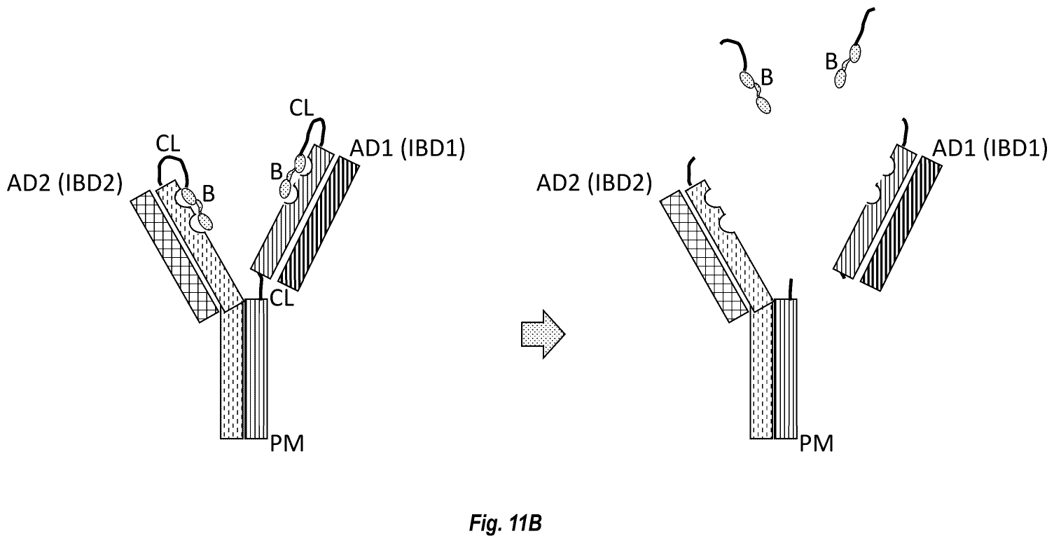

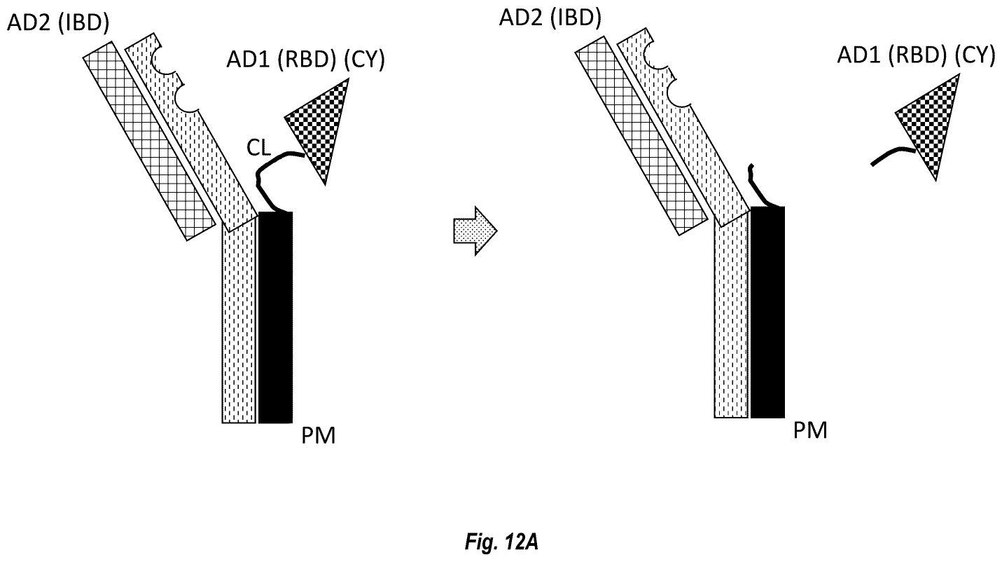

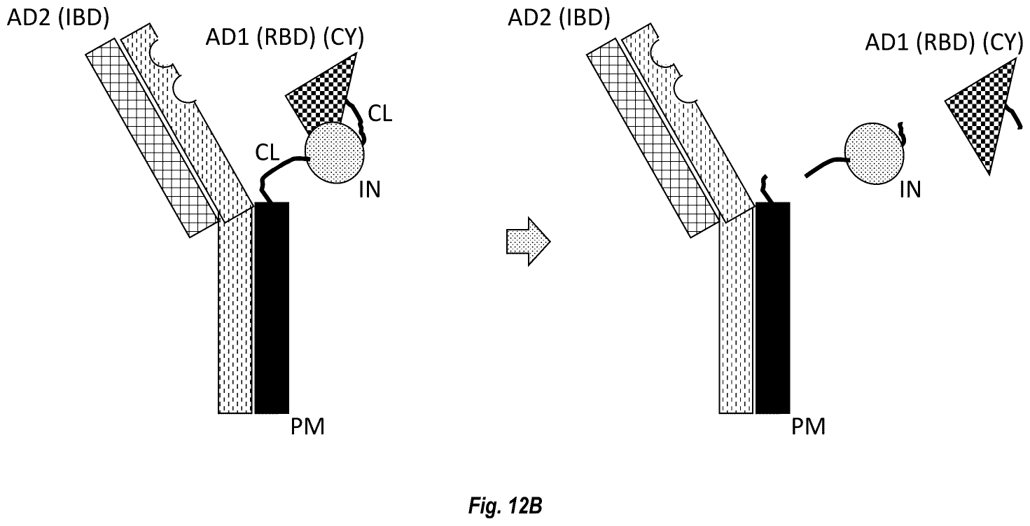

FIGS. 12A-12B each illustrate release of the first active domain from the remainder of the linker polypeptide after the one or more protease-cleavable polypeptide sequences are cleaved. In these figures, the first active domain comprises a receptor-binding domain (RBD), which may comprise a cytokine polypeptide sequence (CY), and the second active domain comprises an immunoglobulin antigen-binding domain (IBD). Compared to the linker polypeptide of FIG. 12A, the linker polypeptide of FIG. 12B further comprises an inhibitory polypeptide sequence (IN) capable of blocking an activity of the receptor-binding domain; and a second linker between the receptor-binding domain and the inhibitory polypeptide sequence, the second linker comprising a protease-cleavable polypeptide sequence (CL). In some embodiments, the protease-cleavable polypeptide sequences of the first linker and the second linker may be cleaved together (e.g., by the same protease). In some embodiments, the protease-cleavable polypeptide sequences of the first linker and the second linker may be cleaved separately (e.g., by different proteases).

### Example 15: Tumor Stroma Targeting Sequences in Linker of IL-2 Fusion Protein Yielded Enhanced Anti-Tumor Efficacy In Vivo

C57BL/6 mice were subcutaneously inoculated with MC38 colorectal cancer cells. When the average tumor volume reached 70-90 mm³, animals were randomized into 10 groups based on tumor volumes (n 7 or 6 mice per treatment group). Mice were dosed intraperitoneally (IP) twice-weekly (BIW) for a total of 5 doses according to design shown in Table 8 below:

TABLE 8
Dosing in C57BL/6 mice inoculated with MC38 cells

| | | | | Dosing | Dose | Dose |
|---|---|---|---|---|---|---|
| | | | Dose | Frequency & | Level | Volume |
| Group | Treatment | N | Route | Duration | (mg/kg) | (mL/kg) |
| 1 | PBS-Vehicle | 7 | IP | BIW for 14 days (5 doses | NA | 5 |
| | | | | D1, D4, D8, D11, D15) | | |
| 2 | Construct | 7 | IP | BIW for 14 days (5 doses | 5 | 5 |
| | AAA | | | D1, D4, D8, D11, D15) | | |
| 3 | Construct | 6 | IP | BIW for 14 days (5 doses | 1 | 5 |
| | AAA | | | D1, D4, D8, D11, D15) | | |
| 4 | Construct | 7 | IP | BIW for 14 days (5 doses | 5 | 5 |
| | EEE | | | D1, D4, D8, D11, D15) | | |
| 5 | Construct | 6 | IP | BIW for 14 days (5 doses | 1 | 5 |
| | EEE | | | D1, D4, D8, D11, D15) | | |
| 6 | Construct | 7 | IP | BIW for 14 days (5 doses | 5 | 5 |
| | NNN | | | D1, D4, D8, D11, D15) | | |
| 7 | Construct | 6 | IP | BIW for 14 days (5 doses | 1 | 5 |
| | NNN | | | D1, D4, D8, D11, D15) | | |
| 8 | Construct | 7 | IP | BIW for 14 days (5 doses | 5 | 5 |
| | NNNN | | | D1, D4, D8, D11, D15) | | |
| 9 | Construct | 6 | IP | BIW for 14 days (5 doses | 1 | 5 |
| | NNNN | | | D1, D4, D8, D11, D15) | | |
| 10 | Construct | 7 | IP | BIW for 14 days (5 doses | 5 | 5 |
| | OOOO | | | D1, D4, D8, D11, D15) | | |

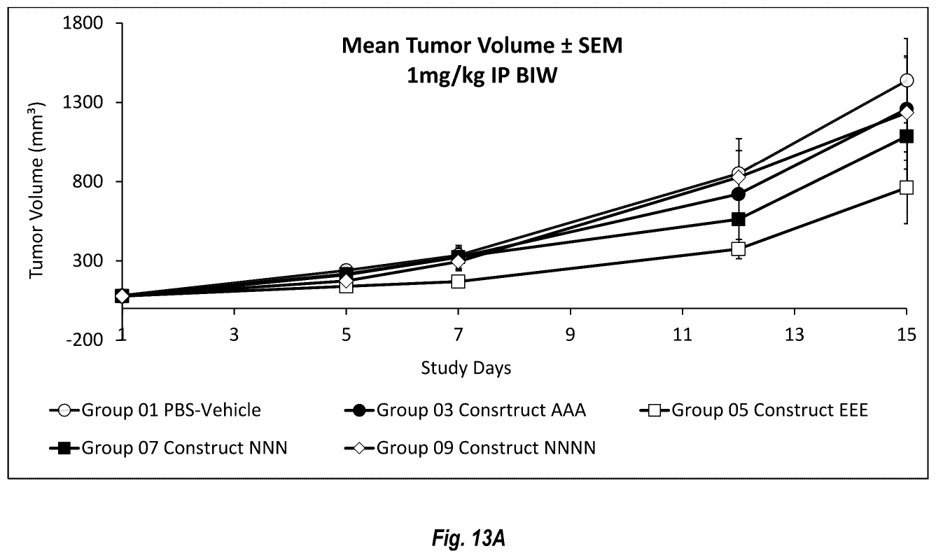

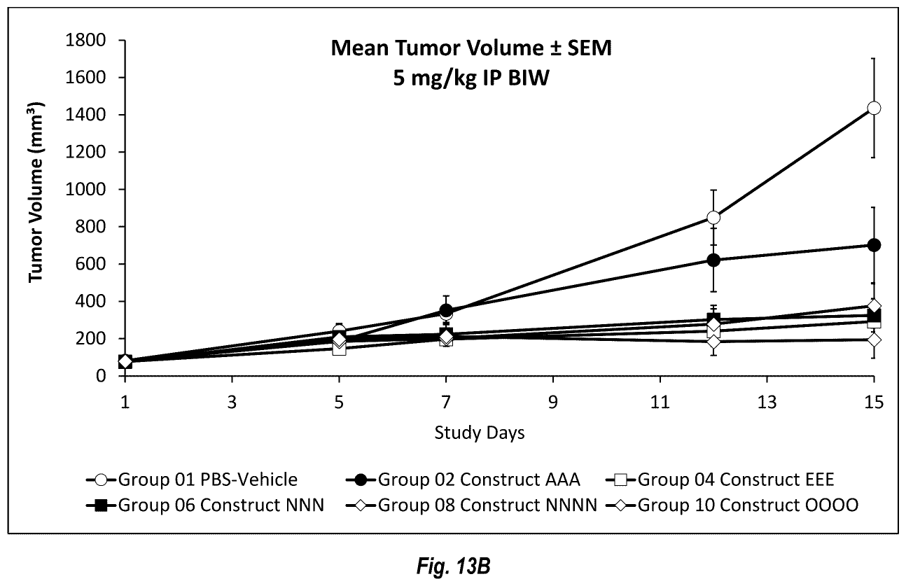

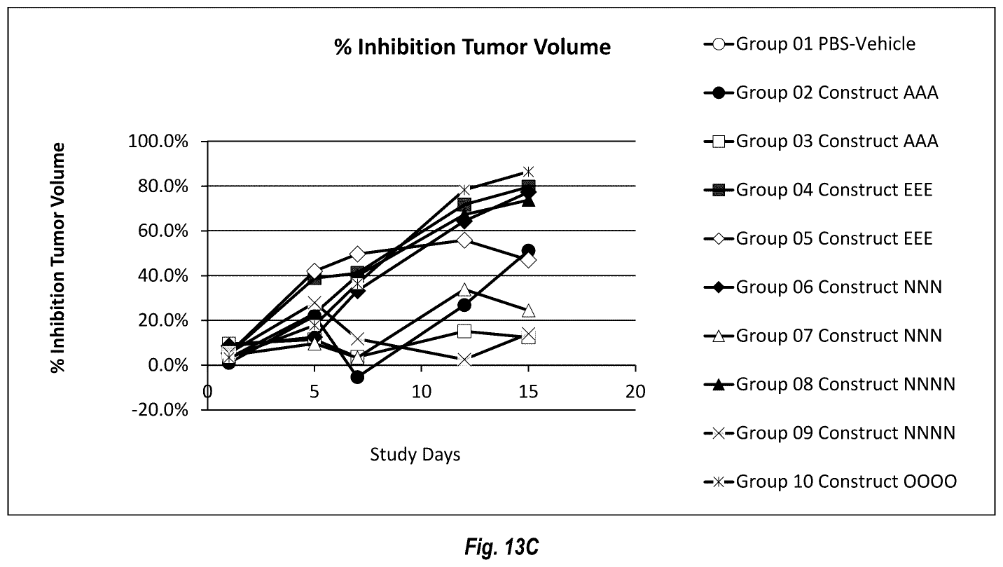

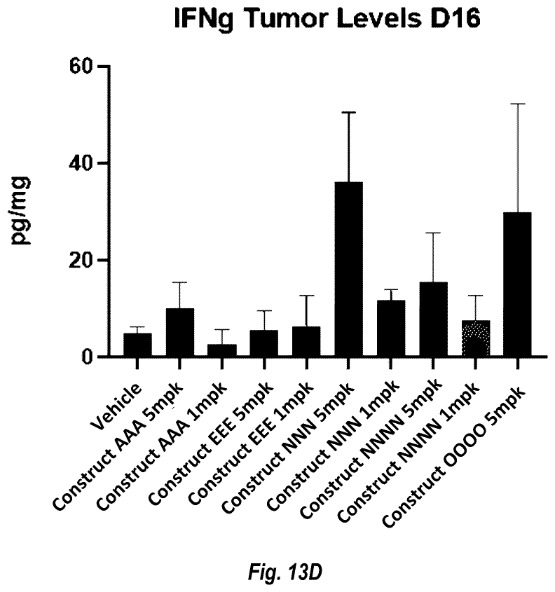

Tumor volumes were measured twice a week for the duration of the study. Mean tumor volume is shown in FIGS. 13A-13B, and inhibition of tumor volume is shown in FIG. 13C. Anti-tumor activity was observed in all treatment groups at the 5 mg/kg dose; however, the most robust tumor growth inhibition (TGI) was observed with the tumor-stroma-targeting Construct NNNN, Construct EEL, Construct NNN, and Construct 0000 (TGI ranging from 74% to 86%). More modest TGI was observed in low dose treatment groups, and tumor-stroma-targeting Construct EEE and Construct NNN continued to show superior efficacy over parental non-targeting constructs.

On Day 16, animals were sacrificed, and tumors (n=3 per group) were collected 24 hours after the last dose injection, flash frozen, and stored at −80° C. until further processing. Tumor lysates were generated using tissue extraction reagent (ThermoFisher) supplemented with protease and phosphatase inhibitors and standard techniques, and protein concentrations were determined using the BCA assay (Pierce). Intratumoral levels of IFN-7 (IFNg), the main Th1 cytokine, were mostly elevated in groups treated with targeting constructed, compared to groups treated with parental non-targeting constructs, as shown in FIG. 13D. IFN-7 was measured using Essential Th1/Th2 Cytokine 6-Plex Mouse ProcartaPlex™ Panel (cat #EPX060-20831-901, ThermoFisher).

### Example 16: IL-2 Fusion Proteins with TME Binding Motifs Showed Enhanced Intratumoral Immune Cell Infiltration

C57BL/6 mice were subcutaneously inoculated with B16F10 melanoma cells. When the average tumor volume reached 70-90 mm³, animals were randomized into 5 groups based on tumor volumes (n=3 mice per treatment group). Mice were dosed twice intraperitoneally on Day 1 and Day 4 with select ODC-IL2 fusions. On Day 6, tumors were harvested and processed into single cell suspension using standard technique (Miltenyi method, which is a combination of enzymatic and mechanical dissociation). Single cell samples were cryopreserved at −80° C. prior to further processing. Upon thawing, cells were washed and stained for surface and intracellular targets, using the antibodies listed in Table 9.

TABLE 9
Antibodies for staining immune cell markers

| Marker | Format | Clone | Catalog No. | Manufacturer |
|---|---|---|---|---|
| CD3 | AF700 | 17A2 | 100216 | Biolegend |
| CD4 | AF488 | GK1.5 | 100423 | Biolegend |
| CD8a | BV785 | 53-6.7 | 100750 | Biolegend |
| CD25 | PE-Cy7 | 3C7 | 101916 | Biolegend |
| DX5 | PCp/Cy5.5 | DX5 | 108916 | Biolegend |
| CD44 | BV650 | IM7 | 103049 | Biolegend |
| PD-1 | BV510 | 29F.1A12 | 135241 | Biolegend |
| CD45 | BV421 | 30-F11 | 103134 | Biolegend |
| Ki-67 | PE | 11F6 | 151210 | Biolegend |
| FoxP3 | APC | FJK-16s | 17-5773-82 | ThermoFisher |

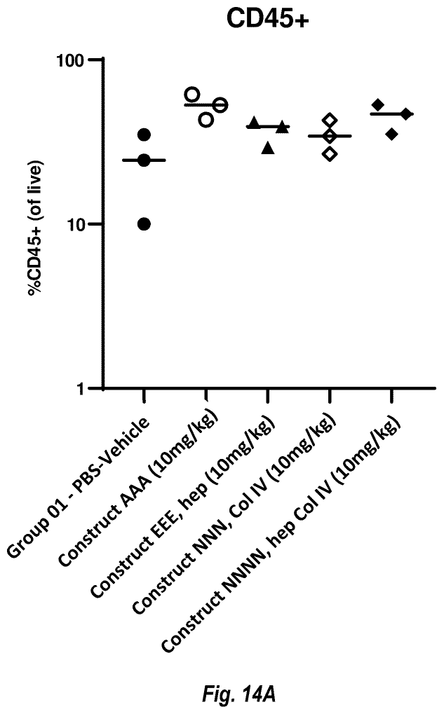

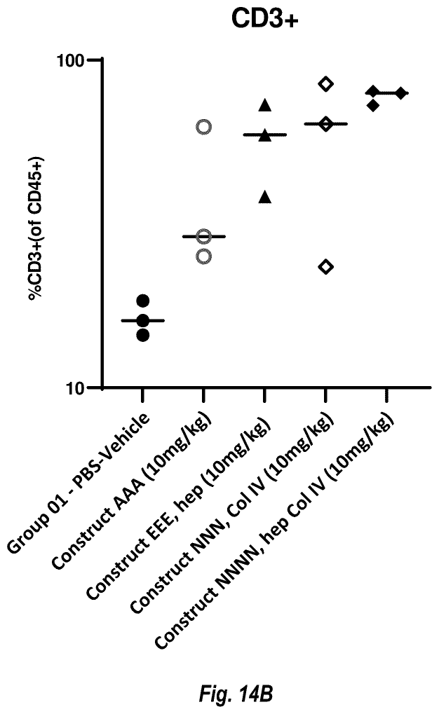

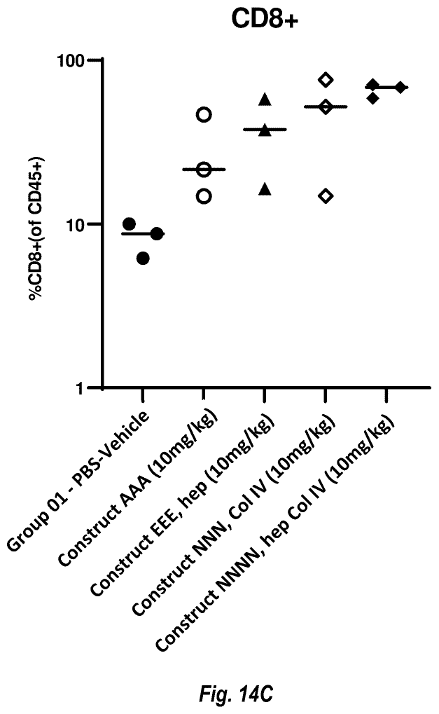

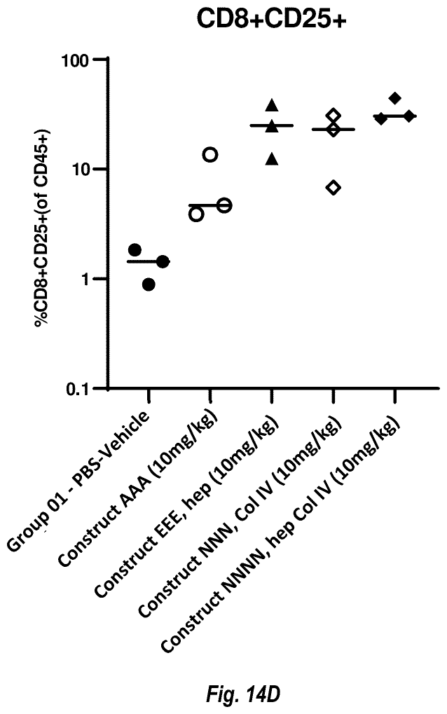

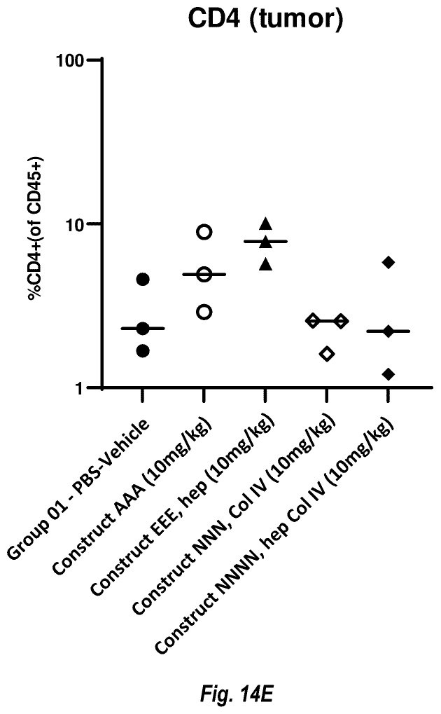

FIGS. 14A-14E show the flow cytometric analysis for select immune cell populations. Strikingly, groups treated with IL-2 fusion proteins engineered with tumor stroma targeting sites show enhanced intratumoral T cell infiltration (CD3+ cells), compared to groups treated with parental non-targeting fusion proteins or the vehicle group. More specifically, this T cell increase appeared to be driven primarily by an increase in both total and activated cytotoxic T cells (CD8+ and CD8+CD25+ subsets).

### Example 17: Examples of IL-2 Asymmetrical Fe Fusion Proteins with Tumor Targeting Sequences and Single or Dual Masks

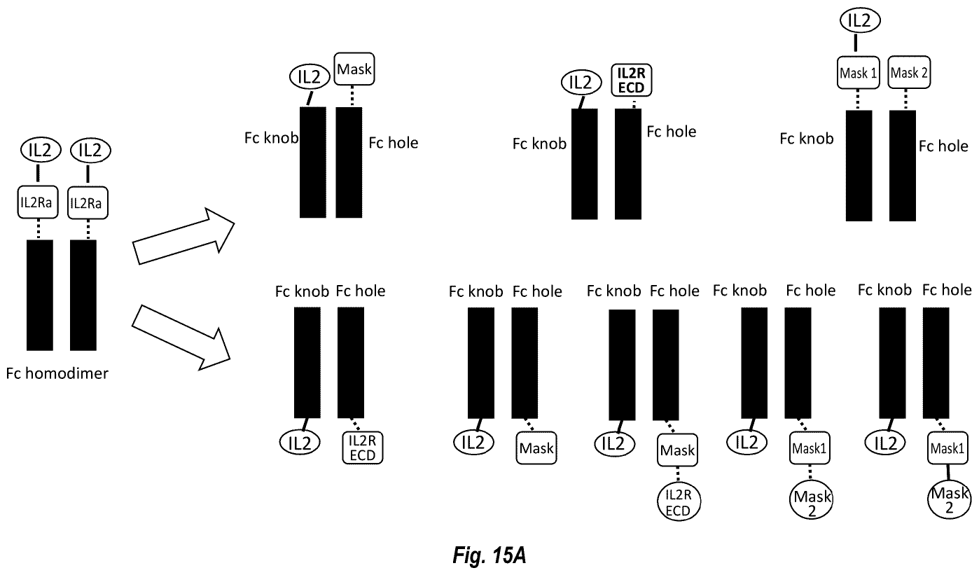

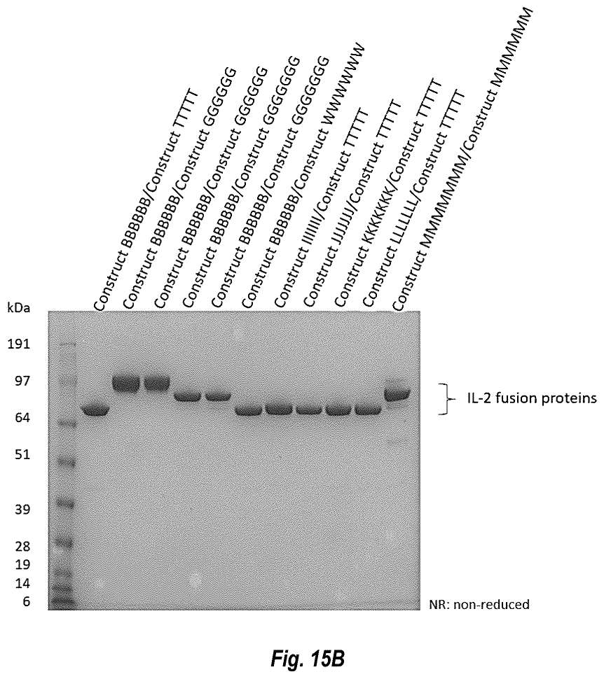

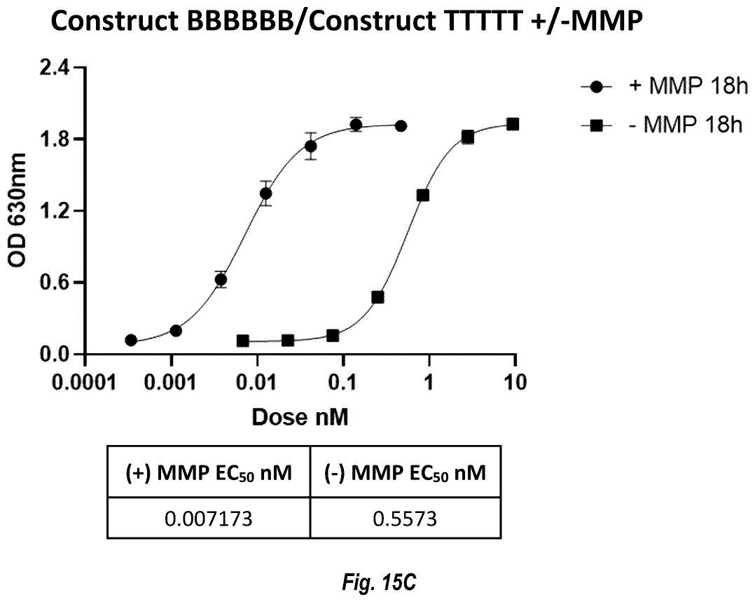

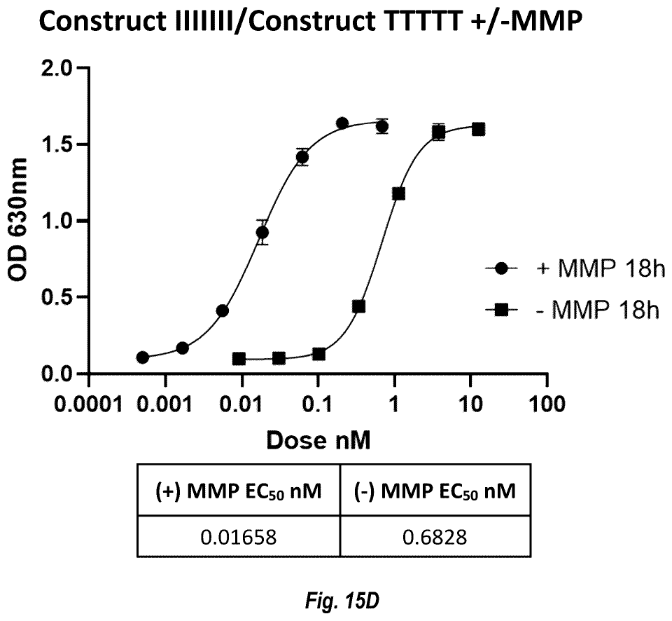

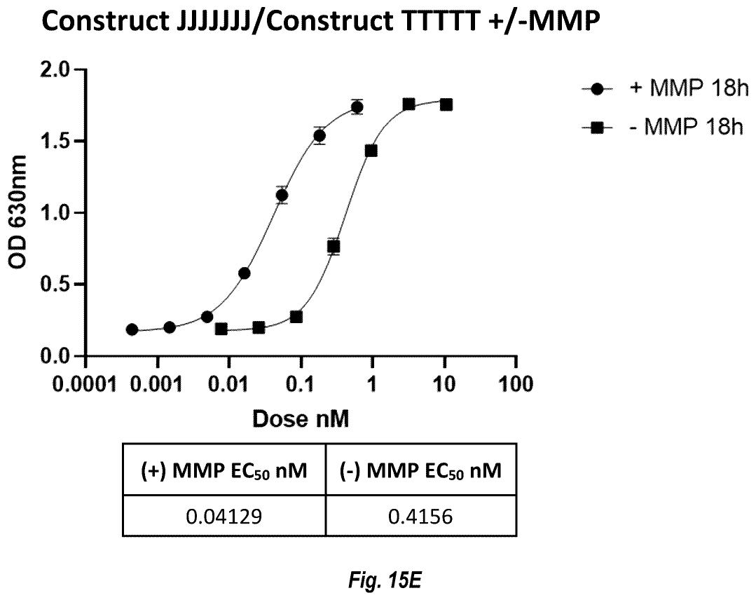

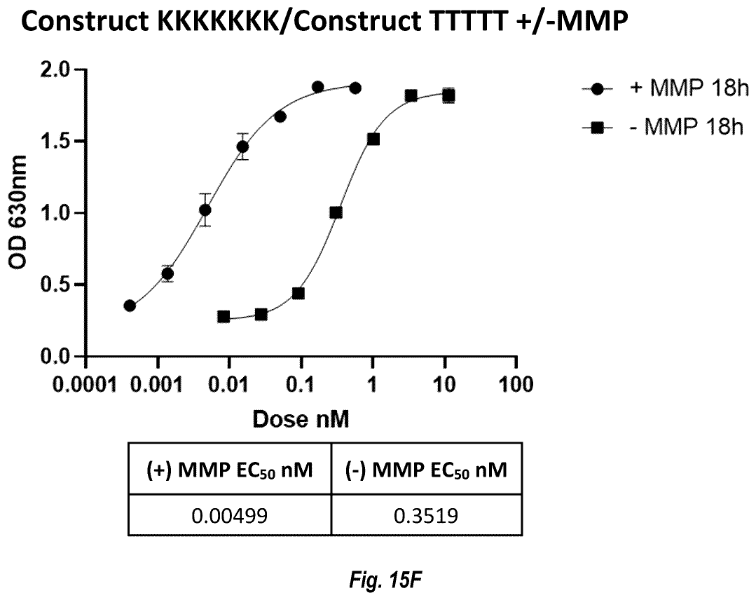

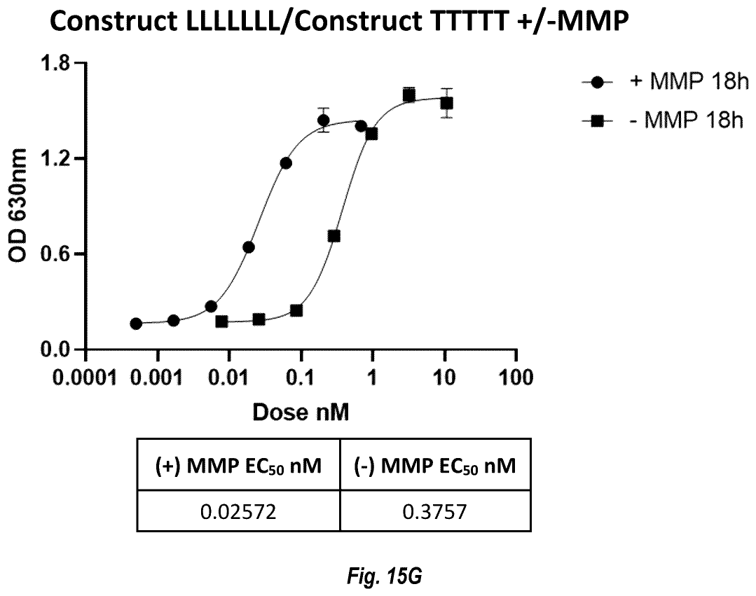

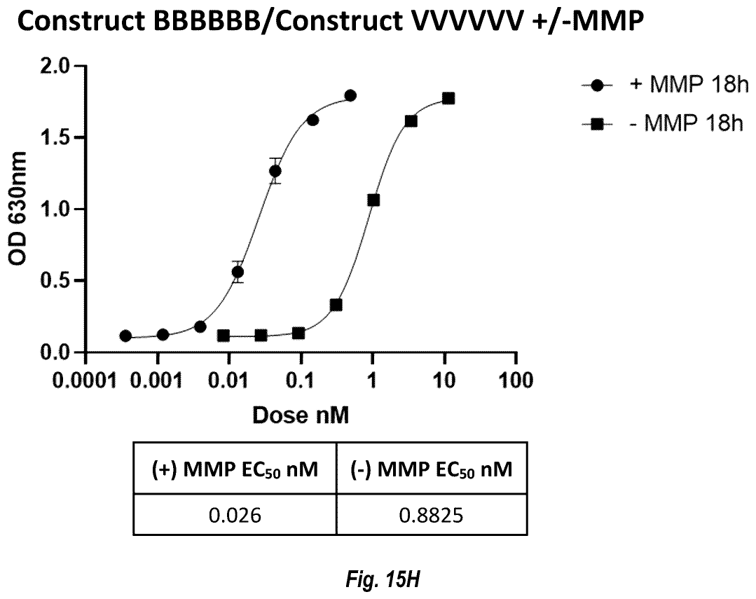

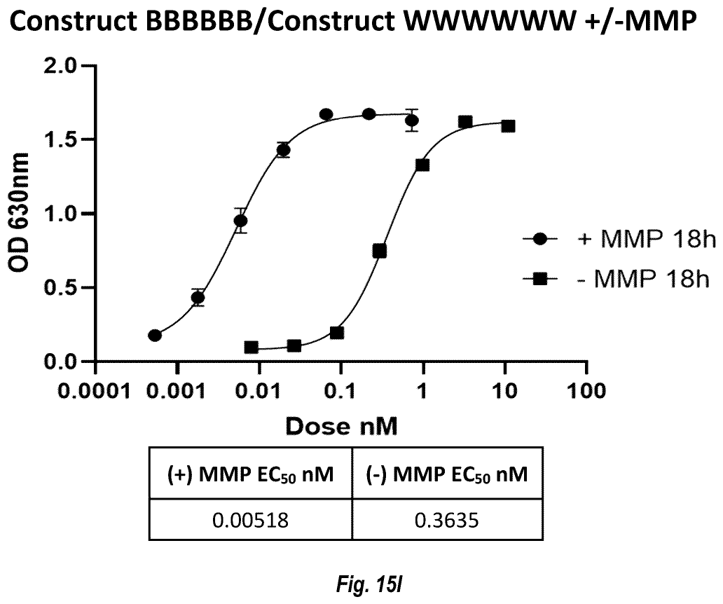

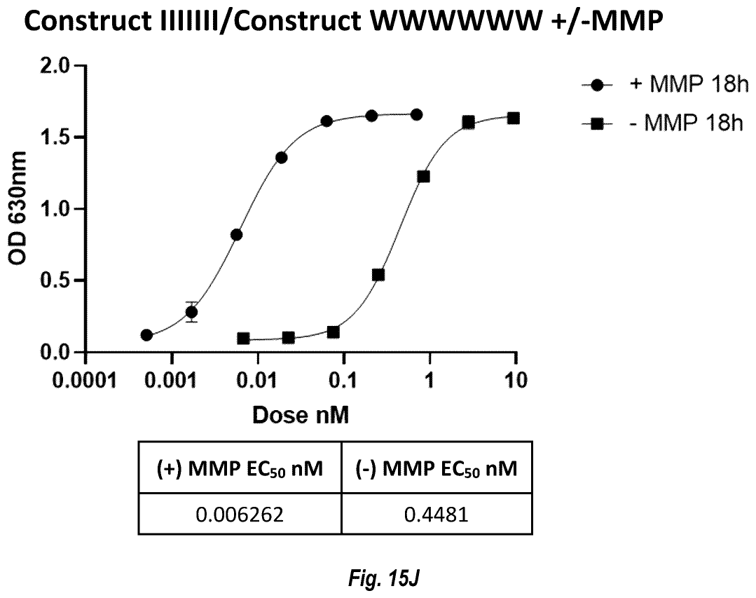

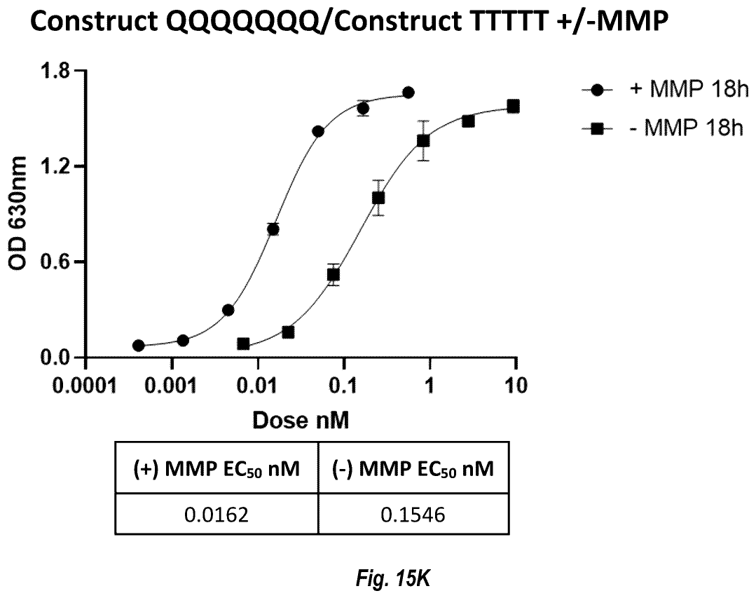

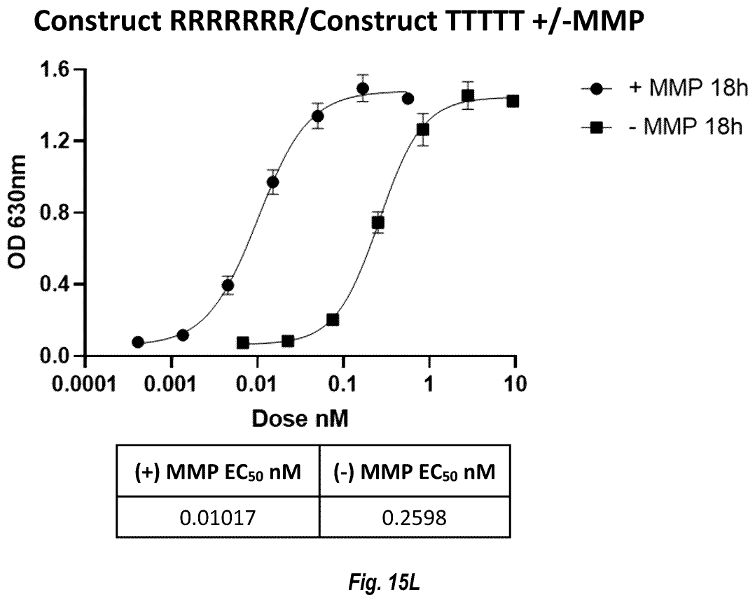

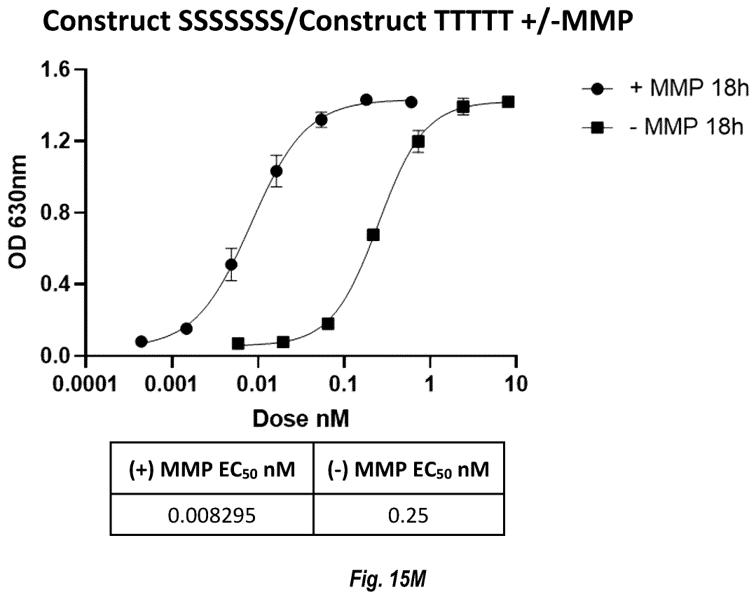

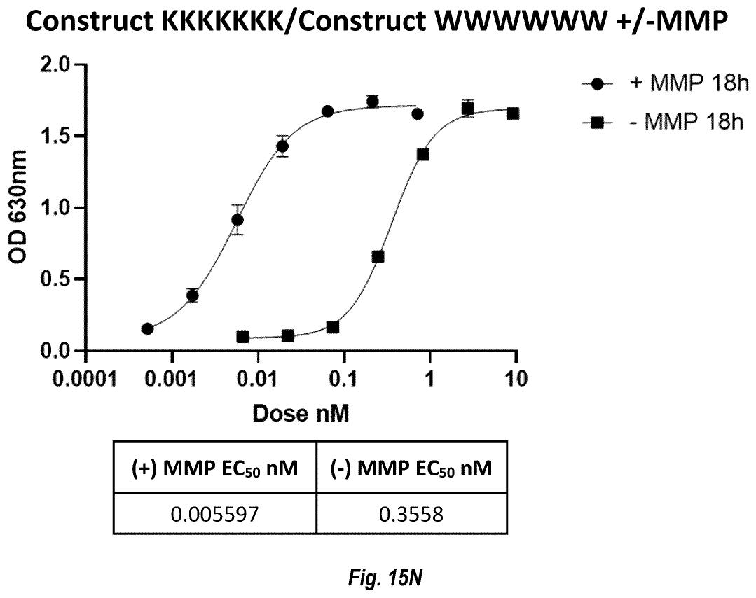

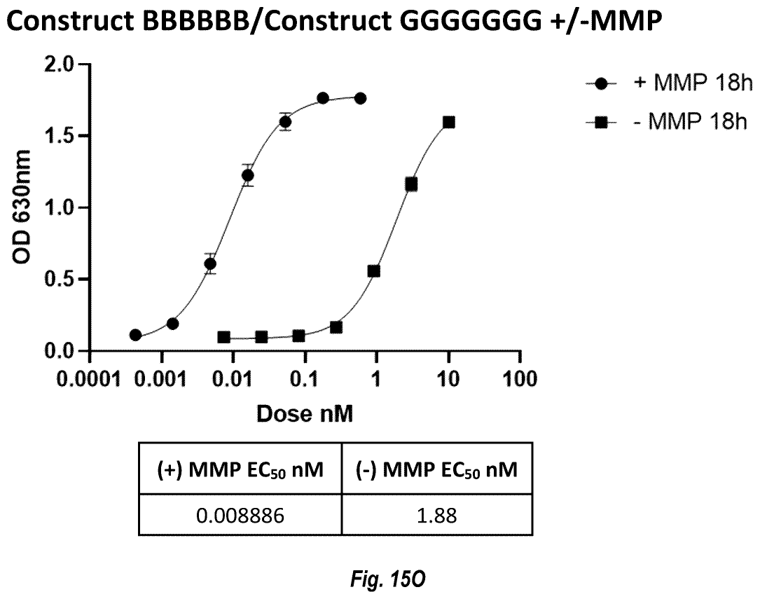

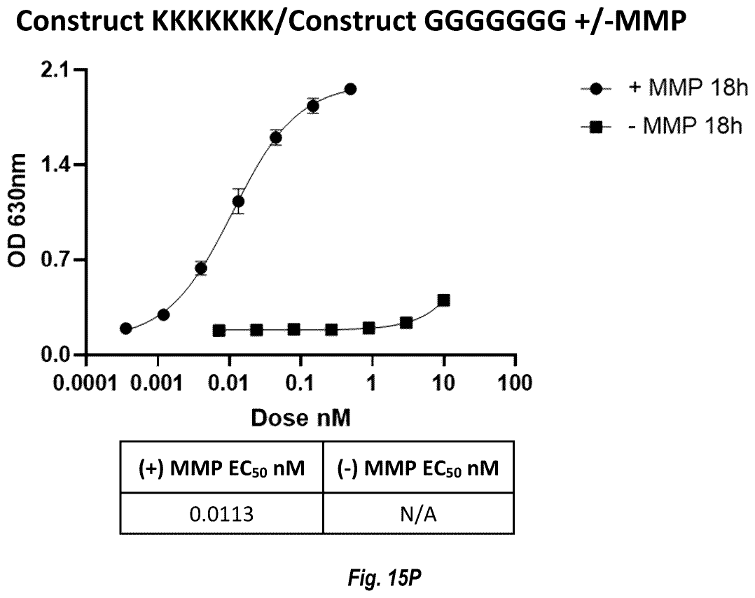

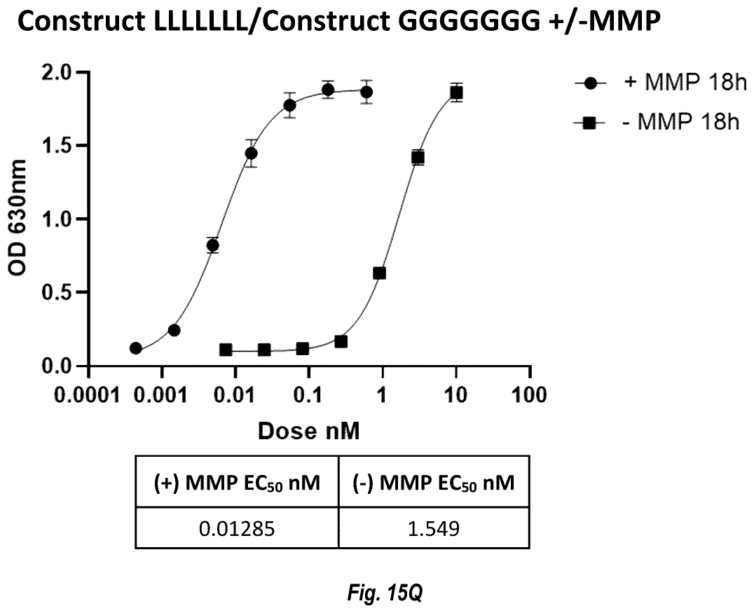

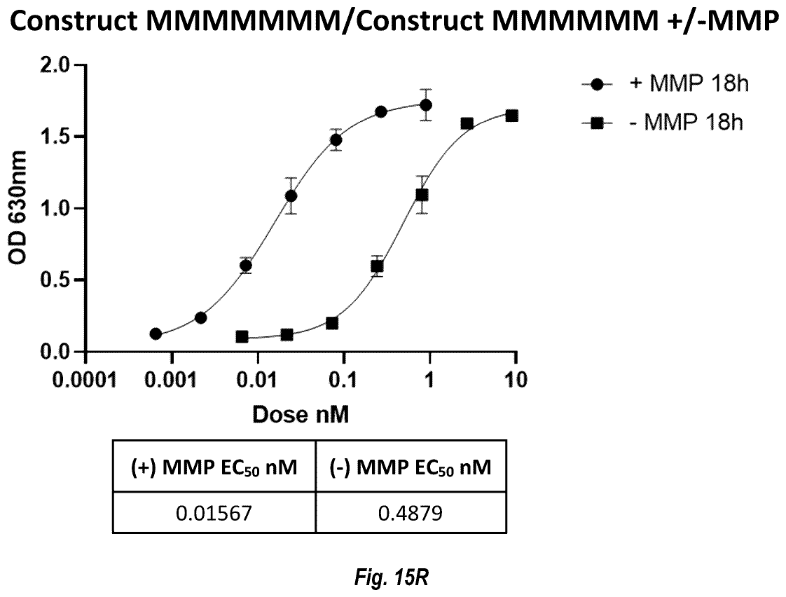

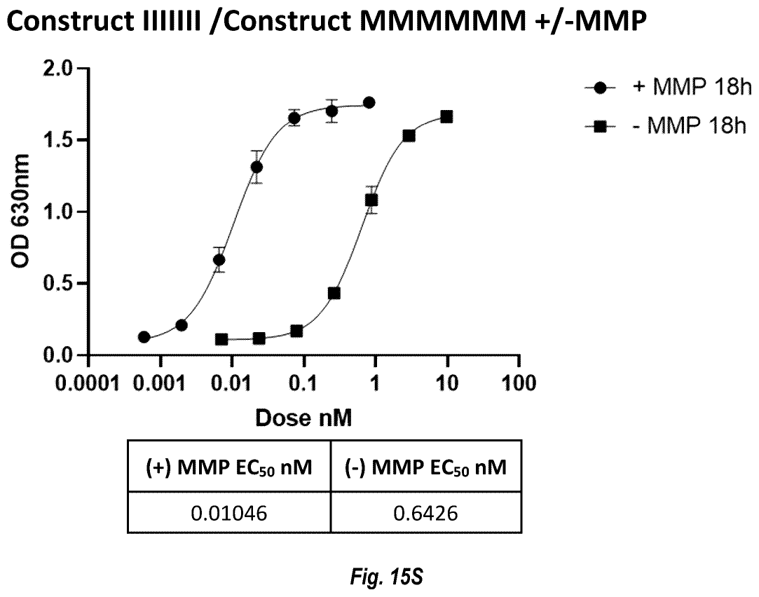

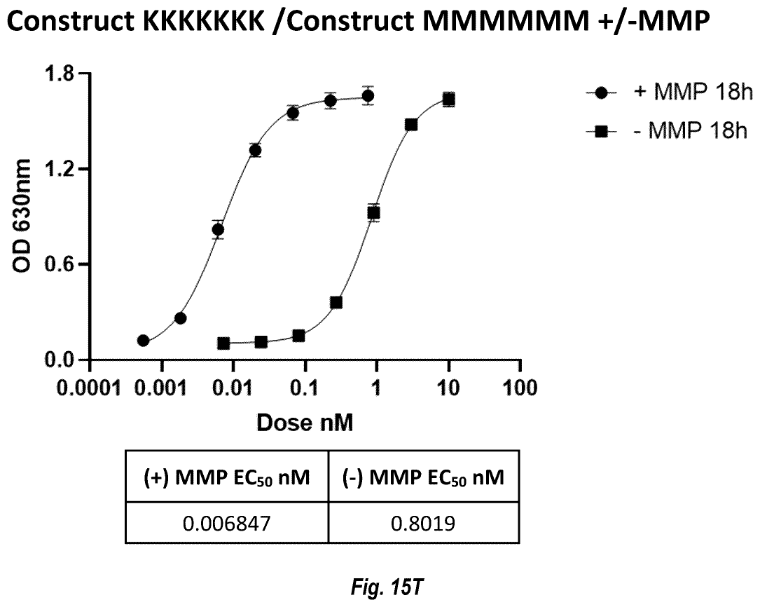

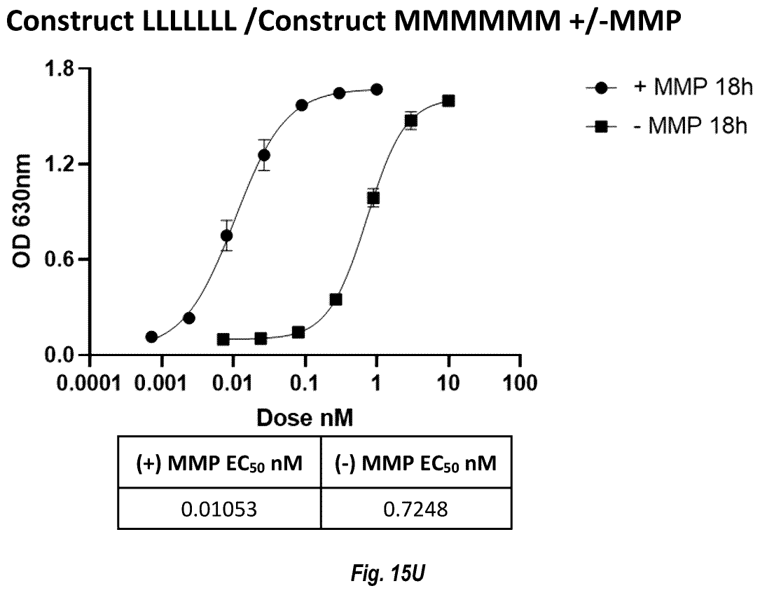

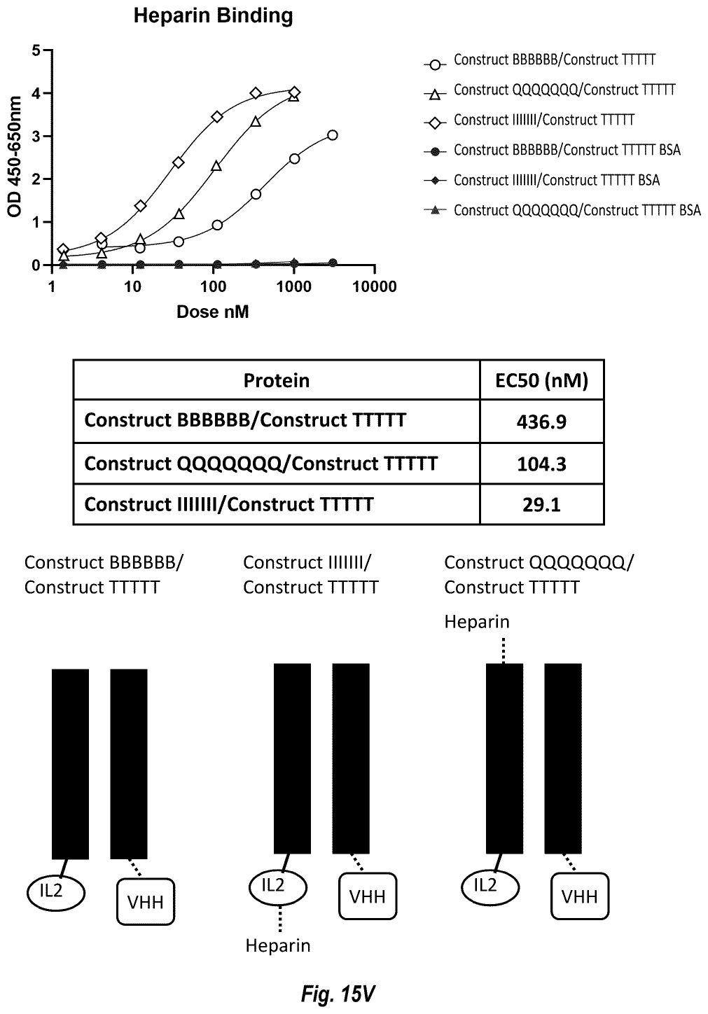

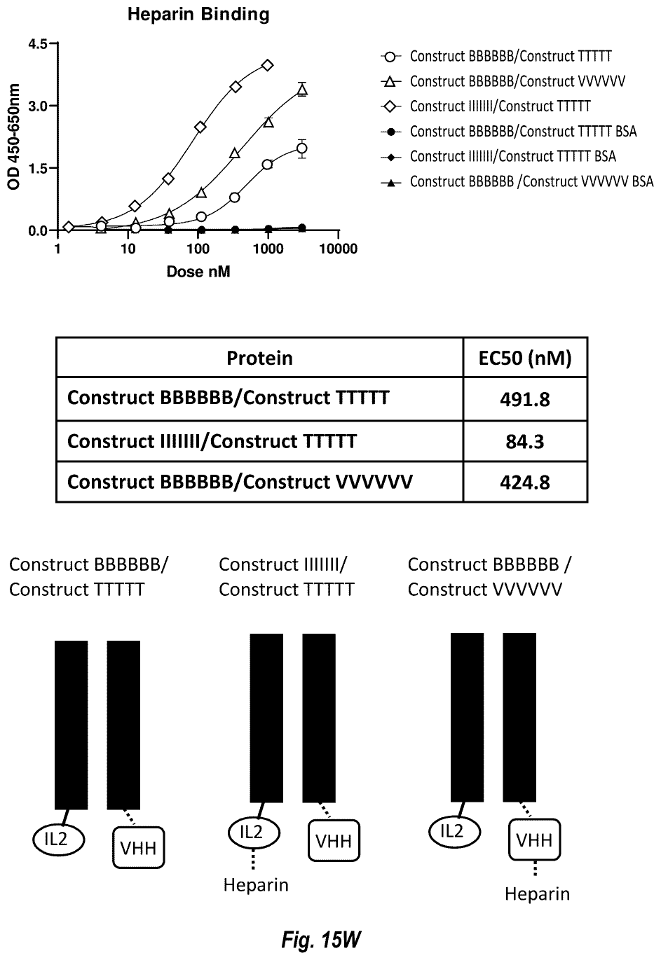

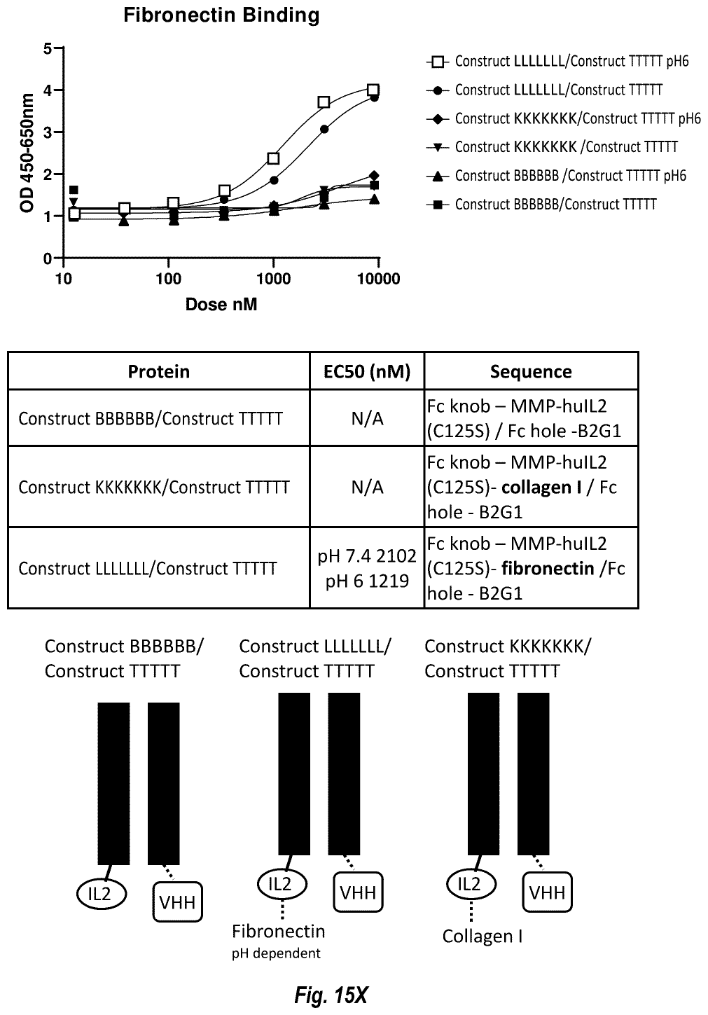

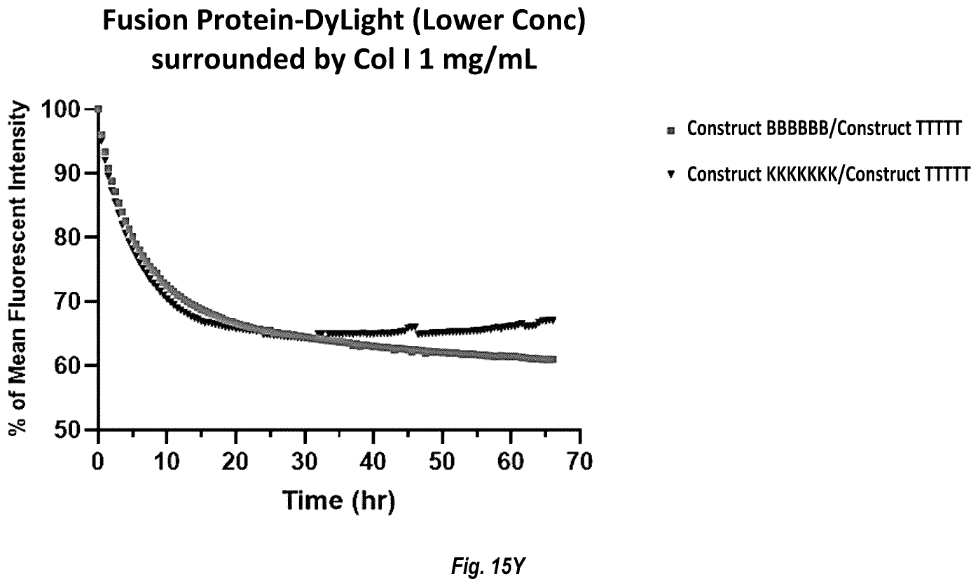

Additional asymmetrical IL-2 Fc fusion proteins containing ECM targeting sequences and single or dual masks were manufactured, purified, and functionally characterized as previously described. FIG. 15A shows examples of such proteins: the rectangles indicate Fc domains (either Fc knob or Fc hole), the solid lines indicate protease cleavable linker peptides, and the dashed lines indicate flexible linker sequences. The purity of Fc fusion proteins was assessed by SDS-PAGE under non-reducing conditions (FIG. 15B). Proteins were cleaved with recombinant MMP-9 protease overnight at 37° C., and digests were assessed in HEK-Blue IL-2 reporter assays as previously described. Results are shown in FIGS. 15C-15U. Select IL-2 fusion proteins were evaluated for their ability to bind ECM components such as heparin and fibronectin using the binding assays previously described, and results are shown in FIGS. 15V-15X. Fusion proteins with heparin binding motifs inserted at different locations of the molecule all showed enhanced binding to heparin compared to a parental molecule without tumor stroma targeting sites (FIGS. 15V-15W). Likewise, only an IL-2 fusion protein fusion engineered with a pH dependent fibronectin binding motif was able to bind fibronectin compared to a parental molecule without tumor stroma targeting sites or a fusion protein engineered with a collagen I binding motif (FIG. 15X). Furthermore, binding to fibronectin is slightly enhanced in acidic conditions.

In order to assess the ability of fusion proteins to bind collagen, an image-based retention assay was performed. Fusion proteins were labeled with DyLight 650 Maleimide at reduced sulfhydryl groups following manufacturer's recommended procedure (ThermoFisher, Cat #62295). Fluorescently labeled fusion proteins were then mixed with bovine type I collagen (Advanced Biomatrix, TeloCol-10, catalog #5226) and 10×PBS buffer, pH 7.4 (Invitrogen, REFAM9624) to bring the sample mix to a neutral pH. The final concentrations of each component in mix are shown in Table 10 below.

TABLE 10
Concentrations of components in fusion protein-collagen mix

| Component | Concentration |
|---|---|
| Construct BBBBBB/Construct TTTTT | 5.4 μM (right panel) |
| Construct KKKKKKK/Construct TTTTT | 3.4 μM (right panel) |
| Bovine type I collagen | 4 mg/ml |
| PBS | 1× |

5 μL of fusion protein-collagen mix was loaded to the inner well of ibidi u-Slide Angiogenesis (Uncoated, Part 81501) pretreated with gelatin solution (2% in H₂O, Sigma, Cat #G1393-20ML). The slide was incubated at room temperature for 30 minutes to allow the fusion protein-collagen mix to form gel. Then, 50 μL of bovine type I collagen (1 mg/mL in 1×PBS) was loaded to the upper well of the slide. After the collagen gelled in the upper well, the slide was imaged using a BioTek Lionheart FX automated microscope. The fluorescence intensity of the inner well represented the amount of fusion protein present and retained in the collagen and was measured at excitation/emission 628/685 nm. LED intensity, integration time, and camera gain were adjusted to appropriate levels to avoid excessive exposure and saturating pixel intensities. Fluorescence intensity was measured over 66 hours and images were taken every 30 minutes at room temperature. The mean fluorescence intensity was calculated by Gen5 software and then normalized to the mean fluorescence intensity of the first image (T=0), which was set to 100%. The normalized mean fluorescence intensity over time showed that the fusion protein containing a collagen I binding site is retained in collagen gel to a greater extent than a non-targeting fusion protein (FIG. 15Y).

1. A linker polypeptide, comprising:
a first targeting sequence;
a second targeting sequence; and
a first linker between the first targeting sequence and the second targeting sequence, the linker comprising a protease-cleavable polypeptide sequence.

2. The linker polypeptide of claim 1, wherein the linker polypeptide further comprises a first active domain, optionally wherein the first active domain is proximal to the first targeting sequence relative to the second targeting sequence,
optionally wherein the linker polypeptide further comprises an additional domain, optionally wherein the additional domain comprises an inhibitory polypeptide sequence capable of blocking an activity of the first active domain, a pharmacokinetic modulator, and/or a second active domain, and optionally wherein the additional domain is proximal to the second targeting sequence relative to the first targeting sequence, and
optionally wherein the linker polypeptide comprises sequentially, from the N-terminus to the C-terminus or from the C-terminus to the N-terminus, the first active domain, the first targeting sequence, the first linker, the second targeting sequence, and the additional domain.

3-4. (canceled)

5. A linker polypeptide, comprising
a first active domain;
a second active domain;
a pharmacokinetic modulator; and
a first linker between the pharmacokinetic modulator and the first active domain, the first linker comprising a protease-cleavable polypeptide sequence; and
optionally a first targeting sequence.

6. (canceled)

7. A linker polypeptide, comprising:
a first active domain;
an inhibitory polypeptide sequence capable of blocking an activity of the first active domain;
a first linker between the first active domain and the inhibitory polypeptide sequence, the linker comprising a protease-cleavable polypeptide sequence; and
a first targeting sequence; and
optionally a pharmacokinetic modulator.

8. (canceled)

9. A linker polypeptide, comprising:
a first polypeptide chain comprising a first active domain, a first domain of a pharmacokinetic modulator, and a first linker between the first active domain and the first domain of the pharmacokinetic modulator, wherein the first active domain is C-terminal to or N-terminal to the first domain of the pharmacokinetic modulator;
a second polypeptide chain, comprising a second domain of the pharmacokinetic modulator, an inhibitory polypeptide sequence capable of blocking an activity of the first active domain, and a second linker between the second domain of the pharmacokinetic modulator and the inhibitory polypeptide sequence;
wherein the first linker comprises a protease-cleavable polypeptide sequence; and
the first polypeptide chain or the second polypeptide chain further comprises at least one targeting sequence.

10. (canceled)

11. The linker polypeptide of claim 9, wherein the inhibitory polypeptide sequence is C-terminal to the second domain of the pharmacokinetic modulator, or wherein the inhibitory polypeptide sequence is N-terminal to the second domain of the pharmacokinetic modulator; and/or
wherein the targeting sequence is between the protease-cleavable polypeptide sequence and the first domain of the pharmacokinetic modulator, or wherein the targeting sequence is between the protease-cleavable polypeptide sequence and the first active domain, or wherein the targeting sequence is C-terminal to the first active domain, or wherein the targeting sequence is N-terminal to the first active domain, or wherein the targeting sequence is C-terminal to the inhibitory polypeptide sequence, or wherein the targeting sequence is N-terminal to the inhibitory polypeptide sequence, or wherein the targeting sequence is between the inhibitory polypeptide sequence and the second domain of the pharmacokinetic modulator.

12-19. (canceled)

20. The linker polypeptide of claim 9, wherein the targeting sequence binds heparin, optionally wherein the targeting sequence comprises SEQ ID NO: 664; or
wherein the targeting sequence binds collagen IV, optionally wherein the targeting sequence comprises SEQ ID NO: 200: or
wherein the targeting sequence binds collagen I, optionally wherein the targeting sequence comprises SEQ ID NO: 188: or
wherein the targeting sequence binds fibronectin, optionally wherein the targeting sequence comprises SEQ ID NO: 653.

21-23. (canceled)

24. The linker polypeptide of claim 9, wherein the targeting sequence is a first targeting sequence and the linker polypeptide further comprises a second targeting sequence, optionally wherein the first targeting sequence is part of the first polypeptide chain and the second targeting sequence is part of the second polypeptide chain, and optionally wherein the first targeting sequence is C-terminal to the first active domain and the second targeting sequence is C-terminal to the inhibitory polypeptide sequence.

25-26. (canceled)

27. The linker polypeptide of claim 24, wherein the second targeting sequence binds heparin, optionally wherein the targeting sequence comprises SEQ ID NO: 664; or
wherein the second targeting sequence binds collagen IV, optionally wherein the targeting sequence comprises SEQ ID NO: 200; or
wherein the second targeting sequence binds collagen I, optionally wherein the targeting sequence comprises SEQ ID NO: 188; or
wherein the second targeting sequence binds fibronectin, optionally wherein the targeting sequence comprises SEQ ID NO: 653.

28-30. (canceled)

31. The linker polypeptide of claim 9, further comprising a second active domain, optionally wherein the second active domain is part of the second polypeptide chain.

32. The linker polypeptide of claim 9, wherein the inhibitory polypeptide sequence is a first inhibitory polypeptide sequence, and the linker polypeptide further comprises a second inhibitory polypeptide sequence, optionally wherein the second inhibitory polypeptide sequence is part of the second polypeptide chain, and optionally wherein the second inhibitory polypeptide sequence is C-terminal to the first inhibitory polypeptide sequence; and optionally
wherein the second inhibitory polypeptide sequence is an immunoglobulin inhibitory polypeptide sequence, optionally wherein the first inhibitory polypeptide sequence is an immunoglobulin inhibitory polypeptide sequence, and optionally wherein one or each of the immunoglobulin inhibitory polypeptide sequences is a VHH.

33-37. (canceled)

38. The linker polypeptide of claim 9, wherein the pharmacokinetic modulator comprises a heterodimeric Fc or heterodimeric CH3 domains, optionally wherein the heterodimeric Fc or heterodimeric CH3 domains comprise a knob CH3 domain and a hole CH3 domain, and optionally wherein the first domain of the pharmacokinetic modulator is a knob CH3 domain and the second domain of the pharmacokinetic modulator is a hole CH3 domain, or the first domain of the pharmacokinetic modulator is a hole CH3 domain and the second domain of the pharmacokinetic modulator is a knob CH3 domain; and optionally
wherein the pharmacokinetic modulator comprises the sequence of SEQ ID NO: 76, 75, 77, 78, 756, or 757.

39-47. (canceled)

48. The linker polypeptide of claim 9, wherein the first active domain comprises a first immunoglobulin antigen-binding domain and/or wherein the second active domain comprises a second immunoglobulin antigen-binding domain; and/or
wherein one or each of the first immunoglobulin antigen-binding domain and the second immunoglobulin antigen-binding domain independently comprises a VH region and a VL region; and/or
wherein one or each of the first immunoglobulin antigen-binding domain and the second immunoglobulin antigen-binding domain independently comprises an Fv, scFv, Fab, or VHH; and/or
wherein one or each of the first immunoglobulin antigen-binding domain and the second immunoglobulin antigen-binding domain independently is humanized or fully human; and/or
wherein one or each of the first immunoglobulin antigen-binding domain and the second immunoglobulin antigen-binding domain independently is configured to bind to one or more sequences selected from a cancer cell surface antigen sequence, a growth factor sequence, and a growth factor receptor sequence, optionally wherein one or each of the first immunoglobulin antigen-binding domain and the second immunoglobulin antigen-binding domain independently is configured to bind to a HER2 sequence, an EGFR extracellular domain sequence, a PD-1 extracellular domain sequence, a PD-L1 extracellular domain sequence, or a CD3 extracellular domain sequence; and/or
wherein one of the first immunoglobulin antigen-binding domain and the second immunoglobulin antigen-binding domain is configured to bind to a HER2 sequence, optionally wherein one of the first immunoglobulin antigen-binding domain and the second immunoglobulin antigen-binding domain comprises a VH region comprising hypervariable regions (HVRs) HVR-1, HVR-2, and HVR-3 of a VH region comprising the amino acid sequence of SEQ ID NO: 910, and a VL region comprising HVR-1, HVR-2, and HVR-3 of a VL region comprising the amino acid sequence of SEQ ID NO: 909, optionally wherein one of the first immunoglobulin antigen-binding domain and the second immunoglobulin antigen-binding domain comprises a sequence that has at least 80, 85, 90, 95, 97, 98, or 99 percent identity to the sequence of SEQ ID NO: 909 or 910, optionally wherein one of the first immunoglobulin antigen-binding domain and the second immunoglobulin antigen-binding domain comprises a VH region comprising the amino acid sequence of SEQ ID NO: 910; and a VL region comprising the amino acid sequence of SEQ ID NO: 909, and optionally wherein one of the first immunoglobulin antigen-binding domain and the second immunoglobulin antigen-binding domain is an antigen-binding domain of trastuzumab; and/or
wherein one of the first immunoglobulin antigen-binding domain and the second immunoglobulin antigen-binding domain is configured to bind to an EGFR extracellular domain sequence, optionally wherein one of the first immunoglobulin antigen-binding domain and the second immunoglobulin antigen-binding domain comprises a VH region comprising HVR-1, HVR-2, and HVR-3 of a VH region comprising the amino acid sequence of SEQ ID NO: 914, and a VL region comprising HVR-1, HVR-2, and HVR-3 of a VL region comprising the amino acid sequence of SEQ ID NO: 913, optionally wherein one of the first immunoglobulin antigen-binding domain and the second immunoglobulin antigen-binding domain comprises a sequence that has at least 80, 85, 90, 95, 97, 98, or 99 percent identity to the sequence of SEQ ID NO: 913 or 914, optionally wherein one of the first immunoglobulin antigen-binding domain and the second immunoglobulin antigen-binding domain comprises a VH region comprising the amino acid sequence of SEQ ID NO: 914; and a VL region comprising the amino acid sequence of SEQ ID NO: 913, and optionally wherein one of the first immunoglobulin antigen-binding domain and the second immunoglobulin antigen-binding domain is an antigen-binding domain of cetuximab; and/or
wherein one of the first immunoglobulin antigen-binding domain and the second immunoglobulin antigen-binding domain is configured to bind to a PD-1 extracellular domain sequence, optionally wherein one of the first immunoglobulin antigen-binding domain and the second immunoglobulin antigen-binding domain comprises a VH region comprising HVR-1, HVR-2, and HVR-3 of a VH region comprising the amino acid sequence of SEQ ID NO: 917, and a VL region comprising HVR-1, HVR-2, and HVR-3 of a VL region comprising the amino acid sequence of SEQ ID NO: 918, optionally wherein one of the first immunoglobulin antigen-binding domain and the second immunoglobulin antigen-binding domain comprises a sequence that has at least 80, 85, 90, 95, 97, 98, or 99 percent identity to the sequence of SEQ ID NO: 917 or 918, optionally wherein one of the first immunoglobulin antigen-binding domain and the second immunoglobulin antigen-binding domain comprises a VH region comprising the amino acid sequence of SEQ ID NO: 917; and a VL region comprising the amino acid sequence of SEQ ID NO: 918, and optionally wherein one of the first immunoglobulin antigen-binding domain and the second immunoglobulin antigen-binding domain is an antigen-binding domain of nivolumab; and/or
wherein one of the first immunoglobulin antigen-binding domain and the second immunoglobulin antigen-binding domain is configured to bind to a PD-L1 extracellular domain sequence, optionally wherein one of the first immunoglobulin antigen-binding domain and the second immunoglobulin antigen-binding domain comprises a VH region comprising HVR-1, HVR-2, and HVR-3 of a VH region comprising the amino acid sequence of SEQ ID NO: 921, and a VL region comprising HVR-1, HVR-2, and HVR-3 of a VL region comprising the amino acid sequence of SEQ ID NO: 922, optionally wherein one of the first immunoglobulin antigen-binding domain and the second immunoglobulin antigen-binding domain comprises a sequence that has at least 80, 85, 90, 95, 97, 98, or 99 percent identity to the sequence of SEQ ID NO: 921 or 922, optionally wherein one of the first immunoglobulin antigen-binding domain and the second immunoglobulin antigen-binding domain comprises a VH region comprising the amino acid sequence of SEQ ID NO: 921; and a VL region comprising the amino acid sequence of SEQ ID NO: 922, and optionally wherein one of the first immunoglobulin antigen-binding domain and the second immunoglobulin antigen-binding domain is an antigen-binding domain of atezolizumab; and/or
wherein one of the first immunoglobulin antigen-binding domain and the second immunoglobulin antigen-binding domain is configured to bind to a CD3 extracellular domain sequence, optionally wherein one of the first immunoglobulin antigen-binding domain and the second immunoglobulin antigen-binding domain comprises a VH region comprising HVR-1, HVR-2, and HVR-3 of a VH region comprising the amino acid sequence of any one of SEQ ID NOs: 925, 929, 933, and 937, and a VL region comprising HVR-1, HVR-2, and HVR-3 of a VL region comprising the amino acid sequence of any one of SEQ ID NOs: 926, 930, 934, and 938, optionally wherein one of the first immunoglobulin antigen-binding domain and the second immunoglobulin antigen-binding domain comprises a sequence that has at least 80, 85, 90, 95, 97, 98, or 99 percent identity to the sequence of any one of SEQ ID NOs: 925, 926, 929, 930, 933, 934, 937, and 938, optionally wherein one of the first immunoglobulin antigen-binding domain and the second immunoglobulin antigen-binding domain comprises a VH region comprising the amino acid sequence of any one of SEQ ID NOs: 925, 929, 933, and 937; and a VL region comprising the amino acid sequence of any one of SEQ ID NOs: 926, 930, 934, and 938, and optionally wherein one of the first immunoglobulin antigen-binding domain and the second immunoglobulin antigen-binding domain is an antigen-binding domain of teplizumab, muromonab, otelixizumab, or visilizumab.

49-79. (canceled)

80. The linker polypeptide of claim 9, wherein the first active domain comprises a receptor-binding domain; optionally wherein the receptor-binding domain comprises a cytokine polypeptide sequence; and/or
wherein the receptor-binding domain comprises a modification to prevent disulfide bond formation, and optionally otherwise comprises wild-type sequence; and/or
wherein the receptor-binding domain has at least 80, 85, 90, 95, 97, 98, or 99 percent identity to the sequence of a wild-type receptor-binding domain or to a receptor-binding domain in Table 1, optionally wherein the receptor-binding domain is a wild-type receptor-binding domain; and/or
wherein the receptor-binding domain is a monomeric cytokine, or wherein the receptor-binding domain is a dimeric receptor-binding domain comprising monomers that are associated covalently (optionally via a polypeptide linker) or noncovalently; and/or
wherein the linker polypeptide further comprises an inhibitory polypeptide sequence capable of blocking an activity of the receptor-binding domain, and a second linker between the receptor-binding domain and the inhibitory polypeptide sequence, the second linker comprising a protease-cleavable polypeptide sequence; and/or
wherein the inhibitory polypeptide sequence comprises a cytokine-binding domain, optionally wherein the cytokine-binding domain is a cytokine-binding domain of a cytokine receptor or a cytokine-binding domain of a fibronectin, optionally wherein the cytokine-binding domain is an immunoglobulin cytokine-binding domain, optionally wherein the immunoglobulin cytokine-binding domain comprises a VL region and a VH region that bind the cytokine, and optionally wherein the immunoglobulin cytokine-binding domain is an Fv, scFv, Fab, or VHH.

81-87. (canceled)

88. The linker polypeptide of claim 9, wherein the inhibitory polypeptide sequence comprises a cytokine-binding domain, optionally wherein the cytokine-binding domain is a cytokine-binding domain of a cytokine receptor or a cytokine-binding domain of a fibronectin, optionally wherein the cytokine-binding domain is an immunoglobulin cytokine-binding domain, optionally wherein the immunoglobulin cytokine-binding domain comprises a VL region and a VH region that bind the cytokine, and optionally wherein the immunoglobulin cytokine-binding domain is an Fv, scFv, Fab, or VHH.

89-92. (canceled)

93. The linker polypeptide of claim 80, wherein the linker polypeptide comprises a targeting sequence, wherein the targeting sequence is between the receptor-binding domain and the protease-cleavable polypeptide sequence or one of the protease-cleavable polypeptide sequences; and/or
wherein the receptor-binding domain is an interleukin polypeptide sequence; and/or
wherein the receptor-binding domain is capable of binding a receptor comprising CD132; and/or
wherein the receptor-binding domain is capable of binding a receptor comprising CD122; and/or
wherein the receptor-binding domain is capable of binding a receptor comprising CD25; and/or
wherein the receptor-binding domain is capable of binding a receptor comprising IL-10R; and/or
wherein the receptor-binding domain is capable of binding a receptor comprising IL-15R; and/or
wherein the receptor-binding domain is capable of binding a receptor comprising CXCR3.

94-100. (canceled)

101. The linker polypeptide of claim 80, wherein the receptor-binding domain is an IL-2 polypeptide sequence, optionally wherein the IL-2 polypeptide sequence has at least 80, 85, 90, 95, 97, 98, or 99 percent identity to the sequence of any one of SEQ ID NOs: 2, 1, 3, and 4, optionally wherein the IL-2 polypeptide sequence comprises the sequence of any one of SEQ ID NOs: 2, 1, 3, and 4, optionally wherein the IL-2 polypeptide sequence is a human IL-2 polypeptide sequence, and optionally wherein the IL-2 polypeptide sequence comprises the sequence of SEQ ID NO: 2 or 1; and/or
wherein the inhibitory polypeptide sequence comprises an IL-2 binding domain of an IL-2 receptor (IL-2R), optionally wherein the inhibitory polypeptide sequence comprises an amino acid sequence having at least 80, 85, 90, 95, 97, 98, or 99 percent identity to the sequence of any one of SEQ ID NOs: 10-29 and 40-51, optionally wherein the TL-2R is a human IL-2R; and/or
wherein the inhibitory polypeptide sequence comprises an IL-2-binding immunoglobulin domain, optionally wherein the IL-2-binding immunoglobulin domain is a human IL-2-binding immunoglobulin domain, optionally wherein the IL-2-binding immunoglobulin domain comprises a VH region comprising hypervariable regions (HVRs) HVR-1, HVR-2, and HVR-3 having the sequences of SEQ ID NOs: 37, 38, and 39, respectively, and a VL region comprising HVR-1, HVR-2, and HVR-3 having the sequences of SEQ ID NOs: 34, 35, and 36, respectively, and/or the IL-2-binding immunoglobulin domain comprises a VH region comprising an amino acid sequence having at least 80, 85, 90, 95, 97, 98, or 99 percent identity to the sequence of SEQ ID NO: 33 and a VL region comprising an amino acid sequence having at least 80, 85, 90, 95, 97, 98, or 99 percent identity to the sequence of SEQ ID NO: 32, or a VH region comprising an amino acid sequence having at least 80, 85, 90, 95, 97, 98, or 99 percent identity to the sequence of SEQ ID NO: 749 and a VL region comprising an amino acid sequence having at least 80, 85, 90, 95, 97, 98, or 99 percent identity to the sequence of SEQ ID NO: 748, or the IL-2-binding immunoglobulin domain comprises a VH region comprising the sequence of SEQ ID NO: 33 and a VL region comprising the sequence of SEQ ID NO: 32, or a VH region comprising the sequence of SEQ ID NO: 749 and a VL region comprising the sequence of SEQ ID NO: 748, and optionally wherein the IL-2-binding immunoglobulin domain is an scFv; and/or
wherein the inhibitory polypeptide sequence comprises an IL-2-binding immunoglobulin domain, optionally wherein the IL-2-binding immunoglobulin domain is a human IL-2-binding immunoglobulin domain, optionally wherein the IL-2-binding immunoglobulin domain is an scFv, optionally wherein the IL-2-binding immunoglobulin domain comprises the CDRs of an amino acid sequence of SEQ ID NO: 30, 31, 747, 850-856, or 863-870, optionally wherein the IL-2-binding immunoglobulin domain comprises an amino acid sequence having at least 80, 85, 90, 95, 97, 98, or 99 percent identity to the sequence of SEQ ID NO: 30, 31, 747, 850-856, or 863-870, and optionally wherein the IL-2-binding immunoglobulin domain comprises the sequence of SEQ ID NO: 30, 31, 747, 850-856, or 863-870.

102-118. (canceled)

119. The linker polypeptide of claim 80, wherein the receptor-binding domain is an IL-10 polypeptide sequence, optionally wherein the IL-10 polypeptide sequence has at least 80, 85, 90, 95, 97, 98, or 99 percent identity to the sequence of SEQ ID NO: 900, and optionally wherein the IL-10 polypeptide sequence comprises the sequence of SEQ ID NO: 900; and/or
wherein the IL-10 polypeptide sequence is a human IL-10 polypeptide sequence; and/or
wherein the inhibitory polypeptide sequence comprises an IL-10 binding domain of an IL-10 receptor (IL-10R), optionally wherein the inhibitory polypeptide sequence comprises an amino acid sequence having at least 80, 85, 90, 95, 97, 98, or 99 percent identity to the sequence of SEQ ID NO: 1011 or 1012, and/or the IL-10R is a human IL-10R; and/or
wherein the inhibitory polypeptide sequence comprises an IL-10-binding immunoglobulin domain, optionally wherein the IL-10-binding immunoglobulin domain is a human IL-10-binding immunoglobulin domain, optionally wherein the IL-10-binding immunoglobulin domain comprises a VH region comprising hypervariable regions (HVRs) HVR-1, HVR-2, and HVR-3 having the sequences of SEQ ID NOs: 946, 947, and 948, respectively, and a VL region comprising HVR-1, HVR-2, and HVR-3 having the sequences of SEQ ID NOs: 942, 943, and 944, respectively: optionally wherein the IL-10-binding immunoglobulin domain comprises a VH region comprising an amino acid sequence having at least 80, 85, 90, 95, 97, 98, or 99 percent identity to the sequence of SEQ ID NO: 945 and a VL region comprising an amino acid sequence having at least 80, 85, 90, 95, 97, 98, or 99 percent identity to the sequence of SEQ ID NO: 941, optionally wherein the IL-10-binding immunoglobulin domain comprises a VH region comprising the sequence of SEQ ID NO: 945 and a VL region comprising the sequence of SEQ ID NO: 941, optionally wherein the IL-10-binding immunoglobulin domain is an scFv, optionally wherein the IL-10-binding immunoglobulin domain comprises an amino acid sequence having at least 80, 85, 90, 95, 97, 98, or 99 percent identity to the sequence of SEQ ID NO: 939 or 940, and optionally wherein the IL-10-binding immunoglobulin domain comprises the sequence of SEQ ID NO: 939 or 940.

120-133. (canceled)

134. The linker polypeptide of claim 80, wherein the receptor-binding domain is an IL-15 polypeptide sequence, optionally wherein the IL-15 polypeptide sequence is a human IL-15 polypeptide sequence, optionally wherein the IL-15 polypeptide sequence has at least 80, 85, 90, 95, 97, 98, or 99 percent identity to the sequence of SEQ ID NO: 901, and optionally wherein the IL-15 polypeptide sequence comprises the sequence of SEQ ID NO: 901; and/or
wherein the inhibitory polypeptide sequence comprises an IL-15 binding domain of an IL-15 receptor (IL-15R), optionally wherein the IL-15R is a human IL-15R, and optionally wherein the inhibitory polypeptide sequence comprises an amino acid sequence having at least 80, 85, 90, 95, 97, 98, or 99 percent identity to the sequence of any one of SEQ ID NOs: 1016-1019; and/or
wherein the inhibitory polypeptide sequence comprises an IL-15-binding immunoglobulin domain, optionally wherein the IL-15-binding immunoglobulin domain is a human IL-15-binding immunoglobulin domain, optionally wherein the IL-15-binding immunoglobulin domain comprises a VH region comprising HVR-1, HVR-2, and HVR-3 of a VH region comprising the amino acid sequence of any one of SEQ ID NOs: 950, 955, 957, 960, 963, 966, 969, 972, 975, 978, 981, 985, and 988, and a VL region comprising HVR-1, HVR-2, and HVR-3 of a VL region comprising the amino acid sequence of any one of SEQ ID NOs: 952, 954, 958, 961, 964, 967, 970, 973, 976, 979, 982, 984, and 987, optionally wherein the IL-15-binding immunoglobulin domain comprises a VH region comprising an amino acid sequence having at least 80, 85, 90, 95, 97, 98, or 99 percent identity to the sequence of any one of SEQ ID NOs: 950, 955, 957, 960, 963, 966, 969, 972, 975, 978, 981, 985, and 988 and a VL region comprising an amino acid sequence having at least 80, 85, 90, 95, 97, 98, or 99 percent identity to the sequence of any one of SEQ ID NOs: 952, 954, 958, 961, 964, 967, 970, 973, 976, 979, 982, 984, and 987, optionally wherein the IL-15-binding immunoglobulin domain comprises a VH region comprising the sequence of any one of SEQ ID NOs: 950, 955, 957, 960, 963, 966, 969, 972, 975, 978, 981, 985, and 988 and a VL region comprising the sequence of any one of SEQ ID NOs: 952, 954, 958, 961, 964, 967, 970, 973, 976, 979, 982, 984, and 987, optionally wherein the IL-15-binding immunoglobulin domain is an scFv, optionally wherein the IL-15-binding immunoglobulin domain comprises an amino acid sequence having at least 80, 85, 90, 95, 97, 98, or 99 percent identity to the sequence of any one of SEQ ID NOs: 953, 956, 959, 962, 965, 968, 971, 974, 977, 980, 983, and 986, and optionally wherein the IL-15-binding immunoglobulin domain comprises the sequence of any one of SEQ ID NOs: 953, 956, 959, 962, 965, 968, 971, 974, 977, 980, 983, and 986.

135-148. (canceled)

149. The linker polypeptide of claim 80, wherein the receptor-binding domain is a CXCL9 polypeptide sequence, optionally wherein the CXCL9 polypeptide sequence is a human CXCL9 polypeptide sequence, optionally wherein the CXCL9 polypeptide sequence has at least 80, 85, 90, 95, 97, 98, or 99 percent identity to the sequence of SEQ ID NO: 902, and optionally wherein the CXCL9 polypeptide sequence comprises the sequence of SEQ ID NO: 902; and/or
wherein the inhibitory polypeptide sequence comprises a CXCL9 binding domain of CXCR3, optionally wherein the CXCR3 is a human CXCR3, optionally wherein the inhibitory polypeptide sequence comprises an amino acid sequence having at least 80, 85, 90, 95, 97, 98, or 99 percent identity to the sequence of SEQ ID NO: 1020 or 1021; and/or
wherein the inhibitory polypeptide sequence comprises an CXCL9-binding immunoglobulin domain, optionally wherein the CXCL9-binding immunoglobulin domain is a human CXCL9-binding immunoglobulin domain.

150-157. (canceled)

158. The linker polypeptide of claim 80, wherein the receptor-binding domain is a CXCL10 polypeptide sequence, optionally wherein the CXCL10 polypeptide sequence is a human CXCL10 polypeptide sequence, optionally wherein the CXCL10 polypeptide sequence has at least 80, 85, 90, 95, 97, 98, or 99 percent identity to the sequence of SEQ ID NO: 903, and optionally wherein the CXCL10 polypeptide sequence comprises the sequence of SEQ ID NO: 903; and/or
wherein the inhibitory polypeptide sequence comprises an CXCL10 binding domain of CXCR3, optionally wherein the CXCR3 is a human CXCR3, and optionally wherein the inhibitory polypeptide sequence comprises an amino acid sequence having at least 80, 85, 90, 95, 97, 98, or 99 percent identity to the sequence of SEQ ID NO: 1020 or 1021; and/or
wherein the inhibitory polypeptide sequence comprises an CXCL10-binding immunoglobulin domain, optionally wherein the CXCL10-binding immunoglobulin domain is a human CXCL10-binding immunoglobulin domain, optionally wherein the CXCL10-binding immunoglobulin domain comprises a VH region comprising hypervariable regions (HVRs) HVR-1, HVR-2, and HVR-3 having the sequences of SEQ ID NOs: 993, 994, and 995, respectively, and a VL region comprising HVR-1, HVR-2, and HVR-3 having the sequences of SEQ ID NOs: 996, 997, and 998, respectively, optionally wherein the CXCL10-binding immunoglobulin domain comprises a VH region comprising an amino acid sequence having at least 80, 85, 90, 95, 97, 98, or 99 percent identity to the sequence of SEQ ID NO: 991 and a VL region comprising an amino acid sequence having at least 80, 85, 90, 95, 97, 98, or 99 percent identity to the sequence of SEQ ID NO: 992, optionally wherein the CXCL10-binding immunoglobulin domain comprises a VH region comprising the sequence of SEQ ID NO: 991 and a VL region comprising the sequence of SEQ ID NO: 992, optionally wherein the CXCL10-binding immunoglobulin domain is an scFv, optionally wherein the CXCL10-binding immunoglobulin domain comprises an amino acid sequence having at least 80, 85, 90, 95, 97, 98, or 99 percent identity to the sequence of SEQ ID NO: 989 or 990, and optionally wherein the CXCL10-binding immunoglobulin domain comprises the sequence of SEQ ID NO: 989 or 990.

159-172. (canceled)

173. The linker polypeptide of claim 9, wherein the inhibitory polypeptide sequence interferes with binding between the first active domain and a receptor of the first active domain and/or with binding between the second active domain and a receptor of the second active domain; and/or
wherein the inhibitory polypeptide sequence and the pharmacokinetic modulator are different elements of the linker polypeptide; and/or
wherein the inhibitory polypeptide sequence comprises a steric blocker; and/or
wherein the inhibitory polypeptide sequence comprises at least a portion of the pharmacokinetic modulator.

174-176. (canceled)

177. The linker polypeptide of claim 9, wherein the pharmacokinetic modulator comprises at least a portion of an immunoglobulin constant domain, optionally wherein the pharmacokinetic modulator comprises at least a portion of an immunoglobulin Fc region, and optionally wherein the pharmacokinetic modulator comprises an immunoglobulin Fc region; and/or
wherein the immunoglobulin is a human immunoglobulin; and/or
wherein the immunoglobulin is IgG, optionally wherein the IgG is IgG1, IgG2, IgG3, or IgG4.

178-182. (canceled)

183. The linker polypeptide of claim 9, wherein the linker polypeptide further comprises a growth factor-binding polypeptide sequence or a growth factor receptor-binding polypeptide sequence; optionally
wherein the growth factor-binding polypeptide sequence comprises a TGF-βR extracellular domain sequence, and optionally wherein the TGF-βR extracellular domain sequence comprises an amino acid sequence having at least 80, 85, 90, 95, 97, 98, or 99 percent identity to the sequence of SEQ ID NO: 1022 or 1023; and/or
wherein the growth factor-binding polypeptide sequence comprises a growth factor-binding immunoglobulin domain, optionally wherein the growth factor-binding immunoglobulin domain is configured to bind to a TGF-β, optionally wherein the growth factor-binding immunoglobulin domain comprises a VH region comprising HVR-1, HVR-2, and HVR-3 of a VH region comprising the amino acid sequence of SEQ ID NO: 1008, and a VL region comprising HVR-1, HVR-2, and HVR-3 of a VL region comprising the amino acid sequence of SEQ ID NO: 1010, optionally wherein the growth factor-binding immunoglobulin domain comprises a VH region comprising the amino acid sequence of SEQ ID NO: 1008; and a VL region comprising the amino acid sequence of SEQ ID NO: 1010, and optionally wherein the growth factor-binding immunoglobulin domain comprises a sequence that has at least 80, 85, 90, 95, 97, 98, or 99 percent identity to the sequence of SEQ ID NO: 1007 or 1009; and/or
wherein the growth factor receptor-binding polypeptide sequence comprises a TGF-β sequence, optionally wherein the TGF-β sequence comprises an amino acid sequence having at least 80, 85, 90, 95, 97, 98, or 99 percent identity to the sequence of any one of SEQ ID NOs. 904-906; and/or
wherein the growth factor receptor-binding polypeptide sequence comprises a growth factor receptor-binding immunoglobulin domain, optionally wherein the growth factor receptor-binding immunoglobulin domain is configured to bind to a TGF-βR extracellular domain sequence, optionally wherein the growth factor receptor-binding immunoglobulin domain comprises a VH region comprising HVR-1, HVR-2, and HVR-3 of a VH region comprising the amino acid sequence of SEQ ID NO: 999 or 1003, and a VL region comprising HVR-1, HVR-2, and HVR-3 of a VL region comprising the amino acid sequence of SEQ ID NO: 1000 or 1004, optionally wherein the growth factor receptor-binding immunoglobulin domain comprises a VH region comprising the amino acid sequence of SEQ ID NO: 999 or 1003; and a VL region comprising the amino acid sequence of SEQ ID NO: 1000 or 1004, and optionally wherein the growth factor receptor-binding immunoglobulin domain comprises a sequence that has at least 80, 85, 90, 95, 97, 98, or 99 percent identity to the sequence of any one of SEQ ID NOs: 1001, 1002, 1005, and 1006.

184-197. (canceled)

198. The linker polypeptide of claim 9, comprising a plurality of protease-cleavable polypeptide sequences; and/or
wherein the protease-cleavable polypeptide sequence is C-terminal to a VH region, C-terminal to at least a portion of a CH1 domain, between a CH1 domain and a CH2 domain, N-terminal to at least a portion of a CH2 domain, N-terminal to a disulfide bond between heavy chains, N-terminal to a disulfide bond within a CH2 domain, or N-terminal to a hinge region, or is within a hinge region; and/or
wherein the protease-cleavable polypeptide sequence is C-terminal to the first targeting sequence and to the second targeting sequence; and/or
wherein the protease-cleavable polypeptide sequence is N-terminal to the first targeting sequence and to the second targeting sequence; and/or
wherein the protease-cleavable polypeptide sequence is C-terminal to a first plurality of targeting sequences and is N-terminal to a second plurality of targeting sequences; and/or
wherein the protease-cleavable polypeptide sequence is C-terminal to a plurality of targeting sequences and is N-terminal to at least one targeting sequence; and/or
wherein the protease-cleavable polypeptide sequence is N-terminal to a plurality of targeting sequences and is C-terminal to at least one targeting sequence; and/or
wherein the protease-cleavable polypeptide sequence is C-terminal to the first targeting sequence and to the second targeting sequence and is not N-terminal to a targeting sequence; and/or
wherein the protease-cleavable polypeptide sequence is N-terminal to the first targeting sequence and to the second targeting sequence and is not C-terminal to a targeting sequence.

199-206. (canceled)

207. The linker polypeptide of claim 9, wherein the linker polypeptide is configured to release the first active domain from a remaining portion of the linker polypeptide upon cleavage of the protease-cleavable polypeptide sequence, optionally wherein the first active domain is configured to remain connected to one or more of: one of the first targeting sequence and the second targeting sequence, one of the at least one targeting sequence, one of the first plurality of targeting sequences, one of the second plurality of targeting sequences, one of the plurality of targeting sequences, and the pharmacokinetic modulator upon cleavage of the protease-cleavable polypeptide sequence; and/or
wherein the linker polypeptide is configured to release the second active domain from a remaining portion of the linker polypeptide upon cleavage of the protease-cleavable polypeptide sequence, optionally wherein the second active domain is configured to remain connected to one or more of: one of the first targeting sequence and the second targeting sequence, one of the at least one targeting sequence, one of the first plurality of targeting sequences, one of the second plurality of targeting sequences, one of the plurality of targeting sequences, and the pharmacokinetic modulator upon cleavage of the protease-cleavable polypeptide sequence.

208-210. (canceled)

211. The linker polypeptide of claim 9, wherein the protease-cleavable polypeptide sequence is recognized by a metalloprotease, a serine protease, a cysteine protease, an aspartate protease, a threonine protease, a glutamate protease, a gelatinase, an asparagine peptide lyase, a cathepsin, a kallikrein, a plasmin, a collagenase, a hK1, a hK10, a hK15, a stromelysin, a Factor Xa, a chymotrypsin-like protease, a trypsin-like protease, a elastase-like protease, a subtilisin-like protease, an actinidain, a bromelain, a calpain, a caspase, a Mir 1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, an ADAM 10, an ADAM 17, an ADAM 12, an urokinase plasminogen activator (uPA), an enterokinase, a prostate-specific target (PSA, hK3), an interleukin-1b converting enzyme, a thrombin, a FAP (FAP-a), a dipeptidyl peptidase, or dipeptidyl peptidase IV (DPPIV/CD26), a type II transmembrane serine protease (TTSP), a neutrophil elastase, a proteinase 3, a mast cell chymase, a mast cell tryptase, or a dipeptidyl peptidase; and/or
wherein the protease-cleavable polypeptide sequence comprises the sequence of any one of SEQ ID NOs: 80-94 or a variant sequence having one or two mismatches relative to the sequence of any one of SEQ ID NOs: 80-90; and/or
wherein the protease-cleavable polypeptide sequence comprises the sequence of any one of SEQ ID NOs: 701-742, or a variant having one or two mismatches relative to the sequence of any one of SEQ ID NOs: 701-742; and/or
wherein the protease-cleavable polypeptide sequence is recognized by a matrix metalloprotease; and/or
wherein the protease-cleavable polypeptide sequence is recognized by MMP-1; and/or
wherein the protease-cleavable polypeptide sequence is recognized by MMP-2; and/or
wherein the protease-cleavable polypeptide sequence is recognized by MMP-3; and/or
wherein the protease-cleavable polypeptide sequence is recognized by MMP-7; and/or
wherein the protease-cleavable polypeptide sequence is recognized by MMP-8; and/or
wherein the protease-cleavable polypeptide sequence is recognized by MMP-9; and/or
wherein the protease-cleavable polypeptide sequence is recognized by MMP-12; and/or
wherein the protease-cleavable polypeptide sequence is recognized by MMP-13; and/or
wherein the protease-cleavable polypeptide sequence is recognized by MMP-14; and/or
wherein the protease-cleavable polypeptide sequence is recognized by more than one MMP; and/or
wherein the protease-cleavable polypeptide sequence is recognized by two, three, four, five, six, or seven of MMP-2, MMP-7, MMP-8, MMP-9, MMP-12, MMP-13, and MMP-14.

212-241. (canceled)

242. The linker polypeptide of claim 9, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind an extracellular matrix component, heparin, an integrin, or a syndecan; or is configured to bind, in a pH-sensitive manner, an extracellular matrix component, heparin, IgB (CD79b), an integrin, a cadherin, a heparan sulfate proteoglycan, a syndecan, or a fibronectin; or the targeting sequence comprises the sequence of any one of SEQ ID NOs: 179-665 or a variant having one or two mismatches relative to the sequence of any one of SEQ ID NOs: 179-665; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently comprises the sequence of any one of SEQ ID NOs: 179-665, or a variant having one or two mismatches relative to the sequence of any one of SEQ ID NOs: 179-665; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently comprises the sequence of any one of SEQ ID NOs: 179-665; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently comprises the sequence of any one of SEQ ID NOs: 200, 330, 619, 653, and 663-665, or a variant having one or two mismatches relative to the sequence of any one of SEQ ID NOs: 200, 330, 619, 653, and 663-665; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently comprises the sequence of any one of SEQ ID NOs: 200, 330, 619, 653, and 663-665; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to denatured collagen or to collagen, optionally wherein the collagen is collagen I, collagen II, collagen III, or collagen IV.

243-252. (canceled)

253. The linker polypeptide of claim 9, wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to integrin, optionally wherein the integrin is one or more of α1β1 integrin, α2β1 integrin, α3β 1 integrin, α4β1 integrin, α5β1 integrin, α6β1 integrin, α7β1 integrin, α9β1 integrin, α4β7 integrin, αvβ3 integrin, αvβ5 integrin, αIIbβ3 integrin, αIIIbβ3 integrin, αMβ2 integrin, or αIIbβ3 integrin; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to von Willebrand factor; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to IgB; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to heparin; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to heparin and a syndecan, a heparan sulfate proteoglycan, or an integrin, optionally wherein the integrin is one or more of α1β1 integrin, α2β1 integrin, α3β1 integrin, α4β1 integrin, α5β1 integrin, α6β1 integrin, α7β1 integrin, α9β1 integrin, α4β7 integrin, αvβ3 integrin, αvβ5 integrin, αIIbβ3 integrin, αIIbβ3 integrin, αMβ2 integrin, or αIIbβ3 integrin, and optionally wherein the syndecan is one of more of syndecan-1, syndecan-4, and syndecan-2(w); and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to a heparan sulfate proteoglycan; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to a sulfated glycoprotein; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to hyaluronic acid; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to fibronectin; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind to cadherin; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target in a pH-sensitive manner; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently has a higher affinity for its target at a pH below normal physiological pH than at normal physiological pH, optionally wherein the pH below normal physiological pH is below 7, or below 6; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently has a higher affinity for its target at a pH in the range of 5-7, e.g., 5-5.5, 5.5-6, 6-6.5, or 6.5-7, than at normal physiological pH; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently comprises one or more histidines, e.g., 1, 2, 3, 4, 5, 6, 7, 8, 9, or 10 histidines; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently comprises the sequence of any one of SEQ ID NOs: 641-663, or a variant having one or two mismatches relative to the sequence of any one of SEQ ID NOs: 641-663; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently comprises the sequence of any one of SEQ ID NOs: 641-665; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind, in a pH-sensitive manner, an extracellular matrix component, IgB (CD79b), an integrin, a cadherin, a heparan sulfate proteoglycan, a syndecan, or a fibronectin, optionally wherein the extracellular matrix component is hyaluronic acid, heparin, heparan sulfate, or a sulfated glycoprotein; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind a fibronectin in a pH-sensitive manner; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently is configured to bind its target with an affinity from 0.1 nM to 1 nM, from 1 nM to 10 nM, from 10 nM to 100 nM, from 100 nM to 1 μM, from 1 μM to 10 μM, or from 10 μM to 100 μM.

254-281. (canceled)

282. The linker polypeptide of claim 9, wherein at least one of the first linker and the second linker comprises one of the first targeting sequence and the second targeting sequence, one of the at least one targeting sequence, one of the first plurality of targeting sequences, one of the second plurality of targeting sequences, or one of the plurality of targeting sequences; and/or
wherein the protease-cleavable polypeptide sequence comprises one of the first targeting sequence and the second targeting sequence, one of the at least one targeting sequence, one of the first plurality of targeting sequences, one of the second plurality of targeting sequences, or one of the plurality of targeting sequences; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences increases a serum half-life of the linker polypeptide; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences synergistically increases a serum half-life of the linker polypeptide together with the pharmacokinetic modulator or with another one of the first targeting sequence and the second targeting sequence, another one of the at least one targeting sequence, another one of the first plurality of targeting sequences, another one of the second plurality of targeting sequences, or another one of the plurality of targeting sequences; and/or
wherein one or each of the first targeting sequence and the second targeting sequence, one or each of the at least one targeting sequence, one or each of the first plurality of targeting sequences, one or each of the second plurality of targeting sequences, or one or each of the plurality of targeting sequences independently increases a serum half-life of the linker polypeptide.

283-286. (canceled)

287. The linker polypeptide of claim 9, further comprising a blocker conjugated to one of or each of the first active domain and the second active domain,
optionally wherein the blocker is conjugated to one of or each of the first active domain and the second active domain via a protease-cleavable polypeptide sequence, and
optionally wherein the blocker is an albumin, a serum albumin, and/or a human albumin.

288-291. (canceled)

292. The linker polypeptide of claim 9, further comprising a chemotherapy drug, optionally wherein the chemotherapy drug is conjugated to the pharmacokinetic modulator, and optionally wherein the chemotherapy drug is selected from altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, epirubicin, idarubicin, valrubicin, bleomycin, dactinomycin, mitomycin-c, mitoxantrone, irinotecan, topotecan, etoposide, mitoxantrone, teniposide, cabazitaxel, docetaxel, paclitaxel, vinblastine, vincristine, vinorelbine, prednisone, methylprednisolone, dexamethasone, retinoic acid, arsenic trioxide, asparaginase, eribulin, hydroxyurea, ixabepilone, mitotane, omacetaxine, pegaspargase, procarbazine, romidepsin, and vorinostat.

293-294. (canceled)

295. The linker polypeptide of claim 9, wherein a molecular weight of one or each of the first active domain and the second active domain independently is about or less than 14 kDa, about 12 kDa to about 14 kDa, about 10 kDa to about 12 kDa, about 8 kDa to about 10 kDa, about 6 kDa to about 8 kDa, about 4 kDa to about 6 kDa, about 2 kDa to about 4 kDa, or about 800 Da to about 2 kDa: or
wherein a molecular weight of one or each of the first active domain and the second active domain independently is about or greater than 16 kDa, about 16 kDa to about 18 kDa, about 18 kDa to about 20 kDa, about 20 kDa to about 22 kDa, about 22 kDa to about 24 kDa, about 24 kDa to about 26 kDa, about 26 kDa to about 28 kDa, about 28 kDa to about 30 kDa, about 30 kDa to about 50 kDa, about 50 kDa to about 100 kDa, about 100 kDa to about 150 kDa, about 150 kDa to about 200 kDa, about 200 kDa to about 250 kDa, or about 250 kDa to about 300 kDa.

296-316. (canceled)

317. The linker polypeptide of claim 9, comprising a combined targeting sequence and protease cleavable sequence, wherein the combined targeting sequence and protease cleavable sequence is any one of SEQ ID NOs: 667-673.

318. A linker polypeptide comprising an amino acid sequence having at least 80, 85, 90, 95, 97, 98, or 99 percent identity to the sequence of any one of SEQ ID NOs: 800-848 and 1024-1137, optionally wherein the linker polypeptide comprises the sequence of any one of SEQ ID NOs: 800-848 and 1024-1137, optionally wherein the linker polypeptide comprises the sequence of SEQ ID NO: 1119.

319. (canceled)

320. A pharmaceutical composition comprising the linker polypeptide of claim 9.

321-322. (canceled)

323. A method of treating a cancer, comprising administering the linker polypeptide of claim 9 to a subject in need thereof.

324. (canceled)

325. The method of claim 323, wherein the cancer is a solid tumor, optionally wherein the solid tumor is metastatic and/or unresectable; and/or
wherein the cancer is a PD-L1-expressing cancer; and/or
wherein the cancer is a melanoma, a colorectal cancer, a breast cancer, a pancreatic cancer, a lung cancer, a prostate cancer, an ovarian cancer, a cervical cancer, a gastric or gastrointestinal cancer, a lymphoma, a colon or colorectal cancer, an endometrial cancer, a thyroid cancer, or a bladder cancer; and/or
wherein the cancer is a microsatellite instability-high cancer; and/or
wherein the cancer is mismatch repair deficient.

326-330. (canceled)

331. A nucleic acid encoding the linker polypeptide of claim 9, or an expression vector comprising a nucleic acid encoding the linker polypeptide of claim 9.

332. (canceled)

333. A host cell comprising the nucleic acid or the vector of claim 331.

334. A method of producing a linker polypeptide, comprising culturing the host cell of claim 333 under conditions wherein the linker polypeptide is produced, optionally further comprising isolating the linker polypeptide.

335. (canceled)
